SEQUENCE LISTING (172 sequences)

SEQ ID NO: 1 - DNA, 16 bp, Artificial Sequence
  Synthetic
caacgcacccaacaca

SEQ ID NO: 2 - DNA, 15 bp, Artificial Sequence
  Synthetic
gcggagtttggggga

SEQ ID NO: 3 - DNA, 15 bp, Artificial Sequence
  Synthetic
tacgtggcgggttgg

SEQ ID NO: 4 - DNA, 23 bp, Artificial Sequence
  Synthetic
aaaaaaacaaccttaatatcttc

SEQ ID NO: 5 - DNA, 20 bp, Artificial Sequence
  Synthetic
gtttttttggttcggagttg

SEQ ID NO: 6 - DNA, 23 bp, Artificial Sequence
  Synthetic
caaaacgaaacacgaaaaaaata

SEQ ID NO: 7 - DNA, 20 bp, Artificial Sequence
  Synthetic
gtagttggatgggattattt

SEQ ID NO: 8 - DNA, 17 bp, Artificial Sequence
  Synthetic
cacccgcaaaatcctct

SEQ ID NO: 9 - DNA, 20 bp, Artificial Sequence
  Synthetic
ggagtgtagaaattaataag

SEQ ID NO: 10 - DNA, 18 bp, Artificial Sequence
  Synthetic
ctcgcttcctcctcctac

SEQ ID NO: 11 - DNA, 15 bp, Artificial Sequence
  Synthetic
cggagtcgagtttag

SEQ ID NO: 12 - DNA, 14 bp, Artificial Sequence
  Synthetic
accgccgacccttt

SEQ ID NO: 13 - DNA, 19 bp, Artificial Sequence
  Synthetic
gtaatatttagggattggg

SEQ ID NO: 14 - DNA, 20 bp, Artificial Sequence
  Synthetic
ctccttataacaacaacttc

SEQ ID NO: 15 - DNA, 17 bp, Artificial Sequence
  Synthetic
gagggtgggtttggtaa

SEQ ID NO: 16 - DNA, 20 bp, Artificial Sequence
  Synthetic
gatataaaaacaaccctcca

SEQ ID NO: 17 - DNA, 20 bp, Artificial Sequence
  Synthetic
gggaagaaagggggttttgt

SEQ ID NO: 18 - DNA, 20 bp, Artificial Sequence
  Synthetic
tacgacgaaaactacgcgaa

SEQ ID NO: 19 - DNA, 20 bp, Artificial Sequence
  Synthetic
aacatccgttcaaactaaca

SEQ ID NO: 20 - DNA, 17 bp, Artificial Sequence
  Synthetic
ggttgtgcgaagttgag

SEQ ID NO: 21 - DNA, 24 bp, Artificial Sequence
  Synthetic
gttttaggagttatttgggtttgc

SEQ ID NO: 22 - DNA, 25 bp, Artificial Sequence
  Synthetic
actataacacctcgctactaacgct

SEQ ID NO: 23 - DNA, 23 bp, Artificial Sequence
  Synthetic
tttttgaaagtttgagaaaatgt

SEQ ID NO: 24 - DNA, 16 bp, Artificial Sequence
  Synthetic
ccgacgcctctaccaa

SEQ ID NO: 25 - DNA, 21 bp, Artificial Sequence
  Synthetic
ttgttggagygttaggtttgg

SEQ ID NO: 26 - DNA, 22 bp, Artificial Sequence
  Synthetic
ccraaaaaaccttaaactcccc

SEQ ID NO: 27 - DNA, 20 bp, Artificial Sequence
  Synthetic
ttatttcggggaaggttacg

SEQ ID NO: 28 - DNA, 25 bp, Artificial Sequence
  Synthetic
gcgaaaacgaaatcataaaataaac

SEQ ID NO: 29 - DNA, 16 bp, Artificial Sequence
  Synthetic
cgagtcgagtttgggt

SEQ ID NO: 30 - DNA, 19 bp, Artificial Sequence
  Synthetic
acctccgaaacaaaatcta

SEQ ID NO: 31 - DNA, 20 bp, Artificial Sequence
  Synthetic
tgttagagtttattgggatg

SEQ ID NO: 32 - DNA, 20 bp, Artificial Sequence
  Synthetic
gaaaaccgaatctcaaacac

SEQ ID NO: 33 - DNA, 22 bp, Artificial Sequence
  Synthetic
atacgggagaaagagtacgtta

SEQ ID NO: 34 - DNA, 23 bp, Artificial Sequence
  Synthetic
aacgtaaccgtacaacctaaacg

SEQ ID NO: 35 - DNA, 20 bp, Artificial Sequence
  Synthetic
tagttttcggagaagacggc

SEQ ID NO: 36 - DNA, 20 bp, Artificial Sequence
  Synthetic
ctataaccctacgatcgcct

SEQ ID NO: 37 - DNA, 23 bp, Artificial Sequence
  Synthetic
agggagtttaatagcgatcgagt

SEQ ID NO: 38 - DNA, 21 bp, Artificial Sequence
  Synthetic
tttactaaacaccccgaaaac

SEQ ID NO: 39 - DNA, 20 bp, Artificial Sequence
  Synthetic
aggtcggtttttatatggtg

SEQ ID NO: 40 - DNA, 21 bp, Artificial Sequence
  Synthetic
tcgatataactactccaaatc

SEQ ID NO: 41 - DNA, 16 bp, Artificial Sequence
  Synthetic
gggagggggtagtagg

SEQ ID NO: 42 - DNA, 25 bp, Artificial Sequence
  Synthetic
cgctcatttaatttaaatttatttc

SEQ ID NO: 43 - DNA, 17 bp, Artificial Sequence
  Synthetic
gggcgcgatagtttgag

SEQ ID NO: 44 - DNA, 14 bp, Artificial Sequence
  Synthetic
cccgcgccctttcc

SEQ ID NO: 45 - DNA, 17 bp, Artificial Sequence
  Synthetic
ggggcgcggttttttta

SEQ ID NO: 46 - DNA, 20 bp, Artificial Sequence
  Synthetic
ctaaactacgctaaattcct

SEQ ID NO: 47 - DNA, 20 bp, Artificial Sequence
  Synthetic
agggatttaggttaggggtc

SEQ ID NO: 48 - DNA, 20 bp, Artificial Sequence
  Synthetic
acgacatccttcaaaccgac

SEQ ID NO: 49 - DNA, 16 bp, Artificial Sequence
  Synthetic
aggttcgggtgaggag

SEQ ID NO: 50 - DNA, 20 bp, Artificial Sequence
  Synthetic
aaacgtctatcccaaaactt

SEQ ID NO: 51 - DNA, 20 bp, Artificial Sequence
  Synthetic
agttaaaagtaagggtagga

SEQ ID NO: 52 - DNA, 19 bp, Artificial Sequence
  Synthetic
ccccgctaaaaattaacca

SEQ ID NO: 53 - DNA, 17 bp, Artificial Sequence
  Synthetic
ggtcgggttgagattgg

SEQ ID NO: 54 - DNA, 17 bp, Artificial Sequence
  Synthetic
ggtggggttgagattgg

SEQ ID NO: 55 - DNA, 24 bp, Artificial Sequence
  Synthetic
gtgatggaggaggtttagtaagtt

SEQ ID NO: 56 - DNA, 25 bp, Artificial Sequence
  Synthetic
ccaataaaacctactcctcccttaa

SEQ ID NO: 57 - DNA, 20 bp, Artificial Sequence
  Synthetic
gtcgattcgcgttttcgtcg

SEQ ID NO: 58 - DNA, 17 bp, Artificial Sequence
  Synthetic
tcggttttttcgcggcg

SEQ ID NO: 59 - DNA, 16 bp, Artificial Sequence
  Synthetic
cgccccgtcgccgaat

SEQ ID NO: 60 - DNA, 18 bp, Artificial Sequence
  Synthetic
ttgttgcggtcgcggacg

SEQ ID NO: 61 - DNA, 19 bp, Artificial Sequence
  Synthetic
agggcgtcgcgttttcggg

SEQ ID NO: 62 - DNA, 18 bp, Artificial Sequence
  Synthetic
tttcgatttcgcgcgggg

SEQ ID NO: 63 - DNA, 15 bp, Artificial Sequence
  Synthetic
tgcgccggagacgcg

SEQ ID NO: 64 - DNA, 22 bp, Artificial Sequence
  Synthetic
cgcgttcgagttaagagtcgcg

SEQ ID NO: 65 - DNA, 15 bp, Artificial Sequence
  Synthetic
cgtcgggagggtcgg

SEQ ID NO: 66 - DNA, 25 bp, Artificial Sequence
  Synthetic
cgtcgtcgttttcggattttgtacg

SEQ ID NO: 67 - DNA, 21 bp, Artificial Sequence
  Synthetic
cggtggttcgtaggggtcgcg

SEQ ID NO: 68 - DNA, 15 bp, Artificial Sequence
  Synthetic
cgtagcgcggcgggg

SEQ ID NO: 69 - DNA, 20 bp, Artificial Sequence
  Synthetic
ttcgttatttgggtcgcggg

SEQ ID NO: 70 - DNA, 20 bp, Artificial Sequence
  Synthetic
cgacgccgaccgcgccctcg

SEQ ID NO: 71 - DNA, 17 bp, Artificial Sequence
  Synthetic
tcggacgcgttttcggg

SEQ ID NO: 72 - DNA, 23 bp, Artificial Sequence
  Synthetic
cgcgtagttatcgttagacggcg

SEQ ID NO: 73 - DNA, 22 bp, Artificial Sequence
  Synthetic
tcgaaaagacgcgtggtttcgt

SEQ ID NO: 74 - DNA, 17 bp, Artificial Sequence
  Synthetic
ggttacgcggcgcgtgg

SEQ ID NO: 75 - DNA, 19 bp, Artificial Sequence
  Synthetic
tcgtggtagcgttacgcga

SEQ ID NO: 76 - DNA, 21 bp, Artificial Sequence
  Synthetic
agacgggcgttttttgtgcga

SEQ ID NO: 77 - DNA, 22 bp, Artificial Sequence
  Synthetic
tcgttaattagtatcgcgacga

SEQ ID NO: 78 - DNA, 19 bp, Artificial Sequence
  Synthetic
agtcggtcgaggttttcgt

SEQ ID NO: 79 - DNA, 18 bp, Artificial Sequence
  Synthetic
tgttttgggcgcgttcga

SEQ ID NO: 80 - DNA, 14 bp, Artificial Sequence
  Synthetic
cgcgttcggggcgt

SEQ ID NO: 81 - DNA, 17 bp, Artificial Sequence
  Synthetic
ttcgtttcggggcgggg

SEQ ID NO: 82 - DNA, 23 bp, Artificial Sequence
  Synthetic
cgttttgtcgttgtaggtttcgt

SEQ ID NO: 83 - DNA, 24 bp, Artificial Sequence
  Synthetic
cgggggttttaaatttacgtttcg

SEQ ID NO: 84 - DNA, 20 bp, Artificial Sequence
  Synthetic
cggtttttgtcggggtgcgg

SEQ ID NO: 85 - DNA, 30 bp, Artificial Sequence
  Synthetic
accaccacccaacacacaataacaaacaca

SEQ ID NO: 86 - DNA, 7316 bp, Homo sapiens
gcactgttctgcttttcactgttaatccaacctgctcccaaacatcacccctcctccgcagagtgctgaagatgaagagagcccagcggcacaggggacccagagcttgcccttctgctgactcagtagtgactccgctgcctgtcatgactgatgtttccagaagggtcagggcctcctcaacctacctcttccttatgcacctttgtttgatgcaatagccctggggactctcacgattacgtttcttcttcatctattgtctccacttttatttatttatttatgtattttttagactctatcacccaggctggagtgcagcagcgcgatctcggctcactgcaacgtccgcctcctgggttcaagtgattctcctgcctcagcctcctgagtagctaggattacaggtatgtgcaccacgcccagttaatttttgtatttttagtagagatggggtttcaccatgttggccaggctggtcttgaactcctgacctcaggtgatccacccacctcggcccccaaatgtgctgggattacaggcatgagccaccgcgcccggccctactgtctccacttttaggagttcaaatccacaacctgactcactggaatgctcacaggagttcatgaactatctgagctacatggatagtctgcatgagttcaaggccactgcagacaccatgtctcagacaggcgtgtcatagtgtcaaagctcctgttaggcagcctgcagaacagcctaggccccgtgaattatgtcactagaacatcaacaattaattttgtccaatgacaaagtctgaaatgactgaaaagggtaggactcaggtaagcctgaaagaatctctcatgcgataataacaatttcatcatgaataataataatgaccatgtattgaacacttaaacttaagtcatgcgtctggcactatattaagcattttctttatattctgttatttaatctttacaggaatcctgaggtcagcatcatcccaatttcacagagtatgacatgacgtctcagagagggtaagcagcttgccaaacattccacagccaggaagctgtaggaccagcctgtgctcctaaggactggctcctggctgctatatatagaactaagcctggtggaggtgtcagaaatagaggttcacttctgtcatcaccgacctccctccacacctttgcaaaggaggaaactgagaggcagggatttccgcagagcaaggaacccaaattgctgcctcctgtgatttatacactgcaccccaagctgtaggggtaacccaaggacaaagctgtaacccaagcgggaacatatgccccatctggggccaccaaaatcttaccagcttcctcagctggtggatcggttaattcacggccacagccccctggagctgggggaaaggaaaaccagggcgtctccgcaaaccagcccagagagaggtctgcggaagggcccggaagcctgcaggcccctctgcacccccaaccccaccgccatcctggacctccaagatgacctggtccaacagagtcctgcatggaaaagactggaacccagggaggagcagagccccgcccaaggtcaccggccgagcctgaatagaacccggttctccaggagccctgtctttagctgtcttgtccaaataaaatttttcaggccatcagatttccgtactccctggagtgggacttcatctgggaccaaaggagggctggtgaggggagtggcaggagggaggagtgcctcggggccccgagcaggatgagcctgaggaagagacgggtccccatgttccctttcccgctcagataatggaggtgaattgaggggagcagagacctccccaccttcagggtgggaccctgagggaccaggacacctttgctaggggatgtccctcctcactcctgcacaagttcctcaaggacaccctcgggctccgaaaacggggggagggggacgacgccccagaggcccctgagcccctggttcttcccgaccctaagggcttttctccctcggttcccaggcggcgacggcgggtagcgcgaagcagcaggcgcaggggcgctgggatggggatgtctctgcaggtctaaggttccccttgggagtctaaacaaagactacggcagcgccgtcccctcccccgggaacccgacgccgcgcggccacagggggcctggaggggcgggcagggcctcgcagcgcacccagcacagtccgcgcggcggagcgggtgagaagtcggcgggggcgcggatcgaccggggtgtcccccaggctccgcgtcgcggtccccgctcgccctcccgcccgcccaccgggcaccccagccgcgcagaaggcggaagccacgcgcgagggaccgcggtccgtccgggactagccccaggcccggcaccgccccgcgggccgagcgcccacacccgccaaacccacgcgggcacgcccccgcggcgcaccgcccccagcccggcctccgcccctgcagccgcgggcacgcggaggggctcctggctgcccgcacctgcacccgcgcgtcggcggcgccgaagccccgctccccgcctgcgcgtctgtctcgtccgcatctccgcggtgagtcggcggcgccctcgcccctgagcccagggccagcttctctcgccgccgcggctgctgcgcgcgtccccgcccagcccagcccagccccgagcacgaccccagccccacgcacgaccctagccccgcgagtcccgcaccgactcgctcccgccccatttcgcctccgcgggggcggcgccccctcctccccgcggctcccgctctccttcctcgccttcccggccgcgctggggacccccagccgccgtccgcgaccccccaccgcgacgcccggaggcggcggggtctctttgttcgggcggcgggcacgggggaccacctcccacggtgtcaccgcacccaccccgcgcccttcctccgcctcctggagttcaccgggaccaggtggcggcgggtgcctttttgggggtgcgcggccatgcaattggtggatttttttaaaccgttttggaggggggagcgcggcgttgggggcgggagagcgctcctggctgtgagctgctcctgccgcttcgctccgcgctctcctgccgctccgctccgggtctcccgcgctcctctccccggctcggccgagcgcgctgccccgacgccgccacccagagccgggccgcgccgggcgccgagatgaaggtgctgggacaccggctggagctgctcacaggtaccgcccgcctgccccgcagccggccgccactttccgagttggagcggactccgggcgcggcggccggggactggggcggctcgggtctgagcaggaaggggtgcggaccccaactaagtcctagttttgtgctacctgtttgtgtgcggagcccagccccgggagaggacttgaggttgtggcgagtccctggcgctggcgtccgggctgcgggagcaccggtcagggggtggccccatggggtctctgaccagcggagctcggattaggaccctgaaagctagctcagggctcctgccctccaatcagtgtcgcttgtcccctaagaaaggacccgtgggcttctggcaggacccgcgccatggacctcttatttctgcgccctgtgacaatctgagccgtctttctctgggggagaagtttcttgctgggagtggaggcgacgccaagtggcctgggaagtgggaagccagattggaccctactgactggggaccctcagccttggggctcctctggagaagtgatcagttgccctgctggaaactcacatccagggggcagtggctggagagcaagagcgaacggtcaggaagaggaggtgggaaagggagcagggacgggggggaggattcgaggagtgacttctgtgttctccccggtgtggagagacccagacaggaggaaaggaaagcaacccggtttcctccagctctgggacttataggtgctccatccgtgtatgtcagatgagcacagattccagtaagtgtcctccgacacctgggggagggggctgatcactgccttccaggaccttaatgtccgatgagggagcagagccccggagccctgttacaaggctggaaggggcagccgtctgtgggtgcgctcaggaaacggtggaatccgagtcgggggcagcttttgaagacctcgaaaaacaatttttgttaatgaagaaggaggtggcattatgggttcaggatcaagggaatggcccatgctctggggtgtgagagaggcttgttcggggagaggtatggaggctgaatggcctggaatgtgcgtgtgtgttgcgggggagggggctagaaggtgggggtggggtacgttgggtgaggttttattaaatcggcagtagcaggccaggcgcagtggctcatgcctgtaatcccagcactttgtgaggctgaggccagaggatcgcttaaggccaggagttcaagaccagcctgggtaacatagcgagaccccatctttacaaaaaaataaataaataaatcagcaatagctcgtgacagacttgtagagaaggaaaggtggaagctgttcctgggacactggaaactgactgggtgctcagaagttccagcgctgagcccagttagccacggggctggcaccccctccaatcccttgccaactttttctttgtggagctcacttgaggcaggcccagctcacccaacttggaagggaaccgcgcaggccactgaacttgttaaccccctggagggaaggcttgcggttccatgggtgttttgtttgtttgttttgttttgagatggagtcccactctgtcgcccaggctggagtgcagtggcacagtctcggctccctgcagcgtccgtctcccaggatcaagggattctcgtgcctcagcttctcaagtagctgggattacaggcacgcgccaccatgcatgcctaatttttgtattcttagtagagatgggtttcgccatgttggccaggctggtctcgaacttctgacctcaagtgatccacctgcccctgcctcctaaagtgctgggattacaggcgtgagccaccgcacccagcccccaccatgggtttttagagctctgcatggtgaacagatctcctggttcctctcttccccaactcttcctgcccccaaagggcctggatgagaaatgctcacaggagacatgtgtggggggctcaagcaggggtctaggctttggagaccccatgcactgttctctcatctcctggatagaggagtcaggacccctcttcccccaccagatggggtcaccagattctggcgggctttctaagtccctggccagggtggatgaggcccctctcagttactgtcatcagtcacaggctccgcccccattgtacctgcaacctggccctgggagacaacccatcccctctgttgctattttgtggtcttcacagacttccctctctgtcctcttccttttctcacagatcgggattctaactccaaccccatcagtagccagctgtgtgacttcagacaggttgcttaacctctcagacttcagccctctcatcttaaagatggaactgttggcccctgtctctaagagatggtgtaaggcttgctgagatagtgtaggcgacacacgtggttgggacctagaccctggtgggcacctcttgggccccacctgtcctggctggttggcacctagagcatttggatctgcccccaccctcctttatccctgctttcctgcgtctgtgagatttgacccatctcccccattggataacaagcaggtcacagtccgggaggtggccactttattgtggtggcacacagcaaatgctcaagggttgctgactgaggctaggctcacacagcccactcggcatctacgtctctaggataattctgtagaggcctctaactccctgccccgacatcagccttaggaaatttttactgaattttaaaaactcatattgaaggctgggcacagtggctcacacctgtaattccagcactttgggaggccaaggcaggcagatcactgaggtcaggagttcaagaccagcctggccaacgtggtgaaacccccatctctactaaaaatacaaaatcagctgggtgtggtggcgcacgcctgtagtcccagctacttgggaggctaaggcaggagaatcacttggacctgggagagggaggttgcagtgggccaagattgcaccactgcagtccagcctacgcaacagagcaagactctgtctcaaaaaaaaaaaattgatttagttctgtatacttttcacattctcctattttattatcttgcctgtcttgccagtgaagtcagtgacttcctcatgttcctatttctcagaagaggcagcactggattcagaacccagccctgagccgggagttgtcccatatggcattctaactgcctggctttgggacagtctaatagccgttccacacttcattttcctcatctgtaaaatgggatcgtcgtggtgcctacctggctgggatctttggagattaggtgtgtgagcgtgtgcaagacgtcgagtgcagcttctggcgggcggatagttgatgacagtcgtggagctggcgtcctcaggccgcctcccgagtgcactccatcactgtgctgggggctccttctcctcccactgtgctccagtttccagattccctccccgcagggtgttccagttccttcgttgggttctcacaacagtccaggcctctctgagctgtgtctctgggggaccctacttaggtctcaggttcctgtgagtcaggcatggcctcagggggtggggcccttccagccagacacccacagctctgacatcatggggaaggccaggtgccccctcacatgtgggactagaaggagacccatgggttgtcatgcccagatccagcatgtctttttgttctggagagaagaggaggctgttggggtccttctggaggctttgtaaatcatgaatgcttgtcgaggccatttccaggcgcctcagcggggtcttgctgcaacagggacaaggaaggcgggggttttggaatcagaggatgtgaatttcggggtctcccctgtgcttctcagctcctgggcaaattgttaccttctttcggcctcagtcttatcagtaaaatgggaataatagccctcattaacagtgttaggccagagcagttgtctcacgcctgtaatcccagtactttgggaggccca

SEQ ID NO: 87 - DNA, 1587 bp, Homo sapiens
ggatgggttgtagataagagcagttgagaaccacgcttcatctccctccttagaaactctgaaacgaggctttattcctaccagaagttcagattgcattatggtcatctcaattccaaaatgttagatggcaagaatatctgcccatccttcactttccttggaaaaagttgctcttcgggttttatatgcgattgcagttttccagtgtgtgaaactaggaaaacaaaacactcaacggtgtacatccctacacctaaatagtcagaaataataggcagctaggctaattatccttgattagcaagatcagagccattagggtgctcactggtttaacaaatgaatgcccttaggcgtctatcatttgtaactcctagaagctttaatttccacaagaaacaaaataagaggggccttctgcttttaacagtgaaaagatcgttctccctcccctctccacccgggtcaactcttccagccgctccctcctgcatcacgaacacacgctgcaggaaagcgcatttacagcccgggacatccccagacctcctctccaaaattccccacctcctgtgcataggagaaactgagagaagccctcacttcctttccaaacttcacaagcaggggagggagctgtagcagactttcacctccgttcccaaaagcgaatgtgaaaaagtccgagaaggcacgtcctgcgagtggaggttaaaccgaaatctgaacagaatgcacggtccccgcaaactacgattgataaagaagatactgagacgtttgcgggggatataagccatggttgtctcgccttcctcccctccctgccaactatgtttcttggagaaatcgccggttcgattcacgcacacatttttgtaaaacacggacaaaaccataagtagttaccttcattgttccgtcggccacgagggaagctcgagctgagcggagggcagatcccaagggtcgtagcccctggccgtgtggaccgggtctgcggctgcagagcgcggtcccggctgcagcaagacctggggcagtgcccgaggcggcggcgagtacacgtggcgggctggattgcagaccggccctctcgcggcggagactcgcgacctagcggattgcatcagcaggaagacactaaggctgctcccccaggccgcccccagatggtggagtctctcccagcccgaagattcggagccagcgcccagacccgagcctcactcactgctcactcccggggtgcagggcagaggtgccagtgttgcaagcaaatgacacggttacccccgaatcagccactgtgggtgcgtatccgagtgtggggatgcccgtgtaacatttatatggagacgtcaaggaggaggaaataaacagatcagaggtcaaatgtgattgccattccgtcatcactggctcctgcccacctccctactgtccccaaagtaactttgctgcatgctgagaggaccacggcacaatcctgcccaaaagtatacatgtatcccccgcggctactttaaatgtacttttgcagtagtcaagaacatgtgcctggtttgccgatctctttcccag

SEQ ID NO: 88 - DNA, 3381 bp, Homo sapiens
ggtaaatcaatggacctttcactaccttccccttctctgggctgtagcagtgagttaactggctcagacttgcccagcggaatgcccttcaggacacactgagtactgtagtaagagctgcatatgcgctcagggagaatgtggatagacgctgtttgtgattcacgggtaaagggccttctcccagaggcagcagaggcagatcctgtctctggtgtgccctggcccccgacttggaggcctcctgggccaggccaagaccttccccggcagcgatggtctccagccacactcaactgccctgaagggacatttcctgcttattcccttgcccggctgtgtcctccacccggaaggcctgtgccttcttcgcctgcatgtcctaccctgaggaggctcccttggtctttcatcgctctccctatgggtcttcacgccttcccgaaccaccgcgcccaagcaggagcacgttctcggccctcttcacagggcgctcctcctcacaggggtgcccgggatttttattctgtgccttcctggtggctcctacaagtctggaagggcaggaggcgcatctcactcctctgggtcccctcccctagcgcctggcgggagcccaggctgcatttgtggaattcatgactttttctctcctgctcaagctgaacacattgctggctcctgctcgggtggagcccggctaattagagtgaggggctccccgtagggcgaaggggtgcgctgtcagatgtggcattcccgttttacggagacacacggtgtcttacacgccagggagaggtctgagacgcaaagagccgtcgagcgggctgcgggattgcttcgctgtcacctccgcctgcagccacccttccgcacgcacttgtgtgtgcacccaggccaacatggaaggcgccatcctaacttctgccgtgagcaggtgggagggaagagagacgagaggtattccattggttgtctgggaaaatgaattgcaccttcccctcccttgcggaggatcaacttttcccaccccctcgggtgggcactcgcatcctggggccggagcctgaacccgggagccaaggggccccagttccagggacgtgaagctgagcgtacagcgggcgctcccagacactggggaaagtgctttacgatgtcccgagtccctccagtctcgccagcggggcgagcgtgagggtgccccgaccgaccagcggccccgggtgcagggtggcgggcccggcggcgcgcgtccccctccccctcctggcggcccgcacgtgtcgcccgcgccgcgcccccacgggttacgcgcgggtcccgcagcgccgcggccgagccgggctgcccggcccgcggacacagcgccggccgccgcatcccgtgcggggccgcggcgcgatgctgcgctggaatgaggaagcgcggcggcgaggggagggcccgggcgcggtgcgcgcgggggtggcggcggcgcgccgagcgggcccggcgcgggcgagcgggctgcagccggcggcggcgccagcaggtacggcccgcacccgccgccgccccggcggcctttgggggctgagccggagcccggcgcgattgcaaagttttcgtgcgcggcccctctggcccggagttgcggctgagacgcgcgccgcgcgagccgggggactcggcgacggggcggggacgggacgacgcaccctctccgtgtcccgctctgcgcccttctgcgcgccccgctccctgtaccggagcagcgatccgggaggcggccgagaggtgcgcgcggggccgagccggctgcggggcaggtcgagcagggaccgccagcgtgcgtcaccccaaagtttgcggggtggcagggcgcgcgctctggccacccgccgctctgggcggcagctggtggcaacgcaagggcgcggcgggggcggccggcgcggagggggccaggtacggggcccgcgggcggcgctgtgcgcgcggggcagccggtcggccgggagcgcgaaagcctggtctgagccggctgggggcggggagtgtggcggagaaatggggaacaatgcgagtgagcaacttcaggaagtcattgtgaaagaaagctgggaagagctccgcggccaagttagcaggacactctaacaagtgactgcgcggcccgcgcccggggcggtgactgcggcaagccccctgggtccccgcgcggcgcatcccagcctgggcgggacgctcggccgcggcgaggcgggcaagcctggcagggcagagggagccccggctccgaggttgctcttcgcacccgaggatcagtcttggccccaaagcgcgacgcacaaatccacgtgagtgttttcaaattgaatttcaataggaaaacttggggtaactggtgaatttaaaaaaaaaaaaacacagtaaagaaaagcggtaaggttggtagaccctggtgtcgctcaggtccgcctctcttttctgaggacagtgagagagttcacttctgtcaagcgtctgttgctctgcactgtgccagcaggtgcaggaccaggccgacatgggacacttctgagcagccccgctgtcaccaggagaggagttctagctcccaaccatatttaaatttatgtagacctacatatacccacggaagtcagcctttataaagtcgtgtgtaaagagttttccttatatttgagccgggagctttctttttatactataaatatgatgagatcgagtctgaacttaatttctgcaagagaggaattatcccggctttgaaaagttagtccttttgctgaccgcaggtttgacgctcaagtcaccaaaccttctcaggaaaacccttagtaatattaaggcatcaggttacttgcggttatatttgaaatgtattttaaatatttgtcaagcatcgctgctgatgcctaaggaacctcgtgagggcttgtttttccttctaatttggaggcatctaatgaccgaaaaccgtagcgattccatagggtctgaccaggcacagctttcaaatgcagcttccctctctctagggactgcagcccacccagactgaatttcaatgcggtgcgctttgcttaggttacccactcacaatttcccactgcgccgcaggcagtatatttcagctttgagataccttgttttaaaattccagacaaaatggtgttgaggaaatgtctccttactagtcccatcaacttctgttaaaagaggaaaatttatggaatttgaaaatactgcgtatgatatttaaactttcatagacattcaaatgcttttaaggccaggttcaatttggttatgagtcgaggggtgggggggacccacatagaaatgtcctgggtcctcttgagtttattt

SEQ ID NO: 89 - DNA, 7398 bp, Homo sapiens
ctggagccggtgggctctgagcacagtcacgaaggcatgcgcactttctggctgctctcctgccattgtcccttgggttcccattttggtgaggcgtgcaccctggcggcgctccccagggaactaaatatgtttgctgcactgtgcgtggagatggagaatgtacaattggctgaccctgtgctaatctggtggaactccatgccagccctgggaaagaacaattgcatgggtgtgtccacactcaccaggtgctttttagaaaaacactcgagaataatgctgtggcttaggatggctgttgtgccggacccggcatcttcccaggggggctgtgttgttgggctgagtttcttaggtactggacccccaaatccccaaatacggcgtggacaggtggcccagtaggggctggactatccgataggcccaggtgctggagttcagacaagacataccctggcctggcgtggaagatacggggtgctattaatggcagcaatggctgcatttctgaaacccgggctcccaggccgacgagggtgtgcacgcatctgaaatgtctgtggttttgcagttcccatgtccacaaactcacttggttgaaaatagttcaaaatatccaaagcatgagggagggagtgcctgcttttcttaaaaaggaaggacttgatttcatctacttaaaaagccacccaaacctagaacattttccgcaagagaccccctgccccccgcctctccagaatggctggagagtctcagcactcctgcacatttgggatatttcagagggggtggggaggggcaagtgggcagcgagcgacctcagacccaggatgagctgtcaggcgctccccggccacacattcaagggaccggagtgcagttgtagcgttgcggcctgctgcttcgggggtgggggtgttgttccatgctgtgaattctcacatggcccctgactctgggcagaggccgagggtctaagggacggggtgacagggagagcatgcaggagtgggtttctggctttccagggcgagtggaagaagcgcctctctctcttgtaggtgacagacctggggggcccttcttgaggatgagagcctgttgcttctcaagttctgtgtctaacccaggtccccaggtctaccccagcccctcggccctgcctgccttgtggatgatatagtttaagggtagagaccgctggcctggagggaaggctaggcctcaggttagggcccagaagggagggagaagcccttggggcagctccctttctgctcactcactgcctagctccttccttcacaccttccttcggaaacgtctgctcctgacaaggtctacttcctgctctcaggaggcccttattgtggaggaagggaggcgtcgcccgtccctggcttctctgacagccgtgttccatccccgccctgtgccccttctcccggacagtgccttctccagggctcacccaggagggtgcagcggtggcccccggggcggtggtcgtggtgggggtgttagctgcaggggtgccctcggtgggtgggagttggtggcctctcgctggtgccatgggactcgcatgttcgccctgcgcccctcggctcttgagcccacaggccgggatcctgcctgccagccgcgtgcgctgccgtttaacccttgcaggcgcagagcgcgcggcggcggtgacagagaactttgtttggctgcccaaatacagcctcctgcagaaggaccctgcgcccggggaaggggaggaatctcttcccctctgggcgcccgccctcctcgccatggcccggcctccacatccgcccacatctggccgcagcggggcgcccggggggaggggctgaggccgcgtctctcgccgtcccctgggcgcgggccaggcggggaggaggggggcgctccggtcgtgtgcccaggactgtcccccagcggccactcgggccccagccccccaggcctggccttgacaggcgggcggagcagccagtgcgagacagggaggccggtgcgggtgcgggaacctgatccgcccgggaggcgggggcggggcgggggcgcagcgcgcggggaggggccggcgcccgccttcctcccccattcattcagctgagccagggggcctaggggctcctccggcggctagctctgcactgcaggagcgcgggcgcggcgccccagccagcgcgcagggcccgggccccgccgggggcgcttcctcgccgctgccctccgcgcgacccgctgcccaccagccatcatgtcggaccccgcggtcaacgcgcagctggatgggatcatttcggacttcgaaggtgggtgctgggctggctgctgcggccgcggacgtgctggagaggaccctgcgggtgggcctggcgcgggacgggggtgcgctgaggggagacgggagtgcgctgaggggagacgggacccctaatccaggcgccctcccgctgagagcgccgcgcgcccccggccccgtgcccgcgccgcctacgtgggggaccctgttaggggcacccgcgtagaccctgcgcgccctcacaggaccctgtgctcgttctgcgcactgccgcctgggtttccttccttttattgttgtttgtgtttgccaagcgacagcgacctcctcgagggctcgcgaggctgcctcggaactctccaggacgcacagtttcactctgggaaatccatcggtcccctccctttggctctccccggcggctctcgggccccgcttggacccggcaacgggatagggaggtcgttcctcacctccgactgagtggacagccgcgtcctgctcgggtggacagccctcccctcccccacgccagtttcggggccgccaagttgtgcagcccgtgggccgggagcaccgaacggacacagcccaggtcgtggcagggtctagagtgggatgtcccatggcccccatccaggcctggggatatcctcatccgcctcccagaatcgggccgtgggggacagaaggggcctgcgtgcgggcagggagagtattttggctctctcctgtcttcggggtttacaaagtgtgttgggacttgcggggctgctctgtccaagcctgggtctggcgtccgcgtctctgagcctgtgagtgcgtgcgctttcctgcgtcctcttgactgccggtgctggggctctgcgtcctgcgtccgcgggagtaaatacagcaggcgaaggggaagctcacacaatggtctccagcgctctggggcagggcttctgaggggcgggcctgcctctgccgggacctggagcccccgcccctcggagaggctcctaggctgacttgggcagagccctctggtgggccgggagggggaaaggctgtgttgaaatgagcaaactgtccaggtgtcaggccaagctgggaggtgaccagcctgaggtcctccccgctccatggccagaaccagggctgacatctgggtgtcctgagcccagctgcccacacggcccacctggggtcagccctatctgagtgggggaggcggggcctcctgggggaccagaactttggctggacgccaagcagagtgccagtggctgttcttcagggctgggcctgaggagggtgtggggcggcgaagggacgggagggggttgtgatccagtggccactggcgctgtgcagagtgtgagctggaaacatcgtagttactttgtcagcttagtggtgaaagccctttttcaggctctatccctttgcatccctgcttcccagagggaggggaggtctgggtctgcagagctgggagggcttgctgttcccgcccccctcccccacaacacctcctcatctggacatctttgggcacatgctcatactggggtctccctaggtccactgtgttccgttgagcctcctgcagtccccgagtgaatgtgacctccctgcccctgcctctttgcaactcctccctgcgaccgctcctccaggggccttccttgtcccaaatgtccaagtggcacgacttagccggtctgaccactttccagtaagcccttatggagagaggccctgtgttgtgcagagctctcctcctgcctgcgggatcgaggtctctgctctcagttcctaacagaaagtgtcgggcccccagtgggatttctggggaagaactctcgtgtctcaacgggagccctgtggcgggaggggaggccagggtttggggttgtgttcgttgtacagctgtcaccatttgcactatgaaagttgttagtgccccttccttgggtctctgggtgtaactccacccttgcccccatgtgcctccatctggagctgcttctgcggctgtctcccaagccagttttgtgaccctgtaatttagtccaagacaatgggctcattgagaccatcctggtgcagcagttggcaatcctttggctctgggggaaggtttctcagtctcggggagtggggcctcaatctgctggttccctgtgtttatcagtctcccccttgtgtgtcctgaatggttttgctgggaattctggtcttagagccatcaggtggcccgagtcgataggcgtgagagagtgtgtgtgtgcatgagtgcgcatgtgcatgggggctgacctggggtatggaaaggtggccctccctggtgcccaaggagcctggagtatagttggagggtgtgggggtgtgtatatgggagttggacaaccttgggtggacagacagacgtggggaagggatgattgaaggaggtggaggagagagtgtgattcagcccagccaggggtgatgtggacaggcagcttccgaatcagggtagagaaaagtcaccactagctagcaggggagaagtcagtatggaggaggcggaccttgagggagagtaggaattggattgcaagaggaaggagagccttctggccagcagcagccagcagcagtgggggaggctggaatgagctggctggagagggggctggggcataaggaggggcctgcctgtgaagatcatatgggccaggctgcggagggccaggcatgcccgccgggagtgcagctggtccacgggaagcatctggagtggctgggaatgggcgcaggagcagcgccgtgggagcacaggtctctttcccggggcggctcacctggtgtcttggttcctgcaaggtaggccgaaagggtggggaggaaactgccagctccttacagcgctgggatggtggccccagggttcctgaggccagcggatgtgggtgcctgtcaccatgtgggttgctgaggggcggagactccaggggccaccccaaagcaggacgagctctgagccacggcatctctgggggcagttttccaatcgagcagacgtctaggcctggaatcctgtaacagaggccacagggccctgatcagggtgttctgggaggcttagaactagtggcagtatacagggtagacggcaagtgacctggcatggggaaagaggcaggtgcccaggccggcacagcacacccgtaaggaacaggtagacgggaagccgtccgtgggcctgtgtgtgtgctcggagttaaaattctgccaatgtcccatgtcctgggcacatctaccccctccctggggagcacctttctcttcaccttttccttccccgcctgtccccttcacccagggccttccccatccctcccgtcttggggaccgagggcaccatggctttatgttccatcaccgatgagttgcacagggattcagtccttccgctgttcaggccgggtccttcaggctcagggttcccaggaatggagagggtatcagtgtcttccatggactcaaacttcccgcatcccgcctttgctcccccttcaagataggtctccgagcttcaaggttttagggctctgtggaggccgccacgtagcagcaaggagaatgttttgtatttggctgatgagatttttagagtctcattttctacttttccactgtacaaacgggcctccaggcgactgcagcacccgccactgcccgtaatagggtgacaagagggatgaccctttcctctttctcccttctggttggtggaggcacggggctggcggacggcatgtgctttcgtgaattcaggccaaatctgttatcgcaaacacgattacaactcgggtctttgtgtaacaaaagcctttccaagtaccagctgttggcctgctttgctcagcggtgtttgctgtaaccagatttgcacaccgagaaagaatccaaaagtccttgatgtttgttgaaacaatctggcccaggacccacgtgctcagatcccagagctgtgtggcatctgagcttctcccgagcccccactgtcgcccgaggaggacccccagatctgtgttctggaggcagagcaggctgtgggacgggcttctgggtgggaaggaccatgtggatatgccttcttgtctgagagtcccaacacctccgggacgtgggagctggcgcgctggcaggattcaggtgcctctttcctctctagagaaaaaggccccgttgcttggtaataggtgcagacctgtccctaattaatgccagtaggcctcttgcgtgatgaaccctgcctttcagccaagactcaaggcatcctgtgaatactgcctctctgcagtttgagcttttgtggtgggaggcaggagccatggggagtgggggcaggcctcttacacgggtcccacagccactggcagcactgacttgatgctctttgagttcagagcccagggccagacagacccactgtcccgactacgagttggttcatttagaggggggcggacacagcacccaggcagcagatgcactgtgatcagccttgcagcggggctgtgggttctctgggctggatgtccgggaagaggcaggtggaggtaaacgccaggacacccctgcagtgactgggtgactgcaggctggaaatgctttctgtgggctgtggctgtccaggaaggttttgaatggggctagtggacagagtttgcatccagaggggcagtgctttggaggagtgaggggtatggcagtgtagggatgcccaggccgccctcactctgccattggaaagctgggcggcttcggtttcttcagtttcttcgcctgtttccctgcctgcaaagtggggttagaaacagctctctttgagggttgctgggggactctgagatgcagcccatggcgctgagcacgggtcctgcctcctacgggtgtggtgggtgtcgcggctggtgtggcatctgggcgggaaaagggggcatttgcaaaggacaggtaggtttggatgcttaaatatgcagatctggggatgggaggtctcaggcaagggcctgtgtgatgccactgta

SEQ ID NO: 90 - DNA, 2396 bp, Homo sapiens
gaacacttcacattgcttgaaagcatcttacactttttttttccctcaactcacaaagcagtttctttctactggtcgaattctcaaggcagaaaagctacatacgtctctcgtttcttcactaattgttctctagaaaagggaaagtgaagaagggaaagagaaaagacaacggggaagaaaagagcatagaggagagaggaaaagtggggagagaaaggaagaaaaggactgagaaaacgcaggagccctggcttgccggtgagcagagccggcgcagccacagcgcggagccgcggcgcccactggtcctcggagctgccaatcggcgtgtaatcctgtaggaatttctcccgggtttatctgggagtcacactgccgcctcctctccccagtcgcccaggggagcccggagaagcaggctcaggagggagggagccagaggaaaagaagaggaggagaaggaggaggacccggggagggaggcgcggcgcgggaggaggaggggcgcagccgcggagccagtggccccgcttggacgcgctgctctccagatacccccggagctccagccgcgcggatcgcgcgctcccgccgctctgcccctaaacttctgccgtagctccctttcaagccagcgaatttattccttaaaaccagaaactgaacctcggcacgggaaaggagtccgcggaggagcaaaaccacagcagagcaagaagagcttcagagagcagccttcccggagcaccaactccgtgtcgggagtgcagaaaccaacaagtgagagggcgccgcgttcccggggcgcagctgcgggcggcgggagcaggcgcaggaggaggaagcgagcgcccccgagccccgagcccgagtccccgagcctgagccgcaatcgctgcggtactctgctccggattcgtgtgcgcgggctgcgccgagcgctgggcaggaggcttcgttttgccctggttgcaagcagcggctgggagcagccggtccctggggaatatgcggcgcgcgtggatcctgctcaccttgggcttggtggcctgcgtgtcggcggagtcggtgagtgggccaggcggaggatgcgcgcgccgtttagggtgtttgaagctacgagaggagcccgcagggaataggggagcgccacctggggaacccccagtccccaagtatacaccggagatccgctgggacaaatgcgctcgtccggtcaccctttccccctcttcccttcctcagaaaagcgctgctcgctggcgttaccccgcggtccgcgggaatgggggcaccgagaattgcggtttggtctagccgcagaggcccctgaagtcactcccaacttcttcgccctcggcgggtcttgctgcgtggtctgggaaggacggaggggaaagggtggcaggaggggggagcctgggtcgggcccgcgagggaacggctccactccgcgcgctcctcgagaccagggatgacctggaaacttcggggtcccttcctccgcacaccatcccccccgcgccagctttcctgtttgactgcatgcaagttctggggagatgggggccagatttaagagacccgcgagtgtccagagagaaaagtttgcaaaagttcttttgtttgatgctccctgcggctagggcgaggtaaccgacactacgtggaatcgcagtaggcgatccctcaaggggatactgggggaggcacggaacgcgtccgaaaatgctgggacgccggccactggattcccagtcctgcggcgaccccctcctcgttgaggggtggaggttgcaccgcggggcgtcagggacgggaggacattttcataggagttacacgggagtgccgcaagcagggcgaggcggggtacgtgtgacacggcgctcggcttcgggtcgcctggccgctgggggacagaggcttccctcccgccacgctcgccctctctggccctggcggggcgcttctggggccgggaggagtctcgtctccggcggagcgcctgccggcacccagcttccctcccccgccctggcggtgggaacttgatttctccttttggtcgcgcttcgggggctggagcttgtttccccacgtcgcccaatgagcgccctctaaagggaactgcctccttggcctcctctcgtccgcagctgcctccacctgggcgccaggagctctgtcgggccaggtggaagcttgagcaccccagatttcgtctgcagcctcagtgccctctggggtctcagggagtgcggctgtttctggcccttctggttccccacgtcctctccctttgccatttaataacgtgtcaatttctaattaattgaatggtcttttcaaagacaaattatatcatcttaaggtcttttaggtagttttat

SEQ ID NO: 91 - DNA, 3696 bp, Homo sapiens
tgaaccgagtttaacatccttctgtacaaaaattttccgacaggacaaataaactactcttccactgaactgtgacttttgagctgactctagggtgcacccaaccttccagaggtgtctggcttaaccctcaaagaggcagctccagccccaggagccgaccggcaccacagcacctgagcagggcactgcaggaaggaaagcggaatctctagtgaaacttctcatttcctgtcgctgctgagagaggctggactcgctccttctcacatggcttaggaagagctgtaaacgggagcttgccggctggccaccagctcaggggccctgggggcggcctccgggttctgctccctgttcttccctttgaccaatgtcactcctggacagataacgggaccaaacgcccttcacttcctcgagtcctcatgtatgtcatggttcctcttttaggaaaacatgagtacaagacgcaaagcaaaagaagagactaaaggcaaattaccccatcacctcgtttcgtccctccccttcgttattatagaagagcttgatcaaattccctttgtcgcgccacacacacgcagagtaggtgaagggcaccctaagacaacttatttctttcccgcctcacagaaagcctttacgaaatcctcacaccatctccggacgcaaagctttcgcattcagcttgaggagctaaaccatttcaagccaaggtaggaaacgccaaagtggtgccgaagtggtcccaaagcagaaggctgggaagcagggcaagctcagcgcacctagacgtttgcatttacacaaagaaattagccgcatgattaatgggagctgccggctggaggcggggcgcccgtgccggcctcctcacctgggacatctgcggcctcaggttgatctccttcaggttgatggagtgcgcccggaggttgttaagcagctggcagagcaggactccatcgcggagggtctgcgcaaggtcgaacacctgagccgagtcccaggtcacccggtggttggtgggcagcaccttgcaatggatgagccactgcgcgcactgcttccacggctccatgcccgacggctccgggacgcggctgggccggggcgggcggcaaggatgcggccgccgccgccgccgccgcggttcctccgcgccccgccgacgccaacagccgccggccctttccccgcgcgggatcgagggagcaggagccgcggctgacgggtcgcgggcgccgcgctaggctcggctccggtcccggcccgggtgcgccgcgacccggccgccgctgcagcgagtcccgcgcgctctccgtgcgccccggccggctcggcggcggctgccgcgcacaggcttccgactccagcgcccggcccgccactgagcatgcccagcacgccggccggtctcgctgcggtccgcaagtccccagacgcgcgggtgggagcgcgccggcggccggggctggggtctgtggccgagggcggggcgcgggggaggggccggcggaggggggcggcggccagaaaggggatcccgcgcccccgcctgcagccttgcggggctcacgcagcccccggcgtcctggggtcttctctcggggcggcttcccggctttgcggggagtgtggctgaatactgtaatacgatggggtcccccaggaccccctaaacaacaaatgctcaaaggagcgacggattaattggggcacccagactccccagagcaatgaaaaaagtgcctagagcatcagtagagcacggcgctgaaagttttagagatcgtcgccccctcgccccttgcagctctatcccctccattctctatagctgattcctcagcctattcctcctagttgcccctagtggtgttttggcaccctcaaagtgagtgagagtgcgtgtgtggagacgcctgcggaaaccgccccgatccctgagcctatttcctcgcgaggtgattttcacttggagctggtttgcccctgcactgtcaggctcggaactgtttgccgttgctgttctggcccttttgctgaccccacaaaaacctgcttgagaaaggcctgtgccacggtgctagactgcgcatgcgtcggcgactggcggccgggtttgagagcaaagcgcgttagccctgggcagctcctgccgggctgttctgggatccttagtgaaagttggaacttgaccccagaacttttgcgcagtgcacaagcagtgcattcgtgtttcttaagtaaatcttttggagactctaaaccctaccccttttactcccactgcacactgcccctcaaaaccacaattaatgtttaaattatttgtggcaagatattgccacagtgaattattgctgttcgataggaatgtggtccttcgtcacccttttgagatgcattcacttaaaattagaaaataggccgggcgcggtggctcacgcctgtaatcccagcactttgggaggccgaggcgggcggatcacgaggtcaggagatcgagaccatcctggctaacacggtgaaaacccgtctctactaaaaatacaaaaaattggccgggcgtggtggcacgcacctgtagtcccagctgctcgggaggctgaggtaggagaatcgcttgaacccatgaggcggaggttgcggtgagctgagatccagccactgcactccagcctggacgacagagccagactctgtctcaaaaaacaaacaaacaaacaaacaaaaagatgggagtttggataatttaattgtattatatttctgtagcccaaattttttattacgaaccatgctttataagcaggccaaaatattatttttaaaagaatgatctgttcaggatagtgattgcgtcctttttcgtttggctgtgacaaggatagcacactgtttacactcagcgctgaacaataaatcagcctgaggatttcctttcctcacacgcatccttccctagcaatttcctcttgtgttcaatgcggattccatcaatagcacgacattttctaggccataattttggcaaataaatcatactaatctctacatacctttagaagcaaaatccaacacatttgaatcaccatttaacacagtctttcataaagagaaattaaaaagaaatttcattaaacgggtcaaaatttaaactaaaactatgaatttttttcaggtagcatcagatcacctttggatgttggagaatatttccaaaatccacattatatacactatcccaggacgcaaatctggatttagagcccgagctgtactgctttctagcaacatgaccttggataaattctctacagttcagtttccatagatacgaaataagaaatttttttttttttttgtggggggtggacggagtctcgctctgtcgctcaggctggagtgcagtggcgcgatctcagctcactgcaagctccacttcccgggttcacgccattctcctacctcagcctcccaagtagctaggactacaggcgccggccgccacgcccggctaattttttttgtatttttagtacagacagggtttcaccgtgttagccagaatggtctagatcttctgacctcgtgatccgcccgcctcggcctcccaaaatgctaggattacaggcgttagccgccgtgcctggccataagaagtaatttctaactaatagggttgtt

SEQ ID NO: 92 - DNA, 4199 bp, Homo sapiens
tgctaaaactagaacagaccacacctaggggacaatacccaggcatgttaacggagtttaaaatgccaaggaaattacaccacaattctgcccagtatactacaggctgtcaaaccgaaatgctatgccagctaggagtgcagcaactcccatcctctggccctatttaattaggaagcttcagcagagcgaagcctgccaagcgttcgccgtcagaatctgaaggaacccgagcgagcaagaagagtgcctgacccactccacagaagcctgtccagaaatggaggagtcagcgcccactgaagtcggttccgccctcggctcgcctacatggagcctgaccagcctcagtcatgcccactccggcctgggagacccgcaaagtgttctttttctcaactcccctgtactaccttgaagcttagggaagcaaagagaggggcatatctggactgcaaaaccaatgtcttttgccgcctaggagagaagggaatgagagagagagagagatagatagatagagagagagagagagagagagagagagagagagagagagagagagaaattctattgaaacccagctcctctagaatctgtgtgacctggtcttcaacgggagaccagtgcgacctcatggcacctttgccaggaatcagcgattcccctgcagtcaccatttgatttattgctttctcgctcattctttctcataaagttatttcttcctcatcctagtaagacttttttctttaatgatgacaaagcttctgtttcagtgtttcccctaggattggtgctctttcaaaacagtgaacccagaaaaccatcccgtttaatatttctcaaaatcctcgcagctccaatgtaagcgcaagcatgcaaaggtttcctgctacacctgcactttctgcccatcccagaaccacccctcacccccgggcctgcaacagttccccttgtttctctggatagaggtgggtggtattaggggtctagggcagtaggaggtgaggggctgaggaggcgcgctagggtaggctggtctgtgctggatacgcgtgttcttctgcggagttaaagggtcggggacgggggttctggacttaccagagcaattccagccggtgggcgtttggcagtcacttaaggaggtagggaaagcagcgagcttcaccgggcgggctacgatgagtagcatgacgggcagcagcagcagccagcaaaagccctcgcaaagtgtccagctgctgcactgccgcggggactcccacagcaccatgactagttcgtgcaactctgcagcagcaaacggcttccgaggaacacaggatcgcgggggccgggcagcgggctactgagcatcccgcggacggcggcagcagaggcggcggcggtggcagtggcacccggcggggaagcagcagccaaacccgcgcatgatctcgagagtttcagcaacatccagggactgggctcagccccggagcgagagggtcgtccgctgagaagctgcgccggagacgcgggaagctgctgccataaggagggagctctgggaagccggaggacaggaggagacgggagtccaggggcagacgagtggagcccgaggaggcagggtggagggagagtcaaggcgccccgcagcccggcagccgcctctcgagctctgccgcccgcatccctctggcgtttgggaagcagcaggtcctcagcccgcccggggtcacgtgggaagaggcagtcgggctctgattggtggagcaggatgcaggtcccgggagggaggggtcgacgaggaggtgcaaggatgcaaggaggaggcggccgcggaagccacagatgggctcgctcgccaggcgctggcccgagtggggctaggcggggatggctcaaatgagaagctcgggcttcagggtgggctacccgcacactcatataccattcgcctcactctccgctccaggacgccccctaccgaaggcggggtccggactagcgcccctcttccgcgcgtgaccccgggccgcgagtgcgggccgcggctgggtggcgtctctccgagctggagatggtgggggcggaggtgtcagaggagcagcagcagcagggcagagaggggcgagtcggcgcgggagagggcgtcctgctggcgaccggcgctccagcgtgcgggagcgcgccgcctaggctgtagggggatgcaggctgggaatgtcgcggcggagaggccagggacgtttctctagggatttacaggaaagagggtgagaggcgatggtgttagaaccgctcttgccgacctggaagcaacagcagcatctcccacaagagcgtgcaaccccaaggctgctcgccgaggcagctcagccatcccggcaggcgctctccttccttctctcttctcccctctctcctcccaggccccccgcagctccgacccagcccaagcgttcgcaggtttgaatccctctcctcatcacccgctcctctccagcccgtagcctattagtgtgtccacctgggaggtgcggtcagatgtgtttggaaggtcagattggtcgggacaagtggtctgagagaaagagaaaggctcctctgcatacgccgcgggtgggttgccgggagcatcggccgggcagcggcgtccgggaaggggagagcgggctccatttgttggcccaggcagtgaccctgcgttccttactcgggtctttgccggatggccggtgacctggggcgacgagagaaggtctaactcggcaggagtctctggctctgcgcgtttctttcattctctccagcgggaagggcaaacggcatagcgggacccgccttccgtctgctgcattcttcaggcagttagacacactctttagcctaatggaattttagtcgccagtaacgggaccaagagctttcggggacaagggtggagaggaacatctttcttccatgaccggggtcactattgcagtctcagtgttttggatgccccatagggaagagctttctttttggtgtgtgattattcagtgattcctgtttttgtttttgtttatcttctctccgctctccttctctattcctctctgttatccctcctccttcttctccccccgctttcaaaagcctccggatcctccctttttctatttaaattctccttttgtgcccctctttctgtgtcccctgaatttaggagagcatttgataacatttaacaggcaattagtgtccattcccaatcacttaaaagaggcattcatatactttgaaaacgggactatctatcctttgcagacaccagcagaaaaacaaattgtacccgagtaatccttttaagtactttaacctccaacctcctcccacttccttgctttttaacttctcctttgagagatgtgatcgtgcagcacctcagtgcctcaacgaaatctttttttttttcctgtgtgaaatccatccctttatcttacatctccgcctccgtccgagactgtccctctcccctcccacctccaaagatttctgaatctcagtgtctctcactcctggcaattaagcagcagatcccagcattctagtcggtggcatctcgctcctcaccgacgaagactccattaaaacagatcaattagaccagacgttggaggcatcagaaaatcggcttctagacagagcagctaaattctttaaggaaacagaatacccattagatagagctgccaactaatattgcaaaacaaggaattagaaatttctttcgctacaggctttcagcagagaaggcaacataaatatagatcaagatttaacaactctacagcagagaatgagaacatgtcattttccatagcaaggctggtgtggtaactaatcaggcttatgaaaataagtcatgcttgaaactaaaggcaaagtccttaaaagtgtttatgcagtaattatgataatgaaacaggacctgctaggatttcagagtttggctatgtaagtagaattttagagaacctcttagcagaggaaaactgtttttgaattttctgctaagtaaatttttggcatactttctaataatatatgctcttcctaagacgttttgccaaaagtaagttaaaactccaaaggagttaattactggttgtaactggttaacaaatgcggttgcttccacagaggtcctttaaattatt

SEQ ID NO: 93 - DNA, 5716 bp, Homo sapiens
tgtgtgtgtgtccacggcgcgggccggagcactcaccatctcgccgggggagcgaggccacttcggggtcggattggaaatgttgaggcttcgcttgcttcctccgcgacatgctggctcaaacatcagctggggcagaataaaaaattactaaaaaaaaatcttctcaaaattacggaaatcgagcggcggcggcggcggcggctccccccgcccgccggcccgcccgccccctccccggctccccggccccgggcgcagcgcgcatgtgtcctgctataattatgattatcaataatgcattgcgattaatcatagaggggctctttgaaaggcgattggcaccgggccagcgctattcaaacccgctcgccttaatcaattagttcgtgatttgctgcagacccctgtctctccgcgcgctggcccaataagccggccgcggggctggctctgcgcgccgcgccgcccgacactgggttaaccctctttgcgaccgcccgggactccgcggcccggccgccgggggccggcctcctctccactgcgggcccggcgccccggcaggcgccccccgctctctccctctcccctccctcttccctccctccttcgcccccccccacaatcccgggctgggccgacccaaattagcatgccctccccggaattgagccgccccgggtggggggtggggccgggcggcgagggctggggacccgggctgccggcgcgctgcgaacttcccaactcgggggcgaggcgcgccggcccgcggggggagggagtcccgcggcgggatcaggggggagggcgtcccgtttcgtgagtgtttcttcgtttctttaagagctaccagaaagcagcccacccccacccccattaaaaaaaaaaataagcgagtggggaagagccagccccacgccggtccgcctcccgcccacggctcggcctccctcccgcctggctcggcgctcctccgccccgagttctccatcccctggatcccgggctcgcgggggcgcggagaagaggatccaggaaaggtttgggaacctgatgggtttgtggctggggggaggggcagttctcccgccaccccatcccctccccgccaggggcactgatcctccagcttcttgcttgctccctcgctccctttctctacctcggacattcccaggacaattagggctgaagttttcgggagaagcagccccgagcccgtgggataagggggcggcccggctccgcccaggacccctcccggacctcccccccggctgccccggttggctgcagggcgcgtcactccgcggggaggcggcggcagcggtggcgccgcagagccccgggcgggcggagagcggggaccggcctgggctccgagttcagctccggccggggggaggggaggggctgcggctcacactgagcgcagcagcgagcaccggcctagggagacacagcccgggcggacccggcactggactgggccagtccctgggacccctgggcaccccgccctggggagggggccgcgccccagccttccgggacgaggtggcggcgcagagccggaaagcggggtggggggagggcggtggggaggtgttctgtccgaggctgcctggcggcgccgaagctcgcctccactttttgggcacttgccgagcgcggcgcccgaacttttcgctctatcaccatctccctcaagctgagatgcaacctccctaccctcgccccagagcccagagccaggagcccgatccctctgccgggctcaggcgcccgcgaacctgagactcggtacgcggctcagcctagctctttcccctcgcgcctcggggtctcctcgctccaaggttctcgaagtcttgcgcccgaaggactaaactaaactaaacttcggctgagtaatgggggaggaggggggtgcagatacacaatccctgccctatctcacgatcaggaaaggagagggctcataaggacgccccaattcgcgagcgcccttgagtctggccggtgccacggtcggttcgcgcgaagtgggcaccggtctcccgagccgacttcgtgtactccggacctgtactgcgctccggtagaggacccttccctttctcttagcttcttaaatcttttcttttaaggtgtaaatgtgtgtgtgtgccgatgaatgtttctgtttttcatctgctgactttatttttttttattggtataacttctcccttctctctccctgctcgtccgttttccaccttttctcttacttatcttctgccctcgctcactccctaacacacacattcacaccctctccgcctcgctctcccgttctcgccaggtcctgatgggtacagtagattttgataaaaagacaaacgaagctatcagtggggctgatgttgaagaatgaagataataatgtttccataggtggtgcttcaaatgccattatttctcactgaatatttaaagagatccctcggcaaagatggatctgcgcactcctggggtgtgagcggctcgcatctcccagaccccggggaacgtgtacgggagcacgtgtaaatcccgcacccgcctccatctcacaaacaggggtcctcgcacacccaccaatccacactgtcccaggtacacgaacgcgtttgtgtgcacttgcacactcctttcccggaatgtgcccgtgtttggagccgcccgcatccttctctaacaatcttggcttcttgaaagtccgggcctcccgggcaccgcatgcagccaggaacctagcggagctggcggtcccgcaaagcttttctcctggtgcgcgccgcgtgcgcagcaggaagctcccgggaataacttcacacgtgtttgttacttggtctacgtgtgatttaagattaagtgtaggaaaaaaaaaaattccccccccccaaaaaaacaccacaacaaacaaccaaacgaaatgaacatgcgaaaagcagctacgcgatgcttaattaaaaatgggcgagcagagacgaataaattgggggatccaaactttaataacttttttcgtttttttgcgtcctcgacggcgtagaggcgggcgtttcgatctgcgcttggcgtcccccgctaatttcttgtttgttttctttccctcggtggccccgggaaaggagatgggaggtggaggagggggggatgggaggagggggatggtcggaatggcggggagaggagaattggtctttattgttgatggcaatacatcaattacgggccattgtctcggtcccaagttccgtggttcgctggtgcgggcgctgcagtgtcagggcgctggcgaggctccgcgtgccgcgatgcaaagaaatacatcaataaaaacagaagcagagtgggggttattctttatttttgtgcgggtcgaaagtaggcggcgagcgggagtgaggtgccactcctggagtccctccacgcgccctgctcaccccagagacctgcgcgctctacgtgcggtagcaccctcctaactctgggtggaggtggggtgggggggggactactggggaatccgggtagcgaagtttggacttgcaggttccgccagagggcctggtacttaggggctgatccgcaatagcgaatggggatagcccgaagtgaggcggcagctgcgccggggtcgcgacctcactggcggagaggaaccgcacccgggggaggcgcttggcgagttttgcaaactcccatcttgggggattgatgctcgttcctactaatacgtccccttagttttcccaagtcctagagtatccttggtgggttggcggggccgaaggtctgtgccccaaggccgcaggccacaactggggtcccagccatgaggtttccaggcttctttgctcaacctctggtcctacgaagggcaacgtggggggcgtcaaggctcgcgcattctgaggcctggttcttctaccctcaaaagtatcttattctggcccgatctgacctcgggtctgggatggggcctgggatgaggccttgcctgggccccagaccctcttttccgttccgttgacggtgcttggccaccctaccccagaaatggaggacgtctccaagtgctctccctgcacccctgtttcctcagtccccagctcaaggccccgctggcccccggccgaggctagaagggcagtagtggaggtttcgttttcttaagcaaggcaaaaagcggcgaaaaggcggcagcggcgccccgggggcgggggacgaggcacatcgatggtatcaacattattgatggggaaaagttcggggcaggagcccggagatcaggttcccagtccctcccctgcccggaggtgggtgggacagagcgggtggccagctgtgtgtgtgtgtgcatgtgtgtgtgttggtgggggggtgttcctgaatgcacccgcattaaccacctggggtttatttgtattttccttgtctgaaagtttgcgggggcagagggaaaaggaagggttgaggtggcttgtaccctctagcccttagggctcttaactagggttggggttggggttggagttagggttgggggaggggtgcggggagaacaggaaaaaaatgacagaaaaaaagatcaagtttggaaagctctccgtagattcggttatttttccaaaccccgggaccttgcttccccgggagtttcagaaactcctttcctactgaactgctgtcactcaatcctaacctctcttagaacactggagcggcttcgaacccgaagtatggagcaggcttgggtgggtacctcgcccctggccaagtctacactgtgcgccctcggcgcactcgctctccagcagttcaataccagcagcaaaacgcaggtttttgggggaactcccgccgcccgccaccaagggctatctccagacgggcgccgggtgcagcgccgtgaccgggcgccctggcgccggctcgggcgcgaaattcagcggtggcaagcggagggtgggcttggtaaccacccgcgcgcgcccgagccaagagtcgcgtactgtctgcccgcggcaaagttcgtctttctccgcttggagggctgttcctacaccggtattaagaaaccgacttcgctagcgactgcaagtgcttgcgattttgactttccgtccacagttgagcgtcttgcacttaaattcactgcgccccgcatgcaacagtgcctcgccaaggggcctctgggtatgaaattcctcccagaggaaatgtcccgacgtagagaaagtggaaaaggctaatcgaatttataaaaagaaaatctttcctccttttggttaaatccctttgcggttttctgagcgcgaaaaaaaaattatcaccatgtaatagagtgtagctaattcttagaacctagtagtaggatgttcaggaagcccagcaggacgcacagattcccatctgccgctccagtaggaccttagttaggatccagcgaatagtggttagttcctgtgtcttctgttggaaataggtgtgagatctgtggagtcctgttctcggagcgtgtgcatttggaagagactggtagtctgccaggaaactcactttcattcataaatgagatcccctcaaacgacctccttatggcattcctggatgaactaacttagcacatctcttgcaacaaagttttatttaaaaaaaatttacgctgccacttttt

SEQ ID NO: 94 - DNA, 5474 bp, Homo sapiens
ttttcaggagcaaagcccccattctcccttcatccacttcttacagtctaattgactccctcctccttcttctttgaacaacgagaagactctccagttgtccaacttccacccctccagctgcaacatcgtcatcctttagctcagcgaacgctgggcgaacgctccctccccgcccctcctgcccctcctccctactccaagccctcggcttctcatccgctgaacgatgtcctacttcgctcgtccttgctctcgccgctgctgccggagccgaagcagagaaggcagcgggtcccgtgaccgtcccgagagccccgcgctcccgaccagggggcgggggcggccccggggagggcggggcaggggcggggggaagaaagggggttttgtgctgcgccgggagggccggcgccctcttccgaatgtcctgcggccccagcctctcctcacgctcgcgcagtctccgccgcagtctcagctgcagctgcaggactgagccgtgcacccggaggagacccccggaggaggcgacaaacttcgcagtgccgcgacccaaccccagccctgggtaggtgagtgcctccgcagccccgccgcccgccgtggggtcggggacagggagaagggagtgcctgcctggtctgcgccccccgcctgtcagcccttgcctcgaggctctggggcacccaactcgtcgactcctgacaccgcagcggggtaggctgctggacagccccgagcgcctgcagctgctgctgccatctctgatctacatgcttccagctctgccagtggcagcccccctgctgctcgcagtctgatcagcaacccctcgggtcctcgcatcttccctgctgcgcgctcctgtcccagcgcggccgccgagatttccgaccttgtcccaggcagggcggtagcgttccggatcagtccttgcctccgtgcccccacgcacaggcagctccaggagcagcggccagcaacccttctggggacaccttactgtagtccggcaggacagccgatcagagccgctctagggggtggtcggagtgcccatttcggctggaatcccaactcggctccttgctctgtgaccttggggtagttagttgacttctctgaacctcgggttcctcatcggtgtaatgagcaaaacataaacaatctttttctcacagttgaggttatccacaggaagcatttaggagaactggctcaggtagacatcagtaactattagctggaattcggttaccactattgcattccttcctagcactgccaggggcctgggaattccctctcccaggtatttccatatcctgtcaggtattcccccacctattggaatagctctgcctacatggttctaataggaccaagagattggcgtcaagtccaaggggcaccattattatctgcattacttagattgcagatgatgacagtaagatcagggaacagtgttacccttagacccacccacagtattaccagtgcctatgttgctatggctaactttgctatttataacagcatcatccacatttcagattctatacgagagggatctgcaactgctcggctggtgtcattgctaatagtattattagttttgcttccaataaaaaggactatttatatgatgattgcaattacttctacaggccctaaggccctgcttatattgtgtaggctcatgttgctcctcatgaagccgttaagggggtagcagagagcttccctcctgctatagccaaagctctggcttcttgtgcctccctgctgaccctgctgagtgctggagctcagttcctatgggacagaccttggctgccggctgatgggcattcagcatggaggtggtgggtgttgttagatgtgtgtacacacgtgcatgcctacacacacacacacacacacacacacagagagagagagagagagagagagagagagagacctggtcttaactctctgggttgggtctatgaagactgtgagggagatggacatcccgggtcagacagaaatgatgtgacaagaggaaagtttcctgaacagctcaagaaattcaaaacaagggcagagatacagacgctggagctgaaacagagagggggatgttgtggcgtcaagggaacggaccgcatgactgctgagctctcagggttagagttggagaggtgtggcctgctctggaggaagagaagaggaagtggagatgagtggtgtgtgggatagagaaggccacagaaccctggacccctgggttctgaggctggtaccggatgtggggaagtctgagtttcctcagggctcctagctgcaattgggaggctgaggcccgagactggagagacagagctctctcagaactgcacggggctaccctcttggcctcactggtcagtgactcccatttttggggtcaagggtaggttgccctccatgttccagagcagttaggtcctcattggccttaggccctcttccctcactctcccactgtcagacagaggtgaaagggtggtccagggtgttggccccaaaatgactccaactatatgggtctctgggctgcccgtcagagagatgggagccaggtctgtgagcagctccagctccagcccagagagaagggagggagggcaccagaagggggccaggcccaggccctggcacagctcagatgcatggcaaacagtcttcccagcacccggtgggcactggctgctcctgcccaatggtccccactccagccccagaacagtcctcccaagcaccccaatgcctggagctggtctccgcacctctaatctggccccatcaagaacctcctccctctgaaaggtcagagagaggccctaagcatctggttgtgccactcctctcccaactctgaccctagggaatgatatccagacacagtgggggagatgggggttatgatggcagggaggtgggaggatctgggaaaagtaggaaatttcccatcatctcaggcttaggaaagtgcagggacaggtatgaaggcagatgctgagagggcagggaagatgggggataaacagtgggcctggaatgggagtgcacagccagtggaataaacctgatctctaggaagtcctgcctgtagtctaaccttcaattctcttgctgcatcattggcctattccatggcatacctggatctccatgctcttctaagtttagcccaacccttaaagatgatgtagctctgagtcttctctcctgggagcacattgccctcgtgcctgacttggctttactagggatccagggtcagccctatccttctctgagcctcagtctccatttctcaatgtcacaacacatcctttttatgagagtgtaagtgcttcgagatctttggaggaatattctgggttctagagcttctgatagccaggggtaggaaggggtagcccctcccatttattctccccccccttagtccaccctccagcccaaggctttcacaaaaggtagggtgggggaagggaggggctggatagaggccagactggcagagacaggggctttgtcccaactgatttctgagccaagagttccggtgggaaagcccgtgccaggcagaaaggagaggctgggaagaagaagggaggaggcagccaaggaagggggatggagaatgttggggagtaaagagaggagtttccttacaaataatggtggggaggaagtagaggagagggtgggctcaggagaagctgtgcttacttgcgttcggtctaggaggccctcctggaaaaggccaggtgagtatggtgtccctggaggcagggacagtctagaaggcagaagaggctaaagagatactatgggagaaggctctgggttcgaggaaggagtctggcactgggaggagcagatggaattctgagtataagagcacccaccattcatgctgtaaactgagctcctactgtctgcctggtgctatgcatttgtacaggagttttaagatactaaacactctcaaagcacatggtcctatctcaactcatgagatagtaatgatgatgcttttttataagtaagaaaataaaggctcagagaaggtaaggagcttggcttaggtcacacagtgagttgactaggactcgggttttggttctgagaagaaatcacttgctgggaagatggggatcagaaacagagagtccatggtgatgtgaaggtaggggacttctggagccaggatatgtgtgaacccacatgggtgtggtagggagcaggggggaagtccatcctgaagtctaaccccatctttccttctcatgtccctctgtcagcctgcagcatggcccagctgttcctgcccctgctggcagccctggtcctggcccaggctcctgcagctttagcagatgttctggaaggagacagctcaggtaagcaaccccacttggggtcaccgtctctgtcttgttcagccctaaccaactgcttccaggcttaggggccccaggaagagagagaattggagggctgtggggaagcgtccaggctggaacacttggaaataagggctgagctgtgagcatctggtgggttggagaaatcatggggcaggactagggaagggcactgggtgcaaggctttgggcccctaaggtggaggataggggatcctgaagctaggaggtgggaaaggggtggaactgggagaaaccctgaaggaggccctaaagggagcagactgggaaactgggctggaggactcagggggcgaggttggaggaccctggggtgtggtctaggaattttggagaggaagtggaagactctgagagcagggcaaggaatcctgggggctgaattgactgggagactttgggggatgggtctggagaccgtcggggtggtcctggaggacctagaggtagggctgcaggaccctggcccctggcccctggcccctggtcctaggggggccggggaatcctggggccggaaggagggatcctggagcggggcttggaggccaccgggtgggactctgagggtcgacagcgttaagttccagccggctccacccgttcacagaggaccgcgcttttcgcgtgcgcatcgcgggcgacgcgccactgcagggcgtgctcggcggcgccctcaccatcccttgccacgtccactacctgcggccaccgccgagccgccgggctgtgctgggctctccgcgggtcaagtggactttcctgtcccggggccgggaggcagaggtgctggtggcgcggggagtgcgcgtcaaggtgaacgaggcctaccggttccgcgtggcactgcctgcgtacccagcgtcgctcaccgacgtctccctggcgctgagcgagctgcgccccaacgactcaggtatctatcgctgtgaggtccagcacggcatcgatgacagcagcgacgctgtggaggtcaaggtcaaa

SEQ ID NO: 95 - DNA, 5414 bp, Homo sapiens
tacacagtgaagcgcggtgtctcccgagcctaaatgaaatttcaatctaataattccttcctggcccagtcataatttgtttagagatgttgttctacttctttcaaagcgctattcgcactataattaaatgatactcaagcttttaactttgatttatttcatttcttgaagcttgagacagagctgtacaatgtcattttttttttgtttccttgaaaattactctggctgttgttgaggtagaaattaaacacctaagcacttacttgaaccgtccggcacaagccacattcattcacgtgaacactcccctttccctaccccatgtccaggtttcgctgagctcacacccggcaacactgctgctaggagttcccttcggctactatttattattttcctccacacaggggaagagaaagggaagcccgagaggatccagggaaagcagaagggggttaaggaccatggacagagcccgtcgcgcgctcgttgctgccgccttccccagcactctggcggctcctgaggacagcggtcccatcttgaaaccgctattccgcccggctgaggtcaggggtggacaggcggtcccctactctccaccgccgcttccgggagctgaccacccgagggttccccttttccactctccttcccactctgtttttgtcccagcgcgcgccagcgcctctcaggcctgccgcctgctctcgcacctgctcgccttccccaggcgcccagtgcctgcacctgctcccggtcaacccccgtccggattgggccacccgcgggttcctgcgtcggggtcccggggccttctcaccctcgcctgcaccctgctccttccgctctctagggaggtgacagcagcccccaacaccgcgggaagtatagagaaaatgggatccagaaggagaggaagtagtgtgtgtgtgtgtgtgtgtgtgtgtgtgtgtgtgtgacagagagagagagatagatagaaagagattatctccttttgcaactggaaccaagagtgtgtgtccatctctaggaaaagtggtctgcactgggactgggacagaagtgggagtgaagtgtcagctaaaaataggctccgcaccgagaggctgtggaaatgaagataagtgaggtttgtgccagcccccgagggtgtgtgtgtgtgtgtctgtgttgtggggtgtattcagcagcatatgcgctgtgtaatttctgaccttccctctccctgtcagttgccccttcttcctttgattgtggctaatgaagaataataaatccaggggcagggtttgccagtggatccttccaagactcaactcgaactgtactggatacagggaggaggaggaagagaaaaggggggcaagaggagcgtgtgtgtgtgcctgtgtgtatgtgtgtgtgtgttgtgggaggggtggggacagcggggagggggaggagtcgcatgcgcacagacgacccgagcctgctccgcggctgtccaatccgctgagagctgcgagaaatcgagtgagagaaagccctgcagcccctccgaccccatgtctctttggcaccaggcacccgccgggccgtggggggctcgtagccgaacgccgacctccgctcgtattgggctgggagttcagagccgcgcgcagaacccgggttggccgcaacgtctgtgttctcagcggtggccgggaacctgggatcagggtcacctgagctgacggggtgggggcgggccgagtggggttggaagcctggaacttagtggtaagcaggaggcgtaggaggtggcagccaggtaagaggcactcttacctacccaacgctggcttgggccgcaactttatttgggagtttctttttccggtgagacagagacccggcagaagaagcgggaggggctggaggctggtccttaggtaggcactgcccggcgactggagcgcggacctggccatttgggtggggttgagtgggggcgcgattgtgagtagcagccgcgggacgctgcgaaggggcggcggcaacagagcacgggcgggggcagaaaagaggcggcggagggcgcggtgggggagcgcgaggcgagtgctgagagagcagaaaggactcaagcctgaggggagtagagaggaagaaggggcaacgcgagaaaccgaacaggagccggcgtttcctggcaagggagggcggaggcgcgcgggagagagggagagagggagggcggggggcgcgggggtaggcgcggggagaggggagtataactcgccggccgcgaggagcgggggcagtttcgggtgccgaggtctgcagctagcggcaagcggagtcaggcatccgttcagactgacagcagaggcggcgaaggagcgcgtagccgagatcaggcgtacagagtccggaggcggcggcgggtgagctcaacttcgcacagcccttcccagctccagccccggctggcccggcacttctcggagggtcccggcagccgggaccagtgagtgcctctacggaccagcgccccggcgggcgggaagatgatgatgatgtccctgaacagcaagcaggcgtttagcatgccgcacggcggcagcctgcacgtggagcccaagtactcggcactgcacagcacctcgccgggctcctcggctcccatcgcgccctcggccagctcccccagcagctcgagcaacgctggtggtggcggcggcggcggcggcggcggcggcggcggcggaggccgaagcagcagctccagcagcagtggcagcagcggcggcgggggctcggaggctatgcggagagcctgtcttccaaccccaccggtgcgtatttctgcataatcaccgcttaaaggcacattttgacagccccctttatctgcttgatgtttttttcatgtctgcacagcaaatcaccccacacctccaaccaattttcccctctctctctcttaagtattcagcaggtcttgcctttcatattaatttttatgacctgggatgttgcctgtgcgcgtgttgtgttgtgtttcgttgtgtctacaggctcactttcctcctcctcctgcactctcggcttctttctgtggcttccctctttttctcttcacctctgttttcaggattattattattattattttaacgatctgggaatgttgtaggcgcggcgacggtgtcgagccctgggccggggcttccggagagagggcgtacaattccctgctgagcgtaatgtgtgccttctacttacaattgcagagcaatatattcggcgggctggatgagagtctgctggcccgcgccgaggctctggcagccgtggacatcgtctcccagagcaagagccaccaccaccatccaccccaccacagccccttcaaaccggacgccacctaccacactatgaataccatcccgtgcacgtcggccgcctcttcttcatcggtgcccatctcgcacccttccgcgttggcgggcacgcaccaccaccaccaccatcaccaccaccaccaccaccaaccgcaccaggcgctggagggcgagctgctggagcacctgagtcccgggctggccctgggcgctatggcgggccccgacggcgctgtggtgtccacgccggctcacgcgccgcacatggccaccatgaaccccatgcaccaagcagcgctcagcatggcccacgcgcacgggctgccgtcgcacatgggctgcatgagcgacgtggacgccgacccgcgggacctggaggcattcgccgagcgcttcaagcagcgacgcatcaagctgggggtgacccaggcagatgtgggctccgcgctggccaacctcaagatccccggcgtgggctcgcttagccagagcaccatctgcaggttcgagtccctcacactgtcccacaataatatgatcgcgctcaaacccatcctgcaggcatggctcgaggaggccgagaagtcccaccgcgagaagctcaccaagcctgaactcttcaatggcgcggagaagaagcgcaagcgcacgtccatcgctgcgccagagaagcgctcgctcgaagcctactttgccattcagcctcggccctcctctgaaaagatcgccgccatcgcggagaagctggacctgaagaaaaacgtggtgcgcgtctggttctgcaaccagaggcagaaacagaaaagaatgaaatattccgccggcatttagaagactcttggcctctccagagacgcccctttcctcgtccgctcttttctctcctctcttctgcctcttttcacttttggcgactagaaacaattccagtaaatgtgaatctcgacaaatcgaggactgaagagggagcgaacgagcgaacaactgagcccaagccggtgagaatgtgaaacagtttctcaaaggaaagaataacaaaagatggtatttgtctgttgtagcaaagttgtccctttgaaccccacctcggcttcttcagaggaagtgtggagatggctgtttgcaggaaggcagacgagacagtgtttaaaaagtccacaagaatgatcaagtaagatttgtttttattcttacagacatcacccgtgttcaagtttaaaagtacactttgcaactatttttcagaaatagaaattgattcaggactaaaactttaaactagagttgatgcttaatgtgatagagacatctctaaagtattttgaattttaaaaaaagatggcagattttctgcatttacactgtatattatatatatatttttattgtggttcttacccccttttccttctctgaagtgttaatgcttaagaaaagagttgcgcctgctgtgttcactgatcttgaaagctattattagattattgcagaacaaccctctgtaaattattaatttatctctctagcaacttaattttgtgcacattctaattaattaaacttcttccgtctaaaaaaagtgggggaaatgtatagctagtaacgttcaaaaaattttgtttgatgagtttaccgaatttttacagctttcctcctatactgtgttccttttgacccatttgtatattctcacttgaatgaagattgtttttttctttgtttttactggtagtgttctgatttgtgagtcgacactcagtaatggatgtcttaatcgtgtagacctgattcactgtctgaagtattgtttacttcgttacatatttaatggggattcccacattgtccccatgacacatgagcgctctcacttacccttacacacacacacacacacacacacacctctaacagaagggaagaagcagttggaagcatgaccgatgcaccattttctagttttaggtgcatttgccacttggtgtttgcccttcagattttagatttcaccaaggtatttcagtcttccagttt

SEQ ID NO: 96 - DNA, 3381 bp, Homo sapiens
ttcatcatttctggggagttctcttggggtccacagaaaatctcacctcatctcggtcccctcagcaccggtgatatcttggaactcggccccggttgttcgctaaggaggacttggcttgggggcgggagggtgctccagagttcctgggggtccaccgcactccccatccgaggctcctcgcgagcggagccgcttctccaggccgcccgcctccctgccgcaggagcaggcccgcgcccgccgcccgagagcctccggtagagggagacactccagcggctcctccgacttggcagcggccgggccgggagtgggtgggggtgatggggggtggggagaaaagggggagtggggagaggggaggtgtgtgaagggggggtccggggggcgggtccctgtgccgctgacgtcccgagcagtgctgggaagtataggctgtgttgtcacgccggtgtcagtctgatgaagattggcatcaggtaagctgtcattcatttccatgtcagagacgcttttgcaggcggcggcggcggcgcggcggcggctgctgctgcgggcggctgcctcagagcgcgtgtgttttattccagtccccaagccagagtattattcattgcgacagggcaaggaggagagagggagagagggaggcagcagggaggagagagagggaggcagcagggaggagggaggcagggagcagcgagggacggcgggagcgtgcagagagaagctggggaagcgccgggagagcgcggagcggagcagcgcgaggggcggcgaggccgggcacggaggctgcgagagccccgcgggccgcccgctcccctgcccggccagcgctcagccccgccgccgccgccgccgccgcctcgccgcgcttgggcccgtggccggccgcgcattgtcctcgggtgcaaggagccgggctgcggactcgaatcgccgcgggcccaaccccgtagcgggcgggcggggagctgtgcgccaggagcgccaggggacccgagaataggaacaggcacgccggcccgagcccgggtgcagaaggctcccggccgggcgctccgcggggagaggctgggaaccgcgggcaggctccaggttctctttctcccggcttcgggcgtccttggggccggcgcttactccgcgccaccagcctggagatgctttccagcggctggaggcgggagcggtcccaggctggggccaggtgaccggaggagtcgaggagccgcggccgcggcctggggaggcagggcgaatgagggtttgcagacggatcagtggagacaggggaacaccggcggggcccgggaagccaggatccgaggggctacacgcacggaccctcacccagggaggagcgagaatgtgtagggtcaccggctttccatgattgctggggatctttggtgaggcgggcgtaggggcccgcgcgaggcttgggaatcgggagcccttctggctcgagaactaggggatgagttcgtaaaagagggaactgaaagcgatcgagagcggagagctgagggggatggcgcaggatcccgaatctgccgctcaaagtttgcatttctttcgggttaggagacgggctttcctggctcccgatccccaggaagaaacgagcgaaatgggccgtcctttcccggggctcttcacgggggagccgggggtttccgcgcggtggggagactcgggttgggaattgaggggtaggggcttgtggatcggcctgaattagggctgggttttaggaccagtctagagttcggtttataggatccagactgtttacggaatcgggatcgaggggccgataagtagtttacacgccggccagagcagagggctggaggtcggagttgggggctggaggaacgggtggcgtttttaggattcagtaacaggatcacagctttttcttgtggtggaagctattggaatttggggagggtagcacgaggggtcctgcagctccgcgtgtgaaaaagcgtttaggtaggcgatgaaagtagttgatctgagccatggcaggcgagccccgaatttttgctgcttccccctgaaagtgtttctttaggaggagaggacttgggccacacaggacccggtcctaagagagcgattccgggaagcggacagatcgaagagaccttctgggcgaagcggcagggcagcctcgcggggctgggagtggatctgaggtcccgacccaggcggctcggagtgctccaggagccacctgggtctgcgggcgcagcgcggcggggcgggagcggtggcccgcaggggccgcggcctgcgatgaaggccggggggcagcgctagcagcgaggtgccacagtgggccgaggagtctgggctgtggcccagggtaggaccggctcaaactccagtgccctgattggagccgcttcctgtgcttacccgcgccggactgagaagcccacaaacccggcctttggtgcgcccgggggaggaggaagcttggagtgccctactgtcatctctcctgtccggagacttcaggatattagcttttcgggtttcaaatgctctataaccggtggcagccaaaagcttcgcattttaggcagtttagacgatcctccccgccccaaatctgagaatgatggtgttcaaacataacacggtgtattacccaaagcccctgcccttgccctcctgctctctctgccgttctggccttcggaggccccaggaccttggtggaagcggggagggagggtcccgtttctggtctcataatctcccactcgggctgcatccccatagcttgggcctagctgtggggtgtggggtgggaaggggtcccgaccagggaggtaggttattctctgggcatctagagaaagagtgtgtgtggggggtgggagcgggggggaggagggcattgaggggaggaagatttgagatttcctcctcttaatattaaacccaccatcccagccccctgcacccctggcagggagagccagacagcccttacaggacacggaagacgcacgacccacgacccacgagtggcctcggcggcccggggcgcggcgactgccgccgcggagctccaaggctgttgttttgtaggcttggaattcgtgagagctctctcccgctgagagcccgaggggggagggagccgtgaccccacggagactccctggtcaccgcctcccctttgtgagcgggaaaggcttgaaagccaagagaaagagaggctgctcagaataaggggcagggggttaccggagggagatgggcccgaccaccgctcacagtaaaatgcctcgtgcgaattgcactgaagtatacccaacttgagactggctgttttatgatcctctctgg

SEQ ID NO: 97 - DNA, 1266 bp, Homo sapiens
ccttccattagacctggtcgcgggaaaccacaaaggacagtcaagtgattttcaaggagaaagcctctggtccactttccctccgccgccagatctgcaggttcctgggaggagctcttctcaaatcctgaagattgctaggctcttggcgaacgactcatctcttgaagacctagagtggtggattacaggcattgaaaagcttttggtggctttggaagatgactctggtgtgagctcacctttccaggctgggggaccaggcagaggaaccccctttgttatcttctgaaagaagatcagtggggaagacggggtttgaagtgtggattaggagatcctacgtctttgcctaccacctctgcctaaatcataaaaagatcgaggagtgcaatgaacttcaggaatcatgcaccgtttccctgaagcctgtccaggaacctaacttctggacccagaaacttctgcaaagacagacccactgagccaggcagtctgcaccagcacctctgcttctaagattctgtttcgtcttcttctattgagagattgacctcttgagtgatttgtgtgctttccggcaaatgatggagacgcgtaaaccggcggaacggctggccttgccatactcgctgcgcaccgcgcccctgggcgttccggggaccctgcccggactcccgcggagggaccccctcagggtcgccctgcgtctggacgccgcgtgctgggagtgggcgcgcagcggctgcgcacggggatggcagtacttgcccgtgccgctggacagcgccttcgagcccgccttcctccgcaagcgcaacgagcgcgagcggcagcgggtgcgctgcgtgaacgagggctatgcgcgcctccgagaccacctgccccgggagctggcagacaagcgcctcagcaaagtggagacgctccgcgctgccatcgactacatcaagcacctgcaggagctgctggagcgccaggcctgggggctcgagggcgcggccggcgccgtcccccagcgcagggcggaatgcaacagcgacggggagtccaaggcctcttcggcgccttcgcccagcagcgagcccgaggaggggggcagctagcgagcgcccgaactggccaggacccccgcgcccgccgcacagcgcgcagccgggcgctcaacctaaggtcctcttcgaaggtggtttgcattcttaatctggcatcttctccaggcctaaatcttaagaaaaagaaatgggtgctggggtttgggggatg

SEQ ID NO: 98 - DNA, 2968 bp, Homo sapiens
ttgcagccccgccctcccaggcttgcaaaggtagacggagaattatattggaatttaaatcggaagctctcaaggcatctcaaaaatactttctctattttttttttcctgtagatattggagaggttggcaaacgggtcttcctgaagacagaagaatgtatgatttaatgttttctttagatttctgtatgagtggatgcacagtgctccgtattgtgtggtggggcggggtgtgtcttcttattgatgaaatacactgcgcaggtcaactcggtaaattgaaatgagaagagccgactgcgggggtggagggggtgtggtattagggtgccggcgcttgtggaggggggcgcgaatgtgaacgtgtgaaagcgagaggcgtgccaggagagcgcgggaaagcttactggtgaggcaagtgtgcgtctatttccatggcgccctggctcgcggcagcccctggctgggcgaggggtgtgatgtgggagtggggtgggagggggcagcaggcggggcctgccacgtcacttggagagtgtgtgttgggaaggaagggcagagcggagagccgagccgctgcagctgcggcggcggcagcgaagccttgagccgtggggaggtgggtccccgcgctcgggcgccggggcagccccgggccctctgcgaggcctgcggcgcggctcctagggaggaggtggcggctgtggcggccggaaccgcgaccttggccggacccagccccgcggtggacgcagggcggaggccgagccccgccaggagtctttgccgagccggagggaggcgcatctggcgcttcggtaccagcggcagccgggggtccggagcggctggaggagcgcagtgggaactgggaagagctagcccggctggagggcggacctctgcgtccgggagccgggtctcaggcaccgctgggggcgaagccacgcgtcttttcgggcagccaatttcacacgcgcctgtgtgcggttccgggcatcccagtaagctctagcacccgggcgcgggtaacgggaagcgcagaaccaaatccccagcgcccaggtcacctccccagacccagccttgcagggaccagggctttagggctcacggacccaacggccaggtcagaccgcgaaccgggaggagcgcgggccccaccctaaagagggcgcagccgggagctggggagcgggtgccgcgctccagagattgtgtcgtgggcgccgtcctagtggcggggagcgcacctccgagggggcatgagatcggagaaatcccttacgctggcggcgccgggggaggtccgtgggccggagggagagcaacaggatgcgggagacttcccggaggccggcgggggcgggggctgctgtagtagcgagcggctggtgatcaatatctccgggctgcgctttgagacacaattgcgcaccctgtcgctgtttccggacacgctgctcggagaccctggccggcgagtccgcttcttcgaccccctgaggaacgagtacttcttcgaccgcaaccggcccagcttcgacgccatcctctactactaccagtctgggggccgcctgcggaggccggtcaacgtgcccctggacattttcctggaggagatccgcttctaccagctgggggacgaggccctggcggccttccgggaggacgagggctgcctgcccgaaggtggcgaggacgagaagccgctgccctcccagcccttccagcgccaggtgtggctgctctttgagtacccagagagctctgggccggccaggggcatcgccatcgtctccgtgttggtcattctcatctccatagtcatcttttgcctggagaccttaccccagttccgtgtagatggtcgaggtggaaacaatggtggtgtgagtcgagtctccccagtttccagggggagtcaggaggaagaggaggatgaagacgattcctacacatttcatcatggcatcacccctggggaaatggggaccgggggctcctcctcactcagtactcttgggggctccttctttacagaccccttctttctggtggagacgctgtgcattgtctggttcacttttgagctcctggtgcgcttctccgcctgccctagcaagccggccttcttccggaacatcatgaacatcattgacttggtggctatcttcccctacttcatcaccctgggcactgagctggtgcagcagcaggagcagcaaccagccagtggaggaggcggccagaatgggcagcaggccatgtccctggccatcctccgagtcatccgcctggtccgggtgttccgcatcttcaagctctcccgccactccaaggggctgcagatcctgggcaagaccttgcaggcctccatgagggagctggggctgctcatcttcttcctcttcatcggggtcatcctcttctccagtgccgtctacttcgcagaggctgacgatgacgattcgctttttcccagcatcccggatgccttctggtgggcagtggttacaatgaccacggtaggttacggggacatgtaccccatgactgtggggggaaagatcgtgggctcgctgtgtgccatcgctggggtcctcaccattgccctgcctgtgcccgtcatcgtctccaacttcaactacttctaccaccgggagacggagcaggaggagcaaggccagtatacccacgtcacttgtgggcagcctgcgccggacctgagggcaactgacaacggacttggcaagcctgacttccccgaggctaaccgggaacggagacccagctaccttcctacaccacatcgggcctatgcagagaaaagaatgctcacggaggtctgacccatgcaggcagggcctgcaggaggggagcactgagctaacagtctcttaggcttccttctcatttccactactcactctagcttcagttgacttcttgactctctcccc

SEQ ID NO: 99 - DNA, 4899 bp, Homo sapiens
gaggcagccctgggaggcactggtctgaagggctagacccttggtaagaacctctggaagaacgcctgtcttccggaaccagacagggaggcgtgcaacggggtggggtggggggtactcccagctgcaggcacctctactcctaactatctgcaagaggacacacacacacacacaccctgggtgtcagagaagtcctcccttccctgtacctcctccttcccaagcccctgaaatccagaagtgcctgtttccaggcgccagccctgctgagtggatgtggggatggaggtcactggaggcccagggctggtttgttgtttttgtttcctgttaggagtactgtgaaggctttgcagggctagttttgggaaatcttaggtttggcttcatctgactcctctactggagcgaggcgtgcagccaagcctgcttagacctccccaggccacgggcccgcggcagcccagctcagcacctgcaggcggggggaccgcgcgcttggctcgaagaccccgccctgggcaggggacagaaaatatgaaatcagatggggagttatgaataactgtcctcccctcccctctgccgtgtttcctgaatccgtacgctatgcaggaggggggcgggggcgggggcgcggggaggaggagggggccgtttgccattaatctgggaacaagcggctaacctcggtgactggtattttcgcttttcttttctcacttttctcagtgtggggaaagcagtcaagcccgcgcggagcgattgtgaggggctctgctggaatttggcagcgcggaggcttggagagaagccccatgctggctcccattcagccggcccgttttcctcgagctttggaagtttcactcagccgtgcactcaatggcttcacaaagctgattacaagcttcagcgcattcctgaaggagccaaaagcgacgcaggtgcaaacgagccgagggagccccttatcccggtgacagaatgggacaagctgggaaaggcttagaccacacaagtccaaggctcaccaggccgcagaaagcctgccttgggaaccgggggtcattatccgccctattcagcggggcccggggaccctgggggccgagcgaggccagcccgggcgggagcaccgccgccagcgcgcgccggcgacaggggggaaccccgctgggcgctgcggccaggccggggctcagggcgctgggctgtgcgtttgcacaaatctgtgttccggcgggccggtgtcaaccctagtggggacacgggagaaagagcacgccagtaggccacgcggcgcgtgggcagtgcgcaacttctgtcggcgtccaggctgtacggccacgtttcagccggtgcccccaggccatggacacccagctccagggtcgctctgcgccctttctccccagcccacctttcccattggtcccctctcctgtccgagtggcagcgcggccccgcagggagcgaccccgggagggacagtgaccgcacgcgtggagtggggacggcggggcacaggacggtagtctaagagtgttcgcatggccgaggacgctttcgctgcgagtttggggtcgaggggcagttccctcccggagtcattaacttcgtctcctcgtgctcagggaccagcaacttggtgctgcgggcgccagaaagaaaagggtagcgcctcgcggagcgcgcgggggaagagggactgcggcagcgggacgcgagggcgggaggggcgcagcagcggggagccggagcgcagcggaggggagcgctggggggcggggagcgctcgggcagccaccctgtccccgtctggagccccgcgctgcgtctaggagggcgcaacacgcagtccccgcgggggccagagctcggagccccctagtgcatgccccctctcctcgcgccctgccgaggcctcggccctgcctgccgtgccctgggcctggttgtgcccgggggtccccgcgggcagggcgcggggcaggcagggcgcgcgcgccgacgctcttttgtctgataactaatttgagttaatgcgatctttatgtaaagctaacagcggataattgtctattttctcgccaacagtctccatcacaatcacttatctggaaacctgcggttggattaatcgttatattcccgagatgagcgtcgctgcaatccgcagcgaacgcgggtagggacctggagacgcctcccgggcgctggcggggccggtgtgggccagctccgctaccgcctgagacgcggtgtgcccaggtgttggtgcccgcgggagagggctgtggggcgccttccccaggatttgcttgacttgcttttcgctcgagtgggtggtgcgcgggtgcgggaggcggagggaggaggaggggtgaggggaggagggcctgtggggctgcggacccggagcagcctgggtggagcgcggcctcgggaggccctgggtgcatcgcggcggggcctgggggggcccaggcgccggaggagccgtcggtgccggaatgcagcgtgtttcacttgggagaaacgttgccctcggtcccttgcctccctcctgttgtctcggtttttctggcttcgtccttcgtcccacccacccgttcccacctcaggtccctcccccaggatcccctccttaaggatccaggtctctgaaaattattggcaacagttacacgccccacgattgaaatccacaagagaagagtcccagttctgcaggccgctccagggctaggggtagagatggtggcaggtggtgcgtcaactctctagggaagaggaacttgcattacaaagacttgtctttctgagctgaagtcaaaacgggggcgtcaagcgcgctccgtttggcggcggtggaggggccgcgcgcccgcgctgtcccagccggagctgccctggctggtgattggaggtttaacgtccggaattcaggcgcttctgcagctcagatttgccggccaaggggcctcagttgcaacttttcaaaatggtgtttctggaaaataacaaattcagactcaactggtgacagcttttggctatagagaatgaaactgcttccctttggcggtggaactcttaaacttcgaagagtgaaagaatacaatgaaataaaatgccataagatcactggatttttcagaaaaaggaagaccccaaattactcccaaaatgaggctttgtaaattcttgttaaaaatctttaaatctcgaatttccccctacaacatctgatgagtgctttaagagcaaacgagcaaatcccacctcgagaatcaacaaacccaagctctggccaaggctctccccgcgttttcttctcgtgacctggggaatgtcccgccccatcgctcacctggctcttgtcatctcgctcatcttgaagtgacccgtggacaatgctgctccctgccgcagctccgccggtcactcaggcagaacggggtatgcaaatggcctggagaggccgaggccgaacactgcgcccattgtcccgggcgctcaaagcgccgagcagctgcgcagactttctgggctcggaccctcggccgccacgctaatgtcatcattgcaaaacatcagattttgagaactatgcagtctgaattcattagcagccttctcggcgaaggttcttttccccgtgacaagtttcaaagtccagaccccctccaacgttggtaggggtaggaagaggaggaggggcccagggaggggggtctttcgcaagtggggcctcgggggatccctgctctccttctggtgcattccgcaactttaaaagccttccttggggttctgagcgcggatgtctgacttgaatgacctgagatttggacatattcccgttttctcccaagagttgctttaatgttgcgagtttaaattcactgaaagaggcccagataccaagtcgagtgaatttcatagacatcatcttctgttttaatgatcgcggcagtgatcgtttctgaagtcataccctttagggtgaactcacaacaatgcatgtcagagaggctgcgcgccctaaagcaaaacagttaaaacatcaggccacgctcctcaaactgcaggattgcaagcagctcacaaacttccagtccccaccccaaggtgaggagagtctcagtgacttgaggacctgcttgcactcccgctttccacgcccggtcccctacccgcacctgcccaagtggtctcgttcctctagaatctgaatggtggggaggggtgctcctgatgccccgggtgcagggcgggcaccagcgagcgagacccaagggcgctagaacccaggagcgcaaaccaccgttagggcctgagggacgcagaggcgggtggagtagtgtttctcagattcgtgggaggccgcaggcgccagcagaggcaattttcctcccctgagcaatgctagttctcctcaggcccaggatctccactaacgtgtccttccacctcctcacaggactccaagctgctgtcccctctctgccgcatctgaggctgggaaaacttcctaggagaaggcaagagaaagccaccagaccagagccgaggactaaactttaaggtcgaagacggcagaggggcaggttctcccctgcacaccccaaggcctctcctgcacccgcgaggccttccttgagcgcccaggcccccgaaatgcctgccctccttctgacaaaaggagggggtaggatgtgaaggggtagtgcaaccaacaatgtttttgtaaacacaacaacagggaaatacatggaggaaatctatttattgggtgtgtcatttttggtggggggcgggattgaaagtggcgccatctagacccaggaaaaagctacggggtgggcggggtaagcgggggactcttgg

SEQ ID NO: 100 - DNA, 2346 bp, Homo sapiens
actcaccttgtgaacactcggacatggtgaattggcattggcattgctgttgaaggacaactcagccgtgttcttagccatggccatttaggcctgttctgatgcagggttctgatccaaggtaccagtgtggtccctcagggaagtactggggatcgtcacttatgcctgttctggacatggtcaccgagaactgtcctgtaggcattcacttaggaatcattcgaagtggaattgctcctggatacgttctccttgtactctgtttcctcctcctagtgtctctgtgtgaagaagccctcctcactcagccctcggcgaccctctggtaccctggacagctccccggggagcagtctaccgctaggcggcggctgctaagagaggaaccctcctgacgcggagtctgccgctccggggctcgctctccggcaggcccggggagaggtggggtgacaatgggttggggtgcgcgcgtgcctcataggtgcgagacagagcgagccgccggggtgtgagtcagcgcgctgggggctaagaagctgggtgaatagtcacggaatctcactcacgctcggctcctccacccatcccgtctacagcgcgtgtcccagtccagggcgtgcgtgcgctcggtgtccgattccgggctgtgtgtgtccatttggcgagatgtcgagagcggggggagtgtccttgtcggtgtatctgggcccaggttaggggacttctcctccccacccccgcgtgggtgtgggggtgtgtccgggctagggcgcgtgtgcttctgtgcctgtgcgtgcgtgtgcgggtcagggtggtgggaccgcgcatcagggcagggtgcctgcgtctgcgtctgggtctgtctggtctgcatgtcggcgcgatctcgacctggattcgtgtccctggatgtcgagaggccagcgtggtgggggtgtccagcctcccggaggagtactatgccttgacaccttcgtttcaccgccccaaagctggcctggggctccgtagggagtggcctgcatggggagggcccgcgtgctgtgtttctgggaggggtaagagagtgggggcgcagggggcgggccaggtccctgggcgcggcgcgggctcgggggacccgcgcggctgacgtcaggccactccttaaatagagccggcagcgcgctccgctcggcatttcccgaagagccagatcgcggccggcgccagcgccaccgtccggtccacccgccagcccgcacagccgcgccgccgccgagcgtttcgtgagcggcgctccgaggatcaggaatggggcttcgggcgctgggcgcgctccgaacccggcgcacgtaagagcctgggagcgcccgagccgcccggctgcccggagccccatcgcctaggaccgggagatgctggaaatgcaaccgcctgttccccgaggagccgctgcccccgggaccccctggcactgtgcgcaccctggtcagcagcccccggagaagacggcgcccccaacgcccgacccgcgtggccgtggcagcgccacgcgagccctctaggcgaccgcagggccacagcagctcagccgccggtgccccctcggaaaccatgacccccggcgcgggcccatggagccatggcctatagggtcctgggccgcgcggggccacctcagccgcggagggcgcgcaggctgctcttcgccttcacgctctcgctctcctgcacttacctgtgttacagcttcctgtgctgctgcgacgacctgggtcggagccgcctcctcggcgcgcctcgctgcctccgcggccccagcgcgggcggccagaaacttctccagaagtcccgcccctgtgatccctccgggccgacgcccagcgagcccagcgctcccagcgcgcccgccgccgccgtgcccgcccctcgcctctccggttccaaccactccggctcacccaagctgggtaccaagcggttgccccaagccctcattgtgggcgtgaagaaggggggcacccgggccgtgctggagtttatccgagtacacccggacgtgcgggccttgggcacggaaccccacttctttgacaggaactacggccgcgggctggattggtacaggtaaggaccaggagctccgctccgtgcgccgggtctctgatcgcttccattgggagagccatccgtctcttgtgttttctctttcttttaacccaactcattgtatgggttcaggctgacacacagggccatggggggctatagcagaatttacccagaacttcccagtgataatctagacgggcagtttctggaactgcaaagggcgttccctcgtcactggagtcgttg

SEQ ID NO: 101 - DNA, 2695 bp, Homo sapiens
gttcccttccacgggggtcccgaggtgctttgaggagggagaaagagggcgtcccctctggggagcccactctccgggcttctactgacctggtctccgcctcaccggcctcttgcggccgctgcagaagcgcactttgctgaacaccccgaggacgtgcctctcgcacagggagcgcccgtctttgctggggctggagcggcgcttggaggccgacactcggtcgctgttggactccctcgcctgccgcttctgccggatcaaggagctggctatcgccgcagccatagctgctcagcgagggcctcaggccccagcctctactgcgccctccggcttgcgctccgccggggcgagggcaggacctgggcggccagggaaagggcagtcgcggggaggcagtgctaaaatttgaggaggctgcagtatcgaaaacccggcgctcacaaggttagtcaaagtctgggcagtggcgacaaaatgtgtgaaaatccagatgtaaacttccccaacctctggcggccggggggcggggcggggcggtcccaggccctcttgcgaagtagacgtttgcaccccaaacttgcaccccaaggcgatcggcgtccaaggggcagtggggagtttagtcacactgcgttcggggtaccaagtggaaggggaagaacgatgcccaaaataacaagacgtgcctctgttggagaggcgcaagcgttgtaaggtgtccaaagtatacctacacatacatacatagaaaacccgtttacaaagcagagtctggacccaggcgggtagcgcgcccccggtagaaaatactaaaaagtgaataaaacgttcctttagaaaacaagccaccaaccgcacgagagaaggagaggaaggcagcaatttaactccctgcggcccgcggttctgaagattaggaggtccgtcccagcagggtgaggtctacagaatgcatcgcgccggctgcggctttccaggggccggccacccgagttctggaattccgagaggcgcgaagtgggagcggttacccggagtctgggtaggggcgcggggcgggggcagctgtttccagctgcggtgagagcaactcccggccagcagcactgcaaagagagcgggaggcgagggaggggggagggcgcgagggagggagggagatcctcgagggccaagcacccctcggggagaaaccagcgagaggcgatctgcggggtcccaagagtgggcgctctttctctttccgcttgctttccggcacgagacgggcacagttggtgattatttagggaatcctaaatctggaatgactcagtagtttaaataagccccctcaaaaggcagcgatgccgaaggtgtcctctccagctcggcgcccacacgcctttaactggagctccccgccatggtccacccggggccgccgcaccgagctggtctccgcacaggctcagagggagcgagggaagggagggaaggaaggggcgccctggcgggctcgggatcaggtcatcgccgcgctgctgcccgtgccccctaggctcgcgcgccccggcagtcagcagctcacaggcagcagatcagatggggattacccgccggacgcaaggccgatcactcagtcccgcgccgcccatcccggccgaggaaggaagtgacccgcgcgctgcgaatacccgcgcgtccgctcgggtggggcgggggctggctgcaggcgatgttggctcgcggcggctgaggctcctggccggagctgcccaccatggtctggcgccaggggcgcaggcggggcccctaggcctcctggggctacctcgcgaggcagccgagggcgcaacccgggcgcttggggccggaggcggaatcaggggccggggccaggaggcaggtgcaggcggctgccaactcgcccaacttgctgcgcgggtggccgctcagagccgcgggcttgcggggcgccccccgccgccgcgccgccgcctccccaggcccgggagggggcgctcagggtggagtcccattcatgggctgaggctctgggcgcgcggagccgccgccgcccctccggctggctcagctggagtgctagctccgcaggaaactcggggcccgggcgagagccaccgagatggcaggtgggacgcagagcccgcggcagccagagttcctcccgcacggcccgccgacccacggaagagcgaaagagcgcccaggtggggccgagctgggggccgggcccctggagcgctgggaagcacagcgcgctctagtcaggttccctttcctggagccctccgcttccagactcccttctttcctccctccctcccgccacccctctccctcctctctgtgtcttctgtctctccccttttctcctctctacgcaatcctacgtgattgaggtttggatgagaaattctcagaggcagagcgagggaactgcagcttgggtctgctccgtccggtccctcccacaagagaaacacaaccacagtgggagttaaaggaccctaggtgcgcaaagaagaggtgggatgggggagctgagaaaatgcagtccacactctctccaataagcttgagcacgtagaattctctgtttagttaggaagaaagtgaacactggagaaagtaaaaatgacctcttggaccttatcgtgggccccacctatggctcattttggaa

SEQ ID NO: 102 - DNA, 2837 bp, Homo sapiens
aataatgttcctgctgccatcccagctggcttttcgtttgtccaaatcgctctctagtcgttgtcttttcacttcataaacgccactgagaattgtgcccaccacagccccaccccactcccaatcccactcccaatccccaggttcagattatggtatttggtatagttcttgaactccacatagcccgctccggaagcgcactgcaaaacgcaccccagatgctttcccaaatctattccttcattctctcatttcagaaaatcgctctatttattcaccctgagaaagtggactcaacccccgctttcgcgctggagaaggaaacacaatcattctctctttctcttcccctccctccccaacaccttccctccctcctcccttgcccctcctaatctccaaccctaagagcagcactcagacatgcaaactttctaattccttccaccctcccctgtcccaaacactgggcaattagactggagataacagacgcagggaaatctgggtttccatctgtcaaggagtcccctttccccacacccgctctcatgcccccgccccctcgaggggctctgggggcacctttatcgcttctcccctgccctctagaaaaccacccgtaactggaggctcccgcgcggcagctgcaccaacagcaggtgatccaggtggtgcgaggcgatgccttgttatggcatcctgccctctgcagggacaccactgagggccacatgaaatgacccccagcagtcctgcaaaagcaaaggggtggaggagggtctatgcaccctgccctgtgccccagtctctcccaccctgtcctccgcaaccgaggagaccctggcatctgggagcgggctgggccgagaaggcaagatcaataaagttcagcaagtagacagctgggagctgggtgctactgagttctagaaagggggagtagagacggaaatatccgaatcggtgaaatgtaaatcgtgtctactgcataagggaatcgtgcagggagagccgctctctataaggagttaaccagcgcctgccccggacacccccgcggggcctccagcggggctcaaactggcggcggctcagcagggggcgaggggtgctgggaaacgccggggctgcgaacttacggaagaaaatgtactcggtgtagctgctccagatcttgtcgtcgcggtactggttgacgaaggcggcggtgcccgaggagttacacgccaccatgtggaagccggcctcggacaggcgatcaaaggcctgctccaagtaggtgaacttgaggtagaagcgggacgtgtacttctccggctgccggtcggggtcgcggctctcgttgagcgtgtccccgaagacctccttggccagcgcgatgcgcccgcacaccatgatgcgcgccacacgccggaatttggcgtcggcctggttgtcgcgcacggtggtgtaggagccccggtagcccagcgtgaggaagcccgagcgcttgtcctgcgcgccgccgccgccgccaccaccgtgcgctcccgggcccgagggcacggcggccgccgccccgcgcagcagcagcgcgtcgctgctaccctgcgagacgttgtcctccaggtcgctctggcagccctcgtcgttgagagagttctgcttggtgaccttgggcgacagcagcttgaccaagtcggtgagctggaaatactcggcctcgcgcagcagccgctccttctcggggaagtgctccggcagcgcgagttgcttgtcccgcagataatccagcacgtacctgaaaaggaagccgtcccggtcgatgaagaagcgcgcccggctgtccctgggcagctcgccccggcgccgggcgccgccacggggactagagggcgagaacatgctggccaaagtactgtccgggacgctgagcagcgtcgagtgcttggtcacataaacctggccgcctacgttcagctccactacttcagggaagggcgagggtgcgcagggccccggggcggcggcggccgacgcgccgggcgagctggacgaggaaaccatctcgctaatgggcaggatggtgctgccgccgctgcccgtgtccttcagagccatagtccccccgccgccggcccagtgacccgagagagctgcactttctcgttcccggagcccgcgccccagccctccgcgtgctcctggcgctctgcgccctcggactgggcggcgcgttcctccgaccggggcggccccgctcagggttcggggcagcggcggcgtcggcggcgcccgagctccatcggaggagagacgcgcgagagaggagctccgccggtgcggcggcggcaatggagaggcaagaaggagctgctgctcctttggggcgagggcggggaagtgtgagagagacttgcgagaggctctagggctgctgctcggctctccgtggggagggcgggaggaggggccgaagcaaagaaaactcgccgccccggcagcttgcaagcgtgccgcgaggccggagcgccgacggtggcgctaactactcctggcagcacgggaggaggcactgacgtcaagccccggactgggacccgagcctcagctgtcatttaaagagataggctgcagccgcggctcccgggctccgacgctagggggcggggccgagagccgagcctgcaagaggtgcagctccccgacccgcggggacaggcaggcagggagctcctaaagcaaaagcgagaaaaagggacagcgctctcattccttcatttcactccctccttgggtctgtgccaccccctttcctccctcctttccttcttctcttccttccttccttg

SEQ ID NO: 103 - DNA, 7618 bp, Homo sapiens
agaggtccagaaggttctctcacctggcacttagcaaggtcaaggattcaccctccaggacactctgcagcccagggatgtggccaggacccttggagactcccagtctgtgcttgggtgtgaaccgggagtgaaatgtgacccagaagtcgcagagaaagtttcaagcagctgaggaggtgcacgtgagggcaggaagggcctggggagtgtgggaggctgggagtgagagccaatctgcctgctgacctgcagaagcctctggacttgctctctgaaagcaggatccagcgggagctggaaagaggaggttcctgtacggggccagcctgccctccccctccccactcaatgtggtggggtccctggggatctacgccctccagcggacagtagcatgctggaagggagcttggggacagatgaacaacaggaccacagaaaaccaagggggaaaaggccactagtatagaacaaacgtgtgctcatagtgcacgcccagctgtgcaggttgttcatgggaagccgctgtgagcgctggaccgtctcagctcagtccaatggagagagggagggaagggccctgagtgcttttgagtggcatgtgagcaagtgaacttcccaccttccagacaagacatagacagttgatagattccttttaaaaaagaaagaaccgaaaaaatgaattcaagcaatcgtggcagaagcgtcctatttagaaatatagaggcaggtagaagcagaaagagctggcggggttgacagctgtgtcctccggtgggggtgggatgaggaatctcttctggtagtggtttatgctctttgtagaagtggaggacattttaagttgtgtgctgagtagctctggtacaaatgaagactaggaggaggagaagaggccgggcaggggaaggggaggaagaagggggcactaggtctctctgaatttaaaccgtttaggcgtctatgtagacatttttctttcttaaaaaaatggggatgcgggtggtggctacacgggtgtgcacatttatcaaaattcctctgattgcactcttaaatttgtgcttttctccctaagtacattcacttcccattttgaagaaggaggggagggaggctttccatcttctacctggttatacgttcgaatttcgtgccttggcgatgcattgcttgtttaaaaactacatacatcatgagggaaaatctttatcaaaaactctggtcgggcgcagtggctcaagcctgtaatcccaacacttggggaggccgaggcagacggaccacttgagcttaggagttcacgacgagcctgggcaacatacagcaccccttctctacaaaaaataaaaattagccgggcgtggtggccgcgcctgaagtcccagctactcggagactgagatgggaggatcactcgagcccaggaaatggaggctgcagtgagccctgatcgcgacactgcactccagcctgggcgacacagcggaccatctcaataacaaaccgaaactctggctatctccaggtagtcgaacttgggatgccttttgttttcagtgcttgtaattttctgaatttttaaaataacgaacagagtctccttgcgacattaaatgcaatgcagccgccgaggaagcgtcggggacagatggggttggacgccgtccccacttcctcccctgcagccgcctccaggccgccaacctcgcccagccggagcctcagtcctggagggtcgggcggcggcggccttcccctggagtggcattcttgttgcccgtggggccccctccccaggtaggaggcaggaggcggggtctggggcagcctccgctttctccttcctccttcccccgcggcttccctgctgcgccgggcctcccaggtctcaccccgagtttccgcacccggtggggcgcgggttccggcccagcttctctcggaagtgacggctgacctagagcccatccctggcctcggcctccgcacccccccgcccccctgcctgtggaacccgcagcccagaagcctggagcctctctgcgagcacgcaggggtctcccagcttccggatcgttccgccgttcgccctgggtctgagtctgggggttcgggaaacaattttggacgtttctggttggattgtgtctaaatcgtgcattttacgcgacaggggactcgaggtccttgaggatcccaaactgccgcagcccctccctcctcctccccttgcccttgcccctgcccagagaagtgcgctcctccagggtcatccacacgcgccgtgccgagccccgggccccagttccaggagccgcgtctctacgggccagtgggtcctgagagagtagggcttcgggcgctcggggagaggaggcggaggaggggtgaagccgaggggtggcttttaacgaaaaagaagttgaagcatcagctaagccattagggctaatgcgatgtactgtaagacggggatcaatgccgggcctcggggaccggcgtgggggagggggcagcaggccgaccgcccccaagccggtcgaggcccccgtccatttgggggaaatggattttcgcgatttaagaaacaaacccaaatcaaatgagcgaggcccggatgtgctgacgctgcggttacgcgcgcggagctggagccccgagagcgctctaggaaaggcgcagcggcgaccgcgggagggggtgagaagccgaggcagagaggtccggagaagcagaaagagatgccgaccggcagagaaagagcgagacagacacaccgaggggtgagggggcaaaagggagcagcctcgcaattttcgcagaagaaaacaggccggcgcggctggggatgcgcttctgggccaggcgcggcaagaccccaggaactagccagagccccaagacccaggcgcctatttgagctcctttgagtccggcaggaggcctaggtccattcgggagagagaggtgccagcccctaccttgtgcagtcccctcgcccggacgtcatggggggagtgccccctgagcgcaggcccccgggcggccggggcctcgcttctggcggccgggagaggtcggcaccgtgacactcaggcacacatacaccaatgccccgggcccgctagggacgctgagctccaggcaacgaaaaacgaaggtcctggagtcggtcctgagtgcattcctccactgggccccgtcgagaatgaaactctcggagcatcgtcactgggaggtcctggggggacactggggtgggcgagcttcttcaaagactcggtttccactgggccctcccattccctcctctcttcgtcacgcggtggtggtggtggtggggggtgcaatagcccctctggttcctggggcaccccttgtggtggggtgtgccctcctggctggacccactccttgctgggtgcaggagcagagtggggaggaaggaggccggtggatttggcttctgggcagcccacctctgcctctcctggggccacactgggcttatctgtaaaatgggcacaggtgagatagagttggccaaggtctcttctagcaggctaagggtctgctggggaggcgggctcgctccaaggctggcccccccccacgcctcttgacagtgccacccccaggatatgaggaacctcaggcaaatatgggggattcttgtctatataaacagttttagtcatcgcaaactcataatgtcagcaccatcccgttagcacgatttctctagtcccgattgacagccctcccagaacctgggccttacaggtctgagctggcttagagcttccaccccaatcccagaaaaggcccgcgggacttagaaaattccaaaaataaaaggcactccacagtatggggccctctgaggtgcagcctgggggcacaagggtactgcctctcagacccccatgtggagagcccgctggaagagggagaggaggattgaggaagtggggagggtgcgagacctccggagctgacaccctcccccactcacccagaccaggggtgagctgggctcagcatcctcatctcctcgccctgtcattgccgccactagagaaatggggacagagtctctgccttctctacacacgtgcgtggtctatgtgacgtggagtcgcactgggacccgaatgaggacatccccccgccccttcgtcatcgtgggaataccgtggaacaaggcgggcaaagggctgatcagctcccgtcccggcccgaagaaagcttccaggggaggcggaagggccattcgccattccagagcaggcgcgggcagtggcccacggctctgggcccggatctgtgtccacctctggatacccaattcccgccaccatgttgttgggagcaaggaggcacaactgcccgtggtgggagtggagggcttaggtccccattagaaccggcagagccccgacggggccaacggcggtgctgacagcgcgaactgcgtcaggggcgaagccgagttcgtggagcgccgttgcgcacaagtgcgcgggtcagcagggccctggtgcctggacacccccgggtgcgcgcggcgtggctgccctggtgccccagtgagagcatgactgcgcgccaaatggaagtgggggcgccccctggcctttcctatacccggggccaaaaccgctgggaccgatatactgcaggagtccgggcacaggctgaggggaccggcccgtgcgttgagcgcctcggaggtgctccgggtcatggtgggtgctgagcgggaacacggaaggctgtgtacacagcagcaagcaatgcacccgcgctcccgtcattacatcgcgccgccttcacccccgctacgatgaccactccgcagatgggcaagacgggctcagagagatgaagtgactttcccaaagccacacagcgggccagtgaaggcaccgcgcacggtccaggaatctaacccttcctggggagtggggagggaagggctcgacccgaaggggccaggcggcagagagccccgcgtcccccgccacgggctaagacgcaaggcggcccgccgtgtctccacctgcgcgcctcgagcttcgctccgctctagccttcgggttaacaccgcttccttccagaagcctaaggaggcccgggcggatcagacgcctcccttctccgcagccccatgccctgcccgcagcttgagctcaccgcccttctcggggtggcggtgcggagaccggacggctccactgtgcagaccccgaggggtaaggggccgggtaggggaggggagcgagaagtggcgaggtgagaactgaggggcgaggctgaaggctgagccagacttccagacgccagagagacgccaggtgagccgggcgggggcgggggcgagggcgggggagggggcggggccggaaccgcggggagggggcggggcgcccgggagggccgcgagctgggaaggtcccgggcggccggagacggtgaagaggcggggccgagtcgcgtccgcggggtcccggacacagcggagctggcgccactgtcggggttccgttccatctaggtcgggcagccccgcgggtccgggcgccgctgccctcctctacctgctgggaagcgcaggccgagcagggatgaacctggctccagggccccagcggggcaggcagggcccggaccagaagctgcggcaccgacttcccactgcccgaccgggcgacgcggggcctgggaggggcgggtcgggatcagagcgccgctgcgccccggaggccgcgcttcccgcggcctgtgccgaggagcctctccgcaaagaaataaacacgccctgtctccccgaccccgactcgggccggtcagaagtagggagaatcacgctcgggaaaggggggtagggaacgacgggggagcctcggtgaccagggcagatgcacgcgcgcgcgggatcctcgtgcgccgcgaagagggacgagcagaggagcatcggaagaagacaggcgaaggggaccgcggagcagcgtaggcggagccccgggggcacggccgaggctgcgcttcaggagtgtccgccaggcgccttcccgggcggttggcgaaacccgaggaggcccacagctctggcctggggcgccgtcgttccaggggcctctgcgcctctctctggcgtctgtctggaccctcagagccccgggatcaaggcgcccatcctcttgatggcggggcgcacctttcgcgtgggtcgggcgctgcagaccaagcacgccgagtgcgtcaaggaagtgcccggaaaccaaagacaacgcacccgggaccaagaggaggccgctgcgcgcaacctcccaaacactgcagcgaagtcggaaccacgtccgcgtgggtgactcgggctggggagccgggaggcggcgccaaggacccttcagcccttgtccgtcctccctgatatgtcctcgacgcggtctgaggcctccccagcggaggcgacgccccggcgccaggtgccctgggtgggggaacccggagggggcaccacgaccagtggagaaggggcggaggccgggaaagctgcgtggaaaagaggaagggttaagactgaaatcattctacagagaccacgttaatgggtttaaatgcgaacattctagctaagtagaaaaaaatttaaaagcagagtgcaagctggaccaaagtgagaccccgattgggcgtagataaacctgcaaccaaaacagccccaggcagagattcagccgctggggagcacaaacccctaatgtttcttggtagtcactttttgaaacattttattatggaagttttcaaatataaaacaaaatcgagggaggcttcaagtgaattcccgtgtacccacccctatcccacagttcttaaaccgtgaccaatctagttccctctattggcctccttttatttgtactaacttatttttgttgccttattataaagcaaacacagacacattttcactgaatacataaatgcattttcactgataataactttttttaaaataacaatacggcttaagttacaattcccagtaaatccacactcaaaggcagttggcttttgatgttggaagtgcagacgtatttccatttgaacttcactgcaatgtccagataataaaaagcactattacttttgtaataaaaaataaacaaaaactcagccccacagctccagaacgtggcgagggccgtttgctccccaggcggtgaggctggctcttcgaccacttagctgttctcggcgcccgatcctgtgtgtccttccgtgcatgacacgcagagcttagctctgtgaggacagggctgtcccctctccggcccacctatgaaggggtgtccacctctccgtttccctccagggcagtcattcattgggtgatccgccttggggacctggggatcggcgagtccaggaggcccagcgaacgaatttccggtggctgggacccacttcccagcgggaccctggataggcatgcccactctggctaccagaagggcagggtggccgaagggtatgaagcatatgagtgcacaggcggggaggaacagcagcagcaccagcgcaggagtagcccgctggc

SEQ ID NO: 104 - DNA, 2194 bp, Homo sapiens
ttaaacccagtattcaccactgggacatttctatttgccttcacaggaagggaaaggggaaggcatattggagccagcgctcacagggcagaaccagacgagcctcactggaggcaaactgggaggtaggcgtgcgctgtccgtggtgctgaaagcttgaccggcgcgagctggagccgccaccggctgcctcggggtctcgccgggccttacctgctccgcgccctggaagcagatcttgcagatgggctggtggtgctggtgctggtgcccagcgcgctggtcgccgccgccactgctgctgctgcggctgctgcacaccgagcgcgtctcgggctggtctccggcgccccgccgctcgcgctcgccgcccgcgccggcctcagactccccggggccgcctttcgctgctgccgcctccgggaggcgcctcggaccttccccggagtcgccggccgccgccacttcctggccggcgggctgcaggggcaggggcggaggcggcagctcgtccgctcccctgcaccgcggggccacctcccctagcggctcgcttggccccgcggcgcgctcgggggtctcgggggacgcgggcagcggcggcaggtagcgcggggccgcggggaccggggccggctctcccggcggcggcgtcggcggcggcggcgggggaggttgcgggggaggctcggcgtccccgctctccgccccgcgacaccgactgccgccgtggccgccctcaaagctcatggttgtgccgccgccgccctcctgccggcccggctggcgggccgggctctggctgcagggaaagagagcgcggagggggcgggagggagaggggaaaaggagggagggggcccggacgcctggggctagggggcgggacggggaggggatgcggaaggttctgcagctgcggcggcggcaggcgcggccgttcggtggagccgccggctcggctctgatggaggcggcgccgaattcggctgcgcgtgagagccgcgccgcggaagggggggccggagaagcgagggggcgggagggaggagcggcgcggcgggggtgacggggcgcgggcgcggggtgggctgggggcgcggatcagtgggacggagttcggggttcggctccgagcgggcgggctggaagtgggggatccctcagccgcctccacgggccggccccgcgctcacgtcggttccggggcggatgacccctctccaaacggcgcagcgctgcggctctcgtgagctgggaagtagggggcaggggagaggccgcgggtccagaaaccgttactggatgggccggtgggatgtggcgcgggccgggtggggcgcgacagtctgagccgagacccgcgtgggcttaagggtgcgcgaggcgggtgccctgggcgcgcccgaactggctgagcagtggagcgggaaagggcgcgggacccgggactgtaaccgccacttccaggccctcgctccccgcgcttggagccctcaagggcactctcagggatcctcgagagccttaaaacagaagtctctggaacctgtgtcctctccctgtctgtcccgccctcgaatccctgtgtcctcctcacccgctccctcctgcagtgagcatcccgggttgttggtaaagatcttggtgcctgggaggtcggagcttcgtctcctgaaatggtttatactagtgaaccctggcgccacgttctgtggcttataatcactttcgtcgttgccgcatgaggaagcaaatgacaccgccccttaccctggaaaagtggctgcagccttccccggatcttagttttactcaccccgaagtcaatttctcggtaactccaccctgcaaaacctctgtgggactcatcttcagggcagagctaacagttttctttctggaaaaaaaaaaaaatccctcacctgcagggaactaggctgagaatcgtgcacatgcagtagtttccaaatccgtgcagtgtgagatcataaagcaccggatttatatgcggcagtgtgtctatccgaattttcactgatgtgacgctttcagtctttgacacaaagacacagagcagtcatttttggcagcgtttcatagccgaagacccaggatcagaaaggcaaaataacttgttcaaggttatagaactattaacaaagaacttctgtgttccctgaccagattttct

SEQ ID NO: 105 - DNA, 1958 bp, Homo sapiens
atgctgggccacgctgaaaatttgtggctgagagctggaccctcgtcatcgccacgtactctgtcaatgagacagggtaacccatggttactgagctccgatcagttaaggggcagagagcctccgctccccactgctctataaaagagacccagcaaagggaccctaccagcttctagctctcagtctgcgcgagggtgtaggaaggaaagcccaggacctccggagcagagcacagcagctgcagaggcaaggccagcatgtcgcccaacttcaaacttcagtgtcacttcattctcatcttcctgacggctctaagaggggaaagccggtacctagaggtgagccacccctggactgacccatctcaccttccttgcgtgttagccctaggcggcaggcaggctgcctctgctcgcagccttttggggttcgctgtttcttccctctctgctggatgctgtcttgcccctggtttcccctatcctgttctccctccttgcctctgagcatcccagactgcctgcctgccttcctctggccgctggggataccctggcctgatggagagggtctaagaccttcccgtcttctccggacacgggaaaacattacccctctctctttattcgctatgctgagtggcagggagggtgccccgctcctggtccccttttttatccccagcccttgaacttttccggactgacctatgtttcttgccagctgagggaagcggcggactacgatcctttcctgctcttcagcgccaacctgaagcgggagctggctggggagcagccgtaccgccgcgctctgcgtgagtcgaggctgcccggctcgcgggcgcccgggacgcggggaaggtgggactctgtgcggggggcagagggctcgcggacatctcggggaaggggctggccggaaccgccaggggcgcggtccccttagctaaggatcggtccgcggaggcgcgccaggagcgggagagggtggcgcgcccggggcgcaggaacccagcgcagcctaggctggaagtcggggcgctgggcactacagagcccgggaatggggcgcgcggagagcggccgcccgaggacggcgctgcggcggctgcagcccgggacttattgccccatgccctcctcccccagggtgcctggacatgctgagcctccagggccagttcaccttcaccgccgaccggccgcagctgcactgcgcagccttcttcatcagcgagcccgaggagttcattaccatccactacgaccaggtctccatcgactgtcagggcggcgacttcctgaaggtgaggcgcccacggccagccaacctagccggagggcggcacgggagggttggaaagggctggggcgctgcacccagcgtggggctgctgagcgtaggtagccagtggcacttcttagacacttcgctggtgcccgagtcggagaggcgcgttcgaggacccccacacggcgagaatctcgcccttacccgaagacctcgacccacacacatccagtggggctggggccggtgcctctggacgctgccatggtttaaattcccttctgggcgctttcgaggccctaggaacttctaaagacttgaactcgcagcgcggaactctgacgcaatccagggccgaggaaaaatgattaaaacccaacaagctcgagtgctggggtccaccaagcgggccgtcttggttagaaggcccgccccacacgtctcacatctgagcccgcctcgctcctgctggtccacgctcggcagagccgttccgagagcacagaggctgtcttcagtgcgcgggtgacccgattttggagcagtaggggactggctgccgagggggcatctagattgagataggtgggagggcaggacaagacccctaagctcactgcctcctcgattccagtcgtcagactccataagtataacagggc

SEQ ID NO: 106 - DNA, 9660 bp, Homo sapiens
gattttgttctcctagcacttctgggcttcatttttactttaactcgaaattgtttcaaggtaatggtgaatatatgttctaatggcctacacacacgcaaccacatgcacacctatttagtatatttattggaggtgctgtgctgtacaaagagataaaatgcacaacagtgaaagggaccagaatgtagagcctacttccaggagagcagtcgtggccagaaaagagtgctcctctggattaacggagtcccctggggcgcggccttatggggatgtttaagtcccaaaggaagaggctcaaagcaggttactcttcgtgaacttcccggatcctcaagttcccagctcttcagcccccttcccgccctcccgtcctcttctctttcttccccaacagctgtcattaattggaggatggtaggcccgctagatcaccatctccattcaccgccgatgccccggggctgctgcgtacagatgcaagagcctctcgggcctctggggttgaagccccaaaagtcacgcgccttagtactgctcccacaccccaaactgccagccagccttggtgttcttcagtccctgtgttgcttggtgggtgaccggtggaggggtcttcttttgggggaagaacgtcccaggcggggcctttaactgggtgactgtggtgcagatcgcagtttcagagatccctccggaaagaagcgccgttgggtagcagcttccttccagcgccgcgtcccctccttgtcacgacctgcgggcggaggggaagggaacgagcatctggccctggttggcagagtcaagccgaggagcctcaccaggccctgacaccctgcagcgtggctgccagatggccctgcggggtggcattctggccaccgtgtccgaagcccgcgctggccagtggtgtgtgtggcgcaggacccgagggcgcgcccgcgtagcaggtgccgtagcctgcgccgtagggtgctccgctgtagcctccgtagcaagagtagggcgacactgctgcactgtaggggctggggaaggcaggtgcgccgggcccggggcccaggcagggcttgccatcgcgcaccaggacgggcacagctactcggcgcggcgttagagggtggccagccagttccagcgacttgtcctggcgctgtctcttgcatttgtagcgtcggttctggaaccagatcttgacctgcgtggacgtgagctgcagcgcgctggccaggtgctcgcgctcgggcgctgacaggtaccgctgctgcttgaagcgccgctccagggccagcacctgcgcctgcgaaaagagcacgcgcggcttccgtcgttgccgcgccttgggctgctccgagcggccaccccgcacgctgtcgccgctgttgccaacgccgcgctctggcaccctggtcccgccgccgaggggcgaggccgcgttcaggccgggctctaaaagcacaggaagggacacatcagcgcccagcctaaggctcacctgagcggttcctacttagttttcaagactttcctctccctggcagccttcactctgcgcaggctcctcctccctcctactgcccccgaaccctgtcccaggacgaaccctgtcccaggactgtctccatgttttttccctccaagctctgtcacctttatatgttactgctggaacataaattgtgacaacctatggtccctcgtgtgtgcgtccttttcctaaaggtagactctagataaagagttaaggcagctccatttgcttcttagaatgtgtgtgcatgggccgggcgcagtggctcatgcctgtaatcccagcactttgggaggccgagacgggcggatcacgaggtcaggagatcgagaccatcctggctaacacgatgaaaccccgtctctactaaaaatacaaaaaattagccgggcgaggtggcatgtgcctgtagtcccagctactcgggaggctgaggcaggagaatggcgtgaacccgggaggcggagcttgcagtgagccgagatcgtgccactgcactccagcctgggtgacagagcgagactccgtctcaaaaaacaaacaaacaaaaaacaaacaaacaaaaaaaaaactgtgtgtgcatggtaaaccttcgctgtgaggaacaagtgtgagtggaggtgtctatcagattgaggagactttggaccctccttggaagtagagaaagaaggagaagaaagaggaagaggaagaggaggaggaagaggaggaggagaagaagaagaaggaaaggaaggaaaaacagagagaagcccgggtgtcaagaaggcgcaaactcggtgacaggctctcaggaggctcctgtctgcctcattttccttcgcagccccaagcgcactgcagggaacttggctgtaagtccaggagcgggagccttgagtgtctcctaccttcacccaacttttgtgcacactcttgtctttttgacgcagcgctccaccaacaccagggatgcatcccagctttcttggatgtgatgctttgtaatccaaattaacaatatatgatgaaaacaaaatatttattggctcctgacttttggctcagaaactgaggttctctttgagagatacataaagttgaggtccacatttggcttcttaaaaagtagcctctccctcctttggggtctttctggcaagcaggtggtgtgggcttcaccctgaagtcttcctttcacccagggcgacccctacagctgagactgtgcccaccaagatgcagcgttttcctcccttctcacttctctcctgtctgggcttaaaccccgttttaccccaagggtggatagaagggaaatgctgagttttcatgggatctcacgccaggagaaaatcaggagcatggaaggggtgcaagttcatggcaagacgggacaggactctaaatgagccagtgcgattggaaccagactcctaatacccaaggcactggcacaatctcctggagggggatggagctgtttggtatgtaaagtttgaggacagaacaacttccttcagagacgtctgtgctcagataaactgaggccatctccacttgcaaagaggaaaaattctgaggagggtttgttttcttagcgcatcgaaaggtaacaactgaccttcagccaggctgcagagcaaggaggaccccatttcaaagtcacagatcatgcaggccctggggctgcacagcaccgcagggtgggccagcaccctgcgcccttctgttcacaatcctggagttcttgagggcaaggtggttagctgcgatggaggaaggcatgaagcccaccgaaggtgtcttccctgtccggatcgctaccgcggccttgggctcagttcctggagccggcctcgggtccggcctcgggtccctcctcggcggcgtccactgctgccgcagaagagacgctcccaggatttggtttccagtgggagaaggaaggcgccgcaaataccatcggctgcgaggcgactgtggtggtcgaaggaacgtgatcctttgtcagctcagcggagccgaagtgatcgccgagttggctgagcagcgccgagggagtttagaatccgctgcagaaaacctaccagcttcttcgcggacacagcaggtaatgttcttaggcttctggagttagtgagtagccggcctgcaggccggtaaataactccgatctagccccttggtttatttctttcgttcgcgccttgaatggaggcgccatggcacagtctggcagaggaggcctgccctggcgggcgctactcagggcccctgcgttgagaatgccgagtgaggtgctgaggtttggagagaaaaaggtggcgttttcatcccttggaatatcgggcctctccagctgaagcctccggatcgacacaacacaactcccggatcgacgcaacacaactccccgccagccccttcccttccagcgggctggacacaggtgcgtgggcctcgcagcccggcgcgcacggttgcgcctttggatacctgcgacaggactcggccagaaccgtgtcccagggcagagaaaaccctggcaacctttaacgttctccctgacgcggagaatcctgcgccaacggggccttccacttcgggagactcaatcgccacccggtccccaactttcgtcttccagaagaaaaatccatgaggaggaatgggggggtcccccgaccagaccagcaagaaagggcccagttaggagtgacctcagcgccacgagggttccactgctttgatggccacacgcttttgcaaacggtttgggggtggagagacaccaggtgatggtagggaagggcggtggtgactggcacggctaagacactgcggagggtttcgctgtggagatgagaaggtggaggttctgggcactgtggctgcgagtgtgagcgatgcgtttgtgggaatcagaggagcggatttgggaaaagtgaatccttgagattcagggccttactttcgttaggggtgtgtgaagcacactgggtgtcaggagaggatgggaatgggattcgagaggcctttttttggactcctcgagagaaaatggagagagtctcgaacccaggagataggaggcgtattttccccatgcacccatggatcacgcccccgcccccacattccccccgtaggaggcaagacctgagcgcttactcacgtggctcccccatccgttccgcgtccatctccaagactgcctcacagggacccccaggaggctccgaaccatccagctttctgtcaccgccgccgccaccagcgttgtgaacctctgaccctcgcggctctgcgtccattctcaggtactgaaagttttccgggctcttccgcacccgcggatgtggcgaagccgcggggcagctccgctcgcgctccagtcgcaggatgtccttgaccgagaagggggtggaggtgacggggctcagcagcatcccgaaggcggatggggcggggccgaggaggtccgggtgaggagcggcaccctgaacttcccgtcttgtcgctgcaggccccgcagacagacccaagctctgggacagacgcccagcgtcccagacagcgccttcctctgggccatgctggtaggcccgggtccagggccgggtgacgagaccgtagccccccattggttctcgcagaaaccacgtgctagctctgcaccttcctcccccagcgctttcttcccgcgcccgtcgaatcctctccagtccccagtcgccttcctgggaggtttggttcgtattgacggttctcaaagcaaagaaaacgtggagagacagtgctgcgcgcggcttggatctccgtcaatacggtactagcgggagcttccgaggtcaacctagggtccacggggaccctcctagtggacatgcggggattggaggtggagtccttgctgtgcaacccgatccggtccatccagcctggtcttagctcaactctgcattcttatggcggaggaaagctaagatagagacagctgggacccgcggaatttgaggcgcgcgctgggatcttacgtgcgcagaggccggaccgcagagtagaagtgagtcttgacccaccagggcctatattgagatggaaacctctctctttgaacagaatccaggtctgcggagtcccgacccgccacttcctaggagcagcttctcagccccaggaccgaggaatgggtgtgggggctacatgtggggagccctcgacctcttaaagggctccgcgaatggtgggggagactggaaggcaatctctgcggcccgcgcaaagtggtcagaggcgggagtgggggtgggggggggtgggcttccgtcgcccttggcgtccccacgttctttttcttcctctcctttatcctccttaaagcggattgcctggagcgtgccagcttgtgtgcgcgtataaatgcggcgcccgccgaagggctgggaagatttgactcgcattagatctccagaaaaggcccttggttacggcgggcaccgagtaaatggcgcagtgtggcacaaaggttaccaacacggagatagcgccaacagatccagcccctgctgttactactcctggatcagggaaacgtgatttggtgtcagctgttccagcactgaatcgaagagggtttaaaaattgtgttttaatcacaatgggatctcattagagttcttttggggatttcagatcaaatgtttctttttgtttttctttccttcctttccctccctccctccctcctttttctttctttcttttctttctttctctttcttcctttttttttttttttttggttacagggtctctctctgtctcccaggctggaatgcagtggccggatcatggctcgctgcagcctcgacccggactcagtgatcctcccacctcagcctcctgagtagctgggaccacagatgcagccactaagcctggctatttatttatttatttattgcttgtagagccggggtttcgccatgttgcttaggctggtcttgaactcctgggctcaagtaatgtgcccacctcggcctcccaaagtgctgggattacaggtgtgagccaccgcgcaggcttcaaatgcttcttaaacctacttacgtgaattcaatgttacaacagctgtttgaatatgattatctcctgaaattcagataaacccaactcatctccccaggctaatgggtgcaaaattgcacacaaatatttatgcaaaatttcagcacatttgatcattcagctacaaagtaatgatttcctatacatggtaacatttaatcttcacatcaaagctatacccaaatatttccagctgctgctccttcttcttcttcctcctcctcttcttcttcttcttcttcttcttcttcttcttcttcttcttcttcttcttcttcttcttcttcttcttcttcccctcctcctcctcctcctccttctccttctccttcttcttcttttgttcagcgaggagtccagatcagagaggtggcccagatagttcaccaaatggcaagaccagactagcctctaaaccaagacaatttaccctgtctttctttctttttgaaatatacatcctttgctcttagagatctcaaaatcttctgatgacccatacatttatttgaatttttgtttcctgataaaagtcatcatgcgcaccttggaaaaagtgaagaaaagttaaaagaaaatatccacccagagacaacattttagtggaccttcttgtctttatttctgtatatatgagctcatagggtatatatgtttgtatatgtttaaatttacatcataagcatgcccagttcatctttagaattttttaaacaaatataatttaaaataggaatctaatatttcatgttattgatagaccgtagtagactgtgtttgtatttttagataatatctttttttatcactgtaaataagactttattttgtgccttcaattttcatccttgtcaatattttctgttattttctcaggatacatatgtagaagaaacatcaatcagtcaaatattttaatgctcataagacatgccattccagggcttcctagaaaaacagtattatcgtattttgttcccggaggtgcaggcgagtgcctggtctacgggtgtttacaatcatcaccgggcacccacaagagaccctgaacttagaatatttccgcggtaggagttccgcctcgtccttccaggggatactgacgactcgctgggttctcgccttccaagtgctggaggggggaagtagggccacccggggtagaaacctaacgccagaaaccttccagcgccttatcagttggtggaattttcgtggcagacagtcgcgctctacctaagtcgtagaagaaaagacaccttgtgggggacttggcgtcggaagccttgagttcaaatcgtagccctaggtccccagccttgcccttcctggcttgctgcgaggatgaaatgagatttttgtacgtgaaaatgctttctaacccctctcgaggggtggaaacgtctccattcgaccagagtccgggtctgcggagcaatttacccgccgctttccaagagcaggctccccagcgtagcgagtccttgttatggaaagggtcgtttcggctcaggatgcgcgctcccggcgtagacctggggataggggtccctgtcgcgctcgccccacccctgcagggagcaaggccgtccttctgcagcaccgcgccacgcggcgggaatttgcagtgtcctgctttctggtttcagcttcttcgctcgcttgggtctctctcggtcacctctcggaggggcctggagtccctcctccctcctcctacttcagcgccagaagcattgaaagccgcccgcccgccgagagtggaaccggtacctcgaatctcgctcattccgggtcaattggaggcgcattagcggctgccttgcagtgcaacagaggctcgagtgacaggccacctgcctaagcgctggcgcccaggaaagccggggaggaaactaggaagggaggcgacagcagaaaagccagagagtccagagactcctaggcaggcctccgtgcagccccaacagcgtggtgccgggaggtgtgtgtgtgtgtgtgtgtgtgtgtgtgtgtgtgtgtgtgtgtgtcccttactcaggaatggagacatcgtgatcacctctgttcttaggtgagatggagacggccgtgctaggggtgtgattgatccgctttgcttcactcaggtccgcagagtgttttaagcgcctaccgtgggccagctctgcccaagctctgggaatactgagaaaatgagaggtctcagccttcttatccgaacttgcattcttgcatagaagacagacttttaaataattcccatacattgtgttaccttgcacaatcagacggaatttgtacactatatttattgattttttttttttgagacggagtttcgctcttttgcccaggctggagtgcagtggcatgatctcggctcactgcaacctccgccttttcggtttcaagcaattctcctgcctcagcctcccgagtagctgggattacaggtgcccgccaccacacccggctaatttttgtatttttagtagagaccgggtttcaccatgttggccaggttagtctcgaactcctgacctcgtgatctacccgcctcggcctcccaaagtgctgggattacaggcgtgaaccaccgcgcccggccctgaacctctacttctccgtggttaacaaaagtcattatttcctgtttctttcactctcctgaaacgaagggattccctccagcaagtaccatactcaacaggcaattcctggtaacctcgcggcttagtgggccctgtgggccgaggtgaaatcagtcctctttaacccaagtgcaggatccccgttagactagactagacttgggctctgggtctgttgtcacagctgtgtccccaaggcacgtagtaggtctttaacaaatatttattgaggacaggtatctgtcctgcttgattggggatagttcctaggatactctggggactgtcccaaagggtgtctggggaccatcaatggtggcaatttctgctttgccacggtcccttttgctggga

SEQ ID NO: 107 - DNA, 1451 bp, Homo sapiens
tttttctttaaaaagactattcttttccatatcaacgatttcattttggaggcagtcgctgagataatctcaggtgtaaaataagctaattaaacacacgccacttaaatactgagattagctggagctcaggttttcagagcaaagcaagagcacaaatcaaccccgctgagttgctgctactcctgagataacccgaccccatcacggagattcccaggtggctgcgaacgtcaacgcttagcaggaacgattaagcgattcacagcactcaggggattgtgtaaatgcccggcccgggcgaccagcagggacagattcaactccccgggcgcaaagtcatctccagcccgatcctcgctccccctgaaaacccaccgctcgtctccccgggagctgcctccgtaggagaatgttcgcccccagccgcgccccaactttgccttgcgtggtcccaaactttccctggtggattaataggaaaataaatctaaaggacagacgccccacctgtgagcttggagctgggttgcatttggtgaggcgcttggcccgcatccccctgagtcgtttgtaggtggctggggaatggaagcgcccctctgcgcgtctcccccgacggcaggccctgccccacgccccccatcccaagccaaaagcaagggtaggagaggcgggggctccaaatccacgccccggagcacagagagttggctaactcctagcggggcctggggcgcccacatccacggcagagccacctgtgagcctgcagagcagcaacgggatgcgcgcaaccgggatgcggggacgaccgccacagcggctacagcggcaggcggggcgcagccgcccgcacttaccaggataagatgggaggacgcgcacacggcggggcccccgagcgcggcccgccgctccagtccgccggccctccgcctacccgctctgaggcccgggctctggctcgcactggctgccgctctcgccagccgggctgggttcgggaggagactgagccgctgtgagcccggcgctccgagtctggcgctgcccggcccccgccggcccctccctctgggctgtgcgctgtgcgctgggagcggggccgcagcgcgctcagctcccgagtcctttgctccacgcctcctgggcgcagaggcgacgctggcagccgtctcttaggagatttctccgcagtgtggacgggggtcaggatttgcaggcgagcaagggcagagaggagaaatcgtacaggtcggtgacgcgcctaattcacaaagcctgaaagtgctgtcaactttggggtcatcttatgctgggcaccgcctctccaattatctgcaaaaagtgatagcagtcgcgcttttctttaggaggcgatgtgatgtgtaatggtggaaaagacgttcaatgaaaaggcacggaggcctgcaggacaggctgttggg

SEQ ID NO: 108 - DNA, 1242 bp, Homo sapiens
ccgaataaaaaatataaactttgacaggcaaactaagatgctttcatgcattttatgctaaaaccgtaccaactctccgtaaaactctttacctgcgttcgccccatcccctggttcgcattgttctcagtaatgaagacctccttgcctatcactaacaccaattagcttacaccaaacttcaggacgccaaggaaaaggaaaaatcactcttccagcagcggtcatttaagaaaaaaaaaaattgataataatgtggaactccctccaaaattagcctacaccgtgatataaattggttttctttgtgatctaaaattgactcgcagttgactagttagaaaatagttgctgaaaagccggggccttggagctttcggatcctgcccgcctttcatcatgtaaacaaacgcatcagatttaaagctttcccataattgttatgctaaccttggagcgcaacctctccatttgcatttgaaggagctaaatattaggcaggaaagaaagtgctctttttgaaagcctgagaaaatgtccccgctcggggctgctccgccatctgggccgcgggctgggcgcgcggctcccgcccccagctccttggcagaggcgccggaggaaggggcgccgcgaagggccgtcatcttgttggaaaagaatgcagaaatgcccccctaaggctgaatgagcaccacttccacactcagggcgggggaggccgggggacgtgggagcggcgcgccaggagcgaggcgtccctggtgacagcgcgtcccgagggctctcccttttcccagagcgcataccaaaggcagactcctttgcaggctccacgagccccattcgcggtggaaaggggcgttccagactgcagggtgaggggctccggtgtgtgtgtggtgtgtgtggtgtgtgtgtgtacgcgcgcgcacgcccttaaggaaaaatccatcttttataagtgctgtggcccacttttaactggatctcgtggagtactcagatacagccggtatagtgaaggctaatagcatcagcacactttcgcgcattaatcttggagtaattaaaaataaaaacagtgagctatggggttttgttaaacaagggagaaaaaaggttccaacctcccatcagcctattctgggggaatccagagagcccatgcatccctctacctttccatgcaacctgcacacaaagcatgtctatcgggagtcacggggaaaatcattcctg

SEQ ID NO: 109 - DNA, 1206 bp, Homo sapiens
gagagcagcactcatttcttcttatcatttctgaactgcgagacaaagaactttaattttggggctagaatttaatttgtttttgagaataccgaatattaccaagcaacgccagcacggatataatagtagtgtgaggtttgcacacacgcagctgaaatggtaaatttaaaataggagacactgtgtgcacgtatagtgtgtgcatacagtatcaagacattctttgctcctggagttttctctgctttgccaaacttcacgccccttttggaaagggggagcgaattttccaagttgtgaggatacttttcctccatccagccacaccctcttccgggccagtctttggtgaaacctgggcgatgtggagtcactgggggatgtgagtgagggcgtttttgctcatcttaacccaggtctctgggagagcctttaggatcgctctgggacttagaggaggttccgtcctgggccgggagctgaggctctccggacaaacgctaagcagctctcacttatcggtgtccaataggccttatataaagtgccaaagtgccccagcaaggctttaccagcacctgggccgtagttgtctcctggctcctggggtccgcggagctctagatgtacctgcagctcctcccgagtcctgcaagccacccttgtccctcttctcccgctcaccccccggcccccccatctcttttgctattccggggaaggccacgcagggtgcaacccggacgcgcccccgggggaagcccgcgacgcagcagccacacccccagcctgcactggcgcccaccccatgacttcgccttcgcggtcgcgggcagggagcgcgcgtttccatcggttgattagaagcgggagtcggaggagcccccgcccgccggcggccagcgcacagcccggccaggggcgccctccctgccgccgccggccctttgatcgcccgcggcccgcggcccgcagaccacagccagcgctgtgctgggcaggtggcggcagcggggcgcggcgcgctcaggcgcacgggtccccgggccgcggcgcgctccccgccacgccgcacatcaaggcccggccggccggcgggcgccttcattagcagcctgaaattataatattatgttaaagaacaaagctgctccccggaaaaatatgtgctgcatatctgacaaagataaattggattattca

SEQ ID NO: 110 - DNA, 2308 bp, Homo sapiens
ctagttctaaagtgcagtgtggcattagcgaatgtggtggactccggcaggcctggggcaggggaatctccggtaacatccctcagtaccgaaggcaggatttcccggccacttccgtcggttggaattctcttaagcattgccctgagtctattttcaaatgaaagggggaaaaacccaaaccagattagaatctgaaacagctgaaacagctatcgatttccagcttgctttcaagtctggcagagactgtgaaagtcctaacttctccgcgatgtcagccacggcccgcggcggtcacggagccacttcagcacctcgcgatggacagtgacccccggcgtggcgcgagttccctgcggtccccacgcgggcctgggcggcgggtccctccacggctccgtccagacacccggaggcacctgccaaagctgcggaggtggacgaccgggcgtccaagcacgaactttctcgccgaggcacggctgccgggctccgtcctgagggtttggggcggcgaggaccgccggggaggagggcgcgcgagccggaggccagagccgggccgggcagggcgcgcccaggagcaggggagacggcgacccgggccgaggcgggacggggacacgttacctgcccgtggagcgcggggaaggggtgcgcggcggcggcggcggcggagtctggcgcagagggcagcggcagcagcgggagcaccggctcgctcggggccggccagccggggagaccggggagcagcggggccgcgggcggcggggccgaggcgagcagcaccgggggaaccacgacccccgcgctgagccggagcagcccggccgagcccggcaccatccagacccagggcggcggcggcggcggctctgcgtccatcggccagaggagaggctgccgccgccggggctgctgctgctgcagctgccgccgccgccgctggaacctggcgcccgcctgcatcctacatccccgcgctgacggcggcggcgggggcggcgggcgcccgagatgctgggccggcggcggcgcggctccggagcgaactcacttggctccgcggcgcggccgctcgctccgcccctgccccgcgcgggaagctcctcctcctcccgcccgcgccgccgccgcccccggacccggcccccgccccggtccccgcccgcccgcgcccctccccgccggaccccgccccgccctcgcgctccccggccctggccggggtctcctccccgctgcgcccctggacccgggcggccgcggaggacgtgggtgggagagggccgagggcgcgcccggctgggctcccgccgatgccggccccctggtggcctctggccctctgggacccagcgcaccgcgagggaggctccgggctccgcactcaagggcaagcggaagccgaggtggcggcgccggagccaggcatgtcccgctgcccagcgcggggctgagggcgcagacggggaccgcgcgagccgctctctacctggcctcctcggagtccagggcgcggagggtactgggctgcggatccggcgcaggcggccccggacgctgctaggcgcgggcggatttctgttgcacaaatgggccaaacaggttcgtaaggcccccgcgggtcccctggcccggaagttgagccgcgggtgcctggtctgaagttccgcgccccacagtggcccgcgggcctcggcggctcctgccctacgccctccaaggcaccaggcaggtggccccagggcctttgcccacgacaccccctgggtggggcaggaccacccctctgtgaattcgtcctggatgcccccaggcacagtctgtactcctccctcccccagcacacgcggcctctgacgctgcacgccgagcttcctgggacccaggacgaaccgggctccaaggaccctcgctccgcccggaccacacgcaggcagcgccggccgagccgagcctgggtccgcactggccacgcgcagccaccgccagacggcgctagccctcccccgctgtagaccctgcttcggaggccgccagcctcctccccatcccgggtccacagcttcagtgcagcccctggcgacgcgcgctgacagctgggcagagacttccgtgcgctgtaaaccccttaggtcgctaaacccaggaaacccaagcacagctaatgccagcgcgagaccagtgttgttagaggcatttgaagggtctttctggatgcaaaaggcagcattatgatccatgcatttaagaacttgcatgaaaaaccctccctgacctgggtgggagagcaggcacc

SEQ ID NO: 111 - DNA, 2201 bp, Homo sapiens
tctccctttctgcatgtaaccccgggcgagggaaagcagaacttggggggagactgtaaaacccagagtaaggtggaccctgggcagagtgggggagctcacacctgcaagctgcgtgcaaccgaaaggctggcgacctgtagcttgtcagattcagatatggtgcaggtgagggcctggcaaacatttagtttaatttctgagaccgttagagacaaggttaaattccaggtgaacgcggttccagagtggctgggattcccactctcagatgggaacccttcagacaagagtaaaacaggagggacttcttcgccgcccgccctgcggtgtgtgccacggaggccgctgggtgggggcgtccaaatgtggaagcgttcctccagcatgcgtggatatcctgaggactcacggacaccgattccctcttccgggcccagacgtcctaattcccgtcccccttgaggtgagatcccgttttcccccagccaaagggactccggagccgcattcacctgacttggctgccagcgcccgccttcctcgagttctcagggcaaggggctgtggtgcattttctggggatggtttgtggatgtggaagagggaaatcctttcagggattttagtcatttggcaattggtgattttcatatgacgtgctggttttaggggccaactctgataagctgggacgccctccccttgattctagaacagaaggcattttgggctaaggacgctcacaacccggtttcctgtcccgcgtggttcatggggcttgccctgggcctgggaggccggtggcgggagcctcccctacagtgaggtgggtggggggcgccacacggcgtctcagggagaggggagaggcggcttctgttccgggaagagggaacggtggaggccgccgttctcttgggcgcggcctctgctgggggacggcggggatcgcagggccgaggggccgggcgcgcgcgggggagggacccaggccaggggccgctcgcctcggggcggggtctctggagagcgcgcagaggggacgcttcgtgaatgcctgccggcctgaaggatgtcgctgatctctgcccccttcgccaaggccgggccaggcgagtaggggcgcggcctgagcccctgcggtgattccccaggtgtggaatcaaaacagatcccagagcgcgatccgcggggcttttggaaggagccgcccagggctggagattccgacgcaaagacccggaggcccgcggagctgtgggcgcagctggagatggcggaggccccagcagggagtgggggctcggggtccgcagaggcagctccatccccggcgctgggggccgcatggttcccgcaggctgtggccaatcgaggaccgggctccgcattcttcagacgttcctgagccccctcttgccccaacgtccccctcccgggcagccccacagtgggcgattttcccgcactccgcacgagtccatccccatggtggactaggggccgggtctaggctctgcgctacccggccagagcgtccagcggccccactgggcggcggcgccacgcggacgtgggtgggaacagcccccggacccgccgggagaccctcgcggggaagagaagccgagcagcttgggggcgccccggcagggagggacgtgcggctgggaagagctggcaggtggcacccgacccgcagcctggtgagcaaggtgccaactgtccgcccaatttcctgtccttgcagtaaaactgtaaagggagacatttagctcggcaggaaactcagagctaaattttgcaatttaattttgcaatttaattttaattaaaaacaaaattatctttttttaatatgtgtatatatatagagacagcaagcgaactgatttgggaactttatatatatacatatatacgtatatgtatatacatatgtgtgtgtgtgtacatatatgtatgtatatacgtatagctgatttgggaacaaatagctgctcactgtgtggaatttctgaacattcacaaaagttatttaaagaaagtaaaggccgggcacggcggctcacacctgtaatcccagcactttgggaggccgaggctggcggatcacttgaggtcaggagttccagaccagcctggtcaacgtggtgaaacaccttctctaccaaacaaacaaacaaacaaacaaacag

SEQ ID NO: 112 - DNA, 3047 bp, Homo sapiens
gcatcggccttgcactaaggcttcatcctttcactccctgaacagctagcaaaacacgaagacaaggatgggaggaaaggcctccctccggctttgacgccttgctccctcacgaaccgctttcagatgtacggatccccttcaggcaaaaagaaaaacaccactaaaagaaaccagtcacccccatttcagtgtgcagataaagaacctggagttagaccctttcctccatcggctggtctctagctgtggttgggcactcccgaaagatagatggagagggagccaagcccccagggttagggcagtagctggccgggcggagctctgccaggagcgggtctgggagctccaggatgcctggggcgtgagcggagggagggagtaaagccgcgctcagttccctctctgccgctgcgcgtggtccgcgctcgggctcgcttgccccctctctagcccaaagccccattcgcagcgcctgggctggtcccactcatctgcgccaggtggaggggaaggggaggaggtctaaaggtgtggagtaagggagggagaggagaaaggaggaagggagagactgggcacttcaagggtcagaggaggaccacctgtggtcacagccatcgtcacgaccccctgccgcgcccacgcacgcgtggatggggccaggctctgggtgccacctgggaggagcatccattcataatccggatctgcccccgcctgtgccttttctcctcgtaactcccaccccccatccccgaccccgggtctctgagctaggtttcttaaggggaaaacatctctggcaccgtttgtcctggtgaaatcaggtccctgctttatccagctcagctctgaataataattattgggaggacaggaagcaacaaggtaaatcaagaaagtgacaaggaggtgggcgcagcggctcacgcctgtaatcccagcgctttgggaggccgaggcgggaggatcgcttgagcccaggagttcgagaccagcctggccaacatggcgagacccgtctctactaaaaatacaaaaattagccgggcgtggcggcatgcgcctgtaatcccagctacttggcaggctggggtgggaggagcgcttgagcccgggaggcagaggttgtatgaaccgagatccaaaaataaaaagaaggaaaagaaaagaaaaaagaaacaaaaaagcgaggaagcaagaaaagcaagcaagcaagaaagaaaagaaaagaaaggaagaagtggccactgtttctgtgctgtcgctgtgctattctcggcacacctgggcccaacctttccattccttacctgtgttttcttccatttgttcacgtctagaaatgtattcaagtgaacaacagtctgggatgtaggtatattatttaatctacactgatgtttagcacagctatttgtaatactcaaaaaattgtaagctacctgtttcacaatagtcgattgattgtaaatcatggtaattttttttttttgatacgcagtcttgctctgtcgcccaggctggagtgcagtggcgcgaccaccgcctccctggttcaagcaattctctgcctcagcctcccgagtagctgggattacaggtgtctgccaccgcgcctggctaatttttttgtatttttagtacagacggggtttcaccatcttggccaggctggtcttgaactcctgacctcgtaatgcacccgcctcggcctcctaaagtgctgggattacaggtgtgagccacgcgcccgaccaatcattttggtcgactttaatgggcacgtttcaagtcattttaccggagaatactatcaaaatgaaatgctcactacacactaaatggggggaaagctgtttacagaacagtttctggtgaaaagagaaaacacgtagacataatctggatggcggagactgaagagtgattatctctaggtggctggatgctgggtataggttggccgcaatgttatttttgtaaggctaggagaaaaacgttattttaaatatacggaatttgctcctctccaaatccactctccctttcgcctccctagaggttgtcaggttcaagaagcggcccggagttgcaggaagggcgccggcgtcactggccccaagagctcggaacgcgcgcgccgcaggagtgccggctgcggggtcgggttgagactggcgggaccctcggcctctgccggggtgcggaaggtggatgctacgggcaaaggggcggggcttgcggttcccagatccagaggcgggttggggacgtgagccggcgtccatgtgttctgcaccccttctcgcccggtgcctctctcaaggcacgttttccaaagtgtgttgaattcgggaatcgatcgaaaatttcaaggccaattaaatgccctctgatgtagagctccgattaggcccgaaaggcttcaaacagcccctctagaccctcgagggtcttcgccgcggtaaccttaggcgtcccctccccgagaagtctccctgaggctttcacagagggcgggaggggctgcgctggggcctccgttcccagtgcccctgactggtggggagggatggccttagtgtctgagagcagagaccagattgactcccacttgagaccagaaaacgaagaggaagcggaacccgaggtggctgcggctcccccgggacacaaccgggagaggaagtcctggccatctgccggccccgcctgggggcatctgctcgaggatccctgggagccgttatcgtattcctcagaatctgccgtgctgtccctccgcccccaaaaccaacaggacacccattctgtgattctcttcttctccatcgcccagttggaggctgctggggaccaggagaaaaaaaatgcctttcccgagttctctaattcaaatgcttatcagctttgcacaccattcggcctttccaaaagggaccagaaaacaaagatatgctaatgacaaaggataattgagacctcgcctgccttgcatattttgttcatagaagctgtagcaataataatgtaatcct

SEQ ID NO: 113 - DNA, 7316 bp, Artificial Sequence
  Synthetic
gtattgttttgttttttattgttaatttaatttgtttttaaatattatttttttttcgtagagtgttgaagatgaagagagtttagcggtataggggatttagagtttgtttttttgttgatttagtagtgatttcgttgtttgttatgattgatgtttttagaagggttagggtttttttaatttattttttttttatgtatttttgtttgatgtaatagttttggggatttttacgattacgttttttttttatttattgtttttatttttatttatttatttatgtattttttagattttattatttaggttggagtgtagtagcgcgatttcggtttattgtaacgttcgttttttgggtttaagtgatttttttgttttagttttttgagtagttaggattataggtatgtgtattacgtttagttaatttttgtatttttagtagagatggggttttattatgttggttaggttggttttgaatttttgattttaggtgatttatttatttcggtttttaaatgtgttgggattataggtatgagttatcgcgttcggttttattgtttttatttttaggagtttaaatttataatttgatttattggaatgtttataggagtttatgaattatttgagttatatggatagtttgtatgagtttaaggttattgtagatattatgttttagataggcgtgttatagtgttaaagtttttgttaggtagtttgtagaatagtttaggtttcgtgaattatgttattagaatattaataattaattttgtttaatgataaagtttgaaatgattgaaaagggtaggatttaggtaagtttgaaagaattttttatgcgataataataattttattatgaataataataatgattatgtattgaatatttaaatttaagttatgcgtttggtattatattaagtattttttttatattttgttatttaatttttataggaattttgaggttagtattattttaattttatagagtatgatatgacgttttagagagggtaagtagtttgttaaatattttatagttaggaagttgtaggattagtttgtgtttttaaggattggtttttggttgttatatatagaattaagtttggtggaggtgttagaaatagaggtttatttttgttattatcgatttttttttatatttttgtaaaggaggaaattgagaggtagggattttcgtagagtaaggaatttaaattgttgttttttgtgatttatatattgtattttaagttgtaggggtaatttaaggataaagttgtaatttaagcgggaatatatgttttatttggggttattaaaattttattagtttttttagttggtggatcggttaatttacggttatagttttttggagttgggggaaaggaaaattagggcgttttcgtaaattagtttagagagaggtttgcggaagggttcggaagtttgtaggtttttttgtatttttaattttatcgttattttggatttttaagatgatttggtttaatagagttttgtatggaaaagattggaatttagggaggagtagagtttcgtttaaggttatcggtcgagtttgaatagaattcggttttttaggagttttgtttttagttgttttgtttaaataaaattttttaggttattagattttcgtattttttggagtgggattttatttgggattaaaggagggttggtgaggggagtggtaggagggaggagtgtttcggggtttcgagtaggatgagtttgaggaagagacgggtttttatgttttttttttcgtttagataatggaggtgaattgaggggagtagagatttttttatttttagggtgggattttgagggattaggatatttttgttaggggatgtttttttttatttttgtataagttttttaaggatattttcgggtttcgaaaacggggggagggggacgacgttttagaggtttttgagtttttggtttttttcgattttaagggtttttttttttcggtttttaggcggcgacggcgggtagcgcgaagtagtaggcgtaggggcgttgggatggggatgtttttgtaggtttaaggttttttttgggagtttaaataaagattacggtagcgtcgttttttttttcgggaattcgacgtcgcgcggttatagggggtttggaggggcgggtagggtttcgtagcgtatttagtatagttcgcgcggcggagcgggtgagaagtcggcgggggcgcggatcgatcggggtgttttttaggtttcgcgtcgcggttttcgttcgttttttcgttcgtttatcgggtattttagtcgcgtagaaggcggaagttacgcgcgagggatcgcggttcgttcgggattagttttaggttcggtatcgtttcgcgggtcgagcgtttatattcgttaaatttacgcgggtacgttttcgcggcgtatcgtttttagttcggttttcgtttttgtagtcgcgggtacgcggaggggtttttggttgttcgtatttgtattcgcgcgtcggcggcgtcgaagtttcgtttttcgtttgcgcgtttgtttcgttcgtattttcgcggtgagtcggcggcgttttcgtttttgagtttagggttagtttttttcgtcgtcgcggttgttgcgcgcgttttcgtttagtttagtttagtttcgagtacgattttagttttacgtacgattttagtttcgcgagtttcgtatcgattcgttttcgttttatttcgttttcgcgggggcggcgttttttttttttcgcggttttcgtttttttttttcgttttttcggtcgcgttggggatttttagtcgtcgttcgcgattttttatcgcgacgttcggaggcggcggggtttttttgttcgggcggcgggtacgggggattattttttacggtgttatcgtatttatttcgcgtttttttttcgttttttggagtttatcgggattaggtggcggcgggtgtttttttgggggtgcgcggttatgtaattggtggatttttttaaatcgttttggaggggggagcgcggcgttgggggcgggagagcgtttttggttgtgagttgtttttgtcgtttcgtttcgcgtttttttgtcgtttcgtttcgggtttttcgcgttttttttttcggttcggtcgagcgcgttgtttcgacgtcgttatttagagtcgggtcgcgtcgggcgtcgagatgaaggtgttgggatatcggttggagttgtttataggtatcgttcgtttgtttcgtagtcggtcgttatttttcgagttggagcggatttcgggcgcggcggtcggggattggggcggttcgggtttgagtaggaaggggtgcggattttaattaagttttagttttgtgttatttgtttgtgtgcggagtttagtttcgggagaggatttgaggttgtggcgagtttttggcgttggcgttcgggttgcgggagtatcggttagggggtggttttatggggtttttgattagcggagttcggattaggattttgaaagttagtttagggtttttgttttttaattagtgtcgtttgttttttaagaaaggattcgtgggtttttggtaggattcgcgttatggattttttatttttgcgttttgtgataatttgagtcgtttttttttgggggagaagttttttgttgggagtggaggcgacgttaagtggtttgggaagtgggaagttagattggattttattgattggggatttttagttttggggtttttttggagaagtgattagttgttttgttggaaatttatatttagggggtagtggttggagagtaagagcgaacggttaggaagaggaggtgggaaagggagtagggacgggggggaggattcgaggagtgatttttgtgtttttttcggtgtggagagatttagataggaggaaaggaaagtaattcggttttttttagttttgggatttataggtgttttattcgtgtatgttagatgagtatagattttagtaagtgtttttcgatatttgggggagggggttgattattgttttttaggattttaatgttcgatgagggagtagagtttcggagttttgttataaggttggaaggggtagtcgtttgtgggtgcgtttaggaaacggtggaattcgagtcgggggtagtttttgaagatttcgaaaaataatttttgttaatgaagaaggaggtggtattatgggtttaggattaagggaatggtttatgttttggggtgtgagagaggtttgttcggggagaggtatggaggttgaatggtttggaatgtgcgtgtgtgttgcgggggagggggttagaaggtgggggtggggtacgttgggtgaggttttattaaatcggtagtagtaggttaggcgtagtggtttatgtttgtaattttagtattttgtgaggttgaggttagaggatcgtttaaggttaggagtttaagattagtttgggtaatatagcgagattttatttttataaaaaaataaataaataaattagtaatagttcgtgatagatttgtagagaaggaaaggtggaagttgtttttgggatattggaaattgattgggtgtttagaagttttagcgttgagtttagttagttacggggttggtattttttttaattttttgttaattttttttttgtggagtttatttgaggtaggtttagtttatttaatttggaagggaatcgcgtaggttattgaatttgttaattttttggagggaaggtttgcggttttatgggtgttttgtttgtttgttttgttttgagatggagttttattttgtcgtttaggttggagtgtagtggtatagtttcggttttttgtagcgttcgttttttaggattaagggattttcgtgttttagttttttaagtagttgggattataggtacgcgttattatgtatgtttaatttttgtatttttagtagagatgggtttcgttatgttggttaggttggtttcgaatttttgattttaagtgatttatttgtttttgttttttaaagtgttgggattataggcgtgagttatcgtatttagtttttattatgggtttttagagttttgtatggtgaatagattttttggttttttttttttttaattttttttgtttttaaagggtttggatgagaaatgtttataggagatatgtgtggggggtttaagtaggggtttaggttttggagattttatgtattgtttttttattttttggatagaggagttaggatttttttttttttattagatggggttattagattttggcgggttttttaagtttttggttagggtggatgaggttttttttagttattgttattagttataggtttcgtttttattgtatttgtaatttggttttgggagataatttattttttttgttgttattttgtggtttttatagatttttttttttgttttttttttttttttatagatcgggattttaattttaattttattagtagttagttgtgtgattttagataggttgtttaattttttagattttagtttttttattttaaagatggaattgttggtttttgtttttaagagatggtgtaaggtttgttgagatagtgtaggcgatatacgtggttgggatttagattttggtgggtattttttgggttttatttgttttggttggttggtatttagagtatttggatttgtttttatttttttttatttttgtttttttgcgtttgtgagatttgatttattttttttattggataataagtaggttatagttcgggaggtggttattttattgtggtggtatatagtaaatgtttaagggttgttgattgaggttaggtttatatagtttattcggtatttacgtttttaggataattttgtagaggtttttaattttttgtttcgatattagttttaggaaatttttattgaattttaaaaatttatattgaaggttgggtatagtggtttatatttgtaattttagtattttgggaggttaaggtaggtagattattgaggttaggagtttaagattagtttggttaacgtggtgaaatttttatttttattaaaaatataaaattagttgggtgtggtggcgtacgtttgtagttttagttatttgggaggttaaggtaggagaattatttggatttgggagagggaggttgtagtgggttaagattgtattattgtagtttagtttacgtaatagagtaagattttgttttaaaaaaaaaaaattgatttagttttgtatattttttatatttttttattttattattttgtttgttttgttagtgaagttagtgatttttttatgtttttattttttagaagaggtagtattggatttagaatttagttttgagtcgggagttgttttatatggtattttaattgtttggttttgggatagtttaatagtcgttttatattttattttttttatttgtaaaatgggatcgtcgtggtgtttatttggttgggatttttggagattaggtgtgtgagcgtgtgtaagacgtcgagtgtagtttttggcgggcggatagttgatgatagtcgtggagttggcgtttttaggtcgtttttcgagtgtattttattattgtgttgggggttttttttttttttattgtgttttagtttttagatttttttttcgtagggtgttttagttttttcgttgggtttttataatagtttaggtttttttgagttgtgtttttgggggattttatttaggttttaggtttttgtgagttaggtatggttttagggggtggggtttttttagttagatatttatagttttgatattatggggaaggttaggtgtttttttatatgtgggattagaaggagatttatgggttgttatgtttagatttagtatgtttttttgttttggagagaagaggaggttgttggggtttttttggaggttttgtaaattatgaatgtttgtcgaggttatttttaggcgttttagcggggttttgttgtaatagggataaggaaggcgggggttttggaattagaggatgtgaatttcggggttttttttgtgttttttagtttttgggtaaattgttattttttttcggttttagttttattagtaaaatgggaataatagtttttattaatagtgttaggttagagtagttgttttacgtttgtaattttagtattttgggaggttta

SEQ ID NO: 114 - DNA, 1587 bp, Artificial Sequence
  Synthetic
ggatgggttgtagataagagtagttgagaattacgttttatttttttttttagaaattttgaaacgaggttttatttttattagaagtttagattgtattatggttattttaattttaaaatgttagatggtaagaatatttgtttattttttattttttttggaaaaagttgtttttcgggttttatatgcgattgtagttttttagtgtgtgaaattaggaaaataaaatatttaacggtgtatatttttatatttaaatagttagaaataataggtagttaggttaattatttttgattagtaagattagagttattagggtgtttattggtttaataaatgaatgtttttaggcgtttattatttgtaatttttagaagttttaatttttataagaaataaaataagaggggttttttgtttttaatagtgaaaagatcgtttttttttttttttttattcgggttaatttttttagtcgtttttttttgtattacgaatatacgttgtaggaaagcgtatttatagttcgggatatttttagattttttttttaaaattttttattttttgtgtataggagaaattgagagaagtttttattttttttttaaattttataagtaggggagggagttgtagtagatttttattttcgtttttaaaagcgaatgtgaaaaagttcgagaaggtacgttttgcgagtggaggttaaatcgaaatttgaatagaatgtacggttttcgtaaattacgattgataaagaagatattgagacgtttgcgggggatataagttatggttgtttcgttttttttttttttttgttaattatgttttttggagaaatcgtcggttcgatttacgtatatatttttgtaaaatacggataaaattataagtagttatttttattgtttcgtcggttacgagggaagttcgagttgagcggagggtagattttaagggtcgtagtttttggtcgtgtggatcgggtttgcggttgtagagcgcggtttcggttgtagtaagatttggggtagtgttcgaggcggcggcgagtatacgtggcgggttggattgtagatcggttttttcgcggcggagattcgcgatttagcggattgtattagtaggaagatattaaggttgtttttttaggtcgtttttagatggtggagttttttttagttcgaagattcggagttagcgtttagattcgagttttatttattgtttattttcggggtgtagggtagaggtgttagtgttgtaagtaaatgatacggttattttcgaattagttattgtgggtgcgtattcgagtgtggggatgttcgtgtaatatttatatggagacgttaaggaggaggaaataaatagattagaggttaaatgtgattgttatttcgttattattggtttttgtttatttttttattgtttttaaagtaattttgttgtatgttgagaggattacggtataattttgtttaaaagtatatatgtatttttcgcggttattttaaatgtatttttgtagtagttaagaatatgtgtttggtttgtcgattttttttttag

SEQ ID NO: 115 - DNA, 3381 bp, Artificial Sequence
  Synthetic
ggtaaattaatggattttttattattttttttttttttgggttgtagtagtgagttaattggtttagatttgtttagcggaatgttttttaggatatattgagtattgtagtaagagttgtatatgcgtttagggagaatgtggatagacgttgtttgtgatttacgggtaaagggtttttttttagaggtagtagaggtagattttgtttttggtgtgttttggttttcgatttggaggttttttgggttaggttaagatttttttcggtagcgatggtttttagttatatttaattgttttgaagggatattttttgtttatttttttgttcggttgtgttttttattcggaaggtttgtgtttttttcgtttgtatgttttattttgaggaggtttttttggttttttatcgttttttttatgggtttttacgttttttcgaattatcgcgtttaagtaggagtacgttttcggttttttttatagggcgtttttttttataggggtgttcgggatttttattttgtgtttttttggtggtttttataagtttggaagggtaggaggcgtattttatttttttgggtttttttttttagcgtttggcgggagtttaggttgtatttgtggaatttatgatttttttttttttgtttaagttgaatatattgttggtttttgttcgggtggagttcggttaattagagtgaggggtttttcgtagggcgaaggggtgcgttgttagatgtggtattttcgttttacggagatatacggtgttttatacgttagggagaggtttgagacgtaaagagtcgtcgagcgggttgcgggattgtttcgttgttattttcgtttgtagttattttttcgtacgtatttgtgtgtgtatttaggttaatatggaaggcgttattttaatttttgtcgtgagtaggtgggagggaagagagacgagaggtattttattggttgtttgggaaaatgaattgtattttttttttttttgcggaggattaattttttttattttttcgggtgggtattcgtattttggggtcggagtttgaattcgggagttaaggggttttagttttagggacgtgaagttgagcgtatagcgggcgtttttagatattggggaaagtgttttacgatgtttcgagtttttttagtttcgttagcggggcgagcgtgagggtgtttcgatcgattagcggtttcgggtgtagggtggcgggttcggcggcgcgcgttttttttttttttttggcggttcgtacgtgtcgttcgcgtcgcgtttttacgggttacgcgcgggtttcgtagcgtcgcggtcgagtcgggttgttcggttcgcggatatagcgtcggtcgtcgtatttcgtgcggggtcgcggcgcgatgttgcgttggaatgaggaagcgcggcggcgaggggagggttcgggcgcggtgcgcgcgggggtggcggcggcgcgtcgagcgggttcggcgcgggcgagcgggttgtagtcggcggcggcgttagtaggtacggttcgtattcgtcgtcgtttcggcggtttttgggggttgagtcggagttcggcgcgattgtaaagttttcgtgcgcggtttttttggttcggagttgcggttgagacgcgcgtcgcgcgagtcgggggattcggcgacggggcggggacgggacgacgtatttttttcgtgtttcgttttgcgtttttttgcgcgtttcgttttttgtatcggagtagcgattcgggaggcggtcgagaggtgcgcgcggggtcgagtcggttgcggggtaggtcgagtagggatcgttagcgtgcgttattttaaagtttgcggggtggtagggcgcgcgttttggttattcgtcgttttgggcggtagttggtggtaacgtaagggcgcggcgggggcggtcggcgcggagggggttaggtacggggttcgcgggcggcgttgtgcgcgcggggtagtcggtcggtcgggagcgcgaaagtttggtttgagtcggttgggggcggggagtgtggcggagaaatggggaataatgcgagtgagtaattttaggaagttattgtgaaagaaagttgggaagagtttcgcggttaagttagtaggatattttaataagtgattgcgcggttcgcgttcggggcggtgattgcggtaagttttttgggttttcgcgcggcgtattttagtttgggcgggacgttcggtcgcggcgaggcgggtaagtttggtagggtagagggagtttcggtttcgaggttgtttttcgtattcgaggattagttttggttttaaagcgcgacgtataaatttacgtgagtgtttttaaattgaattttaataggaaaatttggggtaattggtgaatttaaaaaaaaaaaaatatagtaaagaaaagcggtaaggttggtagattttggtgtcgtttaggttcgttttttttttttgaggatagtgagagagtttatttttgttaagcgtttgttgttttgtattgtgttagtaggtgtaggattaggtcgatatgggatatttttgagtagtttcgttgttattaggagaggagttttagtttttaattatatttaaatttatgtagatttatatatatttacggaagttagtttttataaagtcgtgtgtaaagagttttttttatatttgagtcgggagttttttttttatattataaatatgatgagatcgagtttgaatttaatttttgtaagagaggaattatttcggttttgaaaagttagtttttttgttgatcgtaggtttgacgtttaagttattaaatttttttaggaaaatttttagtaatattaaggtattaggttatttgcggttatatttgaaatgtattttaaatatttgttaagtatcgttgttgatgtttaaggaatttcgtgagggtttgtttttttttttaatttggaggtatttaatgatcgaaaatcgtagcgattttatagggtttgattaggtatagtttttaaatgtagtttttttttttttagggattgtagtttatttagattgaattttaatgcggtgcgttttgtttaggttatttatttataattttttattgcgtcgtaggtagtatattttagttttgagatattttgttttaaaattttagataaaatggtgttgaggaaatgtttttttattagttttattaatttttgttaaaagaggaaaatttatggaatttgaaaatattgcgtatgatatttaaatttttatagatatttaaatgtttttaaggttaggtttaatttggttatgagtcgaggggtgggggggatttatatagaaatgttttgggtttttttgagtttattt

SEQ ID NO: 116 - DNA, 7398 bp, Artificial Sequence
  Synthetic
ttggagtcggtgggttttgagtatagttacgaaggtatgcgtattttttggttgtttttttgttattgttttttgggtttttattttggtgaggcgtgtattttggcggcgttttttagggaattaaatatgtttgttgtattgtgcgtggagatggagaatgtataattggttgattttgtgttaatttggtggaattttatgttagttttgggaaagaataattgtatgggtgtgtttatatttattaggtgttttttagaaaaatattcgagaataatgttgtggtttaggatggttgttgtgtcggattcggtatttttttaggggggttgtgttgttgggttgagttttttaggtattggatttttaaatttttaaatacggcgtggataggtggtttagtaggggttggattattcgataggtttaggtgttggagtttagataagatatattttggtttggcgtggaagatacggggtgttattaatggtagtaatggttgtatttttgaaattcgggtttttaggtcgacgagggtgtgtacgtatttgaaatgtttgtggttttgtagtttttatgtttataaatttatttggttgaaaatagtttaaaatatttaaagtatgagggagggagtgtttgttttttttaaaaaggaaggatttgattttatttatttaaaaagttatttaaatttagaatatttttcgtaagagattttttgtttttcgtttttttagaatggttggagagttttagtatttttgtatatttgggatattttagagggggtggggaggggtaagtgggtagcgagcgattttagatttaggatgagttgttaggcgtttttcggttatatatttaagggatcggagtgtagttgtagcgttgcggtttgttgtttcgggggtgggggtgttgttttatgttgtgaatttttatatggtttttgattttgggtagaggtcgagggtttaagggacggggtgatagggagagtatgtaggagtgggtttttggttttttagggcgagtggaagaagcgttttttttttttgtaggtgatagatttggggggttttttttgaggatgagagtttgttgttttttaagttttgtgtttaatttaggtttttaggtttattttagtttttcggttttgtttgttttgtggatgatatagtttaagggtagagatcgttggtttggagggaaggttaggttttaggttagggtttagaagggagggagaagtttttggggtagttttttttttgtttatttattgtttagtttttttttttatattttttttcggaaacgtttgtttttgataaggtttattttttgtttttaggaggtttttattgtggaggaagggaggcgtcgttcgtttttggtttttttgatagtcgtgttttattttcgttttgtgttttttttttcggatagtgttttttttagggtttatttaggagggtgtagcggtggttttcggggcggtggtcgtggtgggggtgttagttgtaggggtgttttcggtgggtgggagttggtggtttttcgttggtgttatgggattcgtatgttcgttttgcgtttttcggtttttgagtttataggtcgggattttgtttgttagtcgcgtgcgttgtcgtttaatttttgtaggcgtagagcgcgcggcggcggtgatagagaattttgtttggttgtttaaatatagttttttgtagaaggattttgcgttcggggaaggggaggaatttttttttttttgggcgttcgtttttttcgttatggttcggtttttatattcgtttatatttggtcgtagcggggcgttcggggggaggggttgaggtcgcgtttttcgtcgttttttgggcgcgggttaggcggggaggaggggggcgtttcggtcgtgtgtttaggattgttttttagcggttattcgggttttagttttttaggtttggttttgataggcgggcggagtagttagtgcgagatagggaggtcggtgcgggtgcgggaatttgattcgttcgggaggcgggggcggggcgggggcgtagcgcgcggggaggggtcggcgttcgttttttttttttatttatttagttgagttagggggtttaggggttttttcggcggttagttttgtattgtaggagcgcgggcgcggcgttttagttagcgcgtagggttcgggtttcgtcgggggcgttttttcgtcgttgtttttcgcgcgattcgttgtttattagttattatgtcggatttcgcggttaacgcgtagttggatgggattatttcggatttcgaaggtgggtgttgggttggttgttgcggtcgcggacgtgttggagaggattttgcgggtgggtttggcgcgggacgggggtgcgttgaggggagacgggagtgcgttgaggggagacgggatttttaatttaggcgttttttcgttgagagcgtcgcgcgttttcggtttcgtgttcgcgtcgtttacgtgggggattttgttaggggtattcgcgtagattttgcgcgtttttataggattttgtgttcgttttgcgtattgtcgtttgggtttttttttttttattgttgtttgtgtttgttaagcgatagcgattttttcgagggttcgcgaggttgtttcggaattttttaggacgtatagttttattttgggaaatttatcggttttttttttttggtttttttcggcggttttcgggtttcgtttggattcggtaacgggatagggaggtcgttttttattttcgattgagtggatagtcgcgttttgttcgggtggatagttttttttttttttacgttagtttcggggtcgttaagttgtgtagttcgtgggtcgggagtatcgaacggatatagtttaggtcgtggtagggtttagagtgggatgttttatggtttttatttaggtttggggatatttttattcgttttttagaatcgggtcgtgggggatagaaggggtttgcgtgcgggtagggagagtattttggtttttttttgttttcggggtttataaagtgtgttgggatttgcggggttgttttgtttaagtttgggtttggcgttcgcgtttttgagtttgtgagtgcgtgcgtttttttgcgtttttttgattgtcggtgttggggttttgcgttttgcgttcgcgggagtaaatatagtaggcgaaggggaagtttatataatggtttttagcgttttggggtagggtttttgaggggcgggtttgtttttgtcgggatttggagttttcgtttttcggagaggtttttaggttgatttgggtagagttttttggtgggtcgggagggggaaaggttgtgttgaaatgagtaaattgtttaggtgttaggttaagttgggaggtgattagtttgaggtttttttcgttttatggttagaattagggttgatatttgggtgttttgagtttagttgtttatacggtttatttggggttagttttatttgagtgggggaggcggggttttttgggggattagaattttggttggacgttaagtagagtgttagtggttgttttttagggttgggtttgaggagggtgtggggcggcgaagggacgggagggggttgtgatttagtggttattggcgttgtgtagagtgtgagttggaaatatcgtagttattttgttagtttagtggtgaaagtttttttttaggttttatttttttgtatttttgttttttagagggaggggaggtttgggtttgtagagttgggagggtttgttgttttcgttttttttttttataatatttttttatttggatatttttgggtatatgtttatattggggtttttttaggtttattgtgtttcgttgagttttttgtagttttcgagtgaatgtgatttttttgtttttgtttttttgtaatttttttttgcgatcgtttttttaggggttttttttgttttaaatgtttaagtggtacgatttagtcggtttgattattttttagtaagtttttatggagagaggttttgtgttgtgtagagttttttttttgtttgcgggatcgaggtttttgtttttagtttttaatagaaagtgtcgggtttttagtgggatttttggggaagaattttcgtgttttaacgggagttttgtggcgggaggggaggttagggtttggggttgtgttcgttgtatagttgttattatttgtattatgaaagttgttagtgttttttttttgggtttttgggtgtaattttatttttgtttttatgtgtttttatttggagttgtttttgcggttgttttttaagttagttttgtgattttgtaatttagtttaagataatgggtttattgagattattttggtgtagtagttggtaattttttggttttgggggaaggttttttagtttcggggagtggggttttaatttgttggttttttgtgtttattagttttttttttgtgtgttttgaatggttttgttgggaattttggttttagagttattaggtggttcgagtcgataggcgtgagagagtgtgtgtgtgtatgagtgcgtatgtgtatgggggttgatttggggtatggaaaggtggttttttttggtgtttaaggagtttggagtatagttggagggtgtgggggtgtgtatatgggagttggataattttgggtggatagatagacgtggggaagggatgattgaaggaggtggaggagagagtgtgatttagtttagttaggggtgatgtggataggtagttttcgaattagggtagagaaaagttattattagttagtaggggagaagttagtatggaggaggcggattttgagggagagtaggaattggattgtaagaggaaggagagttttttggttagtagtagttagtagtagtgggggaggttggaatgagttggttggagagggggttggggtataaggaggggtttgtttgtgaagattatatgggttaggttgcggagggttaggtatgttcgtcgggagtgtagttggtttacgggaagtatttggagtggttgggaatgggcgtaggagtagcgtcgtgggagtataggtttttttttcggggcggtttatttggtgttttggtttttgtaaggtaggtcgaaagggtggggaggaaattgttagttttttatagcgttgggatggtggttttagggtttttgaggttagcggatgtgggtgtttgttattatgtgggttgttgaggggcggagattttaggggttattttaaagtaggacgagttttgagttacggtatttttgggggtagttttttaatcgagtagacgtttaggtttggaattttgtaatagaggttatagggttttgattagggtgttttgggaggtttagaattagtggtagtatatagggtagacggtaagtgatttggtatggggaaagaggtaggtgtttaggtcggtatagtatattcgtaaggaataggtagacgggaagtcgttcgtgggtttgtgtgtgtgttcggagttaaaattttgttaatgttttatgttttgggtatatttattttttttttggggagtatttttttttttatttttttttttttcgtttgttttttttatttagggttttttttatttttttcgttttggggatcgagggtattatggttttatgttttattatcgatgagttgtatagggatttagttttttcgttgtttaggtcgggttttttaggtttagggtttttaggaatggagagggtattagtgttttttatggatttaaatttttcgtatttcgtttttgttttttttttaagataggttttcgagttttaaggttttagggttttgtggaggtcgttacgtagtagtaaggagaatgttttgtatttggttgatgagatttttagagttttattttttatttttttattgtataaacgggtttttaggcgattgtagtattcgttattgttcgtaatagggtgataagagggatgattttttttttttttttttttttggttggtggaggtacggggttggcggacggtatgtgttttcgtgaatttaggttaaatttgttatcgtaaatacgattataattcgggtttttgtgtaataaaagtttttttaagtattagttgttggtttgttttgtttagcggtgtttgttgtaattagatttgtatatcgagaaagaatttaaaagtttttgatgtttgttgaaataatttggtttaggatttacgtgtttagattttagagttgtgtggtatttgagtttttttcgagtttttattgtcgttcgaggaggatttttagatttgtgttttggaggtagagtaggttgtgggacgggtttttgggtgggaaggattatgtggatatgtttttttgtttgagagttttaatattttcgggacgtgggagttggcgcgttggtaggatttaggtgttttttttttttttagagaaaaaggtttcgttgtttggtaataggtgtagatttgtttttaattaatgttagtaggttttttgcgtgatgaattttgttttttagttaagatttaaggtattttgtgaatattgtttttttgtagtttgagtttttgtggtgggaggtaggagttatggggagtgggggtaggttttttatacgggttttatagttattggtagtattgatttgatgttttttgagtttagagtttagggttagatagatttattgtttcgattacgagttggtttatttagaggggggcggatatagtatttaggtagtagatgtattgtgattagttttgtagcggggttgtgggttttttgggttggatgttcgggaagaggtaggtggaggtaaacgttaggatatttttgtagtgattgggtgattgtaggttggaaatgttttttgtgggttgtggttgtttaggaaggttttgaatggggttagtggatagagtttgtatttagaggggtagtgttttggaggagtgaggggtatggtagtgtagggatgtttaggtcgtttttattttgttattggaaagttgggcggtttcggtttttttagttttttcgtttgtttttttgtttgtaaagtggggttagaaatagttttttttgagggttgttgggggattttgagatgtagtttatggcgttgagtacgggttttgttttttacgggtgtggtgggtgtcgcggttggtgtggtatttgggcgggaaaagggggtatttgtaaaggataggtaggtttggatgtttaaatatgtagatttggggatgggaggttttaggtaagggtttgtgtgatgttattgta

SEQ ID NO: 117 - DNA, 2396 bp, Artificial Sequence
  Synthetic
gaatattttatattgtttgaaagtattttatatttttttttttttttaatttataaagtagtttttttttattggtcgaatttttaaggtagaaaagttatatacgtttttcgtttttttattaattgttttttagaaaagggaaagtgaagaagggaaagagaaaagataacggggaagaaaagagtatagaggagagaggaaaagtggggagagaaaggaagaaaaggattgagaaaacgtaggagttttggtttgtcggtgagtagagtcggcgtagttatagcgcggagtcgcggcgtttattggttttcggagttgttaatcggcgtgtaattttgtaggaatttttttcgggtttatttgggagttatattgtcgttttttttttttagtcgtttaggggagttcggagaagtaggtttaggagggagggagttagaggaaaagaagaggaggagaaggaggaggattcggggagggaggcgcggcgcgggaggaggaggggcgtagtcgcggagttagtggtttcgtttggacgcgttgttttttagatattttcggagttttagtcgcgcggatcgcgcgttttcgtcgttttgtttttaaatttttgtcgtagtttttttttaagttagcgaatttattttttaaaattagaaattgaatttcggtacgggaaaggagttcgcggaggagtaaaattatagtagagtaagaagagttttagagagtagttttttcggagtattaatttcgtgtcgggagtgtagaaattaataagtgagagggcgtcgcgttttcggggcgtagttgcgggcggcgggagtaggcgtaggaggaggaagcgagcgttttcgagtttcgagttcgagttttcgagtttgagtcgtaatcgttgcggtattttgtttcggattcgtgtgcgcgggttgcgtcgagcgttgggtaggaggtttcgttttgttttggttgtaagtagcggttgggagtagtcggtttttggggaatatgcggcgcgcgtggattttgtttattttgggtttggtggtttgcgtgtcggcggagtcggtgagtgggttaggcggaggatgcgcgcgtcgtttagggtgtttgaagttacgagaggagttcgtagggaataggggagcgttatttggggaatttttagtttttaagtatatatcggagattcgttgggataaatgcgttcgttcggttattttttttttttttttttttttttagaaaagcgttgttcgttggcgttatttcgcggttcgcgggaatgggggtatcgagaattgcggtttggtttagtcgtagaggtttttgaagttatttttaattttttcgttttcggcgggttttgttgcgtggtttgggaaggacggaggggaaagggtggtaggaggggggagtttgggtcgggttcgcgagggaacggttttatttcgcgcgtttttcgagattagggatgatttggaaatttcggggttttttttttcgtatattatttttttcgcgttagtttttttgtttgattgtatgtaagttttggggagatgggggttagatttaagagattcgcgagtgtttagagagaaaagtttgtaaaagtttttttgtttgatgttttttgcggttagggcgaggtaatcgatattacgtggaatcgtagtaggcgattttttaaggggatattgggggaggtacggaacgcgttcgaaaatgttgggacgtcggttattggatttttagttttgcggcgatttttttttcgttgaggggtggaggttgtatcgcggggcgttagggacgggaggatatttttataggagttatacgggagtgtcgtaagtagggcgaggcggggtacgtgtgatacggcgttcggtttcgggtcgtttggtcgttgggggatagaggtttttttttcgttacgttcgttttttttggttttggcggggcgtttttggggtcgggaggagtttcgttttcggcggagcgtttgtcggtatttagtttttttttttcgttttggcggtgggaatttgatttttttttttggtcgcgtttcgggggttggagtttgtttttttacgtcgtttaatgagcgttttttaaagggaattgtttttttggttttttttcgttcgtagttgtttttatttgggcgttaggagttttgtcgggttaggtggaagtttgagtattttagatttcgtttgtagttttagtgttttttggggttttagggagtgcggttgtttttggtttttttggttttttacgttttttttttttgttatttaataacgtgttaatttttaattaattgaatggtttttttaaagataaattatattattttaaggttttttaggtagttttat

SEQ ID NO: 118 - DNA, 3696 bp, Artificial Sequence
  Synthetic
tgaatcgagtttaatatttttttgtataaaaatttttcgataggataaataaattatttttttattgaattgtgatttttgagttgattttagggtgtatttaattttttagaggtgtttggtttaatttttaaagaggtagttttagttttaggagtcgatcggtattatagtatttgagtagggtattgtaggaaggaaagcggaatttttagtgaaattttttattttttgtcgttgttgagagaggttggattcgtttttttttatatggtttaggaagagttgtaaacgggagtttgtcggttggttattagtttaggggttttgggggcggttttcgggttttgttttttgtttttttttttgattaatgttatttttggatagataacgggattaaacgttttttattttttcgagtttttatgtatgttatggttttttttttaggaaaatatgagtataagacgtaaagtaaaagaagagattaaaggtaaattattttattatttcgtttcgtttttttttttcgttattatagaagagtttgattaaattttttttgtcgcgttatatatacgtagagtaggtgaagggtattttaagataatttatttttttttcgttttatagaaagtttttacgaaatttttatattattttcggacgtaaagttttcgtatttagtttgaggagttaaattattttaagttaaggtaggaaacgttaaagtggtgtcgaagtggttttaaagtagaaggttgggaagtagggtaagtttagcgtatttagacgtttgtatttatataaagaaattagtcgtatgattaatgggagttgtcggttggaggcggggcgttcgtgtcggtttttttatttgggatatttgcggttttaggttgattttttttaggttgatggagtgcgttcggaggttgttaagtagttggtagagtaggattttatcgcggagggtttgcgtaaggtcgaatatttgagtcgagttttaggttattcggtggttggtgggtagtattttgtaatggatgagttattgcgcgtattgtttttacggttttatgttcgacggtttcgggacgcggttgggtcggggcgggcggtaaggatgcggtcgtcgtcgtcgtcgtcgcggttttttcgcgtttcgtcgacgttaatagtcgtcggtttttttttcgcgcgggatcgagggagtaggagtcgcggttgacgggtcgcgggcgtcgcgttaggttcggtttcggtttcggttcgggtgcgtcgcgattcggtcgtcgttgtagcgagtttcgcgcgtttttcgtgcgtttcggtcggttcggcggcggttgtcgcgtataggttttcgattttagcgttcggttcgttattgagtatgtttagtacgtcggtcggtttcgttgcggttcgtaagtttttagacgcgcgggtgggagcgcgtcggcggtcggggttggggtttgtggtcgagggcggggcgcgggggaggggtcggcggaggggggcggcggttagaaaggggatttcgcgttttcgtttgtagttttgcggggtttacgtagttttcggcgttttggggttttttttcggggcggtttttcggttttgcggggagtgtggttgaatattgtaatacgatggggttttttaggattttttaaataataaatgtttaaaggagcgacggattaattggggtatttagattttttagagtaatgaaaaaagtgtttagagtattagtagagtacggcgttgaaagttttagagatcgtcgttttttcgttttttgtagttttattttttttattttttatagttgattttttagtttattttttttagttgtttttagtggtgttttggtatttttaaagtgagtgagagtgcgtgtgtggagacgtttgcggaaatcgtttcgatttttgagtttattttttcgcgaggtgatttttatttggagttggtttgtttttgtattgttaggttcggaattgtttgtcgttgttgttttggtttttttgttgattttataaaaatttgtttgagaaaggtttgtgttacggtgttagattgcgtatgcgtcggcgattggcggtcgggtttgagagtaaagcgcgttagttttgggtagtttttgtcgggttgttttgggatttttagtgaaagttggaatttgattttagaatttttgcgtagtgtataagtagtgtattcgtgttttttaagtaaattttttggagattttaaattttattttttttatttttattgtatattgttttttaaaattataattaatgtttaaattatttgtggtaagatattgttatagtgaattattgttgttcgataggaatgtggtttttcgttatttttttgagatgtatttatttaaaattagaaaataggtcgggcgcggtggtttacgtttgtaattttagtattttgggaggtcgaggcgggcggattacgaggttaggagatcgagattattttggttaatacggtgaaaattcgtttttattaaaaatataaaaaattggtcgggcgtggtggtacgtatttgtagttttagttgttcgggaggttgaggtaggagaatcgtttgaatttatgaggcggaggttgcggtgagttgagatttagttattgtattttagtttggacgatagagttagattttgttttaaaaaataaataaataaataaataaaaagatgggagtttggataatttaattgtattatatttttgtagtttaaattttttattacgaattatgttttataagtaggttaaaatattatttttaaaagaatgatttgtttaggatagtgattgcgttttttttcgtttggttgtgataaggatagtatattgtttatatttagcgttgaataataaattagtttgaggattttttttttttatacgtatttttttttagtaatttttttttgtgtttaatgcggattttattaatagtacgatattttttaggttataattttggtaaataaattatattaatttttatatatttttagaagtaaaatttaatatatttgaattattatttaatatagttttttataaagagaaattaaaaagaaattttattaaacgggttaaaatttaaattaaaattatgaattttttttaggtagtattagattatttttggatgttggagaatatttttaaaatttatattatatatattattttaggacgtaaatttggatttagagttcgagttgtattgttttttagtaatatgattttggataaattttttatagtttagtttttatagatacgaaataagaaatttttttttttttttgtggggggtggacggagtttcgttttgtcgtttaggttggagtgtagtggcgcgattttagtttattgtaagttttatttttcgggtttacgttatttttttattttagttttttaagtagttaggattataggcgtcggtcgttacgttcggttaattttttttgtatttttagtatagatagggttttatcgtgttagttagaatggtttagattttttgatttcgtgattcgttcgtttcggttttttaaaatgttaggattataggcgttagtcgtcgtgtttggttataagaagtaatttttaattaatagggttgtt

SEQ ID NO: 119 - DNA, 4199 bp, Artificial Sequence
  Synthetic
tgttaaaattagaatagattatatttaggggataatatttaggtatgttaacggagtttaaaatgttaaggaaattatattataattttgtttagtatattataggttgttaaatcgaaatgttatgttagttaggagtgtagtaatttttattttttggttttatttaattaggaagttttagtagagcgaagtttgttaagcgttcgtcgttagaatttgaaggaattcgagcgagtaagaagagtgtttgatttattttatagaagtttgtttagaaatggaggagttagcgtttattgaagtcggtttcgttttcggttcgtttatatggagtttgattagttttagttatgtttatttcggtttgggagattcgtaaagtgtttttttttttaatttttttgtattattttgaagtttagggaagtaaagagaggggtatatttggattgtaaaattaatgttttttgtcgtttaggagagaagggaatgagagagagagagagatagatagatagagagagagagagagagagagagagagagagagagagagagagagaaattttattgaaatttagtttttttagaatttgtgtgatttggtttttaacgggagattagtgcgattttatggtatttttgttaggaattagcgatttttttgtagttattatttgatttattgttttttcgtttatttttttttataaagttattttttttttattttagtaagatttttttttttaatgatgataaagtttttgttttagtgttttttttaggattggtgtttttttaaaatagtgaatttagaaaattatttcgtttaatattttttaaaattttcgtagttttaatgtaagcgtaagtatgtaaaggttttttgttatatttgtattttttgtttattttagaattattttttattttcgggtttgtaatagttttttttgtttttttggatagaggtgggtggtattaggggtttagggtagtaggaggtgaggggttgaggaggcgcgttagggtaggttggtttgtgttggatacgcgtgtttttttgcggagttaaagggtcggggacgggggttttggatttattagagtaattttagtcggtgggcgtttggtagttatttaaggaggtagggaaagtagcgagttttatcgggcgggttacgatgagtagtatgacgggtagtagtagtagttagtaaaagttttcgtaaagtgtttagttgttgtattgtcgcggggatttttatagtattatgattagttcgtgtaattttgtagtagtaaacggttttcgaggaatataggatcgcgggggtcgggtagcgggttattgagtatttcgcggacggcggtagtagaggcggcggcggtggtagtggtattcggcggggaagtagtagttaaattcgcgtatgatttcgagagttttagtaatatttagggattgggtttagtttcggagcgagagggtcgttcgttgagaagttgcgtcggagacgcgggaagttgttgttataaggagggagttttgggaagtcggaggataggaggagacgggagtttaggggtagacgagtggagttcgaggaggtagggtggagggagagttaaggcgtttcgtagttcggtagtcgtttttcgagttttgtcgttcgtatttttttggcgtttgggaagtagtaggtttttagttcgttcggggttacgtgggaagaggtagtcgggttttgattggtggagtaggatgtaggtttcgggagggaggggtcgacgaggaggtgtaaggatgtaaggaggaggcggtcgcggaagttatagatgggttcgttcgttaggcgttggttcgagtggggttaggcggggatggtttaaatgagaagttcgggttttagggtgggttattcgtatatttatatattattcgttttatttttcgttttaggacgttttttatcgaaggcggggttcggattagcgtttttttttcgcgcgtgatttcgggtcgcgagtgcgggtcgcggttgggtggcgttttttcgagttggagatggtgggggcggaggtgttagaggagtagtagtagtagggtagagaggggcgagtcggcgcgggagagggcgttttgttggcgatcggcgttttagcgtgcgggagcgcgtcgtttaggttgtagggggatgtaggttgggaatgtcgcggcggagaggttagggacgtttttttagggatttataggaaagagggtgagaggcgatggtgttagaatcgtttttgtcgatttggaagtaatagtagtattttttataagagcgtgtaattttaaggttgttcgtcgaggtagtttagttatttcggtaggcgttttttttttttttttttttttttttttttttttttaggtttttcgtagtttcgatttagtttaagcgttcgtaggtttgaatttttttttttattattcgtttttttttagttcgtagtttattagtgtgtttatttgggaggtgcggttagatgtgtttggaaggttagattggtcgggataagtggtttgagagaaagagaaaggtttttttgtatacgtcgcgggtgggttgtcgggagtatcggtcgggtagcggcgttcgggaaggggagagcgggttttatttgttggtttaggtagtgattttgcgttttttattcgggtttttgtcggatggtcggtgatttggggcgacgagagaaggtttaattcggtaggagtttttggttttgcgcgttttttttattttttttagcgggaagggtaaacggtatagcgggattcgtttttcgtttgttgtattttttaggtagttagatatattttttagtttaatggaattttagtcgttagtaacgggattaagagttttcggggataagggtggagaggaatattttttttttatgatcggggttattattgtagttttagtgttttggatgttttatagggaagagttttttttttggtgtgtgattatttagtgatttttgtttttgtttttgtttatttttttttcgttttttttttttatttttttttgttattttttttttttttttttttttcgtttttaaaagttttcggattttttttttttttatttaaattttttttttgtgtttttttttttgtgttttttgaatttaggagagtatttgataatatttaataggtaattagtgtttatttttaattatttaaaagaggtatttatatattttgaaaacgggattatttattttttgtagatattagtagaaaaataaattgtattcgagtaatttttttaagtattttaatttttaatttttttttatttttttgttttttaattttttttttgagagatgtgatcgtgtagtattttagtgttttaacgaaattttttttttttttttgtgtgaaatttatttttttattttatattttcgttttcgttcgagattgtttttttttttttttatttttaaagatttttgaattttagtgttttttatttttggtaattaagtagtagattttagtattttagtcggtggtatttcgttttttatcgacgaagattttattaaaatagattaattagattagacgttggaggtattagaaaatcggtttttagatagagtagttaaattttttaaggaaatagaatatttattagatagagttgttaattaatattgtaaaataaggaattagaaatttttttcgttataggtttttagtagagaaggtaatataaatatagattaagatttaataattttatagtagagaatgagaatatgttattttttatagtaaggttggtgtggtaattaattaggtttatgaaaataagttatgtttgaaattaaaggtaaagtttttaaaagtgtttatgtagtaattatgataatgaaataggatttgttaggattttagagtttggttatgtaagtagaattttagagaattttttagtagaggaaaattgtttttgaattttttgttaagtaaatttttggtatattttttaataatatatgttttttttaagacgttttgttaaaagtaagttaaaattttaaaggagttaattattggttgtaattggttaataaatgcggttgtttttatagaggttttttaaattatt

SEQ ID NO: 120 - DNA, 5716 bp, Artificial Sequence
  Synthetic
tgtgtgtgtgtttacggcgcgggtcggagtatttattatttcgtcgggggagcgaggttatttcggggtcggattggaaatgttgaggtttcgtttgtttttttcgcgatatgttggtttaaatattagttggggtagaataaaaaattattaaaaaaaaatttttttaaaattacggaaatcgagcggcggcggcggcggcggtttttttcgttcgtcggttcgttcgtttttttttcggtttttcggtttcgggcgtagcgcgtatgtgttttgttataattatgattattaataatgtattgcgattaattatagaggggttttttgaaaggcgattggtatcgggttagcgttatttaaattcgttcgttttaattaattagttcgtgatttgttgtagatttttgttttttcgcgcgttggtttaataagtcggtcgcggggttggttttgcgcgtcgcgtcgttcgatattgggttaattttttttgcgatcgttcgggatttcgcggttcggtcgtcgggggtcggttttttttttattgcgggttcggcgtttcggtaggcgtttttcgttttttttttttttttttttttttttttttttttttcgtttttttttataatttcgggttgggtcgatttaaattagtatgtttttttcggaattgagtcgtttcgggtggggggtggggtcgggcggcgagggttggggattcgggttgtcggcgcgttgcgaattttttaattcgggggcgaggcgcgtcggttcgcggggggagggagtttcgcggcgggattaggggggagggcgtttcgtttcgtgagtgttttttcgtttttttaagagttattagaaagtagtttatttttatttttattaaaaaaaaaaataagcgagtggggaagagttagttttacgtcggttcgtttttcgtttacggttcggtttttttttcgtttggttcggcgttttttcgtttcgagttttttattttttggatttcgggttcgcgggggcgcggagaagaggatttaggaaaggtttgggaatttgatgggtttgtggttggggggaggggtagttttttcgttattttatttttttttcgttaggggtattgattttttagttttttgtttgttttttcgttttttttttttatttcggatatttttaggataattagggttgaagttttcgggagaagtagtttcgagttcgtgggataagggggcggttcggtttcgtttaggatttttttcggatttttttttcggttgtttcggttggttgtagggcgcgttatttcgcggggaggcggcggtagcggtggcgtcgtagagtttcgggcgggcggagagcggggatcggtttgggtttcgagtttagtttcggtcggggggaggggaggggttgcggtttatattgagcgtagtagcgagtatcggtttagggagatatagttcgggcggattcggtattggattgggttagtttttgggatttttgggtatttcgttttggggagggggtcgcgttttagtttttcgggacgaggtggcggcgtagagtcggaaagcggggtggggggagggcggtggggaggtgttttgttcgaggttgtttggcggcgtcgaagttcgtttttattttttgggtatttgtcgagcgcggcgttcgaatttttcgttttattattattttttttaagttgagatgtaatttttttattttcgttttagagtttagagttaggagttcgatttttttgtcgggtttaggcgttcgcgaatttgagattcggtacgcggtttagtttagtttttttttttcgcgtttcggggttttttcgttttaaggttttcgaagttttgcgttcgaaggattaaattaaattaaatttcggttgagtaatgggggaggaggggggtgtagatatataatttttgttttattttacgattaggaaaggagagggtttataaggacgttttaattcgcgagcgtttttgagtttggtcggtgttacggtcggttcgcgcgaagtgggtatcggtttttcgagtcgatttcgtgtatttcggatttgtattgcgtttcggtagaggattttttttttttttttagttttttaaatttttttttttaaggtgtaaatgtgtgtgtgtgtcgatgaatgtttttgttttttatttgttgattttatttttttttattggtataattttttttttttttttttttgttcgttcgttttttatttttttttttatttattttttgttttcgtttattttttaatatatatatttatatttttttcgtttcgttttttcgttttcgttaggttttgatgggtatagtagattttgataaaaagataaacgaagttattagtggggttgatgttgaagaatgaagataataatgtttttataggtggtgttttaaatgttattattttttattgaatatttaaagagatttttcggtaaagatggatttgcgtatttttggggtgtgagcggttcgtattttttagatttcggggaacgtgtacgggagtacgtgtaaatttcgtattcgtttttattttataaataggggttttcgtatatttattaatttatattgttttaggtatacgaacgcgtttgtgtgtatttgtatatttttttttcggaatgtgttcgtgtttggagtcgttcgtatttttttttaataattttggttttttgaaagttcgggtttttcgggtatcgtatgtagttaggaatttagcggagttggcggtttcgtaaagttttttttttggtgcgcgtcgcgtgcgtagtaggaagttttcgggaataattttatacgtgtttgttatttggtttacgtgtgatttaagattaagtgtaggaaaaaaaaaaattttttttttttaaaaaaatattataataaataattaaacgaaatgaatatgcgaaaagtagttacgcgatgtttaattaaaaatgggcgagtagagacgaataaattgggggatttaaattttaataatttttttcgtttttttgcgttttcgacggcgtagaggcgggcgtttcgatttgcgtttggcgtttttcgttaattttttgtttgttttttttttttcggtggtttcgggaaaggagatgggaggtggaggagggggggatgggaggagggggatggtcggaatggcggggagaggagaattggtttttattgttgatggtaatatattaattacgggttattgtttcggttttaagtttcgtggttcgttggtgcgggcgttgtagtgttagggcgttggcgaggtttcgcgtgtcgcgatgtaaagaaatatattaataaaaatagaagtagagtgggggttattttttatttttgtgcgggtcgaaagtaggcggcgagcgggagtgaggtgttatttttggagtttttttacgcgttttgtttattttagagatttgcgcgttttacgtgcggtagtatttttttaattttgggtggaggtggggtgggggggggattattggggaattcgggtagcgaagtttggatttgtaggtttcgttagagggtttggtatttaggggttgattcgtaatagcgaatggggatagttcgaagtgaggcggtagttgcgtcggggtcgcgattttattggcggagaggaatcgtattcgggggaggcgtttggcgagttttgtaaatttttattttgggggattgatgttcgtttttattaatacgtttttttagtttttttaagttttagagtatttttggtgggttggcggggtcgaaggtttgtgttttaaggtcgtaggttataattggggttttagttatgaggtttttaggtttttttgtttaatttttggttttacgaagggtaacgtggggggcgttaaggttcgcgtattttgaggtttggtttttttatttttaaaagtattttattttggttcgatttgatttcgggtttgggatggggtttgggatgaggttttgtttgggttttagattttttttttcgtttcgttgacggtgtttggttattttattttagaaatggaggacgtttttaagtgttttttttgtatttttgtttttttagtttttagtttaaggtttcgttggttttcggtcgaggttagaagggtagtagtggaggtttcgtttttttaagtaaggtaaaaagcggcgaaaaggcggtagcggcgtttcgggggcgggggacgaggtatatcgatggtattaatattattgatggggaaaagttcggggtaggagttcggagattaggtttttagttttttttttgttcggaggtgggtgggatagagcgggtggttagttgtgtgtgtgtgtgtatgtgtgtgtgttggtgggggggtgtttttgaatgtattcgtattaattatttggggtttatttgtattttttttgtttgaaagtttgcgggggtagagggaaaaggaagggttgaggtggtttgtattttttagtttttagggtttttaattagggttggggttggggttggagttagggttgggggaggggtgcggggagaataggaaaaaaatgatagaaaaaaagattaagtttggaaagtttttcgtagattcggttatttttttaaatttcgggattttgttttttcgggagttttagaaattttttttttattgaattgttgttatttaattttaattttttttagaatattggagcggtttcgaattcgaagtatggagtaggtttgggtgggtatttcgtttttggttaagtttatattgtgcgttttcggcgtattcgttttttagtagtttaatattagtagtaaaacgtaggtttttgggggaattttcgtcgttcgttattaagggttatttttagacgggcgtcgggtgtagcgtcgtgatcgggcgttttggcgtcggttcgggcgcgaaatttagcggtggtaagcggagggtgggtttggtaattattcgcgcgcgttcgagttaagagtcgcgtattgtttgttcgcggtaaagttcgttttttttcgtttggagggttgtttttatatcggtattaagaaatcgatttcgttagcgattgtaagtgtttgcgattttgatttttcgtttatagttgagcgttttgtatttaaatttattgcgtttcgtatgtaatagtgtttcgttaaggggtttttgggtatgaaattttttttagaggaaatgtttcgacgtagagaaagtggaaaaggttaatcgaatttataaaaagaaaattttttttttttttggttaaatttttttgcggttttttgagcgcgaaaaaaaaattattattatgtaatagagtgtagttaatttttagaatttagtagtaggatgtttaggaagtttagtaggacgtatagatttttatttgtcgttttagtaggattttagttaggatttagcgaatagtggttagtttttgtgttttttgttggaaataggtgtgagatttgtggagttttgttttcggagcgtgtgtatttggaagagattggtagtttgttaggaaatttatttttatttataaatgagatttttttaaacgatttttttatggtatttttggatgaattaatttagtatattttttgtaataaagttttatttaaaaaaaatttacgttgttatttttt

SEQ ID NO: 121 - DNA, 5474 bp, Artificial Sequence
  Synthetic
tttttaggagtaaagtttttattttttttttatttattttttatagtttaattgattttttttttttttttttttgaataacgagaagattttttagttgtttaatttttatttttttagttgtaatatcgttattttttagtttagcgaacgttgggcgaacgtttttttttcgttttttttgttttttttttttattttaagttttcggttttttattcgttgaacgatgttttatttcgttcgtttttgttttcgtcgttgttgtcggagtcgaagtagagaaggtagcgggtttcgtgatcgtttcgagagtttcgcgttttcgattagggggcgggggcggtttcggggagggcggggtaggggcggggggaagaaagggggttttgtgttgcgtcgggagggtcggcgttttttttcgaatgttttgcggttttagtttttttttacgttcgcgtagttttcgtcgtagttttagttgtagttgtaggattgagtcgtgtattcggaggagattttcggaggaggcgataaatttcgtagtgtcgcgatttaattttagttttgggtaggtgagtgttttcgtagtttcgtcgttcgtcgtggggtcggggatagggagaagggagtgtttgtttggtttgcgtttttcgtttgttagtttttgtttcgaggttttggggtatttaattcgtcgatttttgatatcgtagcggggtaggttgttggatagtttcgagcgtttgtagttgttgttgttatttttgatttatatgtttttagttttgttagtggtagtttttttgttgttcgtagtttgattagtaatttttcgggttttcgtattttttttgttgcgcgtttttgttttagcgcggtcgtcgagattttcgattttgttttaggtagggcggtagcgtttcggattagtttttgttttcgtgtttttacgtataggtagttttaggagtagcggttagtaatttttttggggatattttattgtagttcggtaggatagtcgattagagtcgttttagggggtggtcggagtgtttatttcggttggaattttaattcggttttttgttttgtgattttggggtagttagttgatttttttgaatttcgggttttttatcggtgtaatgagtaaaatataaataattttttttttatagttgaggttatttataggaagtatttaggagaattggtttaggtagatattagtaattattagttggaattcggttattattattgtatttttttttagtattgttaggggtttgggaatttttttttttaggtatttttatattttgttaggtatttttttatttattggaatagttttgtttatatggttttaataggattaagagattggcgttaagtttaaggggtattattattatttgtattatttagattgtagatgatgatagtaagattagggaatagtgttatttttagatttatttatagtattattagtgtttatgttgttatggttaattttgttatttataatagtattatttatattttagattttatacgagagggatttgtaattgttcggttggtgttattgttaatagtattattagttttgtttttaataaaaaggattatttatatgatgattgtaattatttttataggttttaaggttttgtttatattgtgtaggtttatgttgttttttatgaagtcgttaagggggtagtagagagttttttttttgttatagttaaagttttggttttttgtgtttttttgttgattttgttgagtgttggagtttagtttttatgggatagattttggttgtcggttgatgggtatttagtatggaggtggtgggtgttgttagatgtgtgtatatacgtgtatgtttatatatatatatatatatatatatatagagagagagagagagagagagagagagagagatttggttttaattttttgggttgggtttatgaagattgtgagggagatggatatttcgggttagatagaaatgatgtgataagaggaaagttttttgaatagtttaagaaatttaaaataagggtagagatatagacgttggagttgaaatagagagggggatgttgtggcgttaagggaacggatcgtatgattgttgagtttttagggttagagttggagaggtgtggtttgttttggaggaagagaagaggaagtggagatgagtggtgtgtgggatagagaaggttatagaattttggatttttgggttttgaggttggtatcggatgtggggaagtttgagtttttttagggtttttagttgtaattgggaggttgaggttcgagattggagagatagagtttttttagaattgtacggggttatttttttggttttattggttagtgatttttatttttggggttaagggtaggttgttttttatgttttagagtagttaggtttttattggttttaggttttttttttttatttttttattgttagatagaggtgaaagggtggtttagggtgttggttttaaaatgattttaattatatgggtttttgggttgttcgttagagagatgggagttaggtttgtgagtagttttagttttagtttagagagaagggagggagggtattagaagggggttaggtttaggttttggtatagtttagatgtatggtaaatagtttttttagtattcggtgggtattggttgtttttgtttaatggtttttattttagttttagaatagtttttttaagtattttaatgtttggagttggttttcgtatttttaatttggttttattaagaattttttttttttgaaaggttagagagaggttttaagtatttggttgtgttattttttttttaattttgattttagggaatgatatttagatatagtgggggagatgggggttatgatggtagggaggtgggaggatttgggaaaagtaggaaattttttattattttaggtttaggaaagtgtagggataggtatgaaggtagatgttgagagggtagggaagatgggggataaatagtgggtttggaatgggagtgtatagttagtggaataaatttgatttttaggaagttttgtttgtagtttaatttttaatttttttgttgtattattggtttattttatggtatatttggatttttatgtttttttaagtttagtttaatttttaaagatgatgtagttttgagtttttttttttgggagtatattgttttcgtgtttgatttggttttattagggatttagggttagttttatttttttttgagttttagtttttattttttaatgttataatatattttttttatgagagtgtaagtgtttcgagatttttggaggaatattttgggttttagagtttttgatagttaggggtaggaaggggtagttttttttatttattttttttttttttagtttattttttagtttaaggtttttataaaaggtagggtgggggaagggaggggttggatagaggttagattggtagagataggggttttgttttaattgatttttgagttaagagtttcggtgggaaagttcgtgttaggtagaaaggagaggttgggaagaagaagggaggaggtagttaaggaagggggatggagaatgttggggagtaaagagaggagtttttttataaataatggtggggaggaagtagaggagagggtgggtttaggagaagttgtgtttatttgcgttcggtttaggaggtttttttggaaaaggttaggtgagtatggtgtttttggaggtagggatagtttagaaggtagaagaggttaaagagatattatgggagaaggttttgggttcgaggaaggagtttggtattgggaggagtagatggaattttgagtataagagtatttattatttatgttgtaaattgagtttttattgtttgtttggtgttatgtatttgtataggagttttaagatattaaatatttttaaagtatatggttttattttaatttatgagatagtaatgatgatgtttttttataagtaagaaaataaaggtttagagaaggtaaggagtttggtttaggttatatagtgagttgattaggattcgggttttggttttgagaagaaattatttgttgggaagatggggattagaaatagagagtttatggtgatgtgaaggtaggggatttttggagttaggatatgtgtgaatttatatgggtgtggtagggagtaggggggaagtttattttgaagtttaattttattttttttttttatgtttttttgttagtttgtagtatggtttagttgtttttgtttttgttggtagttttggttttggtttaggtttttgtagttttagtagatgttttggaaggagatagtttaggtaagtaattttatttggggttatcgtttttgttttgtttagttttaattaattgtttttaggtttaggggttttaggaagagagagaattggagggttgtggggaagcgtttaggttggaatatttggaaataagggttgagttgtgagtatttggtgggttggagaaattatggggtaggattagggaagggtattgggtgtaaggttttgggtttttaaggtggaggataggggattttgaagttaggaggtgggaaaggggtggaattgggagaaattttgaaggaggttttaaagggagtagattgggaaattgggttggaggatttagggggcgaggttggaggattttggggtgtggtttaggaattttggagaggaagtggaagattttgagagtagggtaaggaattttgggggttgaattgattgggagattttgggggatgggtttggagatcgtcggggtggttttggaggatttagaggtagggttgtaggattttggtttttggtttttggtttttggttttaggggggtcggggaattttggggtcggaaggagggattttggagcggggtttggaggttatcgggtgggattttgagggtcgatagcgttaagttttagtcggttttattcgtttatagaggatcgcgtttttcgcgtgcgtatcgcgggcgacgcgttattgtagggcgtgttcggcggcgtttttattattttttgttacgtttattatttgcggttatcgtcgagtcgtcgggttgtgttgggtttttcgcgggttaagtggatttttttgtttcggggtcgggaggtagaggtgttggtggcgcggggagtgcgcgttaaggtgaacgaggtttatcggtttcgcgtggtattgtttgcgtatttagcgtcgtttatcgacgtttttttggcgttgagcgagttgcgttttaacgatttaggtatttatcgttgtgaggtttagtacggtatcgatgatagtagcgacgttgtggaggttaaggttaaa

SEQ ID NO: 122 - DNA, 5414 bp, Artificial Sequence
  Synthetic
tatatagtgaagcgcggtgtttttcgagtttaaatgaaattttaatttaataatttttttttggtttagttataatttgtttagagatgttgttttattttttttaaagcgttattcgtattataattaaatgatatttaagtttttaattttgatttattttattttttgaagtttgagatagagttgtataatgttattttttttttgtttttttgaaaattattttggttgttgttgaggtagaaattaaatatttaagtatttatttgaatcgttcggtataagttatatttatttacgtgaatatttttttttttttattttatgtttaggtttcgttgagtttatattcggtaatattgttgttaggagtttttttcggttattatttattatttttttttatataggggaagagaaagggaagttcgagaggatttagggaaagtagaagggggttaaggattatggatagagttcgtcgcgcgttcgttgttgtcgttttttttagtattttggcggtttttgaggatagcggttttattttgaaatcgttatttcgttcggttgaggttaggggtggataggcggttttttattttttatcgtcgttttcgggagttgattattcgagggttttttttttttatttttttttttattttgtttttgttttagcgcgcgttagcgttttttaggtttgtcgtttgttttcgtatttgttcgttttttttaggcgtttagtgtttgtatttgttttcggttaattttcgttcggattgggttattcgcgggtttttgcgtcggggtttcggggtttttttattttcgtttgtattttgtttttttcgttttttagggaggtgatagtagtttttaatatcgcgggaagtatagagaaaatgggatttagaaggagaggaagtagtgtgtgtgtgtgtgtgtgtgtgtgtgtgtgtgtgtgatagagagagagagatagatagaaagagattatttttttttgtaattggaattaagagtgtgtgtttatttttaggaaaagtggtttgtattgggattgggatagaagtgggagtgaagtgttagttaaaaataggtttcgtatcgagaggttgtggaaatgaagataagtgaggtttgtgttagttttcgagggtgtgtgtgtgtgtgtttgtgttgtggggtgtatttagtagtatatgcgttgtgtaatttttgattttttttttttttgttagttgttttttttttttttgattgtggttaatgaagaataataaatttaggggtagggtttgttagtggatttttttaagatttaattcgaattgtattggatatagggaggaggaggaagagaaaaggggggtaagaggagcgtgtgtgtgtgtttgtgtgtatgtgtgtgtgtgttgtgggaggggtggggatagcggggagggggaggagtcgtatgcgtatagacgattcgagtttgtttcgcggttgtttaattcgttgagagttgcgagaaatcgagtgagagaaagttttgtagttttttcgattttatgtttttttggtattaggtattcgtcgggtcgtggggggttcgtagtcgaacgtcgattttcgttcgtattgggttgggagtttagagtcgcgcgtagaattcgggttggtcgtaacgtttgtgtttttagcggtggtcgggaatttgggattagggttatttgagttgacggggtgggggcgggtcgagtggggttggaagtttggaatttagtggtaagtaggaggcgtaggaggtggtagttaggtaagaggtatttttatttatttaacgttggtttgggtcgtaattttatttgggagttttttttttcggtgagatagagattcggtagaagaagcgggaggggttggaggttggtttttaggtaggtattgttcggcgattggagcgcggatttggttatttgggtggggttgagtgggggcgcgattgtgagtagtagtcgcgggacgttgcgaaggggcggcggtaatagagtacgggcgggggtagaaaagaggcggcggagggcgcggtgggggagcgcgaggcgagtgttgagagagtagaaaggatttaagtttgaggggagtagagaggaagaaggggtaacgcgagaaatcgaataggagtcggcgttttttggtaagggagggcggaggcgcgcgggagagagggagagagggagggcggggggcgcgggggtaggcgcggggagaggggagtataattcgtcggtcgcgaggagcgggggtagtttcgggtgtcgaggtttgtagttagcggtaagcggagttaggtattcgtttagattgatagtagaggcggcgaaggagcgcgtagtcgagattaggcgtatagagttcggaggcggcggcgggtgagtttaatttcgtatagttttttttagttttagtttcggttggttcggtatttttcggagggtttcggtagtcgggattagtgagtgtttttacggattagcgtttcggcgggcgggaagatgatgatgatgtttttgaatagtaagtaggcgtttagtatgtcgtacggcggtagtttgtacgtggagtttaagtattcggtattgtatagtatttcgtcgggtttttcggtttttatcgcgttttcggttagtttttttagtagttcgagtaacgttggtggtggcggcggcggcggcggcggcggcggcggcggcggaggtcgaagtagtagttttagtagtagtggtagtagcggcggcgggggttcggaggttatgcggagagtttgttttttaattttatcggtgcgtatttttgtataattatcgtttaaaggtatattttgatagttttttttatttgtttgatgttttttttatgtttgtatagtaaattattttatatttttaattaattttttttttttttttttttaagtatttagtaggttttgttttttatattaatttttatgatttgggatgttgtttgtgcgcgtgttgtgttgtgtttcgttgtgtttataggtttattttttttttttttttgtattttcggtttttttttgtggtttttttttttttttttttatttttgtttttaggattattattattattattttaacgatttgggaatgttgtaggcgcggcgacggtgtcgagttttgggtcggggttttcggagagagggcgtataattttttgttgagcgtaatgtgtgttttttatttataattgtagagtaatatattcggcgggttggatgagagtttgttggttcgcgtcgaggttttggtagtcgtggatatcgttttttagagtaagagttattattattatttattttattatagtttttttaaatcggacgttatttattatattatgaatattatttcgtgtacgtcggtcgttttttttttatcggtgtttatttcgtattttttcgcgttggcgggtacgtattattattattattattattattattattattattaatcgtattaggcgttggagggcgagttgttggagtatttgagtttcgggttggttttgggcgttatggcgggtttcgacggcgttgtggtgtttacgtcggtttacgcgtcgtatatggttattatgaattttatgtattaagtagcgtttagtatggtttacgcgtacgggttgtcgtcgtatatgggttgtatgagcgacgtggacgtcgattcgcgggatttggaggtattcgtcgagcgttttaagtagcgacgtattaagttgggggtgatttaggtagatgtgggtttcgcgttggttaattttaagattttcggcgtgggttcgtttagttagagtattatttgtaggttcgagttttttatattgttttataataatatgatcgcgtttaaatttattttgtaggtatggttcgaggaggtcgagaagttttatcgcgagaagtttattaagtttgaattttttaatggcgcggagaagaagcgtaagcgtacgtttatcgttgcgttagagaagcgttcgttcgaagtttattttgttatttagtttcggtttttttttgaaaagatcgtcgttatcgcggagaagttggatttgaagaaaaacgtggtgcgcgtttggttttgtaattagaggtagaaatagaaaagaatgaaatatttcgtcggtatttagaagatttttggtttttttagagacgttttttttttcgttcgtttttttttttttttttttttgtttttttttatttttggcgattagaaataattttagtaaatgtgaatttcgataaatcgaggattgaagagggagcgaacgagcgaataattgagtttaagtcggtgagaatgtgaaatagttttttaaaggaaagaataataaaagatggtatttgtttgttgtagtaaagttgtttttttgaattttatttcggtttttttagaggaagtgtggagatggttgtttgtaggaaggtagacgagatagtgtttaaaaagtttataagaatgattaagtaagatttgtttttatttttatagatattattcgtgtttaagtttaaaagtatattttgtaattattttttagaaatagaaattgatttaggattaaaattttaaattagagttgatgtttaatgtgatagagatatttttaaagtattttgaattttaaaaaaagatggtagattttttgtatttatattgtatattatatatatatttttattgtggtttttatttttttttttttttttgaagtgttaatgtttaagaaaagagttgcgtttgttgtgtttattgattttgaaagttattattagattattgtagaataattttttgtaaattattaatttatttttttagtaatttaattttgtgtatattttaattaattaaatttttttcgtttaaaaaaagtgggggaaatgtatagttagtaacgtttaaaaaattttgtttgatgagtttatcgaatttttatagttttttttttatattgtgttttttttgatttatttgtatatttttatttgaatgaagattgtttttttttttgtttttattggtagtgttttgatttgtgagtcgatatttagtaatggatgttttaatcgtgtagatttgatttattgtttgaagtattgtttatttcgttatatatttaatggggatttttatattgtttttatgatatatgagcgtttttatttatttttatatatatatatatatatatatatatttttaatagaagggaagaagtagttggaagtatgatcgatgtattattttttagttttaggtgtatttgttatttggtgtttgttttttagattttagattttattaaggtattttagttttttagttt

SEQ ID NO: 123 - DNA, 3381 bp, Artificial Sequence
  Synthetic
tttattatttttggggagtttttttggggtttatagaaaattttattttatttcggtttttttagtatcggtgatattttggaattcggtttcggttgttcgttaaggaggatttggtttgggggcgggagggtgttttagagtttttgggggtttatcgtattttttattcgaggtttttcgcgagcggagtcgtttttttaggtcgttcgtttttttgtcgtaggagtaggttcgcgttcgtcgttcgagagttttcggtagagggagatattttagcggttttttcgatttggtagcggtcgggtcgggagtgggtgggggtgatggggggtggggagaaaagggggagtggggagaggggaggtgtgtgaagggggggttcggggggcgggtttttgtgtcgttgacgtttcgagtagtgttgggaagtataggttgtgttgttacgtcggtgttagtttgatgaagattggtattaggtaagttgttatttatttttatgttagagacgtttttgtaggcggcggcggcggcgcggcggcggttgttgttgcgggcggttgttttagagcgcgtgtgttttattttagtttttaagttagagtattatttattgcgatagggtaaggaggagagagggagagagggaggtagtagggaggagagagagggaggtagtagggaggagggaggtagggagtagcgagggacggcgggagcgtgtagagagaagttggggaagcgtcgggagagcgcggagcggagtagcgcgaggggcggcgaggtcgggtacggaggttgcgagagtttcgcgggtcgttcgtttttttgttcggttagcgtttagtttcgtcgtcgtcgtcgtcgtcgtttcgtcgcgtttgggttcgtggtcggtcgcgtattgttttcgggtgtaaggagtcgggttgcggattcgaatcgtcgcgggtttaatttcgtagcgggcgggcggggagttgtgcgttaggagcgttaggggattcgagaataggaataggtacgtcggttcgagttcgggtgtagaaggttttcggtcgggcgtttcgcggggagaggttgggaatcgcgggtaggttttaggttttttttttttcggtttcgggcgtttttggggtcggcgtttatttcgcgttattagtttggagatgttttttagcggttggaggcgggagcggttttaggttggggttaggtgatcggaggagtcgaggagtcgcggtcgcggtttggggaggtagggcgaatgagggtttgtagacggattagtggagataggggaatatcggcggggttcgggaagttaggattcgaggggttatacgtacggatttttatttagggaggagcgagaatgtgtagggttatcggttttttatgattgttggggatttttggtgaggcgggcgtaggggttcgcgcgaggtttgggaatcgggagtttttttggttcgagaattaggggatgagttcgtaaaagagggaattgaaagcgatcgagagcggagagttgagggggatggcgtaggatttcgaatttgtcgtttaaagtttgtatttttttcgggttaggagacgggtttttttggttttcgatttttaggaagaaacgagcgaaatgggtcgttttttttcggggttttttacgggggagtcgggggttttcgcgcggtggggagattcgggttgggaattgaggggtaggggtttgtggatcggtttgaattagggttgggttttaggattagtttagagttcggtttataggatttagattgtttacggaatcgggatcgaggggtcgataagtagtttatacgtcggttagagtagagggttggaggtcggagttgggggttggaggaacgggtggcgtttttaggatttagtaataggattatagtttttttttgtggtggaagttattggaatttggggagggtagtacgaggggttttgtagtttcgcgtgtgaaaaagcgtttaggtaggcgatgaaagtagttgatttgagttatggtaggcgagtttcgaatttttgttgtttttttttgaaagtgtttttttaggaggagaggatttgggttatataggattcggttttaagagagcgatttcgggaagcggatagatcgaagagattttttgggcgaagcggtagggtagtttcgcggggttgggagtggatttgaggtttcgatttaggcggttcggagtgttttaggagttatttgggtttgcgggcgtagcgcggcggggcgggagcggtggttcgtaggggtcgcggtttgcgatgaaggtcggggggtagcgttagtagcgaggtgttatagtgggtcgaggagtttgggttgtggtttagggtaggatcggtttaaattttagtgttttgattggagtcgttttttgtgtttattcgcgtcggattgagaagtttataaattcggtttttggtgcgttcgggggaggaggaagtttggagtgttttattgttattttttttgttcggagattttaggatattagtttttcgggttttaaatgttttataatcggtggtagttaaaagtttcgtattttaggtagtttagacgatttttttcgttttaaatttgagaatgatggtgtttaaatataatacggtgtattatttaaagtttttgtttttgtttttttgttttttttgtcgttttggttttcggaggttttaggattttggtggaagcggggagggagggtttcgtttttggttttataattttttattcgggttgtatttttatagtttgggtttagttgtggggtgtggggtgggaaggggtttcgattagggaggtaggttattttttgggtatttagagaaagagtgtgtgtggggggtgggagcgggggggaggagggtattgaggggaggaagatttgagattttttttttttaatattaaatttattattttagttttttgtatttttggtagggagagttagatagtttttataggatacggaagacgtacgatttacgatttacgagtggtttcggcggttcggggcgcggcgattgtcgtcgcggagttttaaggttgttgttttgtaggtttggaattcgtgagagttttttttcgttgagagttcgaggggggagggagtcgtgattttacggagattttttggttatcgttttttttttgtgagcgggaaaggtttgaaagttaagagaaagagaggttgtttagaataaggggtagggggttatcggagggagatgggttcgattatcgtttatagtaaaatgtttcgtgcgaattgtattgaagtatatttaatttgagattggttgttttatgattttttttgg

SEQ ID NO: 124 - DNA, 1266 bp, Artificial Sequence
  Synthetic
ttttttattagatttggtcgcgggaaattataaaggatagttaagtgatttttaaggagaaagtttttggtttatttttttttcgtcgttagatttgtaggtttttgggaggagttttttttaaattttgaagattgttaggtttttggcgaacgatttattttttgaagatttagagtggtggattataggtattgaaaagtttttggtggttttggaagatgattttggtgtgagtttatttttttaggttgggggattaggtagaggaattttttttgttattttttgaaagaagattagtggggaagacggggtttgaagtgtggattaggagattttacgtttttgtttattatttttgtttaaattataaaaagatcgaggagtgtaatgaattttaggaattatgtatcgtttttttgaagtttgtttaggaatttaatttttggatttagaaatttttgtaaagatagatttattgagttaggtagtttgtattagtatttttgtttttaagattttgtttcgtttttttttattgagagattgattttttgagtgatttgtgtgtttttcggtaaatgatggagacgcgtaaatcggcggaacggttggttttgttatattcgttgcgtatcgcgtttttgggcgtttcggggattttgttcggattttcgcggagggatttttttagggtcgttttgcgtttggacgtcgcgtgttgggagtgggcgcgtagcggttgcgtacggggatggtagtatttgttcgtgtcgttggatagcgttttcgagttcgttttttttcgtaagcgtaacgagcgcgagcggtagcgggtgcgttgcgtgaacgagggttatgcgcgttttcgagattatttgtttcgggagttggtagataagcgttttagtaaagtggagacgtttcgcgttgttatcgattatattaagtatttgtaggagttgttggagcgttaggtttgggggttcgagggcgcggtcggcgtcgttttttagcgtagggcggaatgtaatagcgacggggagtttaaggttttttcggcgttttcgtttagtagcgagttcgaggaggggggtagttagcgagcgttcgaattggttaggattttcgcgttcgtcgtatagcgcgtagtcgggcgtttaatttaaggtttttttcgaaggtggtttgtatttttaatttggtattttttttaggtttaaattttaagaaaaagaaatgggtgttggggtttgggggatg

SEQ ID NO: 125 - DNA, 2968 bp, Artificial Sequence
  Synthetic
ttgtagtttcgtttttttaggtttgtaaaggtagacggagaattatattggaatttaaatcggaagtttttaaggtattttaaaaatattttttttatttttttttttttgtagatattggagaggttggtaaacgggtttttttgaagatagaagaatgtatgatttaatgttttttttagatttttgtatgagtggatgtatagtgtttcgtattgtgtggtggggcggggtgtgtttttttattgatgaaatatattgcgtaggttaattcggtaaattgaaatgagaagagtcgattgcgggggtggagggggtgtggtattagggtgtcggcgtttgtggaggggggcgcgaatgtgaacgtgtgaaagcgagaggcgtgttaggagagcgcgggaaagtttattggtgaggtaagtgtgcgtttatttttatggcgttttggttcgcggtagtttttggttgggcgaggggtgtgatgtgggagtggggtgggagggggtagtaggcggggtttgttacgttatttggagagtgtgtgttgggaaggaagggtagagcggagagtcgagtcgttgtagttgcggcggcggtagcgaagttttgagtcgtggggaggtgggttttcgcgttcgggcgtcggggtagtttcgggttttttgcgaggtttgcggcgcggtttttagggaggaggtggcggttgtggcggtcggaatcgcgattttggtcggatttagtttcgcggtggacgtagggcggaggtcgagtttcgttaggagtttttgtcgagtcggagggaggcgtatttggcgtttcggtattagcggtagtcgggggttcggagcggttggaggagcgtagtgggaattgggaagagttagttcggttggagggcggatttttgcgttcgggagtcgggttttaggtatcgttgggggcgaagttacgcgttttttcgggtagttaattttatacgcgtttgtgtgcggtttcgggtattttagtaagttttagtattcgggcgcgggtaacgggaagcgtagaattaaatttttagcgtttaggttatttttttagatttagttttgtagggattagggttttagggtttacggatttaacggttaggttagatcgcgaatcgggaggagcgcgggttttattttaaagagggcgtagtcgggagttggggagcgggtgtcgcgttttagagattgtgtcgtgggcgtcgttttagtggcggggagcgtattttcgagggggtatgagatcggagaaattttttacgttggcggcgtcgggggaggttcgtgggtcggagggagagtaataggatgcgggagatttttcggaggtcggcgggggcgggggttgttgtagtagcgagcggttggtgattaatattttcgggttgcgttttgagatataattgcgtattttgtcgttgttttcggatacgttgttcggagattttggtcggcgagttcgttttttcgattttttgaggaacgagtattttttcgatcgtaatcggtttagtttcgacgttattttttattattattagtttgggggtcgtttgcggaggtcggttaacgtgtttttggatatttttttggaggagattcgtttttattagttgggggacgaggttttggcggtttttcgggaggacgagggttgtttgttcgaaggtggcgaggacgagaagtcgttgtttttttagtttttttagcgttaggtgtggttgttttttgagtatttagagagttttgggtcggttaggggtatcgttatcgttttcgtgttggttatttttatttttatagttattttttgtttggagattttattttagtttcgtgtagatggtcgaggtggaaataatggtggtgtgagtcgagtttttttagtttttagggggagttaggaggaagaggaggatgaagacgatttttatatattttattatggtattatttttggggaaatggggatcgggggtttttttttatttagtatttttgggggttttttttttatagattttttttttttggtggagacgttgtgtattgtttggtttatttttgagtttttggtgcgttttttcgtttgttttagtaagtcggttttttttcggaatattatgaatattattgatttggtggttatttttttttattttattattttgggtattgagttggtgtagtagtaggagtagtaattagttagtggaggaggcggttagaatgggtagtaggttatgtttttggttatttttcgagttattcgtttggttcgggtgtttcgtatttttaagttttttcgttattttaaggggttgtagattttgggtaagattttgtaggtttttatgagggagttggggttgtttattttttttttttttatcggggttatttttttttttagtgtcgtttatttcgtagaggttgacgatgacgattcgtttttttttagtatttcggatgttttttggtgggtagtggttataatgattacggtaggttacggggatatgtattttatgattgtggggggaaagatcgtgggttcgttgtgtgttatcgttggggtttttattattgttttgtttgtgttcgttatcgtttttaattttaattatttttattatcgggagacggagtaggaggagtaaggttagtatatttacgttatttgtgggtagtttgcgtcggatttgagggtaattgataacggatttggtaagtttgattttttcgaggttaatcgggaacggagatttagttatttttttatattatatcgggtttatgtagagaaaagaatgtttacggaggtttgatttatgtaggtagggtttgtaggaggggagtattgagttaatagttttttaggttttttttttatttttattatttattttagttttagttgattttttgatttttttttt

SEQ ID NO: 126 - DNA, 4899 bp, Artificial Sequence
  Synthetic
gaggtagttttgggaggtattggtttgaagggttagatttttggtaagaatttttggaagaacgtttgtttttcggaattagatagggaggcgtgtaacggggtggggtggggggtatttttagttgtaggtatttttatttttaattatttgtaagaggatatatatatatatatattttgggtgttagagaagtttttttttttttgtatttttttttttttaagtttttgaaatttagaagtgtttgtttttaggcgttagttttgttgagtggatgtggggatggaggttattggaggtttagggttggtttgttgtttttgttttttgttaggagtattgtgaaggttttgtagggttagttttgggaaattttaggtttggttttatttgatttttttattggagcgaggcgtgtagttaagtttgtttagatttttttaggttacgggttcgcggtagtttagtttagtatttgtaggcggggggatcgcgcgtttggttcgaagatttcgttttgggtaggggatagaaaatatgaaattagatggggagttatgaataattgttttttttttttttttgtcgtgttttttgaattcgtacgttatgtaggaggggggcgggggcgggggcgcggggaggaggagggggtcgtttgttattaatttgggaataagcggttaatttcggtgattggtattttcgtttttttttttttattttttttagtgtggggaaagtagttaagttcgcgcggagcgattgtgaggggttttgttggaatttggtagcgcggaggtttggagagaagttttatgttggtttttatttagtcggttcgtttttttcgagttttggaagttttatttagtcgtgtatttaatggttttataaagttgattataagttttagcgtatttttgaaggagttaaaagcgacgtaggtgtaaacgagtcgagggagttttttatttcggtgatagaatgggataagttgggaaaggtttagattatataagtttaaggtttattaggtcgtagaaagtttgttttgggaatcgggggttattattcgttttatttagcggggttcggggattttgggggtcgagcgaggttagttcgggcgggagtatcgtcgttagcgcgcgtcggcgataggggggaatttcgttgggcgttgcggttaggtcggggtttagggcgttgggttgtgcgtttgtataaatttgtgtttcggcgggtcggtgttaattttagtggggatacgggagaaagagtacgttagtaggttacgcggcgcgtgggtagtgcgtaatttttgtcggcgtttaggttgtacggttacgttttagtcggtgtttttaggttatggatatttagttttagggtcgttttgcgttttttttttttagtttattttttttattggtttttttttttgttcgagtggtagcgcggtttcgtagggagcgatttcgggagggatagtgatcgtacgcgtggagtggggacggcggggtataggacggtagtttaagagtgttcgtatggtcgaggacgttttcgttgcgagtttggggtcgaggggtagttttttttcggagttattaatttcgttttttcgtgtttagggattagtaatttggtgttgcgggcgttagaaagaaaagggtagcgtttcgcggagcgcgcgggggaagagggattgcggtagcgggacgcgagggcgggaggggcgtagtagcggggagtcggagcgtagcggaggggagcgttggggggcggggagcgttcgggtagttattttgttttcgtttggagtttcgcgttgcgtttaggagggcgtaatacgtagttttcgcgggggttagagttcggagttttttagtgtatgtttttttttttcgcgttttgtcgaggtttcggttttgtttgtcgtgttttgggtttggttgtgttcgggggttttcgcgggtagggcgcggggtaggtagggcgcgcgcgtcgacgtttttttgtttgataattaatttgagttaatgcgatttttatgtaaagttaatagcggataattgtttattttttcgttaatagtttttattataattatttatttggaaatttgcggttggattaatcgttatattttcgagatgagcgtcgttgtaattcgtagcgaacgcgggtagggatttggagacgtttttcgggcgttggcggggtcggtgtgggttagtttcgttatcgtttgagacgcggtgtgtttaggtgttggtgttcgcgggagagggttgtggggcgttttttttaggatttgtttgatttgtttttcgttcgagtgggtggtgcgcgggtgcgggaggcggagggaggaggaggggtgaggggaggagggtttgtggggttgcggattcggagtagtttgggtggagcgcggtttcgggaggttttgggtgtatcgcggcggggtttgggggggtttaggcgtcggaggagtcgtcggtgtcggaatgtagcgtgttttatttgggagaaacgttgttttcggttttttgttttttttttgttgtttcggtttttttggtttcgtttttcgttttatttattcgtttttattttaggttttttttttaggattttttttttaaggatttaggtttttgaaaattattggtaatagttatacgttttacgattgaaatttataagagaagagttttagttttgtaggtcgttttagggttaggggtagagatggtggtaggtggtgcgttaattttttagggaagaggaatttgtattataaagatttgtttttttgagttgaagttaaaacgggggcgttaagcgcgtttcgtttggcggcggtggaggggtcgcgcgttcgcgttgttttagtcggagttgttttggttggtgattggaggtttaacgttcggaatttaggcgtttttgtagtttagatttgtcggttaaggggttttagttgtaattttttaaaatggtgtttttggaaaataataaatttagatttaattggtgatagtttttggttatagagaatgaaattgtttttttttggcggtggaatttttaaatttcgaagagtgaaagaatataatgaaataaaatgttataagattattggattttttagaaaaaggaagattttaaattatttttaaaatgaggttttgtaaatttttgttaaaaatttttaaatttcgaatttttttttataatatttgatgagtgttttaagagtaaacgagtaaattttatttcgagaattaataaatttaagttttggttaaggtttttttcgcgtttttttttcgtgatttggggaatgtttcgttttatcgtttatttggtttttgttatttcgtttattttgaagtgattcgtggataatgttgttttttgtcgtagtttcgtcggttatttaggtagaacggggtatgtaaatggtttggagaggtcgaggtcgaatattgcgtttattgtttcgggcgtttaaagcgtcgagtagttgcgtagattttttgggttcggattttcggtcgttacgttaatgttattattgtaaaatattagattttgagaattatgtagtttgaatttattagtagttttttcggcgaaggttttttttttcgtgataagttttaaagtttagattttttttaacgttggtaggggtaggaagaggaggaggggtttagggaggggggtttttcgtaagtggggtttcgggggatttttgttttttttttggtgtatttcgtaattttaaaagttttttttggggttttgagcgcggatgtttgatttgaatgatttgagatttggatatattttcgtttttttttaagagttgttttaatgttgcgagtttaaatttattgaaagaggtttagatattaagtcgagtgaattttatagatattattttttgttttaatgatcgcggtagtgatcgtttttgaagttatattttttagggtgaatttataataatgtatgttagagaggttgcgcgttttaaagtaaaatagttaaaatattaggttacgttttttaaattgtaggattgtaagtagtttataaatttttagtttttattttaaggtgaggagagttttagtgatttgaggatttgtttgtattttcgttttttacgttcggttttttattcgtatttgtttaagtggtttcgtttttttagaatttgaatggtggggaggggtgtttttgatgtttcgggtgtagggcgggtattagcgagcgagatttaagggcgttagaatttaggagcgtaaattatcgttagggtttgagggacgtagaggcgggtggagtagtgttttttagattcgtgggaggtcgtaggcgttagtagaggtaattttttttttttgagtaatgttagttttttttaggtttaggatttttattaacgtgtttttttatttttttataggattttaagttgttgttttttttttgtcgtatttgaggttgggaaaattttttaggagaaggtaagagaaagttattagattagagtcgaggattaaattttaaggtcgaagacggtagaggggtaggtttttttttgtatattttaaggttttttttgtattcgcgaggttttttttgagcgtttaggttttcgaaatgtttgttttttttttgataaaaggagggggtaggatgtgaaggggtagtgtaattaataatgtttttgtaaatataataatagggaaatatatggaggaaatttatttattgggtgtgttatttttggtggggggcgggattgaaagtggcgttatttagatttaggaaaaagttacggggtgggcggggtaagcgggggatttttgg

SEQ ID NO: 127 - DNA, 2346 bp, Artificial Sequence
  Synthetic
atttattttgtgaatattcggatatggtgaattggtattggtattgttgttgaaggataatttagtcgtgtttttagttatggttatttaggtttgttttgatgtagggttttgatttaaggtattagtgtggttttttagggaagtattggggatcgttatttatgtttgttttggatatggttatcgagaattgttttgtaggtatttatttaggaattattcgaagtggaattgtttttggatacgttttttttgtattttgttttttttttttagtgtttttgtgtgaagaagttttttttatttagttttcggcgattttttggtattttggatagtttttcggggagtagtttatcgttaggcggcggttgttaagagaggaatttttttgacgcggagtttgtcgtttcggggttcgtttttcggtaggttcggggagaggtggggtgataatgggttggggtgcgcgcgtgttttataggtgcgagatagagcgagtcgtcggggtgtgagttagcgcgttgggggttaagaagttgggtgaatagttacggaattttatttacgttcggtttttttatttatttcgtttatagcgcgtgttttagtttagggcgtgcgtgcgttcggtgttcgatttcgggttgtgtgtgtttatttggcgagatgtcgagagcggggggagtgtttttgtcggtgtatttgggtttaggttaggggattttttttttttattttcgcgtgggtgtgggggtgtgttcgggttagggcgcgtgtgtttttgtgtttgtgcgtgcgtgtgcgggttagggtggtgggatcgcgtattagggtagggtgtttgcgtttgcgtttgggtttgtttggtttgtatgtcggcgcgatttcgatttggattcgtgtttttggatgtcgagaggttagcgtggtgggggtgtttagtttttcggaggagtattatgttttgatattttcgttttatcgttttaaagttggtttggggtttcgtagggagtggtttgtatggggagggttcgcgtgttgtgtttttgggaggggtaagagagtgggggcgtagggggcgggttaggtttttgggcgcggcgcgggttcgggggattcgcgcggttgacgttaggttattttttaaatagagtcggtagcgcgtttcgttcggtatttttcgaagagttagatcgcggtcggcgttagcgttatcgttcggtttattcgttagttcgtatagtcgcgtcgtcgtcgagcgtttcgtgagcggcgtttcgaggattaggaatggggtttcgggcgttgggcgcgtttcgaattcggcgtacgtaagagtttgggagcgttcgagtcgttcggttgttcggagttttatcgtttaggatcgggagatgttggaaatgtaatcgtttgtttttcgaggagtcgttgttttcgggattttttggtattgtgcgtattttggttagtagttttcggagaagacggcgtttttaacgttcgattcgcgtggtcgtggtagcgttacgcgagttttttaggcgatcgtagggttatagtagtttagtcgtcggtgttttttcggaaattatgattttcggcgcgggtttatggagttatggtttatagggttttgggtcgcgcggggttattttagtcgcggagggcgcgtaggttgtttttcgtttttacgttttcgtttttttgtatttatttgtgttatagttttttgtgttgttgcgacgatttgggtcggagtcgttttttcggcgcgtttcgttgttttcgcggttttagcgcgggcggttagaaatttttttagaagtttcgtttttgtgattttttcgggtcgacgtttagcgagtttagcgtttttagcgcgttcgtcgtcgtcgtgttcgtttttcgttttttcggttttaattatttcggtttatttaagttgggtattaagcggttgttttaagtttttattgtgggcgtgaagaaggggggtattcgggtcgtgttggagtttattcgagtatattcggacgtgcgggttttgggtacggaattttatttttttgataggaattacggtcgcgggttggattggtataggtaaggattaggagtttcgtttcgtgcgtcgggtttttgatcgtttttattgggagagttattcgttttttgtgtttttttttttttttaatttaatttattgtatgggtttaggttgatatatagggttatggggggttatagtagaatttatttagaattttttagtgataatttagacgggtagtttttggaattgtaaagggcgttttttcgttattggagtcgttg

SEQ ID NO: 128 - DNA, 2695 bp, Artificial Sequence
  Synthetic
gtttttttttacgggggtttcgaggtgttttgaggagggagaaagagggcgttttttttggggagtttatttttcgggtttttattgatttggttttcgttttatcggttttttgcggtcgttgtagaagcgtattttgttgaatatttcgaggacgtgtttttcgtatagggagcgttcgtttttgttggggttggagcggcgtttggaggtcgatattcggtcgttgttggattttttcgtttgtcgtttttgtcggattaaggagttggttatcgtcgtagttatagttgtttagcgagggttttaggttttagtttttattgcgtttttcggtttgcgtttcgtcggggcgagggtaggatttgggcggttagggaaagggtagtcgcggggaggtagtgttaaaatttgaggaggttgtagtatcgaaaattcggcgtttataaggttagttaaagtttgggtagtggcgataaaatgtgtgaaaatttagatgtaaatttttttaatttttggcggtcggggggcggggcggggcggttttaggtttttttgcgaagtagacgtttgtattttaaatttgtattttaaggcgatcggcgtttaaggggtagtggggagtttagttatattgcgttcggggtattaagtggaaggggaagaacgatgtttaaaataataagacgtgtttttgttggagaggcgtaagcgttgtaaggtgtttaaagtatatttatatatatatatatagaaaattcgtttataaagtagagtttggatttaggcgggtagcgcgttttcggtagaaaatattaaaaagtgaataaaacgtttttttagaaaataagttattaatcgtacgagagaaggagaggaaggtagtaatttaattttttgcggttcgcggttttgaagattaggaggttcgttttagtagggtgaggtttatagaatgtatcgcgtcggttgcggttttttaggggtcggttattcgagttttggaatttcgagaggcgcgaagtgggagcggttattcggagtttgggtaggggcgcggggcgggggtagttgtttttagttgcggtgagagtaattttcggttagtagtattgtaaagagagcgggaggcgagggaggggggagggcgcgagggagggagggagattttcgagggttaagtatttttcggggagaaattagcgagaggcgatttgcggggttttaagagtgggcgtttttttttttttcgtttgtttttcggtacgagacgggtatagttggtgattatttagggaattttaaatttggaatgatttagtagtttaaataagtttttttaaaaggtagcgatgtcgaaggtgttttttttagttcggcgtttatacgtttttaattggagtttttcgttatggtttattcggggtcgtcgtatcgagttggttttcgtataggtttagagggagcgagggaagggagggaaggaaggggcgttttggcgggttcgggattaggttatcgtcgcgttgttgttcgtgttttttaggttcgcgcgtttcggtagttagtagtttataggtagtagattagatggggattattcgtcggacgtaaggtcgattatttagtttcgcgtcgtttatttcggtcgaggaaggaagtgattcgcgcgttgcgaatattcgcgcgttcgttcgggtggggcgggggttggttgtaggcgatgttggttcgcggcggttgaggtttttggtcggagttgtttattatggtttggcgttaggggcgtaggcggggtttttaggttttttggggttatttcgcgaggtagtcgagggcgtaattcgggcgtttggggtcggaggcggaattaggggtcggggttaggaggtaggtgtaggcggttgttaattcgtttaatttgttgcgcgggtggtcgtttagagtcgcgggtttgcggggcgtttttcgtcgtcgcgtcgtcgtttttttaggttcgggagggggcgtttagggtggagttttatttatgggttgaggttttgggcgcgcggagtcgtcgtcgttttttcggttggtttagttggagtgttagtttcgtaggaaattcggggttcgggcgagagttatcgagatggtaggtgggacgtagagttcgcggtagttagagtttttttcgtacggttcgtcgatttacggaagagcgaaagagcgtttaggtggggtcgagttgggggtcgggtttttggagcgttgggaagtatagcgcgttttagttaggtttttttttttggagtttttcgtttttagattttttttttttttttttttttttcgttattttttttttttttttttgtgttttttgttttttttttttttttttttttacgtaattttacgtgattgaggtttggatgagaaatttttagaggtagagcgagggaattgtagtttgggtttgtttcgttcggttttttttataagagaaatataattatagtgggagttaaaggattttaggtgcgtaaagaagaggtgggatgggggagttgagaaaatgtagtttatattttttttaataagtttgagtacgtagaattttttgtttagttaggaagaaagtgaatattggagaaagtaaaaatgattttttggattttatcgtgggttttatttatggtttattttggaa

SEQ ID NO: 129 - DNA, 2837 bp, Artificial Sequence
  Synthetic
aataatgtttttgttgttattttagttggtttttcgtttgtttaaatcgttttttagtcgttgtttttttattttataaacgttattgagaattgtgtttattatagttttattttatttttaattttatttttaatttttaggtttagattatggtatttggtatagtttttgaattttatatagttcgtttcggaagcgtattgtaaaacgtattttagatgtttttttaaatttatttttttatttttttattttagaaaatcgttttatttatttattttgagaaagtggatttaattttcgttttcgcgttggagaaggaaatataattattttttttttttttttttttttttttttaatattttttttttttttttttttgttttttttaatttttaattttaagagtagtatttagatatgtaaattttttaattttttttatttttttttgttttaaatattgggtaattagattggagataatagacgtagggaaatttgggtttttatttgttaaggagttttttttttttatattcgtttttatgttttcgttttttcgaggggttttgggggtatttttatcgttttttttttgttttttagaaaattattcgtaattggaggttttcgcgcggtagttgtattaatagtaggtgatttaggtggtgcgaggcgatgttttgttatggtattttgttttttgtagggatattattgagggttatatgaaatgatttttagtagttttgtaaaagtaaaggggtggaggagggtttatgtattttgttttgtgttttagttttttttattttgtttttcgtaatcgaggagattttggtatttgggagcgggttgggtcgagaaggtaagattaataaagtttagtaagtagatagttgggagttgggtgttattgagttttagaaagggggagtagagacggaaatattcgaatcggtgaaatgtaaatcgtgtttattgtataagggaatcgtgtagggagagtcgttttttataaggagttaattagcgtttgtttcggatattttcgcggggtttttagcggggtttaaattggcggcggtttagtagggggcgaggggtgttgggaaacgtcggggttgcgaatttacggaagaaaatgtattcggtgtagttgttttagattttgtcgtcgcggtattggttgacgaaggcggcggtgttcgaggagttatacgttattatgtggaagtcggtttcggataggcgattaaaggtttgttttaagtaggtgaatttgaggtagaagcgggacgtgtattttttcggttgtcggtcggggtcgcggttttcgttgagcgtgttttcgaagatttttttggttagcgcgatgcgttcgtatattatgatgcgcgttatacgtcggaatttggcgtcggtttggttgtcgcgtacggtggtgtaggagtttcggtagtttagcgtgaggaagttcgagcgtttgttttgcgcgtcgtcgtcgtcgttattatcgtgcgttttcgggttcgagggtacggcggtcgtcgtttcgcgtagtagtagcgcgtcgttgttattttgcgagacgttgttttttaggtcgttttggtagttttcgtcgttgagagagttttgtttggtgattttgggcgatagtagtttgattaagtcggtgagttggaaatattcggtttcgcgtagtagtcgttttttttcggggaagtgtttcggtagcgcgagttgtttgtttcgtagataatttagtacgtatttgaaaaggaagtcgtttcggtcgatgaagaagcgcgttcggttgtttttgggtagttcgtttcggcgtcgggcgtcgttacggggattagagggcgagaatatgttggttaaagtattgttcgggacgttgagtagcgtcgagtgtttggttatataaatttggtcgtttacgtttagttttattattttagggaagggcgagggtgcgtagggtttcggggcggcggcggtcgacgcgtcgggcgagttggacgaggaaattatttcgttaatgggtaggatggtgttgtcgtcgttgttcgtgttttttagagttatagttttttcgtcgtcggtttagtgattcgagagagttgtattttttcgttttcggagttcgcgttttagtttttcgcgtgtttttggcgttttgcgttttcggattgggcggcgcgttttttcgatcggggcggtttcgtttagggttcggggtagcggcggcgtcggcggcgttcgagttttatcggaggagagacgcgcgagagaggagtttcgtcggtgcggcggcggtaatggagaggtaagaaggagttgttgtttttttggggcgagggcggggaagtgtgagagagatttgcgagaggttttagggttgttgttcggtttttcgtggggagggcgggaggaggggtcgaagtaaagaaaattcgtcgtttcggtagtttgtaagcgtgtcgcgaggtcggagcgtcgacggtggcgttaattatttttggtagtacgggaggaggtattgacgttaagtttcggattgggattcgagttttagttgttatttaaagagataggttgtagtcgcggttttcgggtttcgacgttagggggcggggtcgagagtcgagtttgtaagaggtgtagtttttcgattcgcggggataggtaggtagggagtttttaaagtaaaagcgagaaaaagggatagcgtttttatttttttattttattttttttttgggtttgtgttattttttttttttttttttttttttttttttttttttttttttttg

SEQ ID NO: 130 - DNA, 7618 bp, Artificial Sequence
  Synthetic
agaggtttagaaggtttttttatttggtatttagtaaggttaaggatttattttttaggatattttgtagtttagggatgtggttaggatttttggagatttttagtttgtgtttgggtgtgaatcgggagtgaaatgtgatttagaagtcgtagagaaagttttaagtagttgaggaggtgtacgtgagggtaggaagggtttggggagtgtgggaggttgggagtgagagttaatttgtttgttgatttgtagaagtttttggatttgttttttgaaagtaggatttagcgggagttggaaagaggaggtttttgtacggggttagtttgttttttttttttttatttaatgtggtggggtttttggggatttacgttttttagcggatagtagtatgttggaagggagtttggggatagatgaataataggattatagaaaattaagggggaaaaggttattagtatagaataaacgtgtgtttatagtgtacgtttagttgtgtaggttgtttatgggaagtcgttgtgagcgttggatcgttttagtttagtttaatggagagagggagggaagggttttgagtgtttttgagtggtatgtgagtaagtgaattttttattttttagataagatatagatagttgatagattttttttaaaaaagaaagaatcgaaaaaatgaatttaagtaatcgtggtagaagcgttttatttagaaatatagaggtaggtagaagtagaaagagttggcggggttgatagttgtgtttttcggtgggggtgggatgaggaattttttttggtagtggtttatgttttttgtagaagtggaggatattttaagttgtgtgttgagtagttttggtataaatgaagattaggaggaggagaagaggtcgggtaggggaaggggaggaagaagggggtattaggtttttttgaatttaaatcgtttaggcgtttatgtagatattttttttttttaaaaaaatggggatgcgggtggtggttatacgggtgtgtatatttattaaaatttttttgattgtatttttaaatttgtgtttttttttttaagtatatttattttttattttgaagaaggaggggagggaggttttttattttttatttggttatacgttcgaatttcgtgttttggcgatgtattgtttgtttaaaaattatatatattatgagggaaaatttttattaaaaattttggtcgggcgtagtggtttaagtttgtaattttaatatttggggaggtcgaggtagacggattatttgagtttaggagtttacgacgagtttgggtaatatatagtattttttttttataaaaaataaaaattagtcgggcgtggtggtcgcgtttgaagttttagttattcggagattgagatgggaggattattcgagtttaggaaatggaggttgtagtgagttttgatcgcgatattgtattttagtttgggcgatatagcggattattttaataataaatcgaaattttggttatttttaggtagtcgaatttgggatgttttttgtttttagtgtttgtaattttttgaatttttaaaataacgaatagagtttttttgcgatattaaatgtaatgtagtcgtcgaggaagcgtcggggatagatggggttggacgtcgtttttatttttttttttgtagtcgtttttaggtcgttaatttcgtttagtcggagttttagttttggagggtcgggcggcggcggtttttttttggagtggtatttttgttgttcgtggggttttttttttaggtaggaggtaggaggcggggtttggggtagttttcgtttttttttttttttttttttcgcggtttttttgttgcgtcgggttttttaggttttatttcgagttttcgtattcggtggggcgcgggtttcggtttagtttttttcggaagtgacggttgatttagagtttatttttggtttcggttttcgtattttttcgtttttttgtttgtggaattcgtagtttagaagtttggagtttttttgcgagtacgtaggggttttttagttttcggatcgtttcgtcgttcgttttgggtttgagtttgggggttcgggaaataattttggacgtttttggttggattgtgtttaaatcgtgtattttacgcgataggggattcgaggtttttgaggattttaaattgtcgtagtttttttttttttttttttttgtttttgtttttgtttagagaagtgcgtttttttagggttatttatacgcgtcgtgtcgagtttcgggttttagttttaggagtcgcgtttttacgggttagtgggttttgagagagtagggtttcgggcgttcggggagaggaggcggaggaggggtgaagtcgaggggtggtttttaacgaaaaagaagttgaagtattagttaagttattagggttaatgcgatgtattgtaagacggggattaatgtcgggtttcggggatcggcgtgggggagggggtagtaggtcgatcgtttttaagtcggtcgaggttttcgtttatttgggggaaatggattttcgcgatttaagaaataaatttaaattaaatgagcgaggttcggatgtgttgacgttgcggttacgcgcgcggagttggagtttcgagagcgttttaggaaaggcgtagcggcgatcgcgggagggggtgagaagtcgaggtagagaggttcggagaagtagaaagagatgtcgatcggtagagaaagagcgagatagatatatcgaggggtgagggggtaaaagggagtagtttcgtaattttcgtagaagaaaataggtcggcgcggttggggatgcgtttttgggttaggcgcggtaagattttaggaattagttagagttttaagatttaggcgtttatttgagtttttttgagttcggtaggaggtttaggtttattcgggagagagaggtgttagtttttattttgtgtagttttttcgttcggacgttatggggggagtgttttttgagcgtaggttttcgggcggtcggggtttcgtttttggcggtcgggagaggtcggtatcgtgatatttaggtatatatatattaatgtttcgggttcgttagggacgttgagttttaggtaacgaaaaacgaaggttttggagtcggttttgagtgtatttttttattgggtttcgtcgagaatgaaattttcggagtatcgttattgggaggttttggggggatattggggtgggcgagtttttttaaagattcggtttttattgggtttttttattttttttttttttcgttacgcggtggtggtggtggtggggggtgtaatagtttttttggtttttggggtattttttgtggtggggtgtgtttttttggttggatttattttttgttgggtgtaggagtagagtggggaggaaggaggtcggtggatttggtttttgggtagtttatttttgttttttttggggttatattgggtttatttgtaaaatgggtataggtgagatagagttggttaaggttttttttagtaggttaagggtttgttggggaggcgggttcgttttaaggttggtttttttttacgttttttgatagtgttatttttaggatatgaggaattttaggtaaatatgggggatttttgtttatataaatagttttagttatcgtaaatttataatgttagtattatttcgttagtacgatttttttagtttcgattgatagtttttttagaatttgggttttataggtttgagttggtttagagtttttattttaattttagaaaaggttcgcgggatttagaaaattttaaaaataaaaggtattttatagtatggggttttttgaggtgtagtttgggggtataagggtattgttttttagatttttatgtggagagttcgttggaagagggagaggaggattgaggaagtggggagggtgcgagattttcggagttgatatttttttttatttatttagattaggggtgagttgggtttagtatttttattttttcgttttgttattgtcgttattagagaaatggggatagagtttttgttttttttatatacgtgcgtggtttatgtgacgtggagtcgtattgggattcgaatgaggatattttttcgttttttcgttatcgtgggaatatcgtggaataaggcgggtaaagggttgattagttttcgtttcggttcgaagaaagtttttaggggaggcggaagggttattcgttattttagagtaggcgcgggtagtggtttacggttttgggttcggatttgtgtttatttttggatatttaattttcgttattatgttgttgggagtaaggaggtataattgttcgtggtgggagtggagggtttaggtttttattagaatcggtagagtttcgacggggttaacggcggtgttgatagcgcgaattgcgttaggggcgaagtcgagttcgtggagcgtcgttgcgtataagtgcgcgggttagtagggttttggtgtttggatattttcgggtgcgcgcggcgtggttgttttggtgttttagtgagagtatgattgcgcgttaaatggaagtgggggcgttttttggttttttttatattcggggttaaaatcgttgggatcgatatattgtaggagttcgggtataggttgaggggatcggttcgtgcgttgagcgtttcggaggtgtttcgggttatggtgggtgttgagcgggaatacggaaggttgtgtatatagtagtaagtaatgtattcgcgttttcgttattatatcgcgtcgtttttattttcgttacgatgattatttcgtagatgggtaagacgggtttagagagatgaagtgatttttttaaagttatatagcgggttagtgaaggtatcgcgtacggtttaggaatttaattttttttggggagtggggagggaagggttcgattcgaaggggttaggcggtagagagtttcgcgtttttcgttacgggttaagacgtaaggcggttcgtcgtgtttttatttgcgcgtttcgagtttcgtttcgttttagttttcgggttaatatcgtttttttttagaagtttaaggaggttcgggcggattagacgtttttttttttcgtagttttatgttttgttcgtagtttgagtttatcgtttttttcggggtggcggtgcggagatcggacggttttattgtgtagatttcgaggggtaaggggtcgggtaggggaggggagcgagaagtggcgaggtgagaattgaggggcgaggttgaaggttgagttagatttttagacgttagagagacgttaggtgagtcgggcgggggcgggggcgagggcgggggagggggcggggtcggaatcgcggggagggggcggggcgttcgggagggtcgcgagttgggaaggtttcgggcggtcggagacggtgaagaggcggggtcgagtcgcgttcgcggggtttcggatatagcggagttggcgttattgtcggggtttcgttttatttaggtcgggtagtttcgcgggttcgggcgtcgttgtttttttttatttgttgggaagcgtaggtcgagtagggatgaatttggttttagggttttagcggggtaggtagggttcggattagaagttgcggtatcgattttttattgttcgatcgggcgacgcggggtttgggaggggcgggtcgggattagagcgtcgttgcgtttcggaggtcgcgtttttcgcggtttgtgtcgaggagttttttcgtaaagaaataaatacgttttgttttttcgatttcgattcgggtcggttagaagtagggagaattacgttcgggaaaggggggtagggaacgacgggggagtttcggtgattagggtagatgtacgcgcgcgcgggattttcgtgcgtcgcgaagagggacgagtagaggagtatcggaagaagataggcgaaggggatcgcggagtagcgtaggcggagtttcgggggtacggtcgaggttgcgttttaggagtgttcgttaggcgttttttcgggcggttggcgaaattcgaggaggtttatagttttggtttggggcgtcgtcgttttaggggtttttgcgtttttttttggcgtttgtttggatttttagagtttcgggattaaggcgtttatttttttgatggcggggcgtatttttcgcgtgggtcgggcgttgtagattaagtacgtcgagtgcgttaaggaagtgttcggaaattaaagataacgtattcgggattaagaggaggtcgttgcgcgtaattttttaaatattgtagcgaagtcggaattacgttcgcgtgggtgattcgggttggggagtcgggaggcggcgttaaggattttttagtttttgttcgttttttttgatatgttttcgacgcggtttgaggtttttttagcggaggcgacgtttcggcgttaggtgttttgggtgggggaattcggagggggtattacgattagtggagaaggggcggaggtcgggaaagttgcgtggaaaagaggaagggttaagattgaaattattttatagagattacgttaatgggtttaaatgcgaatattttagttaagtagaaaaaaatttaaaagtagagtgtaagttggattaaagtgagatttcgattgggcgtagataaatttgtaattaaaatagttttaggtagagatttagtcgttggggagtataaatttttaatgttttttggtagttattttttgaaatattttattatggaagtttttaaatataaaataaaatcgagggaggttttaagtgaattttcgtgtatttatttttattttatagtttttaaatcgtgattaatttagttttttttattggtttttttttatttgtattaatttatttttgttgttttattataaagtaaatatagatatatttttattgaatatataaatgtatttttattgataataattttttttaaaataataatacggtttaagttataatttttagtaaatttatatttaaaggtagttggtttttgatgttggaagtgtagacgtatttttatttgaattttattgtaatgtttagataataaaaagtattattatttttgtaataaaaaataaataaaaatttagttttatagttttagaacgtggcgagggtcgtttgttttttaggcggtgaggttggtttttcgattatttagttgttttcggcgttcgattttgtgtgttttttcgtgtatgatacgtagagtttagttttgtgaggatagggttgtttttttttcggtttatttatgaaggggtgtttattttttcgtttttttttagggtagttatttattgggtgattcgttttggggatttggggatcggcgagtttaggaggtttagcgaacgaattttcggtggttgggatttattttttagcgggattttggataggtatgtttattttggttattagaagggtagggtggtcgaagggtatgaagtatatgagtgtataggcggggaggaatagtagtagtattagcgtaggagtagttcgttggt

SEQ ID NO: 131 - DNA, 2194 bp, Artificial Sequence
  Synthetic
ttaaatttagtatttattattgggatatttttatttgtttttataggaagggaaaggggaaggtatattggagttagcgtttatagggtagaattagacgagttttattggaggtaaattgggaggtaggcgtgcgttgttcgtggtgttgaaagtttgatcggcgcgagttggagtcgttatcggttgtttcggggtttcgtcgggttttatttgtttcgcgttttggaagtagattttgtagatgggttggtggtgttggtgttggtgtttagcgcgttggtcgtcgtcgttattgttgttgttgcggttgttgtatatcgagcgcgtttcgggttggttttcggcgtttcgtcgttcgcgttcgtcgttcgcgtcggttttagatttttcggggtcgtttttcgttgttgtcgttttcgggaggcgtttcggatttttttcggagtcgtcggtcgtcgttattttttggtcggcgggttgtaggggtaggggcggaggcggtagttcgttcgtttttttgtatcgcggggttattttttttagcggttcgtttggtttcgcggcgcgttcgggggtttcgggggacgcgggtagcggcggtaggtagcgcggggtcgcggggatcggggtcggttttttcggcggcggcgtcggcggcggcggcgggggaggttgcgggggaggttcggcgttttcgtttttcgtttcgcgatatcgattgtcgtcgtggtcgtttttaaagtttatggttgtgtcgtcgtcgtttttttgtcggttcggttggcgggtcgggttttggttgtagggaaagagagcgcggagggggcgggagggagaggggaaaaggagggagggggttcggacgtttggggttagggggcgggacggggaggggatgcggaaggttttgtagttgcggcggcggtaggcgcggtcgttcggtggagtcgtcggttcggttttgatggaggcggcgtcgaattcggttgcgcgtgagagtcgcgtcgcggaagggggggtcggagaagcgagggggcgggagggaggagcggcgcggcgggggtgacggggcgcgggcgcggggtgggttgggggcgcggattagtgggacggagttcggggttcggtttcgagcgggcgggttggaagtgggggattttttagtcgtttttacgggtcggtttcgcgtttacgtcggtttcggggcggatgatttttttttaaacggcgtagcgttgcggttttcgtgagttgggaagtagggggtaggggagaggtcgcgggtttagaaatcgttattggatgggtcggtgggatgtggcgcgggtcgggtggggcgcgatagtttgagtcgagattcgcgtgggtttaagggtgcgcgaggcgggtgttttgggcgcgttcgaattggttgagtagtggagcgggaaagggcgcgggattcgggattgtaatcgttatttttaggttttcgtttttcgcgtttggagtttttaagggtatttttagggattttcgagagttttaaaatagaagtttttggaatttgtgttttttttttgtttgtttcgttttcgaatttttgtgttttttttattcgtttttttttgtagtgagtatttcgggttgttggtaaagattttggtgtttgggaggtcggagtttcgttttttgaaatggtttatattagtgaattttggcgttacgttttgtggtttataattattttcgtcgttgtcgtatgaggaagtaaatgatatcgttttttattttggaaaagtggttgtagtttttttcggattttagttttatttatttcgaagttaatttttcggtaattttattttgtaaaatttttgtgggatttatttttagggtagagttaatagttttttttttggaaaaaaaaaaaaattttttatttgtagggaattaggttgagaatcgtgtatatgtagtagtttttaaattcgtgtagtgtgagattataaagtatcggatttatatgcggtagtgtgtttattcgaatttttattgatgtgacgtttttagtttttgatataaagatatagagtagttatttttggtagcgttttatagtcgaagatttaggattagaaaggtaaaataatttgtttaaggttatagaattattaataaagaatttttgtgttttttgattagatttttt

SEQ ID NO: 132 - DNA, 1958 bp, Artificial Sequence
  Synthetic
atgttgggttacgttgaaaatttgtggttgagagttggattttcgttatcgttacgtattttgttaatgagatagggtaatttatggttattgagtttcgattagttaaggggtagagagttttcgttttttattgttttataaaagagatttagtaaagggattttattagtttttagtttttagtttgcgcgagggtgtaggaaggaaagtttaggattttcggagtagagtatagtagttgtagaggtaaggttagtatgtcgtttaattttaaattttagtgttattttatttttatttttttgacggttttaagaggggaaagtcggtatttagaggtgagttatttttggattgatttattttattttttttgcgtgttagttttaggcggtaggtaggttgtttttgttcgtagttttttggggttcgttgttttttttttttttgttggatgttgttttgtttttggttttttttattttgttttttttttttgtttttgagtattttagattgtttgtttgtttttttttggtcgttggggatattttggtttgatggagagggtttaagattttttcgtttttttcggatacgggaaaatattatttttttttttttattcgttatgttgagtggtagggagggtgtttcgtttttggtttttttttttatttttagtttttgaattttttcggattgatttatgttttttgttagttgagggaagcggcggattacgatttttttttgttttttagcgttaatttgaagcgggagttggttggggagtagtcgtatcgtcgcgttttgcgtgagtcgaggttgttcggttcgcgggcgttcgggacgcggggaaggtgggattttgtgcggggggtagagggttcgcggatatttcggggaaggggttggtcggaatcgttaggggcgcggtttttttagttaaggatcggttcgcggaggcgcgttaggagcgggagagggtggcgcgttcggggcgtaggaatttagcgtagtttaggttggaagtcggggcgttgggtattatagagttcgggaatggggcgcgcggagagcggtcgttcgaggacggcgttgcggcggttgtagttcgggatttattgttttatgttttttttttttagggtgtttggatatgttgagtttttagggttagtttatttttatcgtcgatcggtcgtagttgtattgcgtagttttttttattagcgagttcgaggagtttattattatttattacgattaggtttttatcgattgttagggcggcgattttttgaaggtgaggcgtttacggttagttaatttagtcggagggcggtacgggagggttggaaagggttggggcgttgtatttagcgtggggttgttgagcgtaggtagttagtggtattttttagatatttcgttggtgttcgagtcggagaggcgcgttcgaggatttttatacggcgagaatttcgtttttattcgaagatttcgatttatatatatttagtggggttggggtcggtgtttttggacgttgttatggtttaaatttttttttgggcgttttcgaggttttaggaatttttaaagatttgaattcgtagcgcggaattttgacgtaatttagggtcgaggaaaaatgattaaaatttaataagttcgagtgttggggtttattaagcgggtcgttttggttagaaggttcgttttatacgttttatatttgagttcgtttcgtttttgttggtttacgttcggtagagtcgtttcgagagtatagaggttgtttttagtgcgcgggtgattcgattttggagtagtaggggattggttgtcgagggggtatttagattgagataggtgggagggtaggataagatttttaagtttattgttttttcgattttagtcgttagattttataagtataatagggt

SEQ ID NO: 133 - DNA, 9660 bp, Artificial Sequence
  Synthetic
gattttgtttttttagtatttttgggttttatttttattttaattcgaaattgttttaaggtaatggtgaatatatgttttaatggtttatatatacgtaattatatgtatatttatttagtatatttattggaggtgttgtgttgtataaagagataaaatgtataatagtgaaagggattagaatgtagagtttatttttaggagagtagtcgtggttagaaaagagtgtttttttggattaacggagttttttggggcgcggttttatggggatgtttaagttttaaaggaagaggtttaaagtaggttatttttcgtgaatttttcggatttttaagtttttagttttttagtttttttttcgttttttcgttttttttttttttttttttaatagttgttattaattggaggatggtaggttcgttagattattatttttatttatcgtcgatgtttcggggttgttgcgtatagatgtaagagtttttcgggtttttggggttgaagttttaaaagttacgcgttttagtattgtttttatattttaaattgttagttagttttggtgttttttagtttttgtgttgtttggtgggtgatcggtggaggggtttttttttgggggaagaacgttttaggcggggtttttaattgggtgattgtggtgtagatcgtagttttagagattttttcggaaagaagcgtcgttgggtagtagtttttttttagcgtcgcgttttttttttgttacgatttgcgggcggaggggaagggaacgagtatttggttttggttggtagagttaagtcgaggagttttattaggttttgatattttgtagcgtggttgttagatggttttgcggggtggtattttggttatcgtgttcgaagttcgcgttggttagtggtgtgtgtggcgtaggattcgagggcgcgttcgcgtagtaggtgtcgtagtttgcgtcgtagggtgtttcgttgtagttttcgtagtaagagtagggcgatattgttgtattgtaggggttggggaaggtaggtgcgtcgggttcggggtttaggtagggtttgttatcgcgtattaggacgggtatagttattcggcgcggcgttagagggtggttagttagttttagcgatttgttttggcgttgttttttgtatttgtagcgtcggttttggaattagattttgatttgcgtggacgtgagttgtagcgcgttggttaggtgttcgcgttcgggcgttgataggtatcgttgttgtttgaagcgtcgttttagggttagtatttgcgtttgcgaaaagagtacgcgcggttttcgtcgttgtcgcgttttgggttgtttcgagcggttatttcgtacgttgtcgtcgttgttgttaacgtcgcgttttggtattttggtttcgtcgtcgaggggcgaggtcgcgtttaggtcgggttttaaaagtataggaagggatatattagcgtttagtttaaggtttatttgagcggtttttatttagtttttaagatttttttttttttggtagtttttattttgcgtaggtttttttttttttttattgttttcgaattttgttttaggacgaattttgttttaggattgtttttatgttttttttttttaagttttgttatttttatatgttattgttggaatataaattgtgataatttatggtttttcgtgtgtgcgttttttttttaaaggtagattttagataaagagttaaggtagttttatttgttttttagaatgtgtgtgtatgggtcgggcgtagtggtttatgtttgtaattttagtattttgggaggtcgagacgggcggattacgaggttaggagatcgagattattttggttaatacgatgaaatttcgtttttattaaaaatataaaaaattagtcgggcgaggtggtatgtgtttgtagttttagttattcgggaggttgaggtaggagaatggcgtgaattcgggaggcggagtttgtagtgagtcgagatcgtgttattgtattttagtttgggtgatagagcgagatttcgttttaaaaaataaataaataaaaaataaataaataaaaaaaaaattgtgtgtgtatggtaaattttcgttgtgaggaataagtgtgagtggaggtgtttattagattgaggagattttggattttttttggaagtagagaaagaaggagaagaaagaggaagaggaagaggaggaggaagaggaggaggagaagaagaagaaggaaaggaaggaaaaatagagagaagttcgggtgttaagaaggcgtaaattcggtgataggtttttaggaggtttttgtttgttttattttttttcgtagttttaagcgtattgtagggaatttggttgtaagtttaggagcgggagttttgagtgttttttatttttatttaatttttgtgtatatttttgtttttttgacgtagcgttttattaatattagggatgtattttagtttttttggatgtgatgttttgtaatttaaattaataatatatgatgaaaataaaatatttattggtttttgatttttggtttagaaattgaggttttttttgagagatatataaagttgaggtttatatttggttttttaaaaagtagttttttttttttttggggtttttttggtaagtaggtggtgtgggttttattttgaagttttttttttatttagggcgatttttatagttgagattgtgtttattaagatgtagcgtttttttttttttttattttttttttgtttgggtttaaatttcgttttattttaagggtggatagaagggaaatgttgagtttttatgggattttacgttaggagaaaattaggagtatggaaggggtgtaagtttatggtaagacgggataggattttaaatgagttagtgcgattggaattagatttttaatatttaaggtattggtataattttttggagggggatggagttgtttggtatgtaaagtttgaggatagaataattttttttagagacgtttgtgtttagataaattgaggttatttttatttgtaaagaggaaaaattttgaggagggtttgtttttttagcgtatcgaaaggtaataattgatttttagttaggttgtagagtaaggaggattttattttaaagttatagattatgtaggttttggggttgtatagtatcgtagggtgggttagtattttgcgtttttttgtttataattttggagtttttgagggtaaggtggttagttgcgatggaggaaggtatgaagtttatcgaaggtgttttttttgttcggatcgttatcgcggttttgggtttagtttttggagtcggtttcgggttcggtttcgggttttttttcggcggcgtttattgttgtcgtagaagagacgtttttaggatttggtttttagtgggagaaggaaggcgtcgtaaatattatcggttgcgaggcgattgtggtggtcgaaggaacgtgattttttgttagtttagcggagtcgaagtgatcgtcgagttggttgagtagcgtcgagggagtttagaattcgttgtagaaaatttattagttttttcgcggatatagtaggtaatgtttttaggtttttggagttagtgagtagtcggtttgtaggtcggtaaataatttcgatttagttttttggtttatttttttcgttcgcgttttgaatggaggcgttatggtatagtttggtagaggaggtttgttttggcgggcgttatttagggtttttgcgttgagaatgtcgagtgaggtgttgaggtttggagagaaaaaggtggcgtttttattttttggaatatcgggtttttttagttgaagttttcggatcgatataatataattttcggatcgacgtaatataatttttcgttagtttttttttttttagcgggttggatataggtgcgtgggtttcgtagttcggcgcgtacggttgcgtttttggatatttgcgataggattcggttagaatcgtgttttagggtagagaaaattttggtaatttttaacgttttttttgacgcggagaattttgcgttaacggggttttttatttcgggagatttaatcgttattcggtttttaattttcgttttttagaagaaaaatttatgaggaggaatgggggggtttttcgattagattagtaagaaagggtttagttaggagtgattttagcgttacgagggttttattgttttgatggttatacgtttttgtaaacggtttgggggtggagagatattaggtgatggtagggaagggcggtggtgattggtacggttaagatattgcggagggtttcgttgtggagatgagaaggtggaggttttgggtattgtggttgcgagtgtgagcgatgcgtttgtgggaattagaggagcggatttgggaaaagtgaatttttgagatttagggttttattttcgttaggggtgtgtgaagtatattgggtgttaggagaggatgggaatgggattcgagaggtttttttttggatttttcgagagaaaatggagagagtttcgaatttaggagataggaggcgtattttttttatgtatttatggattacgttttcgtttttatatttttttcgtaggaggtaagatttgagcgtttatttacgtggtttttttattcgtttcgcgtttatttttaagattgttttatagggatttttaggaggtttcgaattatttagttttttgttatcgtcgtcgttattagcgttgtgaatttttgattttcgcggttttgcgtttatttttaggtattgaaagtttttcgggttttttcgtattcgcggatgtggcgaagtcgcggggtagtttcgttcgcgttttagtcgtaggatgtttttgatcgagaagggggtggaggtgacggggtttagtagtatttcgaaggcggatggggcggggtcgaggaggttcgggtgaggagcggtattttgaatttttcgttttgtcgttgtaggtttcgtagatagatttaagttttgggatagacgtttagcgttttagatagcgtttttttttgggttatgttggtaggttcgggtttagggtcgggtgacgagatcgtagttttttattggttttcgtagaaattacgtgttagttttgtattttttttttttagcgtttttttttcgcgttcgtcgaattttttttagtttttagtcgtttttttgggaggtttggttcgtattgacggtttttaaagtaaagaaaacgtggagagatagtgttgcgcgcggtttggattttcgttaatacggtattagcgggagttttcgaggttaatttagggtttacggggatttttttagtggatatgcggggattggaggtggagtttttgttgtgtaattcgattcggtttatttagtttggttttagtttaattttgtatttttatggcggaggaaagttaagatagagatagttgggattcgcggaatttgaggcgcgcgttgggattttacgtgcgtagaggtcggatcgtagagtagaagtgagttttgatttattagggtttatattgagatggaaatttttttttttgaatagaatttaggtttgcggagtttcgattcgttattttttaggagtagttttttagttttaggatcgaggaatgggtgtgggggttatatgtggggagttttcgattttttaaagggtttcgcgaatggtgggggagattggaaggtaatttttgcggttcgcgtaaagtggttagaggcgggagtgggggtgggggggggtgggttttcgtcgtttttggcgtttttacgtttttttttttttttttttttattttttttaaagcggattgtttggagcgtgttagtttgtgtgcgcgtataaatgcggcgttcgtcgaagggttgggaagatttgattcgtattagatttttagaaaaggtttttggttacggcgggtatcgagtaaatggcgtagtgtggtataaaggttattaatacggagatagcgttaatagatttagtttttgttgttattatttttggattagggaaacgtgatttggtgttagttgttttagtattgaatcgaagagggtttaaaaattgtgttttaattataatgggattttattagagtttttttggggattttagattaaatgtttttttttgttttttttttttttttttttttttttttttttttttttttttttttttttttttttttttttttttttttttttttttttttttttttttggttatagggtttttttttgttttttaggttggaatgtagtggtcggattatggttcgttgtagtttcgattcggatttagtgatttttttattttagttttttgagtagttgggattatagatgtagttattaagtttggttatttatttatttatttattgtttgtagagtcggggtttcgttatgttgtttaggttggttttgaatttttgggtttaagtaatgtgtttatttcggttttttaaagtgttgggattataggtgtgagttatcgcgtaggttttaaatgttttttaaatttatttacgtgaatttaatgttataatagttgtttgaatatgattattttttgaaatttagataaatttaatttatttttttaggttaatgggtgtaaaattgtatataaatatttatgtaaaattttagtatatttgattatttagttataaagtaatgattttttatatatggtaatatttaatttttatattaaagttatatttaaatatttttagttgttgtttttttttttttttttttttttttttttttttttttttttttttttttttttttttttttttttttttttttttttttttttttttttttttttttttttttttttttttttttttttttttttttttttttttttttttttgtttagcgaggagtttagattagagaggtggtttagatagtttattaaatggtaagattagattagtttttaaattaagataatttattttgtttttttttttttttgaaatatatattttttgtttttagagattttaaaattttttgatgatttatatatttatttgaatttttgttttttgataaaagttattatgcgtattttggaaaaagtgaagaaaagttaaaagaaaatatttatttagagataatattttagtggatttttttgtttttatttttgtatatatgagtttatagggtatatatgtttgtatatgtttaaatttatattataagtatgtttagtttatttttagaattttttaaataaatataatttaaaataggaatttaatattttatgttattgatagatcgtagtagattgtgtttgtatttttagataatatttttttttattattgtaaataagattttattttgtgtttttaatttttatttttgttaatattttttgttatttttttaggatatatatgtagaagaaatattaattagttaaatattttaatgtttataagatatgttattttagggttttttagaaaaatagtattatcgtattttgttttcggaggtgtaggcgagtgtttggtttacgggtgtttataattattatcgggtatttataagagattttgaatttagaatattttcgcggtaggagtttcgtttcgtttttttaggggatattgacgattcgttgggttttcgttttttaagtgttggaggggggaagtagggttattcggggtagaaatttaacgttagaaattttttagcgttttattagttggtggaattttcgtggtagatagtcgcgttttatttaagtcgtagaagaaaagatattttgtgggggatttggcgtcggaagttttgagtttaaatcgtagttttaggtttttagttttgttttttttggtttgttgcgaggatgaaatgagatttttgtacgtgaaaatgttttttaatttttttcgaggggtggaaacgtttttattcgattagagttcgggtttgcggagtaatttattcgtcgttttttaagagtaggttttttagcgtagcgagtttttgttatggaaagggtcgtttcggtttaggatgcgcgttttcggcgtagatttggggataggggtttttgtcgcgttcgttttatttttgtagggagtaaggtcgtttttttgtagtatcgcgttacgcggcgggaatttgtagtgttttgttttttggttttagttttttcgttcgtttgggtttttttcggttatttttcggaggggtttggagttttttttttttttttttattttagcgttagaagtattgaaagtcgttcgttcgtcgagagtggaatcggtatttcgaatttcgtttatttcgggttaattggaggcgtattagcggttgttttgtagtgtaatagaggttcgagtgataggttatttgtttaagcgttggcgtttaggaaagtcggggaggaaattaggaagggaggcgatagtagaaaagttagagagtttagagatttttaggtaggttttcgtgtagttttaatagcgtggtgtcgggaggtgtgtgtgtgtgtgtgtgtgtgtgtgtgtgtgtgtgtgtgtgtgttttttatttaggaatggagatatcgtgattatttttgtttttaggtgagatggagacggtcgtgttaggggtgtgattgattcgttttgttttatttaggttcgtagagtgttttaagcgtttatcgtgggttagttttgtttaagttttgggaatattgagaaaatgagaggttttagtttttttattcgaatttgtatttttgtatagaagatagatttttaaataatttttatatattgtgttattttgtataattagacggaatttgtatattatatttattgattttttttttttgagacggagtttcgtttttttgtttaggttggagtgtagtggtatgatttcggtttattgtaattttcgttttttcggttttaagtaatttttttgttttagtttttcgagtagttgggattataggtgttcgttattatattcggttaatttttgtatttttagtagagatcgggttttattatgttggttaggttagtttcgaatttttgatttcgtgatttattcgtttcggttttttaaagtgttgggattataggcgtgaattatcgcgttcggttttgaatttttattttttcgtggttaataaaagttattattttttgttttttttatttttttgaaacgaagggattttttttagtaagtattatatttaataggtaatttttggtaatttcgcggtttagtgggttttgtgggtcgaggtgaaattagttttttttaatttaagtgtaggattttcgttagattagattagatttgggttttgggtttgttgttatagttgtgtttttaaggtacgtagtaggtttttaataaatatttattgaggataggtatttgttttgtttgattggggatagtttttaggatattttggggattgttttaaagggtgtttggggattattaatggtggtaatttttgttttgttacggttttttttgttggga

SEQ ID NO: 134 - DNA, 1451 bp, Artificial Sequence
  Synthetic
tttttttttaaaaagattatttttttttatattaacgattttattttggaggtagtcgttgagataattttaggtgtaaaataagttaattaaatatacgttatttaaatattgagattagttggagtttaggtttttagagtaaagtaagagtataaattaatttcgttgagttgttgttatttttgagataattcgattttattacggagatttttaggtggttgcgaacgttaacgtttagtaggaacgattaagcgatttatagtatttaggggattgtgtaaatgttcggttcgggcgattagtagggatagatttaatttttcgggcgtaaagttatttttagttcgattttcgttttttttgaaaatttatcgttcgttttttcgggagttgttttcgtaggagaatgttcgtttttagtcgcgttttaattttgttttgcgtggttttaaattttttttggtggattaataggaaaataaatttaaaggatagacgttttatttgtgagtttggagttgggttgtatttggtgaggcgtttggttcgtatttttttgagtcgtttgtaggtggttggggaatggaagcgtttttttgcgcgtttttttcgacggtaggttttgttttacgttttttattttaagttaaaagtaagggtaggagaggcgggggttttaaatttacgtttcggagtatagagagttggttaatttttagcggggtttggggcgtttatatttacggtagagttatttgtgagtttgtagagtagtaacgggatgcgcgtaatcgggatgcggggacgatcgttatagcggttatagcggtaggcggggcgtagtcgttcgtatttattaggataagatgggaggacgcgtatacggcggggttttcgagcgcggttcgtcgttttagttcgtcggtttttcgtttattcgttttgaggttcgggttttggttcgtattggttgtcgttttcgttagtcgggttgggttcgggaggagattgagtcgttgtgagttcggcgtttcgagtttggcgttgttcggttttcgtcggtttttttttttgggttgtgcgttgtgcgttgggagcggggtcgtagcgcgtttagttttcgagttttttgttttacgttttttgggcgtagaggcgacgttggtagtcgttttttaggagattttttcgtagtgtggacgggggttaggatttgtaggcgagtaagggtagagaggagaaatcgtataggtcggtgacgcgtttaatttataaagtttgaaagtgttgttaattttggggttattttatgttgggtatcgtttttttaattatttgtaaaaagtgatagtagtcgcgtttttttttaggaggcgatgtgatgtgtaatggtggaaaagacgtttaatgaaaaggtacggaggtttgtaggataggttgttggg

SEQ ID NO: 135 - DNA, 1242 bp, Artificial Sequence
  Synthetic
tcgaataaaaaatataaattttgataggtaaattaagatgtttttatgtattttatgttaaaatcgtattaatttttcgtaaaattttttatttgcgttcgttttattttttggttcgtattgtttttagtaatgaagatttttttgtttattattaatattaattagtttatattaaattttaggacgttaaggaaaaggaaaaattatttttttagtagcggttatttaagaaaaaaaaaaattgataataatgtggaattttttttaaaattagtttatatcgtgatataaattggttttttttgtgatttaaaattgattcgtagttgattagttagaaaatagttgttgaaaagtcggggttttggagttttcggattttgttcgttttttattatgtaaataaacgtattagatttaaagtttttttataattgttatgttaattttggagcgtaatttttttatttgtatttgaaggagttaaatattaggtaggaaagaaagtgttttttttgaaagtttgagaaaatgttttcgttcggggttgtttcgttatttgggtcgcgggttgggcgcgcggttttcgtttttagttttttggtagaggcgtcggaggaaggggcgtcgcgaagggtcgttattttgttggaaaagaatgtagaaatgtttttttaaggttgaatgagtattatttttatatttagggcgggggaggtcgggggacgtgggagcggcgcgttaggagcgaggcgtttttggtgatagcgcgtttcgagggttttttttttttttagagcgtatattaaaggtagatttttttgtaggttttacgagttttattcgcggtggaaaggggcgttttagattgtagggtgaggggtttcggtgtgtgtgtggtgtgtgtggtgtgtgtgtgtacgcgcgcgtacgtttttaaggaaaaatttattttttataagtgttgtggtttatttttaattggatttcgtggagtatttagatatagtcggtatagtgaaggttaatagtattagtatattttcgcgtattaattttggagtaattaaaaataaaaatagtgagttatggggttttgttaaataagggagaaaaaaggttttaattttttattagtttattttgggggaatttagagagtttatgtatttttttatttttttatgtaatttgtatataaagtatgtttatcgggagttacggggaaaattatttttg

SEQ ID NO: 136 - DNA, 1206 bp, Artificial Sequence
  Synthetic
gagagtagtatttatttttttttattatttttgaattgcgagataaagaattttaattttggggttagaatttaatttgtttttgagaatatcgaatattattaagtaacgttagtacggatataatagtagtgtgaggtttgtatatacgtagttgaaatggtaaatttaaaataggagatattgtgtgtacgtatagtgtgtgtatatagtattaagatattttttgtttttggagttttttttgttttgttaaattttacgttttttttggaaagggggagcgaattttttaagttgtgaggatattttttttttatttagttatattttttttcgggttagtttttggtgaaatttgggcgatgtggagttattgggggatgtgagtgagggcgtttttgtttattttaatttaggtttttgggagagtttttaggatcgttttgggatttagaggaggtttcgttttgggtcgggagttgaggtttttcggataaacgttaagtagtttttatttatcggtgtttaataggttttatataaagtgttaaagtgttttagtaaggttttattagtatttgggtcgtagttgttttttggtttttggggttcgcggagttttagatgtatttgtagtttttttcgagttttgtaagttatttttgttttttttttttcgtttatttttcggtttttttattttttttgttatttcggggaaggttacgtagggtgtaattcggacgcgttttcgggggaagttcgcgacgtagtagttatatttttagtttgtattggcgtttattttatgatttcgttttcgcggtcgcgggtagggagcgcgcgtttttatcggttgattagaagcgggagtcggaggagttttcgttcgtcggcggttagcgtatagttcggttaggggcgttttttttgtcgtcgtcggttttttgatcgttcgcggttcgcggttcgtagattatagttagcgttgtgttgggtaggtggcggtagcggggcgcggcgcgtttaggcgtacgggttttcgggtcgcggcgcgtttttcgttacgtcgtatattaaggttcggtcggtcggcgggcgtttttattagtagtttgaaattataatattatgttaaagaataaagttgtttttcggaaaaatatgtgttgtatatttgataaagataaattggattattta

SEQ ID NO: 137 - DNA, 2308 bp, Artificial Sequence
  Synthetic
ttagttttaaagtgtagtgtggtattagcgaatgtggtggatttcggtaggtttggggtaggggaattttcggtaatattttttagtatcgaaggtaggatttttcggttattttcgtcggttggaatttttttaagtattgttttgagtttatttttaaatgaaagggggaaaaatttaaattagattagaatttgaaatagttgaaatagttatcgatttttagtttgtttttaagtttggtagagattgtgaaagttttaattttttcgcgatgttagttacggttcgcggcggttacggagttattttagtatttcgcgatggatagtgattttcggcgtggcgcgagttttttgcggtttttacgcgggtttgggcggcgggtttttttacggtttcgtttagatattcggaggtatttgttaaagttgcggaggtggacgatcgggcgtttaagtacgaattttttcgtcgaggtacggttgtcgggtttcgttttgagggtttggggcggcgaggatcgtcggggaggagggcgcgcgagtcggaggttagagtcgggtcgggtagggcgcgtttaggagtaggggagacggcgattcgggtcgaggcgggacggggatacgttatttgttcgtggagcgcggggaaggggtgcgcggcggcggcggcggcggagtttggcgtagagggtagcggtagtagcgggagtatcggttcgttcggggtcggttagtcggggagatcggggagtagcggggtcgcgggcggcggggtcgaggcgagtagtatcgggggaattacgattttcgcgttgagtcggagtagttcggtcgagttcggtattatttagatttagggcggcggcggcggcggttttgcgtttatcggttagaggagaggttgtcgtcgtcggggttgttgttgttgtagttgtcgtcgtcgtcgttggaatttggcgttcgtttgtattttatattttcgcgttgacggcggcggcgggggcggcgggcgttcgagatgttgggtcggcggcggcgcggtttcggagcgaatttatttggtttcgcggcgcggtcgttcgtttcgtttttgtttcgcgcgggaagtttttttttttttcgttcgcgtcgtcgtcgttttcggattcggttttcgtttcggttttcgttcgttcgcgttttttttcgtcggatttcgtttcgttttcgcgtttttcggttttggtcggggtttttttttcgttgcgtttttggattcgggcggtcgcggaggacgtgggtgggagagggtcgagggcgcgttcggttgggttttcgtcgatgtcggttttttggtggtttttggttttttgggatttagcgtatcgcgagggaggtttcgggtttcgtatttaagggtaagcggaagtcgaggtggcggcgtcggagttaggtatgtttcgttgtttagcgcggggttgagggcgtagacggggatcgcgcgagtcgttttttatttggttttttcggagtttagggcgcggagggtattgggttgcggattcggcgtaggcggtttcggacgttgttaggcgcgggcggatttttgttgtataaatgggttaaataggttcgtaaggttttcgcgggttttttggttcggaagttgagtcgcgggtgtttggtttgaagtttcgcgttttatagtggttcgcgggtttcggcggtttttgttttacgttttttaaggtattaggtaggtggttttagggtttttgtttacgatattttttgggtggggtaggattatttttttgtgaattcgttttggatgtttttaggtatagtttgtattttttttttttttagtatacgcggtttttgacgttgtacgtcgagttttttgggatttaggacgaatcgggttttaaggattttcgtttcgttcggattatacgtaggtagcgtcggtcgagtcgagtttgggttcgtattggttacgcgtagttatcgttagacggcgttagttttttttcgttgtagattttgtttcggaggtcgttagttttttttttatttcgggtttatagttttagtgtagtttttggcgacgcgcgttgatagttgggtagagattttcgtgcgttgtaaattttttaggtcgttaaatttaggaaatttaagtatagttaatgttagcgcgagattagtgttgttagaggtatttgaagggtttttttggatgtaaaaggtagtattatgatttatgtatttaagaatttgtatgaaaaattttttttgatttgggtgggagagtaggtatt

SEQ ID NO: 138 - DNA, 2201 bp, Artificial Sequence
  Synthetic
tttttttttttgtatgtaatttcgggcgagggaaagtagaatttggggggagattgtaaaatttagagtaaggtggattttgggtagagtgggggagtttatatttgtaagttgcgtgtaatcgaaaggttggcgatttgtagtttgttagatttagatatggtgtaggtgagggtttggtaaatatttagtttaatttttgagatcgttagagataaggttaaattttaggtgaacgcggttttagagtggttgggatttttatttttagatgggaattttttagataagagtaaaataggagggattttttcgtcgttcgttttgcggtgtgtgttacggaggtcgttgggtgggggcgtttaaatgtggaagcgtttttttagtatgcgtggatattttgaggatttacggatatcgattttttttttcgggtttagacgttttaattttcgttttttttgaggtgagatttcgtttttttttagttaaagggatttcggagtcgtatttatttgatttggttgttagcgttcgtttttttcgagtttttagggtaaggggttgtggtgtattttttggggatggtttgtggatgtggaagagggaaatttttttagggattttagttatttggtaattggtgatttttatatgacgtgttggttttaggggttaattttgataagttgggacgttttttttttgattttagaatagaaggtattttgggttaaggacgtttataattcggttttttgtttcgcgtggtttatggggtttgttttgggtttgggaggtcggtggcgggagttttttttatagtgaggtgggtggggggcgttatacggcgttttagggagaggggagaggcggtttttgtttcgggaagagggaacggtggaggtcgtcgtttttttgggcgcggtttttgttgggggacggcggggatcgtagggtcgaggggtcgggcgcgcgcgggggagggatttaggttaggggtcgttcgtttcggggcggggtttttggagagcgcgtagaggggacgtttcgtgaatgtttgtcggtttgaaggatgtcgttgatttttgtttttttcgttaaggtcgggttaggcgagtaggggcgcggtttgagtttttgcggtgattttttaggtgtggaattaaaatagattttagagcgcgattcgcggggtttttggaaggagtcgtttagggttggagatttcgacgtaaagattcggaggttcgcggagttgtgggcgtagttggagatggcggaggttttagtagggagtgggggttcggggttcgtagaggtagttttattttcggcgttgggggtcgtatggttttcgtaggttgtggttaatcgaggatcgggtttcgtattttttagacgtttttgagtttttttttgttttaacgtttttttttcgggtagttttatagtgggcgattttttcgtatttcgtacgagtttatttttatggtggattaggggtcgggtttaggttttgcgttattcggttagagcgtttagcggttttattgggcggcggcgttacgcggacgtgggtgggaatagttttcggattcgtcgggagattttcgcggggaagagaagtcgagtagtttgggggcgtttcggtagggagggacgtgcggttgggaagagttggtaggtggtattcgattcgtagtttggtgagtaaggtgttaattgttcgtttaattttttgtttttgtagtaaaattgtaaagggagatatttagttcggtaggaaatttagagttaaattttgtaatttaattttgtaatttaattttaattaaaaataaaattatttttttttaatatgtgtatatatatagagatagtaagcgaattgatttgggaattttatatatatatatatatacgtatatgtatatatatatgtgtgtgtgtgtatatatatgtatgtatatacgtatagttgatttgggaataaatagttgtttattgtgtggaatttttgaatatttataaaagttatttaaagaaagtaaaggtcgggtacggcggtttatatttgtaattttagtattttgggaggtcgaggttggcggattatttgaggttaggagttttagattagtttggttaacgtggtgaaatattttttttattaaataaataaataaataaataaatag

SEQ ID NO: 139 - DNA, 3047 bp, Artificial Sequence
  Synthetic
gtatcggttttgtattaaggttttatttttttattttttgaatagttagtaaaatacgaagataaggatgggaggaaaggttttttttcggttttgacgttttgtttttttacgaatcgtttttagatgtacggattttttttaggtaaaaagaaaaatattattaaaagaaattagttatttttattttagtgtgtagataaagaatttggagttagatttttttttttatcggttggtttttagttgtggttgggtattttcgaaagatagatggagagggagttaagtttttagggttagggtagtagttggtcgggcggagttttgttaggagcgggtttgggagttttaggatgtttggggcgtgagcggagggagggagtaaagtcgcgtttagttttttttttgtcgttgcgcgtggttcgcgttcgggttcgtttgttttttttttagtttaaagttttattcgtagcgtttgggttggttttatttatttgcgttaggtggaggggaaggggaggaggtttaaaggtgtggagtaagggagggagaggagaaaggaggaagggagagattgggtattttaagggttagaggaggattatttgtggttatagttatcgttacgattttttgtcgcgtttacgtacgcgtggatggggttaggttttgggtgttatttgggaggagtatttatttataattcggatttgttttcgtttgtgtttttttttttcgtaatttttattttttattttcgatttcgggtttttgagttaggttttttaaggggaaaatatttttggtatcgtttgttttggtgaaattaggtttttgttttatttagtttagttttgaataataattattgggaggataggaagtaataaggtaaattaagaaagtgataaggaggtgggcgtagcggtttacgtttgtaattttagcgttttgggaggtcgaggcgggaggatcgtttgagtttaggagttcgagattagtttggttaatatggcgagattcgtttttattaaaaatataaaaattagtcgggcgtggcggtatgcgtttgtaattttagttatttggtaggttggggtgggaggagcgtttgagttcgggaggtagaggttgtatgaatcgagatttaaaaataaaaagaaggaaaagaaaagaaaaaagaaataaaaaagcgaggaagtaagaaaagtaagtaagtaagaaagaaaagaaaagaaaggaagaagtggttattgtttttgtgttgtcgttgtgttattttcggtatatttgggtttaatttttttattttttatttgtgtttttttttatttgtttacgtttagaaatgtatttaagtgaataatagtttgggatgtaggtatattatttaatttatattgatgtttagtatagttatttgtaatatttaaaaaattgtaagttatttgttttataatagtcgattgattgtaaattatggtaattttttttttttgatacgtagttttgttttgtcgtttaggttggagtgtagtggcgcgattatcgtttttttggtttaagtaattttttgttttagtttttcgagtagttgggattataggtgtttgttatcgcgtttggttaatttttttgtatttttagtatagacggggttttattattttggttaggttggttttgaatttttgatttcgtaatgtattcgtttcggttttttaaagtgttgggattataggtgtgagttacgcgttcgattaattattttggtcgattttaatgggtacgttttaagttattttatcggagaatattattaaaatgaaatgtttattatatattaaatggggggaaagttgtttatagaatagtttttggtgaaaagagaaaatacgtagatataatttggatggcggagattgaagagtgattatttttaggtggttggatgttgggtataggttggtcgtaatgttatttttgtaaggttaggagaaaaacgttattttaaatatacggaatttgtttttttttaaatttattttttttttcgtttttttagaggttgttaggtttaagaagcggttcggagttgtaggaagggcgtcggcgttattggttttaagagttcggaacgcgcgcgtcgtaggagtgtcggttgcggggtcgggttgagattggcgggattttcggtttttgtcggggtgcggaaggtggatgttacgggtaaaggggcggggtttgcggtttttagatttagaggcgggttggggacgtgagtcggcgtttatgtgttttgtattttttttcgttcggtgttttttttaaggtacgttttttaaagtgtgttgaattcgggaatcgatcgaaaattttaaggttaattaaatgttttttgatgtagagtttcgattaggttcgaaaggttttaaatagtttttttagattttcgagggttttcgtcgcggtaattttaggcgtttttttttcgagaagtttttttgaggtttttatagagggcgggaggggttgcgttggggttttcgtttttagtgtttttgattggtggggagggatggttttagtgtttgagagtagagattagattgatttttatttgagattagaaaacgaagaggaagcggaattcgaggtggttgcggttttttcgggatataatcgggagaggaagttttggttatttgtcggtttcgtttgggggtatttgttcgaggatttttgggagtcgttatcgtattttttagaatttgtcgtgttgttttttcgtttttaaaattaataggatatttattttgtgattttttttttttttatcgtttagttggaggttgttggggattaggagaaaaaaaatgtttttttcgagttttttaatttaaatgtttattagttttgtatattattcggtttttttaaaagggattagaaaataaagatatgttaatgataaaggataattgagatttcgtttgttttgtatattttgtttatagaagttgtagtaataataatgtaatttt

SEQ ID NO: 140 - DNA, 7316 bp, Artificial Sequence
  Synthetic
acactattctacttttcactattaatccaacctactcccaaacatcacccctcctccgcaaaatactaaaaataaaaaaaacccaacgacacaaaaaacccaaaacttacccttctactaactcaataataactccgctacctatcataactaatatttccaaaaaaatcaaaacctcctcaacctacctcttccttatacacctttatttaatacaataaccctaaaaactctcacgattacgtttcttcttcatctattatctccacttttatttatttatttatatattttttaaactctatcacccaaactaaaatacaacaacgcgatctcgactcactacaacgtccgcctcctaaattcaaataattctcctacctcaacctcctaaataactaaaattacaaatatatacaccacgcccaattaatttttatatttttaataaaaataaaatttcaccatattaaccaaactaatcttaaactcctaacctcaaataatccacccacctcgacccccaaatatactaaaattacaaacataaaccaccgcgcccgaccctactatctccacttttaaaaattcaaatccacaacctaactcactaaaatactcacaaaaattcataaactatctaaactacataaataatctacataaattcaaaaccactacaaacaccatatctcaaacaaacgtatcataatatcaaaactcctattaaacaacctacaaaacaacctaaaccccgtaaattatatcactaaaacatcaacaattaattttatccaataacaaaatctaaaataactaaaaaaaataaaactcaaataaacctaaaaaaatctctcatacgataataacaatttcatcataaataataataataaccatatattaaacacttaaacttaaatcatacgtctaacactatattaaacattttctttatattctattatttaatctttacaaaaatcctaaaatcaacatcatcccaatttcacaaaatataacataacgtctcaaaaaaaataaacaacttaccaaacattccacaaccaaaaaactataaaaccaacctatactcctaaaaactaactcctaactactatatataaaactaaacctaataaaaatatcaaaaataaaaattcacttctatcatcaccgacctccctccacacctttacaaaaaaaaaaactaaaaaacaaaaatttccgcaaaacaaaaaacccaaattactacctcctataatttatacactacaccccaaactataaaaataacccaaaaacaaaactataacccaaacgaaaacatataccccatctaaaaccaccaaaatcttaccaacttcctcaactaataaatcgattaattcacgaccacaaccccctaaaactaaaaaaaaaaaaaaccaaaacgtctccgcaaaccaacccaaaaaaaaatctacgaaaaaacccgaaaacctacaaacccctctacacccccaaccccaccgccatcctaaacctccaaaataacctaatccaacaaaatcctacataaaaaaaactaaaacccaaaaaaaaacaaaaccccgcccaaaatcaccgaccgaacctaaataaaacccgattctccaaaaaccctatctttaactatcttatccaaataaaatttttcaaaccatcaaatttccgtactccctaaaataaaacttcatctaaaaccaaaaaaaaactaataaaaaaaataacaaaaaaaaaaaatacctcgaaaccccgaacaaaataaacctaaaaaaaaaacgaatccccatattccctttcccgctcaaataataaaaataaattaaaaaaaacaaaaacctccccaccttcaaaataaaaccctaaaaaaccaaaacacctttactaaaaaatatccctcctcactcctacacaaattcctcaaaaacaccctcgaactccgaaaacgaaaaaaaaaaaacgacgccccaaaaacccctaaacccctaattcttcccgaccctaaaaacttttctccctcgattcccaaacgacgacgacgaataacgcgaaacaacaaacgcaaaaacgctaaaataaaaatatctctacaaatctaaaattccccttaaaaatctaaacaaaaactacgacaacgccgtcccctcccccgaaaacccgacgccgcgcgaccacaaaaaacctaaaaaaacgaacaaaacctcgcaacgcacccaacacaatccgcgcgacgaaacgaataaaaaatcgacgaaaacgcgaatcgaccgaaatatcccccaaactccgcgtcgcgatccccgctcgccctcccgcccgcccaccgaacaccccaaccgcgcaaaaaacgaaaaccacgcgcgaaaaaccgcgatccgtccgaaactaaccccaaacccgacaccgccccgcgaaccgaacgcccacacccgccaaacccacgcgaacacgcccccgcgacgcaccgcccccaacccgacctccgcccctacaaccgcgaacacgcgaaaaaactcctaactacccgcacctacacccgcgcgtcgacgacgccgaaaccccgctccccgcctacgcgtctatctcgtccgcatctccgcgataaatcgacgacgccctcgcccctaaacccaaaaccaacttctctcgccgccgcgactactacgcgcgtccccgcccaacccaacccaaccccgaacacgaccccaaccccacgcacgaccctaaccccgcgaatcccgcaccgactcgctcccgccccatttcgcctccgcgaaaacgacgccccctcctccccgcgactcccgctctccttcctcgccttcccgaccgcgctaaaaacccccaaccgccgtccgcgaccccccaccgcgacgcccgaaaacgacgaaatctctttattcgaacgacgaacacgaaaaaccacctcccacgatatcaccgcacccaccccgcgcccttcctccgcctcctaaaattcaccgaaaccaaataacgacgaatacctttttaaaaatacgcgaccatacaattaataaatttttttaaaccgttttaaaaaaaaaaacgcgacgttaaaaacgaaaaaacgctcctaactataaactactcctaccgcttcgctccgcgctctcctaccgctccgctccgaatctcccgcgctcctctccccgactcgaccgaacgcgctaccccgacgccgccacccaaaaccgaaccgcgccgaacgccgaaataaaaatactaaaacaccgactaaaactactcacaaataccgcccgcctaccccgcaaccgaccgccactttccgaattaaaacgaactccgaacgcgacgaccgaaaactaaaacgactcgaatctaaacaaaaaaaaatacgaaccccaactaaatcctaattttatactacctatttatatacgaaacccaaccccgaaaaaaaacttaaaattataacgaatccctaacgctaacgtccgaactacgaaaacaccgatcaaaaaataaccccataaaatctctaaccaacgaaactcgaattaaaaccctaaaaactaactcaaaactcctaccctccaatcaatatcgcttatcccctaaaaaaaaacccgtaaacttctaacaaaacccgcgccataaacctcttatttctacgccctataacaatctaaaccgtctttctctaaaaaaaaaatttcttactaaaaataaaaacgacgccaaataacctaaaaaataaaaaaccaaattaaaccctactaactaaaaaccctcaaccttaaaactcctctaaaaaaataatcaattaccctactaaaaactcacatccaaaaaacaataactaaaaaacaaaaacgaacgatcaaaaaaaaaaaataaaaaaaaaaacaaaaacgaaaaaaaaaattcgaaaaataacttctatattctccccgatataaaaaaacccaaacaaaaaaaaaaaaaaacaacccgatttcctccaactctaaaacttataaatactccatccgtatatatcaaataaacacaaattccaataaatatcctccgacacctaaaaaaaaaaactaatcactaccttccaaaaccttaatatccgataaaaaaacaaaaccccgaaaccctattacaaaactaaaaaaaacaaccgtctataaatacgctcaaaaaacgataaaatccgaatcgaaaacaacttttaaaaacctcgaaaaacaatttttattaataaaaaaaaaaataacattataaattcaaaatcaaaaaaataacccatactctaaaatataaaaaaaacttattcgaaaaaaaatataaaaactaaataacctaaaatatacgtatatattacgaaaaaaaaaactaaaaaataaaaataaaatacgttaaataaaattttattaaatcgacaataacaaaccaaacgcaataactcatacctataatcccaacactttataaaactaaaaccaaaaaatcgcttaaaaccaaaaattcaaaaccaacctaaataacataacgaaaccccatctttacaaaaaaataaataaataaatcaacaataactcgtaacaaacttataaaaaaaaaaaaataaaaactattcctaaaacactaaaaactaactaaatactcaaaaattccaacgctaaacccaattaaccacgaaactaacaccccctccaatcccttaccaactttttctttataaaactcacttaaaacaaacccaactcacccaacttaaaaaaaaaccgcgcaaaccactaaacttattaaccccctaaaaaaaaaacttacgattccataaatattttatttatttattttattttaaaataaaatcccactctatcgcccaaactaaaatacaataacacaatctcgactccctacaacgtccgtctcccaaaatcaaaaaattctcgtacctcaacttctcaaataactaaaattacaaacacgcgccaccatacatacctaatttttatattcttaataaaaataaatttcgccatattaaccaaactaatctcgaacttctaacctcaaataatccacctacccctacctcctaaaatactaaaattacaaacgtaaaccaccgcacccaacccccaccataaatttttaaaactctacataataaacaaatctcctaattcctctcttccccaactcttcctacccccaaaaaacctaaataaaaaatactcacaaaaaacatatataaaaaactcaaacaaaaatctaaactttaaaaaccccatacactattctctcatctcctaaataaaaaaatcaaaacccctcttcccccaccaaataaaatcaccaaattctaacgaactttctaaatccctaaccaaaataaataaaacccctctcaattactatcatcaatcacaaactccgcccccattatacctacaacctaaccctaaaaaacaacccatcccctctattactattttataatcttcacaaacttccctctctatcctcttccttttctcacaaatcgaaattctaactccaaccccatcaataaccaactatataacttcaaacaaattacttaacctctcaaacttcaaccctctcatcttaaaaataaaactattaacccctatctctaaaaaataatataaaacttactaaaataatataaacgacacacgtaattaaaacctaaaccctaataaacacctcttaaaccccacctatcctaactaattaacacctaaaacatttaaatctacccccaccctcctttatccctactttcctacgtctataaaatttaacccatctcccccattaaataacaaacaaatcacaatccgaaaaataaccactttattataataacacacaacaaatactcaaaaattactaactaaaactaaactcacacaacccactcgacatctacgtctctaaaataattctataaaaacctctaactccctaccccgacatcaaccttaaaaaatttttactaaattttaaaaactcatattaaaaactaaacacaataactcacacctataattccaacactttaaaaaaccaaaacaaacaaatcactaaaatcaaaaattcaaaaccaacctaaccaacgtaataaaacccccatctctactaaaaatacaaaatcaactaaatataataacgcacgcctataatcccaactacttaaaaaactaaaacaaaaaaatcacttaaacctaaaaaaaaaaaattacaataaaccaaaattacaccactacaatccaacctacgcaacaaaacaaaactctatctcaaaaaaaaaaaattaatttaattctatatacttttcacattctcctattttattatcttacctatcttaccaataaaatcaataacttcctcatattcctatttctcaaaaaaaacaacactaaattcaaaacccaaccctaaaccgaaaattatcccatataacattctaactacctaactttaaaacaatctaataaccgttccacacttcattttcctcatctataaaataaaatcgtcgtaatacctacctaactaaaatctttaaaaattaaatatataaacgtatacaaaacgtcgaatacaacttctaacgaacgaataattaataacaatcgtaaaactaacgtcctcaaaccgcctcccgaatacactccatcactatactaaaaactccttctcctcccactatactccaatttccaaattccctccccgcaaaatattccaattccttcgttaaattctcacaacaatccaaacctctctaaactatatctctaaaaaaccctacttaaatctcaaattcctataaatcaaacataacctcaaaaaataaaacccttccaaccaaacacccacaactctaacatcataaaaaaaaccaaataccccctcacatataaaactaaaaaaaaacccataaattatcatacccaaatccaacatatctttttattctaaaaaaaaaaaaaaactattaaaatccttctaaaaactttataaatcataaatacttatcgaaaccatttccaaacgcctcaacgaaatcttactacaacaaaaacaaaaaaaacgaaaattttaaaatcaaaaaatataaatttcgaaatctcccctatacttctcaactcctaaacaaattattaccttctttcgacctcaatcttatcaataaaataaaaataataaccctcattaacaatattaaaccaaaacaattatctcacgcctataatcccaatactttaaaaaaccca

SEQ ID NO: 141 - DNA, 1587 bp, Artificial Sequence
  Synthetic
aaataaattataaataaaaacaattaaaaaccacgcttcatctccctccttaaaaactctaaaacgaaactttattcctaccaaaaattcaaattacattataatcatctcaattccaaaatattaaataacaaaaatatctacccatccttcactttccttaaaaaaaattactcttcgaattttatatacgattacaattttccaatatataaaactaaaaaaacaaaacactcaacgatatacatccctacacctaaataatcaaaaataataaacaactaaactaattatccttaattaacaaaatcaaaaccattaaaatactcactaatttaacaaataaatacccttaaacgtctatcatttataactcctaaaaactttaatttccacaaaaaacaaaataaaaaaaaccttctacttttaacaataaaaaaatcgttctccctcccctctccacccgaatcaactcttccaaccgctccctcctacatcacgaacacacgctacaaaaaaacgcatttacaacccgaaacatccccaaacctcctctccaaaattccccacctcctatacataaaaaaaactaaaaaaaaccctcacttcctttccaaacttcacaaacaaaaaaaaaaactataacaaactttcacctccgttcccaaaaacgaatataaaaaaatccgaaaaaacacgtcctacgaataaaaattaaaccgaaatctaaacaaaatacacgatccccgcaaactacgattaataaaaaaaatactaaaacgtttacgaaaaatataaaccataattatctcgccttcctcccctccctaccaactatatttcttaaaaaaatcgccgattcgattcacgcacacatttttataaaacacgaacaaaaccataaataattaccttcattattccgtcgaccacgaaaaaaactcgaactaaacgaaaaacaaatcccaaaaatcgtaacccctaaccgtataaaccgaatctacgactacaaaacgcgatcccgactacaacaaaacctaaaacaatacccgaaacgacgacgaatacacgtaacgaactaaattacaaaccgaccctctcgcgacgaaaactcgcgacctaacgaattacatcaacaaaaaaacactaaaactactcccccaaaccgcccccaaataataaaatctctcccaacccgaaaattcgaaaccaacgcccaaacccgaacctcactcactactcactcccgaaatacaaaacaaaaataccaatattacaaacaaataacacgattacccccgaatcaaccactataaatacgtatccgaatataaaaatacccgtataacatttatataaaaacgtcaaaaaaaaaaaaataaacaaatcaaaaatcaaatataattaccattccgtcatcactaactcctacccacctccctactatccccaaaataactttactacatactaaaaaaaccacgacacaatcctacccaaaaatatacatatatcccccgcgactactttaaatatacttttacaataatcaaaaacatatacctaatttaccgatctctttcccaa

SEQ ID NO: 142 - DNA, 3381 bp, Artificial Sequence
  Synthetic
aataaatcaataaacctttcactaccttccccttctctaaactataacaataaattaactaactcaaacttacccaacgaaatacccttcaaaacacactaaatactataataaaaactacatatacgctcaaaaaaaatataaataaacgctatttataattcacgaataaaaaaccttctcccaaaaacaacaaaaacaaatcctatctctaatataccctaacccccgacttaaaaacctcctaaaccaaaccaaaaccttccccgacaacgataatctccaaccacactcaactaccctaaaaaaacatttcctacttattcccttacccgactatatcctccacccgaaaaacctataccttcttcgcctacatatcctaccctaaaaaaactcccttaatctttcatcgctctccctataaatcttcacgccttcccgaaccaccgcgcccaaacaaaaacacgttctcgaccctcttcacaaaacgctcctcctcacaaaaatacccgaaatttttattctataccttcctaataactcctacaaatctaaaaaaacaaaaaacgcatctcactcctctaaatcccctcccctaacgcctaacgaaaacccaaactacatttataaaattcataactttttctctcctactcaaactaaacacattactaactcctactcgaataaaacccgactaattaaaataaaaaactccccgtaaaacgaaaaaatacgctatcaaatataacattcccgttttacgaaaacacacgatatcttacacgccaaaaaaaaatctaaaacgcaaaaaaccgtcgaacgaactacgaaattacttcgctatcacctccgcctacaaccacccttccgcacgcacttatatatacacccaaaccaacataaaaaacgccatcctaacttctaccgtaaacaaataaaaaaaaaaaaaaacgaaaaatattccattaattatctaaaaaaataaattacaccttcccctcccttacgaaaaatcaacttttcccaccccctcgaataaacactcgcatcctaaaaccgaaacctaaacccgaaaaccaaaaaaccccaattccaaaaacgtaaaactaaacgtacaacgaacgctcccaaacactaaaaaaaatactttacgatatcccgaatccctccaatctcgccaacgaaacgaacgtaaaaataccccgaccgaccaacgaccccgaatacaaaataacgaacccgacgacgcgcgtccccctccccctcctaacgacccgcacgtatcgcccgcgccgcgcccccacgaattacgcgcgaatcccgcaacgccgcgaccgaaccgaactacccgacccgcgaacacaacgccgaccgccgcatcccgtacgaaaccgcgacgcgatactacgctaaaataaaaaaacgcgacgacgaaaaaaaaacccgaacgcgatacgcgcgaaaataacgacgacgcgccgaacgaacccgacgcgaacgaacgaactacaaccgacgacgacgccaacaaatacgacccgcacccgccgccgccccgacgacctttaaaaactaaaccgaaacccgacgcgattacaaaattttcgtacgcgacccctctaacccgaaattacgactaaaacgcgcgccgcgcgaaccgaaaaactcgacgacgaaacgaaaacgaaacgacgcaccctctccgtatcccgctctacgcccttctacgcgccccgctccctataccgaaacaacgatccgaaaaacgaccgaaaaatacgcgcgaaaccgaaccgactacgaaacaaatcgaacaaaaaccgccaacgtacgtcaccccaaaatttacgaaataacaaaacgcgcgctctaaccacccgccgctctaaacgacaactaataacaacgcaaaaacgcgacgaaaacgaccgacgcgaaaaaaaccaaatacgaaacccgcgaacgacgctatacgcgcgaaacaaccgatcgaccgaaaacgcgaaaacctaatctaaaccgactaaaaacgaaaaatataacgaaaaaataaaaaacaatacgaataaacaacttcaaaaaatcattataaaaaaaaactaaaaaaaactccgcgaccaaattaacaaaacactctaacaaataactacgcgacccgcgcccgaaacgataactacgacaaaccccctaaatccccgcgcgacgcatcccaacctaaacgaaacgctcgaccgcgacgaaacgaacaaacctaacaaaacaaaaaaaaccccgactccgaaattactcttcgcacccgaaaatcaatcttaaccccaaaacgcgacgcacaaatccacgtaaatattttcaaattaaatttcaataaaaaaacttaaaataactaataaatttaaaaaaaaaaaaacacaataaaaaaaaacgataaaattaataaaccctaatatcgctcaaatccgcctctcttttctaaaaacaataaaaaaattcacttctatcaaacgtctattactctacactataccaacaaatacaaaaccaaaccgacataaaacacttctaaacaaccccgctatcaccaaaaaaaaaattctaactcccaaccatatttaaatttatataaacctacatatacccacgaaaatcaacctttataaaatcgtatataaaaaattttccttatatttaaaccgaaaactttctttttatactataaatataataaaatcgaatctaaacttaatttctacaaaaaaaaaattatcccgactttaaaaaattaatccttttactaaccgcaaatttaacgctcaaatcaccaaaccttctcaaaaaaacccttaataatattaaaacatcaaattacttacgattatatttaaaatatattttaaatatttatcaaacatcgctactaatacctaaaaaacctcgtaaaaacttatttttccttctaatttaaaaacatctaataaccgaaaaccgtaacgattccataaaatctaaccaaacacaactttcaaatacaacttccctctctctaaaaactacaacccacccaaactaaatttcaatacgatacgctttacttaaattacccactcacaatttcccactacgccgcaaacaatatatttcaactttaaaataccttattttaaaattccaaacaaaataatattaaaaaaatatctccttactaatcccatcaacttctattaaaaaaaaaaaatttataaaatttaaaaatactacgtataatatttaaactttcataaacattcaaatacttttaaaaccaaattcaatttaattataaatcgaaaaataaaaaaaacccacataaaaatatcctaaatcctcttaaatttattt

SEQ ID NO: 143 - DNA, 7398 bp, Artificial Sequence
  Synthetic
ctaaaaccgataaactctaaacacaatcacgaaaacatacgcactttctaactactctcctaccattatcccttaaattcccattttaataaaacgtacaccctaacgacgctccccaaaaaactaaatatatttactacactatacgtaaaaataaaaaatatacaattaactaaccctatactaatctaataaaactccataccaaccctaaaaaaaaacaattacataaatatatccacactcaccaaatactttttaaaaaaacactcgaaaataatactataacttaaaataactattataccgaacccgacatcttcccaaaaaaactatattattaaactaaatttcttaaatactaaacccccaaatccccaaatacgacgtaaacaaataacccaataaaaactaaactatccgataaacccaaatactaaaattcaaacaaaacataccctaacctaacgtaaaaaatacgaaatactattaataacaacaataactacatttctaaaacccgaactcccaaaccgacgaaaatatacacgcatctaaaatatctataattttacaattcccatatccacaaactcacttaattaaaaataattcaaaatatccaaaacataaaaaaaaaaatacctacttttcttaaaaaaaaaaaacttaatttcatctacttaaaaaaccacccaaacctaaaacattttccgcaaaaaaccccctaccccccgcctctccaaaataactaaaaaatctcaacactcctacacatttaaaatatttcaaaaaaaataaaaaaaaacaaataaacaacgaacgacctcaaacccaaaataaactatcaaacgctccccgaccacacattcaaaaaaccgaaatacaattataacgttacgacctactacttcgaaaataaaaatattattccatactataaattctcacataacccctaactctaaacaaaaaccgaaaatctaaaaaacgaaataacaaaaaaaacatacaaaaataaatttctaactttccaaaacgaataaaaaaaacgcctctctctcttataaataacaaacctaaaaaacccttcttaaaaataaaaacctattacttctcaaattctatatctaacccaaatccccaaatctaccccaacccctcgaccctacctaccttataaataatataatttaaaaataaaaaccgctaacctaaaaaaaaaactaaacctcaaattaaaacccaaaaaaaaaaaaaaaacccttaaaacaactccctttctactcactcactacctaactccttccttcacaccttccttcgaaaacgtctactcctaacaaaatctacttcctactctcaaaaaacccttattataaaaaaaaaaaaacgtcgcccgtccctaacttctctaacaaccgtattccatccccgccctataccccttctcccgaacaataccttctccaaaactcacccaaaaaaatacaacgataacccccgaaacgataatcgtaataaaaatattaactacaaaaataccctcgataaataaaaattaataacctctcgctaataccataaaactcgcatattcgccctacgcccctcgactcttaaacccacaaaccgaaatcctacctaccaaccgcgtacgctaccgtttaacccttacaaacgcaaaacgcgcgacgacgataacaaaaaactttatttaactacccaaatacaacctcctacaaaaaaaccctacgcccgaaaaaaaaaaaaaatctcttcccctctaaacgcccgccctcctcgccataacccgacctccacatccgcccacatctaaccgcaacgaaacgcccgaaaaaaaaaactaaaaccgcgtctctcgccgtcccctaaacgcgaaccaaacgaaaaaaaaaaaaacgctccgatcgtatacccaaaactatcccccaacgaccactcgaaccccaaccccccaaacctaaccttaacaaacgaacgaaacaaccaatacgaaacaaaaaaaccgatacgaatacgaaaacctaatccgcccgaaaaacgaaaacgaaacgaaaacgcaacgcgcgaaaaaaaaccgacgcccgccttcctcccccattcattcaactaaaccaaaaaacctaaaaactcctccgacgactaactctacactacaaaaacgcgaacgcgacgccccaaccaacgcgcaaaacccgaaccccgccgaaaacgcttcctcgccgctaccctccgcgcgacccgctacccaccaaccatcatatcgaaccccgcgatcaacgcgcaactaaataaaatcatttcgaacttcgaaaataaatactaaactaactactacgaccgcgaacgtactaaaaaaaaccctacgaataaacctaacgcgaaacgaaaatacgctaaaaaaaaacgaaaatacgctaaaaaaaaacgaaacccctaatccaaacgccctcccgctaaaaacgccgcgcgcccccgaccccgtacccgcgccgcctacgtaaaaaaccctattaaaaacacccgcgtaaaccctacgcgccctcacaaaaccctatactcgttctacgcactaccgcctaaatttccttccttttattattatttatatttaccaaacgacaacgacctcctcgaaaactcgcgaaactacctcgaaactctccaaaacgcacaatttcactctaaaaaatccatcgatcccctccctttaactctccccgacgactctcgaaccccgcttaaacccgacaacgaaataaaaaaatcgttcctcacctccgactaaataaacaaccgcgtcctactcgaataaacaaccctcccctcccccacgccaatttcgaaaccgccaaattatacaacccgtaaaccgaaaacaccgaacgaacacaacccaaatcgtaacaaaatctaaaataaaatatcccataacccccatccaaacctaaaaatatcctcatccgcctcccaaaatcgaaccgtaaaaaacaaaaaaaacctacgtacgaacaaaaaaaatattttaactctctcctatcttcgaaatttacaaaatatattaaaacttacgaaactactctatccaaacctaaatctaacgtccgcgtctctaaacctataaatacgtacgctttcctacgtcctcttaactaccgatactaaaactctacgtcctacgtccgcgaaaataaatacaacaaacgaaaaaaaaactcacacaataatctccaacgctctaaaacaaaacttctaaaaaacgaacctacctctaccgaaacctaaaacccccgcccctcgaaaaaactcctaaactaacttaaacaaaaccctctaataaaccgaaaaaaaaaaaaactatattaaaataaacaaactatccaaatatcaaaccaaactaaaaaataaccaacctaaaatcctccccgctccataaccaaaaccaaaactaacatctaaatatcctaaacccaactacccacacgacccacctaaaatcaaccctatctaaataaaaaaaacgaaacctcctaaaaaaccaaaactttaactaaacgccaaacaaaataccaataactattcttcaaaactaaacctaaaaaaaatataaaacgacgaaaaaacgaaaaaaaattataatccaataaccactaacgctatacaaaatataaactaaaaacatcgtaattactttatcaacttaataataaaaaccctttttcaaactctatccctttacatccctacttcccaaaaaaaaaaaaaatctaaatctacaaaactaaaaaaacttactattcccgcccccctcccccacaacacctcctcatctaaacatctttaaacacatactcatactaaaatctccctaaatccactatattccgttaaacctcctacaatccccgaataaatataacctccctacccctacctctttacaactcctccctacgaccgctcctccaaaaaccttccttatcccaaatatccaaataacacgacttaaccgatctaaccactttccaataaacccttataaaaaaaaaccctatattatacaaaactctcctcctacctacgaaatcgaaatctctactctcaattcctaacaaaaaatatcgaacccccaataaaatttctaaaaaaaaactctcgtatctcaacgaaaaccctataacgaaaaaaaaaaccaaaatttaaaattatattcgttatacaactatcaccatttacactataaaaattattaataccccttccttaaatctctaaatataactccacccttacccccatatacctccatctaaaactacttctacgactatctcccaaaccaattttataaccctataatttaatccaaaacaataaactcattaaaaccatcctaatacaacaattaacaatcctttaactctaaaaaaaaatttctcaatctcgaaaaataaaacctcaatctactaattccctatatttatcaatctcccccttatatatcctaaataattttactaaaaattctaatcttaaaaccatcaaataacccgaatcgataaacgtaaaaaaatatatatatacataaatacgcatatacataaaaactaacctaaaatataaaaaaataaccctccctaatacccaaaaaacctaaaatataattaaaaaatataaaaatatatatataaaaattaaacaaccttaaataaacaaacaaacgtaaaaaaaaaataattaaaaaaaataaaaaaaaaaatataattcaacccaaccaaaaataatataaacaaacaacttccgaatcaaaataaaaaaaaatcaccactaactaacaaaaaaaaaatcaatataaaaaaaacgaaccttaaaaaaaaataaaaattaaattacaaaaaaaaaaaaaaccttctaaccaacaacaaccaacaacaataaaaaaaactaaaataaactaactaaaaaaaaaactaaaacataaaaaaaaacctacctataaaaatcatataaaccaaactacgaaaaaccaaacatacccgccgaaaatacaactaatccacgaaaaacatctaaaataactaaaaataaacgcaaaaacaacgccgtaaaaacacaaatctctttcccgaaacgactcacctaatatcttaattcctacaaaataaaccgaaaaaataaaaaaaaaactaccaactccttacaacgctaaaataataaccccaaaattcctaaaaccaacgaatataaatacctatcaccatataaattactaaaaaacgaaaactccaaaaaccaccccaaaacaaaacgaactctaaaccacgacatctctaaaaacaattttccaatcgaacaaacgtctaaacctaaaatcctataacaaaaaccacaaaaccctaatcaaaatattctaaaaaacttaaaactaataacaatatacaaaataaacgacaaataacctaacataaaaaaaaaaacaaatacccaaaccgacacaacacacccgtaaaaaacaaataaacgaaaaaccgtccgtaaacctatatatatactcgaaattaaaattctaccaatatcccatatcctaaacacatctaccccctccctaaaaaacacctttctcttcaccttttccttccccgcctatccccttcacccaaaaccttccccatccctcccgtcttaaaaaccgaaaacaccataactttatattccatcaccgataaattacacaaaaattcaatccttccgctattcaaaccgaatccttcaaactcaaaattcccaaaaataaaaaaaatatcaatatcttccataaactcaaacttcccgcatcccgcctttactcccccttcaaaataaatctccgaacttcaaaattttaaaactctataaaaaccgccacgtaacaacaaaaaaaatattttatatttaactaataaaatttttaaaatctcattttctacttttccactatacaaacgaacctccaaacgactacaacacccgccactacccgtaataaaataacaaaaaaaataaccctttcctctttctcccttctaattaataaaaacacgaaactaacgaacgacatatactttcgtaaattcaaaccaaatctattatcgcaaacacgattacaactcgaatctttatataacaaaaacctttccaaataccaactattaacctactttactcaacgatatttactataaccaaatttacacaccgaaaaaaaatccaaaaatccttaatatttattaaaacaatctaacccaaaacccacgtactcaaatcccaaaactatataacatctaaacttctcccgaacccccactatcgcccgaaaaaaacccccaaatctatattctaaaaacaaaacaaactataaaacgaacttctaaataaaaaaaaccatataaatataccttcttatctaaaaatcccaacacctccgaaacgtaaaaactaacgcgctaacaaaattcaaatacctctttcctctctaaaaaaaaaaaccccgttacttaataataaatacaaacctatccctaattaataccaataaacctcttacgtaataaaccctacctttcaaccaaaactcaaaacatcctataaatactacctctctacaatttaaacttttataataaaaaacaaaaaccataaaaaataaaaacaaacctcttacacgaatcccacaaccactaacaacactaacttaatactctttaaattcaaaacccaaaaccaaacaaacccactatcccgactacgaattaattcatttaaaaaaaaacgaacacaacacccaaacaacaaatacactataatcaaccttacaacgaaactataaattctctaaactaaatatccgaaaaaaaacaaataaaaataaacgccaaaacacccctacaataactaaataactacaaactaaaaatactttctataaactataactatccaaaaaaattttaaataaaactaataaacaaaatttacatccaaaaaaacaatactttaaaaaaataaaaaatataacaatataaaaatacccaaaccgccctcactctaccattaaaaaactaaacgacttcgatttcttcaatttcttcgcctatttccctacctacaaaataaaattaaaaacaactctctttaaaaattactaaaaaactctaaaatacaacccataacgctaaacacgaatcctacctcctacgaatataataaatatcgcgactaatataacatctaaacgaaaaaaaaaaacatttacaaaaaacaaataaatttaaatacttaaatatacaaatctaaaaataaaaaatctcaaacaaaaacctatataataccactata

SEQ ID NO: 144 - DNA, 2396 bp, Artificial Sequence
  Synthetic
aaacacttcacattacttaaaaacatcttacactttttttttccctcaactcacaaaacaatttctttctactaatcgaattctcaaaacaaaaaaactacatacgtctctcgtttcttcactaattattctctaaaaaaaaaaaaataaaaaaaaaaaaaaaaaaaaacaacgaaaaaaaaaaaaacataaaaaaaaaaaaaaaaataaaaaaaaaaaaaaaaaaaaaaactaaaaaaacgcaaaaaccctaacttaccgataaacaaaaccgacgcaaccacaacgcgaaaccgcgacgcccactaatcctcgaaactaccaatcgacgtataatcctataaaaatttctcccgaatttatctaaaaatcacactaccgcctcctctccccaatcgcccaaaaaaacccgaaaaaacaaactcaaaaaaaaaaaaaccaaaaaaaaaaaaaaaaaaaaaaaaaaaaaaaacccgaaaaaaaaaacgcgacgcgaaaaaaaaaaaaacgcaaccgcgaaaccaataaccccgcttaaacgcgctactctccaaatacccccgaaactccaaccgcgcgaatcgcgcgctcccgccgctctacccctaaacttctaccgtaactccctttcaaaccaacgaatttattccttaaaaccaaaaactaaacctcgacacgaaaaaaaaatccgcgaaaaaacaaaaccacaacaaaacaaaaaaaacttcaaaaaacaaccttcccgaaacaccaactccgtatcgaaaatacaaaaaccaacaaataaaaaaacgccgcgttcccgaaacgcaactacgaacgacgaaaacaaacgcaaaaaaaaaaaacgaacgcccccgaaccccgaacccgaatccccgaacctaaaccgcaatcgctacgatactctactccgaattcgtatacgcgaactacgccgaacgctaaacaaaaaacttcgttttaccctaattacaaacaacgactaaaaacaaccgatccctaaaaaatatacgacgcgcgtaaatcctactcaccttaaacttaataacctacgtatcgacgaaatcgataaataaaccaaacgaaaaatacgcgcgccgtttaaaatatttaaaactacgaaaaaaacccgcaaaaaataaaaaaacgccacctaaaaaacccccaatccccaaatatacaccgaaaatccgctaaaacaaatacgctcgtccgatcaccctttccccctcttcccttcctcaaaaaaacgctactcgctaacgttaccccgcgatccgcgaaaataaaaacaccgaaaattacgatttaatctaaccgcaaaaacccctaaaatcactcccaacttcttcgccctcgacgaatcttactacgtaatctaaaaaaaacgaaaaaaaaaaaataacaaaaaaaaaaaacctaaatcgaacccgcgaaaaaacgactccactccgcgcgctcctcgaaaccaaaaataacctaaaaacttcgaaatcccttcctccgcacaccatcccccccgcgccaactttcctatttaactacatacaaattctaaaaaaataaaaaccaaatttaaaaaacccgcgaatatccaaaaaaaaaaatttacaaaaattcttttatttaatactccctacgactaaaacgaaataaccgacactacgtaaaatcgcaataaacgatccctcaaaaaaatactaaaaaaaacacgaaacgcgtccgaaaatactaaaacgccgaccactaaattcccaatcctacgacgaccccctcctcgttaaaaaataaaaattacaccgcgaaacgtcaaaaacgaaaaaacattttcataaaaattacacgaaaataccgcaaacaaaacgaaacgaaatacgtataacacgacgctcgacttcgaatcgcctaaccgctaaaaaacaaaaacttccctcccgccacgctcgccctctctaaccctaacgaaacgcttctaaaaccgaaaaaaatctcgtctccgacgaaacgcctaccgacacccaacttccctcccccgccctaacgataaaaacttaatttctccttttaatcgcgcttcgaaaactaaaacttatttccccacgtcgcccaataaacgccctctaaaaaaaactacctccttaacctcctctcgtccgcaactacctccacctaaacgccaaaaactctatcgaaccaaataaaaacttaaacaccccaaatttcgtctacaacctcaataccctctaaaatctcaaaaaatacgactatttctaacccttctaattccccacgtcctctccctttaccatttaataacgtatcaatttctaattaattaaataatcttttcaaaaacaaattatatcatcttaaaatcttttaaataattttat

SEQ ID NO: 145 - DNA, 3696 bp, Artificial Sequence
  Synthetic
taaaccgaatttaacatccttctatacaaaaattttccgacaaaacaaataaactactcttccactaaactataacttttaaactaactctaaaatacacccaaccttccaaaaatatctaacttaaccctcaaaaaaacaactccaaccccaaaaaccgaccgacaccacaacacctaaacaaaacactacaaaaaaaaaaacgaaatctctaataaaacttctcatttcctatcgctactaaaaaaaactaaactcgctccttctcacataacttaaaaaaaactataaacgaaaacttaccgactaaccaccaactcaaaaaccctaaaaacgacctccgaattctactccctattcttccctttaaccaatatcactcctaaacaaataacgaaaccaaacgcccttcacttcctcgaatcctcatatatatcataattcctcttttaaaaaaacataaatacaaaacgcaaaacaaaaaaaaaaactaaaaacaaattaccccatcacctcgtttcgtccctccccttcgttattataaaaaaacttaatcaaattccctttatcgcgccacacacacgcaaaataaataaaaaacaccctaaaacaacttatttctttcccgcctcacaaaaaacctttacgaaatcctcacaccatctccgaacgcaaaactttcgcattcaacttaaaaaactaaaccatttcaaaccaaaataaaaaacgccaaaataataccgaaataatcccaaaacaaaaaactaaaaaacaaaacaaactcaacgcacctaaacgtttacatttacacaaaaaaattaaccgcataattaataaaaactaccgactaaaaacgaaacgcccgtaccgacctcctcacctaaaacatctacgacctcaaattaatctccttcaaattaataaaatacgcccgaaaattattaaacaactaacaaaacaaaactccatcgcgaaaaatctacgcaaaatcgaacacctaaaccgaatcccaaatcacccgataattaataaacaacaccttacaataaataaaccactacgcgcactacttccacgactccatacccgacgactccgaaacgcgactaaaccgaaacgaacgacaaaaatacgaccgccgccgccgccgccgcgattcctccgcgccccgccgacgccaacaaccgccgaccctttccccgcgcgaaatcgaaaaaacaaaaaccgcgactaacgaatcgcgaacgccgcgctaaactcgactccgatcccgacccgaatacgccgcgacccgaccgccgctacaacgaatcccgcgcgctctccgtacgccccgaccgactcgacgacgactaccgcgcacaaacttccgactccaacgcccgacccgccactaaacatacccaacacgccgaccgatctcgctacgatccgcaaatccccaaacgcgcgaataaaaacgcgccgacgaccgaaactaaaatctataaccgaaaacgaaacgcgaaaaaaaaaccgacgaaaaaaaacgacgaccaaaaaaaaaatcccgcgcccccgcctacaaccttacgaaactcacgcaacccccgacgtcctaaaatcttctctcgaaacgacttcccgactttacgaaaaatataactaaatactataatacgataaaatcccccaaaaccccctaaacaacaaatactcaaaaaaacgacgaattaattaaaacacccaaactccccaaaacaataaaaaaaatacctaaaacatcaataaaacacgacgctaaaaattttaaaaatcgtcgccccctcgccccttacaactctatcccctccattctctataactaattcctcaacctattcctcctaattacccctaataatattttaacaccctcaaaataaataaaaatacgtatataaaaacgcctacgaaaaccgccccgatccctaaacctatttcctcgcgaaataattttcacttaaaactaatttacccctacactatcaaactcgaaactatttaccgttactattctaacccttttactaaccccacaaaaacctacttaaaaaaaacctataccacgatactaaactacgcatacgtcgacgactaacgaccgaatttaaaaacaaaacgcgttaaccctaaacaactcctaccgaactattctaaaatccttaataaaaattaaaacttaaccccaaaacttttacgcaatacacaaacaatacattcgtatttcttaaataaatcttttaaaaactctaaaccctaccccttttactcccactacacactacccctcaaaaccacaattaatatttaaattatttataacaaaatattaccacaataaattattactattcgataaaaatataatccttcgtcacccttttaaaatacattcacttaaaattaaaaaataaaccgaacgcgataactcacgcctataatcccaacactttaaaaaaccgaaacgaacgaatcacgaaatcaaaaaatcgaaaccatcctaactaacacgataaaaacccgtctctactaaaaatacaaaaaattaaccgaacgtaataacacgcacctataatcccaactactcgaaaaactaaaataaaaaaatcgcttaaacccataaaacgaaaattacgataaactaaaatccaaccactacactccaacctaaacgacaaaaccaaactctatctcaaaaaacaaacaaacaaacaaacaaaaaaataaaaatttaaataatttaattatattatatttctataacccaaattttttattacgaaccatactttataaacaaaccaaaatattatttttaaaaaaataatctattcaaaataataattacgtcctttttcgtttaactataacaaaaataacacactatttacactcaacgctaaacaataaatcaacctaaaaatttcctttcctcacacgcatccttccctaacaatttcctcttatattcaatacgaattccatcaataacacgacattttctaaaccataattttaacaaataaatcatactaatctctacatacctttaaaaacaaaatccaacacatttaaatcaccatttaacacaatctttcataaaaaaaaattaaaaaaaaatttcattaaacgaatcaaaatttaaactaaaactataaatttttttcaaataacatcaaatcacctttaaatattaaaaaatatttccaaaatccacattatatacactatcccaaaacgcaaatctaaatttaaaacccgaactatactactttctaacaacataaccttaaataaattctctacaattcaatttccataaatacgaaataaaaaatttttttttttttttataaaaaataaacgaaatctcgctctatcgctcaaactaaaatacaataacgcgatctcaactcactacaaactccacttcccgaattcacgccattctcctacctcaacctcccaaataactaaaactacaaacgccgaccgccacgcccgactaattttttttatatttttaatacaaacaaaatttcaccgtattaaccaaaataatctaaatcttctaacctcgtaatccgcccgcctcgacctcccaaaatactaaaattacaaacgttaaccgccgtacctaaccataaaaaataatttctaactaataaaattatt

SEQ ID NO: 146 - DNA, 4199 bp, Artificial Sequence
  Synthetic
tactaaaactaaaacaaaccacacctaaaaaacaatacccaaacatattaacgaaatttaaaataccaaaaaaattacaccacaattctacccaatatactacaaactatcaaaccgaaatactataccaactaaaaatacaacaactcccatcctctaaccctatttaattaaaaaacttcaacaaaacgaaacctaccaaacgttcgccgtcaaaatctaaaaaaacccgaacgaacaaaaaaaatacctaacccactccacaaaaacctatccaaaaataaaaaaatcaacgcccactaaaatcgattccgccctcgactcgcctacataaaacctaaccaacctcaatcatacccactccgacctaaaaaacccgcaaaatattctttttctcaactcccctatactaccttaaaacttaaaaaaacaaaaaaaaaaacatatctaaactacaaaaccaatatcttttaccgcctaaaaaaaaaaaaaataaaaaaaaaaaaaaaataaataaataaaaaaaaaaaaaaaaaaaaaaaaaaaaaaaaaaaaaaaaaaaaaaaaattctattaaaacccaactcctctaaaatctatataacctaatcttcaacgaaaaaccaatacgacctcataacacctttaccaaaaatcaacgattcccctacaatcaccatttaatttattactttctcgctcattctttctcataaaattatttcttcctcatcctaataaaacttttttctttaataataacaaaacttctatttcaatatttcccctaaaattaatactctttcaaaacaataaacccaaaaaaccatcccgtttaatatttctcaaaatcctcgcaactccaatataaacgcaaacatacaaaaatttcctactacacctacactttctacccatcccaaaaccacccctcacccccgaacctacaacaattccccttatttctctaaataaaaataaataatattaaaaatctaaaacaataaaaaataaaaaactaaaaaaacgcgctaaaataaactaatctatactaaatacgcgtattcttctacgaaattaaaaaatcgaaaacgaaaattctaaacttaccaaaacaattccaaccgataaacgtttaacaatcacttaaaaaaataaaaaaaacaacgaacttcaccgaacgaactacgataaataacataacgaacaacaacaacaaccaacaaaaaccctcgcaaaatatccaactactacactaccgcgaaaactcccacaacaccataactaattcgtacaactctacaacaacaaacgacttccgaaaaacacaaaatcgcgaaaaccgaacaacgaactactaaacatcccgcgaacgacgacaacaaaaacgacgacgataacaataacacccgacgaaaaaacaacaaccaaacccgcgcataatctcgaaaatttcaacaacatccaaaaactaaactcaaccccgaaacgaaaaaatcgtccgctaaaaaactacgccgaaaacgcgaaaaactactaccataaaaaaaaaactctaaaaaaccgaaaaacaaaaaaaaacgaaaatccaaaaacaaacgaataaaacccgaaaaaacaaaataaaaaaaaaatcaaaacgccccgcaacccgacaaccgcctctcgaactctaccgcccgcatccctctaacgtttaaaaaacaacaaatcctcaacccgcccgaaatcacgtaaaaaaaaacaatcgaactctaattaataaaacaaaatacaaatcccgaaaaaaaaaaatcgacgaaaaaatacaaaaatacaaaaaaaaaacgaccgcgaaaaccacaaataaactcgctcgccaaacgctaacccgaataaaactaaacgaaaataactcaaataaaaaactcgaacttcaaaataaactacccgcacactcatataccattcgcctcactctccgctccaaaacgccccctaccgaaaacgaaatccgaactaacgcccctcttccgcgcgtaaccccgaaccgcgaatacgaaccgcgactaaataacgtctctccgaactaaaaataataaaaacgaaaatatcaaaaaaacaacaacaacaaaacaaaaaaaaacgaatcgacgcgaaaaaaaacgtcctactaacgaccgacgctccaacgtacgaaaacgcgccgcctaaactataaaaaaatacaaactaaaaatatcgcgacgaaaaaaccaaaaacgtttctctaaaaatttacaaaaaaaaaaataaaaaacgataatattaaaaccgctcttaccgacctaaaaacaacaacaacatctcccacaaaaacgtacaaccccaaaactactcgccgaaacaactcaaccatcccgacaaacgctctccttccttctctcttctcccctctctcctcccaaaccccccgcaactccgacccaacccaaacgttcgcaaatttaaatccctctcctcatcacccgctcctctccaacccgtaacctattaatatatccacctaaaaaatacgatcaaatatatttaaaaaatcaaattaatcgaaacaaataatctaaaaaaaaaaaaaaaactcctctacatacgccgcgaataaattaccgaaaacatcgaccgaacaacgacgtccgaaaaaaaaaaaacgaactccatttattaacccaaacaataaccctacgttccttactcgaatctttaccgaataaccgataacctaaaacgacgaaaaaaaatctaactcgacaaaaatctctaactctacgcgtttctttcattctctccaacgaaaaaaacaaacgacataacgaaacccgccttccgtctactacattcttcaaacaattaaacacactctttaacctaataaaattttaatcgccaataacgaaaccaaaaactttcgaaaacaaaaataaaaaaaaacatctttcttccataaccgaaatcactattacaatctcaatattttaaataccccataaaaaaaaactttctttttaatatataattattcaataattcctatttttatttttatttatcttctctccgctctccttctctattcctctctattatccctcctccttcttctccccccgctttcaaaaacctccgaatcctccctttttctatttaaattctccttttatacccctctttctatatcccctaaatttaaaaaaacatttaataacatttaacaaacaattaatatccattcccaatcacttaaaaaaaacattcatatactttaaaaacgaaactatctatcctttacaaacaccaacaaaaaaacaaattatacccgaataatccttttaaatactttaacctccaacctcctcccacttccttactttttaacttctcctttaaaaaatataatcgtacaacacctcaatacctcaacgaaatctttttttttttcctatataaaatccatccctttatcttacatctccgcctccgtccgaaactatccctctcccctcccacctccaaaaatttctaaatctcaatatctctcactcctaacaattaaacaacaaatcccaacattctaatcgataacatctcgctcctcaccgacgaaaactccattaaaacaaatcaattaaaccaaacgttaaaaacatcaaaaaatcgacttctaaacaaaacaactaaattctttaaaaaaacaaaatacccattaaataaaactaccaactaatattacaaaacaaaaaattaaaaatttctttcgctacaaactttcaacaaaaaaaacaacataaatataaatcaaaatttaacaactctacaacaaaaaataaaaacatatcattttccataacaaaactaatataataactaatcaaacttataaaaataaatcatacttaaaactaaaaacaaaatccttaaaaatatttatacaataattataataataaaacaaaacctactaaaatttcaaaatttaactatataaataaaattttaaaaaacctcttaacaaaaaaaaactatttttaaattttctactaaataaatttttaacatactttctaataatatatactcttcctaaaacgttttaccaaaaataaattaaaactccaaaaaaattaattactaattataactaattaacaaatacgattacttccacaaaaatcctttaaattatt

SEQ ID NO: 147 - DNA, 5716 bp, Artificial Sequence
  Synthetic
tatatatatatccacgacgcgaaccgaaacactcaccatctcgccgaaaaaacgaaaccacttcgaaatcgaattaaaaatattaaaacttcgcttacttcctccgcgacatactaactcaaacatcaactaaaacaaaataaaaaattactaaaaaaaaatcttctcaaaattacgaaaatcgaacgacgacgacgacgacgactccccccgcccgccgacccgcccgccccctccccgactccccgaccccgaacgcaacgcgcatatatcctactataattataattatcaataatacattacgattaatcataaaaaaactctttaaaaaacgattaacaccgaaccaacgctattcaaacccgctcgccttaatcaattaattcgtaatttactacaaacccctatctctccgcgcgctaacccaataaaccgaccgcgaaactaactctacgcgccgcgccgcccgacactaaattaaccctctttacgaccgcccgaaactccgcgacccgaccgccgaaaaccgacctcctctccactacgaacccgacgccccgacaaacgccccccgctctctccctctcccctccctcttccctccctccttcgcccccccccacaatcccgaactaaaccgacccaaattaacataccctccccgaaattaaaccgccccgaataaaaaataaaaccgaacgacgaaaactaaaaacccgaactaccgacgcgctacgaacttcccaactcgaaaacgaaacgcgccgacccgcgaaaaaaaaaaatcccgcgacgaaatcaaaaaaaaaaacgtcccgtttcgtaaatatttcttcgtttctttaaaaactaccaaaaaacaacccacccccacccccattaaaaaaaaaaataaacgaataaaaaaaaaccaaccccacgccgatccgcctcccgcccacgactcgacctccctcccgcctaactcgacgctcctccgccccgaattctccatcccctaaatcccgaactcgcgaaaacgcgaaaaaaaaaatccaaaaaaaatttaaaaacctaataaatttataactaaaaaaaaaaacaattctcccgccaccccatcccctccccgccaaaaacactaatcctccaacttcttacttactccctcgctccctttctctacctcgaacattcccaaaacaattaaaactaaaattttcgaaaaaaacaaccccgaacccgtaaaataaaaaaacgacccgactccgcccaaaacccctcccgaacctcccccccgactaccccgattaactacaaaacgcgtcactccgcgaaaaaacgacgacaacgataacgccgcaaaaccccgaacgaacgaaaaacgaaaaccgacctaaactccgaattcaactccgaccgaaaaaaaaaaaaaaactacgactcacactaaacgcaacaacgaacaccgacctaaaaaaacacaacccgaacgaacccgacactaaactaaaccaatccctaaaacccctaaacaccccgccctaaaaaaaaaaccgcgccccaaccttccgaaacgaaataacgacgcaaaaccgaaaaacgaaataaaaaaaaaacgataaaaaaatattctatccgaaactacctaacgacgccgaaactcgcctccactttttaaacacttaccgaacgcgacgcccgaacttttcgctctatcaccatctccctcaaactaaaatacaacctccctaccctcgccccaaaacccaaaaccaaaaacccgatccctctaccgaactcaaacgcccgcgaacctaaaactcgatacgcgactcaacctaactctttcccctcgcgcctcgaaatctcctcgctccaaaattctcgaaatcttacgcccgaaaaactaaactaaactaaacttcgactaaataataaaaaaaaaaaaaaatacaaatacacaatccctaccctatctcacgatcaaaaaaaaaaaaaactcataaaaacgccccaattcgcgaacgcccttaaatctaaccgataccacgatcgattcgcgcgaaataaacaccgatctcccgaaccgacttcgtatactccgaacctatactacgctccgataaaaaacccttccctttctcttaacttcttaaatcttttcttttaaaatataaatatatatatataccgataaatatttctatttttcatctactaactttatttttttttattaatataacttctcccttctctctccctactcgtccgttttccaccttttctcttacttatcttctaccctcgctcactccctaacacacacattcacaccctctccgcctcgctctcccgttctcgccaaatcctaataaatacaataaattttaataaaaaaacaaacgaaactatcaataaaactaatattaaaaaataaaaataataatatttccataaataatacttcaaataccattatttctcactaaatatttaaaaaaatccctcgacaaaaataaatctacgcactcctaaaatataaacgactcgcatctcccaaaccccgaaaaacgtatacgaaaacacgtataaatcccgcacccgcctccatctcacaaacaaaaatcctcgcacacccaccaatccacactatcccaaatacacgaacgcgtttatatacacttacacactcctttcccgaaatatacccgtatttaaaaccgcccgcatccttctctaacaatcttaacttcttaaaaatccgaacctcccgaacaccgcatacaaccaaaaacctaacgaaactaacgatcccgcaaaacttttctcctaatacgcgccgcgtacgcaacaaaaaactcccgaaaataacttcacacgtatttattacttaatctacgtataatttaaaattaaatataaaaaaaaaaaaaattccccccccccaaaaaaacaccacaacaaacaaccaaacgaaataaacatacgaaaaacaactacgcgatacttaattaaaaataaacgaacaaaaacgaataaattaaaaaatccaaactttaataacttttttcgtttttttacgtcctcgacgacgtaaaaacgaacgtttcgatctacgcttaacgtcccccgctaatttcttatttattttctttccctcgataaccccgaaaaaaaaaataaaaaataaaaaaaaaaaaaataaaaaaaaaaaaataatcgaaataacgaaaaaaaaaaaattaatctttattattaataacaatacatcaattacgaaccattatctcgatcccaaattccgtaattcgctaatacgaacgctacaatatcaaaacgctaacgaaactccgcgtaccgcgatacaaaaaaatacatcaataaaaacaaaaacaaaataaaaattattctttatttttatacgaatcgaaaataaacgacgaacgaaaataaaataccactcctaaaatccctccacgcgccctactcaccccaaaaacctacgcgctctacgtacgataacaccctcctaactctaaataaaaataaaataaaaaaaaaactactaaaaaatccgaataacgaaatttaaacttacaaattccgccaaaaaacctaatacttaaaaactaatccgcaataacgaataaaaataacccgaaataaaacgacaactacgccgaaatcgcgacctcactaacgaaaaaaaaccgcacccgaaaaaaacgcttaacgaattttacaaactcccatcttaaaaaattaatactcgttcctactaatacgtccccttaattttcccaaatcctaaaatatccttaataaattaacgaaaccgaaaatctataccccaaaaccgcaaaccacaactaaaatcccaaccataaaatttccaaacttctttactcaacctctaatcctacgaaaaacaacgtaaaaaacgtcaaaactcgcgcattctaaaacctaattcttctaccctcaaaaatatcttattctaacccgatctaacctcgaatctaaaataaaacctaaaataaaaccttacctaaaccccaaaccctcttttccgttccgttaacgatacttaaccaccctaccccaaaaataaaaaacgtctccaaatactctccctacacccctatttcctcaatccccaactcaaaaccccgctaacccccgaccgaaactaaaaaaacaataataaaaatttcgttttcttaaacaaaacaaaaaacgacgaaaaaacgacaacgacgccccgaaaacgaaaaacgaaacacatcgataatatcaacattattaataaaaaaaaattcgaaacaaaaacccgaaaatcaaattcccaatccctcccctacccgaaaataaataaaacaaaacgaataaccaactatatatatatatacatatatatatattaataaaaaaatattcctaaatacacccgcattaaccacctaaaatttatttatattttccttatctaaaaatttacgaaaacaaaaaaaaaaaaaaaaattaaaataacttataccctctaacccttaaaactcttaactaaaattaaaattaaaattaaaattaaaattaaaaaaaaaatacgaaaaaaacaaaaaaaaaataacaaaaaaaaaaatcaaatttaaaaaactctccgtaaattcgattatttttccaaaccccgaaaccttacttccccgaaaatttcaaaaactcctttcctactaaactactatcactcaatcctaacctctcttaaaacactaaaacgacttcgaacccgaaatataaaacaaacttaaataaatacctcgcccctaaccaaatctacactatacgccctcgacgcactcgctctccaacaattcaataccaacaacaaaacgcaaatttttaaaaaaactcccgccgcccgccaccaaaaactatctccaaacgaacgccgaatacaacgccgtaaccgaacgccctaacgccgactcgaacgcgaaattcaacgataacaaacgaaaaataaacttaataaccacccgcgcgcgcccgaaccaaaaatcgcgtactatctacccgcgacaaaattcgtctttctccgcttaaaaaactattcctacaccgatattaaaaaaccgacttcgctaacgactacaaatacttacgattttaactttccgtccacaattaaacgtcttacacttaaattcactacgccccgcatacaacaatacctcgccaaaaaacctctaaatataaaattcctcccaaaaaaaatatcccgacgtaaaaaaaataaaaaaaactaatcgaatttataaaaaaaaaatctttcctccttttaattaaatccctttacgattttctaaacgcgaaaaaaaaattatcaccatataataaaatataactaattcttaaaacctaataataaaatattcaaaaaacccaacaaaacgcacaaattcccatctaccgctccaataaaaccttaattaaaatccaacgaataataattaattcctatatcttctattaaaaataaatataaaatctataaaatcctattctcgaaacgtatacatttaaaaaaaactaataatctaccaaaaaactcactttcattcataaataaaatcccctcaaacgacctccttataacattcctaaataaactaacttaacacatctcttacaacaaaattttatttaaaaaaaatttacgctaccacttttt

SEQ ID NO: 148 - DNA, 5474 bp, Artificial Sequence
  Synthetic
ttttcaaaaacaaaacccccattctcccttcatccacttcttacaatctaattaactccctcctccttcttctttaaacaacgaaaaaactctccaattatccaacttccacccctccaactacaacatcgtcatcctttaactcaacgaacgctaaacgaacgctccctccccgcccctcctacccctcctccctactccaaaccctcgacttctcatccgctaaacgatatcctacttcgctcgtccttactctcgccgctactaccgaaaccgaaacaaaaaaaacaacgaatcccgtaaccgtcccgaaaaccccgcgctcccgaccaaaaaacgaaaacgaccccgaaaaaaacgaaacaaaaacgaaaaaaaaaaaaaaaattttatactacgccgaaaaaaccgacgccctcttccgaatatcctacgaccccaacctctcctcacgctcgcgcaatctccgccgcaatctcaactacaactacaaaactaaaccgtacacccgaaaaaaacccccgaaaaaaacgacaaacttcgcaataccgcgacccaaccccaaccctaaataaataaatacctccgcaaccccgccgcccgccgtaaaatcgaaaacaaaaaaaaaaaaatacctacctaatctacgccccccgcctatcaacccttacctcgaaactctaaaacacccaactcgtcgactcctaacaccgcaacgaaataaactactaaacaaccccgaacgcctacaactactactaccatctctaatctacatacttccaactctaccaataacaacccccctactactcgcaatctaatcaacaacccctcgaatcctcgcatcttccctactacgcgctcctatcccaacgcgaccgccgaaatttccgaccttatcccaaacaaaacgataacgttccgaatcaatccttacctccgtacccccacgcacaaacaactccaaaaacaacgaccaacaacccttctaaaaacaccttactataatccgacaaaacaaccgatcaaaaccgctctaaaaaataatcgaaatacccatttcgactaaaatcccaactcgactccttactctataaccttaaaataattaattaacttctctaaacctcgaattcctcatcgatataataaacaaaacataaacaatctttttctcacaattaaaattatccacaaaaaacatttaaaaaaactaactcaaataaacatcaataactattaactaaaattcgattaccactattacattccttcctaacactaccaaaaacctaaaaattccctctcccaaatatttccatatcctatcaaatattcccccacctattaaaataactctacctacataattctaataaaaccaaaaaattaacgtcaaatccaaaaaacaccattattatctacattacttaaattacaaataataacaataaaatcaaaaaacaatattacccttaaacccacccacaatattaccaatacctatattactataactaactttactatttataacaacatcatccacatttcaaattctatacgaaaaaaatctacaactactcgactaatatcattactaataatattattaattttacttccaataaaaaaaactatttatataataattacaattacttctacaaaccctaaaaccctacttatattatataaactcatattactcctcataaaaccgttaaaaaaataacaaaaaacttccctcctactataaccaaaactctaacttcttatacctccctactaaccctactaaatactaaaactcaattcctataaaacaaaccttaactaccgactaataaacattcaacataaaaataataaatattattaaatatatatacacacgtacatacctacacacacacacacacacacacacacaaaaaaaaaaaaaaaaaaaaaaaaaaaaaaaaacctaatcttaactctctaaattaaatctataaaaactataaaaaaaataaacatcccgaatcaaacaaaaataatataacaaaaaaaaaatttcctaaacaactcaaaaaattcaaaacaaaaacaaaaatacaaacgctaaaactaaaacaaaaaaaaaaatattataacgtcaaaaaaacgaaccgcataactactaaactctcaaaattaaaattaaaaaaatataacctactctaaaaaaaaaaaaaaaaaaataaaaataaataatatataaaataaaaaaaaccacaaaaccctaaacccctaaattctaaaactaataccgaatataaaaaaatctaaatttcctcaaaactcctaactacaattaaaaaactaaaacccgaaactaaaaaaacaaaactctctcaaaactacacgaaactaccctcttaacctcactaatcaataactcccatttttaaaatcaaaaataaattaccctccatattccaaaacaattaaatcctcattaaccttaaaccctcttccctcactctcccactatcaaacaaaaataaaaaaataatccaaaatattaaccccaaaataactccaactatataaatctctaaactacccgtcaaaaaaataaaaaccaaatctataaacaactccaactccaacccaaaaaaaaaaaaaaaaaaacaccaaaaaaaaaccaaacccaaaccctaacacaactcaaatacataacaaacaatcttcccaacacccgataaacactaactactcctacccaataatccccactccaaccccaaaacaatcctcccaaacaccccaatacctaaaactaatctccgcacctctaatctaaccccatcaaaaacctcctccctctaaaaaatcaaaaaaaaaccctaaacatctaattataccactcctctcccaactctaaccctaaaaaataatatccaaacacaataaaaaaaataaaaattataataacaaaaaaataaaaaaatctaaaaaaaataaaaaatttcccatcatctcaaacttaaaaaaatacaaaaacaaatataaaaacaaatactaaaaaaacaaaaaaaataaaaaataaacaataaacctaaaataaaaatacacaaccaataaaataaacctaatctctaaaaaatcctacctataatctaaccttcaattctcttactacatcattaacctattccataacatacctaaatctccatactcttctaaatttaacccaacccttaaaaataatataactctaaatcttctctcctaaaaacacattaccctcgtacctaacttaactttactaaaaatccaaaatcaaccctatccttctctaaacctcaatctccatttctcaatatcacaacacatcctttttataaaaatataaatacttcgaaatctttaaaaaaatattctaaattctaaaacttctaataaccaaaaataaaaaaaaataacccctcccatttattctccccccccttaatccaccctccaacccaaaactttcacaaaaaataaaataaaaaaaaaaaaaaactaaataaaaaccaaactaacaaaaacaaaaactttatcccaactaatttctaaaccaaaaattccgataaaaaaacccgtaccaaacaaaaaaaaaaaactaaaaaaaaaaaaaaaaaaaacaaccaaaaaaaaaaaataaaaaatattaaaaaataaaaaaaaaaatttccttacaaataataataaaaaaaaaataaaaaaaaaaataaactcaaaaaaaactatacttacttacgttcgatctaaaaaaccctcctaaaaaaaaccaaataaatataatatccctaaaaacaaaaacaatctaaaaaacaaaaaaaactaaaaaaatactataaaaaaaaactctaaattcgaaaaaaaaatctaacactaaaaaaaacaaataaaattctaaatataaaaacacccaccattcatactataaactaaactcctactatctacctaatactatacatttatacaaaaattttaaaatactaaacactctcaaaacacataatcctatctcaactcataaaataataataataatacttttttataaataaaaaaataaaaactcaaaaaaaataaaaaacttaacttaaatcacacaataaattaactaaaactcgaattttaattctaaaaaaaaatcacttactaaaaaaataaaaatcaaaaacaaaaaatccataataatataaaaataaaaaacttctaaaaccaaaatatatataaacccacataaatataataaaaaacaaaaaaaaaatccatcctaaaatctaaccccatctttccttctcatatccctctatcaacctacaacataacccaactattcctacccctactaacaaccctaatcctaacccaaactcctacaactttaacaaatattctaaaaaaaaacaactcaaataaacaaccccacttaaaatcaccgtctctatcttattcaaccctaaccaactacttccaaacttaaaaaccccaaaaaaaaaaaaaattaaaaaactataaaaaaacgtccaaactaaaacacttaaaaataaaaactaaactataaacatctaataaattaaaaaaatcataaaacaaaactaaaaaaaaacactaaatacaaaactttaaacccctaaaataaaaaataaaaaatcctaaaactaaaaaataaaaaaaaaataaaactaaaaaaaaccctaaaaaaaaccctaaaaaaaacaaactaaaaaactaaactaaaaaactcaaaaaacgaaattaaaaaaccctaaaatataatctaaaaattttaaaaaaaaaataaaaaactctaaaaacaaaacaaaaaatcctaaaaactaaattaactaaaaaactttaaaaaataaatctaaaaaccgtcgaaataatcctaaaaaacctaaaaataaaactacaaaaccctaacccctaacccctaacccctaatcctaaaaaaaccgaaaaatcctaaaaccgaaaaaaaaaatcctaaaacgaaacttaaaaaccaccgaataaaactctaaaaatcgacaacgttaaattccaaccgactccacccgttcacaaaaaaccgcgcttttcgcgtacgcatcgcgaacgacgcgccactacaaaacgtactcgacgacgccctcaccatcccttaccacgtccactacctacgaccaccgccgaaccgccgaactatactaaactctccgcgaatcaaataaactttcctatcccgaaaccgaaaaacaaaaatactaataacgcgaaaaatacgcgtcaaaataaacgaaacctaccgattccgcgtaacactacctacgtacccaacgtcgctcaccgacgtctccctaacgctaaacgaactacgccccaacgactcaaatatctatcgctataaaatccaacacgacatcgataacaacaacgacgctataaaaatcaaaatcaaa

SEQ ID NO: 149 - DNA, 5414 bp, Artificial Sequence
  Synthetic
tacacaataaaacgcgatatctcccgaacctaaataaaatttcaatctaataattccttcctaacccaatcataatttatttaaaaatattattctacttctttcaaaacgctattcgcactataattaaataatactcaaacttttaactttaatttatttcatttcttaaaacttaaaacaaaactatacaatatcattttttttttatttccttaaaaattactctaactattattaaaataaaaattaaacacctaaacacttacttaaaccgtccgacacaaaccacattcattcacgtaaacactcccctttccctaccccatatccaaatttcgctaaactcacacccgacaacactactactaaaaattcccttcgactactatttattattttcctccacacaaaaaaaaaaaaaaaaaaacccgaaaaaatccaaaaaaaacaaaaaaaaattaaaaaccataaacaaaacccgtcgcgcgctcgttactaccgccttccccaacactctaacgactcctaaaaacaacgatcccatcttaaaaccgctattccgcccgactaaaatcaaaaataaacaaacgatcccctactctccaccgccgcttccgaaaactaaccacccgaaaattccccttttccactctccttcccactctatttttatcccaacgcgcgccaacgcctctcaaacctaccgcctactctcgcacctactcgccttccccaaacgcccaatacctacacctactcccgatcaacccccgtccgaattaaaccacccgcgaattcctacgtcgaaatcccgaaaccttctcaccctcgcctacaccctactccttccgctctctaaaaaaataacaacaacccccaacaccgcgaaaaatataaaaaaaataaaatccaaaaaaaaaaaaaataatatatatatatatatatatatatatatatatatataacaaaaaaaaaaaaataaataaaaaaaaattatctccttttacaactaaaaccaaaaatatatatccatctctaaaaaaaataatctacactaaaactaaaacaaaaataaaaataaaatatcaactaaaaataaactccgcaccgaaaaactataaaaataaaaataaataaaatttataccaacccccgaaaatatatatatatatatctatattataaaatatattcaacaacatatacgctatataatttctaaccttccctctccctatcaattaccccttcttcctttaattataactaataaaaaataataaatccaaaaacaaaatttaccaataaatccttccaaaactcaactcgaactatactaaatacaaaaaaaaaaaaaaaaaaaaaaaaaaaacaaaaaaaacgtatatatatacctatatatatatatatatatattataaaaaaaataaaaacaacgaaaaaaaaaaaaaatcgcatacgcacaaacgacccgaacctactccgcgactatccaatccgctaaaaactacgaaaaatcgaataaaaaaaaaccctacaacccctccgaccccatatctctttaacaccaaacacccgccgaaccgtaaaaaactcgtaaccgaacgccgacctccgctcgtattaaactaaaaattcaaaaccgcgcgcaaaacccgaattaaccgcaacgtctatattctcaacgataaccgaaaacctaaaatcaaaatcacctaaactaacgaaataaaaacgaaccgaataaaattaaaaacctaaaacttaataataaacaaaaaacgtaaaaaataacaaccaaataaaaaacactcttacctacccaacgctaacttaaaccgcaactttatttaaaaatttctttttccgataaaacaaaaacccgacaaaaaaaacgaaaaaaactaaaaactaatccttaaataaacactacccgacgactaaaacgcgaacctaaccatttaaataaaattaaataaaaacgcgattataaataacaaccgcgaaacgctacgaaaaaacgacgacaacaaaacacgaacgaaaacaaaaaaaaaacgacgaaaaacgcgataaaaaaacgcgaaacgaatactaaaaaaacaaaaaaaactcaaacctaaaaaaaataaaaaaaaaaaaaaaacaacgcgaaaaaccgaacaaaaaccgacgtttcctaacaaaaaaaaacgaaaacgcgcgaaaaaaaaaaaaaaaaaaaaaacgaaaaacgcgaaaataaacgcgaaaaaaaaaaaatataactcgccgaccgcgaaaaacgaaaacaatttcgaataccgaaatctacaactaacgacaaacgaaatcaaacatccgttcaaactaacaacaaaaacgacgaaaaaacgcgtaaccgaaatcaaacgtacaaaatccgaaaacgacgacgaataaactcaacttcgcacaacccttcccaactccaaccccgactaacccgacacttctcgaaaaatcccgacaaccgaaaccaataaatacctctacgaaccaacgccccgacgaacgaaaaaataataataatatccctaaacaacaaacaaacgtttaacataccgcacgacgacaacctacacgtaaaacccaaatactcgacactacacaacacctcgccgaactcctcgactcccatcgcgccctcgaccaactcccccaacaactcgaacaacgctaataataacgacgacgacgacgacgacgacgacgacgacgaaaaccgaaacaacaactccaacaacaataacaacaacgacgacgaaaactcgaaaactatacgaaaaacctatcttccaaccccaccgatacgtatttctacataatcaccgcttaaaaacacattttaacaaccccctttatctacttaatatttttttcatatctacacaacaaatcaccccacacctccaaccaattttcccctctctctctcttaaatattcaacaaatcttacctttcatattaatttttataacctaaaatattacctatacgcgtattatattatatttcgttatatctacaaactcactttcctcctcctcctacactctcgacttctttctataacttccctctttttctcttcacctctattttcaaaattattattattattattttaacgatctaaaaatattataaacgcgacgacgatatcgaaccctaaaccgaaacttccgaaaaaaaaacgtacaattccctactaaacgtaatatataccttctacttacaattacaaaacaatatattcgacgaactaaataaaaatctactaacccgcgccgaaactctaacaaccgtaaacatcgtctcccaaaacaaaaaccaccaccaccatccaccccaccacaaccccttcaaaccgaacgccacctaccacactataaataccatcccgtacacgtcgaccgcctcttcttcatcgatacccatctcgcacccttccgcgttaacgaacacgcaccaccaccaccaccatcaccaccaccaccaccaccaaccgcaccaaacgctaaaaaacgaactactaaaacacctaaatcccgaactaaccctaaacgctataacgaaccccgacgacgctataatatccacgccgactcacgcgccgcacataaccaccataaaccccatacaccaaacaacgctcaacataacccacgcgcacgaactaccgtcgcacataaactacataaacgacgtaaacgccgacccgcgaaacctaaaaacattcgccgaacgcttcaaacaacgacgcatcaaactaaaaataacccaaacaaatataaactccgcgctaaccaacctcaaaatccccgacgtaaactcgcttaaccaaaacaccatctacaaattcgaatccctcacactatcccacaataatataatcgcgctcaaacccatcctacaaacataactcgaaaaaaccgaaaaatcccaccgcgaaaaactcaccaaacctaaactcttcaataacgcgaaaaaaaaacgcaaacgcacgtccatcgctacgccaaaaaaacgctcgctcgaaacctactttaccattcaacctcgaccctcctctaaaaaaatcgccgccatcgcgaaaaaactaaacctaaaaaaaaacgtaatacgcgtctaattctacaaccaaaaacaaaaacaaaaaaaaataaaatattccgccgacatttaaaaaactcttaacctctccaaaaacgcccctttcctcgtccgctcttttctctcctctcttctacctcttttcacttttaacgactaaaaacaattccaataaatataaatctcgacaaatcgaaaactaaaaaaaaaacgaacgaacgaacaactaaacccaaaccgataaaaatataaaacaatttctcaaaaaaaaaaataacaaaaaataatatttatctattataacaaaattatccctttaaaccccacctcgacttcttcaaaaaaaatataaaaataactatttacaaaaaaacaaacgaaacaatatttaaaaaatccacaaaaataatcaaataaaatttatttttattcttacaaacatcacccgtattcaaatttaaaaatacactttacaactatttttcaaaaataaaaattaattcaaaactaaaactttaaactaaaattaatacttaatataataaaaacatctctaaaatattttaaattttaaaaaaaaataacaaattttctacatttacactatatattatatatatatttttattataattcttacccccttttccttctctaaaatattaatacttaaaaaaaaaattacgcctactatattcactaatcttaaaaactattattaaattattacaaaacaaccctctataaattattaatttatctctctaacaacttaattttatacacattctaattaattaaacttcttccgtctaaaaaaaataaaaaaaatatataactaataacgttcaaaaaattttatttaataaatttaccgaatttttacaactttcctcctatactatattccttttaacccatttatatattctcacttaaataaaaattatttttttctttatttttactaataatattctaatttataaatcgacactcaataataaatatcttaatcgtataaacctaattcactatctaaaatattatttacttcgttacatatttaataaaaattcccacattatccccataacacataaacgctctcacttacccttacacacacacacacacacacacacacctctaacaaaaaaaaaaaaacaattaaaaacataaccgatacaccattttctaattttaaatacatttaccacttaatatttacccttcaaattttaaatttcaccaaaatatttcaatcttccaattt

SEQ ID NO: 150 - DNA, 3381 bp, Artificial Sequence
  Synthetic
ttcatcatttctaaaaaattctcttaaaatccacaaaaaatctcacctcatctcgatcccctcaacaccgataatatcttaaaactcgaccccgattattcgctaaaaaaaacttaacttaaaaacgaaaaaatactccaaaattcctaaaaatccaccgcactccccatccgaaactcctcgcgaacgaaaccgcttctccaaaccgcccgcctccctaccgcaaaaacaaacccgcgcccgccgcccgaaaacctccgataaaaaaaaacactccaacgactcctccgacttaacaacgaccgaaccgaaaataaataaaaataataaaaaataaaaaaaaaaaaaaaaataaaaaaaaaaaaaatatataaaaaaaaaatccgaaaaacgaatccctataccgctaacgtcccgaacaatactaaaaaatataaactatattatcacgccgatatcaatctaataaaaattaacatcaaataaactatcattcatttccatatcaaaaacgcttttacaaacgacgacgacgacgcgacgacgactactactacgaacgactacctcaaaacgcgtatattttattccaatccccaaaccaaaatattattcattacgacaaaacaaaaaaaaaaaaaaaaaaaaaaaaaacaacaaaaaaaaaaaaaaaaaaaacaacaaaaaaaaaaaaaacaaaaaacaacgaaaaacgacgaaaacgtacaaaaaaaaactaaaaaaacgccgaaaaaacgcgaaacgaaacaacgcgaaaaacgacgaaaccgaacacgaaaactacgaaaaccccgcgaaccgcccgctcccctacccgaccaacgctcaaccccgccgccgccgccgccgccgcctcgccgcgcttaaacccgtaaccgaccgcgcattatcctcgaatacaaaaaaccgaactacgaactcgaatcgccgcgaacccaaccccgtaacgaacgaacgaaaaactatacgccaaaaacgccaaaaaacccgaaaataaaaacaaacacgccgacccgaacccgaatacaaaaaactcccgaccgaacgctccgcgaaaaaaaactaaaaaccgcgaacaaactccaaattctctttctcccgacttcgaacgtccttaaaaccgacgcttactccgcgccaccaacctaaaaatactttccaacgactaaaaacgaaaacgatcccaaactaaaaccaaataaccgaaaaaatcgaaaaaccgcgaccgcgacctaaaaaaacaaaacgaataaaaatttacaaacgaatcaataaaaacaaaaaaacaccgacgaaacccgaaaaaccaaaatccgaaaaactacacgcacgaaccctcacccaaaaaaaaacgaaaatatataaaatcaccgactttccataattactaaaaatctttaataaaacgaacgtaaaaacccgcgcgaaacttaaaaatcgaaaacccttctaactcgaaaactaaaaaataaattcgtaaaaaaaaaaactaaaaacgatcgaaaacgaaaaactaaaaaaaataacgcaaaatcccgaatctaccgctcaaaatttacatttctttcgaattaaaaaacgaactttcctaactcccgatccccaaaaaaaaacgaacgaaataaaccgtcctttcccgaaactcttcacgaaaaaaccgaaaatttccgcgcgataaaaaaactcgaattaaaaattaaaaaataaaaacttataaatcgacctaaattaaaactaaattttaaaaccaatctaaaattcgatttataaaatccaaactatttacgaaatcgaaatcgaaaaaccgataaataatttacacgccgaccaaaacaaaaaactaaaaatcgaaattaaaaactaaaaaaacgaataacgtttttaaaattcaataacaaaatcacaactttttcttataataaaaactattaaaatttaaaaaaaataacacgaaaaatcctacaactccgcgtataaaaaaacgtttaaataaacgataaaaataattaatctaaaccataacaaacgaaccccgaatttttactacttccccctaaaaatatttctttaaaaaaaaaaaacttaaaccacacaaaacccgatcctaaaaaaacgattccgaaaaacgaacaaatcgaaaaaaccttctaaacgaaacgacaaaacaacctcgcgaaactaaaaataaatctaaaatcccgacccaaacgactcgaaatactccaaaaaccacctaaatctacgaacgcaacgcgacgaaacgaaaacgataacccgcaaaaaccgcgacctacgataaaaaccgaaaaacaacgctaacaacgaaataccacaataaaccgaaaaatctaaactataacccaaaataaaaccgactcaaactccaataccctaattaaaaccgcttcctatacttacccgcgccgaactaaaaaacccacaaacccgacctttaatacgcccgaaaaaaaaaaaaacttaaaataccctactatcatctctcctatccgaaaacttcaaaatattaacttttcgaatttcaaatactctataaccgataacaaccaaaaacttcgcattttaaacaatttaaacgatcctccccgccccaaatctaaaaataataatattcaaacataacacgatatattacccaaaacccctacccttaccctcctactctctctaccgttctaaccttcgaaaaccccaaaaccttaataaaaacgaaaaaaaaaaatcccgtttctaatctcataatctcccactcgaactacatccccataacttaaacctaactataaaatataaaataaaaaaaaatcccgaccaaaaaaataaattattctctaaacatctaaaaaaaaaatatatataaaaaataaaaacgaaaaaaaaaaaaacattaaaaaaaaaaaaatttaaaatttcctcctcttaatattaaacccaccatcccaaccccctacacccctaacaaaaaaaaccaaacaacccttacaaaacacgaaaaacgcacgacccacgacccacgaataacctcgacgacccgaaacgcgacgactaccgccgcgaaactccaaaactattattttataaacttaaaattcgtaaaaactctctcccgctaaaaacccgaaaaaaaaaaaaaccgtaaccccacgaaaactccctaatcaccgcctcccctttataaacgaaaaaaacttaaaaaccaaaaaaaaaaaaaactactcaaaataaaaaacaaaaaattaccgaaaaaaaataaacccgaccaccgctcacaataaaatacctcgtacgaattacactaaaatatacccaacttaaaactaactattttataatcctctctaa

SEQ ID NO: 151 - DNA, 1266 bp, Artificial Sequence
  Synthetic
ccttccattaaacctaatcgcgaaaaaccacaaaaaacaatcaaataattttcaaaaaaaaaacctctaatccactttccctccgccgccaaatctacaaattcctaaaaaaaactcttctcaaatcctaaaaattactaaactcttaacgaacgactcatctcttaaaaacctaaaataataaattacaaacattaaaaaacttttaataactttaaaaaataactctaatataaactcacctttccaaactaaaaaaccaaacaaaaaaaccccctttattatcttctaaaaaaaaatcaataaaaaaaacgaaatttaaaatataaattaaaaaatcctacgtctttacctaccacctctacctaaatcataaaaaaatcgaaaaatacaataaacttcaaaaatcatacaccgtttccctaaaacctatccaaaaacctaacttctaaacccaaaaacttctacaaaaacaaacccactaaaccaaacaatctacaccaacacctctacttctaaaattctatttcgtcttcttctattaaaaaattaacctcttaaataatttatatactttccgacaaataataaaaacgcgtaaaccgacgaaacgactaaccttaccatactcgctacgcaccgcgcccctaaacgttccgaaaaccctacccgaactcccgcgaaaaaaccccctcaaaatcgccctacgtctaaacgccgcgtactaaaaataaacgcgcaacgactacgcacgaaaataacaatacttacccgtaccgctaaacaacgccttcgaacccgccttcctccgcaaacgcaacgaacgcgaacgacaacgaatacgctacgtaaacgaaaactatacgcgcctccgaaaccacctaccccgaaaactaacaaacaaacgcctcaacaaaataaaaacgctccgcgctaccatcgactacatcaaacacctacaaaaactactaaaacgccaaacctaaaaactcgaaaacgcgaccgacgccgtcccccaacgcaaaacgaaatacaacaacgacgaaaaatccaaaacctcttcgacgccttcgcccaacaacgaacccgaaaaaaaaaacaactaacgaacgcccgaactaaccaaaacccccgcgcccgccgcacaacgcgcaaccgaacgctcaacctaaaatcctcttcgaaaataatttacattcttaatctaacatcttctccaaacctaaatcttaaaaaaaaaaaataaatactaaaatttaaaaaata

SEQ ID NO: 152 - DNA, 2968 bp, Artificial Sequence
  Synthetic
ttacaaccccgccctcccaaacttacaaaaataaacgaaaaattatattaaaatttaaatcgaaaactctcaaaacatctcaaaaatactttctctattttttttttcctataaatattaaaaaaattaacaaacgaatcttcctaaaaacaaaaaaatatataatttaatattttctttaaatttctatataaataaatacacaatactccgtattatataataaaacgaaatatatcttcttattaataaaatacactacgcaaatcaactcgataaattaaaataaaaaaaaccgactacgaaaataaaaaaaatataatattaaaataccgacgcttataaaaaaaaacgcgaatataaacgtataaaaacgaaaaacgtaccaaaaaaacgcgaaaaaacttactaataaaacaaatatacgtctatttccataacgccctaactcgcgacaacccctaactaaacgaaaaatataatataaaaataaaataaaaaaaaacaacaaacgaaacctaccacgtcacttaaaaaatatatattaaaaaaaaaaaacaaaacgaaaaaccgaaccgctacaactacgacgacgacaacgaaaccttaaaccgtaaaaaaataaatccccgcgctcgaacgccgaaacaaccccgaaccctctacgaaacctacgacgcgactcctaaaaaaaaaataacgactataacgaccgaaaccgcgaccttaaccgaacccaaccccgcgataaacgcaaaacgaaaaccgaaccccgccaaaaatctttaccgaaccgaaaaaaaacgcatctaacgcttcgataccaacgacaaccgaaaatccgaaacgactaaaaaaacgcaataaaaactaaaaaaaactaacccgactaaaaaacgaacctctacgtccgaaaaccgaatctcaaacaccgctaaaaacgaaaccacgcgtcttttcgaacaaccaatttcacacgcgcctatatacgattccgaacatcccaataaactctaacacccgaacgcgaataacgaaaaacgcaaaaccaaatccccaacgcccaaatcacctccccaaacccaaccttacaaaaaccaaaactttaaaactcacgaacccaacgaccaaatcaaaccgcgaaccgaaaaaaacgcgaaccccaccctaaaaaaaacgcaaccgaaaactaaaaaacgaataccgcgctccaaaaattatatcgtaaacgccgtcctaataacgaaaaacgcacctccgaaaaaacataaaatcgaaaaaatcccttacgctaacgacgccgaaaaaaatccgtaaaccgaaaaaaaaacaacaaaatacgaaaaacttcccgaaaaccgacgaaaacgaaaactactataataacgaacgactaataatcaatatctccgaactacgctttaaaacacaattacgcaccctatcgctatttccgaacacgctactcgaaaaccctaaccgacgaatccgcttcttcgaccccctaaaaaacgaatacttcttcgaccgcaaccgacccaacttcgacgccatcctctactactaccaatctaaaaaccgcctacgaaaaccgatcaacgtacccctaaacattttcctaaaaaaaatccgcttctaccaactaaaaaacgaaaccctaacgaccttccgaaaaaacgaaaactacctacccgaaaataacgaaaacgaaaaaccgctaccctcccaacccttccaacgccaaatataactactctttaaatacccaaaaaactctaaaccgaccaaaaacatcgccatcgtctccgtattaatcattctcatctccataatcatcttttacctaaaaaccttaccccaattccgtataaataatcgaaataaaaacaataataatataaatcgaatctccccaatttccaaaaaaaatcaaaaaaaaaaaaaaaataaaaacgattcctacacatttcatcataacatcacccctaaaaaaataaaaaccgaaaactcctcctcactcaatactcttaaaaactccttctttacaaaccccttctttctaataaaaacgctatacattatctaattcacttttaaactcctaatacgcttctccgcctaccctaacaaaccgaccttcttccgaaacatcataaacatcattaacttaataactatcttcccctacttcatcaccctaaacactaaactaatacaacaacaaaaacaacaaccaaccaataaaaaaaacgaccaaaataaacaacaaaccatatccctaaccatcctccgaatcatccgcctaatccgaatattccgcatcttcaaactctcccgccactccaaaaaactacaaatcctaaacaaaaccttacaaacctccataaaaaaactaaaactactcatcttcttcctcttcatcgaaatcatcctcttctccaataccgtctacttcgcaaaaactaacgataacgattcgctttttcccaacatcccgaataccttctaataaacaataattacaataaccacgataaattacgaaaacatataccccataactataaaaaaaaaaatcgtaaactcgctatataccatcgctaaaatcctcaccattaccctacctatacccgtcatcgtctccaacttcaactacttctaccaccgaaaaacgaaacaaaaaaaacaaaaccaatatacccacgtcacttataaacaacctacgccgaacctaaaaacaactaacaacgaacttaacaaacctaacttccccgaaactaaccgaaaacgaaaacccaactaccttcctacaccacatcgaacctatacaaaaaaaaaaatactcacgaaaatctaacccatacaaacaaaacctacaaaaaaaaaacactaaactaacaatctcttaaacttccttctcatttccactactcactctaacttcaattaacttcttaactctctcccc

SEQ ID NO: 153 - DNA, 4899 bp, Artificial Sequence
  Synthetic
aaaacaaccctaaaaaacactaatctaaaaaactaaacccttaataaaaacctctaaaaaaacgcctatcttccgaaaccaaacaaaaaaacgtacaacgaaataaaataaaaaatactcccaactacaaacacctctactcctaactatctacaaaaaaacacacacacacacacaccctaaatatcaaaaaaatcctcccttccctatacctcctccttcccaaacccctaaaatccaaaaatacctatttccaaacgccaaccctactaaataaatataaaaataaaaatcactaaaaacccaaaactaatttattatttttatttcctattaaaaatactataaaaactttacaaaactaattttaaaaaatcttaaatttaacttcatctaactcctctactaaaacgaaacgtacaaccaaacctacttaaacctccccaaaccacgaacccgcgacaacccaactcaacacctacaaacgaaaaaaccgcgcgcttaactcgaaaaccccgccctaaacaaaaaacaaaaaatataaaatcaaataaaaaattataaataactatcctcccctcccctctaccgtatttcctaaatccgtacgctatacaaaaaaaaaacgaaaacgaaaacgcgaaaaaaaaaaaaaaaccgtttaccattaatctaaaaacaaacgactaacctcgataactaatattttcgcttttcttttctcacttttctcaatataaaaaaaacaatcaaacccgcgcgaaacgattataaaaaactctactaaaatttaacaacgcgaaaacttaaaaaaaaaccccatactaactcccattcaaccgacccgttttcctcgaactttaaaaatttcactcaaccgtacactcaataacttcacaaaactaattacaaacttcaacgcattcctaaaaaaaccaaaaacgacgcaaatacaaacgaaccgaaaaaaccccttatcccgataacaaaataaaacaaactaaaaaaaacttaaaccacacaaatccaaaactcaccaaaccgcaaaaaacctaccttaaaaaccgaaaatcattatccgccctattcaacgaaacccgaaaaccctaaaaaccgaacgaaaccaacccgaacgaaaacaccgccgccaacgcgcgccgacgacaaaaaaaaaccccgctaaacgctacgaccaaaccgaaactcaaaacgctaaactatacgtttacacaaatctatattccgacgaaccgatatcaaccctaataaaaacacgaaaaaaaaaacacgccaataaaccacgcgacgcgtaaacaatacgcaacttctatcgacgtccaaactatacgaccacgtttcaaccgatacccccaaaccataaacacccaactccaaaatcgctctacgccctttctccccaacccacctttcccattaatcccctctcctatccgaataacaacgcgaccccgcaaaaaacgaccccgaaaaaaacaataaccgcacgcgtaaaataaaaacgacgaaacacaaaacgataatctaaaaatattcgcataaccgaaaacgctttcgctacgaatttaaaatcgaaaaacaattccctcccgaaatcattaacttcgtctcctcgtactcaaaaaccaacaacttaatactacgaacgccaaaaaaaaaaaaataacgcctcgcgaaacgcgcgaaaaaaaaaaaactacgacaacgaaacgcgaaaacgaaaaaaacgcaacaacgaaaaaccgaaacgcaacgaaaaaaaacgctaaaaaacgaaaaacgctcgaacaaccaccctatccccgtctaaaaccccgcgctacgtctaaaaaaacgcaacacgcaatccccgcgaaaaccaaaactcgaaaccccctaatacataccccctctcctcgcgccctaccgaaacctcgaccctacctaccgtaccctaaacctaattatacccgaaaatccccgcgaacaaaacgcgaaacaaacaaaacgcgcgcgccgacgctcttttatctaataactaatttaaattaatacgatctttatataaaactaacaacgaataattatctattttctcgccaacaatctccatcacaatcacttatctaaaaacctacgattaaattaatcgttatattcccgaaataaacgtcgctacaatccgcaacgaacgcgaataaaaacctaaaaacgcctcccgaacgctaacgaaaccgatataaaccaactccgctaccgcctaaaacgcgatatacccaaatattaatacccgcgaaaaaaaactataaaacgccttccccaaaatttacttaacttacttttcgctcgaataaataatacgcgaatacgaaaaacgaaaaaaaaaaaaaaaataaaaaaaaaaaaacctataaaactacgaacccgaaacaacctaaataaaacgcgacctcgaaaaaccctaaatacatcgcgacgaaacctaaaaaaacccaaacgccgaaaaaaccgtcgataccgaaatacaacgtatttcacttaaaaaaaacgttaccctcgatcccttacctccctcctattatctcgatttttctaacttcgtccttcgtcccacccacccgttcccacctcaaatccctcccccaaaatcccctccttaaaaatccaaatctctaaaaattattaacaacaattacacgccccacgattaaaatccacaaaaaaaaaatcccaattctacaaaccgctccaaaactaaaaataaaaataataacaaataatacgtcaactctctaaaaaaaaaaaacttacattacaaaaacttatctttctaaactaaaatcaaaacgaaaacgtcaaacgcgctccgtttaacgacgataaaaaaaccgcgcgcccgcgctatcccaaccgaaactaccctaactaataattaaaaatttaacgtccgaaattcaaacgcttctacaactcaaatttaccgaccaaaaaacctcaattacaacttttcaaaataatatttctaaaaaataacaaattcaaactcaactaataacaacttttaactataaaaaataaaactacttccctttaacgataaaactcttaaacttcgaaaaataaaaaaatacaataaaataaaataccataaaatcactaaatttttcaaaaaaaaaaaaaccccaaattactcccaaaataaaactttataaattcttattaaaaatctttaaatctcgaatttccccctacaacatctaataaatactttaaaaacaaacgaacaaatcccacctcgaaaatcaacaaacccaaactctaaccaaaactctccccgcgttttcttctcgtaacctaaaaaatatcccgccccatcgctcacctaactcttatcatctcgctcatcttaaaataacccgtaaacaatactactccctaccgcaactccgccgatcactcaaacaaaacgaaatatacaaataacctaaaaaaaccgaaaccgaacactacgcccattatcccgaacgctcaaaacgccgaacaactacgcaaactttctaaactcgaaccctcgaccgccacgctaatatcatcattacaaaacatcaaattttaaaaactatacaatctaaattcattaacaaccttctcgacgaaaattcttttccccgtaacaaatttcaaaatccaaaccccctccaacgttaataaaaataaaaaaaaaaaaaaaaacccaaaaaaaaaaatctttcgcaaataaaacctcgaaaaatccctactctccttctaatacattccgcaactttaaaaaccttccttaaaattctaaacgcgaatatctaacttaaataacctaaaatttaaacatattcccgttttctcccaaaaattactttaatattacgaatttaaattcactaaaaaaaacccaaataccaaatcgaataaatttcataaacatcatcttctattttaataatcgcgacaataatcgtttctaaaatcataccctttaaaataaactcacaacaatacatatcaaaaaaactacgcgccctaaaacaaaacaattaaaacatcaaaccacgctcctcaaactacaaaattacaaacaactcacaaacttccaatccccaccccaaaataaaaaaaatctcaataacttaaaaacctacttacactcccgctttccacgcccgatcccctacccgcacctacccaaataatctcgttcctctaaaatctaaataataaaaaaaaatactcctaataccccgaatacaaaacgaacaccaacgaacgaaacccaaaaacgctaaaacccaaaaacgcaaaccaccgttaaaacctaaaaaacgcaaaaacgaataaaataatatttctcaaattcgtaaaaaaccgcaaacgccaacaaaaacaattttcctcccctaaacaatactaattctcctcaaacccaaaatctccactaacgtatccttccacctcctcacaaaactccaaactactatcccctctctaccgcatctaaaactaaaaaaacttcctaaaaaaaaacaaaaaaaaaccaccaaaccaaaaccgaaaactaaactttaaaatcgaaaacgacaaaaaaacaaattctcccctacacaccccaaaacctctcctacacccgcgaaaccttccttaaacgcccaaacccccgaaatacctaccctccttctaacaaaaaaaaaaaataaaatataaaaaaataatacaaccaacaatatttttataaacacaacaacaaaaaaatacataaaaaaaatctatttattaaatatatcatttttaataaaaaacgaaattaaaaataacgccatctaaacccaaaaaaaaactacgaaataaacgaaataaacgaaaaactcttaa

SEQ ID NO: 154 - DNA, 2346 bp, Artificial Sequence
  Synthetic
actcaccttataaacactcgaacataataaattaacattaacattactattaaaaaacaactcaaccgtattcttaaccataaccatttaaacctattctaatacaaaattctaatccaaaataccaatataatccctcaaaaaaatactaaaaatcgtcacttatacctattctaaacataatcaccgaaaactatcctataaacattcacttaaaaatcattcgaaataaaattactcctaaatacgttctccttatactctatttcctcctcctaatatctctatataaaaaaaccctcctcactcaaccctcgacgaccctctaataccctaaacaactccccgaaaaacaatctaccgctaaacgacgactactaaaaaaaaaaccctcctaacgcgaaatctaccgctccgaaactcgctctccgacaaacccgaaaaaaaataaaataacaataaattaaaatacgcgcgtacctcataaatacgaaacaaaacgaaccgccgaaatataaatcaacgcgctaaaaactaaaaaactaaataaataatcacgaaatctcactcacgctcgactcctccacccatcccgtctacaacgcgtatcccaatccaaaacgtacgtacgctcgatatccgattccgaactatatatatccatttaacgaaatatcgaaaacgaaaaaaatatccttatcgatatatctaaacccaaattaaaaaacttctcctccccacccccgcgtaaatataaaaatatatccgaactaaaacgcgtatacttctatacctatacgtacgtatacgaatcaaaataataaaaccgcgcatcaaaacaaaatacctacgtctacgtctaaatctatctaatctacatatcgacgcgatctcgacctaaattcgtatccctaaatatcgaaaaaccaacgtaataaaaatatccaacctcccgaaaaaatactataccttaacaccttcgtttcaccgccccaaaactaacctaaaactccgtaaaaaataacctacataaaaaaaacccgcgtactatatttctaaaaaaaataaaaaaataaaaacgcaaaaaacgaaccaaatccctaaacgcgacgcgaactcgaaaaacccgcgcgactaacgtcaaaccactccttaaataaaaccgacaacgcgctccgctcgacatttcccgaaaaaccaaatcgcgaccgacgccaacgccaccgtccgatccacccgccaacccgcacaaccgcgccgccgccgaacgtttcgtaaacgacgctccgaaaatcaaaaataaaacttcgaacgctaaacgcgctccgaacccgacgcacgtaaaaacctaaaaacgcccgaaccgcccgactacccgaaaccccatcgcctaaaaccgaaaaatactaaaaatacaaccgcctattccccgaaaaaccgctacccccgaaaccccctaacactatacgcaccctaatcaacaacccccgaaaaaaacgacgcccccaacgcccgacccgcgtaaccgtaacaacgccacgcgaaccctctaaacgaccgcaaaaccacaacaactcaaccgccgataccccctcgaaaaccataacccccgacgcgaacccataaaaccataacctataaaatcctaaaccgcgcgaaaccacctcaaccgcgaaaaacgcgcaaactactcttcgccttcacgctctcgctctcctacacttacctatattacaacttcctatactactacgacgacctaaatcgaaaccgcctcctcgacgcgcctcgctacctccgcgaccccaacgcgaacgaccaaaaacttctccaaaaatcccgcccctataatccctccgaaccgacgcccaacgaacccaacgctcccaacgcgcccgccgccgccgtacccgcccctcgcctctccgattccaaccactccgactcacccaaactaaataccaaacgattaccccaaaccctcattataaacgtaaaaaaaaaaaacacccgaaccgtactaaaatttatccgaatacacccgaacgtacgaaccttaaacacgaaaccccacttctttaacaaaaactacgaccgcgaactaaattaatacaaataaaaaccaaaaactccgctccgtacgccgaatctctaatcgcttccattaaaaaaaccatccgtctcttatattttctctttcttttaacccaactcattatataaattcaaactaacacacaaaaccataaaaaactataacaaaatttacccaaaacttcccaataataatctaaacgaacaatttctaaaactacaaaaaacgttccctcgtcactaaaatcgtta

SEQ ID NO: 155 - DNA, 2695 bp, Artificial Sequence
  Synthetic
attcccttccacgaaaatcccgaaatactttaaaaaaaaaaaaaaaaaacgtcccctctaaaaaacccactctccgaacttctactaacctaatctccgcctcaccgacctcttacgaccgctacaaaaacgcactttactaaacaccccgaaaacgtacctctcgcacaaaaaacgcccgtctttactaaaactaaaacgacgcttaaaaaccgacactcgatcgctattaaactccctcgcctaccgcttctaccgaatcaaaaaactaactatcgccgcaaccataactactcaacgaaaacctcaaaccccaacctctactacgccctccgacttacgctccgccgaaacgaaaacaaaacctaaacgaccaaaaaaaaaacaatcgcgaaaaaacaatactaaaatttaaaaaaactacaatatcgaaaacccgacgctcacaaaattaatcaaaatctaaacaataacgacaaaatatataaaaatccaaatataaacttccccaacctctaacgaccgaaaaacgaaacgaaacgatcccaaaccctcttacgaaataaacgtttacaccccaaacttacaccccaaaacgatcgacgtccaaaaaacaataaaaaatttaatcacactacgttcgaaataccaaataaaaaaaaaaaaacgatacccaaaataacaaaacgtacctctattaaaaaaacgcaaacgttataaaatatccaaaatatacctacacatacatacataaaaaacccgtttacaaaacaaaatctaaacccaaacgaataacgcgcccccgataaaaaatactaaaaaataaataaaacgttcctttaaaaaacaaaccaccaaccgcacgaaaaaaaaaaaaaaaaacaacaatttaactccctacgacccgcgattctaaaaattaaaaaatccgtcccaacaaaataaaatctacaaaatacatcgcgccgactacgactttccaaaaaccgaccacccgaattctaaaattccgaaaaacgcgaaataaaaacgattacccgaaatctaaataaaaacgcgaaacgaaaacaactatttccaactacgataaaaacaactcccgaccaacaacactacaaaaaaaacgaaaaacgaaaaaaaaaaaaaaacgcgaaaaaaaaaaaaaaatcctcgaaaaccaaacacccctcgaaaaaaaaccaacgaaaaacgatctacgaaatcccaaaaataaacgctctttctctttccgcttactttccgacacgaaacgaacacaattaataattatttaaaaaatcctaaatctaaaataactcaataatttaaataaaccccctcaaaaaacaacgataccgaaaatatcctctccaactcgacgcccacacgcctttaactaaaactccccgccataatccacccgaaaccgccgcaccgaactaatctccgcacaaactcaaaaaaaacgaaaaaaaaaaaaaaaaaaaaaaacgccctaacgaactcgaaatcaaatcatcgccgcgctactacccgtaccccctaaactcgcgcgccccgacaatcaacaactcacaaacaacaaatcaaataaaaattacccgccgaacgcaaaaccgatcactcaatcccgcgccgcccatcccgaccgaaaaaaaaaataacccgcgcgctacgaatacccgcgcgtccgctcgaataaaacgaaaactaactacaaacgatattaactcgcgacgactaaaactcctaaccgaaactacccaccataatctaacgccaaaaacgcaaacgaaacccctaaacctcctaaaactacctcgcgaaacaaccgaaaacgcaacccgaacgcttaaaaccgaaaacgaaatcaaaaaccgaaaccaaaaaacaaatacaaacgactaccaactcgcccaacttactacgcgaataaccgctcaaaaccgcgaacttacgaaacgccccccgccgccgcgccgccgcctccccaaacccgaaaaaaaacgctcaaaataaaatcccattcataaactaaaactctaaacgcgcgaaaccgccgccgcccctccgactaactcaactaaaatactaactccgcaaaaaactcgaaacccgaacgaaaaccaccgaaataacaaataaaacgcaaaacccgcgacaaccaaaattcctcccgcacgacccgccgacccacgaaaaaacgaaaaaacgcccaaataaaaccgaactaaaaaccgaacccctaaaacgctaaaaaacacaacgcgctctaatcaaattccctttcctaaaaccctccgcttccaaactcccttctttcctccctccctcccgccacccctctccctcctctctatatcttctatctctccccttttctcctctctacgcaatcctacgtaattaaaatttaaataaaaaattctcaaaaacaaaacgaaaaaactacaacttaaatctactccgtccgatccctcccacaaaaaaaacacaaccacaataaaaattaaaaaaccctaaatacgcaaaaaaaaaataaaataaaaaaactaaaaaaatacaatccacactctctccaataaacttaaacacgtaaaattctctatttaattaaaaaaaaaataaacactaaaaaaaataaaaataacctcttaaaccttatcgtaaaccccacctataactcattttaaaa

SEQ ID NO: 156 - DNA, 2837 bp, Artificial Sequence
  Synthetic
aataatattcctactaccatcccaactaacttttcgtttatccaaatcgctctctaatcgttatcttttcacttcataaacgccactaaaaattatacccaccacaaccccaccccactcccaatcccactcccaatccccaaattcaaattataatatttaatataattcttaaactccacataacccgctccgaaaacgcactacaaaacgcaccccaaatactttcccaaatctattccttcattctctcatttcaaaaaatcgctctatttattcaccctaaaaaaataaactcaacccccgctttcgcgctaaaaaaaaaaacacaatcattctctctttctcttcccctccctccccaacaccttccctccctcctcccttacccctcctaatctccaaccctaaaaacaacactcaaacatacaaactttctaattccttccaccctcccctatcccaaacactaaacaattaaactaaaaataacaaacgcaaaaaaatctaaatttccatctatcaaaaaatcccctttccccacacccgctctcatacccccgccccctcgaaaaactctaaaaacacctttatcgcttctcccctaccctctaaaaaaccacccgtaactaaaaactcccgcgcgacaactacaccaacaacaaataatccaaataatacgaaacgataccttattataacatcctaccctctacaaaaacaccactaaaaaccacataaaataacccccaacaatcctacaaaaacaaaaaaataaaaaaaaatctatacaccctaccctataccccaatctctcccaccctatcctccgcaaccgaaaaaaccctaacatctaaaaacgaactaaaccgaaaaaacaaaatcaataaaattcaacaaataaacaactaaaaactaaatactactaaattctaaaaaaaaaaaataaaaacgaaaatatccgaatcgataaaatataaatcgtatctactacataaaaaaatcgtacaaaaaaaaccgctctctataaaaaattaaccaacgcctaccccgaacacccccgcgaaacctccaacgaaactcaaactaacgacgactcaacaaaaaacgaaaaatactaaaaaacgccgaaactacgaacttacgaaaaaaaatatactcgatataactactccaaatcttatcgtcgcgatactaattaacgaaaacgacgatacccgaaaaattacacgccaccatataaaaaccgacctcgaacaaacgatcaaaaacctactccaaataaataaacttaaaataaaaacgaaacgtatacttctccgactaccgatcgaaatcgcgactctcgttaaacgtatccccgaaaacctccttaaccaacgcgatacgcccgcacaccataatacgcgccacacgccgaaatttaacgtcgacctaattatcgcgcacgataatataaaaaccccgataacccaacgtaaaaaaacccgaacgcttatcctacgcgccgccgccgccgccaccaccgtacgctcccgaacccgaaaacacgacgaccgccgccccgcgcaacaacaacgcgtcgctactaccctacgaaacgttatcctccaaatcgctctaacaaccctcgtcgttaaaaaaattctacttaataaccttaaacgacaacaacttaaccaaatcgataaactaaaaatactcgacctcgcgcaacaaccgctccttctcgaaaaaatactccgacaacgcgaattacttatcccgcaaataatccaacacgtacctaaaaaaaaaaccgtcccgatcgataaaaaaacgcgcccgactatccctaaacaactcgccccgacgccgaacgccgccacgaaaactaaaaaacgaaaacatactaaccaaaatactatccgaaacgctaaacaacgtcgaatacttaatcacataaacctaaccgcctacgttcaactccactacttcaaaaaaaaacgaaaatacgcaaaaccccgaaacgacgacgaccgacgcgccgaacgaactaaacgaaaaaaccatctcgctaataaacaaaataatactaccgccgctacccgtatccttcaaaaccataatccccccgccgccgacccaataacccgaaaaaactacactttctcgttcccgaaacccgcgccccaaccctccgcgtactcctaacgctctacgccctcgaactaaacgacgcgttcctccgaccgaaacgaccccgctcaaaattcgaaacaacgacgacgtcgacgacgcccgaactccatcgaaaaaaaaacgcgcgaaaaaaaaactccgccgatacgacgacgacaataaaaaaacaaaaaaaaactactactcctttaaaacgaaaacgaaaaaatataaaaaaaacttacgaaaaactctaaaactactactcgactctccgtaaaaaaaacgaaaaaaaaaaccgaaacaaaaaaaactcgccgccccgacaacttacaaacgtaccgcgaaaccgaaacgccgacgataacgctaactactcctaacaacacgaaaaaaaacactaacgtcaaaccccgaactaaaacccgaacctcaactatcatttaaaaaaataaactacaaccgcgactcccgaactccgacgctaaaaaacgaaaccgaaaaccgaacctacaaaaaatacaactccccgacccgcgaaaacaaacaaacaaaaaactcctaaaacaaaaacgaaaaaaaaaaacaacgctctcattccttcatttcactccctccttaaatctataccaccccctttcctccctcctttccttcttctcttccttccttcctta

SEQ ID NO: 157 - DNA, 7618 bp, Artificial Sequence
  Synthetic
aaaaatccaaaaaattctctcacctaacacttaacaaaatcaaaaattcaccctccaaaacactctacaacccaaaaatataaccaaaacccttaaaaactcccaatctatacttaaatataaaccgaaaataaaatataacccaaaaatcgcaaaaaaaatttcaaacaactaaaaaaatacacgtaaaaacaaaaaaaacctaaaaaatataaaaaactaaaaataaaaaccaatctacctactaacctacaaaaacctctaaacttactctctaaaaacaaaatccaacgaaaactaaaaaaaaaaaattcctatacgaaaccaacctaccctccccctccccactcaatataataaaatccctaaaaatctacgccctccaacgaacaataacatactaaaaaaaaacttaaaaacaaataaacaacaaaaccacaaaaaaccaaaaaaaaaaaaaccactaatataaaacaaacgtatactcataatacacgcccaactatacaaattattcataaaaaaccgctataaacgctaaaccgtctcaactcaatccaataaaaaaaaaaaaaaaaaaaccctaaatacttttaaataacatataaacaaataaacttcccaccttccaaacaaaacataaacaattaataaattccttttaaaaaaaaaaaaaccgaaaaaataaattcaaacaatcgtaacaaaaacgtcctatttaaaaatataaaaacaaataaaaacaaaaaaaactaacgaaattaacaactatatcctccgataaaaataaaataaaaaatctcttctaataataatttatactctttataaaaataaaaaacattttaaattatatactaaataactctaatacaaataaaaactaaaaaaaaaaaaaaaaaccgaacaaaaaaaaaaaaaaaaaaaaaaaacactaaatctctctaaatttaaaccgtttaaacgtctatataaacatttttctttcttaaaaaaataaaaatacgaataataactacacgaatatacacatttatcaaaattcctctaattacactcttaaatttatacttttctccctaaatacattcacttcccattttaaaaaaaaaaaaaaaaaaaactttccatcttctacctaattatacgttcgaatttcgtaccttaacgatacattacttatttaaaaactacatacatcataaaaaaaaatctttatcaaaaactctaatcgaacgcaataactcaaacctataatcccaacacttaaaaaaaccgaaacaaacgaaccacttaaacttaaaaattcacgacgaacctaaacaacatacaacaccccttctctacaaaaaataaaaattaaccgaacgtaataaccgcgcctaaaatcccaactactcgaaaactaaaataaaaaaatcactcgaacccaaaaaataaaaactacaataaaccctaatcgcgacactacactccaacctaaacgacacaacgaaccatctcaataacaaaccgaaactctaactatctccaaataatcgaacttaaaataccttttattttcaatacttataattttctaaatttttaaaataacgaacaaaatctccttacgacattaaatacaatacaaccgccgaaaaaacgtcgaaaacaaataaaattaaacgccgtccccacttcctcccctacaaccgcctccaaaccgccaacctcgcccaaccgaaacctcaatcctaaaaaatcgaacgacgacgaccttcccctaaaataacattcttattacccgtaaaaccccctccccaaataaaaaacaaaaaacgaaatctaaaacaacctccgctttctccttcctccttcccccgcgacttccctactacgccgaacctcccaaatctcaccccgaatttccgcacccgataaaacgcgaattccgacccaacttctctcgaaaataacgactaacctaaaacccatccctaacctcgacctccgcacccccccgcccccctacctataaaacccgcaacccaaaaacctaaaacctctctacgaacacgcaaaaatctcccaacttccgaatcgttccgccgttcgccctaaatctaaatctaaaaattcgaaaaacaattttaaacgtttctaattaaattatatctaaatcgtacattttacgcgacaaaaaactcgaaatccttaaaaatcccaaactaccgcaacccctccctcctcctccccttacccttacccctacccaaaaaaatacgctcctccaaaatcatccacacgcgccgtaccgaaccccgaaccccaattccaaaaaccgcgtctctacgaaccaataaatcctaaaaaaataaaacttcgaacgctcgaaaaaaaaaaacgaaaaaaaaataaaaccgaaaaataacttttaacgaaaaaaaaattaaaacatcaactaaaccattaaaactaatacgatatactataaaacgaaaatcaataccgaacctcgaaaaccgacgtaaaaaaaaaaacaacaaaccgaccgcccccaaaccgatcgaaacccccgtccatttaaaaaaaataaattttcgcgatttaaaaaacaaacccaaatcaaataaacgaaacccgaatatactaacgctacgattacgcgcgcgaaactaaaaccccgaaaacgctctaaaaaaaacgcaacgacgaccgcgaaaaaaaataaaaaaccgaaacaaaaaaatccgaaaaaacaaaaaaaaataccgaccgacaaaaaaaaaacgaaacaaacacaccgaaaaataaaaaaacaaaaaaaaacaacctcgcaattttcgcaaaaaaaaacaaaccgacgcgactaaaaatacgcttctaaaccaaacgcgacaaaaccccaaaaactaaccaaaaccccaaaacccaaacgcctatttaaactcctttaaatccgacaaaaaacctaaatccattcgaaaaaaaaaaataccaacccctaccttatacaatcccctcgcccgaacgtcataaaaaaaataccccctaaacgcaaacccccgaacgaccgaaacctcgcttctaacgaccgaaaaaaatcgacaccgtaacactcaaacacacatacaccaataccccgaacccgctaaaaacgctaaactccaaacaacgaaaaacgaaaatcctaaaatcgatcctaaatacattcctccactaaaccccgtcgaaaataaaactctcgaaacatcgtcactaaaaaatcctaaaaaaacactaaaataaacgaacttcttcaaaaactcgatttccactaaaccctcccattccctcctctcttcgtcacgcgataataataataataaaaaatacaataacccctctaattcctaaaacaccccttataataaaatataccctcctaactaaacccactccttactaaatacaaaaacaaaataaaaaaaaaaaaaaccgataaatttaacttctaaacaacccacctctacctctcctaaaaccacactaaacttatctataaaataaacacaaataaaataaaattaaccaaaatctcttctaacaaactaaaaatctactaaaaaaacgaactcgctccaaaactaacccccccccacgcctcttaacaataccacccccaaaatataaaaaacctcaaacaaatataaaaaattcttatctatataaacaattttaatcatcgcaaactcataatatcaacaccatcccgttaacacgatttctctaatcccgattaacaaccctcccaaaacctaaaccttacaaatctaaactaacttaaaacttccaccccaatcccaaaaaaaacccgcgaaacttaaaaaattccaaaaataaaaaacactccacaatataaaaccctctaaaatacaacctaaaaacacaaaaatactacctctcaaacccccatataaaaaacccgctaaaaaaaaaaaaaaaaaattaaaaaaataaaaaaaatacgaaacctccgaaactaacaccctcccccactcacccaaaccaaaaataaactaaactcaacatcctcatctcctcgccctatcattaccgccactaaaaaaataaaaacaaaatctctaccttctctacacacgtacgtaatctatataacgtaaaatcgcactaaaacccgaataaaaacatccccccgccccttcgtcatcgtaaaaataccgtaaaacaaaacgaacaaaaaactaatcaactcccgtcccgacccgaaaaaaacttccaaaaaaaacgaaaaaaccattcgccattccaaaacaaacgcgaacaataacccacgactctaaacccgaatctatatccacctctaaatacccaattcccgccaccatattattaaaaacaaaaaaacacaactacccgtaataaaaataaaaaacttaaatccccattaaaaccgacaaaaccccgacgaaaccaacgacgatactaacaacgcgaactacgtcaaaaacgaaaccgaattcgtaaaacgccgttacgcacaaatacgcgaatcaacaaaaccctaatacctaaacacccccgaatacgcgcgacgtaactaccctaataccccaataaaaacataactacgcgccaaataaaaataaaaacgccccctaacctttcctatacccgaaaccaaaaccgctaaaaccgatatactacaaaaatccgaacacaaactaaaaaaaccgacccgtacgttaaacgcctcgaaaatactccgaatcataataaatactaaacgaaaacacgaaaaactatatacacaacaacaaacaatacacccgcgctcccgtcattacatcgcgccgccttcacccccgctacgataaccactccgcaaataaacaaaacgaactcaaaaaaataaaataactttcccaaaaccacacaacgaaccaataaaaacaccgcgcacgatccaaaaatctaacccttcctaaaaaataaaaaaaaaaaaactcgacccgaaaaaaccaaacgacaaaaaaccccgcgtcccccgccacgaactaaaacgcaaaacgacccgccgtatctccacctacgcgcctcgaacttcgctccgctctaaccttcgaattaacaccgcttccttccaaaaacctaaaaaaacccgaacgaatcaaacgcctcccttctccgcaaccccataccctacccgcaacttaaactcaccgcccttctcgaaataacgatacgaaaaccgaacgactccactatacaaaccccgaaaaataaaaaaccgaataaaaaaaaaaaacgaaaaataacgaaataaaaactaaaaaacgaaactaaaaactaaaccaaacttccaaacgccaaaaaaacgccaaataaaccgaacgaaaacgaaaacgaaaacgaaaaaaaaaacgaaaccgaaaccgcgaaaaaaaaacgaaacgcccgaaaaaaccgcgaactaaaaaaatcccgaacgaccgaaaacgataaaaaaacgaaaccgaatcgcgtccgcgaaatcccgaacacaacgaaactaacgccactatcgaaattccgttccatctaaatcgaacaaccccgcgaatccgaacgccgctaccctcctctacctactaaaaaacgcaaaccgaacaaaaataaacctaactccaaaaccccaacgaaacaaacaaaacccgaaccaaaaactacgacaccgacttcccactacccgaccgaacgacgcgaaacctaaaaaaaacgaatcgaaatcaaaacgccgctacgccccgaaaaccgcgcttcccgcgacctataccgaaaaacctctccgcaaaaaaataaacacgccctatctccccgaccccgactcgaaccgatcaaaaataaaaaaaatcacgctcgaaaaaaaaaaataaaaaacgacgaaaaaacctcgataaccaaaacaaatacacgcgcgcgcgaaatcctcgtacgccgcgaaaaaaaacgaacaaaaaaacatcgaaaaaaaacaaacgaaaaaaaccgcgaaacaacgtaaacgaaaccccgaaaacacgaccgaaactacgcttcaaaaatatccgccaaacgccttcccgaacgattaacgaaacccgaaaaaacccacaactctaacctaaaacgccgtcgttccaaaaacctctacgcctctctctaacgtctatctaaaccctcaaaaccccgaaatcaaaacgcccatcctcttaataacgaaacgcacctttcgcgtaaatcgaacgctacaaaccaaacacgccgaatacgtcaaaaaaatacccgaaaaccaaaaacaacgcacccgaaaccaaaaaaaaaccgctacgcgcaacctcccaaacactacaacgaaatcgaaaccacgtccgcgtaaataactcgaactaaaaaaccgaaaaacgacgccaaaaacccttcaacccttatccgtcctccctaatatatcctcgacgcgatctaaaacctccccaacgaaaacgacgccccgacgccaaataccctaaataaaaaaacccgaaaaaaacaccacgaccaataaaaaaaaaacgaaaaccgaaaaaactacgtaaaaaaaaaaaaaaattaaaactaaaatcattctacaaaaaccacgttaataaatttaaatacgaacattctaactaaataaaaaaaaatttaaaaacaaaatacaaactaaaccaaaataaaaccccgattaaacgtaaataaacctacaaccaaaacaaccccaaacaaaaattcaaccgctaaaaaacacaaacccctaatatttcttaataatcactttttaaaacattttattataaaaattttcaaatataaaacaaaatcgaaaaaaacttcaaataaattcccgtatacccacccctatcccacaattcttaaaccgtaaccaatctaattccctctattaacctccttttatttatactaacttatttttattaccttattataaaacaaacacaaacacattttcactaaatacataaatacattttcactaataataactttttttaaaataacaatacgacttaaattacaattcccaataaatccacactcaaaaacaattaacttttaatattaaaaatacaaacgtatttccatttaaacttcactacaatatccaaataataaaaaacactattacttttataataaaaaataaacaaaaactcaaccccacaactccaaaacgtaacgaaaaccgtttactccccaaacgataaaactaactcttcgaccacttaactattctcgacgcccgatcctatatatccttccgtacataacacgcaaaacttaactctataaaaacaaaactatcccctctccgacccacctataaaaaaatatccacctctccgtttccctccaaaacaatcattcattaaataatccgccttaaaaacctaaaaatcgacgaatccaaaaaacccaacgaacgaatttccgataactaaaacccacttcccaacgaaaccctaaataaacatacccactctaactaccaaaaaaacaaaataaccgaaaaatataaaacatataaatacacaaacgaaaaaaaacaacaacaacaccaacgcaaaaataacccgctaac

SEQ ID NO: 158 - DNA, 2194 bp, Artificial Sequence
  Synthetic
ttaaacccaatattcaccactaaaacatttctatttaccttcacaaaaaaaaaaaaaaaaaaacatattaaaaccaacgctcacaaaacaaaaccaaacgaacctcactaaaaacaaactaaaaaataaacgtacgctatccgtaatactaaaaacttaaccgacgcgaactaaaaccgccaccgactacctcgaaatctcgccgaaccttacctactccgcgccctaaaaacaaatcttacaaataaactaataatactaatactaatacccaacgcgctaatcgccgccgccactactactactacgactactacacaccgaacgcgtctcgaactaatctccgacgccccgccgctcgcgctcgccgcccgcgccgacctcaaactccccgaaaccgcctttcgctactaccgcctccgaaaaacgcctcgaaccttccccgaaatcgccgaccgccgccacttcctaaccgacgaactacaaaaacaaaaacgaaaacgacaactcgtccgctcccctacaccgcgaaaccacctcccctaacgactcgcttaaccccgcgacgcgctcgaaaatctcgaaaaacgcgaacaacgacgacaaataacgcgaaaccgcgaaaaccgaaaccgactctcccgacgacgacgtcgacgacgacgacgaaaaaaattacgaaaaaaactcgacgtccccgctctccgccccgcgacaccgactaccgccgtaaccgccctcaaaactcataattataccgccgccgccctcctaccgacccgactaacgaaccgaactctaactacaaaaaaaaaaaacgcgaaaaaaacgaaaaaaaaaaaaaaaaaaaaaaaaaaaaacccgaacgcctaaaactaaaaaacgaaacgaaaaaaaaatacgaaaaattctacaactacgacgacgacaaacgcgaccgttcgataaaaccgccgactcgactctaataaaaacgacgccgaattcgactacgcgtaaaaaccgcgccgcgaaaaaaaaaaccgaaaaaacgaaaaaacgaaaaaaaaaaacgacgcgacgaaaataacgaaacgcgaacgcgaaataaactaaaaacgcgaatcaataaaacgaaattcgaaattcgactccgaacgaacgaactaaaaataaaaaatccctcaaccgcctccacgaaccgaccccgcgctcacgtcgattccgaaacgaataacccctctccaaacgacgcaacgctacgactctcgtaaactaaaaaataaaaaacaaaaaaaaaaccgcgaatccaaaaaccgttactaaataaaccgataaaatataacgcgaaccgaataaaacgcgacaatctaaaccgaaacccgcgtaaacttaaaaatacgcgaaacgaataccctaaacgcgcccgaactaactaaacaataaaacgaaaaaaaacgcgaaacccgaaactataaccgccacttccaaaccctcgctccccgcgcttaaaaccctcaaaaacactctcaaaaatcctcgaaaaccttaaaacaaaaatctctaaaacctatatcctctccctatctatcccgccctcgaatccctatatcctcctcacccgctccctcctacaataaacatcccgaattattaataaaaatcttaatacctaaaaaatcgaaacttcgtctcctaaaataatttatactaataaaccctaacgccacgttctataacttataatcactttcgtcgttaccgcataaaaaaacaaataacaccgccccttaccctaaaaaaataactacaaccttccccgaatcttaattttactcaccccgaaatcaatttctcgataactccaccctacaaaacctctataaaactcatcttcaaaacaaaactaacaattttctttctaaaaaaaaaaaaaaatccctcacctacaaaaaactaaactaaaaatcgtacacatacaataatttccaaatccgtacaatataaaatcataaaacaccgaatttatatacgacaatatatctatccgaattttcactaatataacgctttcaatctttaacacaaaaacacaaaacaatcatttttaacaacgtttcataaccgaaaacccaaaatcaaaaaaacaaaataacttattcaaaattataaaactattaacaaaaaacttctatattccctaaccaaattttct

SEQ ID NO: 159 - DNA, 1958 bp, Artificial Sequence
  Synthetic
atactaaaccacgctaaaaatttataactaaaaactaaaccctcgtcatcgccacgtactctatcaataaaacaaaataacccataattactaaactccgatcaattaaaaaacaaaaaacctccgctccccactactctataaaaaaaacccaacaaaaaaaccctaccaacttctaactctcaatctacgcgaaaatataaaaaaaaaaacccaaaacctccgaaacaaaacacaacaactacaaaaacaaaaccaacatatcgcccaacttcaaacttcaatatcacttcattctcatcttcctaacgactctaaaaaaaaaaaaccgatacctaaaaataaaccacccctaaactaacccatctcaccttccttacgtattaaccctaaacgacaaacaaactacctctactcgcaaccttttaaaattcgctatttcttccctctctactaaatactatcttacccctaatttcccctatcctattctccctccttacctctaaacatcccaaactacctacctaccttcctctaaccgctaaaaataccctaacctaataaaaaaaatctaaaaccttcccgtcttctccgaacacgaaaaaacattacccctctctctttattcgctatactaaataacaaaaaaaataccccgctcctaatccccttttttatccccaacccttaaacttttccgaactaacctatatttcttaccaactaaaaaaaacgacgaactacgatcctttcctactcttcaacgccaacctaaaacgaaaactaactaaaaaacaaccgtaccgccgcgctctacgtaaatcgaaactacccgactcgcgaacgcccgaaacgcgaaaaaaataaaactctatacgaaaaacaaaaaactcgcgaacatctcgaaaaaaaaactaaccgaaaccgccaaaaacgcgatccccttaactaaaaatcgatccgcgaaaacgcgccaaaaacgaaaaaaaataacgcgcccgaaacgcaaaaacccaacgcaacctaaactaaaaatcgaaacgctaaacactacaaaacccgaaaataaaacgcgcgaaaaacgaccgcccgaaaacgacgctacgacgactacaacccgaaacttattaccccataccctcctcccccaaaatacctaaacatactaaacctccaaaaccaattcaccttcaccgccgaccgaccgcaactacactacgcaaccttcttcatcaacgaacccgaaaaattcattaccatccactacgaccaaatctccatcgactatcaaaacgacgacttcctaaaaataaaacgcccacgaccaaccaacctaaccgaaaaacgacacgaaaaaattaaaaaaaactaaaacgctacacccaacgtaaaactactaaacgtaaataaccaataacacttcttaaacacttcgctaatacccgaatcgaaaaaacgcgttcgaaaacccccacacgacgaaaatctcgcccttacccgaaaacctcgacccacacacatccaataaaactaaaaccgatacctctaaacgctaccataatttaaattcccttctaaacgctttcgaaaccctaaaaacttctaaaaacttaaactcgcaacgcgaaactctaacgcaatccaaaaccgaaaaaaaataattaaaacccaacaaactcgaatactaaaatccaccaaacgaaccgtcttaattaaaaaacccgccccacacgtctcacatctaaacccgcctcgctcctactaatccacgctcgacaaaaccgttccgaaaacacaaaaactatcttcaatacgcgaataacccgattttaaaacaataaaaaactaactaccgaaaaaacatctaaattaaaataaataaaaaaacaaaacaaaacccctaaactcactacctcctcgattccaatcgtcaaactccataaatataacaaaac

SEQ ID NO: 160 - DNA, 9660 bp, Artificial Sequence
  Synthetic
aattttattctcctaacacttctaaacttcatttttactttaactcgaaattatttcaaaataataataaatatatattctaataacctacacacacgcaaccacatacacacctatttaatatatttattaaaaatactatactatacaaaaaaataaaatacacaacaataaaaaaaaccaaaatataaaacctacttccaaaaaaacaatcgtaaccaaaaaaaaatactcctctaaattaacgaaatcccctaaaacgcgaccttataaaaatatttaaatcccaaaaaaaaaaactcaaaacaaattactcttcgtaaacttcccgaatcctcaaattcccaactcttcaacccccttcccgccctcccgtcctcttctctttcttccccaacaactatcattaattaaaaaataataaacccgctaaatcaccatctccattcaccgccgataccccgaaactactacgtacaaatacaaaaacctctcgaacctctaaaattaaaaccccaaaaatcacgcgccttaatactactcccacaccccaaactaccaaccaaccttaatattcttcaatccctatattacttaataaataaccgataaaaaaatcttcttttaaaaaaaaaacgtcccaaacgaaacctttaactaaataactataatacaaatcgcaatttcaaaaatccctccgaaaaaaaacgccgttaaataacaacttccttccaacgccgcgtcccctccttatcacgacctacgaacgaaaaaaaaaaaaacgaacatctaaccctaattaacaaaatcaaaccgaaaaacctcaccaaaccctaacaccctacaacgtaactaccaaataaccctacgaaataacattctaaccaccgtatccgaaacccgcgctaaccaataatatatataacgcaaaacccgaaaacgcgcccgcgtaacaaataccgtaacctacgccgtaaaatactccgctataacctccgtaacaaaaataaaacgacactactacactataaaaactaaaaaaaacaaatacgccgaacccgaaacccaaacaaaacttaccatcgcgcaccaaaacgaacacaactactcgacgcgacgttaaaaaataaccaaccaattccaacgacttatcctaacgctatctcttacatttataacgtcgattctaaaaccaaatcttaacctacgtaaacgtaaactacaacgcgctaaccaaatactcgcgctcgaacgctaacaaataccgctactacttaaaacgccgctccaaaaccaacacctacgcctacgaaaaaaacacgcgcgacttccgtcgttaccgcgccttaaactactccgaacgaccaccccgcacgctatcgccgctattaccaacgccgcgctctaacaccctaatcccgccgccgaaaaacgaaaccgcgttcaaaccgaactctaaaaacacaaaaaaaaacacatcaacgcccaacctaaaactcacctaaacgattcctacttaattttcaaaactttcctctccctaacaaccttcactctacgcaaactcctcctccctcctactacccccgaaccctatcccaaaacgaaccctatcccaaaactatctccatattttttccctccaaactctatcacctttatatattactactaaaacataaattataacaacctataatccctcgtatatacgtccttttcctaaaaataaactctaaataaaaaattaaaacaactccatttacttcttaaaatatatatacataaaccgaacgcaataactcatacctataatcccaacactttaaaaaaccgaaacgaacgaatcacgaaatcaaaaaatcgaaaccatcctaactaacacgataaaaccccgtctctactaaaaatacaaaaaattaaccgaacgaaataacatatacctataatcccaactactcgaaaaactaaaacaaaaaaataacgtaaacccgaaaaacgaaacttacaataaaccgaaatcgtaccactacactccaacctaaataacaaaacgaaactccgtctcaaaaaacaaacaaacaaaaaacaaacaaacaaaaaaaaaactatatatacataataaaccttcgctataaaaaacaaatataaataaaaatatctatcaaattaaaaaaactttaaaccctccttaaaaataaaaaaaaaaaaaaaaaaaaaaaaaaaaaaaaaaaaaaaaaaaaaaaaaaaaaaaaaaaaaaaaaaaaaaaaaaaaaaaaaaacaaaaaaaaacccgaatatcaaaaaaacgcaaactcgataacaaactctcaaaaaactcctatctacctcattttccttcgcaaccccaaacgcactacaaaaaacttaactataaatccaaaaacgaaaaccttaaatatctcctaccttcacccaacttttatacacactcttatctttttaacgcaacgctccaccaacaccaaaaatacatcccaactttcttaaatataatactttataatccaaattaacaatatataataaaaacaaaatatttattaactcctaacttttaactcaaaaactaaaattctctttaaaaaatacataaaattaaaatccacatttaacttcttaaaaaataacctctccctcctttaaaatctttctaacaaacaaataatataaacttcaccctaaaatcttcctttcacccaaaacgacccctacaactaaaactatacccaccaaaatacaacgttttcctcccttctcacttctctcctatctaaacttaaaccccgttttaccccaaaaataaataaaaaaaaaatactaaattttcataaaatctcacgccaaaaaaaaatcaaaaacataaaaaaaatacaaattcataacaaaacgaaacaaaactctaaataaaccaatacgattaaaaccaaactcctaatacccaaaacactaacacaatctcctaaaaaaaaataaaactatttaatatataaaatttaaaaacaaaacaacttccttcaaaaacgtctatactcaaataaactaaaaccatctccacttacaaaaaaaaaaaattctaaaaaaaatttattttcttaacgcatcgaaaaataacaactaaccttcaaccaaactacaaaacaaaaaaaaccccatttcaaaatcacaaatcatacaaaccctaaaactacacaacaccgcaaaataaaccaacaccctacgcccttctattcacaatcctaaaattcttaaaaacaaaataattaactacgataaaaaaaaacataaaacccaccgaaaatatcttccctatccgaatcgctaccgcgaccttaaactcaattcctaaaaccgacctcgaatccgacctcgaatccctcctcgacgacgtccactactaccgcaaaaaaaacgctcccaaaatttaatttccaataaaaaaaaaaaaacgccgcaaataccatcgactacgaaacgactataataatcgaaaaaacgtaatcctttatcaactcaacgaaaccgaaataatcgccgaattaactaaacaacgccgaaaaaatttaaaatccgctacaaaaaacctaccaacttcttcgcgaacacaacaaataatattcttaaacttctaaaattaataaataaccgacctacaaaccgataaataactccgatctaaccccttaatttatttctttcgttcgcgccttaaataaaaacgccataacacaatctaacaaaaaaaacctaccctaacgaacgctactcaaaacccctacgttaaaaataccgaataaaatactaaaatttaaaaaaaaaaaaataacgttttcatcccttaaaatatcgaacctctccaactaaaacctccgaatcgacacaacacaactcccgaatcgacgcaacacaactccccgccaaccccttcccttccaacgaactaaacacaaatacgtaaacctcgcaacccgacgcgcacgattacgcctttaaatacctacgacaaaactcgaccaaaaccgtatcccaaaacaaaaaaaaccctaacaacctttaacgttctccctaacgcgaaaaatcctacgccaacgaaaccttccacttcgaaaaactcaatcgccacccgatccccaactttcgtcttccaaaaaaaaaatccataaaaaaaaataaaaaaatcccccgaccaaaccaacaaaaaaaaacccaattaaaaataacctcaacgccacgaaaattccactactttaataaccacacgcttttacaaacgatttaaaaataaaaaaacaccaaataataataaaaaaaaacgataataactaacacgactaaaacactacgaaaaatttcgctataaaaataaaaaaataaaaattctaaacactataactacgaatataaacgatacgtttataaaaatcaaaaaaacgaatttaaaaaaaataaatccttaaaattcaaaaccttactttcgttaaaaatatataaaacacactaaatatcaaaaaaaaataaaaataaaattcgaaaaacctttttttaaactcctcgaaaaaaaataaaaaaaatctcgaacccaaaaaataaaaaacgtattttccccatacacccataaatcacgcccccgcccccacattccccccgtaaaaaacaaaacctaaacgcttactcacgtaactcccccatccgttccgcgtccatctccaaaactacctcacaaaaacccccaaaaaactccgaaccatccaactttctatcaccgccgccgccaccaacgttataaacctctaaccctcgcgactctacgtccattctcaaatactaaaaattttccgaactcttccgcacccgcgaatataacgaaaccgcgaaacaactccgctcgcgctccaatcgcaaaatatccttaaccgaaaaaaaaataaaaataacgaaactcaacaacatcccgaaaacgaataaaacgaaaccgaaaaaatccgaataaaaaacgacaccctaaacttcccgtcttatcgctacaaaccccgcaaacaaacccaaactctaaaacaaacgcccaacgtcccaaacaacgccttcctctaaaccatactaataaacccgaatccaaaaccgaataacgaaaccgtaaccccccattaattctcgcaaaaaccacgtactaactctacaccttcctcccccaacgctttcttcccgcgcccgtcgaatcctctccaatccccaatcgccttcctaaaaaatttaattcgtattaacgattctcaaaacaaaaaaaacgtaaaaaaacaatactacgcgcgacttaaatctccgtcaatacgatactaacgaaaacttccgaaatcaacctaaaatccacgaaaaccctcctaataaacatacgaaaattaaaaataaaatccttactatacaacccgatccgatccatccaacctaatcttaactcaactctacattcttataacgaaaaaaaactaaaataaaaacaactaaaacccgcgaaatttaaaacgcgcgctaaaatcttacgtacgcaaaaaccgaaccgcaaaataaaaataaatcttaacccaccaaaacctatattaaaataaaaacctctctctttaaacaaaatccaaatctacgaaatcccgacccgccacttcctaaaaacaacttctcaaccccaaaaccgaaaaataaatataaaaactacatataaaaaaccctcgacctcttaaaaaactccgcgaataataaaaaaaactaaaaaacaatctctacgacccgcgcaaaataatcaaaaacgaaaataaaaataaaaaaaaataaacttccgtcgcccttaacgtccccacgttctttttcttcctctcctttatcctccttaaaacgaattacctaaaacgtaccaacttatatacgcgtataaatacgacgcccgccgaaaaactaaaaaaatttaactcgcattaaatctccaaaaaaaacccttaattacgacgaacaccgaataaataacgcaatataacacaaaaattaccaacacgaaaataacgccaacaaatccaacccctactattactactcctaaatcaaaaaaacgtaatttaatatcaactattccaacactaaatcgaaaaaaatttaaaaattatattttaatcacaataaaatctcattaaaattcttttaaaaatttcaaatcaaatatttctttttatttttctttccttcctttccctccctccctccctcctttttctttctttcttttctttctttctctttcttcctttttttttttttttttaattacaaaatctctctctatctcccaaactaaaatacaataaccgaatcataactcgctacaacctcgacccgaactcaataatcctcccacctcaacctcctaaataactaaaaccacaaatacaaccactaaacctaactatttatttatttatttattacttataaaaccgaaatttcgccatattacttaaactaatcttaaactcctaaactcaaataatatacccacctcgacctcccaaaatactaaaattacaaatataaaccaccgcgcaaacttcaaatacttcttaaacctacttacgtaaattcaatattacaacaactatttaaatataattatctcctaaaattcaaataaacccaactcatctccccaaactaataaatacaaaattacacacaaatatttatacaaaatttcaacacatttaatcattcaactacaaaataataatttcctatacataataacatttaatcttcacatcaaaactatacccaaatatttccaactactactccttcttcttcttcctcctcctcttcttcttcttcttcttcttcttcttcttcttcttcttcttcttcttcttcttcttcttcttcttcttcttcccctcctcctcctcctcctccttctccttctccttcttcttcttttattcaacgaaaaatccaaatcaaaaaaataacccaaataattcaccaaataacaaaaccaaactaacctctaaaccaaaacaatttaccctatctttctttctttttaaaatatacatcctttactcttaaaaatctcaaaatcttctaataacccatacatttatttaaatttttatttcctaataaaaatcatcatacgcaccttaaaaaaaataaaaaaaaattaaaaaaaaatatccacccaaaaacaacattttaataaaccttcttatctttatttctatatatataaactcataaaatatatatatttatatatatttaaatttacatcataaacatacccaattcatctttaaaattttttaaacaaatataatttaaaataaaaatctaatatttcatattattaataaaccgtaataaactatatttatatttttaaataatatctttttttatcactataaataaaactttattttataccttcaattttcatccttatcaatattttctattattttctcaaaatacatatataaaaaaaacatcaatcaatcaaatattttaatactcataaaacataccattccaaaacttcctaaaaaaacaatattatcgtattttattcccgaaaatacaaacgaatacctaatctacgaatatttacaatcatcaccgaacacccacaaaaaaccctaaacttaaaatatttccgcgataaaaattccgcctcgtccttccaaaaaatactaacgactcgctaaattctcgccttccaaatactaaaaaaaaaaaataaaaccacccgaaataaaaacctaacgccaaaaaccttccaacgccttatcaattaataaaattttcgtaacaaacaatcgcgctctacctaaatcgtaaaaaaaaaaacaccttataaaaaacttaacgtcgaaaaccttaaattcaaatcgtaaccctaaatccccaaccttacccttcctaacttactacgaaaataaaataaaatttttatacgtaaaaatactttctaacccctctcgaaaaataaaaacgtctccattcgaccaaaatccgaatctacgaaacaatttacccgccgctttccaaaaacaaactccccaacgtaacgaatccttattataaaaaaaatcgtttcgactcaaaatacgcgctcccgacgtaaacctaaaaataaaaatccctatcgcgctcgccccacccctacaaaaaacaaaaccgtccttctacaacaccgcgccacgcgacgaaaatttacaatatcctactttctaatttcaacttcttcgctcgcttaaatctctctcgatcacctctcgaaaaaacctaaaatccctcctccctcctcctacttcaacgccaaaaacattaaaaaccgcccgcccgccgaaaataaaaccgatacctcgaatctcgctcattccgaatcaattaaaaacgcattaacgactaccttacaatacaacaaaaactcgaataacaaaccacctacctaaacgctaacgcccaaaaaaaccgaaaaaaaaactaaaaaaaaaaacgacaacaaaaaaaccaaaaaatccaaaaactcctaaacaaacctccgtacaaccccaacaacgtaataccgaaaaatatatatatatatatatatatatatatatatatatatatatatatcccttactcaaaaataaaaacatcgtaatcacctctattcttaaataaaataaaaacgaccgtactaaaaatataattaatccgctttacttcactcaaatccgcaaaatattttaaacgcctaccgtaaaccaactctacccaaactctaaaaatactaaaaaaataaaaaatctcaaccttcttatccgaacttacattcttacataaaaaacaaacttttaaataattcccatacattatattaccttacacaatcaaacgaaatttatacactatatttattaattttttttttttaaaacgaaatttcgctcttttacccaaactaaaatacaataacataatctcgactcactacaacctccgccttttcgatttcaaacaattctcctacctcaacctcccgaataactaaaattacaaatacccgccaccacacccgactaatttttatatttttaataaaaaccgaatttcaccatattaaccaaattaatctcgaactcctaacctcgtaatctacccgcctcgacctcccaaaatactaaaattacaaacgtaaaccaccgcgcccgaccctaaacctctacttctccgtaattaacaaaaatcattatttcctatttctttcactctcctaaaacgaaaaaattccctccaacaaataccatactcaacaaacaattcctaataacctcgcgacttaataaaccctataaaccgaaataaaatcaatcctctttaacccaaatacaaaatccccgttaaactaaactaaacttaaactctaaatctattatcacaactatatccccaaaacacgtaataaatctttaacaaatatttattaaaaacaaatatctatcctacttaattaaaaataattcctaaaatactctaaaaactatcccaaaaaatatctaaaaaccatcaataataacaatttctactttaccacgatcccttttactaaaa

SEQ ID NO: 161 - DNA, 1451 bp, Artificial Sequence
  Synthetic
tttttctttaaaaaaactattcttttccatatcaacgatttcattttaaaaacaatcgctaaaataatctcaaatataaaataaactaattaaacacacgccacttaaatactaaaattaactaaaactcaaattttcaaaacaaaacaaaaacacaaatcaaccccgctaaattactactactcctaaaataacccgaccccatcacgaaaattcccaaataactacgaacgtcaacgcttaacaaaaacgattaaacgattcacaacactcaaaaaattatataaatacccgacccgaacgaccaacaaaaacaaattcaactccccgaacgcaaaatcatctccaacccgatcctcgctccccctaaaaacccaccgctcgtctccccgaaaactacctccgtaaaaaaatattcgcccccaaccgcgccccaactttaccttacgtaatcccaaactttccctaataaattaataaaaaaataaatctaaaaaacaaacgccccacctataaacttaaaactaaattacatttaataaaacgcttaacccgcatccccctaaatcgtttataaataactaaaaaataaaaacgcccctctacgcgtctcccccgacgacaaaccctaccccacgccccccatcccaaaccaaaaacaaaaataaaaaaaacgaaaactccaaatccacgccccgaaacacaaaaaattaactaactcctaacgaaacctaaaacgcccacatccacgacaaaaccacctataaacctacaaaacaacaacgaaatacgcgcaaccgaaatacgaaaacgaccgccacaacgactacaacgacaaacgaaacgcaaccgcccgcacttaccaaaataaaataaaaaaacgcgcacacgacgaaacccccgaacgcgacccgccgctccaatccgccgaccctccgcctacccgctctaaaacccgaactctaactcgcactaactaccgctctcgccaaccgaactaaattcgaaaaaaaactaaaccgctataaacccgacgctccgaatctaacgctacccgacccccgccgacccctccctctaaactatacgctatacgctaaaaacgaaaccgcaacgcgctcaactcccgaatcctttactccacgcctcctaaacgcaaaaacgacgctaacaaccgtctcttaaaaaatttctccgcaatataaacgaaaatcaaaatttacaaacgaacaaaaacaaaaaaaaaaaatcgtacaaatcgataacgcgcctaattcacaaaacctaaaaatactatcaactttaaaatcatcttatactaaacaccgcctctccaattatctacaaaaaataataacaatcgcgcttttctttaaaaaacgatataatatataataataaaaaaaacgttcaataaaaaaacacgaaaacctacaaaacaaactattaaa

SEQ ID NO: 162 - DNA, 1242 bp, Artificial Sequence
  Synthetic
ccgaataaaaaatataaactttaacaaacaaactaaaatactttcatacattttatactaaaaccgtaccaactctccgtaaaactctttacctacgttcgccccatcccctaattcgcattattctcaataataaaaacctccttacctatcactaacaccaattaacttacaccaaacttcaaaacgccaaaaaaaaaaaaaaatcactcttccaacaacgatcatttaaaaaaaaaaaaaattaataataatataaaactccctccaaaattaacctacaccgtaatataaattaattttctttataatctaaaattaactcgcaattaactaattaaaaaataattactaaaaaaccgaaaccttaaaactttcgaatcctacccgcctttcatcatataaacaaacgcatcaaatttaaaactttcccataattattatactaaccttaaaacgcaacctctccatttacatttaaaaaaactaaatattaaacaaaaaaaaaaatactctttttaaaaacctaaaaaaatatccccgctcgaaactactccgccatctaaaccgcgaactaaacgcgcgactcccgcccccaactccttaacaaaaacgccgaaaaaaaaaacgccgcgaaaaaccgtcatcttattaaaaaaaaatacaaaaatacccccctaaaactaaataaacaccacttccacactcaaaacgaaaaaaaccgaaaaacgtaaaaacgacgcgccaaaaacgaaacgtccctaataacaacgcgtcccgaaaactctcccttttcccaaaacgcataccaaaaacaaactcctttacaaactccacgaaccccattcgcgataaaaaaaaacgttccaaactacaaaataaaaaactccgatatatatataatatatataatatatatatatacgcgcgcgcacgcccttaaaaaaaaatccatcttttataaatactataacccacttttaactaaatctcgtaaaatactcaaatacaaccgatataataaaaactaataacatcaacacactttcgcgcattaatcttaaaataattaaaaataaaaacaataaactataaaattttattaaacaaaaaaaaaaaaaaattccaacctcccatcaacctattctaaaaaaatccaaaaaacccatacatccctctacctttccatacaacctacacacaaaacatatctatcgaaaatcacgaaaaaaatcattccta

SEQ ID NO: 163 - DNA, 1206 bp, Artificial Sequence
  Synthetic
aaaaacaacactcatttcttcttatcatttctaaactacgaaacaaaaaactttaattttaaaactaaaatttaatttatttttaaaaataccgaatattaccaaacaacgccaacacgaatataataataatataaaatttacacacacgcaactaaaataataaatttaaaataaaaaacactatatacacgtataatatatacatacaatatcaaaacattctttactcctaaaattttctctactttaccaaacttcacgccccttttaaaaaaaaaaaacgaattttccaaattataaaaatacttttcctccatccaaccacaccctcttccgaaccaatctttaataaaacctaaacgatataaaatcactaaaaaatataaataaaaacgtttttactcatcttaacccaaatctctaaaaaaacctttaaaatcgctctaaaacttaaaaaaaattccgtcctaaaccgaaaactaaaactctccgaacaaacgctaaacaactctcacttatcgatatccaataaaccttatataaaataccaaaataccccaacaaaactttaccaacacctaaaccgtaattatctcctaactcctaaaatccgcgaaactctaaatatacctacaactcctcccgaatcctacaaaccacccttatccctcttctcccgctcaccccccgacccccccatctcttttactattccgaaaaaaaccacgcaaaatacaacccgaacgcgcccccgaaaaaaacccgcgacgcaacaaccacacccccaacctacactaacgcccaccccataacttcgccttcgcgatcgcgaacaaaaaacgcgcgtttccatcgattaattaaaaacgaaaatcgaaaaaacccccgcccgccgacgaccaacgcacaacccgaccaaaaacgccctccctaccgccgccgaccctttaatcgcccgcgacccgcgacccgcaaaccacaaccaacgctatactaaacaaataacgacaacgaaacgcgacgcgctcaaacgcacgaatccccgaaccgcgacgcgctccccgccacgccgcacatcaaaacccgaccgaccgacgaacgccttcattaacaacctaaaattataatattatattaaaaaacaaaactactccccgaaaaaatatatactacatatctaacaaaaataaattaaattattca

SEQ ID NO: 164 - DNA, 2308 bp, Artificial Sequence
  Synthetic
ctaattctaaaatacaatataacattaacgaatataataaactccgacaaacctaaaacaaaaaaatctccgataacatccctcaataccgaaaacaaaatttcccgaccacttccgtcgattaaaattctcttaaacattaccctaaatctattttcaaataaaaaaaaaaaaaacccaaaccaaattaaaatctaaaacaactaaaacaactatcgatttccaacttactttcaaatctaacaaaaactataaaaatcctaacttctccgcgatatcaaccacgacccgcgacgatcacgaaaccacttcaacacctcgcgataaacaataacccccgacgtaacgcgaattccctacgatccccacgcgaacctaaacgacgaatccctccacgactccgtccaaacacccgaaaacacctaccaaaactacgaaaataaacgaccgaacgtccaaacacgaactttctcgccgaaacacgactaccgaactccgtcctaaaaatttaaaacgacgaaaaccgccgaaaaaaaaaacgcgcgaaccgaaaaccaaaaccgaaccgaacaaaacgcgcccaaaaacaaaaaaaacgacgacccgaaccgaaacgaaacgaaaacacgttacctacccgtaaaacgcgaaaaaaaaatacgcgacgacgacgacgacgaaatctaacgcaaaaaacaacgacaacaacgaaaacaccgactcgctcgaaaccgaccaaccgaaaaaaccgaaaaacaacgaaaccgcgaacgacgaaaccgaaacgaacaacaccgaaaaaaccacgacccccgcgctaaaccgaaacaacccgaccgaacccgacaccatccaaacccaaaacgacgacgacgacgactctacgtccatcgaccaaaaaaaaaactaccgccgccgaaactactactactacaactaccgccgccgccgctaaaacctaacgcccgcctacatcctacatccccgcgctaacgacgacgacgaaaacgacgaacgcccgaaatactaaaccgacgacgacgcgactccgaaacgaactcacttaactccgcgacgcgaccgctcgctccgcccctaccccgcgcgaaaaactcctcctcctcccgcccgcgccgccgccgcccccgaacccgacccccgccccgatccccgcccgcccgcgcccctccccgccgaaccccgccccgccctcgcgctccccgaccctaaccgaaatctcctccccgctacgcccctaaacccgaacgaccgcgaaaaacgtaaataaaaaaaaaccgaaaacgcgcccgactaaactcccgccgataccgaccccctaataacctctaaccctctaaaacccaacgcaccgcgaaaaaaactccgaactccgcactcaaaaacaaacgaaaaccgaaataacgacgccgaaaccaaacatatcccgctacccaacgcgaaactaaaaacgcaaacgaaaaccgcgcgaaccgctctctacctaacctcctcgaaatccaaaacgcgaaaaatactaaactacgaatccgacgcaaacgaccccgaacgctactaaacgcgaacgaatttctattacacaaataaaccaaacaaattcgtaaaacccccgcgaatcccctaacccgaaaattaaaccgcgaatacctaatctaaaattccgcgccccacaataacccgcgaacctcgacgactcctaccctacgccctccaaaacaccaaacaaataaccccaaaacctttacccacgacaccccctaaataaaacaaaaccacccctctataaattcgtcctaaatacccccaaacacaatctatactcctccctcccccaacacacgcgacctctaacgctacacgccgaacttcctaaaacccaaaacgaaccgaactccaaaaaccctcgctccgcccgaaccacacgcaaacaacgccgaccgaaccgaacctaaatccgcactaaccacgcgcaaccaccgccaaacgacgctaaccctcccccgctataaaccctacttcgaaaaccgccaacctcctccccatcccgaatccacaacttcaatacaacccctaacgacgcgcgctaacaactaaacaaaaacttccgtacgctataaaccccttaaatcgctaaacccaaaaaacccaaacacaactaataccaacgcgaaaccaatattattaaaaacatttaaaaaatctttctaaatacaaaaaacaacattataatccatacatttaaaaacttacataaaaaaccctccctaacctaaataaaaaaacaaacacc

SEQ ID NO: 165 - DNA, 2201 bp, Artificial Sequence
  Synthetic
tctccctttctacatataaccccgaacgaaaaaaaacaaaacttaaaaaaaaactataaaacccaaaataaaataaaccctaaacaaaataaaaaaactcacacctacaaactacgtacaaccgaaaaactaacgacctataacttatcaaattcaaatataatacaaataaaaacctaacaaacatttaatttaatttctaaaaccgttaaaaacaaaattaaattccaaataaacgcgattccaaaataactaaaattcccactctcaaataaaaacccttcaaacaaaaataaaacaaaaaaaacttcttcgccgcccgccctacgatatataccacgaaaaccgctaaataaaaacgtccaaatataaaaacgttcctccaacatacgtaaatatcctaaaaactcacgaacaccgattccctcttccgaacccaaacgtcctaattcccgtcccccttaaaataaaatcccgttttcccccaaccaaaaaaactccgaaaccgcattcacctaacttaactaccaacgcccgccttcctcgaattctcaaaacaaaaaactataatacattttctaaaaataatttataaatataaaaaaaaaaaatcctttcaaaaattttaatcatttaacaattaataattttcatataacgtactaattttaaaaaccaactctaataaactaaaacgccctccccttaattctaaaacaaaaaacattttaaactaaaaacgctcacaacccgatttcctatcccgcgtaattcataaaacttaccctaaacctaaaaaaccgataacgaaaacctcccctacaataaaataaataaaaaacgccacacgacgtctcaaaaaaaaaaaaaaaacgacttctattccgaaaaaaaaaaacgataaaaaccgccgttctcttaaacgcgacctctactaaaaaacgacgaaaatcgcaaaaccgaaaaaccgaacgcgcgcgaaaaaaaaacccaaaccaaaaaccgctcgcctcgaaacgaaatctctaaaaaacgcgcaaaaaaaacgcttcgtaaatacctaccgacctaaaaaatatcgctaatctctacccccttcgccaaaaccgaaccaaacgaataaaaacgcgacctaaacccctacgataattccccaaatataaaatcaaaacaaatcccaaaacgcgatccgcgaaacttttaaaaaaaaccgcccaaaactaaaaattccgacgcaaaaacccgaaaacccgcgaaactataaacgcaactaaaaataacgaaaaccccaacaaaaaataaaaactcgaaatccgcaaaaacaactccatccccgacgctaaaaaccgcataattcccgcaaactataaccaatcgaaaaccgaactccgcattcttcaaacgttcctaaaccccctcttaccccaacgtccccctcccgaacaaccccacaataaacgattttcccgcactccgcacgaatccatccccataataaactaaaaaccgaatctaaactctacgctacccgaccaaaacgtccaacgaccccactaaacgacgacgccacgcgaacgtaaataaaaacaacccccgaacccgccgaaaaaccctcgcgaaaaaaaaaaaccgaacaacttaaaaacgccccgacaaaaaaaaacgtacgactaaaaaaaactaacaaataacacccgacccgcaacctaataaacaaaataccaactatccgcccaatttcctatccttacaataaaactataaaaaaaaacatttaactcgacaaaaaactcaaaactaaattttacaatttaattttacaatttaattttaattaaaaacaaaattatctttttttaatatatatatatatataaaaacaacaaacgaactaatttaaaaactttatatatatacatatatacgtatatatatatacatatatatatatatatacatatatatatatatatacgtataactaatttaaaaacaaataactactcactatataaaatttctaaacattcacaaaaattatttaaaaaaaataaaaaccgaacacgacgactcacacctataatcccaacactttaaaaaaccgaaactaacgaatcacttaaaatcaaaaattccaaaccaacctaatcaacgtaataaaacaccttctctaccaaacaaacaaacaaacaaacaaacaa

SEQ ID NO: 166 - DNA, 3047 bp, Artificial Sequence
  Synthetic
acatcgaccttacactaaaacttcatcctttcactccctaaacaactaacaaaacacgaaaacaaaaataaaaaaaaaaacctccctccgactttaacgccttactccctcacgaaccgctttcaaatatacgaatccccttcaaacaaaaaaaaaaacaccactaaaaaaaaccaatcacccccatttcaatatacaaataaaaaacctaaaattaaaccctttcctccatcgactaatctctaactataattaaacactcccgaaaaataaataaaaaaaaaaccaaacccccaaaattaaaacaataactaaccgaacgaaactctaccaaaaacgaatctaaaaactccaaaatacctaaaacgtaaacgaaaaaaaaaaataaaaccgcgctcaattccctctctaccgctacgcgtaatccgcgctcgaactcgcttaccccctctctaacccaaaaccccattcgcaacgcctaaactaatcccactcatctacgccaaataaaaaaaaaaaaaaaaaaatctaaaaatataaaataaaaaaaaaaaaaaaaaaaaaaaaaaaaaaaaaactaaacacttcaaaaatcaaaaaaaaaccacctataatcacaaccatcgtcacgaccccctaccgcgcccacgcacgcgtaaataaaaccaaactctaaataccacctaaaaaaaacatccattcataatccgaatctacccccgcctataccttttctcctcgtaactcccaccccccatccccgaccccgaatctctaaactaaatttcttaaaaaaaaaacatctctaacaccgtttatcctaataaaatcaaatccctactttatccaactcaactctaaataataattattaaaaaaacaaaaaacaacaaaataaatcaaaaaaataacaaaaaaataaacgcaacgactcacgcctataatcccaacgctttaaaaaaccgaaacgaaaaaatcgcttaaacccaaaaattcgaaaccaacctaaccaacataacgaaacccgtctctactaaaaatacaaaaattaaccgaacgtaacgacatacgcctataatcccaactacttaacaaactaaaataaaaaaaacgcttaaacccgaaaaacaaaaattatataaaccgaaatccaaaaataaaaaaaaaaaaaaaaaaaaaaaaaaaaaacaaaaaaacgaaaaaacaaaaaaaacaaacaaacaaaaaaaaaaaaaaaaaaaaaaaaaaaataaccactatttctatactatcgctatactattctcgacacacctaaacccaacctttccattccttacctatattttcttccatttattcacgtctaaaaatatattcaaataaacaacaatctaaaatataaatatattatttaatctacactaatatttaacacaactatttataatactcaaaaaattataaactacctatttcacaataatcgattaattataaatcataataattttttttttttaatacgcaatcttactctatcgcccaaactaaaatacaataacgcgaccaccgcctccctaattcaaacaattctctacctcaacctcccgaataactaaaattacaaatatctaccaccgcgcctaactaatttttttatatttttaatacaaacgaaatttcaccatcttaaccaaactaatcttaaactcctaacctcgtaatacacccgcctcgacctcctaaaatactaaaattacaaatataaaccacgcgcccgaccaatcattttaatcgactttaataaacacgtttcaaatcattttaccgaaaaatactatcaaaataaaatactcactacacactaaataaaaaaaaaactatttacaaaacaatttctaataaaaaaaaaaaacacgtaaacataatctaaataacgaaaactaaaaaataattatctctaaataactaaatactaaatataaattaaccgcaatattatttttataaaactaaaaaaaaaacgttattttaaatatacgaaatttactcctctccaaatccactctccctttcgcctccctaaaaattatcaaattcaaaaaacgacccgaaattacaaaaaaaacgccgacgtcactaaccccaaaaactcgaaacgcgcgcgccgcaaaaataccgactacgaaatcgaattaaaactaacgaaaccctcgacctctaccgaaatacgaaaaataaatactacgaacaaaaaaacgaaacttacgattcccaaatccaaaaacgaattaaaaacgtaaaccgacgtccatatattctacaccccttctcgcccgatacctctctcaaaacacgttttccaaaatatattaaattcgaaaatcgatcgaaaatttcaaaaccaattaaataccctctaatataaaactccgattaaacccgaaaaacttcaaacaacccctctaaaccctcgaaaatcttcgccgcgataaccttaaacgtcccctccccgaaaaatctccctaaaactttcacaaaaaacgaaaaaaactacgctaaaacctccgttcccaatacccctaactaataaaaaaaaataaccttaatatctaaaaacaaaaaccaaattaactcccacttaaaaccaaaaaacgaaaaaaaaacgaaacccgaaataactacgactcccccgaaacacaaccgaaaaaaaaaatcctaaccatctaccgaccccgcctaaaaacatctactcgaaaatccctaaaaaccgttatcgtattcctcaaaatctaccgtactatccctccgcccccaaaaccaacaaaacacccattctataattctcttcttctccatcgcccaattaaaaactactaaaaaccaaaaaaaaaaaaatacctttcccgaattctctaattcaaatacttatcaactttacacaccattcgacctttccaaaaaaaaccaaaaaacaaaaatatactaataacaaaaaataattaaaacctcgcctaccttacatattttattcataaaaactataacaataataatataatcct

SEQ ID NO: 167 - DNA, 3780 bp, Homo sapiens
ttgcaggttgacctacggtggtggtggtctaaaatcatatagaaaccagagatctgatggtttacctgaaaatgcgaagtaaatgcacatactcagcatctcagacgatcgaaagctcaacgggtgaaagctcagggggttaattttgcgattaaggacatcttggaaaagtatgtaaaatccctggtccacttaagtattattcctattttgggcttttatttatttattttgagatggagtcttgctctgtcgcccacctcggctcactgcaacttcagcctcccgggttcaagcgattctcgtggctcgtcctctcaagtagctggggccacgcccggctaatttttgtatttttagtagagatggggtttcaccgtgttggccaggctggtctcgaactcctgacctcaggcgatccaccgcctcggcctcccaaagtgctgggattacaggcttgagccacagcgcctggcctattttgggcttttataccccactggtaaactgctttcctccaggttgaggttaaaacgacatcattttaaggtgaactgaagtctggaagtgattaagcacttggatccttagggagcctctccccgcccccatctctttcatgctaagataattaaaacttcggggccggggcattgtctgagtcacttcaattcaccagcctaatagatgcaaaaggatgtaagcatgtcagacacgcagagacagtatttgaatcaagcttaatagctcaagggagctgggccatttcctattttctttttagtgagtgcgatgttctctaaacaccgcggagaggcagggttcccggtgatggccttgccgagggtgctcccgcaacctccacctccagttctctttggaccattcctccgtcttccgttacacgctctgcaaagcgaagtccccttcgcaccagattcccgctactacacgccccccatttcccgccctggccacatcgctgcagtttagtgattgactggcctcctgaggtcctggcgcaaaggcgagattcgcatttcgcacctcgcccttcgcgggaaacggccccagtgacagtccccgaagcggcgcgcgcccggctggaggtgcgctctccgggcgcggcgcgcggagggtcgccaagggcgcgggaaccccaccccggccgcggcagcccccagccttcacgccggccctgaggctcgcccgcccggccggccccggctctcggcttgcaaagtccctctccccagtccaacccccggcccccacaggcctcggcgccccgccccgccccaggccccgccccagagagttctataaagttcctctttcccacctcgcactctcagtttcaccgctcgatcttgggacccaccgctgccctcagctccgagtccagggcgaggtaagggctggagtcgggcaggaggaggggtgtgaggctgataccagagaggacccggagcgcgaaccagaggttcgacctccagggcagcgcagggtaccccggcttcggagcgggaagggagcgcgccccgtcctggagctccgactcccaccccatctgcgctgagccggaggcgctggtttgggctccaaggcccgcctccttggctctgcccgagcctccccgcctgccctccgcgctcctgcgacggggtcgccacaagctggacgggatgagctaaccggactgtcggggccccaggagtggctgaggcggggccgtccaaggcacccacacaagacggcacaactgcctgcgagaaacaggcccggccctgtggaccccaatccgaggctccttcccctgctcttcgttcctaaggggcccaagctcacggcggcctccggcgcggtgctcacccgctggcgcaggaggaggaggagctccacatttgggtcgctccgagccttgcgtgcggtggcctagccggcctggcgcggtccctgcctcccaggctccgcagctgtcgtcgccctctcccgcgccctccccgcctccgctctcccgggcctgctccggggtccggcggacgctctgcgcgcggaatcccccgtactggggctgcagcccccgcgtctgcgccacttgtcgtttgcagagcccacttagtgcgcgctagctgggcagggataggggtcctattcggggcgaagggtctggatgcgagcagagaaagcggagggtggaggaacccggggctgcgcccctggaacgcccggccgcaggcgaggtcctccgcgcgtggaggccgccaggggagtggaaactgacagagtcgcggggaaggggcgagaagcgggttgggagtgagcgaaggcaagcgagagctgcgagtgagtgcggaaggagggccaggaggggtggcggctgggtggggagagagggtgcaagacgagcggcgcgtgtcgggagcctttgggctgcgggtgcgttacaggagagcaggcgggtaggagccttcgcgggggccgagctcggaaggcggacggctgtgcccgcccaggggatgcgcccgggccggccgcgaaggtgccttcttccgggggcccggacgaccctgacacggcacgcgcgcgcttcgcagcctcaaagactccggggcctcgtggtcactggcgcaggggatcggggcggggtgcccggagtgcggtgcctcgtggctgaagggcagctcttctccccgcagtgcagagcagagcgggcggaggaccccgggcgcgggcgcggacggcacgcggggcatgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggtctccggcctcgggagccggcgggggcgcgccggggagggcccagagacagagcccggggtccccggcgccgcctccgaggcgagcccaggggaccgcgcggggcggacgggcgggcggcggaggcatcaggtggcgtcgccggagccgcaggaggaggaaaggaggcctcggctctcagcgggaccgcgggggccgggagccgggtcctgggcgcgtggaggctgcagggaaaccgctgaaggcccggccgggcccggggaagggcggccaaaggcttgaggggttttgcgcgttcgtccgtgcgttctcgtttccacgcaagcctcccgcccttcctccgggctcccgtctgccgcctccgtccgtgggtccccctcgccctctccgtgcgtccgcgcctgtgccggcggctgttttcgtctctcaccgcgtctctgtttctcttttcgctgcttttctctctgagtctctctctctccatgtttttcctgaggtcagcctctcttctcgctcctgctagctctctgcgggtactcccacctctgtctttctctttgtgtgtctctgtctctctctttcccccatcgcagtggaactcagggcctctgtctagagctgtctcccttgccctttgcgcgagtgcacacacgtgtgtcgttgttacgattgttctcccctaaggcagtttacccagagaactacgtgtctgggcccagcccccacctgtgggcagagcaggggaaggggacttcctccgggaatttggtctcaatttgctctcagagtgcctcagctgtgctgccatcc

SEQ ID NO: 168 - DNA, 3780 bp, Artificial Sequence
  Synthetic
ttgtaggttgatttacggtggtggtggtttaaaattatatagaaattagagatttgatggtttatttgaaaatgcgaagtaaatgtatatatttagtattttagacgatcgaaagtttaacgggtgaaagtttagggggttaattttgcgattaaggatattttggaaaagtatgtaaaatttttggtttatttaagtattatttttattttgggtttttatttatttattttgagatggagttttgttttgtcgtttatttcggtttattgtaattttagtttttcgggtttaagcgattttcgtggttcgtttttttaagtagttggggttacgttcggttaatttttgtatttttagtagagatggggttttatcgtgttggttaggttggtttcgaatttttgattttaggcgatttatcgtttcggttttttaaagtgttgggattataggtttgagttatagcgtttggtttattttgggtttttatattttattggtaaattgtttttttttaggttgaggttaaaacgatattattttaaggtgaattgaagtttggaagtgattaagtatttggatttttagggagttttttttcgtttttattttttttatgttaagataattaaaatttcggggtcggggtattgtttgagttattttaatttattagtttaatagatgtaaaaggatgtaagtatgttagatacgtagagatagtatttgaattaagtttaatagtttaagggagttgggttattttttattttttttttagtgagtgcgatgttttttaaatatcgcggagaggtagggttttcggtgatggttttgtcgagggtgttttcgtaatttttatttttagttttttttggattattttttcgtttttcgttatacgttttgtaaagcgaagtttttttcgtattagattttcgttattatacgttttttatttttcgttttggttatatcgttgtagtttagtgattgattggttttttgaggttttggcgtaaaggcgagattcgtatttcgtatttcgtttttcgcgggaaacggttttagtgatagttttcgaagcggcgcgcgttcggttggaggtgcgtttttcgggcgcggcgcgcggagggtcgttaagggcgcgggaattttatttcggtcgcggtagtttttagtttttacgtcggttttgaggttcgttcgttcggtcggtttcggttttcggtttgtaaagtttttttttttagtttaattttcggtttttataggtttcggcgtttcgtttcgttttaggtttcgttttagagagttttataaagttttttttttttatttcgtatttttagttttatcgttcgattttgggatttatcgttgtttttagtttcgagtttagggcgaggtaagggttggagtcgggtaggaggaggggtgtgaggttgatattagagaggattcggagcgcgaattagaggttcgatttttagggtagcgtagggtatttcggtttcggagcgggaagggagcgcgtttcgttttggagtttcgatttttattttatttgcgttgagtcggaggcgttggtttgggttttaaggttcgtttttttggttttgttcgagttttttcgtttgtttttcgcgtttttgcgacggggtcgttataagttggacgggatgagttaatcggattgtcggggttttaggagtggttgaggcggggtcgtttaaggtatttatataagacggtataattgtttgcgagaaataggttcggttttgtggattttaattcgaggtttttttttttgtttttcgtttttaaggggtttaagtttacggcggttttcggcgcggtgtttattcgttggcgtaggaggaggaggagttttatatttgggtcgtttcgagttttgcgtgcggtggtttagtcggtttggcgcggtttttgttttttaggtttcgtagttgtcgtcgttttttttcgcgtttttttcgttttcgttttttcgggtttgtttcggggttcggcggacgttttgcgcgcggaatttttcgtattggggttgtagttttcgcgtttgcgttatttgtcgtttgtagagtttatttagtgcgcgttagttgggtagggataggggttttattcggggcgaagggtttggatgcgagtagagaaagcggagggtggaggaattcggggttgcgtttttggaacgttcggtcgtaggcgaggtttttcgcgcgtggaggtcgttaggggagtggaaattgatagagtcgcggggaaggggcgagaagcgggttgggagtgagcgaaggtaagcgagagttgcgagtgagtgcggaaggagggttaggaggggtggcggttgggtggggagagagggtgtaagacgagcggcgcgtgtcgggagtttttgggttgcgggtgcgttataggagagtaggcgggtaggagttttcgcgggggtcgagttcggaaggcggacggttgtgttcgtttaggggatgcgttcgggtcggtcgcgaaggtgttttttttcgggggttcggacgattttgatacggtacgcgcgcgtttcgtagttttaaagatttcggggtttcgtggttattggcgtaggggatcggggcggggtgttcggagtgcggtgtttcgtggttgaagggtagttttttttttcgtagtgtagagtagagcgggcggaggatttcgggcgcgggcgcggacggtacgcggggtatgaatttggagggcggcggtcgaggcggagagttcggtatgagcgcggtgagttgcggtaacgggaagtttcgttagtggttgatcgattagatcgatagcggtaagtatttcgggttggtgtgggagaacgaggagaagagtatttttcgtattttttggaagtacgcgggtaagtaggattataatcgcgaggaggacgtcgcgttttttaaggttttcggtttcgggagtcggcgggggcgcgtcggggagggtttagagatagagttcggggttttcggcgtcgttttcgaggcgagtttaggggatcgcgcggggcggacgggcgggcggcggaggtattaggtggcgtcgtcggagtcgtaggaggaggaaaggaggtttcggtttttagcgggatcgcgggggtcgggagtcgggttttgggcgcgtggaggttgtagggaaatcgttgaaggttcggtcgggttcggggaagggcggttaaaggtttgaggggttttgcgcgttcgttcgtgcgttttcgtttttacgtaagtttttcgtttttttttcgggttttcgtttgtcgttttcgttcgtgggtttttttcgtttttttcgtgcgttcgcgtttgtgtcggcggttgttttcgttttttatcgcgtttttgttttttttttcgttgtttttttttttgagtttttttttttttatgttttttttgaggttagttttttttttcgtttttgttagttttttgcgggtatttttatttttgtttttttttttgtgtgtttttgttttttttttttttttatcgtagtggaatttagggtttttgtttagagttgttttttttgttttttgcgcgagtgtatatacgtgtgtcgttgttacgattgtttttttttaaggtagtttatttagagaattacgtgtttgggtttagtttttatttgtgggtagagtaggggaaggggatttttttcgggaatttggttttaatttgtttttagagtgttttagttgtgttgttattt

SEQ ID NO: 169 - DNA, 3780 bp, Artificial Sequence
  Synthetic
ttacaaattaacctacgataataataatctaaaatcatataaaaaccaaaaatctaataatttacctaaaaatacgaaataaatacacatactcaacatctcaaacgatcgaaaactcaacgaataaaaactcaaaaaattaattttacgattaaaaacatcttaaaaaaatatataaaatccctaatccacttaaatattattcctattttaaacttttatttatttattttaaaataaaatcttactctatcgcccacctcgactcactacaacttcaacctcccgaattcaaacgattctcgtaactcgtcctctcaaataactaaaaccacgcccgactaatttttatatttttaataaaaataaaatttcaccgtattaaccaaactaatctcgaactcctaacctcaaacgatccaccgcctcgacctcccaaaatactaaaattacaaacttaaaccacaacgcctaacctattttaaacttttataccccactaataaactactttcctccaaattaaaattaaaacgacatcattttaaaataaactaaaatctaaaaataattaaacacttaaatccttaaaaaacctctccccgcccccatctctttcatactaaaataattaaaacttcgaaaccgaaacattatctaaatcacttcaattcaccaacctaataaatacaaaaaaatataaacatatcaaacacgcaaaaacaatatttaaatcaaacttaataactcaaaaaaactaaaccatttcctattttctttttaataaatacgatattctctaaacaccgcgaaaaaacaaaattcccgataataaccttaccgaaaatactcccgcaacctccacctccaattctctttaaaccattcctccgtcttccgttacacgctctacaaaacgaaatccccttcgcaccaaattcccgctactacacgccccccatttcccgccctaaccacatcgctacaatttaataattaactaacctcctaaaatcctaacgcaaaaacgaaattcgcatttcgcacctcgcccttcgcgaaaaacgaccccaataacaatccccgaaacgacgcgcgcccgactaaaaatacgctctccgaacgcgacgcgcgaaaaatcgccaaaaacgcgaaaaccccaccccgaccgcgacaacccccaaccttcacgccgaccctaaaactcgcccgcccgaccgaccccgactctcgacttacaaaatccctctccccaatccaacccccgacccccacaaacctcgacgccccgccccgccccaaaccccgccccaaaaaattctataaaattcctctttcccacctcgcactctcaatttcaccgctcgatcttaaaacccaccgctaccctcaactccgaatccaaaacgaaataaaaactaaaatcgaacaaaaaaaaaaatataaaactaataccaaaaaaaacccgaaacgcgaaccaaaaattcgacctccaaaacaacgcaaaataccccgacttcgaaacgaaaaaaaaacgcgccccgtcctaaaactccgactcccaccccatctacgctaaaccgaaaacgctaatttaaactccaaaacccgcctccttaactctacccgaacctccccgcctaccctccgcgctcctacgacgaaatcgccacaaactaaacgaaataaactaaccgaactatcgaaaccccaaaaataactaaaacgaaaccgtccaaaacacccacacaaaacgacacaactacctacgaaaaacaaacccgaccctataaaccccaatccgaaactccttcccctactcttcgttcctaaaaaacccaaactcacgacgacctccgacgcgatactcacccgctaacgcaaaaaaaaaaaaaactccacatttaaatcgctccgaaccttacgtacgataacctaaccgacctaacgcgatccctacctcccaaactccgcaactatcgtcgccctctcccgcgccctccccgcctccgctctcccgaacctactccgaaatccgacgaacgctctacgcgcgaaatcccccgtactaaaactacaacccccgcgtctacgccacttatcgtttacaaaacccacttaatacgcgctaactaaacaaaaataaaaatcctattcgaaacgaaaaatctaaatacgaacaaaaaaaacgaaaaataaaaaaacccgaaactacgcccctaaaacgcccgaccgcaaacgaaatcctccgcgcgtaaaaaccgccaaaaaaataaaaactaacaaaatcgcgaaaaaaaaacgaaaaacgaattaaaaataaacgaaaacaaacgaaaactacgaataaatacgaaaaaaaaaccaaaaaaaataacgactaaataaaaaaaaaaaatacaaaacgaacgacgcgtatcgaaaacctttaaactacgaatacgttacaaaaaaacaaacgaataaaaaccttcgcgaaaaccgaactcgaaaaacgaacgactatacccgcccaaaaaatacgcccgaaccgaccgcgaaaataccttcttccgaaaacccgaacgaccctaacacgacacgcgcgcgcttcgcaacctcaaaaactccgaaacctcgtaatcactaacgcaaaaaatcgaaacgaaatacccgaaatacgatacctcgtaactaaaaaacaactcttctccccgcaatacaaaacaaaacgaacgaaaaaccccgaacgcgaacgcgaacgacacgcgaaacataaacctaaaaaacgacgaccgaaacgaaaaattcgacataaacgcgataaactacgacaacgaaaaactccgccaataactaatcgaccaaatcgacaacgacaaataccccgaactaatataaaaaaacgaaaaaaaaaacatcttccgcatcccctaaaaacacgcgaacaaacaaaactacaaccgcgaaaaaaacgccgcgctcttcaaaatctccgacctcgaaaaccgacgaaaacgcgccgaaaaaaacccaaaaacaaaacccgaaatccccgacgccgcctccgaaacgaacccaaaaaaccgcgcgaaacgaacgaacgaacgacgaaaacatcaaataacgtcgccgaaaccgcaaaaaaaaaaaaaaaaacctcgactctcaacgaaaccgcgaaaaccgaaaaccgaatcctaaacgcgtaaaaactacaaaaaaaccgctaaaaacccgaccgaacccgaaaaaaaacgaccaaaaacttaaaaaattttacgcgttcgtccgtacgttctcgtttccacgcaaacctcccgcccttcctccgaactcccgtctaccgcctccgtccgtaaatccccctcgccctctccgtacgtccgcgcctataccgacgactattttcgtctctcaccgcgtctctatttctcttttcgctacttttctctctaaatctctctctctccatatttttcctaaaatcaacctctcttctcgctcctactaactctctacgaatactcccacctctatctttctctttatatatctctatctctctctttcccccatcgcaataaaactcaaaacctctatctaaaactatctcccttaccctttacgcgaatacacacacgtatatcgttattacgattattctcccctaaaacaatttacccaaaaaactacgtatctaaacccaacccccacctataaacaaaacaaaaaaaaaaaacttcctccgaaaatttaatctcaatttactctcaaaatacctcaactatactaccatcc

SEQ ID NO: 170 - DNA, 23 bp, Artificial Sequence
  Synthetic
aaaaaaaaaaaaactccacattt

SEQ ID NO: 171 - DNA, 17 bp, Artificial Sequence
  Synthetic
  misc_feature (7)..(7): n is a, c, g, or t
tagttgnggagtttggg

SEQ ID NO: 172 - DNA, 23 bp, Artificial Sequence
  Synthetic
atcgtacgtaaggttcggagcga

(19) United States
(12) Patent Application Publication
(10) Pub. No.: US 2023/0193395 A1
(43) Pub. Date: Jun. 22, 2023

(54) METHODS AND KITS FOR SCREENING COLORECTAL NEOPLASM

(71) Applicant: SINGLERA HEALTH TECHNOLOGIES (SHANGHAI) LTD., Shanghai (CN)

(72) Inventors: Rui LIU, Shanghai (CN); Hui WANG, Shanghai (CN)

(73) Assignee: SINGLERA HEALTH TECHNOLOGIES (SHANGHAI) LTD., Shanghai (CN)

(21) Appl. No.: 17/423,461

(22) PCT Filed: Mar. 1, 2021
(86) PCT No.: PCT/CN2021/078445

§ 371 Date: Jul. 15, 2021

(30) Foreign Application Priority Data
Mar. 20, 2020 (WO) PCT/CN2020/080469
Jan. 29, 2021 (WO) PCT/CN2021/074409

(51) Int. Cl. C12Q 1/6886

(52) U.S. Cl. CPC C12Q 1/6886; C12Q 2600/154; C12Q 2600/118

(57) ABSTRACT

The present disclosure relates to a method of diagnosing colorectal neoplasm, screening for the onset or risk to the onset of colorectal neoplasm or assessing the development or prognosis of colorectal neoplasm in a subject, a method of monitoring treatment response in a subject who is receiving treatment of colorectal neoplasm, and a kit for using in the methods.

### FIELD OF THE INVENTION

The present disclosure generally relates to the biomedical field. In particular, the present disclosure relates to a method of diagnosing colorectal neoplasm, screening for the onset or risk to the onset of colorectal neoplasm or assessing the development or prognosis of colorectal neoplasm in a subject, a method of monitoring treatment response in a subject who is receiving treatment of colorectal neoplasm, and a kit for using in the methods.

### REFERENCE TO A SEQUENCE LISTING

This patent or application file contains a Sequence Listing submitted in computer readable ASCII text format (file name: 5354-2000230_SeqList_ST25.txt, date recorded: Sep. 1, 2022, and size: 431,721 bytes). The content of the Sequence Listing file is incorporated herein by reference in its entirety.

### BACKGROUND OF THE INVENTION

Early detection of colorectal neoplasm in the pre-cancerous advanced adenoma stage or early cancerous stage has been shown to significantly decrease patient mortality. Current colorectal neoplasm screening through colonoscopy or molecular tests on stool/blood samples is either invasive or has very few markers, limiting patient compliance to cancer screening and detection sensitivity.

Therefore, there is a growing need for developing a method and/or a kit that can efficiently read out epigenetics information from limited amount of cell-free DNA from a biological sample and can be easily deployed and robustly implemented in clinical laboratories.

### SUMMARY OF THE INVENTION

In one aspect, the present disclosure provides a method of diagnosing colorectal neoplasm, screening for the onset or risk to the onset of colorectal neoplasm or assessing the development or prognosis of colorectal neoplasm in a subject, said method comprising the following steps:
(I). treating a DNA obtained from a biological sample with a reagent capable of distinguishing between an unmethylated site and a methylated site in the DNA, thereby obtaining a treated DNA;
(II). quantifying individual methylation level of a set of target markers within the treated DNA of step (I), wherein the target markers are selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(III). comparing the methylation level of at least one target marker of the set of target markers quantified at step (II) respectively with a corresponding reference level, wherein an identical or higher methylation level of one or more of the target markers relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In another aspect, the present disclosure provides a method of diagnosing colorectal neoplasm, screening for the onset or risk to the onset of colorectal neoplasm or assessing the development or prognosis of colorectal neoplasm in a subject, said method comprising the following steps:
(I). treating a DNA obtained from a biological sample with a reagent capable of distinguishing between an unmethylated site and a methylated site in the DNA, thereby obtaining a treated DNA;
(II). quantifying individual methylation level of a set of target markers within the treated DNA of step (I), wherein at least two target markers are selected from the group consisting of Septin9, BCAT1, IKZF1, BCAN, PKNOX2, VAV3, NDRG4 and IRF4, and at least two target markers are selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP.
(III). comparing the methylation level of at least one target marker of the set of target markers quantified at step (II) respectively with a corresponding reference level, wherein an identical or higher methylation level of one or more of the target markers relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In some embodiments, the set of target markers of the present disclosure comprises 1, 2, 3, 4, 5, 6, 7, 8, 9, 10, 11, 12, 13, 14, 15, 16, 17, 18, 19, 20, 21, 22, 23, 24, 25, 26, 27, 28 or more target markers.

In some embodiments, the step (II) of the present disclosure comprises:
(i) pre-amplifying at least a portion of at least one target marker of a set of target markers within the treated DNA obtained from step (I) with a pre-amplification primer pool, and the set of target markers are selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(ii) quantifying individual methylation level of the set of target markers within achieved DNA from the said sub-step (i).

In some embodiments, the method of the present disclosure further comprises obtaining DNA from a biological sample from a subject before the step (I).

In another aspect, the present disclosure provides a method of diagnosing colorectal neoplasm, screening for the onset or risk to the onset of colorectal neoplasm or assessing the development or prognosis of colorectal neoplasm in a subject, said method comprises the following steps:
(a) obtaining a biological sample containing DNA from the subject;
(b) treating the DNA in the biological sample obtained from step (a) with a reagent capable of distinguishing between an unmethylated site and a methylated site in the DNA, thereby obtaining a treated DNA;
(c) pre-amplifying at least a portion of at least one target marker within the treated DNA obtained from step (b) with a pre-amplification primer pool, wherein at least a portion of at least one (e.g. each) of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d) if step (c) is present, then quantifying individually methylation level of the at least one (e.g. each) target marker based on achieved DNA from step (c); if step (c) is absent, then quantifying individually methylation level of at least one (e.g. each) target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(e) comparing the methylation level of at least one (e.g. each) target marker from step (d) respectively with a corresponding reference level, wherein an identical or higher methylation level of one or more of the target marker(s) relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In some embodiments, the at least one target marker in step (c) or step (d) of the method above comprises multiple target markers, wherein the multiple target markers comprise at least two markers selected from the group consisting of Septin9, BCAT1, and IKZF1.

In another aspect, the present disclosure provides a method of monitoring treatment response in a subject who is receiving treatment of colorectal neoplasm, comprising the following steps:
(a) obtaining a biological sample containing DNA from the subject;
(b) treating the DNA in the biological sample obtained from step (a) with a reagent capable of distinguishing between an unmethylated site and a methylated site in the DNA, thereby obtaining a treated DNA;
(c) pre-amplifying at least a portion of at least one target marker within the treated DNA obtained from step (b) with a pre-amplification primer pool, wherein at least a portion of at least one (e.g. each) of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d) if step (c) is present, then quantifying individually methylation level of the at least one (e.g. each) target marker based on achieved DNA from step (c); if step (c) is absent, then quantifying individually methylation level of at least one (e.g. each) target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(e) comparing the methylation level of at least one (e.g. each) target marker from step (d) respectively with a corresponding methylation level of one or more of the target marker(s) obtained from the same subject prior to the treatment which is quantified by repeating step (a), step (b), optionally step (c), and step (d) with respect to a biological sample containing DNA obtained from the subject prior to the treatment, wherein a lower methylation level of one or more of the target marker(s) relative to its corresponding methylation level prior to the treatment indicates that the subject is responsive to the treatment.

In some embodiments, the at least one target marker in step (c) or step (d) of the method above comprises multiple target markers, wherein the multiple target markers comprise at least two markers selected from the group consisting of Septin9, BCAT1, and IKZF1.

In some embodiments, the multiple target markers further comprise one or more additional markers selected from the group consisting of BCAN, PKNOX2, VAV3, NDRG4, and IRF4. In some embodiments, the multiple target markers further comprise one or more additional markers selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP.

In some embodiments, the respective target marker comprises or is: a) the respective region defined by Hg19 coordinates as set forth below:

| Target Marker | Hg19 Coordinate |
|---|---|
| NDRG4 | chr16: 58496750-58547532 |
| BCAT1 | chr12: 24964295-25102393 |
| IKZF1 | chr7: 50343720-50472799 |
| Septin9 | chr17: 75276651-75496678 |
| SDC2 | chr8: 97505579-97624000 |
| VAV3 | chr1: 108113782-108507766 |
| IRF4 | chr6: 391739-411447 |
| TMEFF2 | chr2: 192813769-193060435 |
| SALL1 | chr16: 51169886-51185278 |
| BCAN | chr1: 156611182-156629324 |
| POU4F2 | chr4: 147560045-147563626 |
| PKNOX2 | chr11: 125034583-125303285 |
| ASCL4 | chr12: 108168162-108170421 |
| KCNA6 | chr12: 4918342-4960277 |
| SOX1 | chr13: 112721913-112726020 |
| HS3ST2 | chr16: 22825498-22927659 |
| FGF12 | chr3: 191857184-192485553 |
| KCTD8 | chr4: 44175926-44450824 |
| HMX1 | chr4: 8847802-8873543 |
| MARCH11 | chr5: 16067248-16180871 |
| CRHBP | chr5: 76248538-76276983 |
| NKX2-6 | chr8: 23559964-23564111 |
| SLC24A2 | chr9: 19507450-19786926 |
| INTERGENIC REGION 1 | chr6: 19679885-19693988 |
| INTERGENIC REGION 2 | chr10: 130082033-130087148 |
| INTERGENIC REGION 3 | chr10: 133107880-133113966 |
| INTERGENIC REGION 4 | chr7: 152620588-152624685 |
| INTERGENIC REGION 5 | chr8: 70945014-70949177 |

, and 5 kb upstream of the respective start site and 5 kb downstream of the respective end site of each region described above, or b) a bisulfite converted counterpart of a), or c) a MSRE treated counterpart of a).

In some embodiments, the DNA in the biological sample obtained from step (a) comprises genomic DNA or cell-free DNA. In some embodiments, the cell-free DNA comprises circulating tumor DNA. In some embodiments, the target marker in the cell-free DNA is present in the biological sample in an amount no more than 1 ng, 0.8 ng, 0.6 ng, 0.4 ng, 0.2 ng, 0.1 ng, 0.08 ng or no more than 0.04 ng. In some embodiments, the target marker in the cell-free DNA is present in the biological sample at a concentration that is below a level of sensitivity of a detection assay for the target marker.

In some embodiments, the achieved DNA from sub-step (i) or step (c) is diluted with a diluent prior to sub-step (ii) or step (d).

In some embodiments, the biological sample is selected from the group consisting of a tissue section, biopsy, a paraffin-embedded tissue, a body fluid, colonic effluent, a surgical resection sample, an isolated blood cell, a cell isolated from blood, and any combination thereof. In some embodiments, the body fluid is selected from the group consisting of whole blood, blood serum, blood plasma, urine, mucus, saliva, peritoneal fluid, pleural fluid, chest fluid, synovial fluid, cerebrospinal fluid, thoracentesis fluid, abdominal fluid, and any combination thereof. In some embodiments, the biological sample is obtained from blood plasma of the subject. In some embodiments, the colonic effluent is selected from the group consisting of a stool sample and an enema wash sample.

In some embodiments, the reagent capable of distinguishing between an unmethylated site and a methylated site in the DNA selectively modifies at unmethylated cytosine residue(s) at the CpG site(s) to produce modified residue(s) but does not significantly modify methylated cytosine residue(s). In some embodiments, the reagent capable of distinguishing between an unmethylated site and a methylated site in the DNA comprises a bisulfite reagent. In some embodiments, the bisulfite reagent is selected from the group consisting of ammonium bisulfite, sodium bisulfite, potassium bisulfite, calcium bisulfite, magnesium bisulfite, aluminum bisulfite, hydrogen sulfite and any combination thereof.

In some embodiments, the reagent capable of distinguishing between an unmethylated site and a methylated site in the DNA selectively cleaves at a residue when it is unmethylated but does not cleave at the residue when it is methylated, or selectively cleaves at the residue when it is methylated but does not cleave at the residue when it is unmethylated. In some embodiments, the reagent capable of distinguishing between an unmethylated site and a methylated site in the DNA is a methylation sensitive restriction enzyme (MSRE). In some embodiments, the MSRE is selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI and any combination thereof.

In some embodiments, the pre-amplification primer pool comprises at least one methylation-specific primer pair. In some embodiments, the at least one methylation-specific primer pair comprises a forward primer and a reverse primer each comprising an oligonucleotide sequence that hybridizes under stringent conditions, moderately stringent conditions, or highly stringent conditions to at least 9 consecutive nucleotides of one of the target marker(s), wherein the at least 9 consecutive nucleotides of one of the target marker(s) comprise at least one CpG site.

In some embodiments, the pre-amplification primer pool further comprises a control primer pair for amplifying a control marker. In some embodiments, the control marker is selected from the group consisting of ACTB, GAPDH, tubulin, ALDOA, PGKT, LDHA, RPS27A, RPL19, RPL11, ARHGDIA, RPL32, Clorf43, CHMP2A, EMC7, GPI, PSMB2, PSMB4, RAB7A, REEP5, SNRPD3, VCP, and VPS29.

In some embodiments, the at least one methylation-specific primer pair comprises one or more pairs of nucleotide sequences selected from the group consisting of SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, and 170/171, as shown in Table 2 below.

In some embodiments, in step (c), the at least one target marker is amplified in the presence of one or more blocker oligonucleotides.

In some embodiments, the quantifying is conducted by polymerase chain reaction (PCR) (e.g. real-time PCR, digital PCR), nucleic acid sequencing, mass-based separation (e.g. electrophoresis, mass spectrometry), or target capture (e.g. hybridization, microarray). In some embodiments, the quantifying is conducted by the real-time PCR, optionally the real-time PCR is multiplexed real-time PCR.

In some embodiments, if step (c) is present, then the quantifying of step (d) comprises amplifying the achieved DNA from step (c) using quantification primer pair(s) and a DNA polymerase, wherein the at least a portion of the achieved DNA is amplified. In some embodiments, if step (c) is absent, then the quantifying of step (d) comprises amplifying the at least one target marker within the treated DNA obtained from step (b) using quantification primer pair(s) and a DNA polymerase.

In some embodiments, if step (c) is present, then the quantification primer pair(s) used in step (d) is (are) capable of hybridizing to at least 9 consecutive nucleotides of the achieved DNA from step (c) under stringent conditions, moderately stringent conditions, or highly stringent conditions. In some embodiments, if step (c) is absent, then the quantification primer pair(s) used in step (d) is (are) capable of hybridizing to at least 9 consecutive nucleotides of the at least one target marker within the treated DNA obtained from step (b) under stringent conditions, moderately stringent conditions, or highly stringent conditions.

In some embodiments, if step (c) is present, then at least one of the quantification primer pair(s) used in step (d) is (are) identical to at least one of the methylation-specific primer pair(s) in the pre-amplification primer pool of step (c).

In some embodiments, if step (c) is present, then the quantification primer pair(s) used in step (d) is (are) designed to amplify at least a portion within the achieved DNA from step (c). In some embodiments, if step (c) is absent, then the quantification primer pair(s) used in step (d) is (are) designed to amplify at least a portion within the at least one target marker within the treated DNA obtained from step (b).

In some embodiments, the step (d) is conducted in the presence of a detection agent. In some embodiments, the detection agent is selected from the group consisting of a fluorescent probe, an intercalating dye, a chromophore-labeled probe, a radioisotope-labeled probe, and a biotin-labeled probe. In some embodiments, the fluorescent probe comprises a nucleotide sequence selected from the group consisting of SEQ ID NOs: 57-85, 172. In some embodiments, the fluorescent probe is labeled with a fluorescent dye (e.g. FAM, HEX/VIC, TAMRA, Texas Red, or Cy5) at its 5′ end, and labeled with a quencher (e.g. BHQ1, BHQ2, BHQ3, DABCYL or TAMRA) at its 3′ end.

In some embodiments, step (e) comprises comparing Ct value(s) of the target marker(s) of step (d) with a reference Ct value, wherein an identical or lower Ct value of at least one target marker relative to its corresponding reference Ct value indicates that the subject has colorectal neoplasm, is at the onset or at a risk to the onset of colorectal neoplasm, or develops or with an increased probability of developing colorectal neoplasm, or has poor prognosis or at a risk to poor prognosis of colorectal neoplasm; or a higher Ct value of at least one target marker relative to its corresponding Ct value prior to the treatment indicates that the subject who is receiving the treatment of colorectal neoplasm is responsive to the treatment.

In some embodiments, the pre-amplification comprises from 5 to 30 cycles of reaction, wherein each cycle comprises reaction at 85˜99° C. for 5s to 5 mins before reaction at 40˜80° C. for 5s-5 mins.

In some embodiments, if step (c) is present, then the quantifying of step (d) comprises determining the methylation level based on presence or level of a plurality of CpG dinucleotides, TpG dinucleotides, or CpA dinucleotides in the achieved DNA from step (c). In some embodiments, if step (c) is absent, then the quantifying of step (d) comprises determining the methylation level based on presence or level of a plurality of CpG dinucleotides, TpG dinucleotides, or CpA dinucleotides in the at least one target marker within the treated DNA obtained from step (b). In some embodiments, if step (c) is present, then the quantifying of step (d) comprises determining methylation level of cytosine residue(s) based on presence or level of one or more CpG dinucleotides in the achieved DNA from step (c). In some embodiments, if step (c) is absent, then the quantifying of step (d) comprises determining methylation level of cytosine residue(s) based on presence or level of one or more CpG dinucleotides in the at least one target marker within the treated DNA obtained from step (b). In some embodiments, if step (c) is present, then the quantifying of step (d) is performed by partitioning the achieved DNA from step (c) into a plurality of fractions. In some embodiments, if step (c) is absent, then the quantifying of step (d) is performed by partitioning the at least one target marker within the treated DNA obtained from step (b) into a plurality of fractions.

In some embodiments, the reference levels of step (e) are determined based on the clinical samples obtained from a group of individuals having or at the risk of having colorectal neoplasm and a group of individuals without or are free of the risk of having colorectal neoplasm.

In some embodiments, the colorectal neoplasm is a colorectal cancer, a large colorectal adenoma, and/or a sessile serrated polyp. In some embodiments, the colorectal neoplasm is pre-cancerous. In some embodiments, the subject is a human.

In another aspect, the present disclosure provides a kit for diagnosing colorectal neoplasm, screening for the onset or risk to the onset of colorectal neoplasm or assessing the development or prognosis of colorectal neoplasm, comprising:
(a) a first reagent for treating a DNA, wherein the first reagent is capable of distinguishing between an unmethylated site and a methylated site in the DNA;
(b) optionally a first primer pool comprising at least one primer pair for pre-amplifying at least one target sequence in at least one target marker selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5, wherein the at least one primer pair is capable of hybridizing under stringent conditions, moderately stringent conditions, or highly stringent conditions to at least 9 consecutive nucleotides of the at least one target sequence treated by the first reagent; and wherein the target sequence comprises at least one CpG site; and
(c) a second reagent, wherein if the first primer pool is present, then the second reagent is for quantifying methylation level of the at least one (e.g. each) target marker pre-amplified by the first primer pool; if the first primer pool is absent, then the second reagent is for quantifying methylation level of at least one (e.g. each) target marker within the DNA treated by the first reagent, wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5.

In some embodiments, the at least one target marker comprises multiple target markers, wherein the multiple target markers comprise at least two markers selected from the group consisting of Septin9, BCAT1, and IKZF1.

In some embodiments, if the first primer pool is present, then the second reagent comprises a second primer pool comprising multiple quantification primer pairs capable of hybridizing under stringent conditions, moderately stringent conditions, or highly stringent conditions to at least 9 consecutive nucleotides of the at least one target sequence pre-amplified by the first primer pool. In some embodiments, if the first primer pool is absent, then the second reagent comprises a third primer pool comprising multiple quantification primer pairs capable of hybridizing under stringent conditions, moderately stringent conditions, or highly stringent conditions to at least 9 consecutive nucleotides of the at least one target sequence of the at least one target marker within the DNA treated by the first reagent.

In some embodiments, at least one of the quantification primer pairs in the second primer pool is identical to at least one of the primer pairs in the first primer pool. In some embodiments, if the first primer pool is present, then quantification primer pairs of the second primer pool are designed to amplify at least a portion within the at least one target sequence pre-amplified by the first primer pool. In some embodiments, if the first primer pool is absent, then quantification primer pairs of the third primer pool are designed to amplify at least a portion within the at least one target sequence of the at least one target marker within the DNA treated by the first reagent.

In some embodiments, the first, second, or third primer pool comprises at least one methylation-specific primer pair.

In some embodiments, the first primer pool and the second primer pool are packaged in a single container or in separate containers. In some embodiments, the kit further comprises one or more blocker oligonucleotides.

In some embodiments, the kit further comprises a detection agent. In some embodiments, the detection agent is selected from the group consisting of a fluorescent probe, an intercalating dye, a chromophore-labeled probe, a radioisotope-labeled probe, and a biotin-labeled probe. In some embodiments, the fluorescent probe comprises an oligonucleotide sequence selected from the group consisting of SEQ ID NOs: 57-85, 172. In some embodiments, the fluorescent probe is labeled with a fluorescent dye (e.g. FAM, HEX/VIC, TAMRA, Texas Red, or Cy5) at its 5′ end, and labeled with a quencher (e.g. BHQ1, BHQ2, BHQ3, DABCYL, TAMRA or lowa Black Dark Quenchers) at its 3′ end.

In some embodiments, the kit further comprises a DNA polymerase and/or a container suitable for containing the biological sample from the subject. In some embodiments, the kit further comprises an instruction for use and/or interpretation of the kit results.

In some embodiments, the first reagent comprises a bisulfite reagent or methylation sensitive restriction enzyme (MSRE). In some embodiments, the bisulfite reagent is selected from the group consisting of ammonium bisulfite, sodium bisulfite, potassium bisulfite, calcium bisulfite, magnesium bisulfite, aluminum bisulfite, hydrogen sulfite and any combination thereof. In some embodiments, the MSRE is selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI and any combination thereof.

In some embodiments, if the first primer pool is present, then the first primer pool comprises multiple primer pairs for pre-amplifying at least one target sequence in multiple target markers, wherein the multiple target markers comprise at least two markers selected from the group consisting of Septin9, BCAT1, and IKZF1, and further comprise one or more additional markers selected from the group consisting of BCAN, PKNOX2, VAV3, NDRG4, and IRF4. In some embodiments, if the first primer pool is absent, then the third primer pool comprises multiple primer pairs for amplifying at least one target sequence in multiple target markers, wherein the multiple target markers comprise at least two markers selected from the group consisting of Septin9, BCAT1, and IKZF1, and further comprise one or more additional markers selected from the group consisting of BCAN, PKNOX2, VAV3, NDRG4, and IRF4. In some embodiments, the multiple target markers further comprise one or more additional markers selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP.

In some embodiments, the respective target marker comprises or is: a) the respective region defined by Hg19 coordinates as set forth below:

| Target Marker | Hg19 Coordinate |
|---|---|
| NDRG4 | chr16: 58496750-58547532 |
| BCAT1 | chr12: 24964295-25102393 |
| IKZF1 | chr7: 50343720-50472799 |
| Septin9 | chr17: 75276651-75496678 |
| SDC2 | chr8: 97505579-97624000 |
| VAV3 | chr1: 108113782-108507766 |
| IRF4 | chr6: 391739-411447 |
| TMEFF2 | chr2: 192813769-193060435 |
| SALL1 | chr16: 51169886-51185278 |
| BCAN | chr1: 156611182-156629324 |
| POU4F2 | chr4: 147560045-147563626 |
| PKNOX2 | chr11: 125034583-125303285 |
| ASCL4 | chr12: 108168162-108170421 |
| KCNA6 | chr12: 4918342-4960277 |
| SOX1 | chr13: 112721913-112726020 |
| HS3ST2 | chr16: 22825498-22927659 |
| FGF12 | chr3: 191857184-192485553 |
| KCTD8 | chr4: 44175926-44450824 |
| HMX1 | chr4: 8847802-8873543 |
| MARCH11 | chr5: 16067248-16180871 |
| CRHBP | chr5: 76248538-76276983 |
| NKX2-6 | chr8: 23559964-23564111 |
| SLC24A2 | chr9: 19507450-19786926 |
| INTERGENIC REGION 1 | chr6: 19679885-19693988 |
| INTERGENIC REGION 2 | chr10: 130082033-130087148 |
| INTERGENIC REGION 3 | chr10: 133107880-133113966 |
| INTERGENIC REGION 4 | chr7: 152620588-152624685 |
| INTERGENIC REGION 5 | chr8: 70945014-70949177, |

and 5 kb upstream of the respective start site and 5 kb downstream of the respective end site of each region described above, or b) a bisulfite converted counterpart of a), or c) a MSRE treated counterpart of a).

In some embodiments, if the first primer pool is present, then the first primer pool comprises at least one primer pair comprising or consisting of at least one pair of nucleotide sequences selected from the group consisting of SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, and 170/171 as shown in Table 2 below, and optionally wherein the second primer pool comprises at least one primer pair that is identical to at least one of the primer pairs in the first primer pool. In some embodiments, if the first primer pool is absent, then the third primer pool comprises at least one primer pair comprising or consisting of at least one pair of nucleotide sequences selected from the group consisting of SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, and 170/171 as shown in Table 2 below.

In some embodiments, the first primer pool, the second primer pool, or optionally the third primer pool further comprises a primer pair for amplifying a control marker. In some embodiments, the control marker is selected from the group consisting of ACTB, GAPDH, tubulin, ALDOA, PGK1, LDHA, RPS27A, RPL19, RPL11, ARHGDIA, RPL32, Clorf43, CHMP2A, EMC7, GPI, PSMB2, PSMB4, RAB7A, REEP5, SNRPD3, VCP, and VPS29.

In some embodiments, the kit further comprises a plurality of containers, each for receiving a fraction of the second primer pool.

In another aspect, the present disclosure provides use of the kit of the present disclosure in the manufacture of a diagnostic kit for diagnosing colorectal neoplasm, screening for the onset or risk to the onset of colorectal neoplasm, or assessing the development or prognosis of colorectal neoplasm in the subject, or monitoring treatment response in a subject who is receiving treatment of colorectal neoplasm.

In another aspect, the present disclosure provides use of a reagent for quantifying methylation level of a target marker in the manufacture of a kit for using in a method of diagnosing colorectal neoplasm, screening for the onset or risk to the onset of colorectal neoplasm, or assessing the development or prognosis of colorectal neoplasm in a subject, wherein said method comprising the following steps:
(a) obtaining a biological sample containing DNA from the subject;
(b) treating the DNA in the biological sample obtained from step (a) with a reagent capable of distinguishing between unmethylated and methylated CpG site(s) in the DNA, thereby obtaining a treated DNA;
(c) pre-amplifying at least a portion of at least one target marker within the treated DNA obtained from step (b) with a pre-amplification primer pool, wherein at least a portion of at least one (e.g. each) of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d) if step (c) is present, then quantifying individually methylation level of the at least one (e.g. each) target marker based on achieved DNA from step (c); if step (c) is absent, then quantifying individually methylation level of at least one target (e.g. each) marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(e) comparing the methylation level of at least one (e.g. each) target marker from step (d) respectively with a corresponding reference level, wherein an identical or higher methylation level of at least one target marker relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In another aspect, the present disclosure provides use of a reagent for quantifying methylation level of a target marker in the manufacture of a kit for using in a method of monitoring treatment response in a subject who is receiving treatment of colorectal neoplasm, wherein said method comprising the following steps:
(a) obtaining a biological sample containing DNA from the subject;
(b) treating the DNA in the biological sample obtained from step (a) with a reagent capable of distinguishing between unmethylated and methylated CpG site(s) in the DNA, thereby obtaining a treated DNA;
(c) pre-amplifying at least a portion of at least one target marker within the treated DNA obtained from step (b) with a pre-amplification primer pool, wherein at least a portion of at least one (e.g. each) of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d) if step (c) is present, then quantifying individually methylation level of the at least one (e.g. each) target marker based on achieved DNA from step (c); if step (c) is absent, then quantifying individually methylation level of at least one (e.g. each) target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(e) comparing the methylation level of at least one (e.g. each) target marker from step (d) respectively with a corresponding methylation level of one or more of the target marker(s) obtained from the same subject prior to the treatment which is quantified by repeating step (a), step (b), optionally step (c), and step (d) with respect to a biological sample containing DNA obtained from the subject prior to the treatment, wherein a lower methylation level of one or more of the target marker(s) relative to its corresponding methylation level prior to the treatment indicates that the subject is responsive to the treatment.

### BRIEF DESCRIPTION OF THE FIGURES

FIG. 1 illustrates the verification of methylation-specific primers for target marker PKNOX2 (FIG. 1A) and control marker ACTB (FIG. 1B). The Y-axis shows A Rn value, which is determined by subtracting the baseline fluorescence intensity from the fluorescence intensity at the indicated cycle. The X-axis shows the number of cycles. As shown in FIG. 1A, the Ct values decreased as the percentage of the converted methylation DNA increased in the mixed DNA composition, which indicated that the primers used for pre-amplifying PKNOX2 were methylation-specific. As shown in FIG. 1B, the curves for each DNA composition overlapped, which indicated that the Ct values remained the same despite of increase in the percentage of the converted methylation DNA, and this is consistent with the fact that the primers used for pre-amplifying control marker ACTB were methylation-non-specific.

FIG. 2 illustrates the methylation abundances of control marker ACTB, and target marker SALL1 and PKNOX2 in white blood cells (WBC, indicated by solid circle “●”), paracancerous tissues (para-tissue, indicated by solid box “▪”), advanced adenoma tissues (AA-tissue, indicated by solid positive triangle “▴”), and colorectal cancer tissues (CRC-tissue, indicated b solid reverse triangle “▾”), respectively. The Y-axis shows the Ct values, and the X-axis shows the names of the control marker and target markers. A higher Ct value indicates a lower methylation abundance of a marker. Therefore, it can be seen from FIG. 2 that the methylation abundances of the target markers in white blood cells were significantly lower than in tissue samples. In particular, the methylation abundances of the target markers were lower in paracancerous tissues than in advanced adenoma tissues and colorectal cancer tissues.

FIG. 3 illustrates the distributions of control marker ACTB and target markers SALL1 and BCAN in biological samples obtained from population with colorectal cancer (CRC plasma, indicated by solid circle “●”) and population with negative colonoscopy (healthy plasma, indicated by solid positive triangle “▴”), respectively. The Y-axis shows the Ct values, and the X-axis shows the names of the control marker and target markers. A lower Ct value indicates a higher methylation level of a marker. Therefore, it can be seen from FIG. 3 that the methylation level of each target marker in population with colorectal cancer was significantly higher than that in population with negative colonoscopy.

FIG. 4 illustrates the AUC values of all tested 13 target markers. The Y-axis shows the number of occurrence in the same range of AUC value, and the X-axis shows the AUC value. AUC value is between 0 and 1, and a larger AUC value reprsents a better classification power. As shown in the figure, all tested markers (i.e. NDRG4, Septin9, BCAT1, IKZF1, BCAN, VAV3, POU4F2, SALL1, PKNOX2, SDC2, ASCL4, TMEFF2 and INTERGENIC REGION 1) had classification power to separate CRC from controls with an AUC ranging from 0.8 to 0.9.

FIG. 5 illustrates the ROC curve of the combination of markers SALL1, BCAT1 and Septin9. The Y-axis shows the true positive rate (i.e. sensitivity), and the X-axis shows the false positive rate (i.e. 1-specificity). The solid line indicates the ROC curve, and the dotted line indicates the 45 degree diagonal line. Points above the diagonal line represent good classification results (i.e. better than random), and points below the line represent poor results (i.e. worse than random). Therefore, the combination of target markers SALL1, BCAT1 and Septin9 has high sensitivity and high specificity in classifying colorectal neoplasm.

FIG. 6 shows the nucleotide sequences of exemplary subregions of the target markers.

### DETAILED DESCRIPTION OF THE INVENTION

Although various aspects and embodiments of the present disclosure will be disclosed in the following, a person skilled in the art can make various equivalent changes and modifications without departing from the spirit and scope of the subject matter of the application. The various aspects and embodiments disclosed herein are given by way of illustration only, and are not intended to limit the present disclosure. The actual protection scope of the present application is defined by the claims. Unless defined otherwise, all technical and scientific terms used herein have the same meaning as commonly understood by a person having ordinary skills in the art to which this invention pertains. All references, patents, patent applications cited in the present disclosure are hereby incorporated by reference in their entireties.

It must be noted that, as used in the specification and the appended claims, the singular forms “a,” “an,” and “the” include plural forms of the same unless the context clearly dictates otherwise. Thus, for example, reference to “a reagent” includes a plurality of reagents.

Throughout the specification and the claims which follow, unless the context requires otherwise, the word “comprise”, “contain” or “include”, and variations such as “comprises”, “comprising”, “contains”, “containing”, “includes”, and “including” will be understood to imply the inclusion of a stated integer or step or group of integers or steps but not the exclusion of any other integer or step or group of integers or steps.

Cancer diagnostics has traditionally relied upon the detection of single markers (e.g., gene mutations). Unfortunately, cancer is a disease state in which single markers have typically failed to detect or differentiate many forms of the disease. In addition, the level of a single marker in a biological sample is usually very limited, which further reduces the diagnostic specificity and/or diagnostic sensitivity of cancers. Thus, assays that recognize only a single marker have been shown to be of limited predictive value.

One aspect of the present disclosure is to pre-amplify at least a portion of at least one target marker so that at least a portion of at least one of the target marker(s) is pre-amplified, prior to quantifying individually methylation level of the at least one (e.g. each) target marker based on the achieved DNA from the pre-amplification. Such a pre-amplification step is believed to increase the amount(s)/level(s) of the target marker(s), and is found to significantly increase the diagnostic specificity and/or diagnostic sensitivity of colorectal neoplasm. Another aspect of the present disclosure is to simultaneously quantify methylation levels of multiple target markers within the biological sample so as to increase the diagnostic specificity and/or diagnostic sensitivity of colorectal neoplasm. In certain embodiments, the multiple target markers are not pre-amplified before being quantified. In certain embodiments, the multiple target markers are pre-amplified before being quantified. In particular, the inventors of the present disclosure surprisingly found that the simultaneous quantification of methylation levels of multiple target markers within the biological sample, or the combination of a pre-amplification step and a quantification step significantly increase the diagnostic specificity and/or diagnostic sensitivity of colorectal neoplasm, which makes it possible for early detection of colorectal neoplasm, for example in the pre-cancerous adenoma stage or early cancerous stage. As would be understood by the person of skill in the art, in the context of diagnostic “sensitivity” defines the proportion of positive results which are correctly identified, that is, the percentage of subjects correctly identified as having the disease at issue. “Specificity”, however, defines the proportion of negative results which are correctly identified, that is, the percentage of subjects correctly identified as not having the disease at issue.

### Methods

In one aspect, the present disclosure provides a method of diagnosing colorectal neoplasm, screening for the onset or risk to the onset of colorectal neoplasm or assessing the development or prognosis of colorectal neoplasm in a subject, said method comprises the following steps:
(I). treating a DNA obtained from a biological sample with a reagent capable of distinguishing between an unmethylated site and a methylated site in the DNA, thereby obtaining a treated DNA;
(II). quantifying individual methylation level of a set of target markers within the treated DNA of step (I), wherein the target markers are selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(III). comparing the methylation level of at least one target marker of the set of target markers quantified at step (II) respectively with a corresponding reference level, wherein an identical or higher methylation level of one or more of the target markers relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In another aspect, the present disclosure provides a method of diagnosing colorectal neoplasm, screening for the onset or risk to the onset of colorectal neoplasm or assessing the development or prognosis of colorectal neoplasm in a subject, said method comprises the following steps:
(I). treating a DNA obtained from a biological sample with a reagent capable of distinguishing between an unmethylated site and a methylated site in the DNA, thereby obtaining a treated DNA;
(II). quantifying individual methylation level of a set of target markers within the treated DNA of step (I), wherein at least two target markers are selected from the group consisting of Septin9, BCAT1, IKZF1, BCAN, PKNOX2, VAV3, NDRG4 and IRF4, and at least two target markers are selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP; and
(III). comparing the methylation level of at least one target marker of the set of target markers quantified at step (II) respectively with a corresponding reference level, wherein an identical or higher methylation level of one or more of the target markers relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In another aspect, the present disclosure provides a method of monitoring treatment response in a subject who is receiving treatment of colorectal neoplasm, comprising the following steps:
(I). treating a DNA obtained from a biological sample with a reagent capable of distinguishing between an unmethylated site and a methylated site in the DNA, thereby obtaining a treated DNA;
(II). quantifying individual methylation level of a set of target markers within the treated DNA of step (I), wherein the target markers are selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(III). comparing the methylation level of at least one target marker of the set of target markers quantified at step (II) respectively with a corresponding methylation level of one or more of the target marker(s) obtained from the same subject prior to the treatment which is quantified by repeating step (I) and step (II) with respect to a biological sample containing DNA obtained from the subject prior to the treatment, wherein a lower methylation level of one or more of the target marker(s) relative to its corresponding methylation level prior to the treatment indicates that the subject is responsive to the treatment.

In another aspect, the present disclosure provides a method of monitoring treatment response in a subject who is receiving treatment of colorectal neoplasm, comprising the following steps:
(I). treating a DNA obtained from a biological sample with a reagent capable of distinguishing between an unmethylated site and a methylated site in the DNA, thereby obtaining a treated DNA;
(II). quantifying individual methylation level of a set of target markers within the treated DNA of step (I), wherein at least two target markers are selected from the group consisting of Septin9, BCAT1, IKZF1, BCAN, PKNOX2, VAV3, NDRG4 and IRF4, and at least two target markers are selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP; and
(III). comparing the methylation level of at least one target marker of the set of target markers quantified at step (II) respectively with a corresponding methylation level of one or more of the target marker(s) obtained from the same subject prior to the treatment which is quantified by repeating step (I) and step (II) with respect to a biological sample containing DNA obtained from the subject prior to the treatment, wherein a lower methylation level of one or more of the target marker(s) relative to its corresponding methylation level prior to the treatment indicates that the subject is responsive to the treatment.

In some embodiments, the set of target markers of the present disclosure comprises 2, 3, 4, 5, 6, 7, 8, 9, 10, 11, 12, 13, 14, 15, 16, 17, 18, 19, 20, 21, 22, 23, 24, 25, 26, 27, 28 or more target markers.

In some embodiments, the step (II) of the present disclosure comprises:
(i) pre-amplifying at least a portion of at least one target marker of a set of target markers within the treated DNA obtained from step (I) with a pre-amplification primer pool, and the set of target markers are selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(ii) quantifying individual methylation level of the set of target markers within achieved DNA from the said sub-step (i).

In some embodiments, the sub-step (i) of step (II) is present. In some embodiments, the sub-step (i) of step (II) is absent. In some embodiments, the method described above further comprises obtaining DNA from a biological sample from a subject before the step (I).

In another aspect, the present disclosure provides a method of diagnosing colorectal neoplasm, screening for the onset or risk to the onset of colorectal neoplasm or assessing the development or prognosis of colorectal neoplasm in a subject, said method comprising the following steps:
(a) obtaining a biological sample containing DNA from the subject;
(b) treating the DNA in the biological sample obtained from step (a) with a reagent capable of distinguishing between an unmethylated site and a methylated site in the DNA, thereby obtaining a treated DNA;
(c) pre-amplifying at least a portion of at least one target marker within the treated DNA obtained from step (b) with a pre-amplification primer pool, wherein at least a portion of at least one (e.g. each) of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d) if step (c) is present, then quantifying individually methylation level of the at least one (e.g. each) target marker based on achieved DNA from step (c); if step (c) is absent, then quantifying individually methylation level of at least one (e.g. each) target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(e) comparing the methylation level of at least one (e.g. each) target marker from step (d) respectively with a corresponding reference level, wherein an identical or higher methylation level of one or more of the target marker(s) relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In another aspect, the present disclosure provides a method of monitoring treatment response in a subject who is receiving treatment of colorectal neoplasm, comprising the following steps:
(a) obtaining a biological sample containing DNA from the subject;
(b) treating the DNA in the biological sample obtained from step (a) with a reagent capable of distinguishing between an unmethylated site and a methylated site in the DNA, thereby obtaining a treated DNA;
(c) pre-amplifying at least a portion of at least one target marker within the treated DNA obtained from step (b) with a pre-amplification primer pool, wherein at least a portion of at least one (e.g. each) of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d) if step (c) is present, then quantifying individually methylation level of the at least one (e.g. each) target marker based on achieved DNA from step (c); if step (c) is absent, then quantifying individually methylation level of at least one (e.g. each) target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(e) comparing the methylation level of at least one (e.g. each) target marker from step (d) respectively with a corresponding methylation level of one or more of the target marker(s) obtained from the same subject prior to the treatment which is quantified by repeating step (a), step (b), optionally step (c), and step (d) with respect to a biological sample containing DNA obtained from the subject prior to the treatment, wherein a lower methylation level of one or more of the target marker(s) relative to its corresponding methylation level prior to the treatment indicates that the subject is responsive to the treatment.

As used herein, the term “screen for”, and variations such as “screens for” or “screening for”, refers to the identification of a pathological state, disease or condition, such as identification of colorectal neoplasm, or refer to identification of a subject with colorectal neoplasm who may benefit from a particular treatment regimen. In the present disclosure, the terms “screening”, “screening for”, “diagnosing” and “diagnosis” may be used interchangeably.

As used herein, the term “neoplasm” should be understood as a reference to a lesion, tumor or other encapsulated or unencapsulated mass or other form of growth which comprises neoplastic cells. A “neoplastic cell” should be understood as a reference to a cell exhibiting abnormal growth. The term “growth” should be understood in its broadest sense and includes reference to proliferation. In this regard, an example of abnormal cell growth is the uncontrolled proliferation of a cell. Another example is failed apoptosis in a cell, thus prolonging its usual life span. The neoplastic cell may be a benign cell or a malignant cell. In some embodiments, the subject neoplasm is an adenoma or an adenocarcinoma. Without limiting the present invention to any one theory or mode of action, an adenoma is generally a benign tumor of epithelial origin which is either derived from epithelial tissue or exhibits clearly defined epithelial structures. These structures may take on a glandular appearance. It can comprise a malignant cell population within the adenoma, such as occurs with the progression of a benign adenoma or benign neoplastic legion to a malignant adenocarcinoma. In some embodiments, the neoplasm is malignant, such as a carcinoma. In some embodiments, the neoplasm is non-malignant, such as an adenoma.

As used herein, the term “colorectal neoplasm” refers to the neoplasm occurring in the colon, rectum, and/or vermiform appendix. In some embodiments, the colorectal neoplasm is a colorectal cancer, a large colorectal adenoma, and/or a sessile serrated polyp. In some embodiments, the colorectal neoplasm is pre-cancerous.

As used herein, the term “pre-cancerous” refers to the neoplasm that exhibits histologic changes which are associated with an increased risk of cancer development. Examples of such conditions include, in the context of colorectal cellular proliferative disorders, cellular proliferative disorders with a high degree of dysplasia, for example, adenomatous polyps of the colon.

As used herein, the term “onset” in the context of a neoplasm, such as adenoma or adenocarcinoma, should be understood as a reference to one or more cells of that subject exhibiting dysplasia. In this regard, the adenoma or adenocarcinoma may be well developed in that a mass of dysplastic cells has developed. Alternatively, the adenoma or adenocarcinoma may be at a very early stage in that only relatively few abnormal cell divisions have occurred at the time of diagnosis. The present disclosure also extends to the assessment of a subject's risk to the onset of a colorectal neoplasm, such as a colorectal cancer.

As used herein, the term “assess” or “assessment” refers to the capacity of discriminating between samples from subjects affected and not affected by colorectal neoplasm development or the capacity of discriminating between samples from subjects that have different stages of colorectal neoplasm development. In some embodiments, the assessment relates to the determination of whether a subject's tumor has entered into the developmental stage or whether there is a high probability that the subject's tumor has entered into the developmental stage. In some embodiments, the assessment relates to the classification of a subject's tumor (e.g. Stage I, Stage II, Stage III, Stage IV, etc.). In some embodiments, the assessment relates to the determination of whether the development of a subject's tumor has lessened or become more severe. In some embodiments, the assessment can help evaluate the likelihood of clinical benefit from a therapy. In some embodiments, the assessment may relate to whether and/or the probability that a patient will improve following a treatment, for example, treatment with a particular therapeutic agent. The assessing methods of the present disclosure can be used clinically to make treatment decisions by choosing the most appropriate treatment modalities for any particular patient. The assessing methods of the present disclosure can be valuable tools in evaluating whether long-term survival of the patient, following a therapeutic regimen, such as a given therapeutic regimen, including for example, administration of a given therapeutic agent or combination, surgical intervention, steroid treatment, etc., is likely.

The discriminating or discrimination as understood by a person skilled in the art cannot aim to be correct in 100% of the samples analyzed. However, it requires that a statistically significant quantity of the samples analyzed is correctly classified. The quantity that is statistically significant can be established by a person skilled in the art by the use of different statistical tools, for example, but without being limited to, by the determination of confidence intervals, determination of p value, Student test or Fisher's discriminating functions. Details are found in Dowdy and Wearden, Statistics for Research, John Wiley & Sons, New York 1983. In certain embodiments, the confidence intervals are at least 90%, at least 95%, at least 96%, at least 97%, at least 98% or at least 99%. In some embodiments, the p value is less than 0.1, 0.05, 0.01, 0.005 or 0.0001.

As used herein, the term “development” refers to the alteration of cell morphology and physiology along a genetically determined pathway, for example, the process of natural progression in physical maturation from a previous, lower or early stage to a later, more complex or advanced stage.

As used herein, the term “prognosis” refers to the prediction of the likelihood of outcomes of disease symptoms, including, for example, recurrence, flaring, and drug resistance, of a disease (e.g. cancer). The term also refers to the prediction of the likelihood of clinical benefit from a therapy. In some embodiments, the use of statistical algorithms provides a prognosis of a disease in a subject. For example, the prognosis can be surgery, development of a clinical subtype of cancer (e.g., solid tumors, such as colorectal cancer, melanoma, and renal cell carcinoma), development of one or more clinical factors, or recovery from the disease. The prognosis may be poor prognosis (e.g. likely to recur or develop drug resistance) or benign prognosis.

As used herein, the term “responsive” refers to a subject's beneficial response to a treatment. A subject's responsiveness to a treatment can be assessed using any endpoint indicating a benefit to the subject, including, without limitation, (1) inhibition, to some extent, of disease progression, including slowing down and complete arrest; (2) reduction in the number of disease episodes and/or symptoms; (3) reduction in lesion size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) relief, to some extent, of one or more symptoms associated with the disorder; (7) increase in the length of disease-free presentation following treatment; (8) decrease of auto-immune response, which may, but does not have to, result in the regression or ablation of the disease lesion, e.g., progression-free survival; (9) increased overall survival; (10) higher response rate; and/or (11) decreased mortality at a given point of time following treatment. The term “benefit” or “beneficial” is used in the broadest sense and refers to any desirable effect.

In the present disclosure, the detailed descriptions of step (a), step (b), step (c) and step (d) apply to both the method of diagnosing colorectal neoplasm, screening for the onset or risk to the onset of colorectal neoplasm or assessing the development or prognosis of colorectal neoplasm in a subject, and the method of monitoring treatment response in a subject who is receiving treatment of colorectal neoplasm. While step (e) for both methods will be described separately. In addition, in the present disclosure, step (I) of the present disclosure is identical or at least similar to step (b) of the present disclosure. In addition, sub-step (i) of step (II) of the present disclosure is identical or at least similar to step (c) of the present disclosure; sub-step (ii) of step (II) of the present disclosure is identical or at least similar to step (d) of the present disclosure. In addition, step (III) of the present disclosure is identical or at least similar to step (e) of the present disclosure. Accordingly, step (I) and step (b) are collectively described as “step (b)” below, sub-step (i) of step (II) and step (c) are collectively described as “step (c)” below, sub-step (ii) of step (II) and step (d) are collectively described as “step (d)” below, and step (III) and step (e) are collectively described as “step (e)” below.

### Step (a)

In step (a) of the methods according to the present disclosure, a biological sample containing DNA from the subject is obtained.

As used herein, the term “biological sample” refers to a biological composition that is obtained or derived from a subject of interest that contains a cellular and/or other molecular entity (e.g. DNA) that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is selected from the group consisting of a tissue section, biopsy, a paraffin-embedded tissue, a body fluid, colonic effluent, a surgical resection sample, an isolated blood cell, a cell isolated from blood, and any combination thereof. In some embodiments, the body fluid is selected from the group consisting of whole blood, blood serum, blood plasma, urine, mucus, saliva, peritoneal fluid, pleural fluid, chest fluid, synovial fluid, cerebrospinal fluid, thoracentesis fluid, abdominal fluid, and any combination thereof. In some embodiments, the colonic effluent is selected from the group consisting of a stool sample and an enema wash sample. The choice of what type of sample is most suitable for testing in accordance with the method disclosed herein will be dependent on the nature of the situation. In some embodiments, the biological sample is obtained from whole blood of the subject. In some embodiments, the biological sample is obtained from blood plasma of the subject. A person skilled in the art will recognize various methods to prepare blood plasma from whole blood. For example, in some embodiments, the blood plasma is obtained by one, two, three, four, five or more times of centrifugation of whole blood from the subject.

As used herein, the term “subject” includes both human and non-human animals. Non-human animals include all vertebrates, such as mammals and non-mammals. The “subject” may also be a domestic animal such as cow, swine, sheep, poultry and horse; or rodent such as rat, mouse; or a non-human primate such as ape, monkey, rhesus monkey; or domesticated animal such as dog or cat. In some embodiments, the subject is a human or non-human primate. In some embodiments, the subject is a human. The terms “subject” and “individual” may be used interchangeably in the present disclosure.

In some embodiments, the DNA is isolated from the biological sample. The isolation and purification of DNA from biological samples can be performed by using various methods known in the art, including the use of commercially available kits. For example, DNA is isolated from cells and tissues by lysing the starting materials under highly denaturing and reducing conditions, partly using protein-degrading enzymes, purifying the nucleic acid fractions obtained by means of phenol/chloroform extraction processes and recovering the nucleic acids from the aqueous phase by dialysis or ethanol precipitation (see e.g. Sambrook, J., Fritsch, E. F. in T. Maniatis, C S H, Molecular Cloning, 1989). For another example, there are now a number of reagent systems, particularly for purifying DNA fragments from agarose gels and for isolating plasmid DNA from bacterial lysates, but also for isolating longer-chained nucleic acids (genomic DNA, total cell RNA) from blood, tissues or cell cultures. Many of these commercially available purification systems are based on the reasonably well known principle of binding nucleic acids to mineral carriers in the presence of solutions of different chaotropic salts. In these systems, suspensions of finely ground glass powder, diatomaceous earth or silica gels are used as carrier materials. Some other methods for isolating and purifying DNA from biological samples are described in, for example, U.S. Pat. No. 7,888,006B2 and EP1626085A1. The choice of method will be affected by several factors including time, expense and required quantity of DNA.

In some embodiments, the DNA contained in the biological sample comprises genomic DNA. As used herein, the term “genomic DNA” refers to DNA containing a complete genome of a cell or organism, and fragments or portions thereof. Genomic DNA are large pieces of DNA (e.g. longer than about 10, 20, 30, 40, 50, 60, 70, 80, 90, 100, 200, or 300 kb) derived from the subject, and can have natural modifications such as DNA methylation.

In some embodiments, the DNA contained in the biological sample comprises cellular DNA. As used herein, the term “cellular DNA” refers to DNA existing in a cell in vivo, or DNA that has been obtained from the in vivo cell and separated, isolated or otherwise manipulated in vitro so long as the DNA was not removed from the cell in vivo.

In some embodiments, the DNA contained in the biological sample comprises cell-free DNA. As used herein, the term “cell-free DNA” refers to DNA fragments existing outside of cells in vivo. The term can also be used to refer to the DNA fragments that have been obtained from the in vivo extracellular sources and separated, isolated or otherwise manipulated in vitro. The DNA fragments in cell-free DNA typically have length ranging about 100 to 200 bp, which presumably relates to the length of a DNA stretch wrapped around a nucleosome. Cell-free DNA includes, for example, cell-free fetal DNA and circulating tumor DNA. Cell-free fetal DNA circulates in the body, such as in the blood, of a pregnant mother, and represents the fetal genome, while circulating tumor DNA circulates in the body, such as in the blood, of a cancer patient. In some embodiments, the cell-free DNA may be substantially free of cellular DNA of the subject. For example, the cell-free DNA may contain less than about 1,000 ng per mL, less than about 100 ng per mL, less than about 10 ng per mL, or less than about 1 ng per mL, of cellular DNA.

The cell-free DNA may be prepared by using conventional techniques known in the art. For example, cell-free DNA of a blood sample may be obtained by centrifuging the blood sample for about 3-30 min, for about 3-15 min, for about 3-10 min, for about 3-5 min, at a speed of about 200-20,000 g, about 200-10,000 g, about 200-5,000 g, about 300-4000 g, etc. For example, in some embodiments, the cell-free DNA of a blood sample may be obtained by one, two, three, four, five or more times of centrifugation of blood plasma or serum from the subject. In some embodiments, the biological sample may be obtained by microfiltration in order to separate the cells and their fragments from a cell-free fraction comprising soluble DNA. Conventionally, microfiltration may be carried out using a filter, for example, 0.1 μm˜0.45 μm membrane filter, such as 0.22 μm membrane filter.

In some embodiments, extraction of cell-free DNA from whole blood, blood serum or blood plasma for analysis is performed using commercially available DNA extraction products. Such extraction methods claim high recoveries of circulating DNA (>50%) and some products (for example; the QIAamp Circulating Nucleic Acid Kit produced by Qiagen) are claimed to extract DNA fragments of small size. Typical sample volumes used are in the range 1-5 mL of serum or plasma.

In some embodiments, the cell-free DNA comprises circulating tumor DNA. Circulating tumor DNA (“ctDNA”) is tumor-derived fragmented DNA in body fluids (e.g. blood, urine, saliva, sputum, stool, pleural fluid, cerebrospinal fluid, etc.) that is not associated with cells. Usually, ctDNA is highly fragmented, with an average length of approximately 150 base pairs. ctDNA generally comprises a very small fraction of the cell-free DNA in the body fluids (e.g. plasma), for example, ctDNA may constitute less than about 10% of the plasma DNA. Generally, this percentage is less than about 1%, for example, less than about 0.5% or less than about 0.01%. Additionally, the total amount of plasma DNA is generally very low, for example, at about 10 ng/mL of plasma. The quantity of ctDNA varies among individuals and depends on the type of tumor, its location, and for cancerous tumors, the cancer stage. However, ctDNA is usually very rare in body fluids and can only be detected by extremely sensitive and specific techniques. The detection of ctDNA may be helpful in detecting and diagnosing a tumor, guiding tumor-specific treatment, monitoring treatment, and monitoring the remission of a cancer.

### Step (b)

In step (b) of the methods according to the present disclosure, the DNA in the biological sample obtained from step (a) is treated with a reagent capable of distinguishing between an unmethylated site and a methylated site in the DNA, thereby obtaining a treated DNA.

DNA methylation is a biological process by which methyl groups are added (for example, by the action of a DNA methyl transferase enzyme) to the DNA molecule (for example, to a cytosine base or bases of the DNA molecule). In mammals, DNA methylation is almost found at the 5′ position of a cytosine-phosphate-guanine (CpG) dinucleotides (i.e. “CpG site”), which leads to epigenetic inactivation of genes when found in 5′-CpG-3′ dinucleotides within promoters or in the first exon of genes. It is well demonstrated that DNA methylation plays an important role in the regulation of gene expression, tumorigenesis, and other genetic and epigenetic diseases.

As used herein, the term “methylated cytosine residue” refers to the derivative of a cytosine residue whereby a methyl group is attached to the carbon atom (e.g. C5 atom) of the cytosine ring. The term “unmethylated cytosine residue” refers to an underivatized cytosine residue whereby no methyl group is attached to the carbon atom (e.g. C5 atom) of the cytosine ring in contrast to the “methylated cytosine residue”. A CpG site in which the cytosine residue is methylated is a methylated CpG site, whereas a CpG site in which the cytosine residue is not methylated is an unmethylated CpG site.

In some embodiments, the reagent used in step (b) is capable of distinguishing between unmethylated and methylated CpG site(s) in the DNA, thereby obtaining a treated DNA. The reagent may selectively act on unmethylated cytosine residue(s) but not significantly act on methylated cytosine residue(s); or the reagent may selectively act on methylated cytosine residue(s) but not significantly act on unmethylated cytosine residue(s). Consequently, the original DNA is converted to a treated DNA in a methylation dependent manner, such that the treated DNA could be distinguished from the original DNA by its hybridization behavior.

For example, some reagents may selectively convert unmethylated cytosine residue(s) into uracil, thymine, or another base that is dissimilar to cytosine in terms of hybridization, while methylated cytosine residue(s) remained unconverted. For another example, some reagents may selectively cleave at a residue when it is methylated, or selectively cleave at a residue when it is unmethylated.

As used herein, the term “treated DNA” refers to the DNA that has been treated with a reagent which is capable of distinguishing between an unmethylated site and a methylated site in the DNA, i.e. the DNA methylation status in the DNA has been changed.

In certain embodiments, the reagent of step (b) selectively modifies at unmethylated cytosine residue(s) at the CpG site(s) to produce modified residue(s) but does not significantly modify methylated cytosine residue(s).

In some embodiments, the reagent of step (b) comprises a bisulfite reagent. As used herein, the term “bisulfite reagent” refers to a reagent comprising bisulfite, disulfite, hydrogen sulfite or any combination thereof, useful as disclosed herein to distinguish between methylated and unmethylated CpG dinucleotide sequences. In the present disclosure, the treatment of DNA with a bisulfite reagent is also described as a “bisulfite reaction” or “bisulfite treatment”, which means a reaction for the conversion of a unmethylated cytosine residue, in particular unmethylated cytosine residues, in a nucleic acid to uracil base(s), thymine base(s) or other base(s) which is(are) dissimilar to cytosine(s) in terms of hybridization behavior, in the presence of bisulfite ions whereby methylated cytosine residues are not significantly converted. In other words, the bisulfite treatment is useful for distinguishing between methylated and unmethylated CpG dinucleotides.

The bisulfite reaction for the detection of methylated cytosine residues is described in detail by Frommer, M., et al., Proc Natl Acad Sci USA 89 (1992) 1827-31 and Grigg, G., and Clark, S., Bioessays 16 (1994) 431-6. The bisulfite reaction contains a deamination step and a desulfonation step (see Grigg and Clark, supra). The statement that methylated cytosine residues are not significantly converted shall only take the fact into account that it cannot be excluded that a very small percentage (for example, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, less than 10%, less than 110, less than 12%, less than 13%, less than 14%, less than 15%, less than 16%, less than 17%, less than 18%, less than 19%, less than 20%) of methylated cytosine residues is converted to uracil, thymine, or another base which is dissimilar to cytosine in terms of hybridization behavior, although it is intended to convert only and exclusively the unmethylated cytosine residues.

A person skilled in the art knows how to perform the bisulfite treatment, in particular the deamination step and the desulfonation step, e.g. by referring to Frommer M., et al. supra or Grigg and Clark, supra who disclose the principal parameters of the bisulfite treatment. The influence of incubation time and temperature on deamination efficiency and parameters affecting DNA degradation is disclosed.

In some embodiments, the bisulfite reagent is selected from the group consisting of ammonium bisulfite, sodium bisulfite, potassium bisulfite, calcium bisulfite, magnesium bisulfite, aluminum bisulfite, hydrogen sulfite and any combination thereof. In some embodiments, the bisulfite reagent is sodium bisulfite. In some embodiments, the bisulfite reagent is commercially available, for example, MethylCode™ Bisulfite Conversion Kit, EpiMark™ Bisulfite Conversion Kit, EpiJET™ Bisulfite Conversion Kit, EZ DNA Methylation-Gold™ Kit, etc. In some embodiments, the bisulfite reaction is performed according to the use instructions of the kits.

In some embodiments, the reagent of step (b) selectively cleaves at a residue when it is unmethylated but does not cleave at the residue when it is methylated, or selectively cleaves at the residue when it is methylated but does not cleave at the residue when it is unmethylated.

In some embodiments, the reagent of step (b) is a methylation sensitive restriction enzyme (MSRE).

The term “methylation sensitive restriction enzyme” refers to an enzyme that selectively digests a nucleic acid dependent on the methylation state of its recognition site. In the case of such restriction enzymes which specifically cut if the recognition site is not methylated or hemimethylated, the cut will not take place or with a significantly reduced efficiency if the recognition site is methylated. In the case of such restriction enzymes which specifically cut if the recognition site is methylated, the cut will not take place or with a significantly reduced efficiency if the recognition site is not methylated. In some embodiments, the recognition sequence of the methylation sensitive restriction enzymes contains a CG dinucleotide (for instance cgcg or cccggg). In some embodiments, the methylation sensitive restriction enzymes do not cut if the cytosine residue in this CG dinucleotide is methylated at the carbon atom C5.

In some embodiments, the MSRE is selected from the group consisting of HpaII, SalI, SalI-HF®, ScrFI, BbeI, Nod, SmaI, XmaI, MboI, BstBI, ClaI, MluI, NaeI, NarI, PvuI, SacII, HhaI and any combination thereof.

Methods are known in the art wherein a methylation sensitive restriction enzyme, or a series of restriction enzyme reagents comprising methylation sensitive restriction enzymes that distinguish between methylated and non-methylated CpG dinucleotides within a target region are utilized in determining methylation, for example but not limited to differential methylation hybridization (“DMH”).

In some embodiments, the DNA of step (a) may be cleaved prior to treatment with methylation sensitive restriction enzymes. Such methods are known in the art and may include both physical and enzymatic means. Particularly preferred is the use of one or a plurality of restriction enzymes which are not methylation sensitive, and whose recognition sites are AT rich and do not comprise CG dinucleotides. The use of such enzymes enables the conservation of CpG sites and CpG rich regions in the fragmented DNA. In some embodiments, such restriction enzyme is selected from the group consisting of MseI, BfaI, Csp6I, TrulI, Tru9I, MaeI. XspI and any combination thereof.

### Step (c)

In step (c) of the methods according to the present disclosure, at least one target marker within the treated DNA obtained from step (b) are pre-amplified with a pre-amplification primer pool, wherein at least a portion of at least one (e.g. each) of the target marker(s) is pre-amplified. In the present disclosure, step (c) may be also designated as a pre-amplification step. While not wishing to be bound by any theory, it is believed that step (c) is not necessarily required to achieve the purpose of the present invention. In some embodiments, step (c) of the methods according to the present disclosure is present. In some embodiments, step (c) of the methods according to the present disclosure is absent.

One of the purposes of the pre-amplification of target marker(s) is to increase amount(s) of target marker(s) within the treated DNA, e.g. from low amount(s) of target marker(s). As used herein, the term “amplification”, and variations such as “amplifying”, “amplified” and “amplifies”, refer generally to any process that results in an increase in the copy number of a molecule or set of related molecules. As it applies to polynucleotide molecules, amplification means the production of multiple copies of a polynucleotide molecule, or a portion of a polynucleotide molecule, typically starting from a small amount of a polynucleotide, where the amplified material (amplicon, PCR amplicon) is typically detectable. Amplification of polynucleotides encompasses a variety of chemical and enzymatic processes. The generation of multiple DNA copies from one or a few copies of a template RNA or DNA molecule during a polymerase chain reaction (reverse transcription PCR, PCR), a strand displacement amplification (SDA) reaction, a transcription mediated amplification (TMA) reaction, a nucleic acid sequence-based amplification (NASBA) reaction, or a ligase chain reaction (LCR) are forms of amplification.

As used herein, the term “target marker” refers to a nucleic acid, or a gene region of interest, whose methylation level is indicative for colorectal neoplasm (e.g. colorectal cancer), or indicative for the onset or risk to the onset of colorectal neoplasm (e.g. colorectal cancer), or indicative for the development or prognosis of colorectal neoplasm (e.g. colorectal cancer). The terms “marker” and “gene” may be used interchangeably in the present disclosure. The term “marker” or “gene” shall be taken to include all transcript variants thereof (e.g. the term “Septin9” shall include for example its truncated transcript Q9HC74) and all promoter and regulatory elements thereof. As would be appreciated by a person of skill in the art, some genes are known to exhibit allelic variation between subjects or single nucleotide polymorphisms (“SNPs”). SNPs encompass insertions and deletions of varying size and simple sequence repeats, such as dinucleotide and trinucleotide repeats. The present disclosure should therefore be understood to extend to all forms of markers/genes which arise from any other mutations, polymorphic or allelic variations. In addition, it should be understood that the terms “marker” and “gene” shall include sequences of both the sense strand and antisense strand of the marker or gene.

The term “target marker” as used herein is broadly construed to encompass both 1) the original marker (in a particular methylation status) found in the biological sample or in genomic DNA, and 2) the treated sequence thereof (for example a bisulfite converted counterpart or a MSRE treated counterpart). A bisulfite converted counterpart differs from the target marker in the genomics sequence in that one or more unmethylated cytosine residues are converted to uracil base(s), thymine base(s) or other base(s) which is(are) dissimilar to cytosine(s) in terms of hybridization behavior. A MSRE treated counterpart differs from the target marker in the genomics sequence in that the sequence are cleaved at one or more MSRE cleavage sites.

In some embodiments, the at least one target marker comprises one or multiple markers (e.g. at least 2, 3, 4, 5, 6, 7, 8, 9, 10, 11, 12, 13, 14, 15, 16, 17, 18, 19, 20, 21, 22, 23, 24, 25, 26, 27, 28 markers) selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one target marker comprises 14 markers selected from the group consisting of NDRG4, Septin9, BCAT1, IKZF1, BCAN, VAV3, IRF4, POU4F2, SALL1, PKNOX2, SDC2, ASCL4, TMEFF2 and INTERGENIC REGION 1. In some embodiments, the at least one target marker comprises 13 markers selected from the group consisting of NDRG4, Septin9, BCAT1, IKZF1, BCAN, VAV3, POU4F2, SALL1, PKNOX2, SDC2, ASCL4, TMEFF2 and INTERGENIC REGION 1. In some embodiments, the at least one target marker comprises 11 markers selected from the group consisting of Septin9, BCAT1, IKZF1, VAV3, IRF4, BCAN, NDRG4, SDC2, PKNOX2, TMEFF2, and INTERGENIC REGION 1. In some embodiments, the at least one target marker comprises 10 markers selected from the group consisting of Septin9, BCAT1, IKZF1, VAV3, BCAN, NDRG4, SDC2, PKNOX2, TMEFF2, and INTERGENIC REGION 1. In some embodiments, the at least one target marker comprises 10 markers selected from the group consisting of Septin9, BCAT1, IKZF1, VAV3, IRF4, BCAN, NDRG4, SDC2, PKNOX2, and TMEFF2. In some embodiments, the at least one target marker comprises 9 markers selected from the group consisting of Septin9, BCAT1, IKZF1, VAV3, BCAN, NDRG4, SDC2, PKNOX2, and TMEFF2. In some embodiments, the at least one target marker comprises 7 markers selected from the group consisting of Septin9, BCAT1, IKZF1, VAV3, IRF4, BCAN, and NDRG4. In some embodiments, the at least one target marker comprises 6 markers selected from the group consisting of Septin9, BCAT1, IKZF1, VAV3, BCAN, and NDRG4. In some embodiments, the at least one target marker comprises 6 markers selected from the group consisting of Septin9, BCAT1, IKZF1, VAV3, IRF4, and BCAN. In some embodiments, the at least one target marker comprises 5 markers selected from the group consisting of Septin9, BCAT1, IKZF1, VAV3, and BCAN. In some embodiments, the at least one target marker comprises 5 markers selected from the group consisting of Septin9, BCAT1, IKZF1, VAV3, and IRF4. In some embodiments, the at least one target marker comprises 3 markers selected from the group consisting of SALL1, BCAT1, and Septin9.

In some embodiments, the at least one target marker can be up to one target marker (i.e. one marker but no more than one marker). In some embodiments, the at least one target marker is Septin9. In some embodiments, the at least one target marker is BCAT1. In some embodiments, the at least one target marker is IKZF1. In some embodiments, the at least one target marker is NDRG4. In some embodiments, the at least one target marker is BCAN. In some embodiments, the at least one target marker is PKNOX2. In some embodiments, the at least one target marker is VAV3. In some embodiments, the at least one target marker is IRF4. In some embodiments, the at least one target marker is POU4F2. In some embodiments, the at least one target marker is SALL1. In some embodiments, the at least one target marker is TMEFF2. In some embodiments, the at least one target marker is ASCL4. In some embodiments, the at least one target marker is FGF12. In some embodiments, the at least one target marker is INTERGENIC REGION 1.

In some embodiments, the at least one target marker comprises multiple target markers. In some embodiments, the multiple target markers comprise at least two or three markers selected from the group consisting of Septin9, BCAT1, and IKZF1. In some embodiments, the multiple target markers of the present disclosure further comprise one, two, three, four, or five additional markers selected from the group consisting of BCAN, PKNOX2, VAV3, NDRG4, and IRF4. In some embodiments, the multiple target markers of the present disclosure further comprise one or more (e.g. 1, 2, 3, 4, 5, 6, 7, 8, 9, 10, 11, 12, 13, 14, 15, 16, 17, 18, 19, 20) additional markers selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP.

In some embodiments, the multiple target markers of the present disclosure comprise Septin9 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, BCAT1, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, BCAT1, IKZF1, NDRG4, PKNOX2, VAV3, IRF4, or any combination thereof. In some embodiments, the at least one (e.g. at least 1, 2, or 3) additional target marker comprises NDRG4, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise BCAT1 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, Septin9, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, Septin9, NDRG4, IKZF1, PKNOX2, VAV3, IRF4, or any combination thereof. In some embodiments, the at least one (e.g. at least 1, 2, or 3) additional target marker comprises NDRG4, Septin9, and/or IKZF1. In some embodiments, the at least one (e.g. at least 1, 2, or 3) additional target marker comprises BCAN, VAV3, and/or IRF4.

In some embodiments, the multiple target markers of the present disclosure comprise IKZF1 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, Septin9, BCAT1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, Septin9, BCAT1, PKNOX2, NDRG4, VAV3, IRF4, or any combination thereof. In some embodiments, the at least one (e.g. at least 1, 2, or 3) additional target marker comprises NDRG4, Septin9, and/or BCAT1. In some embodiments, the at least one (e.g. at least 1, 2, or 3) additional target marker comprises BCAN, VAV3, and/or IRF4.

In some embodiments, the multiple target markers of the present disclosure comprise BCAN and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, NDRG4, IRF4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises NDRG4, VAV3, and/or IRF4.

In some embodiments, the multiple target markers of the present disclosure comprise VAV3 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, BCAN, PKNOX2, NDRG4, IRF4 or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, NDRG4, and/or IRF4.

In some embodiments, the multiple target markers of the present disclosure comprise IRF4 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, VAV3, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, BCAN, NDRG4, PKNOX2, VAV3 or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or NDRG4.

In some embodiments, the multiple target markers of the present disclosure comprise PKNOX2 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, VAV3, IRF4, POU4F2, SALL1, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, BCAN, VAV3, NDRG4, IRF4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise POU4F2 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, BCAN, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, NDRG4, IRF4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise SALL1 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, BCAN, POU4F2, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, NDRG4, IRF4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise TMEFF2 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, BCAN, POU4F2, PKNOX2, SDC2, ASCL4, SALL1, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, IRF4, NDRG4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise ASCL4 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, BCAN, POU4F2, PKNOX2, SDC2, TMEFF2, SALL1, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, IRF4, NDRG4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise FGF12 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, 27) additional target marker selected from the group consisting of Septin9, BCAT1, IKZF1, VAV3, IRF4, BCAN, POU4F2, PKNOX2, SDC2, TMEFF2, SALL1, SLC24A2, NDRG4, NKX2-6, KCNA6, SOX1, HS3ST2, ASCL4, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, IRF4, NDRG4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise INTERGENIC REGION 1 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, BCAN, POU4F2, PKNOX2, SDC2, TMEFF2, SALL1, SLC24A2, NDRG4, NKX2-6, KCNA6, SOX1, HS3ST2, ASCL4, KCTD8, HMX1, MARCH11, CRHBP, FGF12, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, IRF4, NDRG4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise NDRG4 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, BCAN, POU4F2, PKNOX2, SDC2, TMEFF2, SALL1, SLC24A2, NKX2-6, KCNA6, SOX1, HS3ST2, ASCL4, KCTD8, HMX1, MARCH11, CRHBP, FGF12, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, IRF4, BCAN, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In the present disclosure, it should be understood that the markers/genes at issue are described herein both by reference to their names and their chromosomal coordinates. The chromosomal coordinates are consistent with the human genome database version Hg19 which was released in February 2009 (herein referred to as “Hg19 coordinates”).

In the present disclosure, it should be understood that the target marker also includes intergenic regions, which are designated as “INTERGENIC REGION 1”, “INTERGENIC REGION 2”, “INTERGENIC REGION 3”, “INTERGENIC REGION 4”, “INTERGENIC REGION 5”, and defined by their respective chromosomal coordinates. For example, in the present disclosure, INTERGENIC REGION 1 refers to the region defined by chr6:19679885-19693988; INTERGENIC REGION 2 refers to the region defined by chr10:130082033-130087148; INTERGENIC REGION 3 refers to the region defined by chr10:133107880-133113966; INTERGENIC REGION 4 refers to the region defined by chr7:152620588-152624685; and INTERGENIC REGION 5 refers to the region defined by chr8:70945014-70949177.

In some embodiments, the respective target marker comprises or is: a) the respective region defined by Hg19 coordinates as set forth below:

| Target Marker | Hg19 Coordinate |
|---|---|
| NDRG4 | chr16: 58496750-58547532 |
| BCAT1 | chr12: 24964295-25102393 |
| IKZF1 | chr7: 50343720-50472799 |
| Septin9 | chr17: 75276651-75496678 |
| SDC2 | chr8: 97505579-97624000 |
| VAV3 | chr1: 108113782-108507766 |
| IRF4 | chr6: 391739-411447 |
| TMEFF2 | chr2: 192813769-193060435 |
| SALL1 | chr16: 51169886-51185278 |
| BCAN | chr1: 156611182-156629324 |
| POU4F2 | chr4: 147560045-147563626 |
| PKNOX2 | chr11: 125034583-125303285 |
| ASCL4 | chr12: 108168162-108170421; |
| KCNA6 | chr12: 4918342-4960277; |
| SOX1 | chr13: 112721913-112726020; |
| HS3ST2 | chr16: 22825498-22927659; |
| FGF12 | chr3: 191857184-192485553; |
| KCTD8 | chr4: 44175926-44450824; |
| HMX1 | chr4: 8847802-8873543; |
| MARCH11 | chr5: 16067248-16180871; |
| CRHBP | chr5: 76248538-76276983; |
| NKX2-6 | chr8: 23559964-23564111 |
| SLC24A2 | chr9: 19507450-19786926 |
| INTERGENIC REGION 1 | chr6: 19679885-19693988 |
| INTERGENIC REGION 2 | chr10: 130082033-130087148 |
| INTERGENIC REGION 3 | chr10: 133107880-133113966 |
| INTERGENIC REGION 4 | chr7: 152620588-152624685 |
| INTERGENIC REGION 5 | chr8: 70945014-70949177, |

and 5 kb upstream of the respective start site and 5 kb downstream of the respective end site of each region described above, or b) a bisulfite converted counterpart of a), or c) a MSRE treated counterpart of a). The specific nucleotide sequences of the Hgl9 coordinates as listed above and 5 kb upstream of the respective start site and 5 kb downstream of the respective end site of each region are available in public databases such as UCSC Genome Browser, Ensemble, and NCBI websites.

In some embodiments, the respective target marker also comprises all variants thereof. The variants include nucleic acid sequences from the same region sharing at least 90%, 91%, 92%, 93%, 94%, 95%, 96%, 97%, 98%, 99% sequence identity, i.e. having one or more deletions, additions, substitutions, inverted sequences etc. relative to the marker/gene regions described herein. Accordingly, the present disclosure should be understood to extend to such variants which achieve the same outcome despite the fact that minor genetic variations between the actual nucleic acid sequences may exist between subjects.

As used herein, the term “percent (%) sequence identity” refers to the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

In step (c) provided herein, at least a portion of at least one (e.g. each) of the target marker(s) is pre-amplified. In certain embodiments, the pre-amplified portion of the target marker is within a subregion of the target marker.

Without limiting the present disclosure to any one theory or mode of action, it is believed to be particularly useful to measure methylation level of a target marker in a subregion containing a high density of CpG dinucleotides which are frequently hypermethylated in colorectal neoplasm, such as colorectal cancer. This finding renders subregions a particularly useful target for analysis since it both simplifies the screening process due to a shorter more clearly defined region of DNA requiring analysis and, further, the fact that the results from these regions will provide a significantly more definitive result in relation to the presence, or not, of hypermethylation than would be obtained if analysis was performed across the Hg19 regions of the target markers as a whole. This finding therefore both simplifies the diagnosing, screening/monitoring process and increases the sensitivity and specificity of colorectal neoplasm diagnosis. In some embodiments, the subregion of respective target marker comprises or is: a) a sequence defined by Hg19 coordinates as set forth below:

| Target Marker | Subregion Hg19 Coordinate |
|---|---|
| NDRG4 | chr16: 58497307-58497392 |
| BCAT1 | chr12: 25102016-25102110 |
| IKZF1 | chr7: 50343793-50343896 |
| Septin9 | chr17: 75369603-75369693 |
| SDC2 | chr8: 97506253-97506331 |
| VAV3 | chr1: 108507591-108507674 |
| IRF4 | chr6: 392282-392377 |
| TMEFF2 | chr2: 193059426-193059517 |
| SALL1 | chr16: 51190041-51190146 |
| BCAN | chr1: 156611866-156611966 |
| POU4F2 | chr4: 147560088-147560191 |
| PKNOX2 | chr11: 125036431-125036547 |
| ASCL4 | chr12: 108169374-108169473 |
| KCNA6 | chr12: 4918853-4918959 |
| SOX1 | chr13: 112758808-112758890 |
| HS3ST2 | chr16: 22825783-22825873 |
| FGF12 | chr3: 192125861-192125964 |
| KCTD8 | chr4: 44449597-44449687 |
| HMX1 | chr4: 8859817-8859921 |
| MARCH11 | chr5: 16180271-16180378 |
| CRHBP | chr5: 76249633-76249729 |
| NKX2-6 | chr8: 23564141-23564235 |
| SLC24A2 | chr9: 19788670-19788750 |
| INTERGENIC REGION 1 | chr6: 19691885-19691988 |
| INTERGENIC REGION 2 | chr10: 130085033-130085148 |
| INTERGENIC REGION 3 | chr10: 133110880-133110966 |
| INTERGENIC REGION 4 | chr7: 152622588-152622685 |
| INTERGENIC REGION 5 | chr8: 70947014-70947177, |

and 5 kb upstream of the respective start site and 5 kb downstream of the respective end site of each region described above, or b) a bisulfite converted counterpart of a), or c) a MSRE treated counterpart of a).

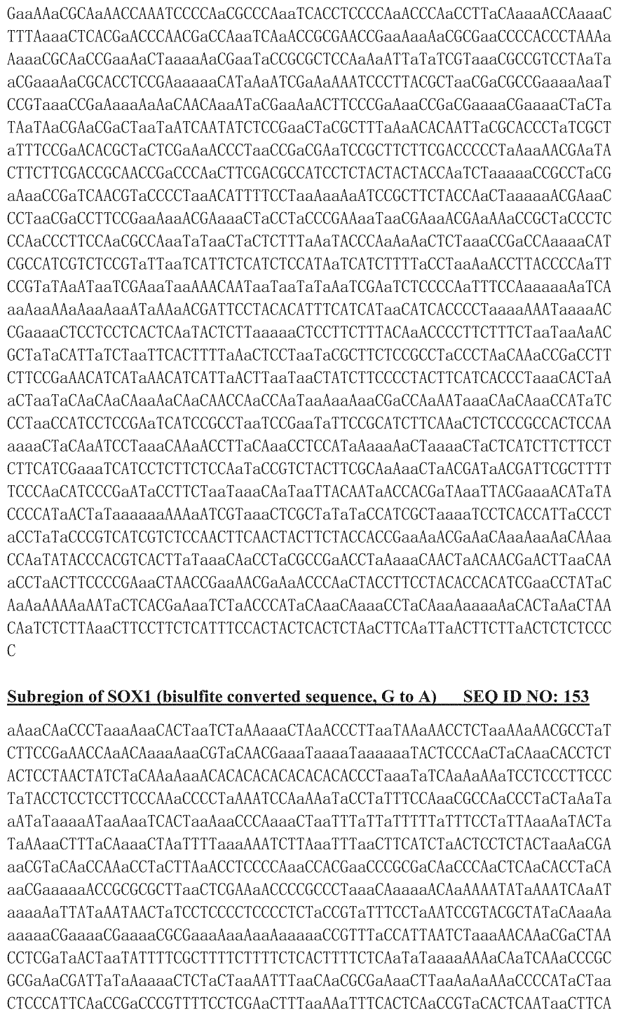

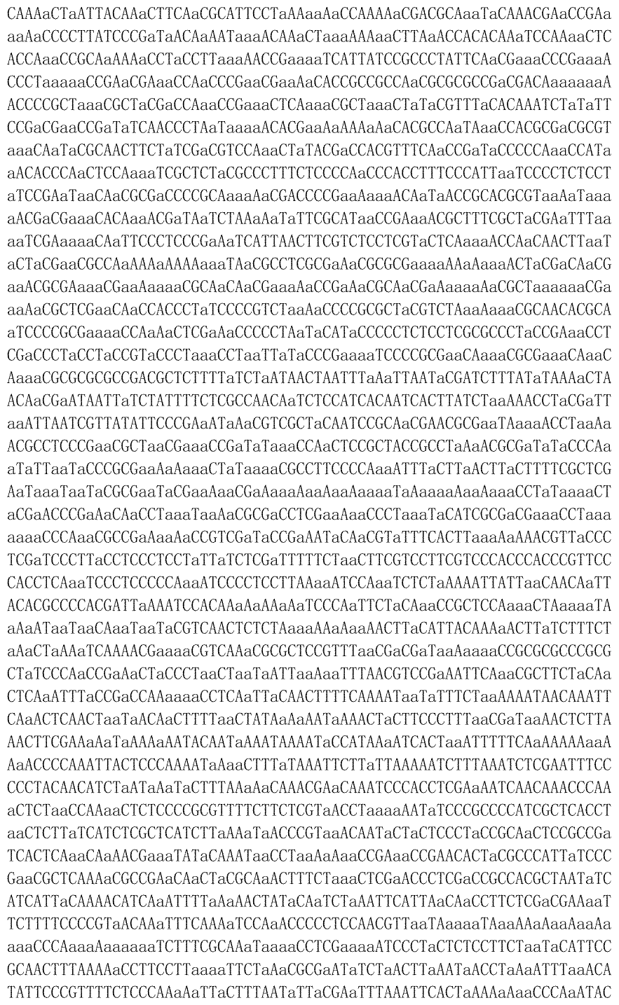

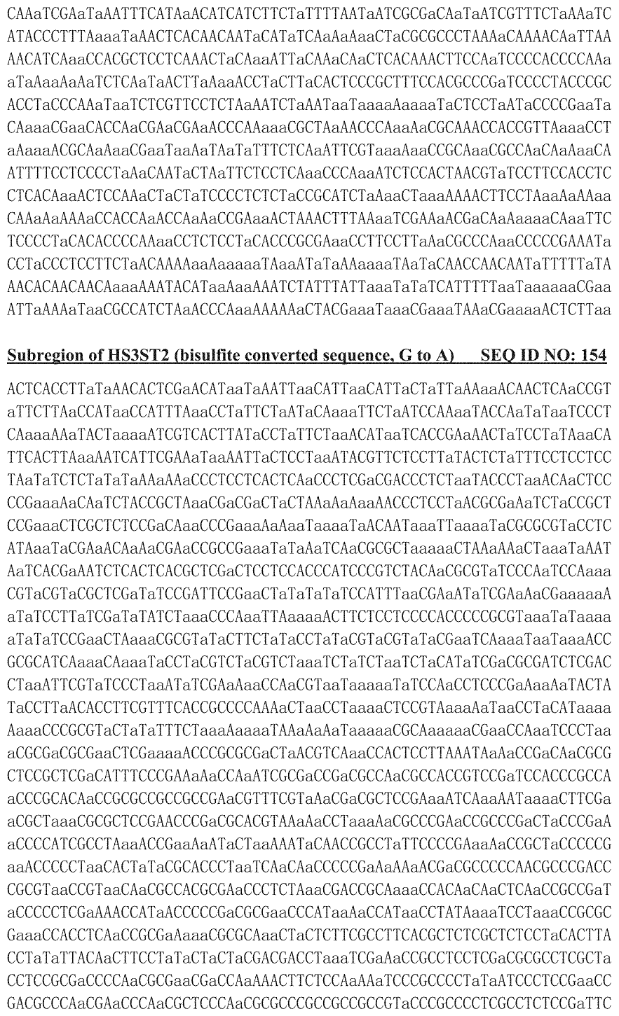

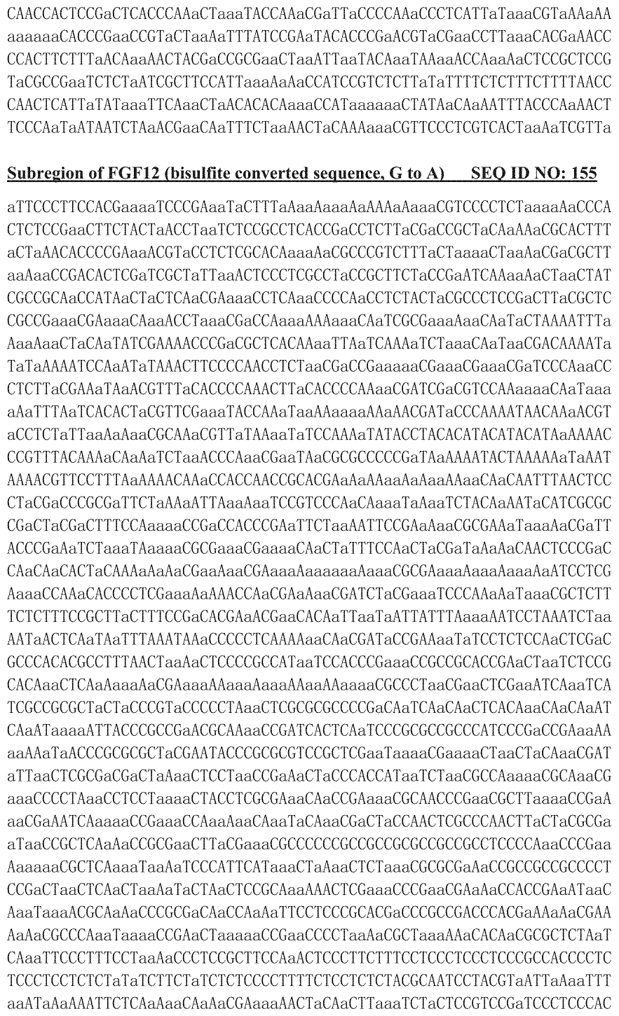

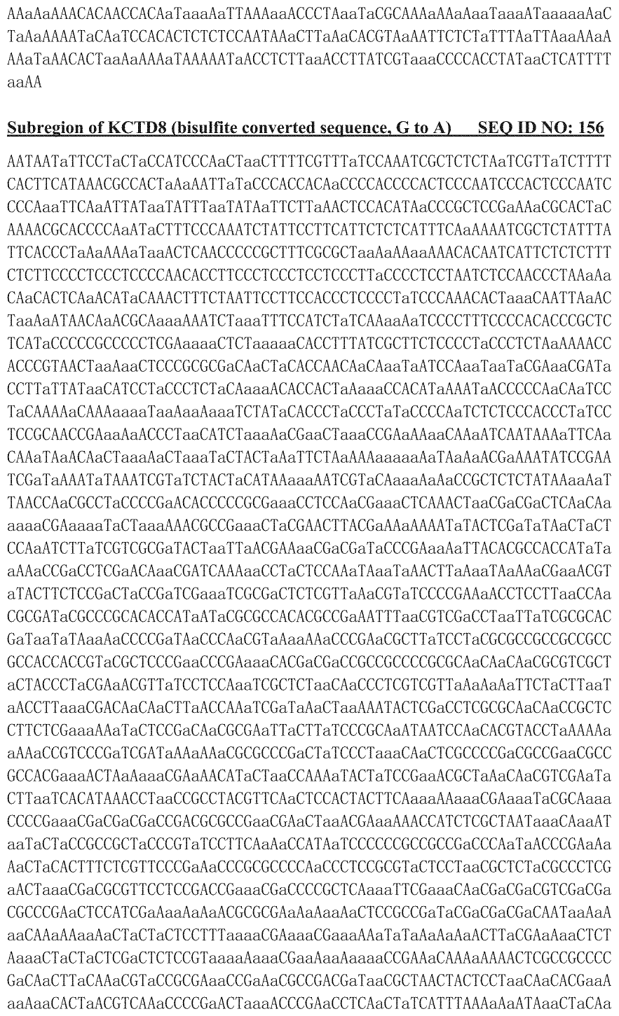

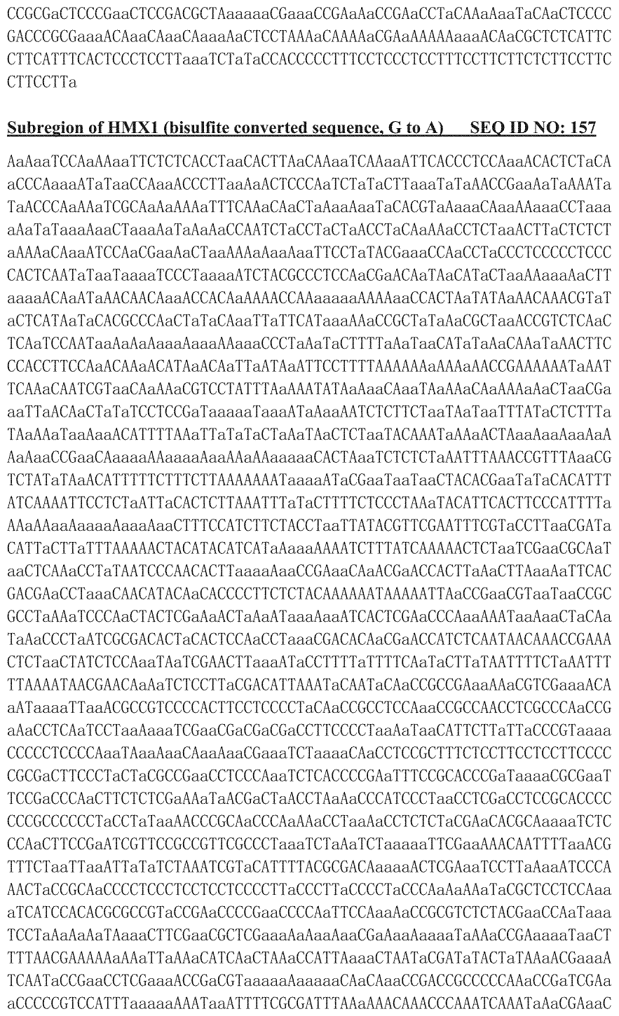

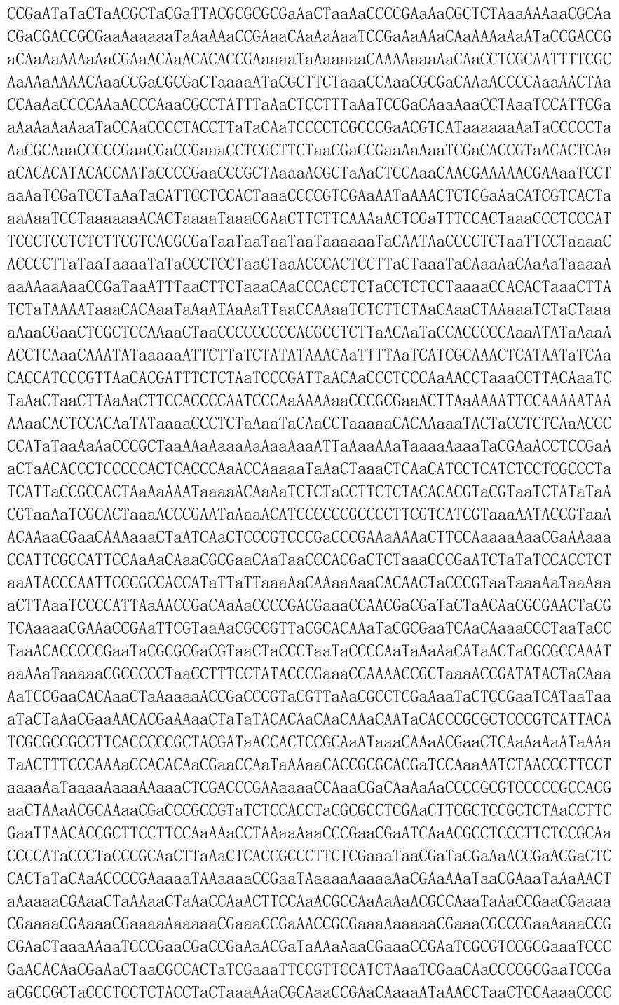

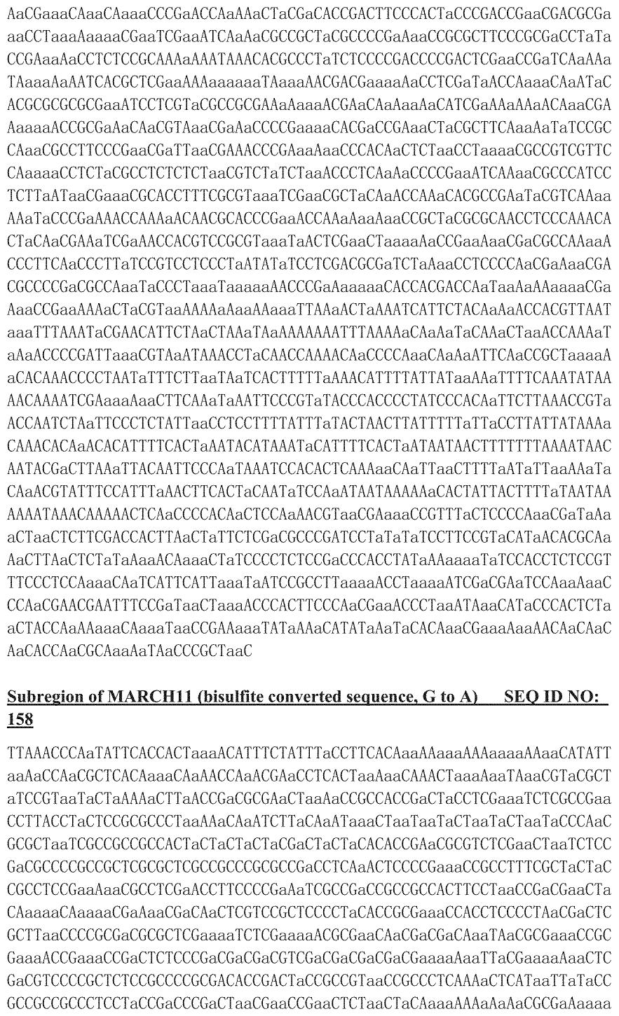

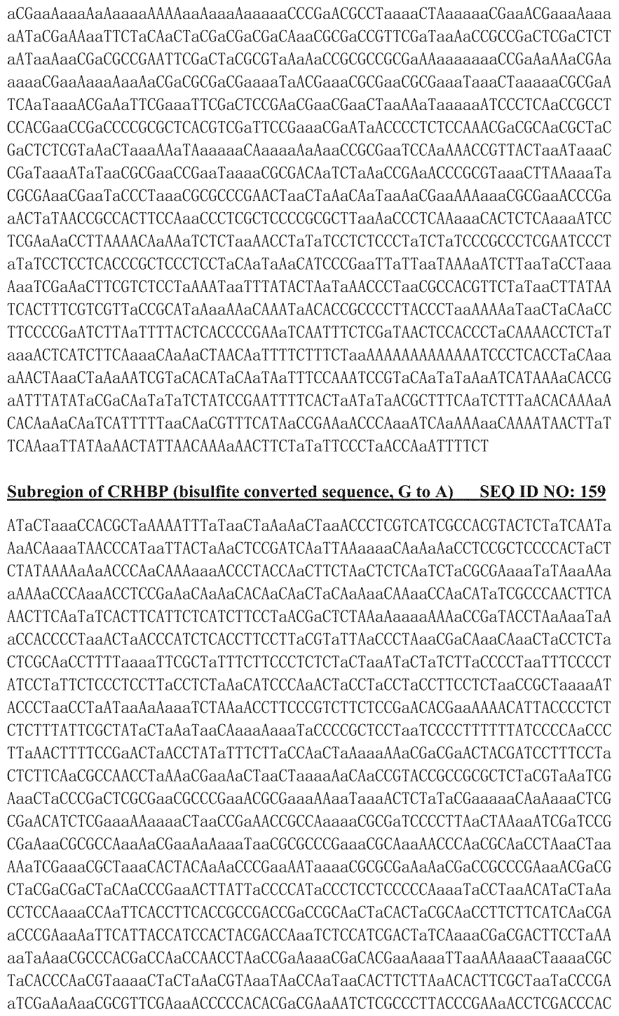

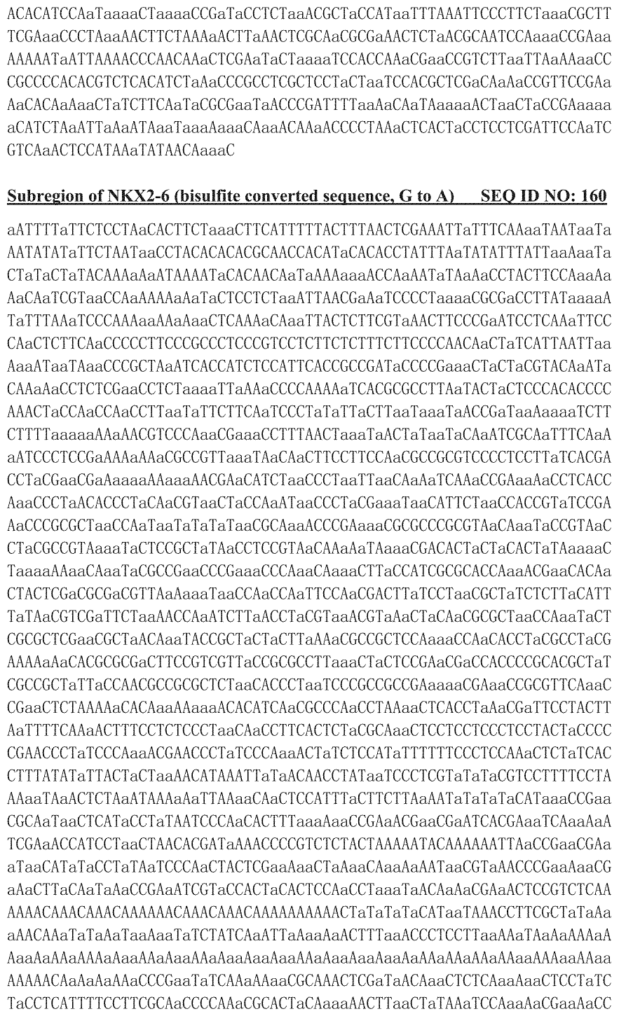

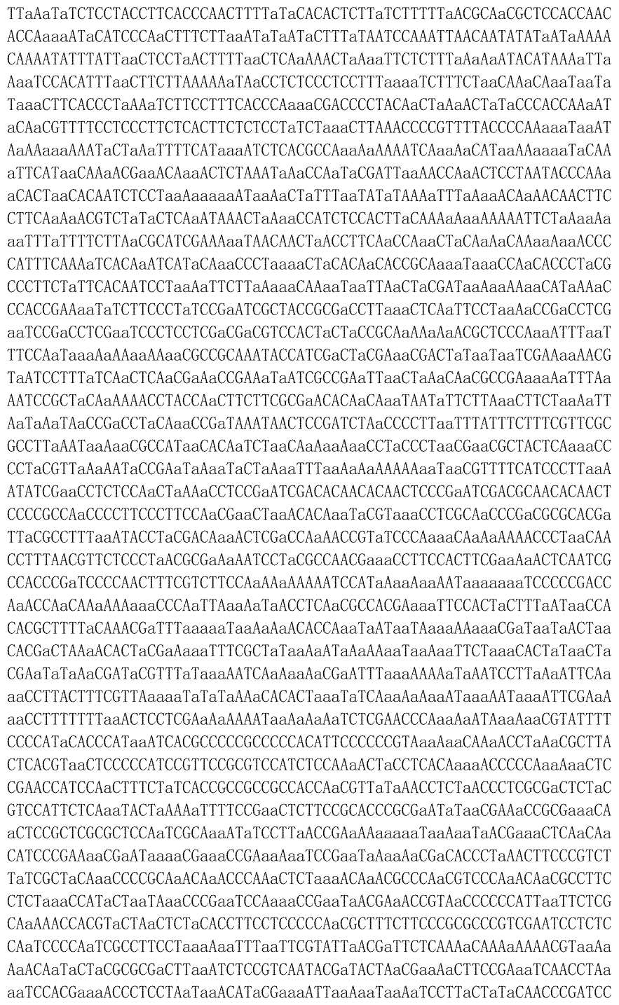

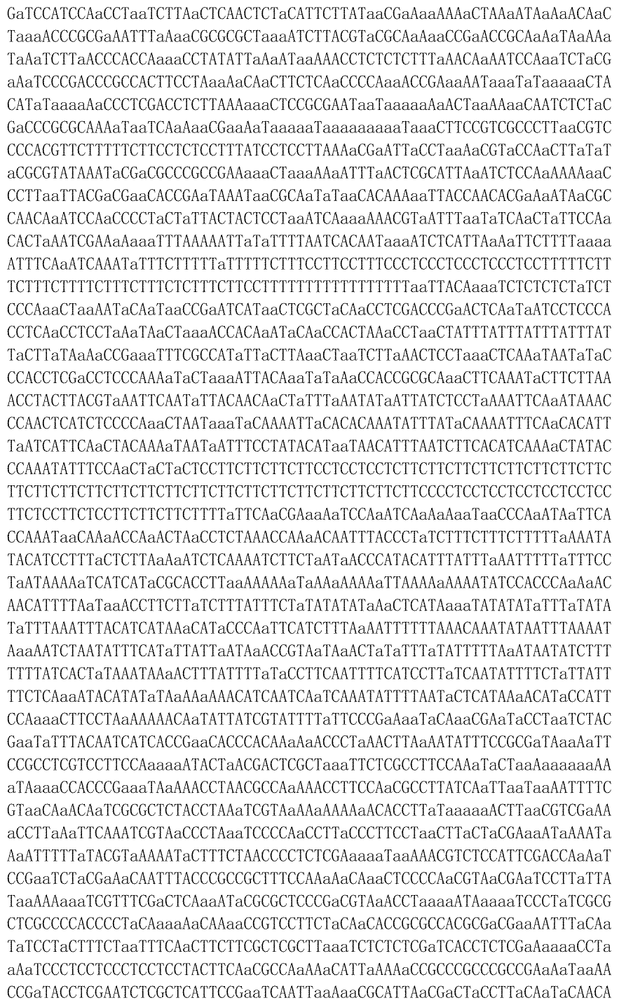

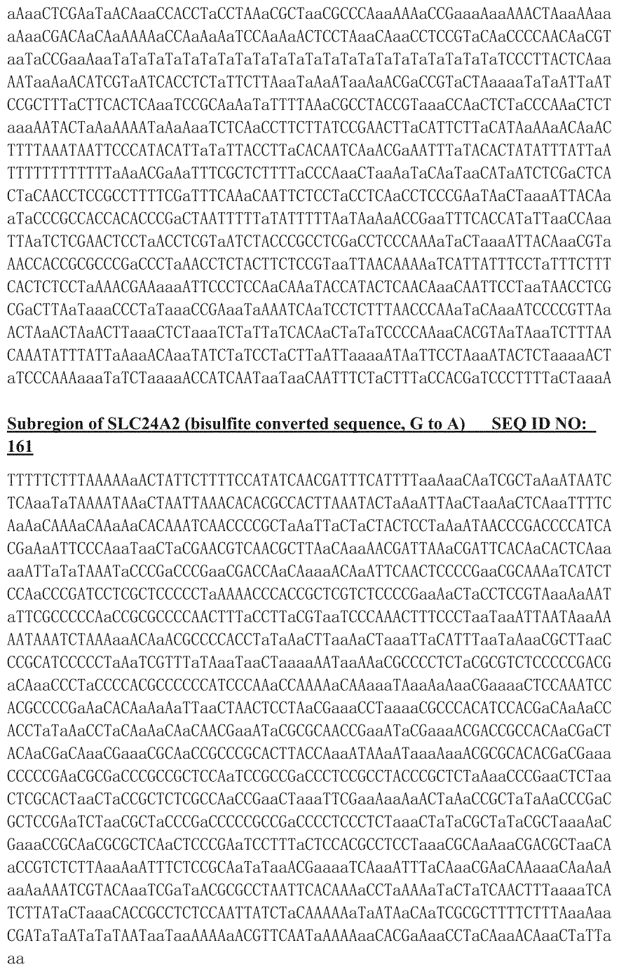

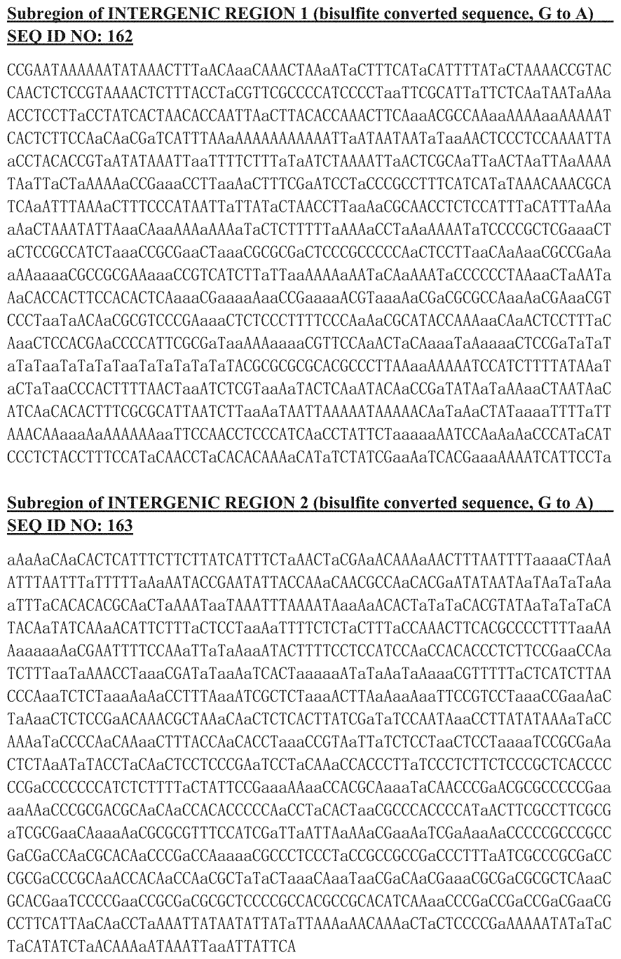

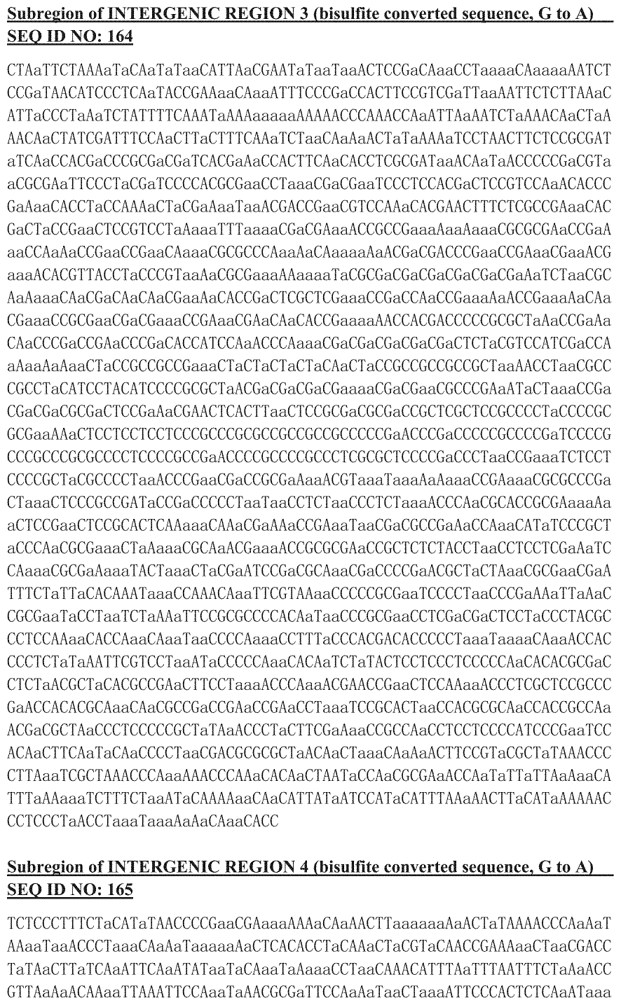

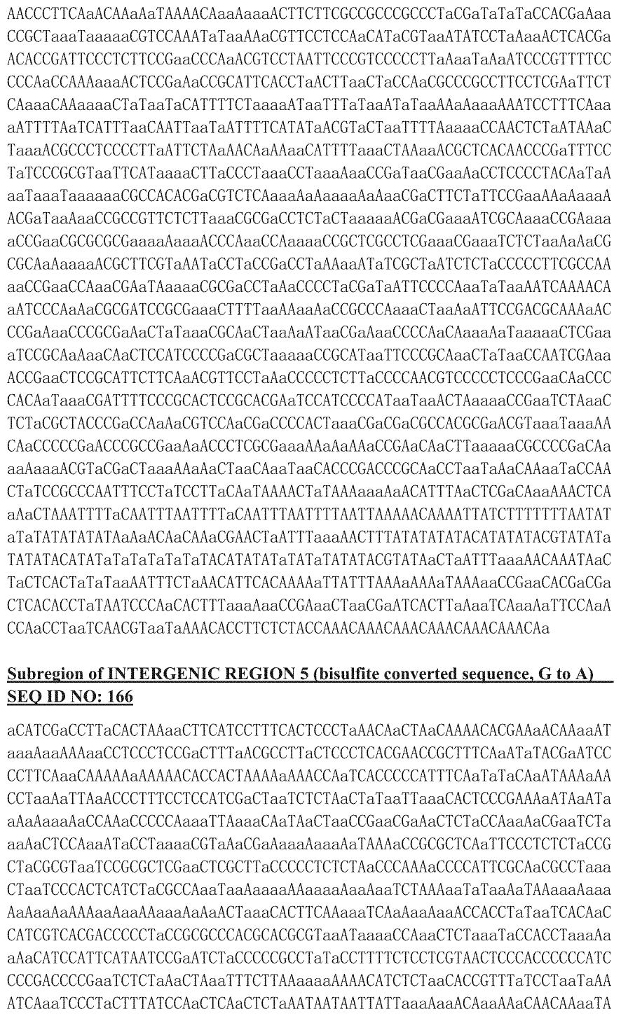

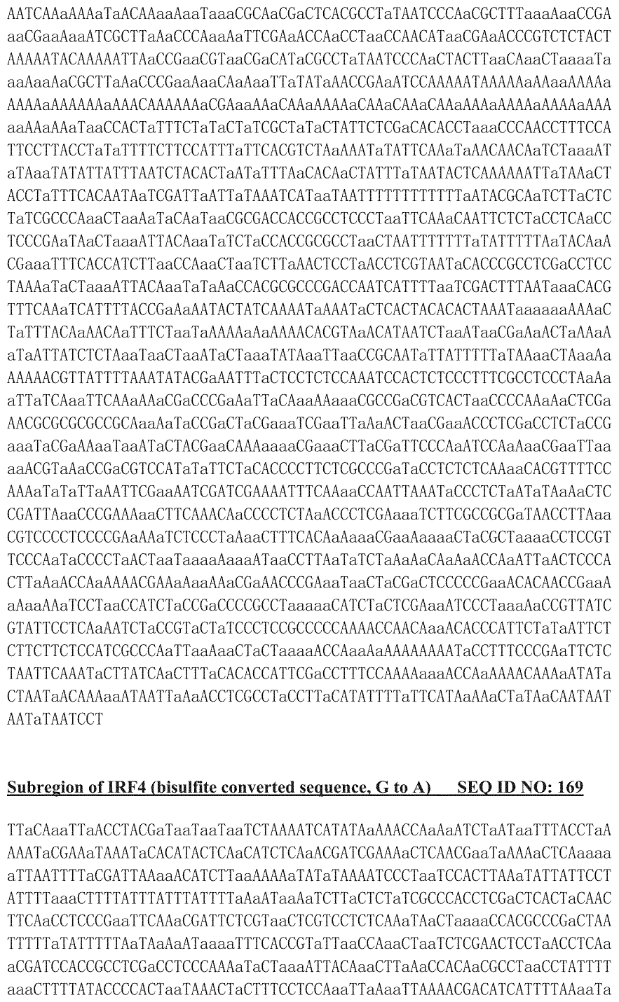

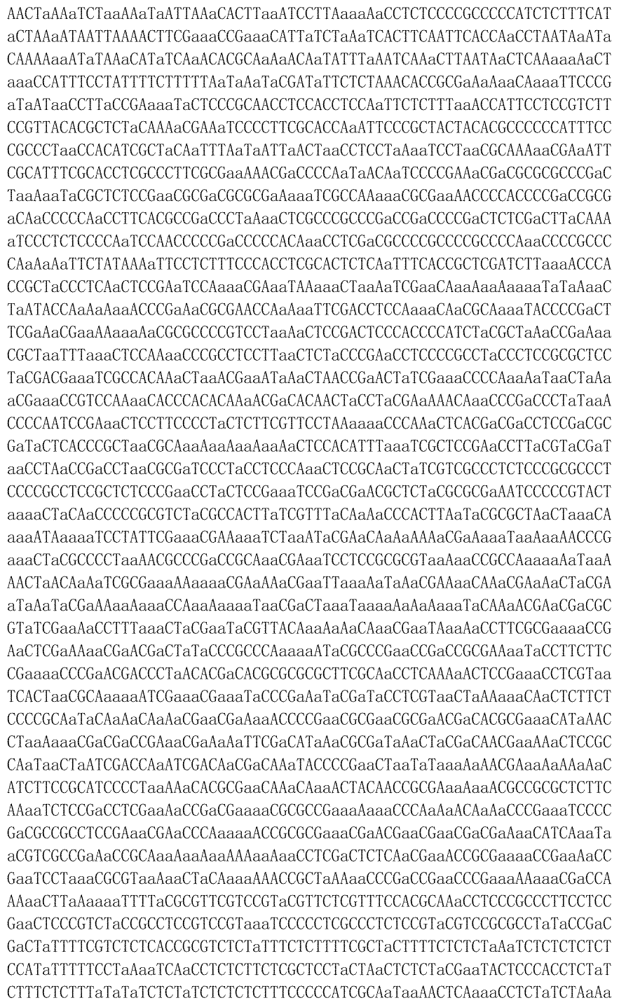

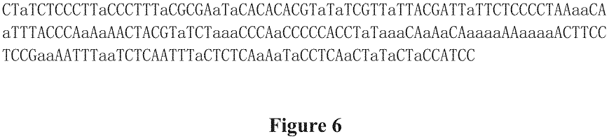

In certain embodiments, the subregion of respective target marker comprises or is a polynucleotide sequence selected from the group consisting of SEQ ID NOs: 86-112, 167, or a bisulfite converted counterpart thereof, or a MSRE treated counterpart thereof. In certain embodiments, the bisulfite converted counterparts of the subregions of the target markers comprises or is a polynucleotide sequence selected from the group consisting of SEQ ID NOs: 113-166, 168, 169. The SEQ ID NOs of the subregions of each target marker is shown in Table 1 below, and the sequences are provided in FIG. 6.

TABLE 1
Exemplary Subregions of Each Target Marker

| | | Bisulfite | Bisulfite |
|---|---|---|---|
| Target | Genomic | converted | converted |
| Marker | sequence | sequence (C to T) | sequence (G to A) |
| NDRG4 | SEQ ID NO: 86 | SEQ ID NO: 113 | SEQ ID NO: 140 |
| BCAT1 | SEQ ID NO: 87 | SEQ ID NO: 114 | SEQ ID NO: 141 |
| IKZF1 | SEQ ID NO: 88 | SEQ ID NO: 115 | SEQ ID NO: 142 |
| Septin9 | SEQ ID NO: 89 | SEQ ID NO: 116 | SEQ ID NO: 143 |
| SDC2 | SEQ ID NO: 90 | SEQ ID NO: 117 | SEQ ID NO: 144 |
| VAV3 | SEQ ID NO: 91 | SEQ ID NO: 118 | SEQ ID NO: 145 |
| TMEFF2 | SEQ ID NO: 92 | SEQ ID NO: 119 | SEQ ID NO: 146 |
| SALL1 | SEQ ID NO: 93 | SEQ ID NO: 120 | SEQ ID NO: 147 |
| BCAN | SEQ ID NO: 94 | SEQ ID NO: 121 | SEQ ID NO: 148 |
| POU4F2 | SEQ ID NO: 95 | SEQ ID NO: 122 | SEQ ID NO: 149 |
| PKNOX2 | SEQ ID NO: 96 | SEQ ID NO: 123 | SEQ ID NO: 150 |
| ASCL4 | SEQ ID NO: 97 | SEQ ID NO: 124 | SEQ ID NO: 151 |
| KCNA6 | SEQ ID NO: 98 | SEQ ID NO: 125 | SEQ ID NO: 152 |
| SOX1 | SEQ ID NO: 99 | SEQ ID NO: 126 | SEQ ID NO: 153 |
| HS3ST2 | SEQ ID NO: 100 | SEQ ID NO: 127 | SEQ ID NO: 154 |
| FGF12 | SEQ ID NO: 101 | SEQ ID NO: 128 | SEQ ID NO: 155 |
| KCTD8 | SEQ ID NO: 102 | SEQ ID NO: 129 | SEQ ID NO: 156 |
| HMX1 | SEQ ID NO: 103 | SEQ ID NO: 130 | SEQ ID NO: 157 |
| MARCH11 | SEQ ID NO: 104 | SEQ ID NO: 131 | SEQ ID NO: 158 |
| CRHBP | SEQ ID NO: 105 | SEQ ID NO: 132 | SEQ ID NO: 159 |
| NKX2-6 | SEQ ID NO: 106 | SEQ ID NO: 133 | SEQ ID NO: 160 |
| SLC24A2 | SEQ ID NO: 107 | SEQ ID NO: 134 | SEQ ID NO: 161 |
| INTERGENIC | SEQ ID NO: 108 | SEQ ID NO: 135 | SEQ ID NO: 162 |
| REGION 1 | | | |
| INTERGENIC | SEQ ID NO: 109 | SEQ ID NO: 136 | SEQ ID NO: 163 |
| REGION 2 | | | |
| INTERGENIC | SEQ ID NO: 110 | SEQ ID NO: 137 | SEQ ID NO: 164 |
| REGION 3 | | | |
| INTERGENIC | SEQ ID NO: 111 | SEQ ID NO: 138 | SEQ ID NO: 165 |
| REGION 4 | | | |
| INTERGENIC | SEQ ID NO: 112 | SEQ ID NO: 139 | SEQ ID NO: 166 |
| REGION 5 | | | |
| IRF4 | SEQ ID NO: 167 | SEQ ID NO: 168 | SEQ ID NO: 169 |

In certain embodiments, the subregion of NDRG4 comprises a sequence selected from SEQ ID NOs: 86, 113, and 140; the subregion of BCAT1 comprises a sequence selected from SEQ ID NOs: 87, 114, and 141; the subregion of IKZF1 comprises a sequence selected from SEQ ID NOs: 88, 115, and 142: the subregion of Septin9 comprises a sequence selected from SEQ ID NOs: 89, 116, and 143; the subregion of SDC2 comprises a sequence selected from SEQ ID NOs: 90, 117, and 144; the subregion of VAV3 comprises a sequence selected from SEQ ID NOs: 91, 118, and 145; the subregion of TMEFF2 comprises a sequence selected from SEQ ID NOs: 92, 119, and 146; the subregion of SALL1 comprises a sequence selected from SEQ ID NOs: 93, 120, and 147; the subregion of BCAN comprises a sequence selected from SEQ ID NOs: 94, 121, and 148; the subregion of POU4F2 comprises a sequence selected from SEQ ID NOs: 95, 122, and 149; the subregion of PKNOX2 comprises a sequence selected from SEQ ID NOs: 96, 123, and 150; the subregion of ASCL4 comprises a sequence selected from SEQ ID NOs: 97, 124, and 151; the subregion of KCNA6 comprises a sequence selected from SEQ ID NOs: 98, 125, and 152; the subregion of SOX1 comprises a sequence selected from SEQ ID NOs: 99, 126, and 153; the subregion of HS3ST2 comprises a sequence selected from SEQ ID NOs: 100, 127, and 154; the subregion of FGF12 comprises a sequence selected from SEQ ID NOs: 101, 128, and 155; the subregion of KCTD8 comprises a sequence selected from SEQ ID NOs: 102, 129, and 156; the subregion of HMX1 comprises a sequence selected from SEQ ID NOs: 103, 130, and 157; the subregion of MARCH11 comprises a sequence selected from SEQ ID NOs: 104, 131, and 158; the subregion of CRHBP comprises a sequence selected from SEQ ID NOs: 105, 132, and 159; the subregion of NKX2-6 comprises a sequence selected from SEQ ID NOs: 106, 133, and 160; the subregion of SLC24A2 comprises a sequence selected from SEQ ID NOs: 107, 134, and 161; the subregion of INTERGENIC REGION 1 comprises a sequence selected from SEQ ID NOs: 108, 135, and 162; the subregion of INTERGENIC REGION 2 comprises a sequence selected from SEQ ID NOs: 109, 136, and 163; the subregion of INTERGENIC REGION 3 comprises a sequence selected from SEQ ID NOs: 110, 137, and 164; the subregion of INTERGENIC REGION 4 comprises a sequence selected from SEQ ID NOs: 111, 138, and 165; the subregion of INTERGENIC REGION 5 comprises a sequence selected from SEQ ID NOs: 112, 139, and 166; and/or the subregion of IRF4 comprises a sequence selected from SEQ ID NOs: 167, 168, and 169.

In some embodiments, the target marker in the cell-free DNA is present in the biological sample in an amount no more than 1 ng, no more than 0.9 ng, no more than 0.8 ng, no more than 0.7 ng, no more than 0.6 ng, no more than 0.5 ng, no more than 0.4 ng, no more than 0.3 ng, no more than 0.2 ng, no more than 0.1 ng, no more than 0.09 ng, no more than 0.08 ng, no more than 0.07 ng, no more than 0.06 ng, no more than 0.05 ng, no more than 0.04 ng, no more than 0.03 ng, no more than 0.02 ng, or no more than 0.01 ng. In some embodiments, the target marker in the cell-free DNA is present in the biological sample at a percentage of no more than 0.1%, no more than 0.2%, no more than 0.3%, no more than 0.4%, no more than 0.5%, no more than 0.6%, no more than 0.7%, no more than 0.8%, no more than 0.9%, no more than 1%. In some embodiments, the target marker in the cell-free DNA is present in the biological sample at a concentration that is below a level of sensitivity of a detection assay for the target marker. “Sensitivity of a detection assay” is a measure of the detection assay's ability to discriminate between small differences in analyte concentration/amount. If the target marker in the cell-free DNA present in the biological sample is below the level of sensitivity of a detection assay, then it would prevent quantification of the methylation level of each and every of the target markers in the sample using conventional methods. In contrast, the methods disclosed herein are useful and advantageous in detecting very low amount of target markers in the samples. In some embodiments, the target marker in the cell-free DNA is present in the biological sample in an amount of no more than 0.08 ng or no more than 0.04 ng.

In some embodiments, the achieved DNA from step (c) is diluted with a diluent prior to the next step (i.e. step (d)). In some embodiments, the diluent is selected from the group consisting of nuclease free water, Tris-EDTA buffer, and any other buffer which is without PCR inhibition. In some embodiments, the pre-amplified DNA of step (c) is added directly to the next step (i.e. step (d)) without prior dilution.

The at least one target marker within the treated DNA is pre-amplified with a pre-amplification primer pool. As used herein, the term “primer” refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA polymerase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ forward primer that hybridizes with the 5′ end of the sequence to be amplified and a 3′ reverse primer that hybridizes with the complement of the 3′ end of the sequence to be amplified. A person skilled in the art can design primers according to the marker(s) to be amplified based on common knowledge in the art (see, for example, PCR Primer: A Laboratory Manual, Cold Spring Harbor Laboratories, N Y, 1995). Furthermore, several software packages are publicly available for designing optimal probes and/or primers for a variety of assays, e.g. Primer 3 available from the Center for Genome Research, Cambridge, Mass., USA. Clearly, the potential use of the probe or primer should be considered during its design. For example, a primer designed for the purpose of the present invention may include at least one CpG site, or an amplification product obtained from the primer may include at least one CpG site. Tools for designing primers for detecting DNA methylation status are also available in the art, e.g. MethPrimer (Li LC and Dahiya R. MethPrimer: designing primers for methylation PCRs. Bioinformatics. 2002 November; 18(11):1427-31). In the present disclosure, by using the pre-amplification primers as a pool, any target marker(s) (at least a portion of at least one (e.g. each) of the target marker(s) or a subregion of the at least one target marker) within the treated DNA can be pre-amplified.

The term “oligonucleotide” as used herein is defined as a molecule comprising two or more nucleotides (e.g., deoxyribonucleotides or ribonucleotides), preferably at least 5 nucleotides, more preferably at least about 10-15 nucleotides and more preferably at least about 15 to 30 nucleotides, or longer (e.g., oligonucleotides are typically less than 200 residues long (e.g., between 15 and 100 nucleotides), however, as used herein, the term is also intended to encompass longer polynucleotide chains). The exact size will depend on many factors, which in turn depend on the ultimate function or use of the oligonucleotide. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes. Oligonucleotides may be generated in any manner, including chemical synthesis, DNA replication, reverse transcription, PCR, or a combination thereof.

As used herein, the term “complementary” or “complementarity” refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa, Nucleic Acids Res. 12:203 (1984), incorporated herein by reference.

In some embodiments, the pre-amplification primer pool comprises at least one methylation-specific primer pair. In some embodiments, the pre-amplification primer pool comprises multiple methylation-specific primer pairs. In some embodiments, the pre-amplification step is performed by methylation-specific PCR (“MSP”), which is a PCR using methylation-specific primers. This technique (i.e. MSP) has been described in Herman et al., Methylation-specific PCR: a novel PCR assay for methylation status of CpG islands. Proc Natl Acad Sci USA. 1996 Sep. 3; 93 (18): 9821-6, and U.S. Pat. No. 6,265,171.

As used herein, the term “methylation-specific primer pair” refers to a primer pair that is specifically designed to recognize CpG site(s) to take advantage of the differences in methylation to amplify specific target marker(s) within the treated DNA. The primers only act on molecules that with a specific methylation status or without a specific methylation status. For example, the primer may be an oligonucleotide that can specifically hybridize in a methylation-specific manner to a specific CpG site with methylation, but cannot hybridize to the specific CpG site without methylation under stringent conditions, moderately stringent conditions, or highly stringent conditions, and therefore the primer would specifically amplify a target marker that has methylation at the specific CpG site. For another example, the primer may be an oligonucleotide that can specifically hybridize in a methylation-specific manner to a specific CpG site without methylation, but cannot hybridize to the specific CpG site with methylation under stringent conditions, moderately stringent conditions, or highly stringent conditions, and therefore the primer would specifically amplify a target marker that is without methylation at the specific CpG site. Therefore, in the present disclosure, the use of methylation-specific primer pair(s) for the pre-amplification of at least one target marker within the treated DNA allows the differentiation between methylated and unmethylated CpG sites. The methylation-specific primer pair of the present disclosure contains at least one primer which hybridizes to a bisulfite treated CpG dinucleotide. Therefore, the sequence of said primers that are specific for methylated DNA comprises at least one CpG dinucleotide, and the sequence of said primers that are specific for non-methylated DNA contain a “T” at the position of the C position in the CpG, and/or contain a “A” at the position of the G position in the CpG.

In some embodiments, the at least one methylation-specific primer pair comprises a forward primer and a reverse primer each comprising an oligonucleotide sequence that hybridizes under stringent conditions, moderately stringent conditions or highly stringent conditions to at least 9 consecutive nucleotides of one of the target marker(s) (or of the subregion of the target marker(s)), wherein the at least 9 consecutive nucleotides of one of the target marker(s) (or of the subregion of the target marker(s)) comprise at least one (e.g. 1, 2, 3, 4, 5, 6, 7, 8, 9, 10 or more) CpG site.

As used herein, the term “hybridize”, and variations such as “hybridizing”, “hybridizes” or “hybridization” may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the melting temperature (Tm) for the specific sequence at a defined ionic strength and pH.

The Tm can be the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation, Tm=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tm.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% form amide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% form amide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% form amide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, EDTA) contains 3 M sodium chloride, 0.2 M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

In some embodiments, the pre-amplification primer pool further comprises a control primer pair for amplifying a control marker. Usually, a control marker is a nucleic acid having known features (e.g., known sequence, known copy-number per cell), for use in comparison to an experimental target (e.g., a nucleic acid of unknown concentration). A control may be an endogenous, preferably invariant gene against which a test or target nucleic acid in an assay can be normalized. Such normalizing controls for sample-to-sample variations that may occur in, for example, sample processing, assay efficiency, etc., and allows accurate sample-to-sample data comparison, quantifies the amplification efficiency and bias.

In some embodiments, the control marker is selected from the group consisting of ACTB, GAPDH, tubulin, ALDOA, PGK1, LDHA, RPS27A, RPL19, RPL11, ARHGDIA, RPL32, Clorf43, CHMP2A, EMC7, GPI, PSMB2, PSMB4, RAB7A, REEP5, SNRPD3, VCP, and VPS29. In some embodiments, the sequences of control primer pairs are shown in SEQ ID NOs: 55 and 56 in Table 2 below.

In some embodiments, the at least one methylation-specific primer pair comprises one or more pairs of nucleotide sequences selected from the group consisting of SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, and 170/171, as shown in Table 2 below. The sequence numbers of the primer pair(s) used in the present disclosure are expressed in the form of “SEQ ID NOs: n/m”. For example, SEQ ID NOs: 1/2 refer to the primer pair having the nucleic acid sequences as set forth in SEQ ID NO: 1 and SEQ ID NO: 2, respectively, as shown in Table 2 below.

The primer pairs as set forth in SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, and 170/171 are for amplifying the markers NDRG4, BCAT1, IKZF1, Septin9, SDC2, VAV3, TMEFF2, SALL1, BCAN, POU4F2, PKNOX2, INTERGENIC REGION 1, ASCL4, INTERGENIC REGION 2, INTERGENIC REGION 3, KCNA6, SOX1, HS3ST2, FGF12, KCTD8, HMX1, MARCH11, CRHBP, INTERGENIC REGION 4, NKX2-6, SLC24A2, INTERGENIC REGION 5, IRF4, respectively.

In some embodiments, in step (c), the at least one target marker is amplified in the presence of one or more blocker oligonucleotides. The use of such blocker oligonucleotides has been described by Yu et al., BioTechniques 23:714-720, 1997. Blocker sequences are hybridized to the treated DNA concurrently with the pre-amplification primer pair(s). The pre-amplification of the target marker is terminated at the 5′ position of the blocker sequence, such that the pre-amplification of the target marker is suppressed where the complementary sequence to the blocker sequence is present. The blocker sequence may be designed to hybridize to the treated DNA in a methylation status specific manner. For example, for detection of methylated nucleic acids within a population of unmethylated nucleic acids, suppression of the amplification of nucleic acids which are unmethylated at the position in question would be carried out by the use of a blocker sequence comprising a ‘CpA’ or ‘TpA’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivitized at the 3′ position with other than a “free” hydroxyl group. For example, 3′-O-acetyl oligonucleotides arc representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having for example, thiolate bridges at the 5′-terminal thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will no extend the primer toward, and through (in the 5′-3′ direction) the blocker—a process that normally results in degradation of the hybridized blocker oligonucleotide.

A particularly preferred blocker/PCR embodiment, for purposes of the present disclosure and as implemented herein, comprises the use of peptide nucleic acid (PNA) oligomers as blocking oligonucleotides. Such PNA blocker oligomers are ideally suited because they are neither decomposed nor extended by the polymerase.

In certain embodiments, the at least one target marker is/are pre-amplified with a DNA polymerase. As used herein, the term “DNA polymerase” refers to an enzyme that catalyzes the synthesis of polydeoxyribonucleotides from mono-deoxyribonucleoside triphosphates (dNTPs), performing the most fundamental functions of DNA replication, repair, and, in some cases, cell differentiation.

Examples of DNA polymerases in prokaryotes include DNA polymerase I, DNA polymerase II, DNA polymerase III, DNA polymerase IV, and DNA polymerase V. DNA polymerases I, II, and III are known in E. coli. DNA polymerase III appears to be most important in genome replication. DNA polymerase I is important for its ability to edit out unpaired bases at the end of growing strands. Retroviruses possess a unique DNA polymerase, i.e. reverse transcriptase, which uses RNA template to synthesize DNA. As for eukaryotes, examples of DNA polymerases are Polymerases α, β, λ, γ, σ, μ, δ, ε, η, ι, κ, ζ, θ and Rev1. Animal cells have DNA polymerases that are responsible for the replication of DNA in nucleus and mitochondria.

The PCR reagent used in the pre-amplification step may be any commercially available PCR mix (e.g. KAPA2G Fast Multiplex PCR Kit, Luna® Universal Probe qPCR Master Mix, EpiTect MethyLight PCR Kit, etc.) that can be used for amplifying the treated DNA. Alternatively, a person skilled in the art may prepare a PCR reagent including Mg²⁺, dNTP, DNA polymerases, etc. in laboratory. A person skilled in the art may also choose an appropriate PCR reaction system and PCR reaction condition according to their actual need. In some embodiments, the pre-amplification of step (c) comprises from 5 to 30 cycles of reaction, wherein each cycle comprises reaction at 85˜99° C. for 5 seconds to 5 mins before reaction at 40˜80° C. for 5 seconds to 5 mins. In some embodiments, the pre-amplification of step (c) comprises from 10 to 20 cycles of reaction, wherein each cycle comprises reaction at 90˜99° C. for 15 seconds to 2 mins before reaction at 45˜60° C. for 30 seconds to 3 mins. In some embodiments, the pre-amplification of step (c) comprises 15 cycles of reaction, wherein each cycle comprises reaction at 95° C. for 30 seconds before reaction at 56° C. for 60 seconds.

### Step (d)

In step (d) of the methods according to the present disclosure, if step (c) is present, then the methylation level of the at least one target marker is quantified individually based on achieved DNA from step (c); if step (c) is absent, then the methylation level of at least one target marker within the treated DNA obtained from step (b) is quantified individually. In the present disclosure, step (d) may be also designated as a quantification step.

As used herein, the term “methylation state” or “methylation status” refers to the presence, absence and/or quantity of methylation at a particular nucleotide, or nucleotides, within a DNA region. The methylation status of a particular DNA sequence (e.g. target marker as described herein) can indicate the methylation state of every base in the sequence or can indicate the methylation state of a subset of the base pairs (e.g., of cytosine residues or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation level.” A methylation level can be generated, for example, by quantifying the amount of intact DNA present following restriction digestion with a methylation sensitive restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a methylation level, for example from the above described example, represents the methylation status and can thus be used as a quantitative indicator of the methylation status. This is of particular use when it is desirable to compare the methylation status of a sequence in a sample to a threshold level.

Methylation states at one or more particular CpG methylation sites (each having two CpG dinucleotide sequences) within a DNA sequence include “unmethylated,” “fully-methylated” and “hemi-methylated.” The term “hemi-methylation” or “hemimethylation” refers to the methylation state of a double stranded DNA wherein only one strand thereof is methylated. The term “hypermethylation” refers to the average methylation state corresponding to an increased presence of 5-methylcytosine at one or a plurality of CpG dinucleotides within a DNA sequence of a test DNA sample, relative to the amount of 5-methylcytosine found at corresponding CpG dinucleotides within a normal control DNA sample. The methylation status at a residue can be a qualitative or quantitative readout, for example, as indicated by the methylation level. In the present disclosure, the term “methylation status” and “methylation level” may be used interchangeably. According to the present disclosure, it is possible to determine more than one different methylation levels simultaneously.

As described herein, if step (c) is present, then the methylation level of the at least one (e.g. each) target marker is quantified individually based on achieved DNA from step (c); if step (c) is absent, then the methylation level of at least one (e.g. each) target marker within the treated DNA obtained from step (b) is quantified individually, wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one target marker comprises 5 markers selected from the group consisting of Septin9, BCAT1, IKZF1, VAV3, and BCAN. In some embodiments, the at least one target marker comprises 5 markers selected from the group consisting of Septin9, BCAT1, IKZF1, VAV3, and IRF4. In some embodiments, the at least one target marker comprises at least two, three, four, five, six, or seven markers selected from the group consisting of Septin9, BCAT1, IKZF1, NDRG4, BCAN, VAV3, IRF4, or any combination thereof. The detailed description about “target marker” (including but not limited to, the definition of target marker, the specific combination of target markers, etc.) under Section Step (c) above also applies to the “target marker” in “at least one target marker within the treated DNA obtained from step (b)” recited in step (d) (for the scenario where step (c) is absent). The methylation level/status of one or more CpG dinucleotide sequences within a DNA sequence (e.g. a target marker) can be determined by various known assays in the art.

In some embodiments, the quantifying of step (d) is conducted by PCR (e.g. real-time PCR, digital PCR), nucleic acid sequencing, mass-based separation (e.g. electrophoresis, mass spectrometry), or target capture (e.g. hybridization, microarray).

In some embodiments, if step (c) is present, then the methylation level of at least one of the target marker(s) is quantified individually based on the achieved DNA from step (c) by using MSP (see Herman supra). For example, by using one or more primers that hybridize(s) specifically to the unconverted sequence under moderately and/or highly stringent conditions, an amplification product is only produced when a template comprises a methylated cytosine at the CpG site.

In some embodiments, the quantifying of step (d) is conducted by the real-time PCR. Non-limiting examples of the real-time PCR include HeavyMethyl™ PCR described by Cottrell et al., Nucl. Acids Res. 32: e10, 2003; MethyLight™ PCR described by Eads et al., Cancer Res. 59:2302-2306, 1999; Headloop PCR described by Rand et al., Nucl. Acids Res. 33:e 127, 2005.

As used herein, the term “HeavyMethy™ PCR” refers to an art-recognized real-time PCR technique, in which one or more non-extendible nucleic acid (e.g., oligonucleotide) blockers that bind to bisulfite-treated nucleic acid in a methylation specific manner (i.e., the blocker/s bind specifically to unmutated DNA under moderate to high stringency conditions). An amplification reaction is performed using one or more primers that may optionally be methylation specific but that flank the one or more blockers. In the presence of unmethylated nucleic acid (i.e., non-mutated DNA) the blocker/s bind and no PCR product is produced. Using a TaqMann assay essentially as described in, for example, Holland et al., Proc. Natl. Acad. Sci. USA, 88:7276-7280, 1991, the methylation level of nucleic acid in a sample is determined.

As used herein, the term “MethyLight™ PCR” refers to an art-recognized fluorescence-based real-time PCR technique, in which a dual-labelled fluorescent oligonucleotide probe called TaqMan™ probe is employed, and is designed to hybridize to a CpG-rich sequence located between the forward and reverse amplification primers. The TaqMan™ probe comprises a fluorescent “reporter moiety” and a “quencher moiety” covalently bound to linker moieties (e.g., phosphoramidites) attached to the nucleotides of the TaqMan™ oligonucleotide. During PCR amplification, the TaqMan™ probe hybridized to the CpG- reich sequence is cleaved by the 5′ nuclease activity of Taq polymerase, thereby producing detectable signal in a real-time manner during the PCR reaction. In this method, a Molecular Beacon can be used as the detectable probe, and this system is independent of 5′-3′ exonuclease activity of the DNA polymerases used (see Mhlanga and Malmberg, Methods 25:463-471, 2001).

As used herein, the term “Headloop PCR” refers to an art-recognized real-time PCR that selectively amplifies the target nucleic acids, but suppresses amplification of non-amplification target variants by extension of a 3′ stem-loop to form a hairpin structure that can no longer provide a template for further amplification.

In certain embodiments, the real-time PCR is multiplexed real-time PCR.

As used herein, the term “multiplex” or “multiplexed” may refer to an assay or other analytical method in which the presence and/or amount of multiple targets, e.g., multiple nucleic acid sequences, can be assayed simultaneously by using more than one markers, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

In some embodiments, the quantifying of step (d) is conducted by nucleic acid sequencing. Exemplary methods for nucleic acid sequencing are known in the art, see, for example, Frommer et al., Proc. Natl. Acad. Sci. USA 89:1827-1831, 1992; Clark et al., Nucl. Acids Res. 22:2990-2997, 1994. For example, by comparing the sequence obtained using a sample that has not been treated with bisulfite, or the known nucleotide sequence of the region of interest with the sequence obtained using a bisulfite-treated sample facilitates the identification of methylated cytosine(s) in the DNA sequence. Any thymine residue detected at the site of a cytosine in the bisulfite-treated sample compared to an untreated sample may be considered to be caused by mutation as a result of bisulfite treatment, i.e. methylated cytosine is present at this site.

Methods for sequencing DNA are known in the art and include for example, the dideoxy chain termination method or the Maxam-Gilbert method (see Sambrook et al., Molecular Cloning, A Laboratory Manual (2nd Ed., CSHP, New York 1989), pyrosequencing (see Uhlmann et al., Electrophoresis, 23: 4072-4079, 2002), solid phase pyrosequencing (see Landegren et al., Genome Res., 8(8): 769-776, 1998, solid phase minisequencing (see, for example, Southern et al., Genomics, 13:1008-1017, 1992), minisequencing with FRET (see, for example, Chen and Kwok, Nucleic Acids Res. 25:347-353, 1997), sequencing-by-ligation, and ultra-deep sequencing (see Marguiles et al., Nature 437 (7057): 376-80 (2005)).

In certain embodiments, the quantifying of step (d) is conducted by mass-based separation (e.g. electrophoresis, mass spectrometry).

For example, the presence of methylated cytosine residue is detected using combined bisulfite restriction analysis (COBRA) essentially as described in Xiong and Laird, Nucl. Acids Res., 25:2532-2534, 2001. This method exploits the differences in restriction enzyme recognition sites between methylated and unmethylated nucleic acid after treatment with a compound that selectively mutates a non-methylated cytosine residue, e.g., bisulfite. For example, the restriction endonuclease Taql cleaves the sequence TCGA, following bisulfite treatment of a non-methylated nucleic acid the sequence will be TTGA and, as a consequence, will not be cleaved. The digested and/or non-digested nucleic acid is then detected using a detection means known in the art, such as, for example, electrophoresis and/or mass spectrometry.

For another example, different techniques for detecting nucleic acid differences in an amplification product are used based on differences in nucleotide sequence and/or secondary structure after the treatment with a compound that selectively mutates a non-methylated cytosine residue, for example, methylation-specific single stranded conformation analysis (MS-SSCA) (Bianco et al., Hum. Mutat., 14:289-293, 1999), methylation-specific denaturing gradient gel electrophoresis (MS-DGGE) (Abrams and Stanton, Methods Enzymol., 212:71-74, 1992) and methylation-specific denaturing high-performance liquid chromatography (MS-DHPLC) (Deng et al., Chin. J. Cancer Res., 12:171-191, 2000).

In some embodiments, the quantifying of step (d) is conducted by target capture (e.g. hybridization, microarray).

Suitable detection methods by hybridization are known in the art, such as Southern, dot blot, slot blot or other nucleic acid hybridization means (Kawai et al., Mol. Cell. Biol. 14:7421-7427, 1994; Gonzalgo et al., Cancer Res. 57:594-599, 1 97). In some embodiments, the probes for hybridization assay are detectably labeled. In some embodiments, the nucleic acid-based probes for hybridization assay are unlabeled. Such unlabeled probes can be immobilized on a solid support such as a microarray, and can hybridize to the target nucleic acid molecules which are detectably labeled.

An example of microarray is methylation specific microarray, which is useful for differentiating between a sequence with converted cytosine residue(s) and a sequence with unconverted cytosine residue(s) (see Adorjan et al., Nucl. Acids Res., 30: e21, 2002). Hybridization based analysis can also be used for nucleic acids after treatment with a methylation-sensitive restriction enzyme.

For yet another example, the methylation status of the CpG dinucleotide sequences within a DNA sequence may be ascertained by means of oligonucleotide probes that are hybridized to the bisulfite treated DNA concurrently with the PCR amplification primers (wherein said primers may either be methylation specific or standard).

In some embodiments, the step (d) is conducted in the presence of a detection agent. As used herein, the term “detection agent” is an agent used in the quantification step for detecting the presence, absence or amount of nucleic acids.

Various detection agents known in the art can be used in the present disclosure. In some embodiments, the detection agent is selected from the group consisting of a fluorescent probe, an intercalating dye, a chromophore-labeled probe, a radioisotope-labeled probe, and a biotin-labeled probe.

In some embodiments, the fluorescence probe is selected from the group consisting of SEQ ID NOs: 57-85, 172 as shown in Table 2 below.

In some embodiments, the fluorescence probe is labeled with a fluorescent dye (e.g. FAM, HEX/VIC, TAMRA, Texas Red, or Cy5) at its 5′ end, and labeled with a quencher (e.g. BHQ1, BHQ2, BHQ3, DABCYL or TAMRA) at its 3′ end.

Labeling may be done by direct or indirect methods. Direct labeling involves coupling of the label directly (covalently or non-covalently) to the reagent. Indirect labeling involves binding (covalently or non-covalently) of a secondary reagent to the first reagent. The secondary reagent should specifically bind to the first reagent. Said secondary reagent may be coupled with a suitable label and/or be the target (receptor) of tertiary reagent binding to the secondary reagent. The use of secondary, tertiary or even higher order reagents is often to increase the signal intensity. Suitable secondary and higher order reagents may include antibodies, secondary antibodies, and the well-known streptavidin-biotin system (Vector Laboratories, Inc.). The reagent or substrate may also be “tagged” with one or more tags as known in the art.

In some embodiments, if step (c) is present, then the quantifying of step (d) comprises amplifying the achieved DNA from step (c) using quantification primer pair(s) and a DNA polymerase, wherein the at least a portion of the achieved DNA is amplified. In some embodiments, if step (c) is absent, then the quantifying of step (d) comprises amplifying the at least one target marker within the treated DNA obtained from step (b) using quantification primer pair(s) and a DNA polymerase.

As used herein, the term “quantification primer pair(s)” refers to the primer pair(s) that is (are) used in the quantification step.

In some embodiments, if step (c) is present, then the quantification primer pair(s) used in step (d) is (are) capable of hybridizing to at least 9 consecutive nucleotides of the achieved DNA from step (c) under stringent conditions, moderately stringent conditions, or highly stringent conditions. In some embodiments, if step (c) is absent, then the quantification primer pair(s) used in step (d) is (are) capable of hybridizing to at least 9 consecutive nucleotides of the at least one target marker within the treated DNA obtained from step (b) under stringent conditions, moderately stringent conditions, or highly stringent conditions. In some embodiments, if step (c) is present, then at least one (e.g. 1, 2, 3, 4, 5, 6, 7, 8, 9, 10, 11, 12, 13, 14, 15, 16, 17, 18, 19, 20, 21, 22, 23, 24, 25, 26, 27, 28 or more) of the quantification primer pair(s) used in step (d) is identical to at least one (e.g. 1, 2, 3, 4, 5, 6, 7, 8, 9, 10, 11, 12, 13, 14, 15, 16, 17, 18, 19, 20, 21, 22, 23, 24, 25, 26, 27, 28 or more) of the methylation-specific primer pair(s) in the pre-amplification primer pool of step (c).

In some embodiments, if step (c) is absent, then the quantification primer pair(s) used in step (d) is (are) designed to amplify at least a portion within the at least one target marker within the treated DNA obtained from step (b). In some embodiments, if step (c) is present, then the quantification primer pair(s) used in step (d) is (are) designed to amplify at least a portion within the achieved DNA from step (c), i.e. step (c) and step (d) are designed as nested PCR.

Nested PCR is a modification of PCR that was designed to improve sensitivity and specificity. Nested PCR involves the use of two primer sets and two successive PCR reactions. The first round of amplification is conducted to produce a first amplicon, and the second round of amplification is conducted using a primer pair in which one or both of the primers anneal to sites inside the regions defined by the initial primer pair, i.e., the second primer pair is considered to be “nested” within the first primer pair. In this way, background amplification products from the first PCR reaction that do not contain the correct inner sequence are not further amplified in the second PCR reaction.

In some embodiments, if step (c) is present, then the quantifying of step (d) comprises determining the methylation level of at least one (e.g. each) of the target marker(s) based on presence or level of a plurality of CpG dinucleotides, TpG dinucleotides, or CpA dinucleotides in the achieved DNA from step (c). In some embodiments, if step (c) is absent, then the quantifying of step (d) comprises determining the methylation level of at least one (e.g. each) target marker based on presence or level of a plurality of CpG dinucleotides, TpG dinucleotides, or CpA dinucleotides in the at least one target marker within the treated DNA obtained from step (b). In some embodiments, if step (c) is present, then the quantifying of step (d) comprises determining methylation level of cytosine residue(s) based on presence or level of one or more CpG dinucleotides in the achieved DNA from step (c). In some embodiments, if step (c) is absent, then the quantifying of step (d) comprises determining methylation level of cytosine residue(s) based on presence or level of one or more CpG dinucleotides in the at least one target marker within the treated DNA obtained from step (b). In some embodiments, if step (c) is present, then the quantifying of step (d) comprises determining methylation level of cytosine residue(s) based on presence or level of one or more TpG dinucleotides in the achieved DNA from step (c). In some embodiments, if step (c) is absent, then the quantifying of step (d) comprises determining methylation level of cytosine residue(s) based on presence or level of one or more TpG dinucleotides in the at least one target marker within the treated DNA obtained from step (b). In some embodiments, if step (c) is present, then the quantifying of step (d) comprises determining methylation level of cytosine residue(s) based on presence of one or more CpA dinucleotides in the achieved DNA from step (c). In some embodiments, if step (c) is absent, then the quantifying of step (d) comprises determining methylation level of cytosine residue(s) based on presence or level of one or more CpA dinucleotides in the at least one target marker within the treated DNA obtained from step (b).

In some embodiments, if step (c) is present, then the quantification step is performed by partitioning the achieved DNA from step (c) into a plurality of fractions. In some embodiments, if step (c) is absent, then the quantification step is performed by partitioning the at least one target marker within the treated DNA obtained from step (b) into a plurality of fractions. In some embodiments, a plurality of different quantification experiments are conducted with the plurality of fractions, wherein a different set of the achieved DNA from step (c) (or the at least one target marker within the treated DNA obtained from step (b)), if present in the fractions, is quantified in one of the plurality of fractions. In some embodiments, the control marker is quantified in each of the fractions.

### Step (e)

In step (e) of the method of diagnosing colorectal neoplasm, screening for the onset or risk to the onset of colorectal neoplasm or assessing the development or prognosis of colorectal neoplasm in a subject, the methylation level of at least one (e.g. each) target marker from step (d) is compared with a corresponding reference level respectively, wherein an identical or higher methylation level of one or more of the target marker(s) relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In step (e) of the method of monitoring treatment response in a subject who is receiving treatment of colorectal neoplasm, the methylation level of at least one (e.g. each) target marker from step (d) is compared, respectively, with a corresponding methylation level of one or more of the target marker(s) obtained from the same subject prior to the treatment which is quantified by repeating step (a), step (b), optionally step (c), and step (d) with respect to a biological sample containing DNA obtained from the subject prior to the treatment, wherein a lower methylation level of one or more of the target marker(s) relative to its corresponding methylation level prior to the treatment indicates that the subject is responsive to the treatment.

Step (e) of the methods according to the present disclosure may be also designated as a comparison step.

As used herein, the term “compare”, “comparing”, “compared”, or “comparison” refers to comparing the methylation level of at least one (e.g. each) of the target marker(s) from the quantification step comprised by the test biological sample to be analyzed with a corresponding reference level, respectively. It is to be understood that the term as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison may be carried out manually or computer assisted. For a computer assisted comparison, the value of the determined amount may be compared to values corresponding to suitable references which are stored in a database by a computer program. The computer program may further evaluate the result of the comparison, and automatically provide the desired assessment in a suitable output format. Based on the comparison of the methylation level of at least one (e.g. each) of the target marker(s) from the quantification step to a corresponding reference level, it is possible to identify a subject who has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm; it is also possible to monitor treatment response in a subject who is receiving treatment of colorectal neoplasm.

As used herein, the term “reference level” refers to a threshold level which allows for ruling in or ruling out colorectal neoplasm, or the onset or risk to the onset of colorectal neoplasm in a subject, or a threshold level which allows for monitoring treatment response in a subject who is receiving treatment of colorectal neoplasm.

For example, with respect to the method of diagnosing colorectal neoplasm, screening for the onset or risk to the onset of colorectal neoplasm or assessing the development or prognosis of colorectal neoplasm in a subject, if the methylation level of one or more of the target marker(s) in the test sample is identical to or higher than its corresponding reference level, then the subject may be considered as having colorectal neoplasm, or being at the onset or at a risk to the onset of colorectal neoplasm, or developing or being with an increased probability of developing colorectal neoplasm, or having poor prognosis or at a risk to poor prognosis of colorectal neoplasm. In some embodiments, the methylation level of one or more of the target marker(s) in the test sample is at least 1, 2, 3, 4, 5, 6, 7, 8, 9, 10 or more times its corresponding reference level. In the present disclosure, in order to diagnose colorectal neoplasm, screen for the onset or risk to the onset of colorectal neoplasm or assess the development or prognosis of colorectal neoplasm in a subject, it is unnecessary that the methylation level of each and every target marker is identical or higher than its corresponding reference level. Rather, it would be sufficient if the methylation level of at least one target marker quantified in the quantification step is identical or higher than its corresponding reference level.

For another example, with respect to the method of monitoring treatment response in a subject who is receiving treatment of colorectal neoplasm, if the methylation level of one or more of the target marker(s) in the test sample is lower than its corresponding methylation level prior to treatment of colorectal neoplasm, then the subject may be considered as being responsive to the treatment. In some embodiments, the methylation level of one or more of the target marker(s) in the biological sample obtained after treatment of colorectal neoplasm is at least 10%, 20%, 30%, 40%, 50%, 60%, 70%, 80%, 90%, 95%, 99%, 100% lower than its corresponding methylation level prior to treatment of colorectal neoplasm. In the present disclosure, in order to indicate that a subject who is receiving treatment of colorectal neoplasm is responsive to the treatment, it is unnecessary that the methylation level of each and every target marker is lower than its corresponding methylation level prior to treatment of colorectal neoplasm. Rather, it would be sufficient if the methylation level of at least one target marker in the biological sample obtained after treatment of colorectal neoplasm is lower than its corresponding methylation level prior to treatment of colorectal neoplasm.

A reference level of methylation of the target marker may be derived from one or more reference samples, wherein the reference level is obtained from experiments conducted in parallel with the experiment for testing the sample of interest. Alternatively, a reference level may be obtained in a database, which includes a collection of data, standard, or level from one or more reference samples or disease reference samples. In some embodiments, such collection of data, standard or level are normalized so that they can be used for comparison purpose with data from one or more samples. “Normalize” or “normalization” is a process by which a measurement raw data is converted into data that may be directly compared with other so normalized data. Normalization is used to overcome assay-specific errors caused by factors that may vary from one assay to another, for example, variation in loaded quantities, binding efficiency, detection sensitivity, and other various errors.

In some embodiments, a reference database includes methylation levels of the target markers and/or other laboratory and clinical data from one or more reference samples. In some embodiments, a reference database includes methylation levels of the target markers that are each normalized as a percent of the methylation level of a control marker tested under the same conditions as the reference samples. In order to compare with such normalized methylation levels of the target markers, the methylation levels of the target markers of a test sample are also measured and calculated as a percent of the methylation level of a control marker tested under the same conditions as the test sample.

In some embodiments, a reference database is established by compiling reference level data from reference samples obtained from healthy subjects, and/or non-neoplastic subjects (i.e. subjects that are known not to have neoplasm). In some embodiments, a reference database is established by compiling reference level data from reference samples from individuals under treatment for colorectal neoplasm. In some embodiments, a reference database is established by compiling data from reference samples from individuals at different stages of colorectal neoplasm as evidenced by, for example, different methylation levels of the target markers.

A reference level may be chosen by the persons skilled in the art according to the desired sensitivity and specificity. Means for determining suitable reference levels are known to the persons skilled in the art, e. g. a reference level can be determined from data collected from clinical studies.

In some embodiments, the reference levels of step (e) are determined based on the clinical samples obtained from a group of individuals having or at the risk of having colorectal neoplasm and a group of individuals without or are free of the risk of having colorectal neoplasm.

A person skilled in the art can determine whether an individual has or has the risk of having colorectal neoplasm based on various factors, such as age, gender, medical history, family history, symptoms, etc.

In some embodiments, the methylation levels of the target markers and the reference level are expressed as Cycle threshold value (i.e. Ct value). As used herein, the term “Ct value” refers to the cycle number when the fluorescence of a PCR product can be detected above the background signal. Ct values are inversely proportional to the amounts of target markers in the sample, i.e. the lower the Ct value the greater the amount of a target marker in the sample.

For example, in step (e) of the method of diagnosing colorectal neoplasm, screening for the onset or risk to the onset of colorectal neoplasm or assessing the development or prognosis of colorectal neoplasm in a subject, Ct value(s) of the target marker(s) of step (d) is (are) compared with a reference Ct value, wherein an identical or lower Ct value of at least one target marker relative to its corresponding reference Ct value indicates that the subject has colorectal neoplasm, is at the onset or at a risk to the onset of colorectal neoplasm, or develops or with an increased probability of developing colorectal neoplasm, or has poor prognosis or at a risk to poor prognosis of colorectal neoplasm. In some embodiments, if a Ct value of at least one of the multiple target markers of step (d) is lower than its corresponding reference Ct value by 2-10 cycles (for example, 2, 3, 4, 5, 6, 7, 8, 9, 10 cycles), then it is determined that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

As used herein, the term “increased probability” as used herein refers to an overall increase of 5%, 10%, 15%, 20%, 25%, 30%, 40%, 50%, 60%, 70%, 80%, 85%, 90%, 95%, 96%, 97%, 98%, 99% or greater, in the level of likelihood that a subject will develop colorectal neoplasm or poor prognosis of colorectal neoplasm, as compared to a subject from which a reference sample is obtained.

For another example, in step (e) of the method of monitoring treatment response in a subject who is receiving treatment of colorectal neoplasm, Ct value(s) of the target marker(s) of step (d) is (are) compared with a reference Ct value, wherein a higher Ct value of at least one target marker relative to its corresponding Ct value prior to the treatment indicates that the subject who is receiving the treatment of colorectal neoplasm is responsive to the treatment. In some embodiments, if a Ct value of at least one of the multiple target markers of step (d) is higher than its corresponding reference Ct value prior to the treatment by 2-10 cycles (for example, 2, 3, 4, 5, 6, 7, 8, 9, 10 cycles), then it is determined that the subject is responsive to the treatment of colorectal neoplasm.

### Kits

In another aspect, the present disclosure also provides a kit for diagnosing colorectal neoplasm, screening for the onset or risk to the onset of colorectal neoplasm or assessing the development or prognosis of colorectal neoplasm, comprising:
(a) a first reagent for treating a DNA, wherein the first reagent is capable of distinguishing between an unmethylated site and a methylated site in the DNA;
(b) optionally a first primer pool comprising at least one primer pair for pre-amplifying at least one target sequence in at least one target marker selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5, wherein the at least one primer pair is capable of hybridizing under stringent conditions, moderately stringent conditions, or highly stringent conditions to at least 9 consecutive nucleotides of the at least one target sequence treated by the first reagent, and wherein the target sequence comprises at least one CpG site; and
(c) a second reagent, wherein if the first primer pool is present, then the second reagent is for quantifying methylation level of the at least one (e.g. each) target marker pre-amplified by the first primer pool; if the first primer pool is absent, then the second reagent is for quantifying methylation level of at least one (e.g. each) target marker within the DNA treated by the first reagent, wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5.

In some embodiments, the at least one target marker comprises multiple target markers, wherein the multiple target markers comprise at least two (e.g. two, three) markers selected from the group consisting of Septin9, BCAT1, and IKZF1.

In some embodiments, if the first primer pool is present, then the second reagent comprises a second primer pool comprising multiple quantification primer pairs capable of hybridizing under stringent conditions, moderately stringent conditions, or highly stringent conditions to at least 9 consecutive nucleotides of the at least one target sequence pre-amplified by the first primer pool. In some embodiments, if the first primer pool is absent, then the second reagent comprises a third primer pool comprising multiple quantification primer pairs capable of hybridizing under stringent conditions, moderately stringent conditions, or highly stringent conditions to at least 9 consecutive nucleotides of the at least one target sequence of the at least one target marker within the DNA treated by the first reagent.

In some embodiments, if the first primer pool is present, then at least one of the quantification primer pairs in the second primer pool is identical to at least one of the primer pairs in the first primer pool. In some embodiments, if the first primer pool is present, then quantification primer pairs of the second primer pool are designed to amplify at least a portion within the at least one target sequence pre-amplified by the first primer pool. In some embodiments, if the first primer pool is absent, then quantification primer pairs of the third primer pool are designed to amplify at least a portion within the at least one target sequence of the at least one target marker within the DNA treated by the first reagent. In some embodiments, the first, second, or third primer pool comprises at least one methylation-specific primer pair.

In some embodiments, the first primer pool and the second primer pool are packaged in a single container or in separate containers. In some embodiments, the kit further comprises one or more blocker oligonucleotides.

In some embodiments, the kit further comprises a detection agent. In some embodiments, the detection agent is selected from the group consisting of a fluorescent probe, an intercalating dye, a chromophore-labeled probe, a radioisotope-labeled probe, and a biotin-labeled probe. In some embodiments, the fluorescent probe comprises an oligonucleotide sequence selected from the group consisting of SEQ ID NOs: 57-85, 172. In some embodiments, the fluorescent probe is labeled with a fluorescent dye (e.g. FAM, HEX/VIC, TAMRA, Texas Red, or Cy5) at its 5′ end, and labeled with a quencher (e.g. BHQ1, BHQ2, BHQ3, DABCYL, TAMRA or lowa Black Dark Quenchers) at its 3′ end.

In some embodiments, the kit further comprises a DNA polymerase and/or a container suitable for containing the biological sample from the subject. In some embodiments, the kit further comprises an instruction for use and/or interpretation of the kit results.

In some embodiments, the kit may contain, packaged in separate containers, a reaction buffer optimized for primer extension mediated by the polymerase, such as PCR. Preferred is a kit, which further comprises a container suitable for containing the means for determining methylation of at least one (e.g. 1, 2, 3, 4, 5, 6, 7, 8, 9, 10, 11, 12, 13, 14, 15, 16, 17, 18, 19, 20, 21, 22, 23, 24, 25, 26, 27, 28 or more) target marker selected from the group consisting of Septin9, BCAT1, IKZF1, BCAN, VAV3, IRF4, POU4F2, SALL1, PKNOX2, SDC2, ASCL4, INTERGENIC REGION 1, TMEFF2, INTERGENIC REGION 4, NKX2-6, INTERGENIC REGION 5, SLC24A2, NDRG4, INTERGENIC REGION 2, INTERGENIC REGION 3, KCNA6, SOX1, HS3ST2, FGF12, KCTD8, HMX1, MARCH11, and CRHBP in the biological sample of the subject.

In some embodiments, the first reagent comprises a bisulfite reagent or methylation sensitive restriction enzyme (MSRE). In some embodiments, the bisulfite reagent is selected from the group consisting of ammonium bisulfite, sodium bisulfite, potassium bisulfite, calcium bisulfite, magnesium bisulfite, aluminum bisulfite, hydrogen sulfite and any combination thereof. In some embodiments, the bisulfite reagent is sodium bisulfite. In some embodiments, the MSRE is selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI and any combination thereof.

In some embodiments, the first primer pool comprises at least one methylation-specific primer pair for pre-amplifying at least one target sequence in at least one target marker selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5.

In some embodiments, the at least one target marker comprises one or multiple markers (e.g. at least 2, 3, 4, 5, 6, 7, 8, 9, 10, 11, 12, 13, 14, 15, 16, 17, 18, 19, 20, 21, 22, 23, 24, 25, 26, 27, 28 markers) selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5.

In some embodiments, the at least one target marker can be up to one target marker (i.e. one marker but no more than one marker). In some embodiments, the at least one target marker is Septin9. In some embodiments, the at least one target marker is BCAT1. In some embodiments, the at least one target marker is IKZF1.

In some embodiments, the at least one target marker is NDRG4. In some embodiments, the at least one target marker is BCAN. In some embodiments, the at least one target marker is PKNOX2. In some embodiments, the at least one target marker is VAV3. In some embodiments, the at least one target marker is IRF4. In some embodiments, the at least one target marker is POU4F2. In some embodiments, the at least one target marker is SALL1. In some embodiments, the at least one target marker is TMEFF2. In some embodiments, the at least one target marker is ASCL4. In some embodiments, the at least one target marker is FGF12. In some embodiments, the at least one target marker is INTERGENIC REGION 1.

In some embodiments, the at least one target marker comprises multiple target markers. In some embodiments, the multiple target markers comprise at least two or three markers selected from the group consisting of Septin9, BCAT1, and IKZF1. In some embodiments, the multiple target markers of the present disclosure further comprise one, two, three, four, or five additional markers selected from the group consisting of BCAN, PKNOX2, VAV3, NDRG4 and IRF4. In some embodiments, the multiple target markers of the present disclosure further comprise one or more (e.g. 1, 2, 3, 4, 5, 6, 7, 8, 9, 10, 11, 12, 13, 14, 15, 16, 17, 18, 19, 20) additional markers selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP.

In some embodiments, the multiple target markers of the present disclosure comprise Septin9 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, BCAT1, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, BCAT1, IKZF1, NDRG4, PKNOX2, VAV3, IRF4, or any combination thereof. In some embodiments, the at least one (e.g. at least 1, 2, or 3) additional target marker comprises BCAN, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise BCAT1 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, Septin9, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, Septin9, NDRG4, IKZF1, PKNOX2, VAV3, IRF4, or any combination thereof. In some embodiments, the at least one (e.g. at least 1, 2, or 3) additional target marker comprises NDRG4, Septin9, and/or IKZF1. In some embodiments, the at least one (e.g. at least 1, 2, or 3) additional target marker comprises BCAN, VAV3, and/or IRF4.

In some embodiments, the multiple target markers of the present disclosure comprise IKZF1 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, Septin9, BCAT1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, Septin9, BCAT1, PKNOX2, NDRG4, VAV3, IRF4, or any combination thereof. In some embodiments, the at least one (e.g. at least 1, 2, or 3) additional target marker comprises NDRG4, Septin9, and/or BCAT1. In some embodiments, the at least one (e.g. at least 1, 2, or 3) additional target marker comprises BCAN, VAV3, and/or IRF4.

In some embodiments, the multiple target markers of the present disclosure comprise BCAN and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, NDRG4, IRF4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises NDRG4, VAV3, and/or IRF4.

In some embodiments, the multiple target markers of the present disclosure comprise VAV3 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, BCAN, PKNOX2, NDRG4, IRF4 or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, NDRG4, and/or IRF4.

In some embodiments, the multiple target markers of the present disclosure comprise IRF4 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, VAV3, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, BCAN, NDRG4, PKNOX2, VAV3 or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or NDRG4.

In some embodiments, the multiple target markers of the present disclosure comprise PKNOX2 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, VAV3, IRF4, POU4F2, SALL1, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, BCAN, VAV3, NDRG4, IRF4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise NDRG4 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, BCAN, POU4F2, PKNOX2, SDC2, TMEFF2, SALL1, SLC24A2, NKX2-6, KCNA6, SOX1, HS3ST2, ASCL4, KCTD8, HMX1, MARCH11, CRHBP, FGF12, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, IRF4, BCAN, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the respective target marker comprises or is: a) the respective region defined by Hg19 coordinates as set forth below:

| Target Marker | Hg19 Coordinate |
|---|---|
| NDRG4 | chr16: 58496750-58547532 |
| BCAT1 | chr12: 24964295-25102393 |
| IKZF1 | chr7: 50343720-50472799 |
| Septin9 | chr17: 75276651-75496678 |
| SDC2 | chr8: 97505579-97624000 |
| VAV3 | chr1: 108113782-108507766 |
| IRF4 | chr6: 391739-411447 |
| TMEFF2 | chr2: 192813769-193060435 |
| SALL1 | chr16: 51169886-51185278 |
| BCAN | chr1: 156611182-156629324 |
| POU4F2 | chr4: 147560045-147563626 |
| PKNOX2 | chr11: 125034583-125303285 |
| ASCL4 | chr12: 108168162-108170421 |
| KCNA6 | chr12: 4918342-4960277 |
| SOX1 | chr13: 112721913-112726020 |
| HS3ST2 | chr16: 22825498-22927659 |
| FGF12 | chr3: 191857184-192485553 |
| KCTD8 | chr4: 44175926-44450824 |
| HMX1 | chr4: 8847802-8873543 |
| MARCH11 | chr5: 16067248-16180871 |
| CRHBP | chr5: 76248538-76276983 |
| NKX2-6 | chr8: 23559964-23564111 |
| SLC24A2 | chr9: 19507450-19786926 |
| INTERGENIC REGION 1 | chr6: 19679885-19693988 |
| INTERGENIC REGION 2 | chr10: 130082033-130087148 |
| INTERGENIC REGION 3 | chr10: 133107880-133113966 |
| INTERGENIC REGION 4 | chr7: 152620588-152624685 |
| INTERGENIC REGION 5 | chr8: 70945014-70949177, |

and 5 kb upstream of the respective start site and 5 kb downstream of the respective end site of each region described above, or b)a bisulfite converted counterpart of a), or c) a MSRE treated counterpart of a).

In some embodiments, if the first primer pool is present, then the first primer pool comprises at least one primer pair comprising or consisting of at least one pair of nucleotide sequences selected from the group consisting of SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, and 170/171 as shown in Table 2 below, and optionally wherein the second primer pool comprises at least one primer pair that is identical to at least one of the primer pairs in the first primer pool. In some embodiments, if the first primer pool is absent, then the third primer pool comprises at least one primer pair comprising or consisting of at least one pair of nucleotide sequences selected from the group consisting of SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, and 170/171 as shown in Table 2 below.

In some embodiments, the first primer pool, the second primer pool, or optionally the third primer pool further comprises a primer pair for amplifying a control marker. In some embodiments, the control marker is selected from the group consisting of ACTB, GAPDH, tubulin, ALDOA, PGK1, LDHA, RPS27A, RPL19, RPL11, ARHGDIA, RPL32, Clorf43, CHMP2A, EMC7, GPI, PSMB2, PSMB4, RAB7A, REEP5, SNRPD3, VCP, and VPS29.

In some embodiments, the kit further comprises a plurality of containers, each for receiving a fraction of the second primer pool.

In some embodiments, the kit further comprises standard reagents for performing a CpG position-specific methylation analysis, wherein said analysis comprises one or more of the following techniques: MS-SNuPE, MSP, MethyLight™, HeavyMethyl™, COBRA, and nucleic acid sequencing.

In some embodiments, the kit may comprise additional reagents selected from the group consisting of buffer (e.g., restriction enzyme, PCR, storage or washing buffers); DNA recovery reagents or kits (e.g., precipitation, ultrafiltration, affinity column) and DNA recovery components.

In some embodiments, the kit of the present disclosure may comprise:
(a) a bisulfite reagent;
(b) optionally a first primer pool comprising multiple methylation-specific primer pairs for pre-amplifying at least two target sequences in multiple target markers comprising at least two (e.g. 2, 3, 4, 5, 6, 7, 8, 9, 10, 11, 12, 13, 14, 15, 16, 17, 18, 19, 20, 21, 22, 23, 24, 25, 26, 27, 28 or more) markers selected from the group consisting of Septin9, BCAT1, IKZF1, BCAN, VAV3, IRF4, POU4F2, SALL1, PKNOX2, SDC2, ASCL4, INTERGENIC REGION 1, TMEFF2, INTERGENIC REGION 4, NKX2-6, INTERGENIC REGION 5, SLC24A2, NDRG4, INTERGENIC REGION 2, INTERGENIC REGION 3, KCNA6, SOX1, HS3ST2, FGF12, KCTD8, HMX1, MARCH11, and CRHBP, wherein the methylation-specific primer pairs comprise or consist of at least two pairs of nucleotide sequences selected from the group consisting of SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, and 170/171 as shown in Table 2 below;
(c) a second reagent, wherein if the first primer pool is present, then the second reagent is for quantifying methylation level of at least one (e.g. each) of the multiple target markers pre-amplified by the first primer pool, wherein the second reagent comprises a second primer pool comprising multiple quantification primer pairs capable of hybridizing under stringent conditions, moderately stringent conditions, or highly stringent conditions to at least 9 consecutive nucleotides of the multiple target markers pre-amplified by the first primer pool; if the first primer pool is absent, then the second reagent is for quantifying methylation level of at least one (e.g. each) target marker within the DNA treated by the first reagent, wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5, wherein the second reagent comprises a third primer pool comprising multiple quantification primer pairs capable of hybridizing under stringent conditions, moderately stringent conditions, or highly stringent conditions to at least 9 consecutive nucleotides of the at least one target sequence of the at least one target marker within the DNA treated by the first reagent.

The kit of the present disclosure may also contain other components such as buffers or solutions suitable for blocking, washing or coating, packaged in a separate container.

The kit of the present disclosure may further comprise one or several of the following components, which are known in the art for DNA enrichment: a protein component, said protein binding selectively to methylated DNA; a triplex-forming nucleic acid component, one or a plurality of linkers, optionally in a suitable solution; substances or solutions for performing a ligation e.g. ligases, buffers; substances or solutions for performing a column chromatography; substances or solutions for performing an immunology based enrichment (e.g. immunoprecipitation); substances or solutions for performing a nucleic acid amplification e.g. PCR; a dye or several dyes, if applicable with a coupling reagent, if applicable in a solution; substances or solutions for performing a hybridization; and/or substances or solutions for performing a washing step.

### Uses

In another aspect, the present disclosure provides use of the kit of the present disclosure in the manufacture of a diagnostic kit for diagnosing colorectal neoplasm, screening for the onset or risk to the onset of colorectal neoplasm, or assessing the development or prognosis of colorectal neoplasm in the subject, or monitoring treatment response in a subject who is receiving treatment of colorectal neoplasm.

In another aspect, the present disclosure provides use of a reagent for quantifying methylation level of a target marker in the manufacture of a kit for using in a method of diagnosing colorectal neoplasm, screening for the onset or risk to the onset of colorectal neoplasm, or assessing the development or prognosis of colorectal neoplasm in a subject, wherein said method comprising the following steps:
(a) obtaining a biological sample containing DNA from the subject;
(b) treating the DNA in the biological sample obtained from step (a) with a reagent capable of distinguishing between unmethylated and methylated CpG site(s) in the DNA, thereby obtaining a treated DNA;
(c) pre-amplifying at least a portion of at least one target marker within the treated DNA obtained from step (b) with a pre-amplification primer pool, wherein at least a portion of at least one (e.g. each) of the target marker(s) is pre-amplified, and the at least one target marker comprise one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d) if step (c) is present, then quantifying individually methylation level of the at least one (e.g. each) target marker based on achieved DNA from step (c); if step (c) is absent, then quantifying individually methylation level of at least one (e.g. each) target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(e) comparing the methylation level of at least one (e.g. each) target marker from step (d) respectively with a corresponding reference level, wherein an identical or higher methylation level of one or more of the target marker(s) relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

In another aspect, the present disclosure provides use of a reagent for quantifying methylation level of a target marker in the manufacture of a kit for using in a method of monitoring treatment response in a subject who is receiving treatment of colorectal neoplasm, wherein said method comprising the following steps:
(a) obtaining a biological sample containing DNA from the subject;
(b) treating the DNA in the biological sample obtained from step (a) with a reagent capable of distinguishing between unmethylated and methylated CpG site(s) in the DNA, thereby obtaining a treated DNA;
(c) pre-amplifying at least a portion of at least one target marker within the treated DNA obtained from step (b) with a pre-amplification primer pool, wherein at least a portion of at least one (e.g. each) of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d) if step (c) is present, then quantifying individually methylation level of the at least one (e.g. each) target marker based on achieved DNA from step (c); if step (c) is absent, then quantifying individually methylation level of at least one (e.g. each) target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(e) comparing the methylation level of at least one (e.g. each) target marker from step (d) respectively with a corresponding methylation level of one or more of the target marker(s) obtained from the same subject prior to the treatment which is quantified by repeating step (a), step (b), optionally step (c), and step (d) with respect to a biological sample containing DNA obtained from the subject prior to the treatment, wherein a lower methylation level of one or more of the target marker(s) relative to its corresponding methylation level prior to the treatment indicates that the subject is responsive to the treatment.

In some embodiments, the at least one target marker of step (c) above comprises one or multiple markers (e.g. at least 2, 3, 4, 5, 6, 7, 8, 9, 10, 11, 12, 13, 14, 15, 16, 17, 18, 19, 20, 21, 22, 23, 24, 25, 26, 27, 28 markers) selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5.

In some embodiments, the at least one target marker of step (c) above can be up to one target marker (i.e. one marker but no more than one marker). In some embodiments, the at least one target marker is Septin9. In some embodiments, the at least one target marker is BCAT1. In some embodiments, the at least one target marker is IKZF1. In some embodiments, the at least one target marker is BCAN. In some embodiments, the at least one target marker is PKNOX2. In some embodiments, the at least one target marker is VAV3. In some embodiments, the at least one target marker is IRF4. In some embodiments, the at least one target marker is NDRG4. In some embodiments, the at least one target marker is POU4F2. In some embodiments, the at least one target marker is SALL1. In some embodiments, the at least one target marker is TMEFF2. In some embodiments, the at least one target marker is ASCL4. In some embodiments, the at least one target marker is FGF12. In some embodiments, the at least one target marker is INTERGENIC REGION 1.

In some embodiments, the at least one target marker of step (c) above comprises multiple target markers. In some embodiments, the multiple target markers comprise at least two or three markers selected from the group consisting of Septin9, BCAT1, and IKZF1. In some embodiments, the multiple target markers of the present disclosure further comprise one two, three, four, or five additional markers selected from the group consisting of BCAN, PKNOX2, VAV3, NDRG4 and IRF4. In some embodiments, the multiple target markers of the present disclosure further comprise one or more (e.g. 1, 2, 3, 4, 5, 6, 7, 8, 9, 10, 11, 12, 13, 14, 15, 16, 17, 18, 19, 20) additional markers selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP.

In some embodiments, the multiple target markers of the present disclosure comprise Septin9 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, BCAT1, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, BCAT1, IKZF1, NDRG4, PKNOX2, VAV3, IRF4, or any combination thereof. In some embodiments, the at least one (e.g. at least 1, 2, or 3) additional target marker comprises BCAN, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise BCAT1 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, Septin9, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, Septin9, NDRG4, IKZF1, PKNOX2, VAV3, IRF4, or any combination thereof. In some embodiments, the at least one (e.g. at least 1, 2, or 3) additional target marker comprises NDRG4, Septin9, and/or IKZF1. In some embodiments, the at least one (e.g. at least 1, 2, or 3) additional target marker comprises BCAN, VAV3, and/or IRF4.

In some embodiments, the multiple target markers of the present disclosure comprise IKZF1 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of BCAN, Septin9, BCAT1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises BCAN, Septin9, BCAT1, PKNOX2, NDRG4, VAV3, IRF4, or any combination thereof. In some embodiments, the at least one (e.g. at least 1, 2, or 3) additional target marker comprises NDRG4, Septin9, and/or BCAT1. In some embodiments, the at least one (e.g. at least 1, 2, or 3) additional target marker comprises BCAN, VAV3, and/or IRF4.

In some embodiments, the multiple target markers of the present disclosure comprise BCAN and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, VAV3, NDRG4, IRF4, PKNOX2, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise VAV3 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, BCAN, PKNOX2, NDRG4, IRF4, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise IRF4 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, VAV3, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprise Septin9, BCAT1, IKZF1, BCAN, NDRG4, PKNOX2, VAV3 or any combination thereof. In some embodiments, the at least one (e.g. at least 1, 2, or 3) additional target marker comprise Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise PKNOX2 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, BCAN, VAV3, IRF4, POU4F2, SALL1, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprise Septin9, BCAT1, IKZF1, BCAN, VAV3, NDRG4, IRF4, or any combination thereof. In some embodiments, the at least one (e.g. at least 1, 2, or 3) additional target marker comprise Septin9, BCAT1, and/or IKZF1. In some embodiments, the at least one (e.g. at least 1, 2, or 3) additional target marker comprises BCAN, VAV3, and/or IRF4.

In some embodiments, the multiple target markers of the present disclosure comprise NDRG4 and at least one (e.g. at least 1, 2, 3, 4, 5, 6, 7, 8, 9, 10, 11, 12, 13, 14, 15, 16, 17, 18, 19, 20, 21, 22, 23, 24, 25, 26, or 27) additional target marker selected from the group consisting of Septin9, BCAT1, IKZF1, VAV3, IRF4, BCAN, POU4F2, PKNOX2, SDC2, TMEFF2, SALL1, SLC24A2, NKX2-6, KCNA6, SOX1, HS3ST2, ASCL4, KCTD8, HMX1, MARCH11, CRHBP, FGF12, INTERGENIC REGION 1, INTERGENIC REGION 2, INTERGENIC REGION 3, INTERGENIC REGION 4, and INTERGENIC REGION 5. In some embodiments, the at least one (e.g. at least 1, 2, 3, 4, 5, 6, or 7) additional target marker comprises Septin9, BCAT1, IKZF1, PKNOX2, VAV3, IRF4, BCAN, or any combination thereof. In some embodiments, the at least one (e.g. at least 1, 2, or 3) additional target marker comprises Septin9, BCAT1, and/or IKZF1. In some embodiments, the at least one (e.g. at least 1, 2, or 3) additional target marker comprises BCAN, VAV3, and/or IRF4.

### Embodiments

The biological materials used in all examples, various clones and expression plasmids, media, enzymes, buffer solutions, and various culturing methods, protein extraction and purification methods, and the other molecular biological operation methods, are all well-known to those of skill in the art. For more details, please refer to the “Molecular Cloning: A Laboratory Manual” edited by Sambrook, et al. (Cold Spring Harbor, 1989) and “Short Protocols in Molecular Biology” (Frederick M. Ausubel, et al., translated by Yan Ziying et al., Science Press (Beijing), 1998).

### Example 1: Verification of Methylation-Specific Primers

For the initial proof-of-concept, the inventors selected bisulfite-converted reference DNA to assess primer/probe specificity. Customized primer/probe sets were designed for 28 target markers (i.e. NDRG4, BCAT1, IKZF1, Septin9, SDC2, VAV3, IRF4, TMEFF2, SALL1, BCAN, POU4F2, PKNOX2, ASCL4, KCNA6, SOX1, HS3ST2, FGF12, KCTD8, HMX1, MARCH11, CRHBP, NKX2-6, SLC24A2 and 5 intergenic regions, including INTERGENIC REGION 1, INTERGENIC REGION 2, INTERGENIC REGION 3, INTERGENIC REGION 4, and INTERGENIC REGION 5). In a proof-of-concept experiment, the inventors created mixtures (10%, 25%, 50%, 100%) of fully methylated DNA at all CpG sites into fully unmethylated DNA with 4 ng as the total input. 28 target markers were evaluated on these mixtures in triplicates, using primers, and probes having sequences shown in Table 2. The experimental methods are detailed below.

Bisulfite converted fully methylated DNA and bisulfite converted fully unmethylated DNA were purchased from Qiagen company (EpiTect Control DNA), and were mixed to provide for mixed DNA compositions containing 100%, 50%, 25%, and 10% of fully methylated DNA in the fully unmethylated DNA, respectively, where the total amount of DNA was 4 ng in each mixed DNA composition.

The mixed DNA compositions were amplified by PCR reactions in the presence of methylation-specific primer pairs (see Table 2) and detection probes (see Table 2) specific for 28 target markers (i.e. NDRG4, BCAT1, IKZF1, Septin9, SDC2, VAV3, IRF4, TMEFF2, SALL1, BCAN, POU4F2, PKNOX2, ASCL4, KCNA6, SOX1, HS3ST2, FGF12, KCTD8, HMX1, MARCH11, CRHBP, NKX2-6, SLC24A2 and 5 intergenic regions, including INTERGENIC REGION 1, INTERGENIC REGION 2, INTERGENIC REGION 3, INTERGENIC REGION 4, and INTERGENIC REGION 5). Control marker ACTB was also amplified in the PCR reaction with methylation-non-specific primers (see Table 2), and detection probe (see Table 2). Each of the 28 target markers and one control marker was amplified respectively in separate detection assays. The detection probes for different markers were labeled with different fluorescence (FAM, HEX, VIC, TAMRA, Texas Red, or Cy5) and corresponding quenchers (BHQ1, BHQ2, BHQ3, DABCYL or TAMRA). In the PCR reaction system, each of the primers was at a final concentration of 500 nM, and each of the detection probes was at a final concentration of 200 nM.

TABLE 2
The sequences of primer pairs and probes for each target marker.

| Marker | F primer sequence | R primer sequence | Probe Sequence |
|---|---|---|---|
| NDRG4 | CAACGCACCCAACACA | GCGGAGTTTGGGGGA | GTCGATTCGCGTTTTCGTCG |
| | (SEQ ID NO: 1) | (SEQ ID NO: 2) | (SEQ ID NO: 57) |
| BCAT1 | TACGTGGCGGGTTGG | AAAAAAACAACCTTAATATCTTC | TCGGTTTTTTCGCGGCG |
| | (SEQ ID NO: 3) | (SEQ ID NO: 4) | (SEQ ID NO: 58) |
| IKZF1 | GTTTTTTTGGTTCGGAGTTG | CAAAACGAAACACGAAAAAAATA | CGCCCCGTCGCCGAAT |
| | (SEQ ID NO: 5) | (SEQ ID NO: 6) | (SEQ ID NO: 59) |
| Septin9 | GTAGTTGGATGGGATTATTT | CACCCGCAAAATCCTCT | TTGTTGCGGTCGCGGACG |
| | (SEQ ID NO: 7) | (SEQ ID NO: 8) | (SEQ ID NO: 60) |
| SDC2 | GGAGTGTAGAAATTAATAAG | CTCGCTTCCTCCTCCTAC | AGGGCGTCGCGTTTTCGGG |
| | (SEQ ID NO: 9) | (SEQ ID NO: 10) | (SEQ ID NO: 61) |
| VAV3 | CGGAGTCGAGTTTAG | ACCGCCGACCCTTT | TTTCGATTTCGCGCGGGG |
| | (SEQ ID NO: 11) | (SEQ ID NO: 12) | (SEQ ID NO: 62) |
| TMEFF2 | GTAATATTTAGGGATTGGG | CTCCTTATAACAACAACTTC | TGCGCCGGAGACGCG |
| | (SEQ ID NO: 13) | (SEQ ID NO: 14) | (SEQ ID NO: 63) |
| SALL1 | GAGGGTGGGTTTGGTAA | GATATAAAAACAACCCTCCA | CGCGTTCGAGTTAAGAGTCGCG |
| | (SEQ ID NO: 15) | (SEQ ID NO: 16) | (SEQ ID NO: 64) |
| BCAN | GGGAAGAAAGGGGGTTTTGT | TACGACGAAAACTACGCGAA | CGTCGGGAGGGTCGG |
| | (SEQ ID NO: 17) | (SEQ ID NO: 18) | (SEQ ID NO: 65) |
| POU4F2 | AACATCCGTTCAAACTAACA | GGTTGTGCGAAGTTGAG | CGTCGTCGTTTTCGGATTTTGTACG |
| | (SEQ ID NO: 19) | (SEQ ID NO: 20) | (SEQ ID NO: 66) |
| PKNOX2 | GTTTTAGGAGTTATTTGGGTTTGC | ACTATAACACCTCGCTACTAACGCT | CGGTGGTTCGTAGGGGTCGCG |
| | (SEQ ID NO: 21) | (SEQ ID NO: 22) | (SEQ ID NO: 67) |
| | | | CGTAGCGCGGCCGGG |
| | | | (SEQ ID NO: 68) |
| INTERGENIC | TTTTTGAAAGTTTGAGAAAATGT | CCGACGCCTCTACCAA | TTCGTTATTTGGGTCGCGGG |
| REGION 1 | (SEQ ID NO: 23) | (SEQ ID NO: 24) | (SEQ ID NO: 69) |
| ASCL4 | TTGTTGGAGYGTTAGGTTTGG | CCRAAAAAACCTTAAACTCCCC | CGACGCCGACCGCGCCCTCG |
| | (SEQ ID NO: 25) | (SEQ ID NO: 26) | (SEQ ID NO: 70) |
| INTERGENIC | TTATTTCGGGGAAGGTTACG | GCGAAAACGAAATCATAAAATAAAC | TCGGACGCGTTTTCGGG |
| REGION 2 | (SEQ ID NO: 27) | (SEQ ID NO: 28) | (SEQ ID NO: 71) |
| INTERGENIC | CGAGTCGAGTTTGGGT | ACCTCCGAAACAAAATCTA | CGCGTAGTTATCGTTAGACGGCG |
| REGION 3 | (SEQ ID NO: 29) | (SEQ ID NO: 30) | (SEQ ID NO: 72) |
| KCNA6 | TGTTAGAGTTTATTGGGATG | GAAAACCGAATCTCAAACAC | TCGAAAAGACGCGTGGTTTCGT |
| | (SEQ ID NO: 31) | (SEQ ID NO: 32) | (SEQ ID NO: 73) |
| SOX1 | ATACGGGAGAAAGAGTACGTTA | AACGTAACCGTACAACCTAAACG | GGTTACGCGGCGCGTGG |
| | (SEQ ID NO: 33) | (SEQ ID NO: 34) | (SEQ ID NO: 74) |
| HS3ST2 | TAGTTTTCGGAGAAGACGGC | CTATAACCCTACGATCGCCT | TCGTGGTAGCGTTACGCGA |
| | (SEQ ID NO: 35) | (SEQ ID NO: 36) | (SEQ ID NO: 75) |
| FGF12 | AGGGAGTTTAATAGCGATCGAGT | TTTACTAAACACCCCGAAAAC | AGACGGGCGTTTTTTGTGCGA |
| | (SEQ ID NO: 37) | (SEQ ID NO: 38) | (SEQ ID NO: 76) |
| KCTD8 | AGGTCGGTTTTTATATGGTG | TCGATATAACTACTCCAAATC | TCGTTAATTAGTATCGCGACGA |
| | (SEQ ID NO: 39) | (SEQ ID NO: 40) | (SEQ ID NO: 77) |
| HMX1 | GGGAGGGGGTAGTAGG | CGCTCATTTAATTTAAATTTATTTC | AGTCGGTCGAGGTTTTCGT |
| | (SEQ ID NO: 41) | (SEQ ID NO: 42) | (SEQ ID NO: 78) |
| MARCH11 | GGGCGCGATAGTTTGAG | CCCGCGCCCTTTCC | TGTTTTGGGCGCGTTCGA |
| | (SEQ ID NO: 43) | (SEQ ID NO: 44) | (SEQ ID NO: 79) |
| CRHBP | GGGGCGCGGTTTTTTTA | CTAAACTACGCTAAATTCCT | CGCGTTCGGGCGT |
| | (SEQ ID NO: 45) | (SEQ ID NO: 46) | (SEQ ID NO: 80) |
| INTERGENIC | AGGGATTTAGGTTAGGGGTC | ACGACATCCTTCAAACCGAC | TTCGTTTCGGGGCGGGG |
| REGION 4 | (SEQ ID NO: 47) | (SEQ ID NO: 48) | (SEQ ID NO: 81) |
| NKX2-6 | AGGTTCGGGTGAGGAG | AAACGTCTATCCCAAAACTT | CGTTTTGTCGTTGTAGGTTTCGT |
| | (SEQ ID NO: 49) | (SEQ ID NO: 50) | (SEQ ID NO: 82) |
| SLC24A2 | AGTTAAAAGTAAGGGTAGGA | CCCCGCTAAAAATTAACCA | CGGGGGTTTTAAATTTACGTTTCG |
| | (SEQ ID NO: 51) | (SEQ ID NO: 52) | (SEQ ID NO: 83) |
| INTERGENIC | GGTCGGGTTGAGATTGG | GGTGGGGTTGAGATTGG | CGCTTTTTGTCGGGGTGCGG |
| REGION 5 | (SEQ ID NO: 53) | (SEQ ID NO: 54) | (SEQ ID NO: 84) |
| IRF4 | AAAAAAAAAAAAACTCCACATTT | TAGTTGNGGAGTTTGGG | ATCGTACGTAAGGTTCGGAGCGA |
| | (SEQ ID NO: 170) | (N = A, G, C, or T) | (SEQ ID NO: 172) |
| | | (SEQ ID NO: 171) | |
| ACTB | GTGATGGAGGAGGTTTAGTAAGTT | CCAATAAAACCTACTCCTCCCTTAA | ACCACCACCCAACACACAATAACAAACACA |
| | (SEQ ID NO: 55) | (SEQ ID NO: 56) | (SEQ ID NO: 85) |

The PCR reaction system was prepared, containing: 10 μL of the mixed DNA composition (4 ng DNA), 2.5 μL of premixed solution containing the primers, and the probes set forth above; and 12.5 μL of PCR reagent mix (Luna® Universal Probe qPCR Master Mix (NEB)).

The PCR reaction was carried out as follows: 5 mins at 95° C., followed by 50 cycles of 15 seconds at 95° C. and 40 seconds at 56° C. (during which fluorescence was detected). Different fluorescence was detected at the corresponding fluorescent channel, using ABI 7500 Real-Time PCR System.

Results

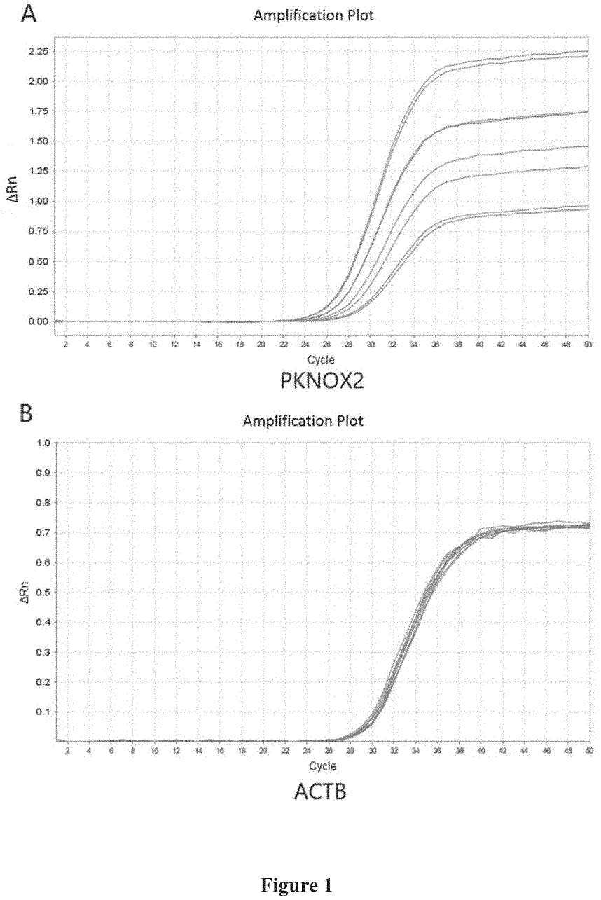

Ct (cycle threshold) values were calculated for each PCR reaction, and Ct values for PCR reactions for each marker with different mixed DNA compositions were analyzed. It was found that, for each marker tested, the pair of the methylation-specific primers used in the PCR reaction provided for Ct values that proportionally decreased as the percentage of the converted methylated DNA increased in the mixed DNA composition. For all tested markers, the percentages of methylated templates have high correlation (correlation coefficient R>0.9 for all tested markers) and linearity with the expected Ct values, which indicated that the primers used for pre-amplifying the target markers were methylation-specific. The correlation can be seen from the horizontal shift of curve as shown in FIG. 1A (obtained with methylation-specific primers for PKNOX2), as compared with the overlapping curves shown in FIG. 1B (obtained with methylation-non-specific primers for control marker ACTB). Results of other methylation-specific primers tested for markers other than PKNOX2 were similar to Figure TA, and were not shown here.

### Example 2: Comparison of Methylation Abundances of Target Markers in Different Tissues

To demonstrate the feasibility and specificity of selected target markers on tumor samples, we tested 28 markers in colorectal cancer tissues (CRC-tissue), advanced adenoma tissues (AA-tissue), paracancerous tissues (para-tissue) from colorectal cancer patients and white blood cells (WBC) from colonscopy negative people as the control. The experimental methods are detailed below.

Methylation abundances of target markers were detected in DNA samples from different cells and tissues, to explore the potential of these target markers in diagnosis or screening for colorectal neoplasm. The target markers tested in this example included, NDRG4, BCAT1, IKZF1, Septin9, SDC2, VAV3, IRF4, TMEFF2, SALL1, BCAN, POU4F2, PKNOX2, ASCL4, KCNA6, SOX1, HS3ST2, FGF12, KCTD8, HMX1, MARCH11, CRHBP, NKX2-6, SLC24A2 and 5 intergenic regions, including INTERGENIC REGION 1, INTERGENIC REGION 2, INTERGENIC REGION 3, INTERGENIC REGION 4, and INTERGENIC REGION 5.

The procedure included the following steps:

1. DNA samples were obtained from white blood cells, paracancerous tissues, advanced adenoma tissues, and colorectal cancer tissues, respectively, with 10 biological samples for each type of sample (i.e. a total of 40 samples). White blood cell DNA was extracted with Qiagen QIAamp DNA Mini Kit, tissue DNA was extracted with Qiagen QIAamp DNA FFPE Tissue Kit by following the instruction of supplier.

2. The DNA samples obtained in step 1 above were treated with a bisulfite reagent (MethylCode™ Bisulfite Conversion Kit) to obtain converted DNA.

3. Fluorescent PCR was performed for the converted DNA. Briefly, the converted DNA obtained from step 2 were amplified by PCR reactions in the presence of methylation-specific primer pairs (see Table 2), and detection probes (see Table 2) specific for NDRG4, BCAT1, IKZF1, Septin9, SDC2, VAV3, IRF4, TMEFF2, SALL1, BCAN, POU4F2, PKNOX2, ASCL4, KCNA6, SOX1, HS3ST2, FGF12, KCTD8, HMX1, MARCH11, CRHBP, NKX2-6, SLC24A2 and 5 intergenic regions, including INTERGENIC REGION 1, INTERGENIC REGION 2, INTERGENIC REGION 3, INTERGENIC REGION 4, and INTERGENIC REGION 5. Control marker ACTB was also amplified in the PCR reaction with methylation-non-specific primers (see Table 2), and detection probes (see Table 2). The detection probes for different markers were labeled with different fluorescence. In the PCR reaction system, each of the primers was at a final concentration of 500 nM and each of the detection probes was at a final concentration of 200 nM.

The PCR reaction system was prepared, containing: 10 μL of the converted DNA, 2.5 μL of premixed solution containing the primers, and the probes set forth above; and 12.5 μL of PCR reagent mix (Luna® Universal Probe qPCR Master Mix (NEB)).

The PCR reaction was carried out as follows: 5 mins at 95° C., followed by 10 cycles of 30 seconds at 95° C. and 60 seconds at 56° C. (during which fluorescence was detected). Different fluorescence was detected at the corresponding fluorescent channel, using ABI 7500 Real-Time PCR System.

4. Ct values were calculated, consolidated and compared for the samples obtained from white blood cells, paracancerous tissues, advanced adenoma tissues, and colorectal cancer tissues. Ct values of un-determined wells were assigned as 50.

Results

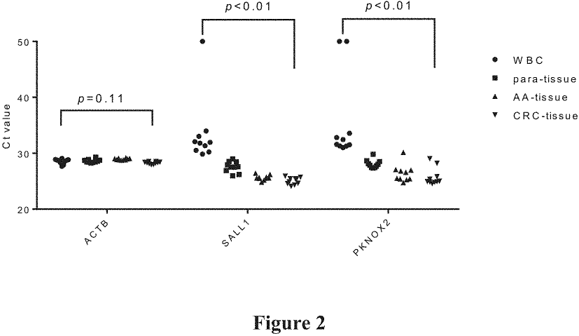

The results showed that the methylation abundances of the target markers of the present disclosure (NDRG4, BCAT1, IKZF1, Septin9, SDC2, VAV3, IRF4, TMEFF2, SALL1, BCAN, POU4F2, PKNOX2, ASCL4, KCNA6, SOX1, HS3ST2, FGF12, KCTD8, HMX1, MARCH11, CRHBP, NKX2-6, SLC24A2 and 5 intergenic regions, including INTERGENIC REGION 1, INTERGENIC REGION 2, INTERGENIC REGION 3, INTERGENIC REGION 4, and INTERGENIC REGION 5) in white blood cells from colonscopy negative people were significantly (p<0.01) lower than in the tissue samples from colon cancer patients (see, FIG. 2), taking SALL1 and PKNOX2 as examples. Significant differences were also observed in each of the other tested target markers (p<0.01), and the results were not shown here. In particular, the methylation abundances of the target markers were lower in paracancerous tissues than in advanced adenoma tissues and colorectal cancer tissues. This showed that each of the target markers as tested have potential application in diagnosis and screening for colorectal neoplasm by using white blood cell samples.

### Example 3: Quantification of Methylated Target Markers by Using Cell-Free DNA

To validate the clinical performance of methylated markers to CRC plasma samples, we tested 13 markers (i.e. NDRG4, Septin9, BCAT1, IKZF1, BCAN, VAV3, POU4F2, SALL1, PKNOX2, SDC2, ASCL4, TMEFF2 and INTERGENIC REGION 1) in 88 clinically diagnosed CRC plasma samples and 107 plasma control samples negative in colonoscopy using the methods disclosed herein (also referred to as Pre-Amplification Method). Among the 88 clinically diagnosed CRC plasma samples, 15 samples were from subjects diagnosed as in CRC Stage I, 26 samples were from subjects diagnosed as in CRC Stage II, 28 samples were from subjects diagnosed as in CRC Stage III, and 19 samples were from subjects diagnosed as in CRC Stage IV.

Pre-Amplification Method

The Pre-Amplification Method included the following steps:

1. Cell-free DNA (cfDNA) samples were obtained from 1-4 ml plasma samples by using QIAamp Circulating Nucleic Acid Kit (Qiagen).

2. 20 ng cfDNA was used as the input for bisulfite conversion with a bisulfite reagent (MethylCode™ Bisulfite Conversion Kit) to obtain converted cfDNA.

3. The converted cfDNA samples were pre-amplified. Briefly, the converted cfDNA obtained from step 2 above were pre-amplified by PCR reactions in the presence of methylation-specific primer pairs (see Table 2), specific for NDRG4, Septin9, BCAT1, IKZF1, BCAN, VAV3, POU4F2, SALL1, PKNOX2, SDC2, ASCL4, TMEFF2 and INTERGENIC REGION 1. In the PCR reaction system, each of the primers was at a final concentration of 200 nM.

The 25 μL PCR mix was composed of 10 μL of the converted cfDNA, 2.5 μL of premixed solution containing the primers set forth above, and 12.5 μL of PCR reagent mix (Luna® Universal Probe qPCR Master Mix (NEB)).

The PCR reaction was carried out as follows: 3 mins at 95° C., followed by 8 cycles of 30 seconds at 95° C. and 60 seconds at 56° C., using ProFlex™ PCR System (Thermo Fisher).

4. The achieved products from step 3 above were diluted by 10-fold and then used for several multiple fluorescent PCR detection, specific for NDRG4, Septin9, BCAT1, IKZF1, BCAN, VAV3, POU4F2, SALL1, PKNOX2, SDC2, ASCL4, TMEFF2 and INTERGENIC REGION 1.

The qPCR mix was composed of 10 μL diluted achieved products from step 3, 2.5 μL primers/probes pool, 12.5 μL of PCR reagent mix (Luna® Universal Probe qPCR Master Mix (NEB)). Non-CpG ACTB region was used as internal control for each reaction well (see Table 2). The detection probes for different markers were labeled with different fluorescence. In the PCR reaction system, each of the primers was at a final concentration of 500 nM, and each of the detection probes was at a final concentration of 200 nM.

The PCR reaction was carried out as follows: 5 mins at 95° C., followed by 50 cycles of 15 seconds at 95° C. and 40 seconds at 56° C. (during which fluorescence was detected). Different fluorescence was detected at the corresponding fluorescent channel, using ABI 7500 Real-Time PCR System.

Results

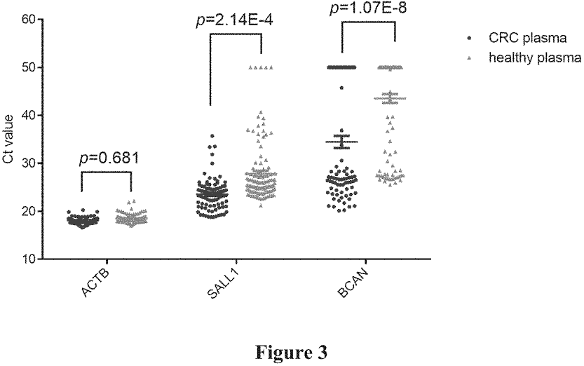

The Ct value was set as 50 for a sample without amplification signal. A reference Ct value was set for each tested marker, respectively. If the Ct value of any one of the tested markers is identical or lower than its corresponding reference Ct value, then the sample would be classified as a positive sample. FIG. 3 shows the Ct value distribution of target markers SALL1 and BCAN in population with CRC and population negative in colonoscopy. As shown in FIG. 3, the methylation levels of target markers SALL1 and BCAN in population with CRC were significantly (p value=2.14E-4 and 1.07E-8 for SALL1 and BCAN, respectively) higher than that in population negative in colonoscopy. The results for the other target markers were similar (p<0.01), and were not shown.

Table 3 below shows the comparison results by using 5 target markers (i.e. Septin9, BCAT1, IKZF1, BCAN and VAV3) in the Pre-Amplification Method. As shown in Table 3, the Pre-Amplification Method showed ultra high sensitivity (86.4%) for CRC and high specificity (90.7%) for population negative in colonoscopy, which greatly outperformed than existing commercialized markers, e.g. Septin9, which has a sensitivity of 48.2% for CRC in clinical trial samples (see T. R. Church et al., Gut.; 63:317-325 (2014)). The other marker combinations (e.g. the combination of Septin9, BCAT1, IKZF1, VAV3, BCAN, and NDRG4; the combination of Septin9, BCAT1, IKZF1, VAV3, BCAN, NDRG4, SDC2, PKNOX2, and TMEFF2, the combination of Septin9, BCAT1, IKZF1, VAV3, BCAN, NDRG4, SDC2, PKNOX2, TMEFF2, and INTERGENIC REGION 1, etc.) within the 13 target markers have been analyzed, and the results showed that the sensitivity for CRC is no less than 85%, and the specificity for population negative in colonoscopy is no less than 90%.

TABLE 3
Comparison of the results between the Pre-
Amplification Method and colonoscopy

| Results of | | Number of | Number of | Total |
|---|---|---|---|---|
| Colonoscopy | Accuracy | Positive Samples | Negative Samples | Number |
| Colorectal | 86.4% | 76 | 12 | 88 |
| Cancer | | | | |
| Negative in | 90.7% | 10 | 97 | 107 |
| colonoscopy | | | | |

The sensitivities of the Pre-Amplification Method and Septin9 Alone Method in classifying CRC were also compared. The Septin9 Alone Method was performed similar to the Pre-Amplification Method, except that the target marker is Septin9 only.

As shown in Table 4, in the Pre-Amplification Method, the sensitivity of CRC Stage I, Stage II, Stage III, and Stage IV was 73.3%, 80.8%, 89.3%, and 100%, respectively. In contrast, in the Septin9 Alone Method, the sensitivity of CRC Stage I, Stage II, Stage III, and Stage IV was 26.7%, 65.4%, 75.0%, and 79%, respectively. Therefore, the Pre-Amplification Method showed a significant increase in sensitivity comparing with the Septin9 Alone Method.

TABLE 4
Comparison of the results between the Pre-
Amplification Method and Septin9 Alone Method

| | Results of the Pre- | | | | |
|---|---|---|---|---|---|
| | Amplification Method | | | | Results of |
| Clinical | | Number of | Number of | | Septin9 Alone |
| Classi- | | Positive | Negative | Total | Method |
| fication | Accuracy | Samples | Samples | Number | Accuracy |
| Stage I | 73.3% | 11 | 4 | 15 | 26.7% |
| Stage II | 80.8% | 21 | 5 | 26 | 65.4% |
| Stage III | 89.3% | 25 | 3 | 28 | 75.0% |
| Stage IV | 100.0% | 19 | 0 | 19 | 79.0% |

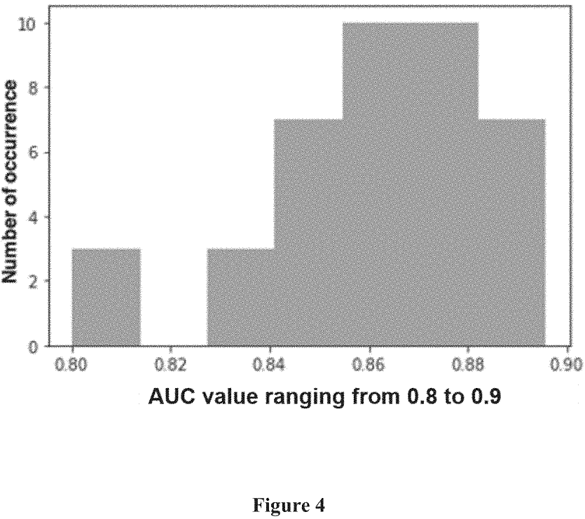

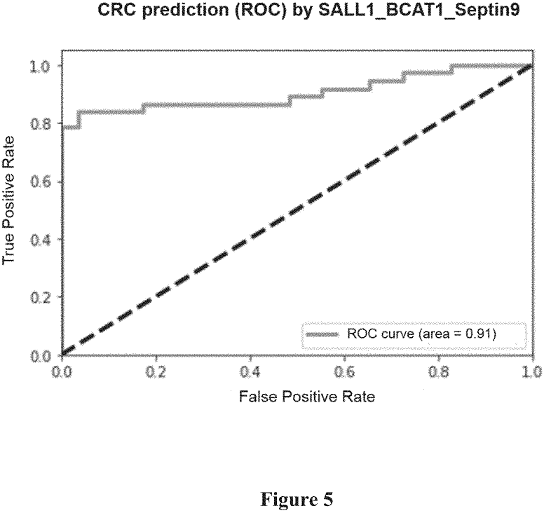

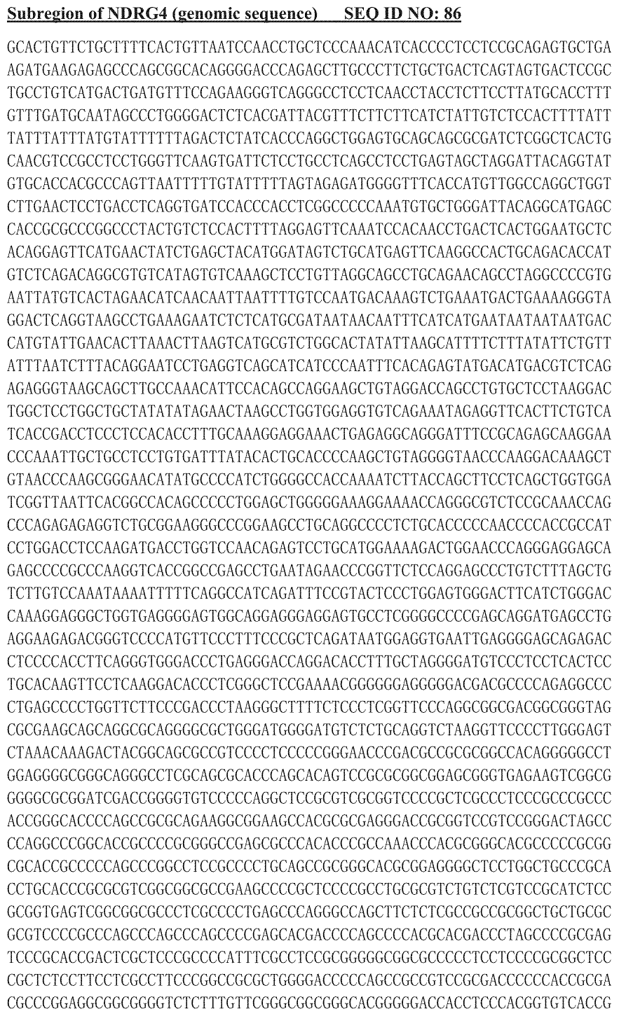

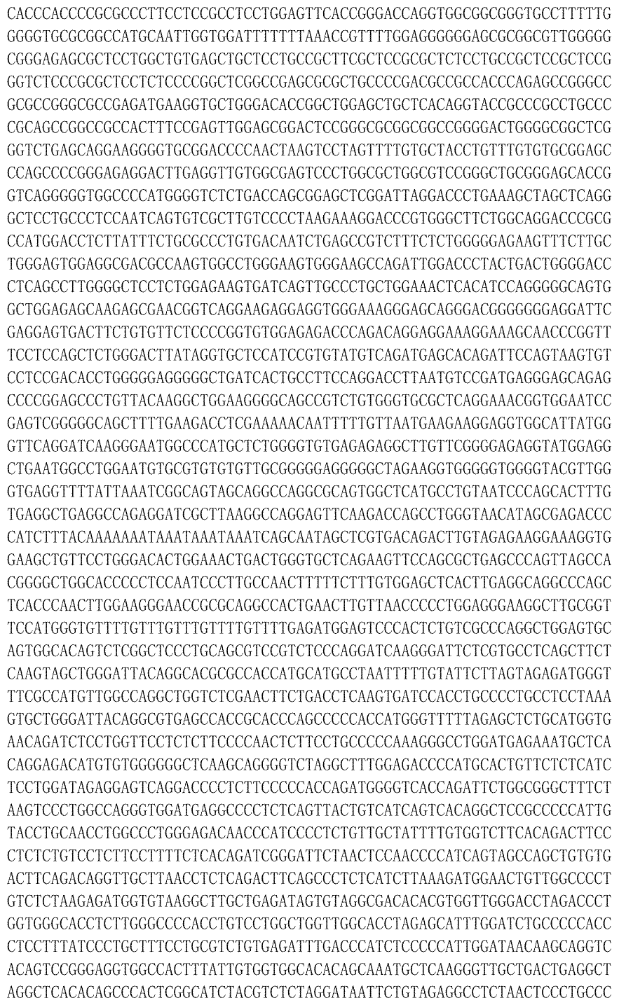

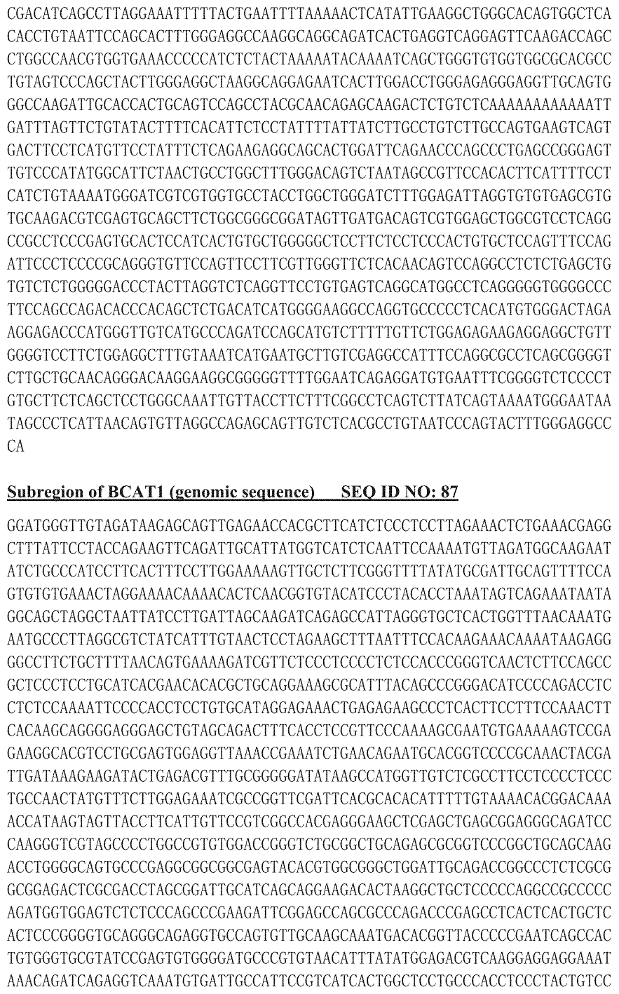

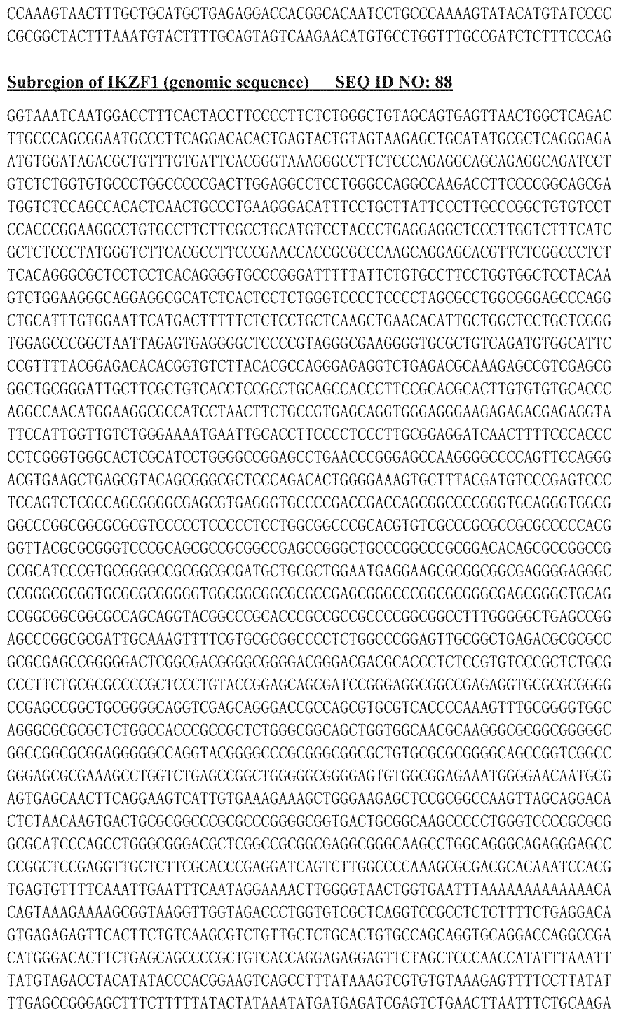

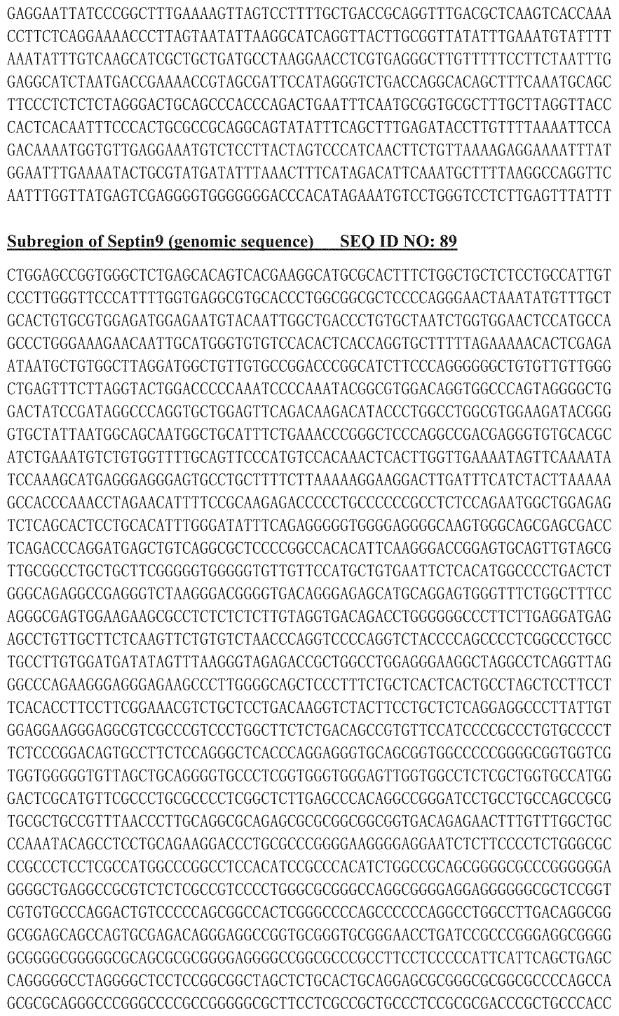

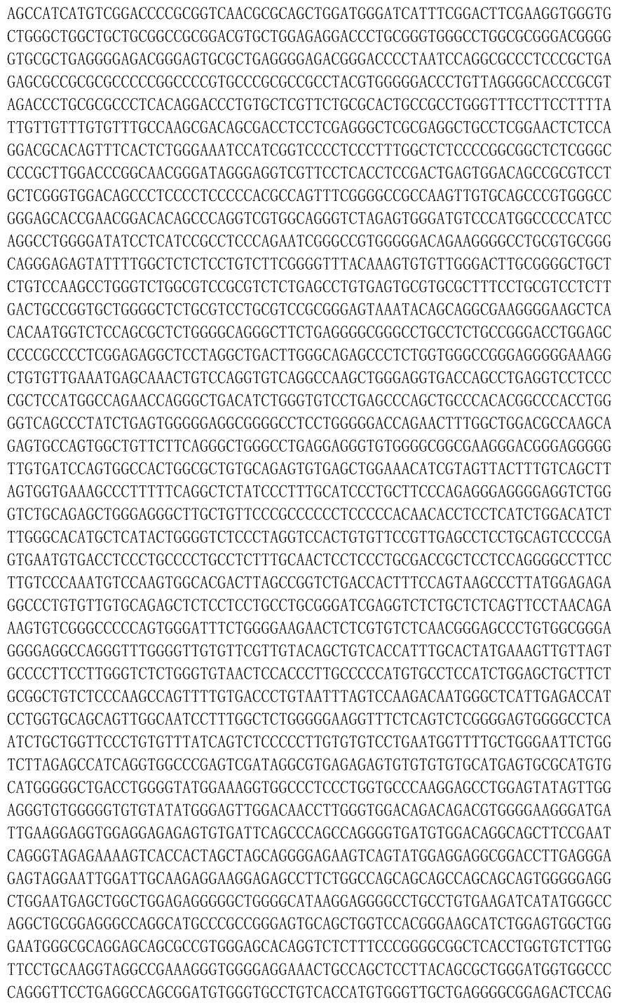

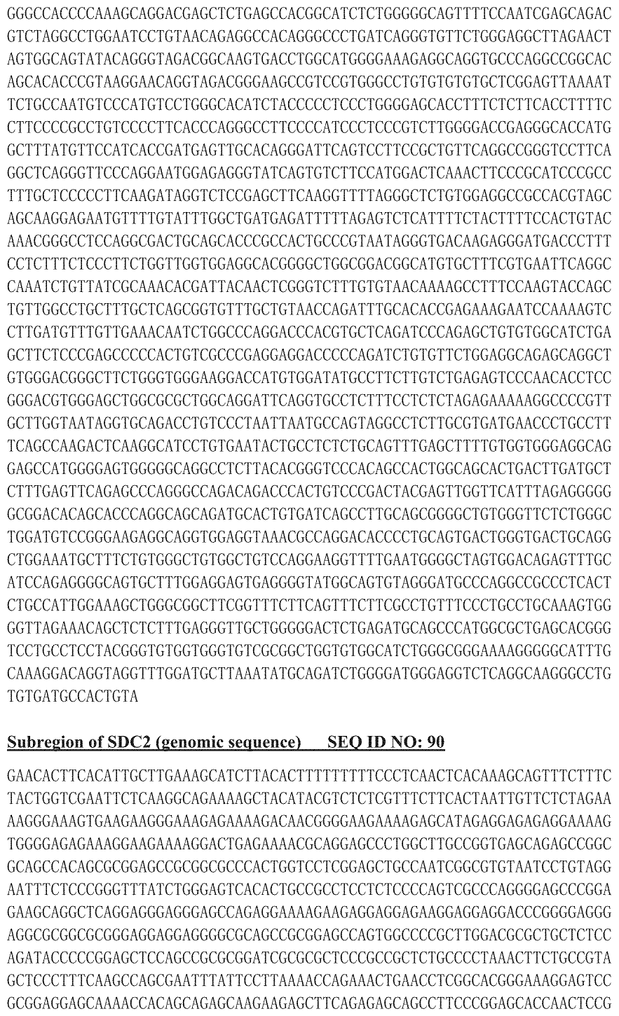

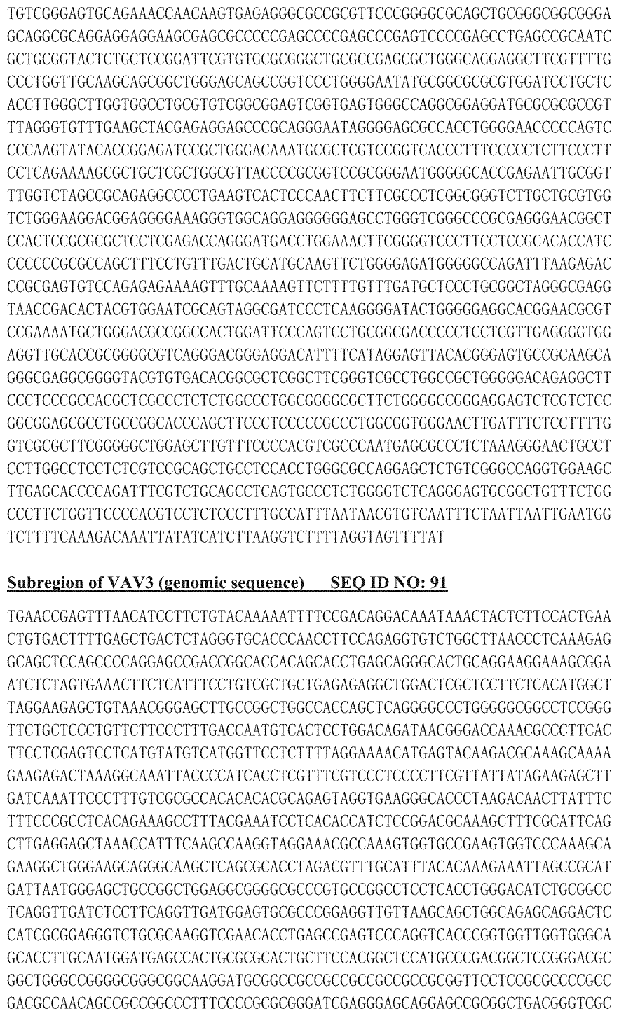

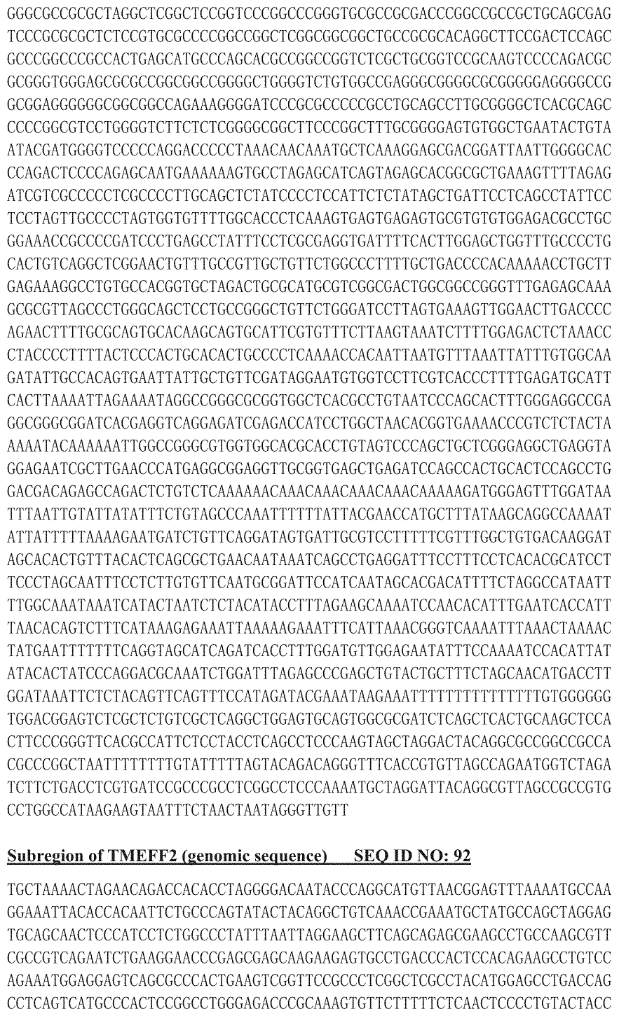

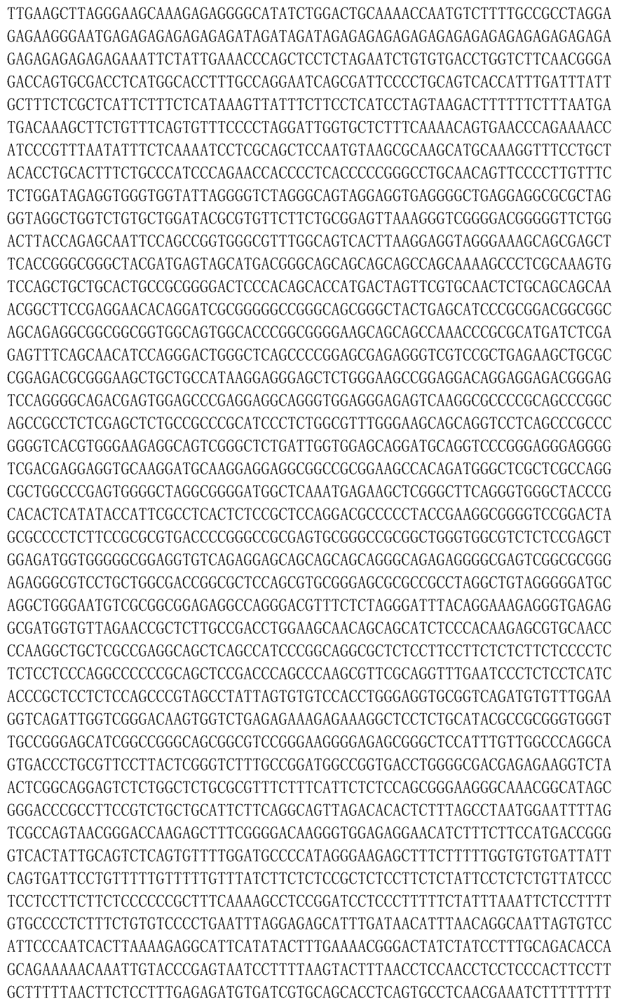

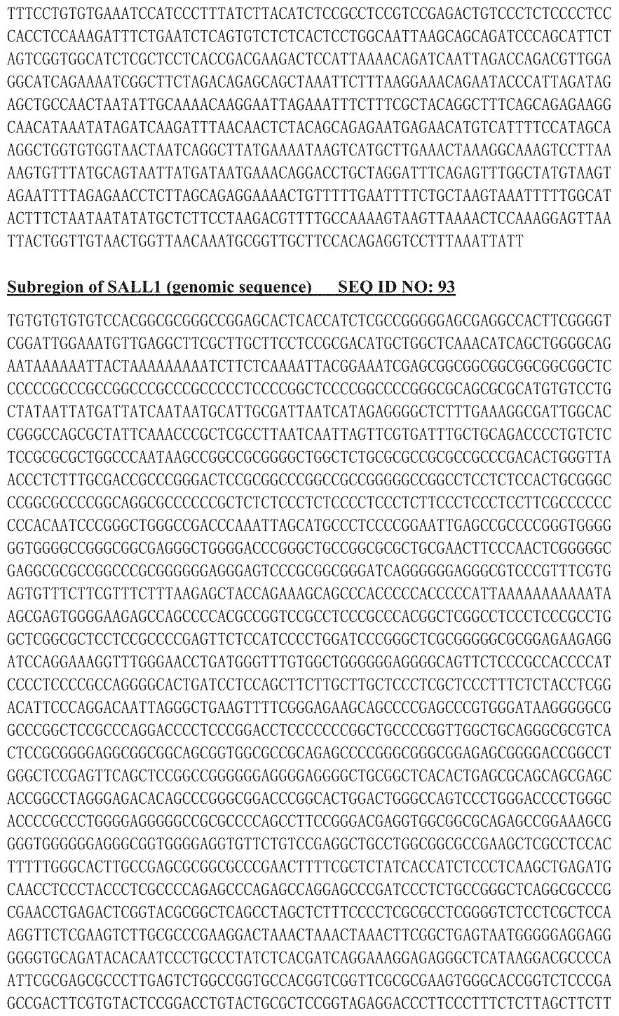

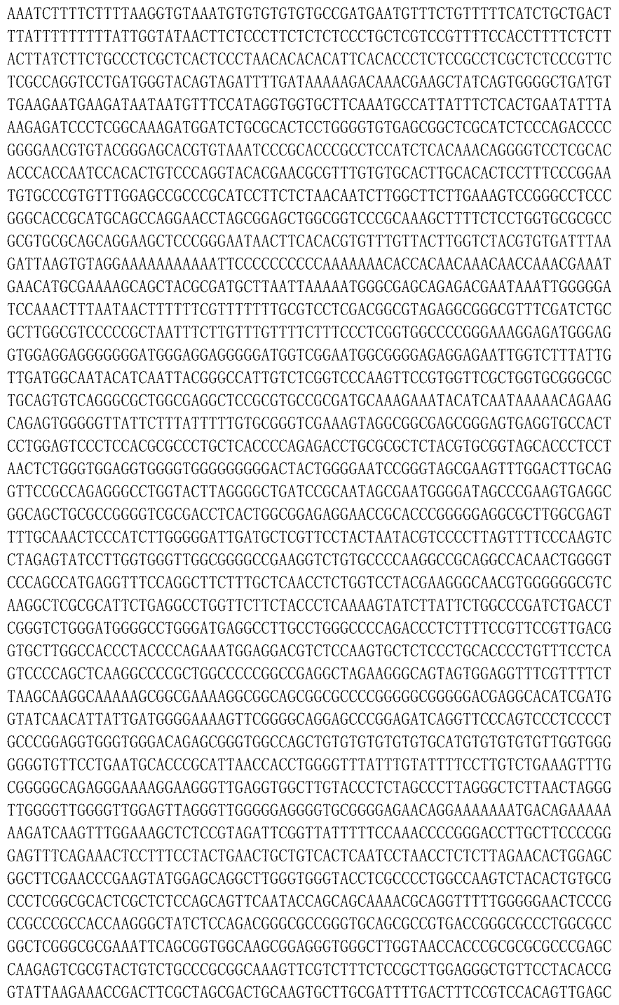

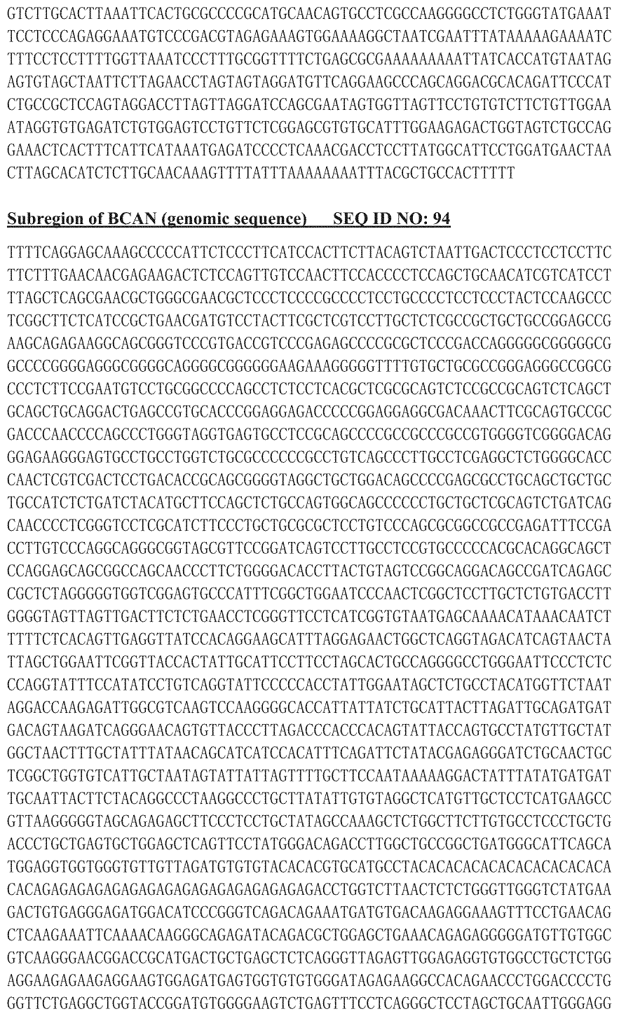

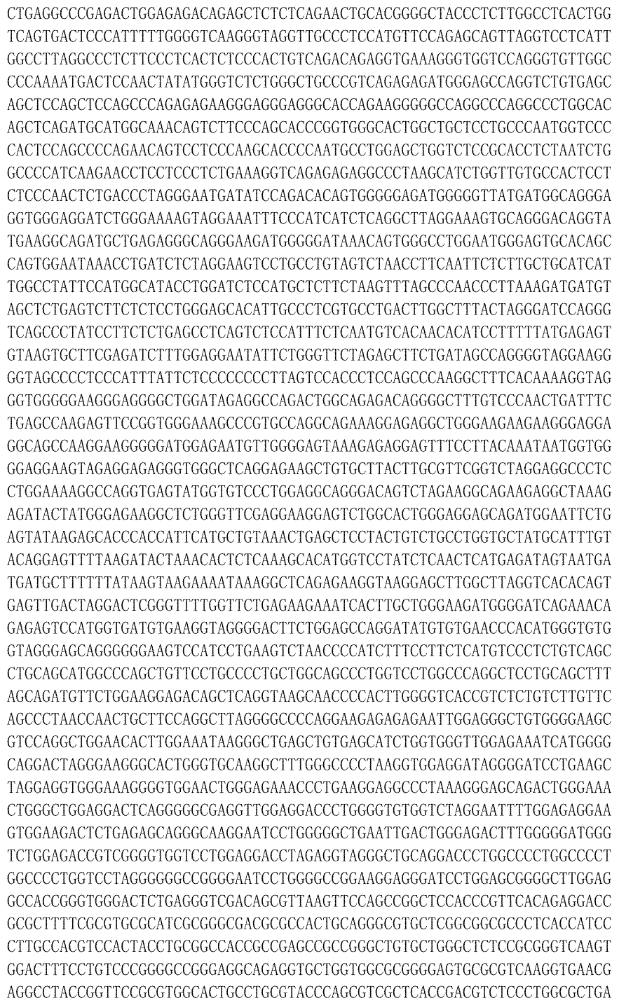

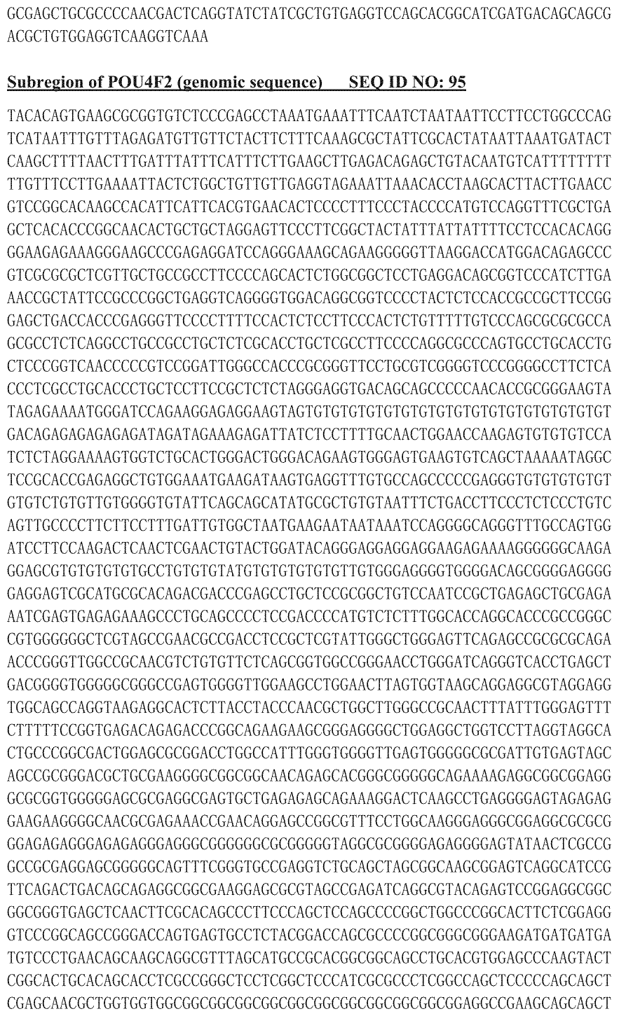

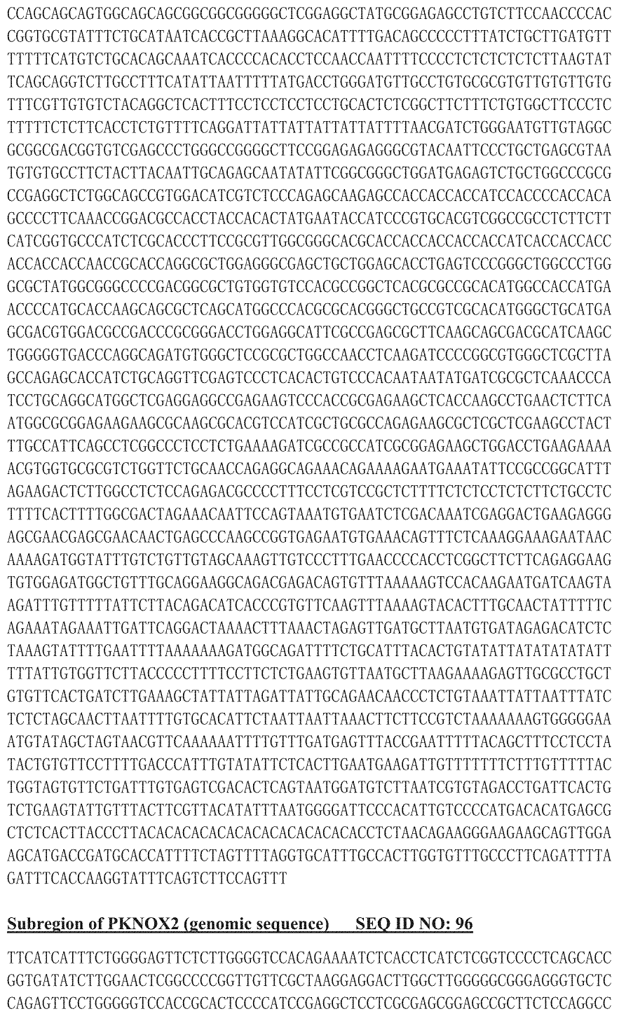

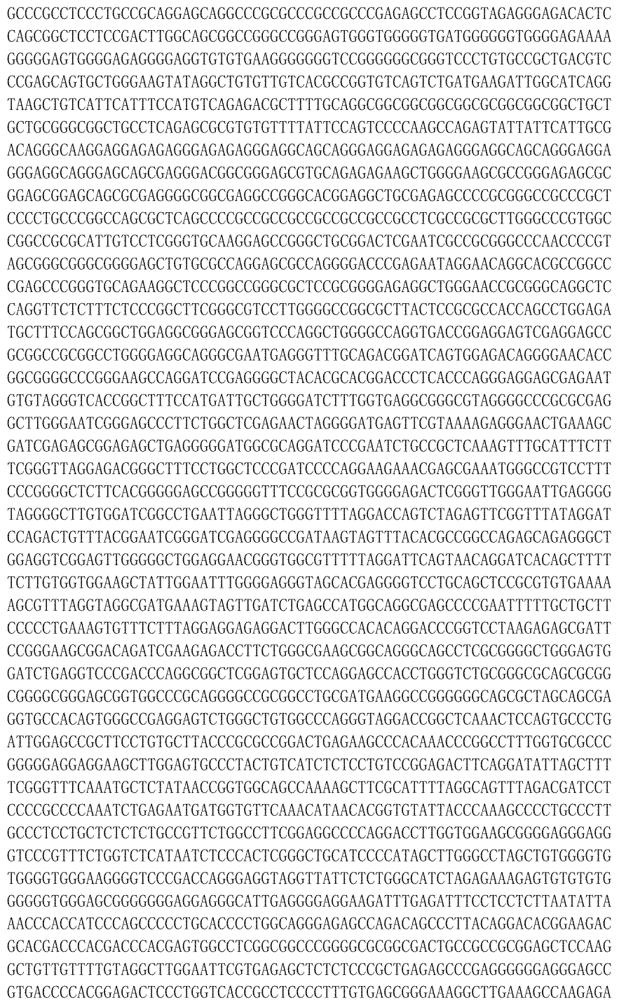

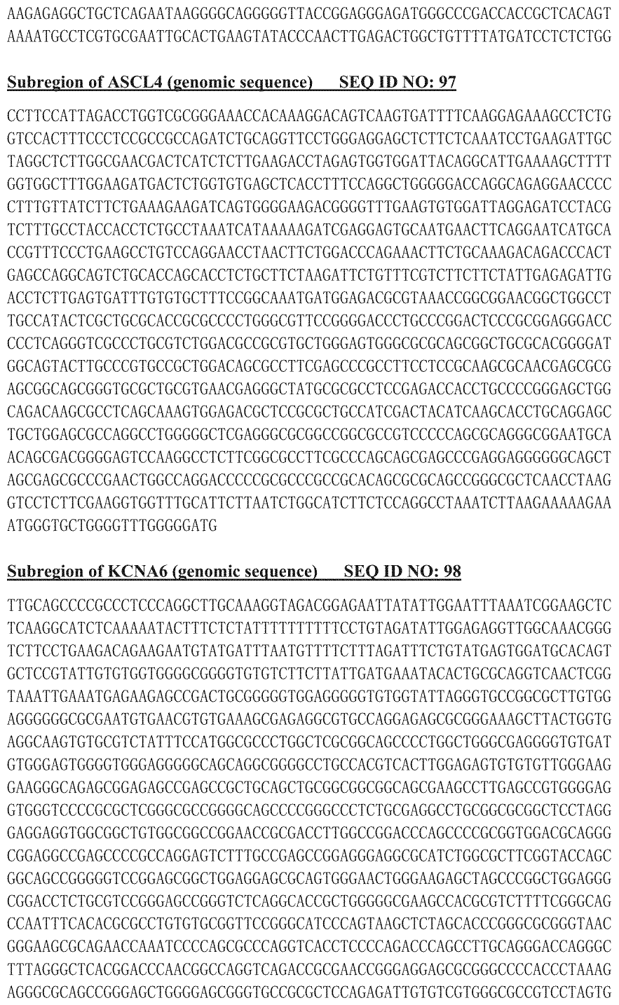

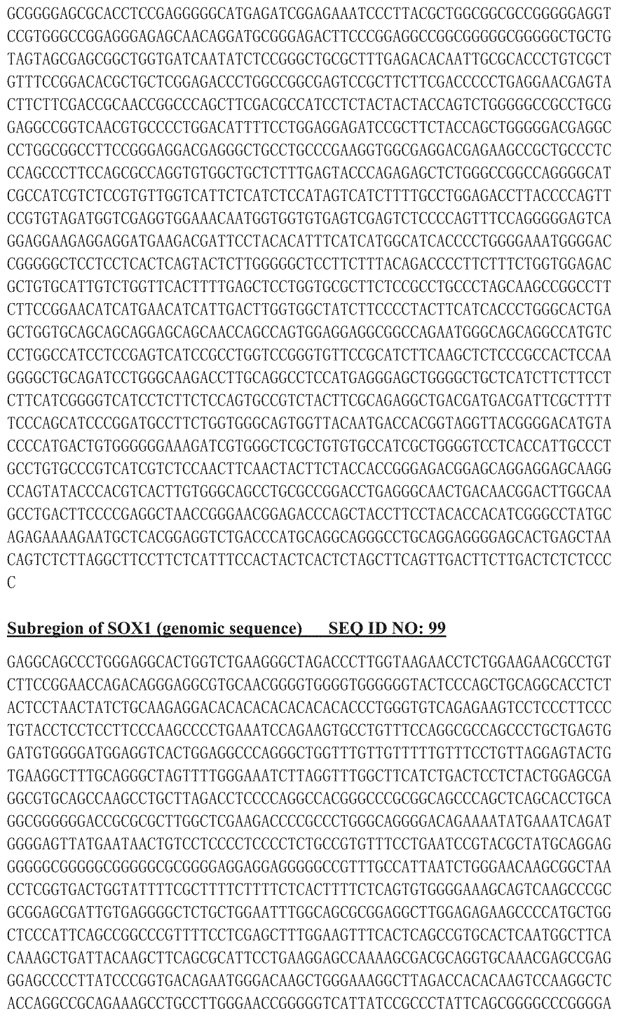

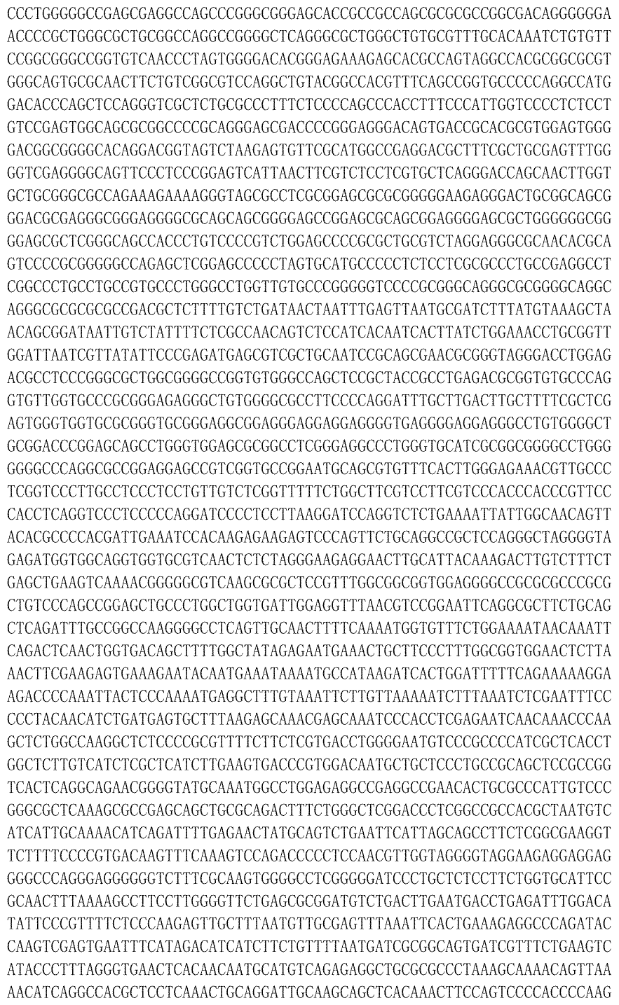

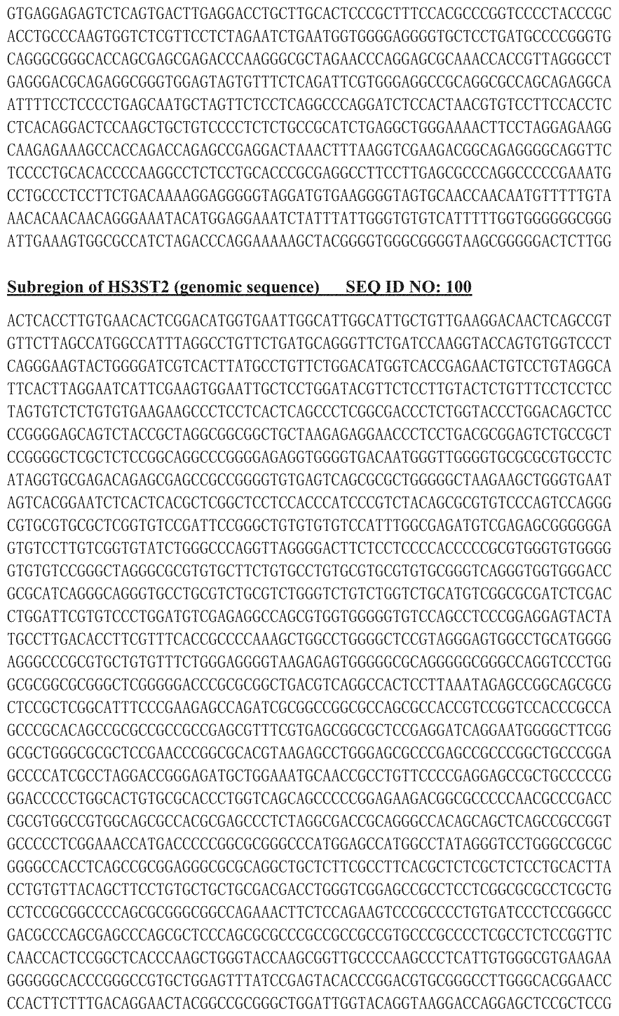

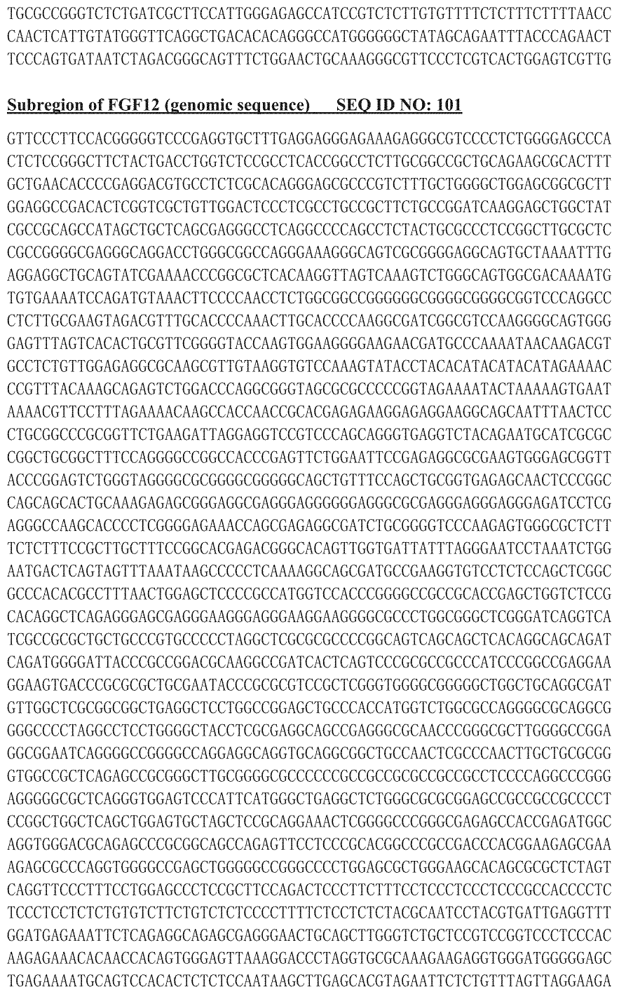

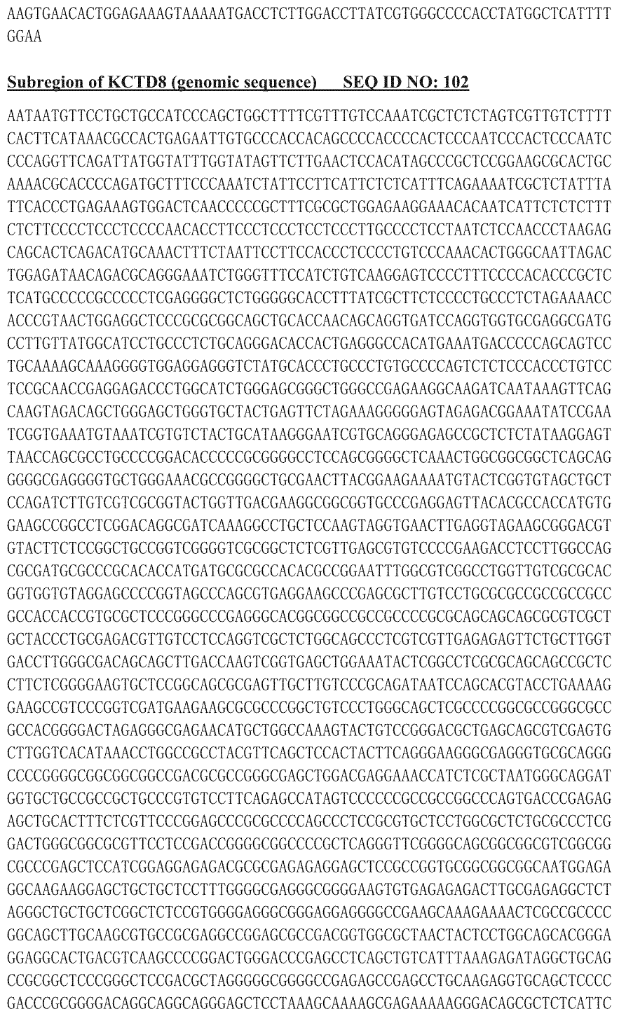

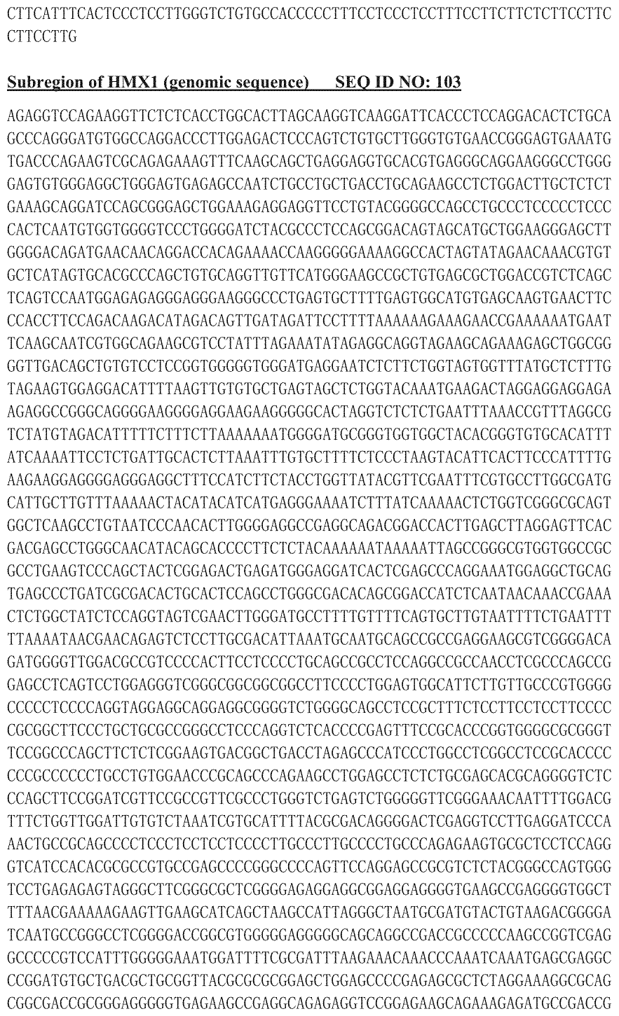

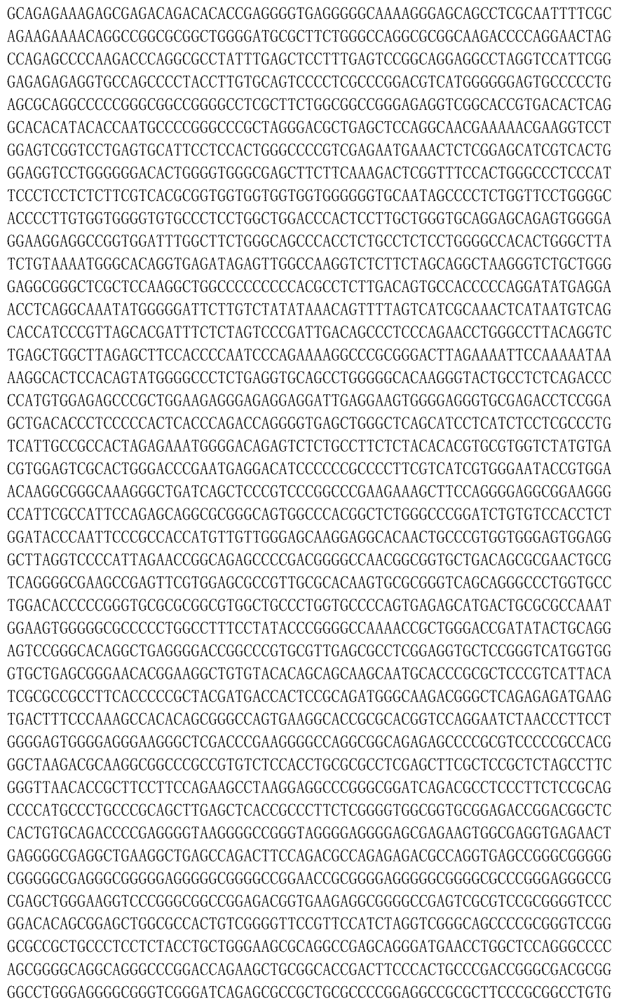

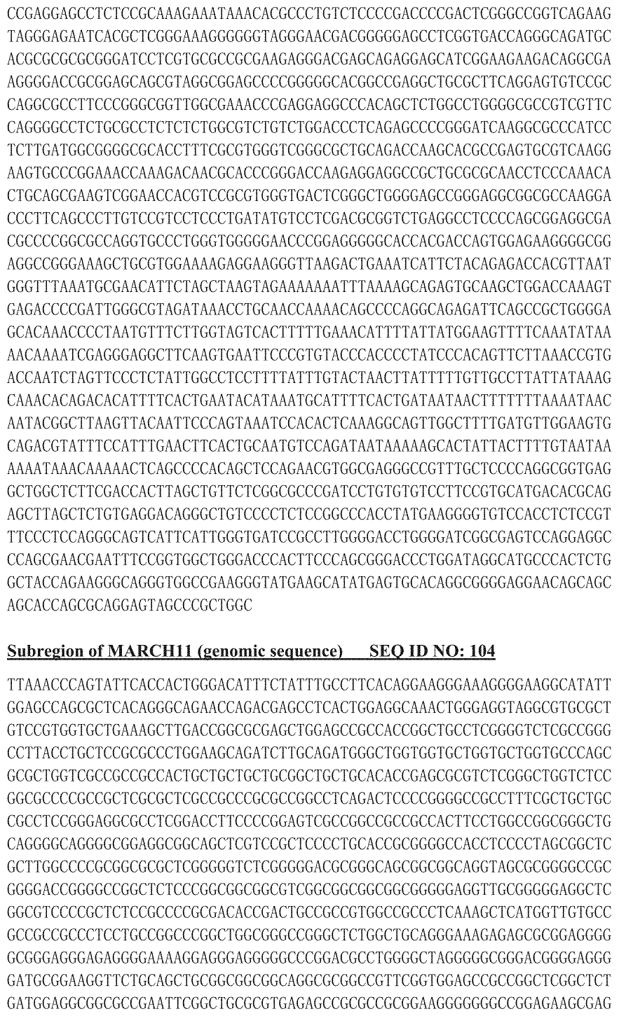

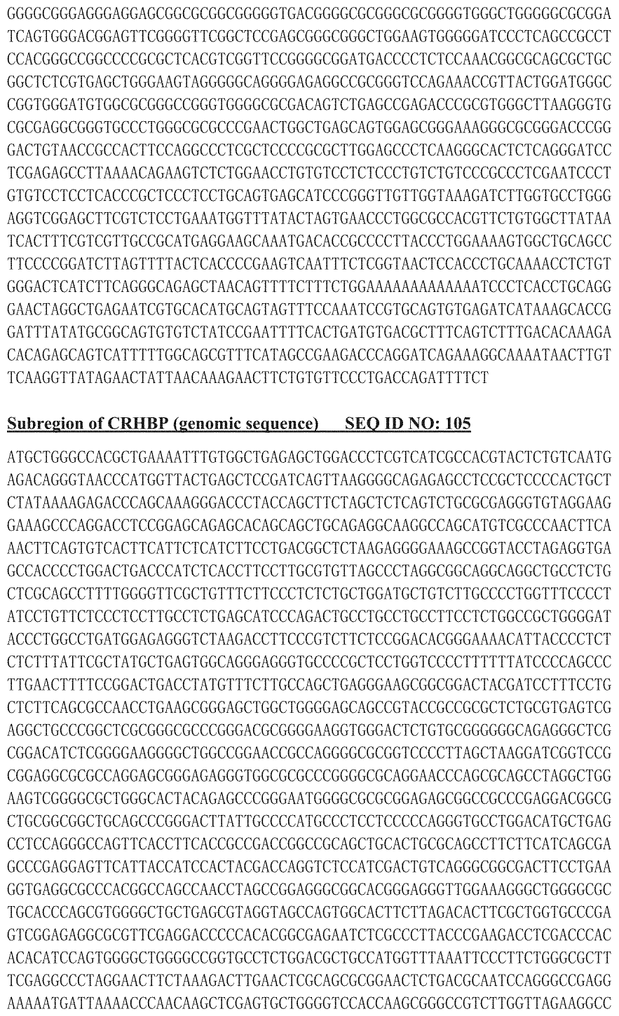

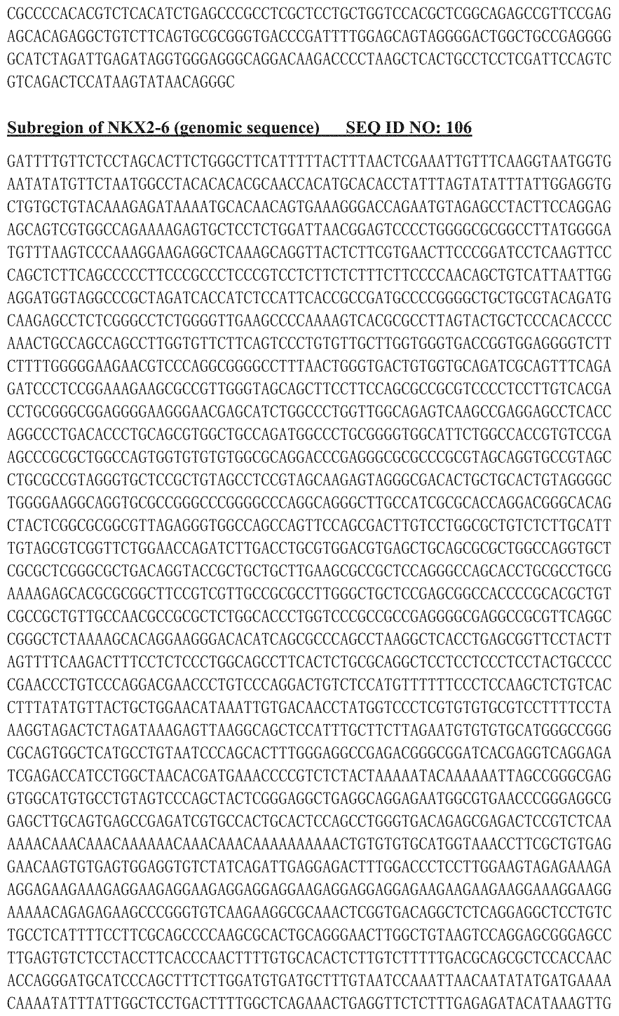

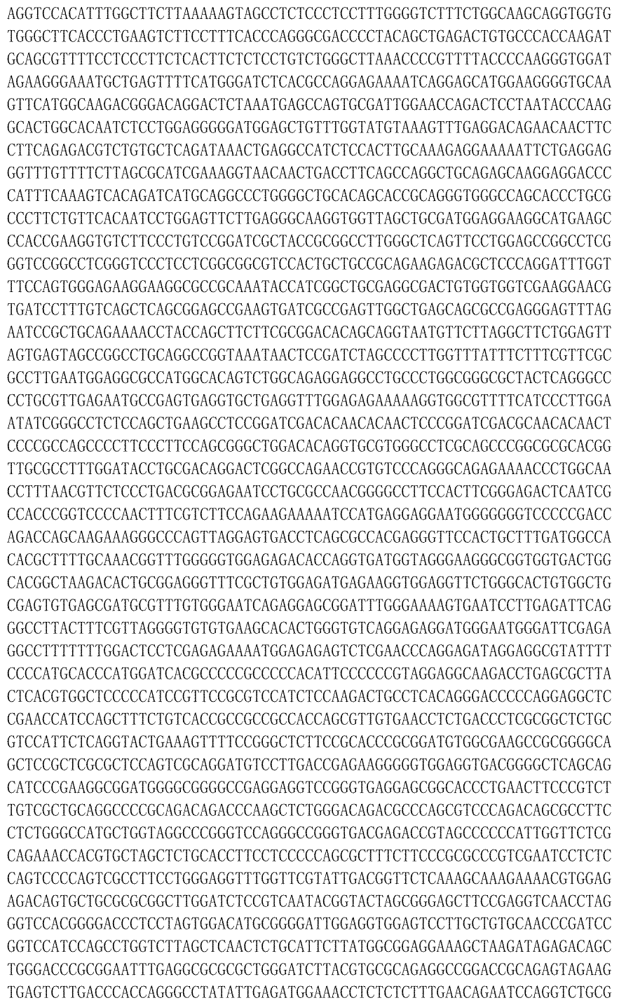

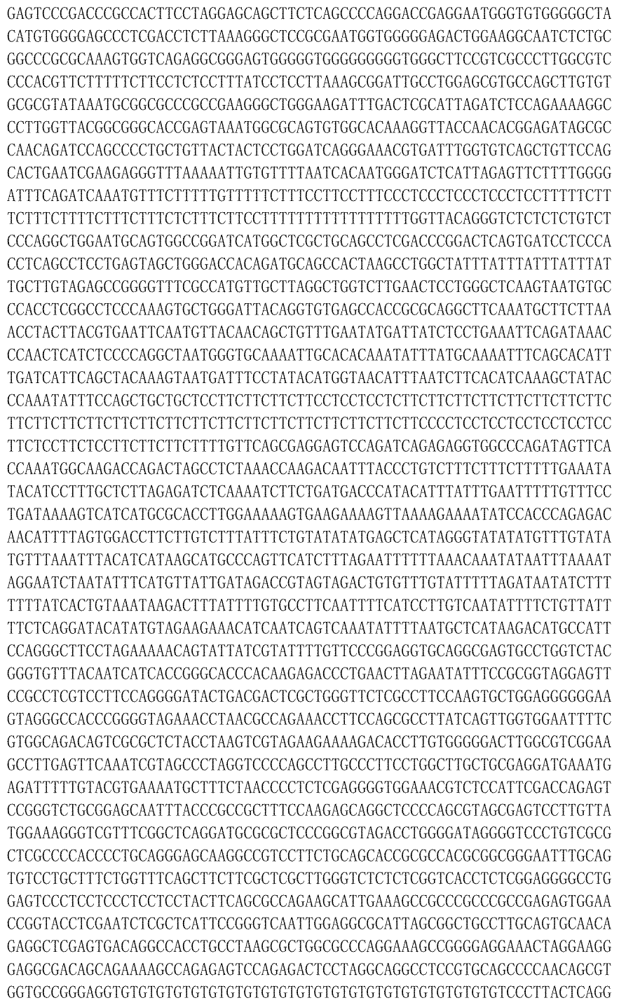

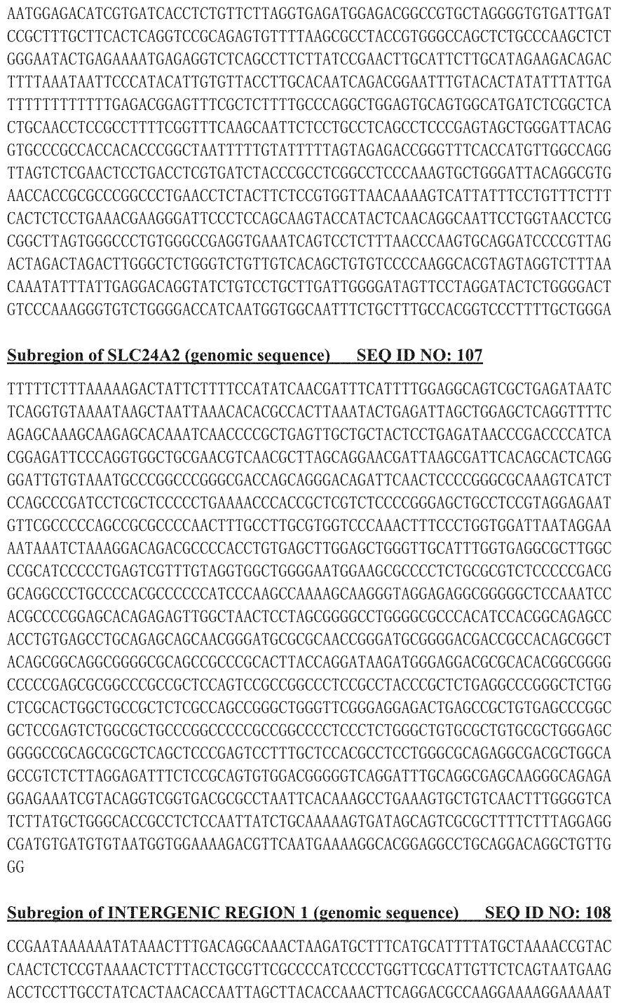

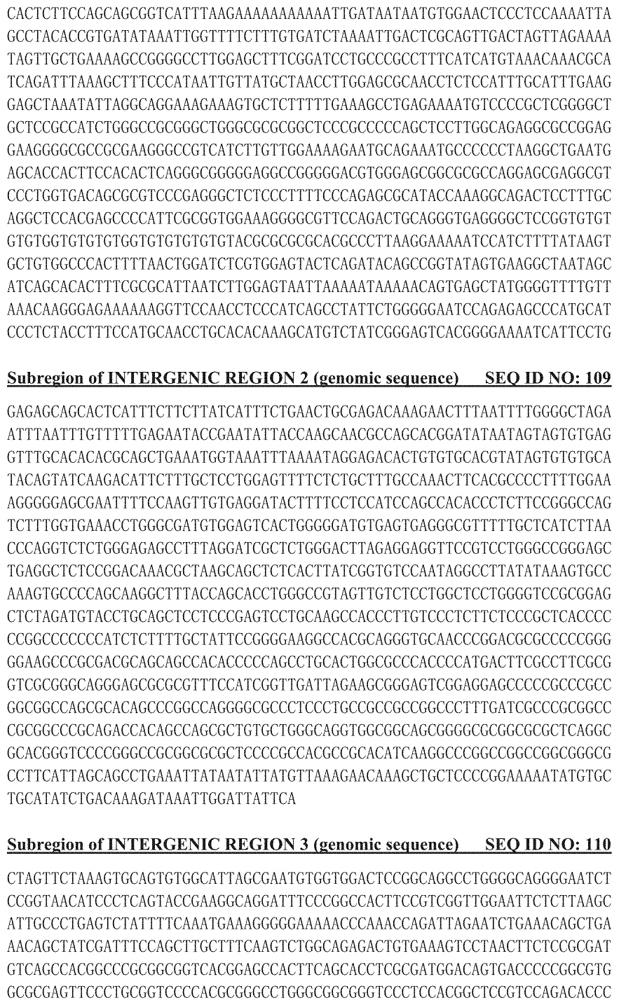

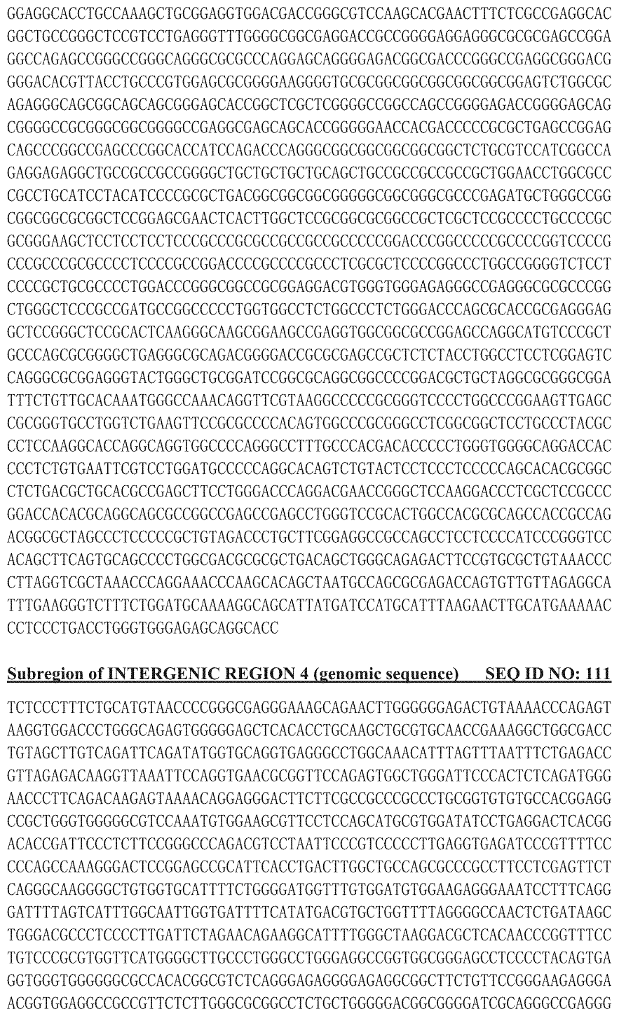

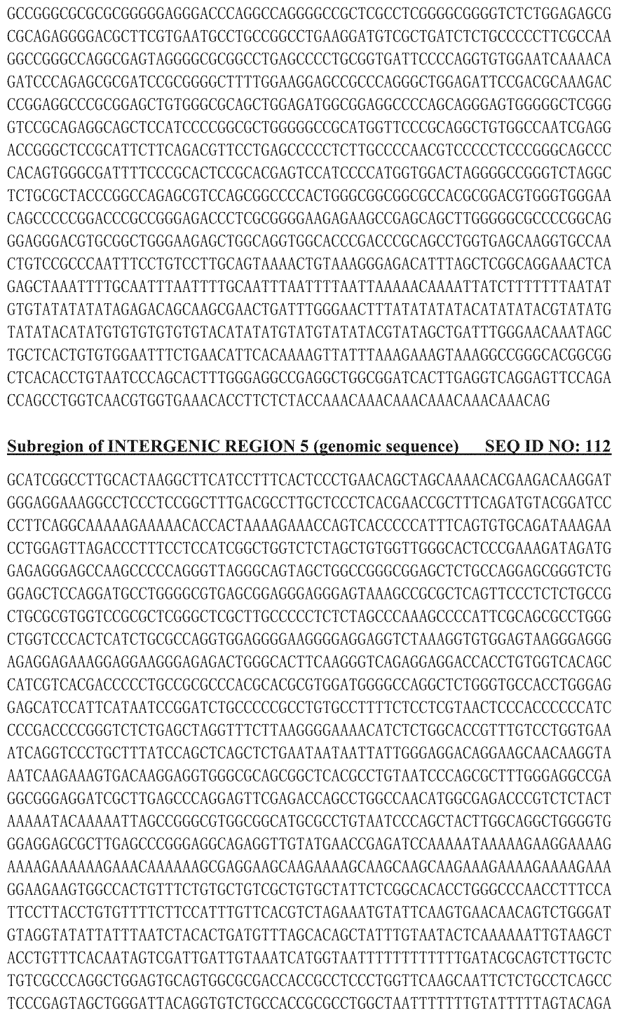

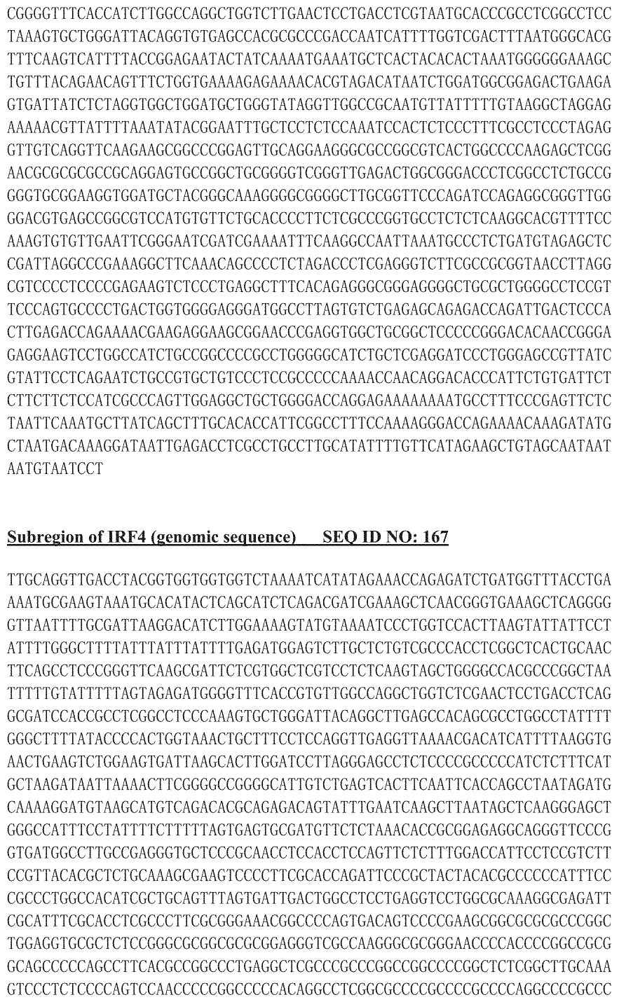

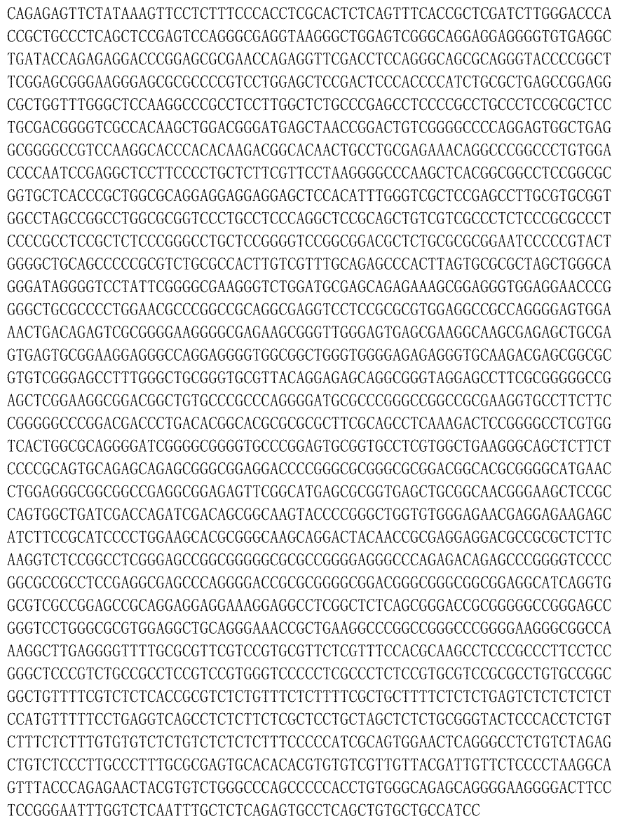

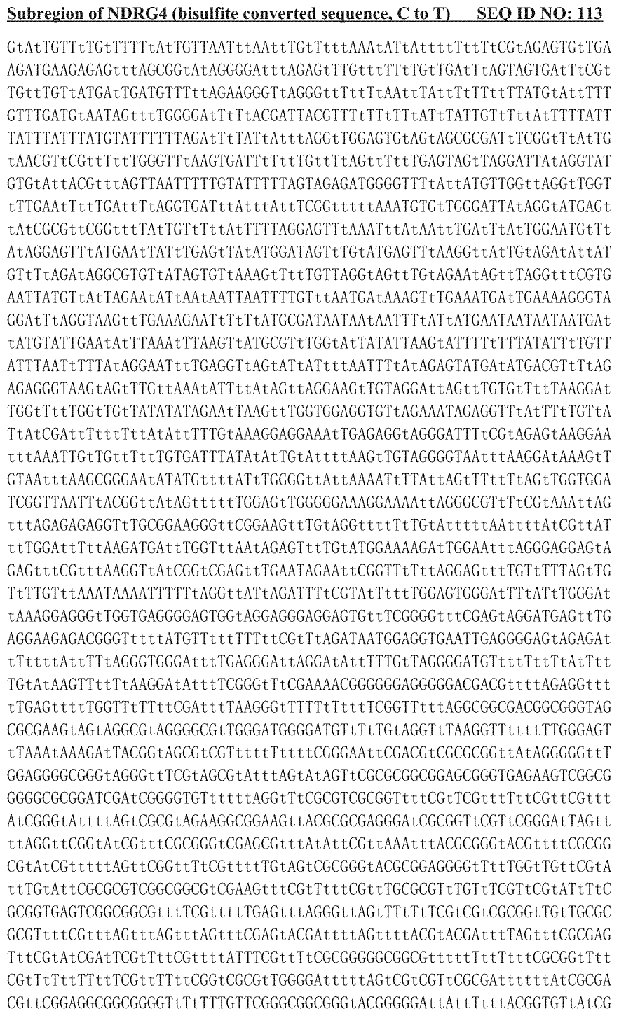

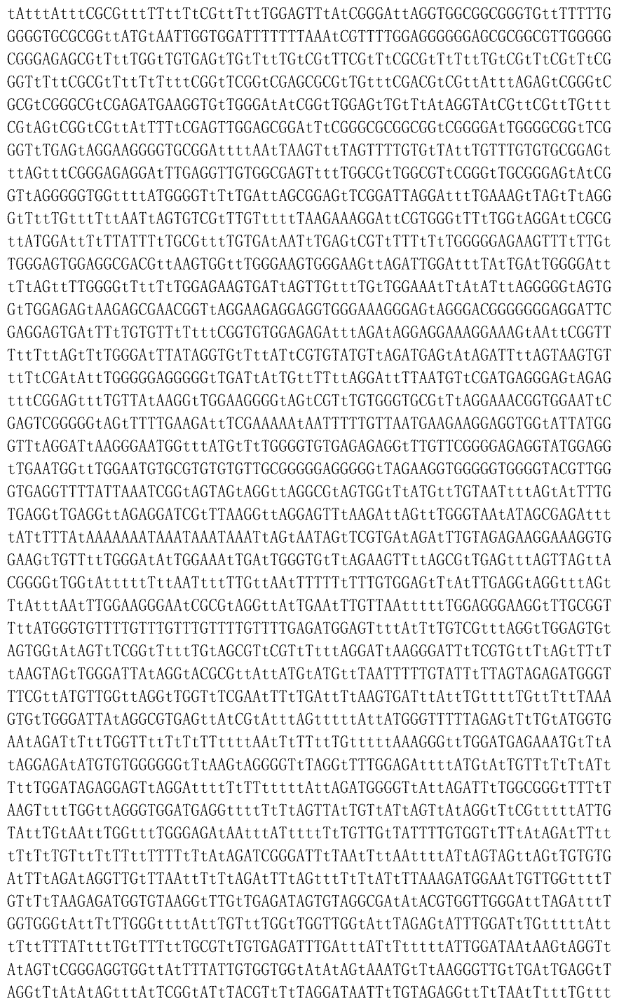

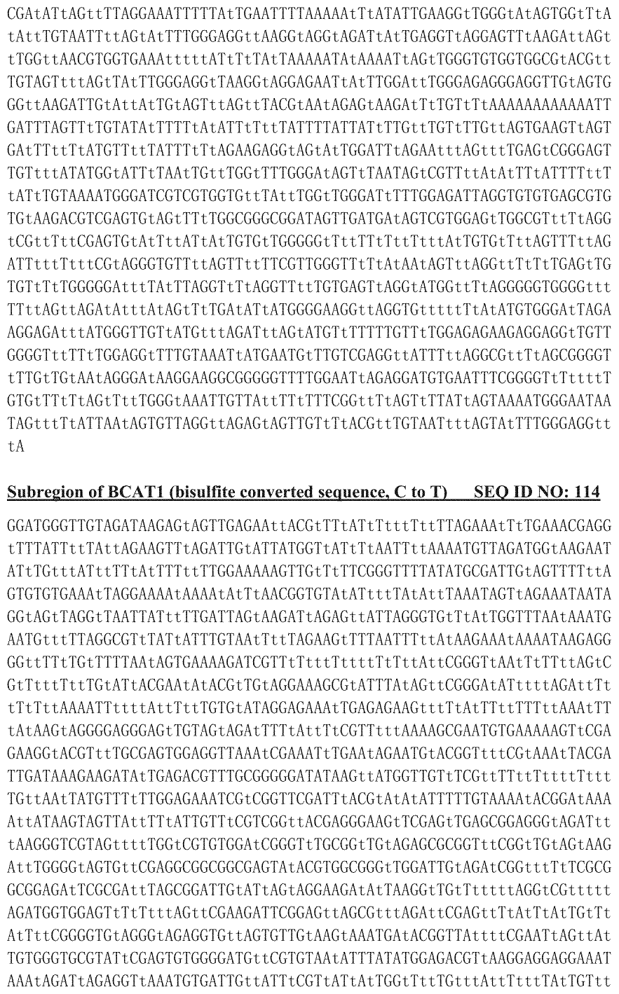

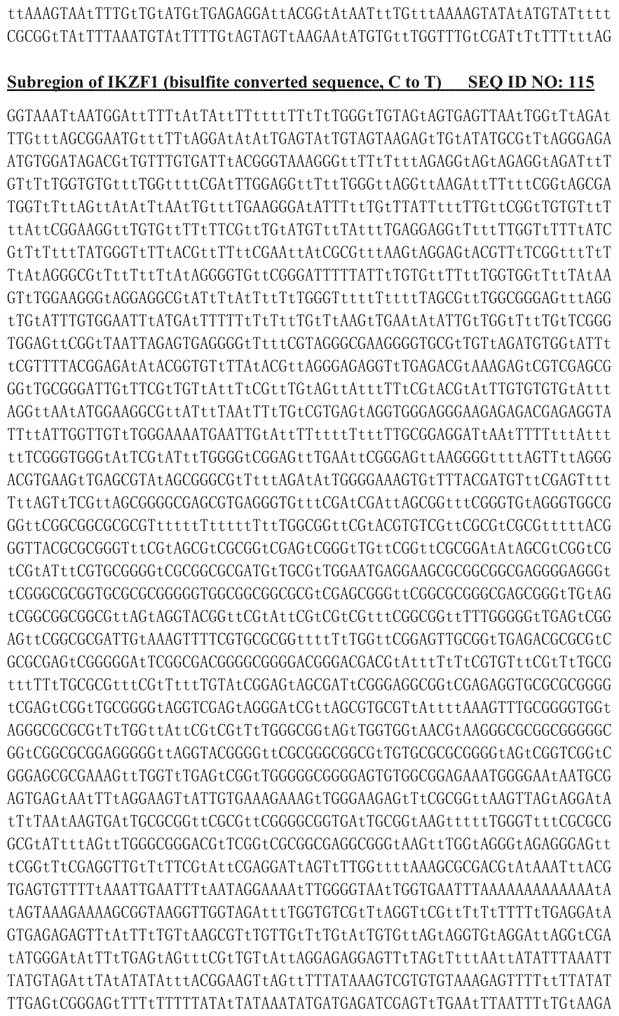

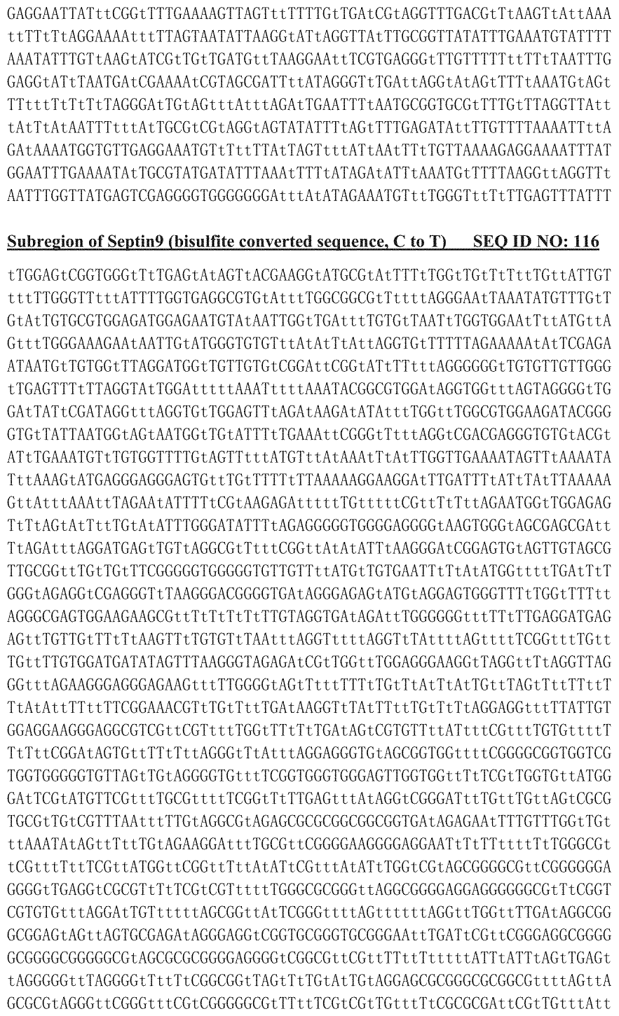

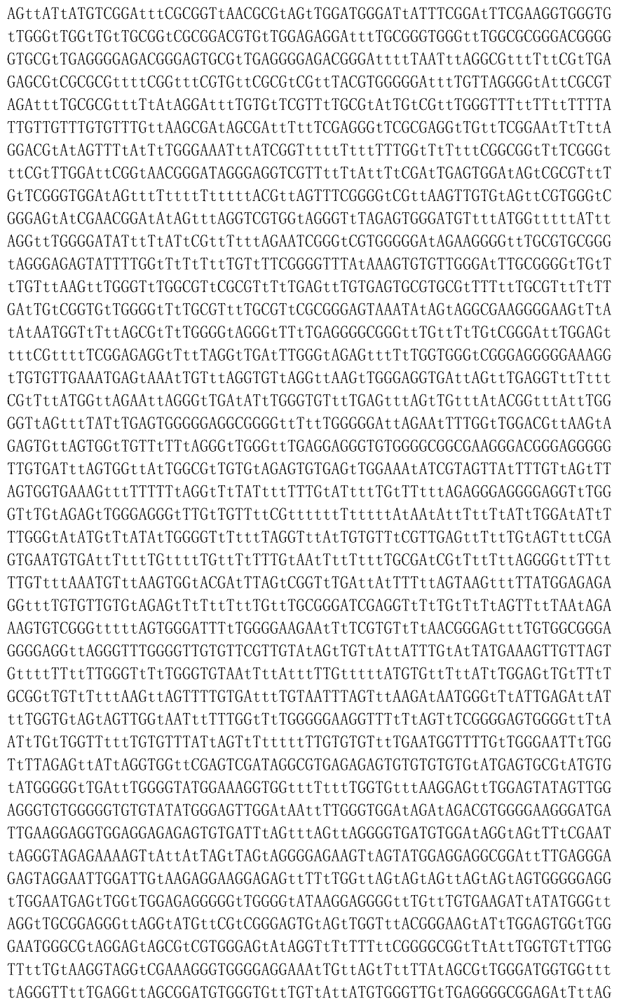

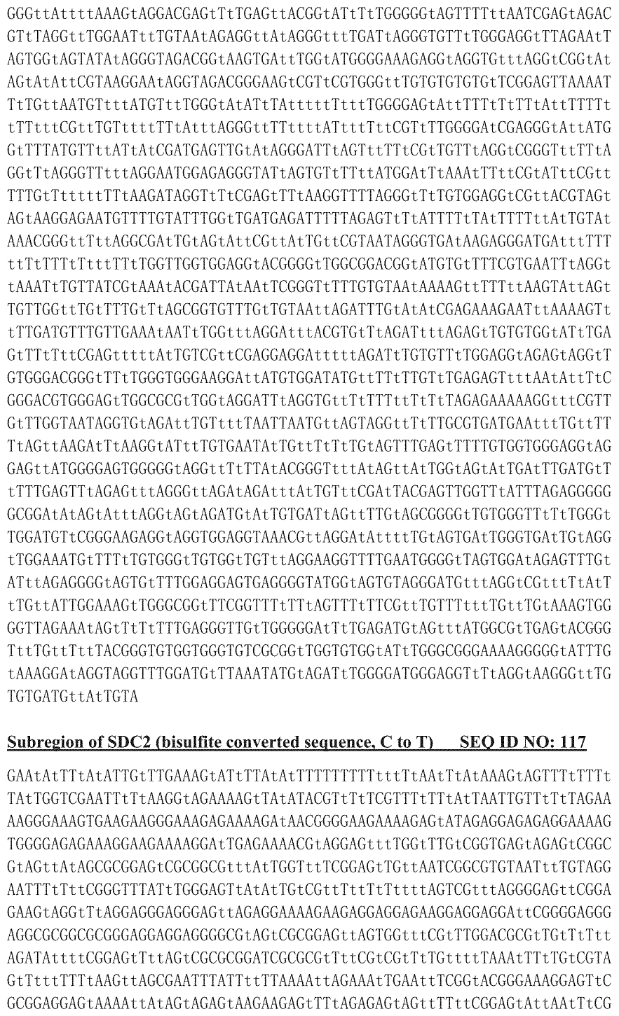

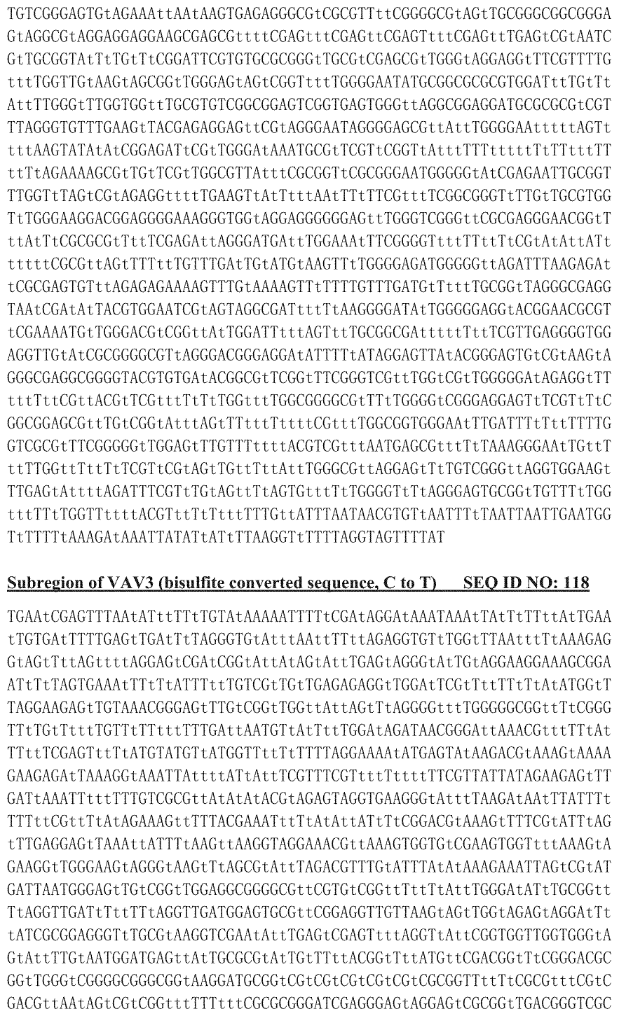

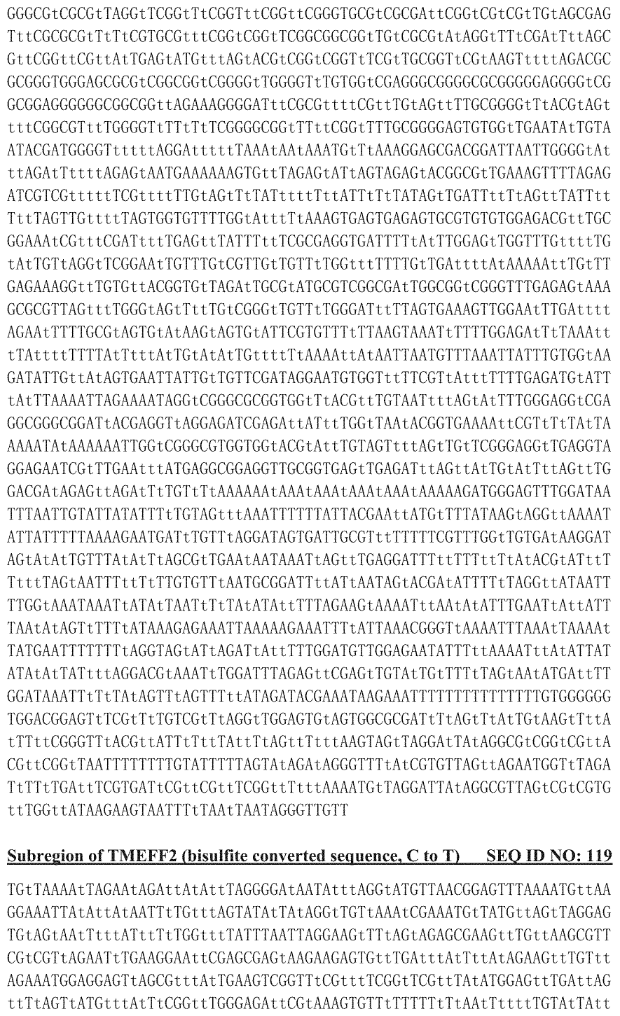

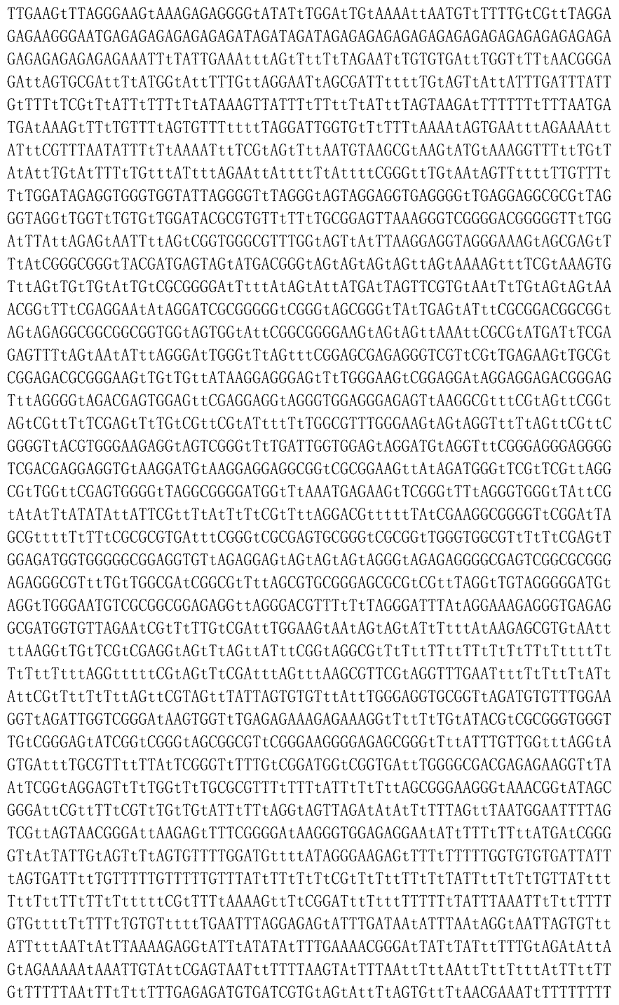

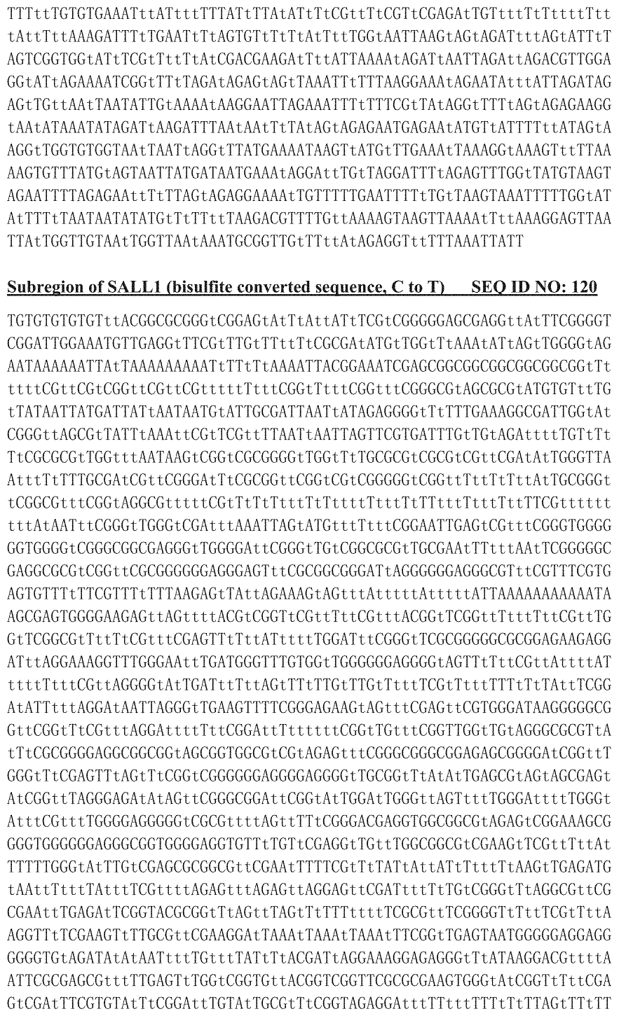

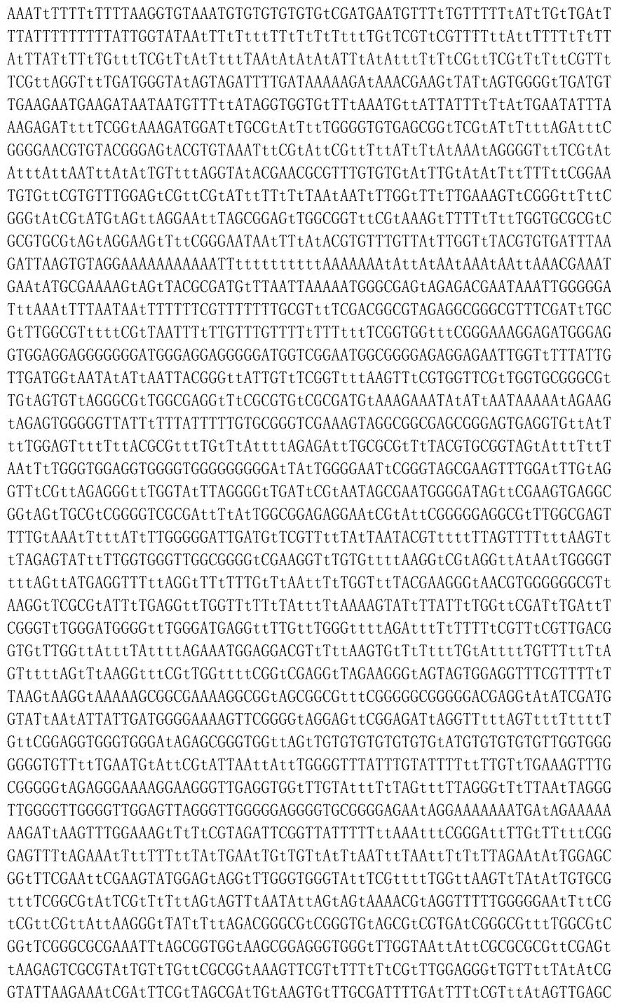

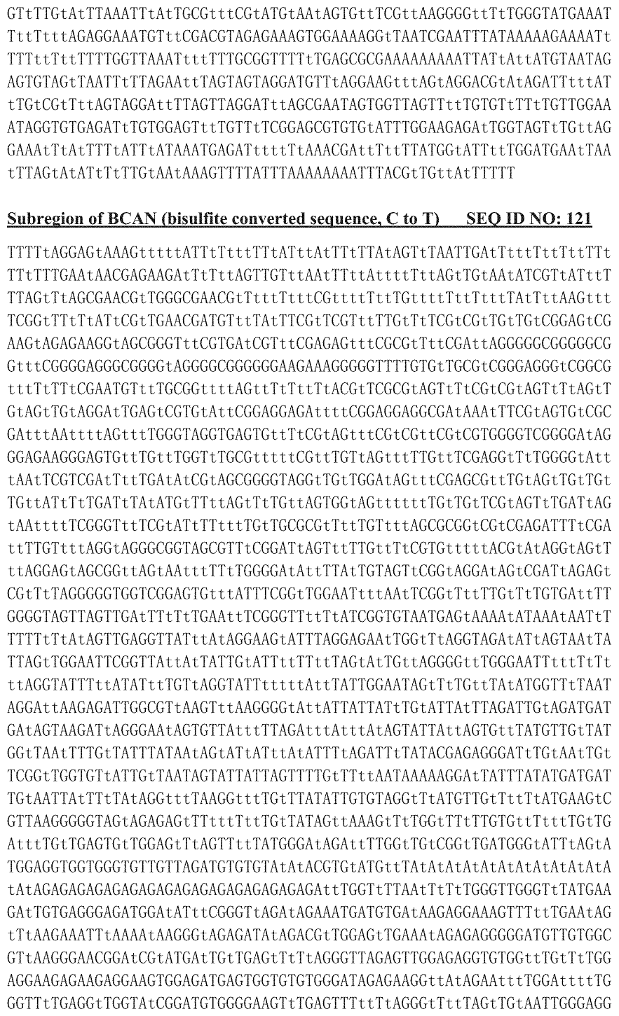

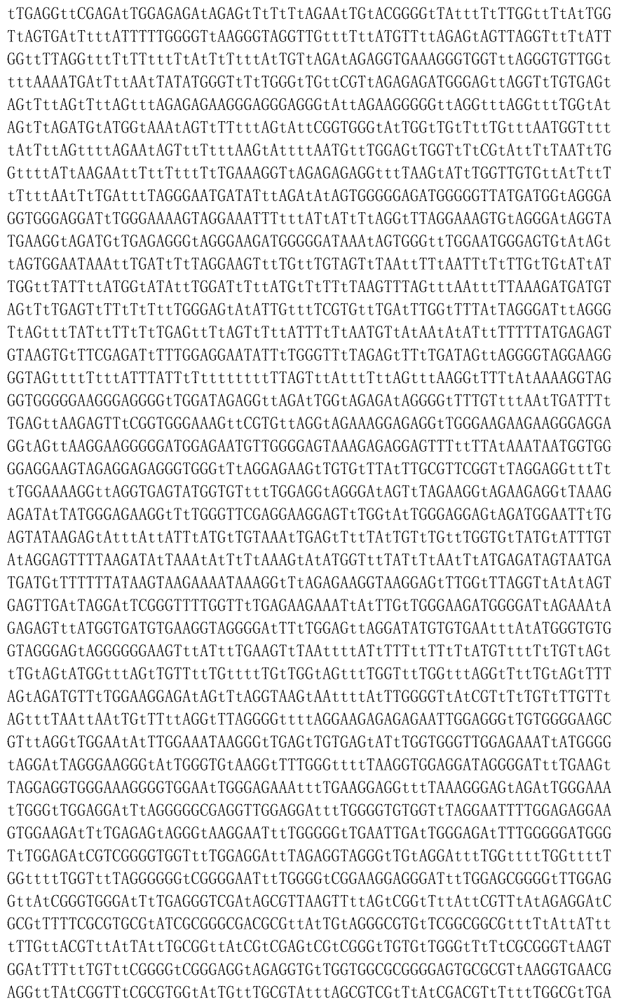

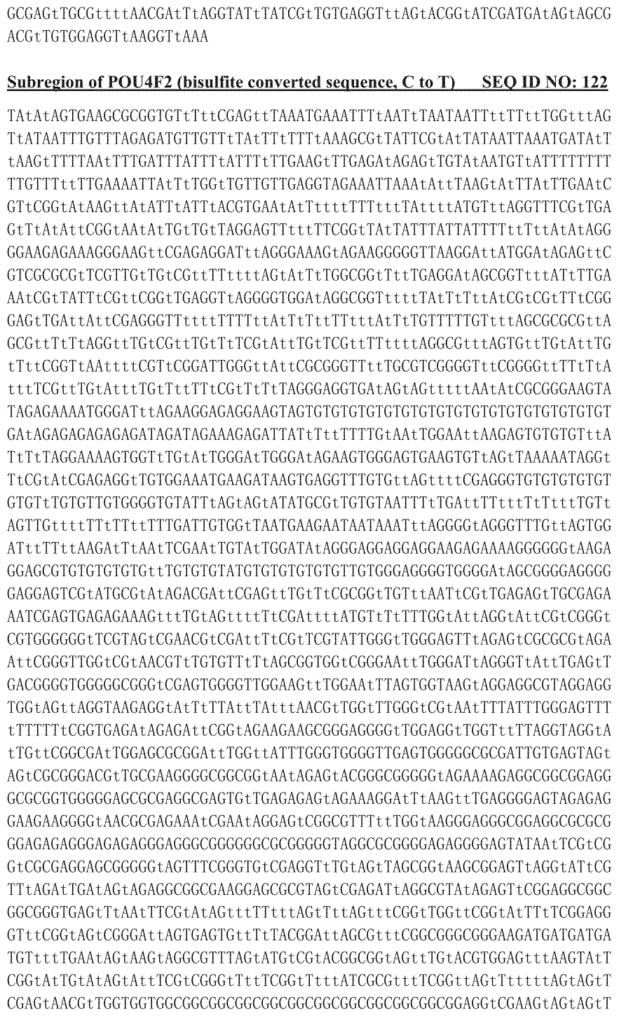

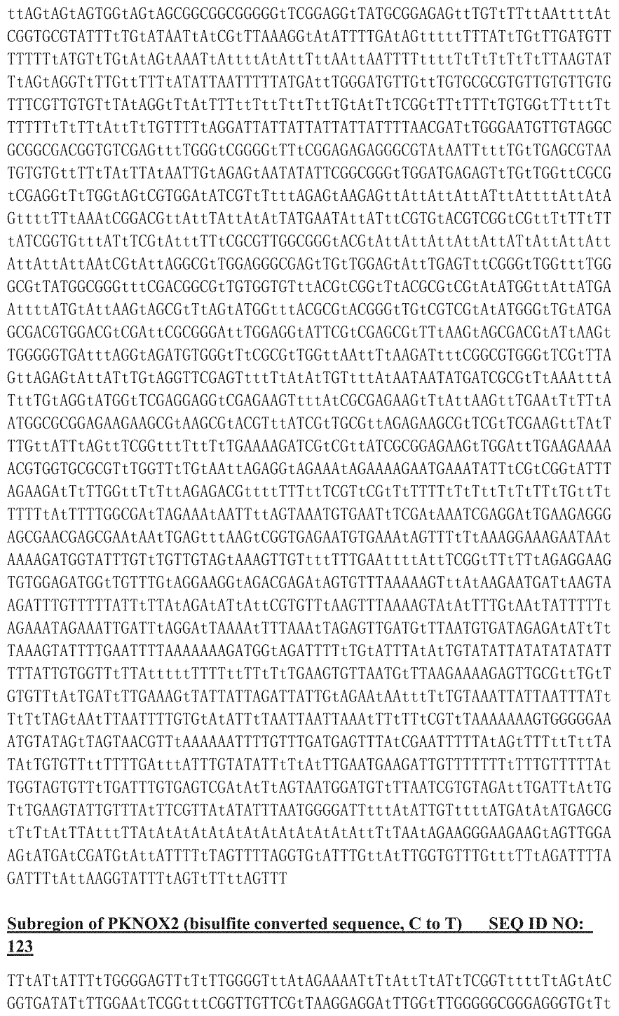

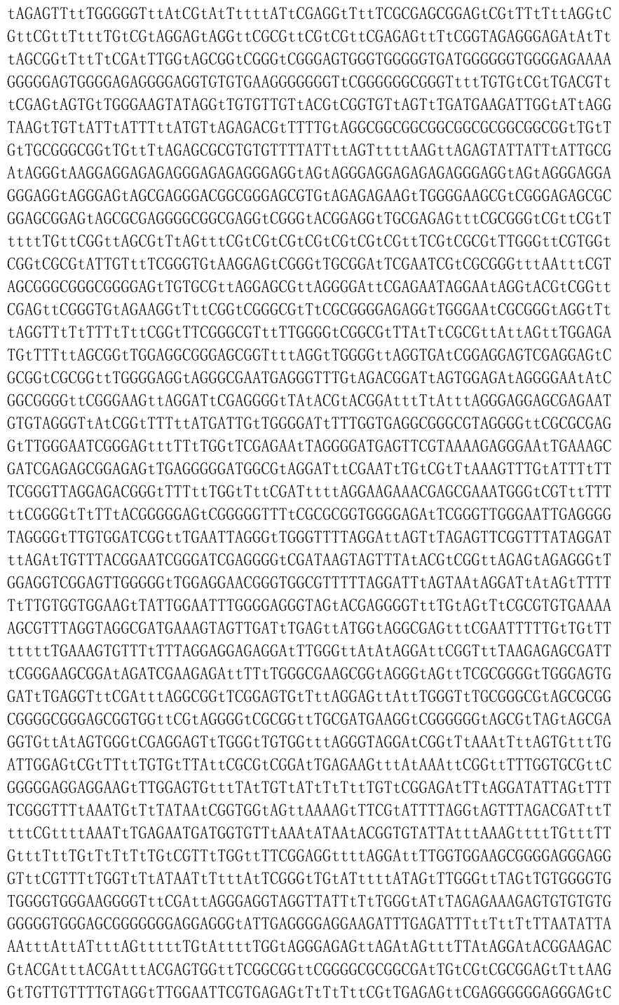

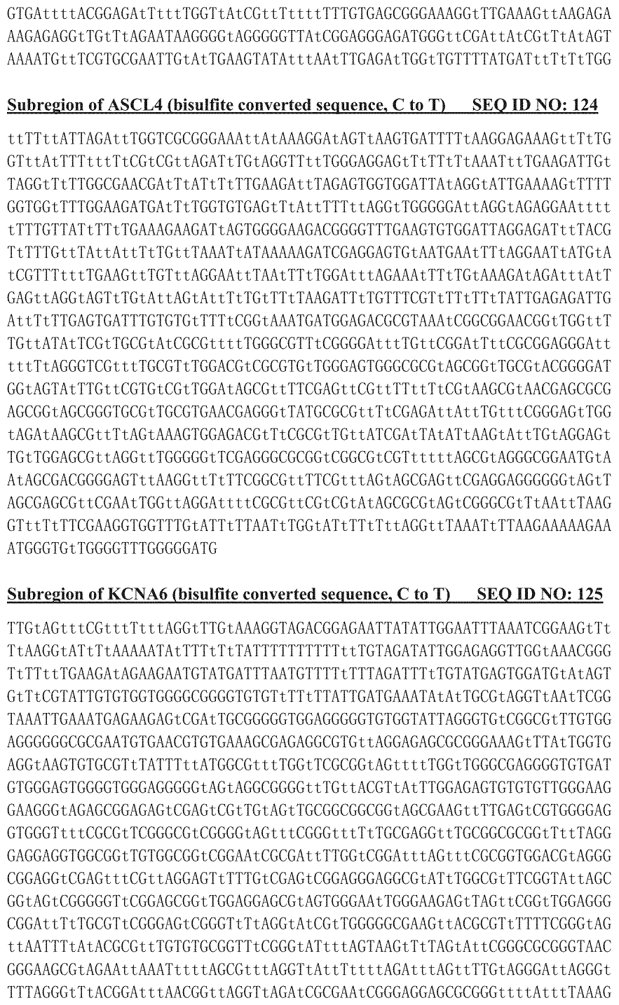

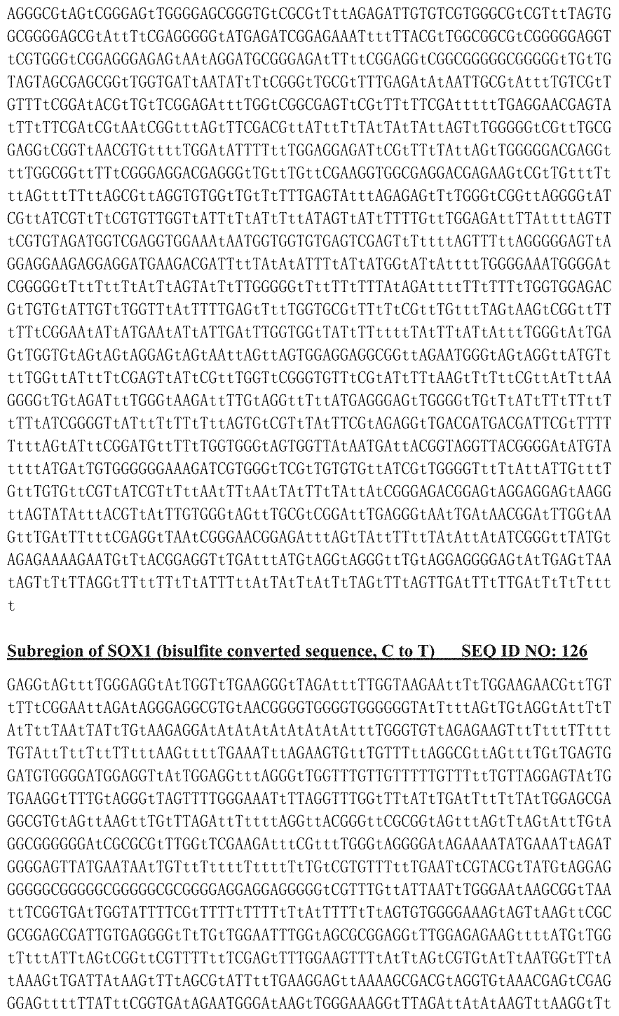

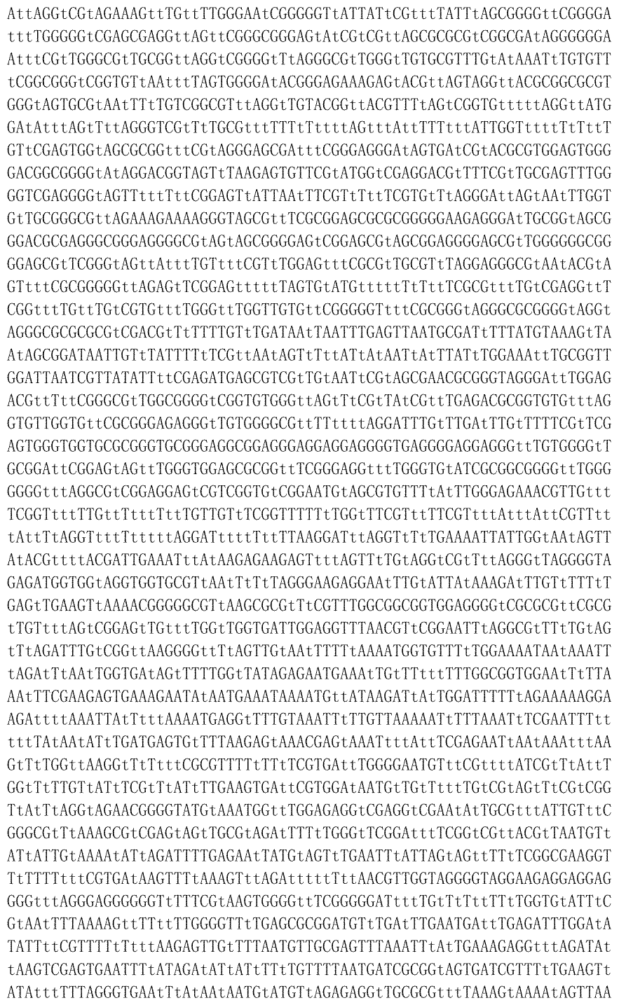

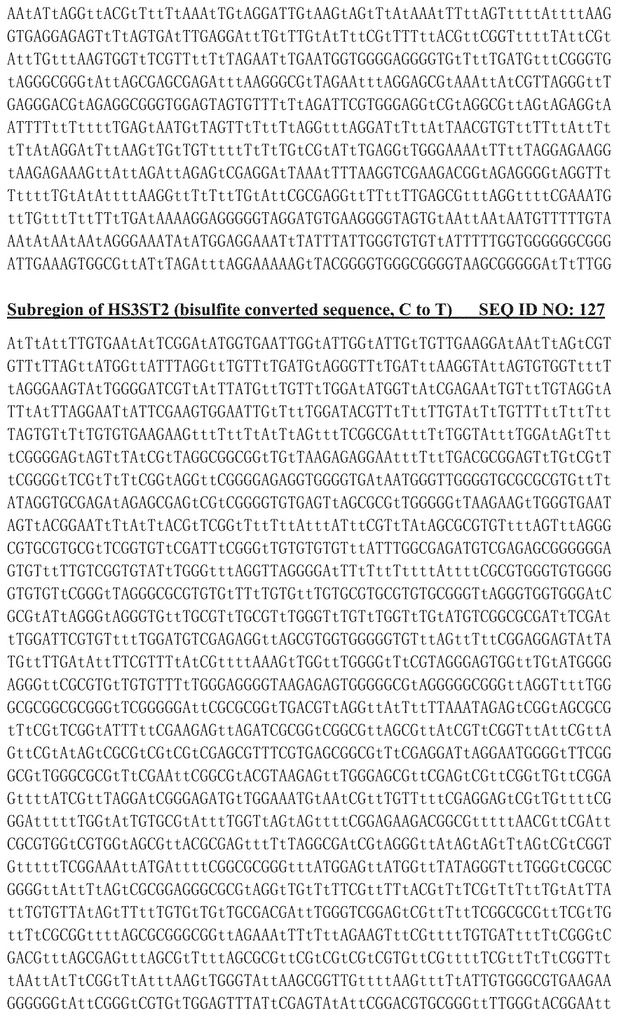

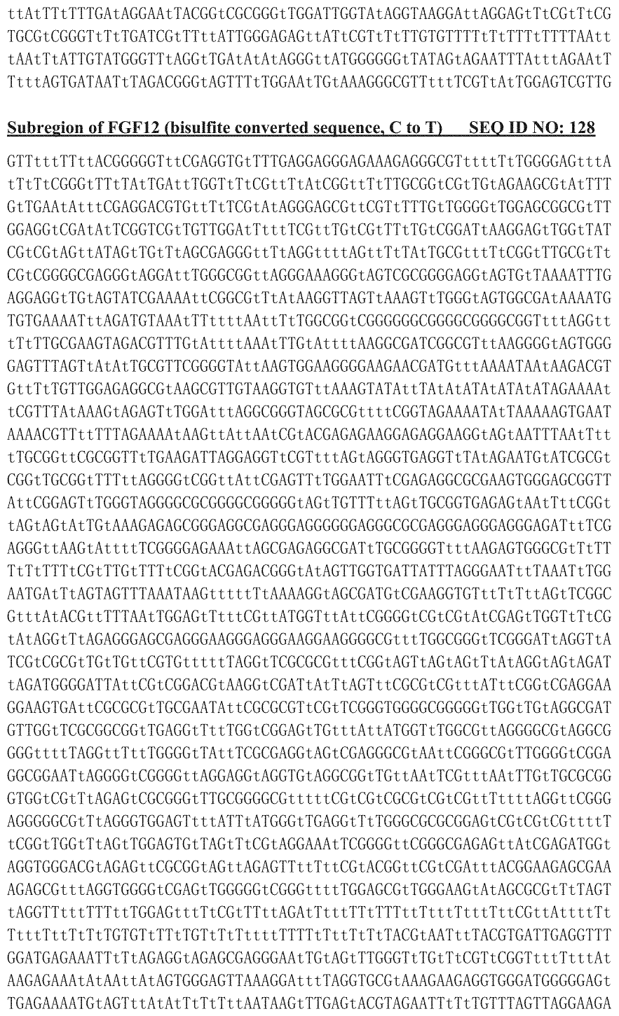

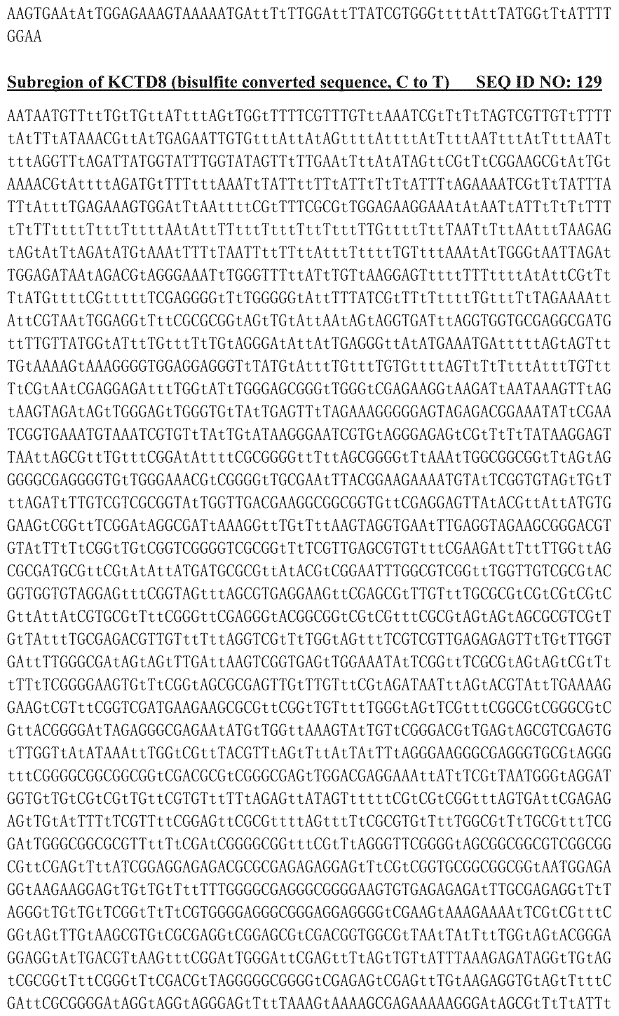

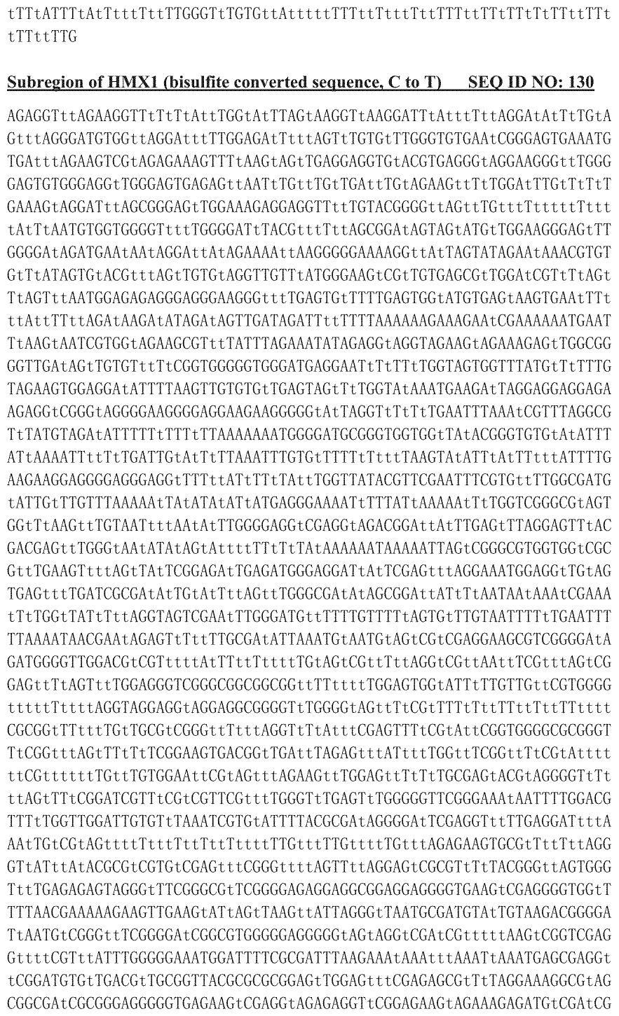

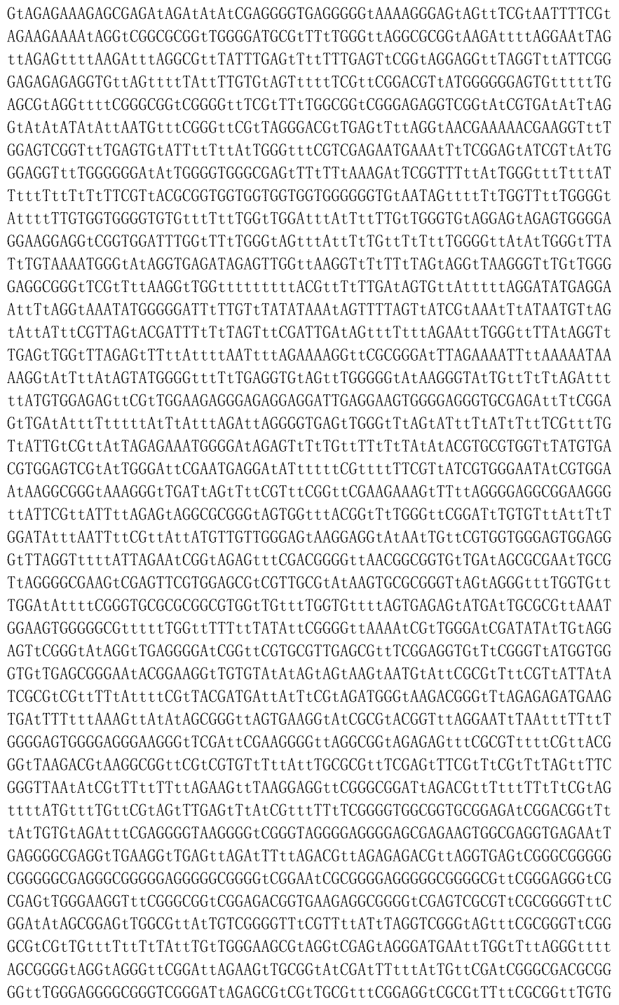

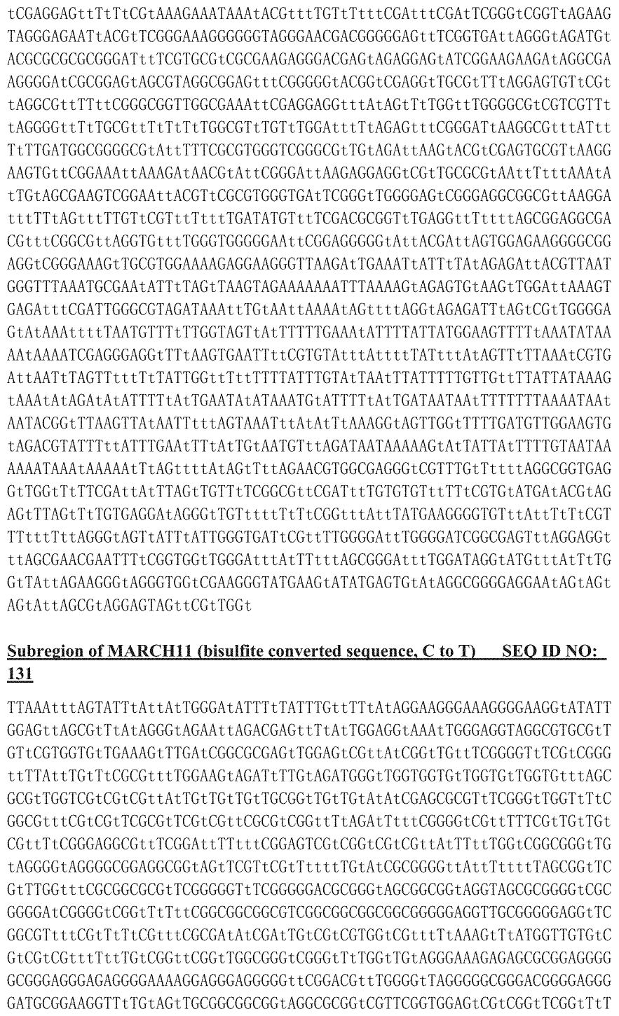

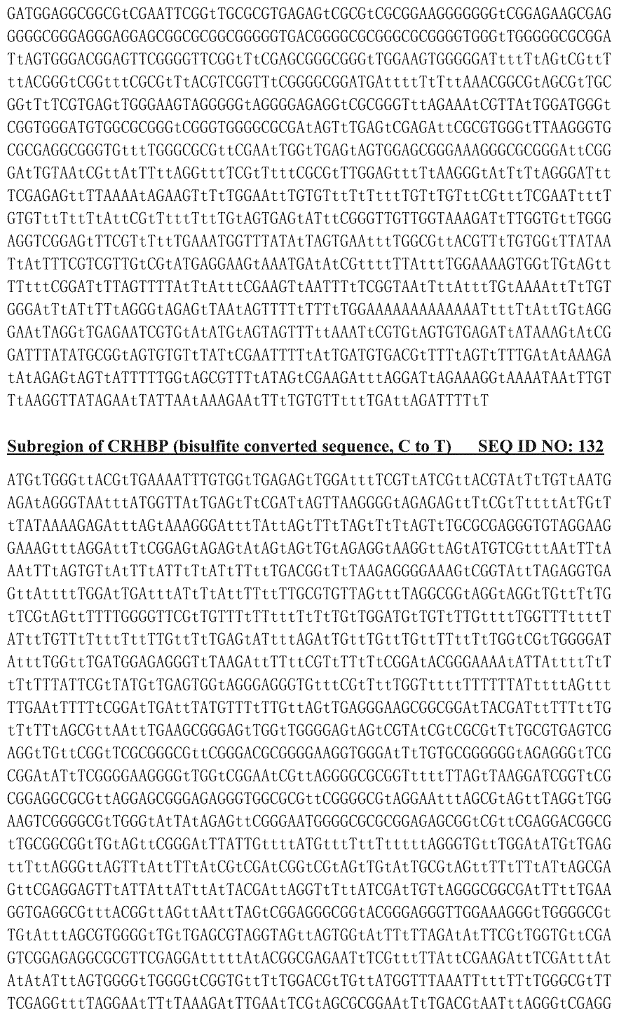

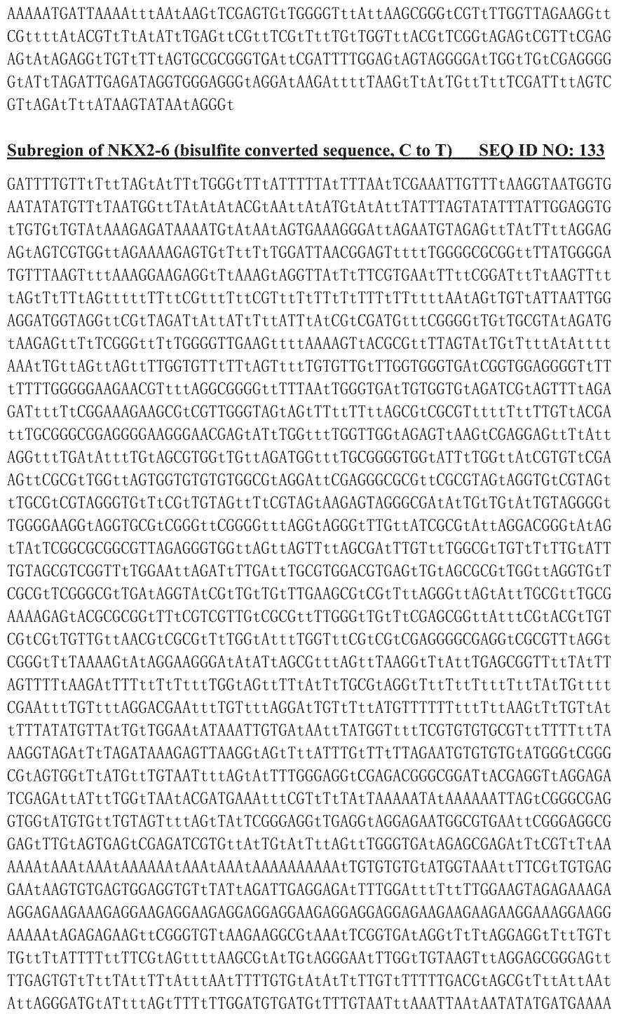

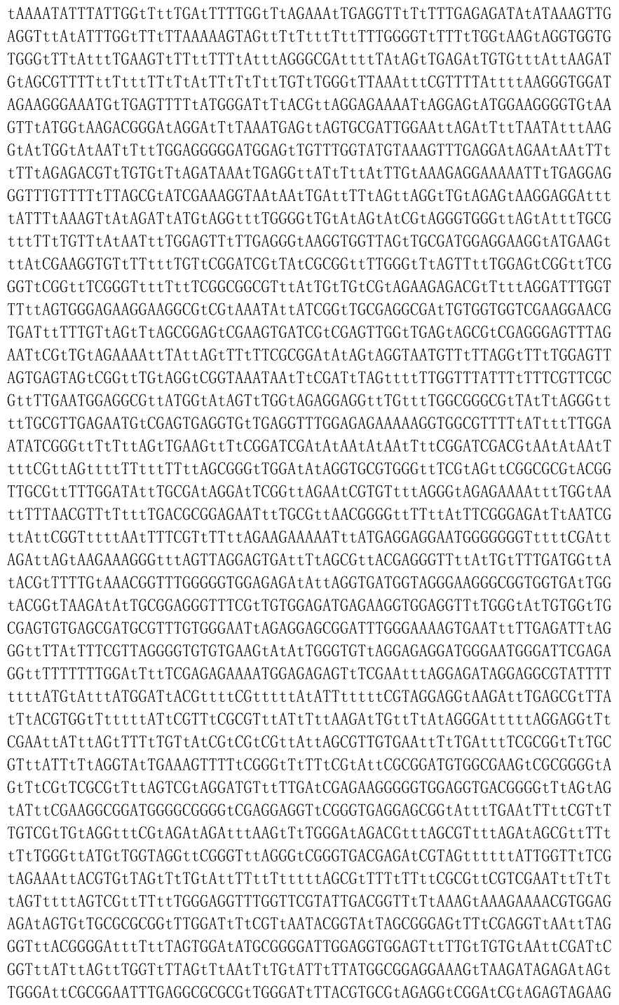

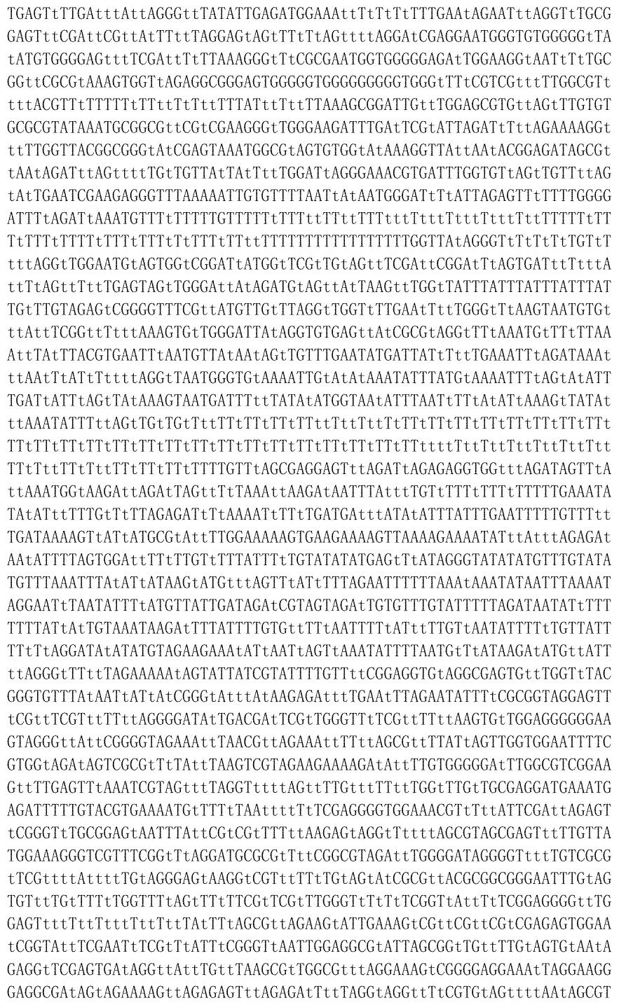

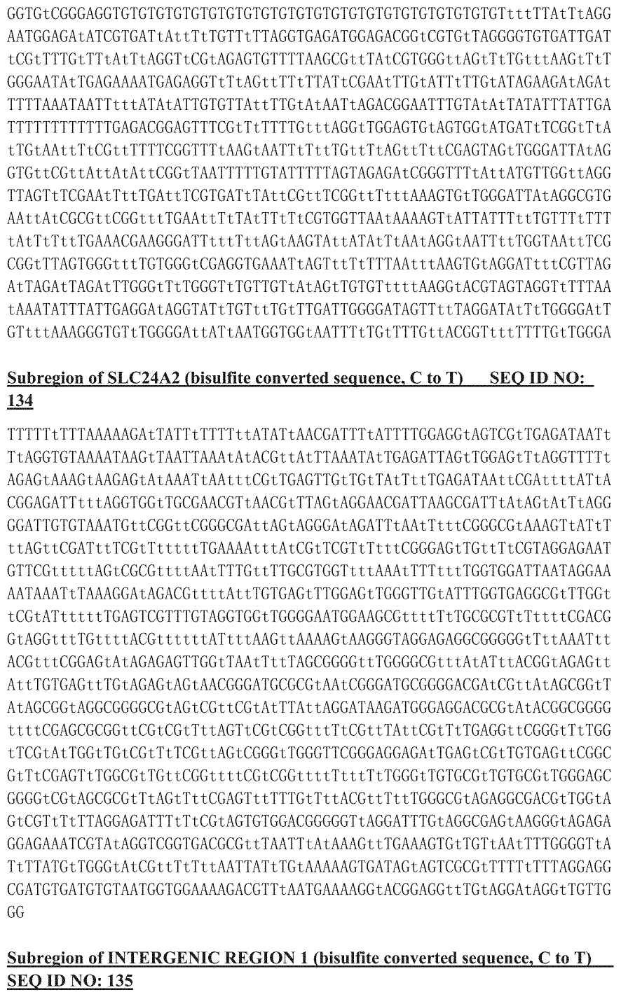

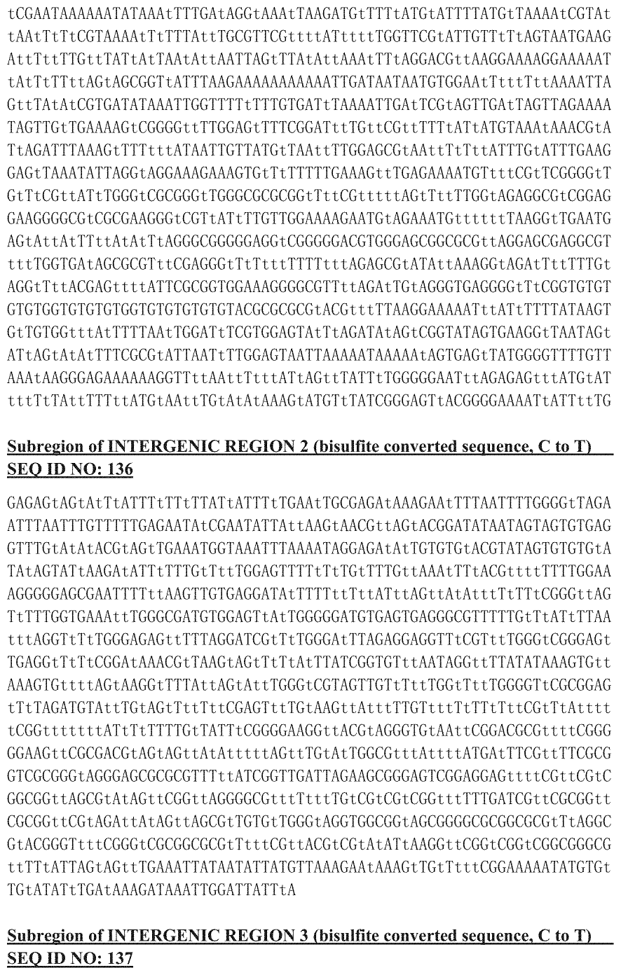

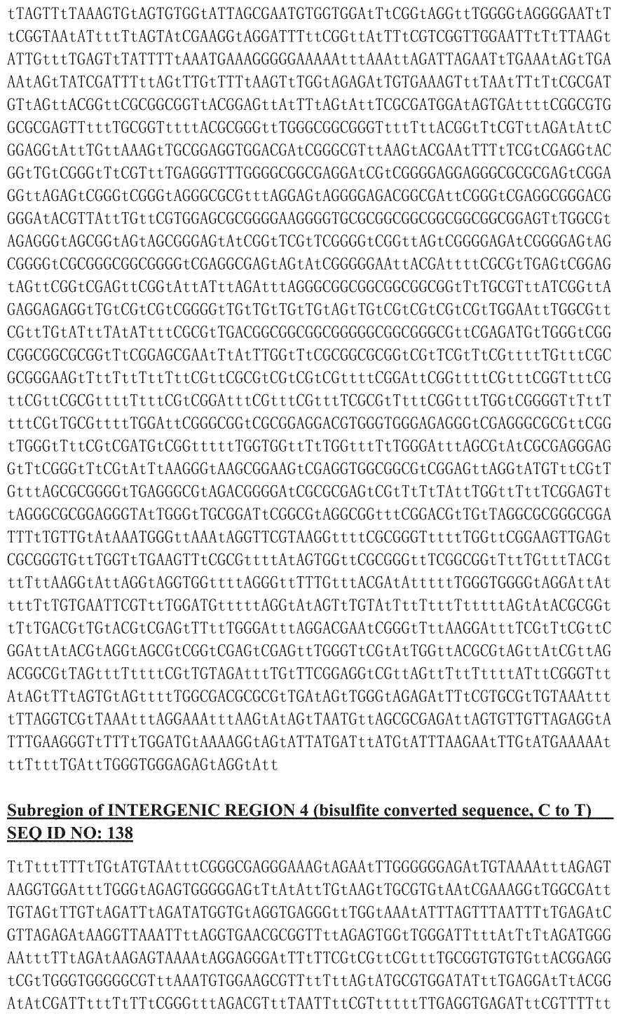

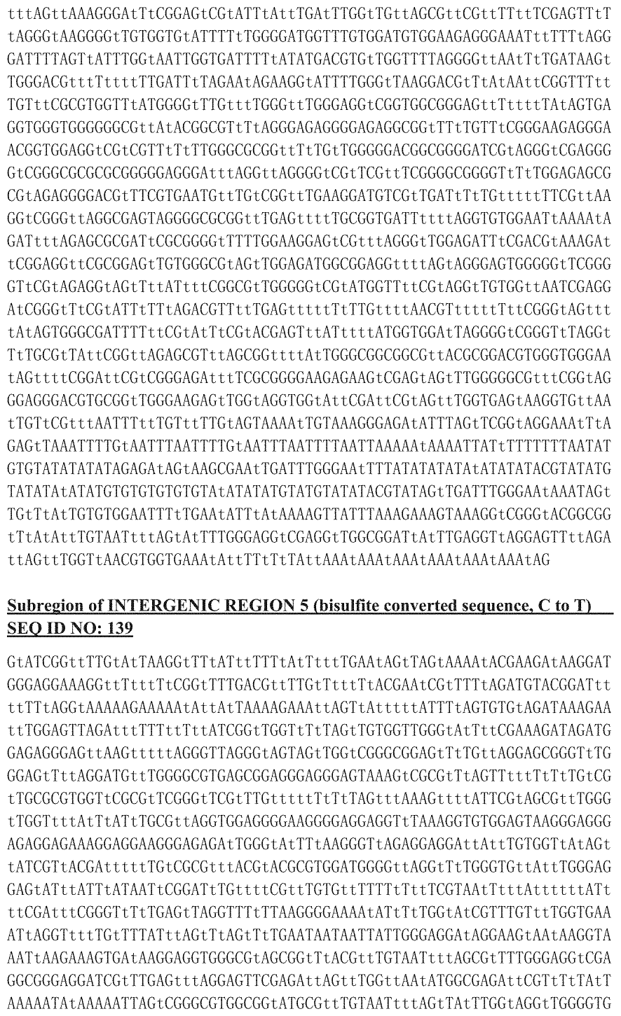

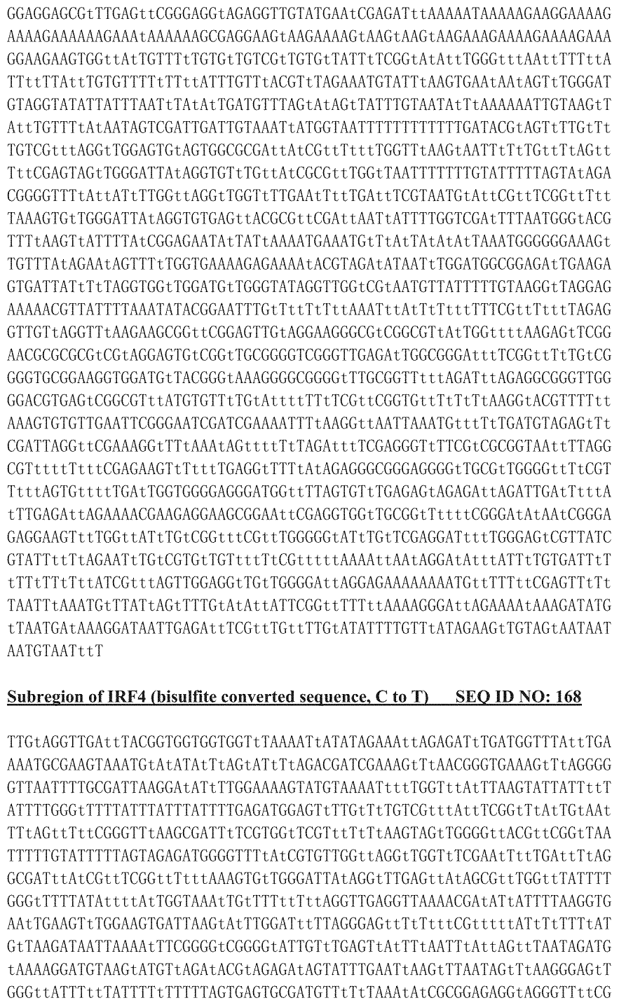

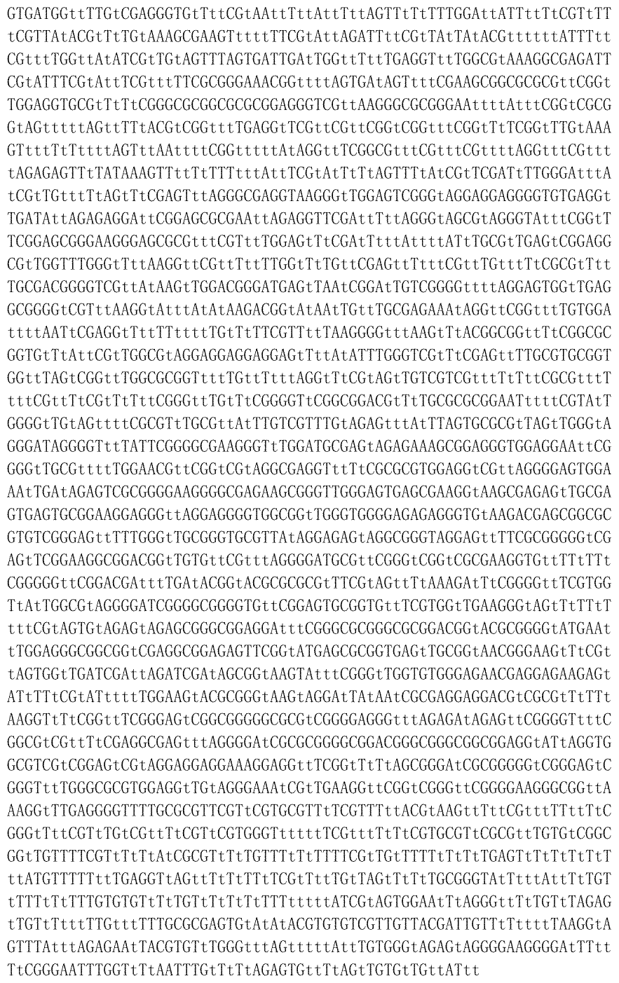

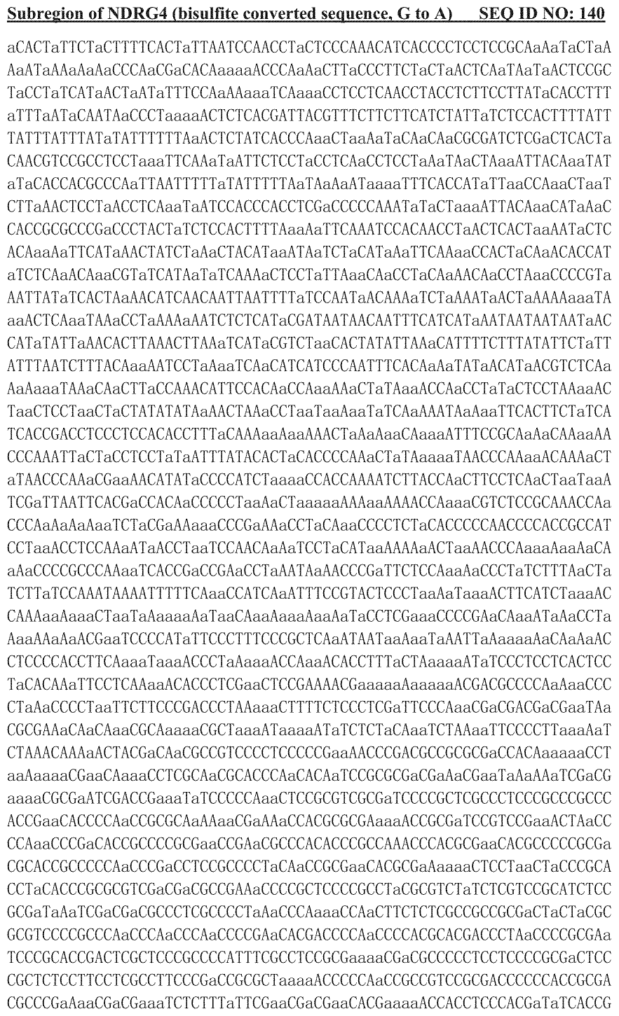

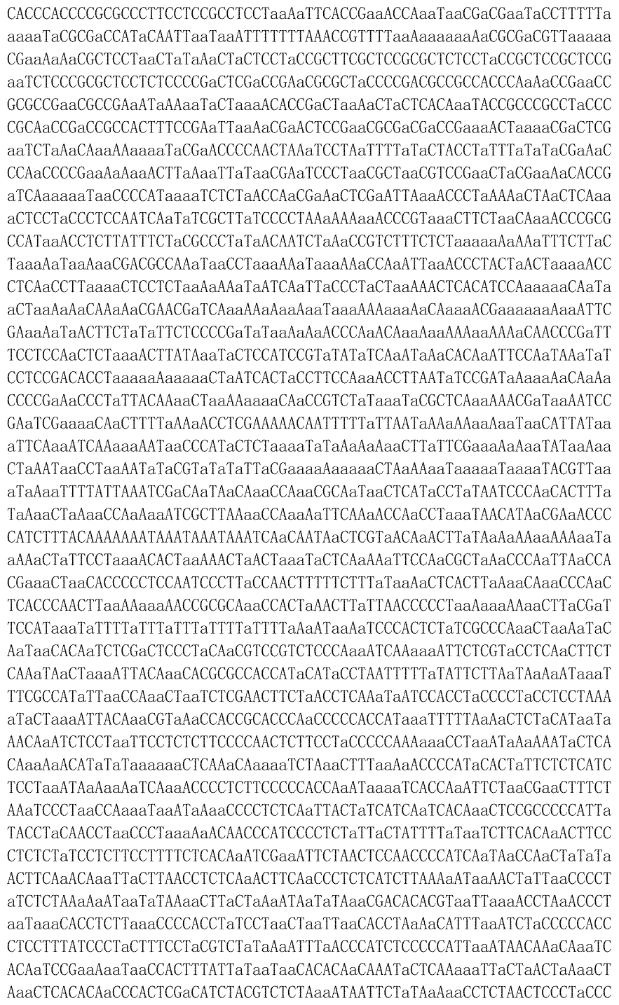

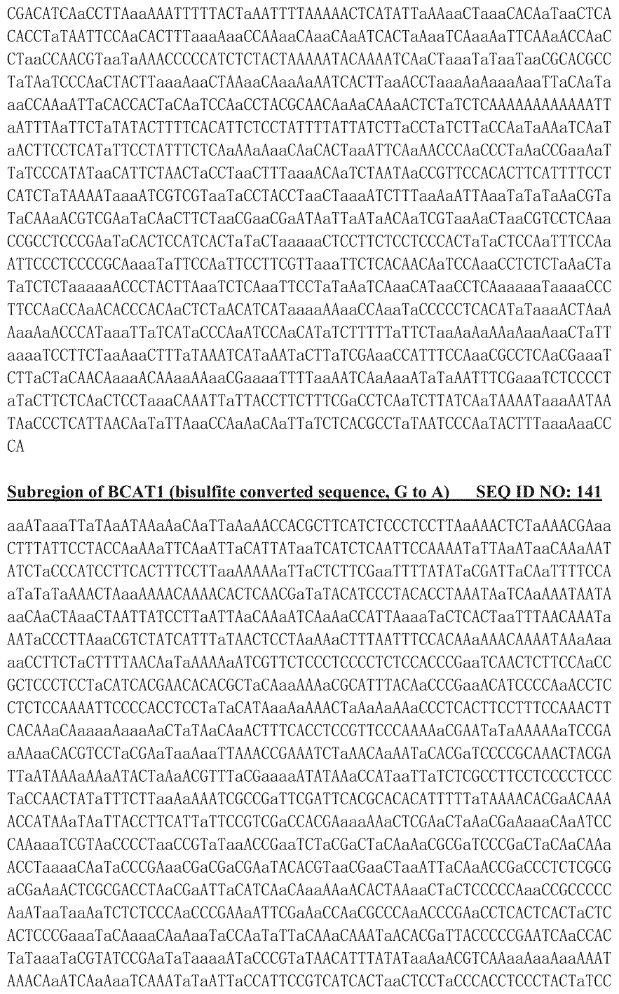

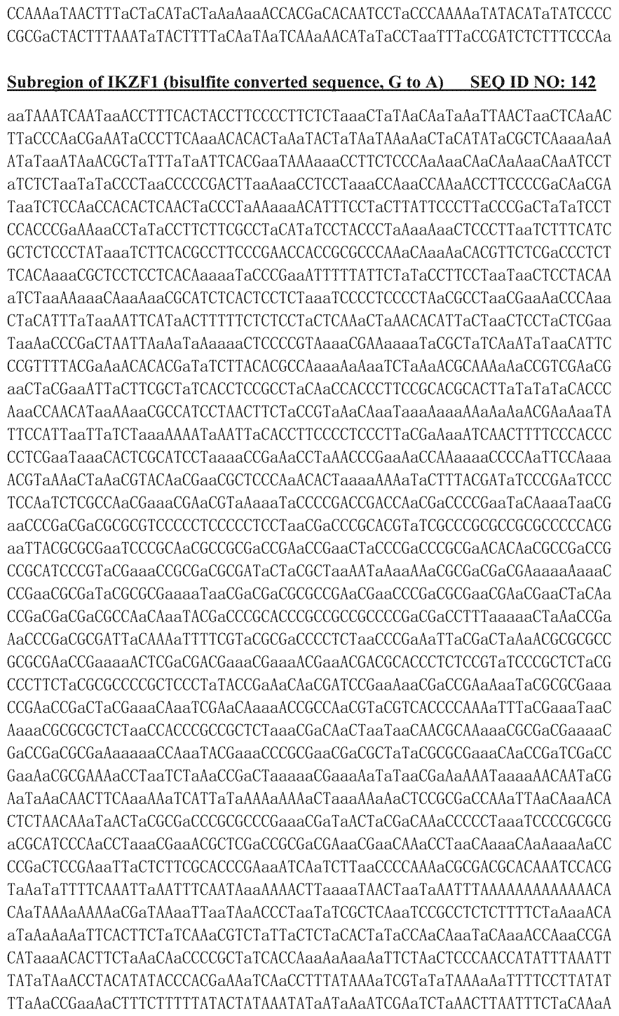

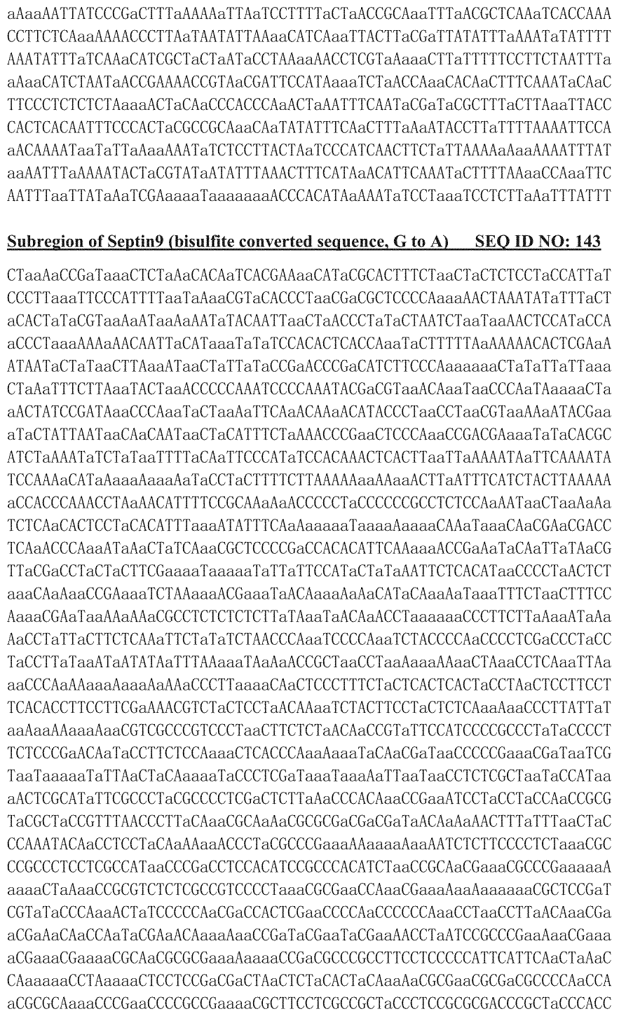

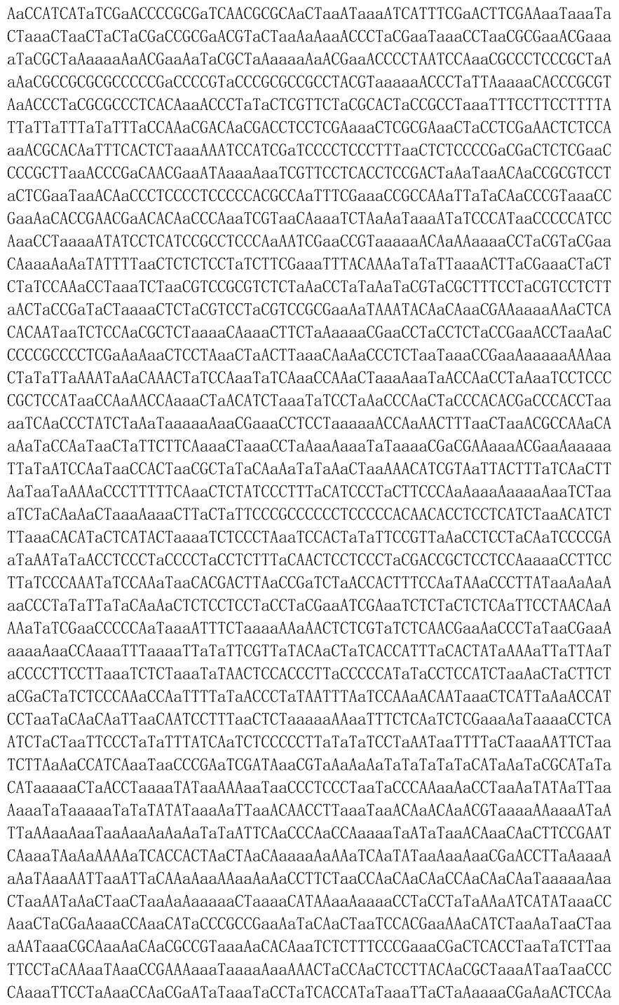

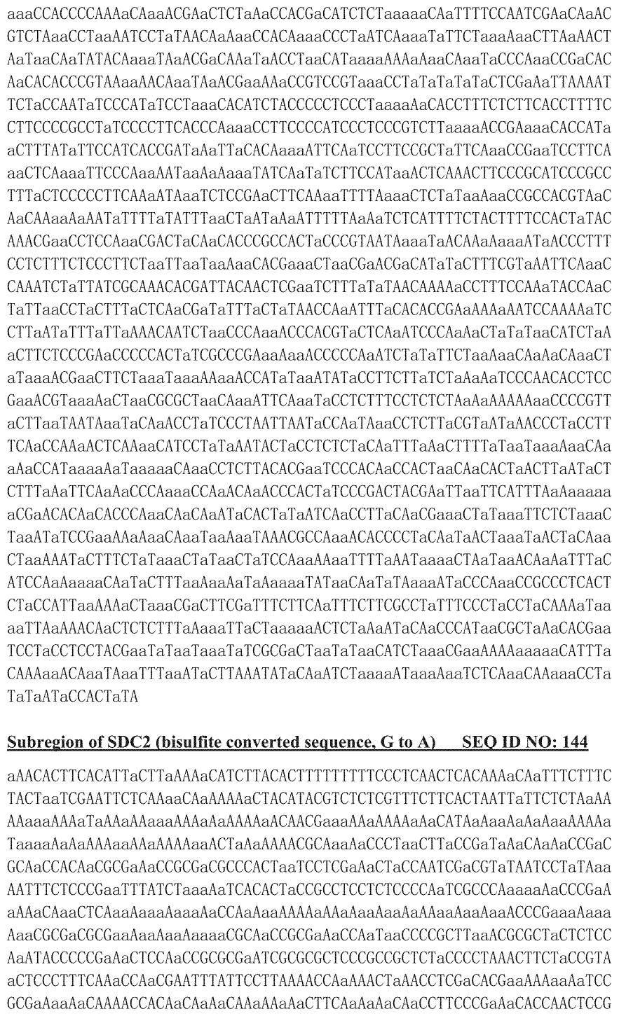

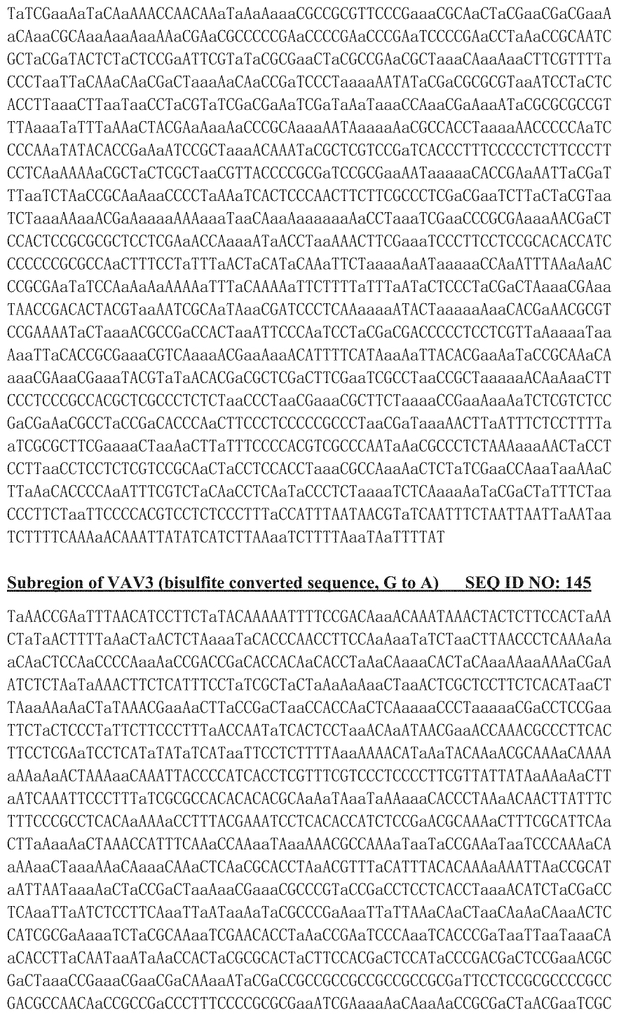

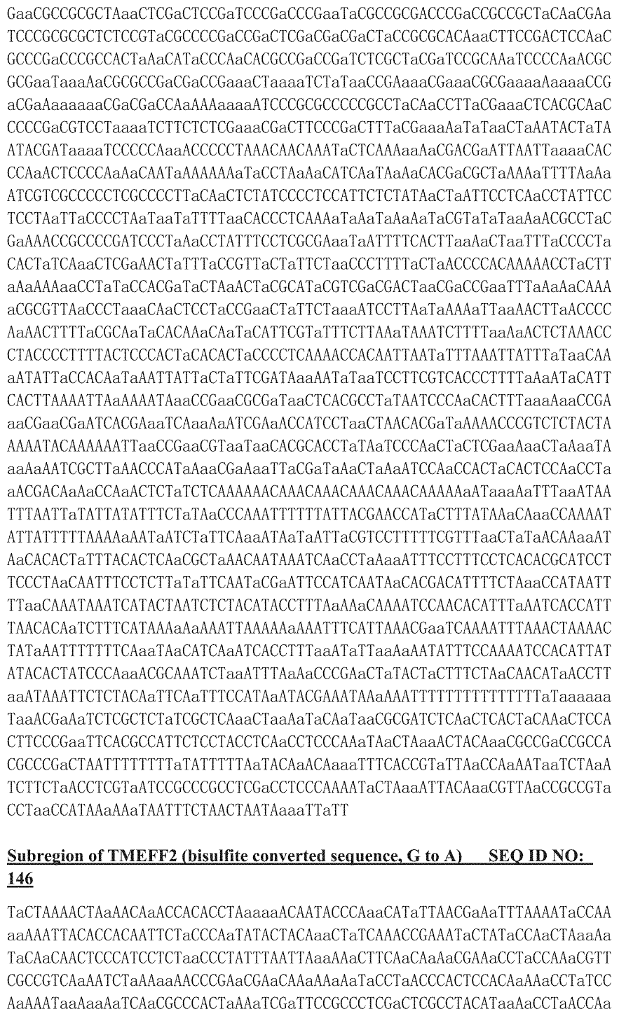

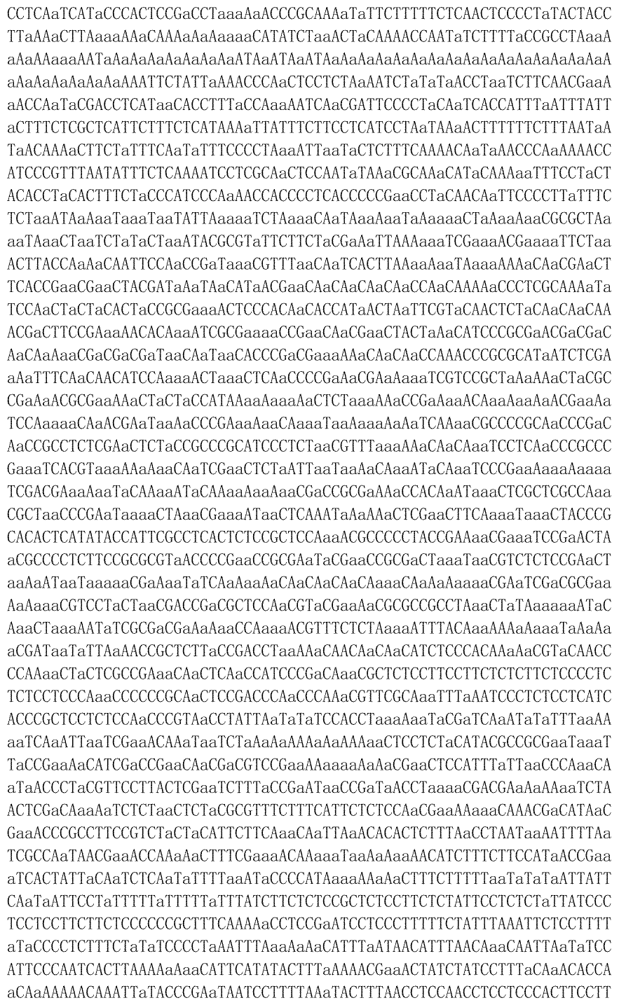

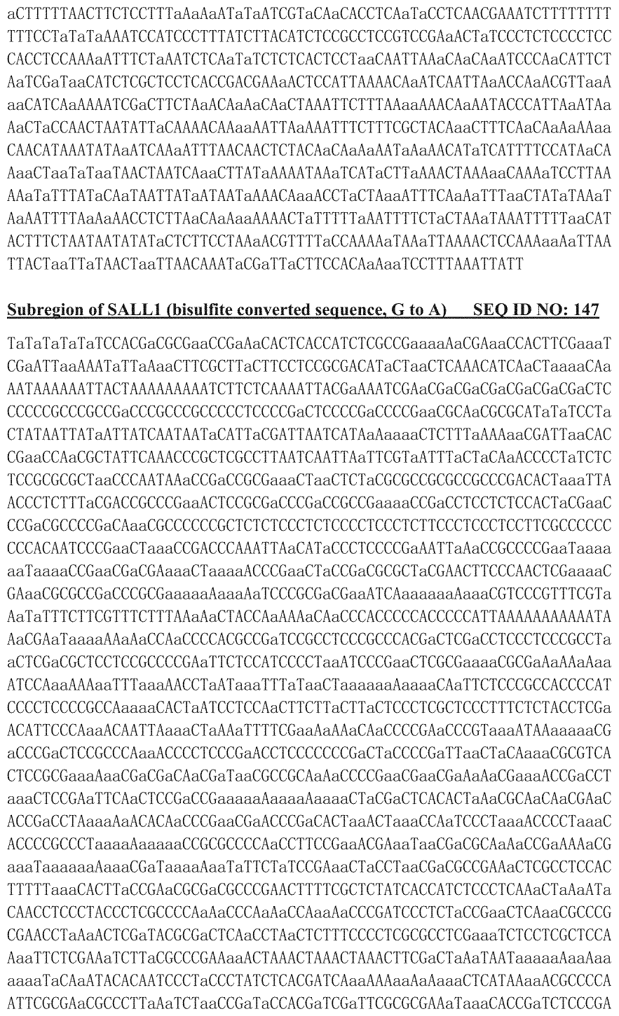

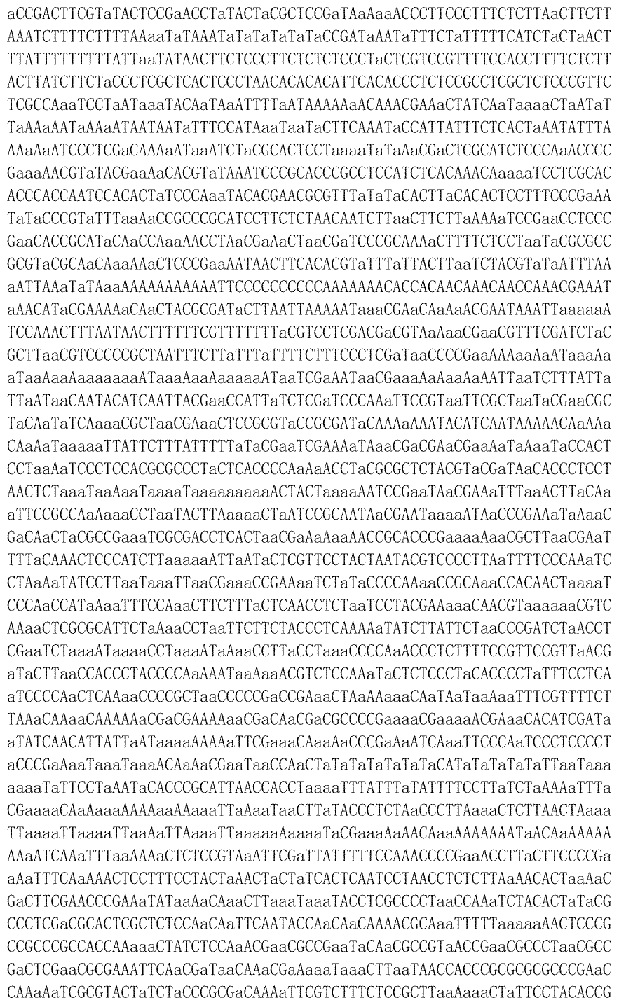

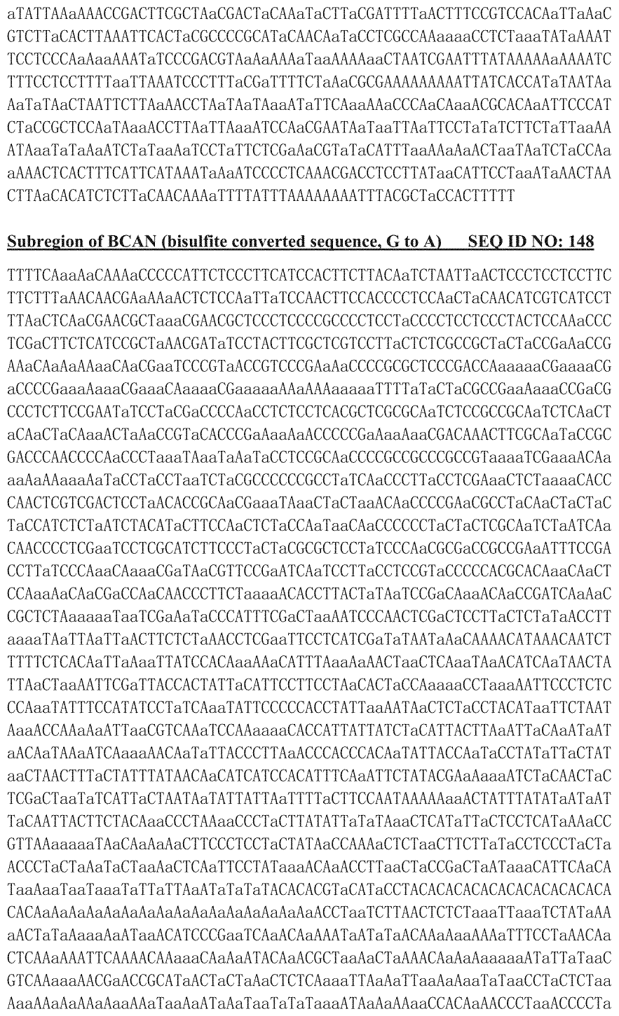

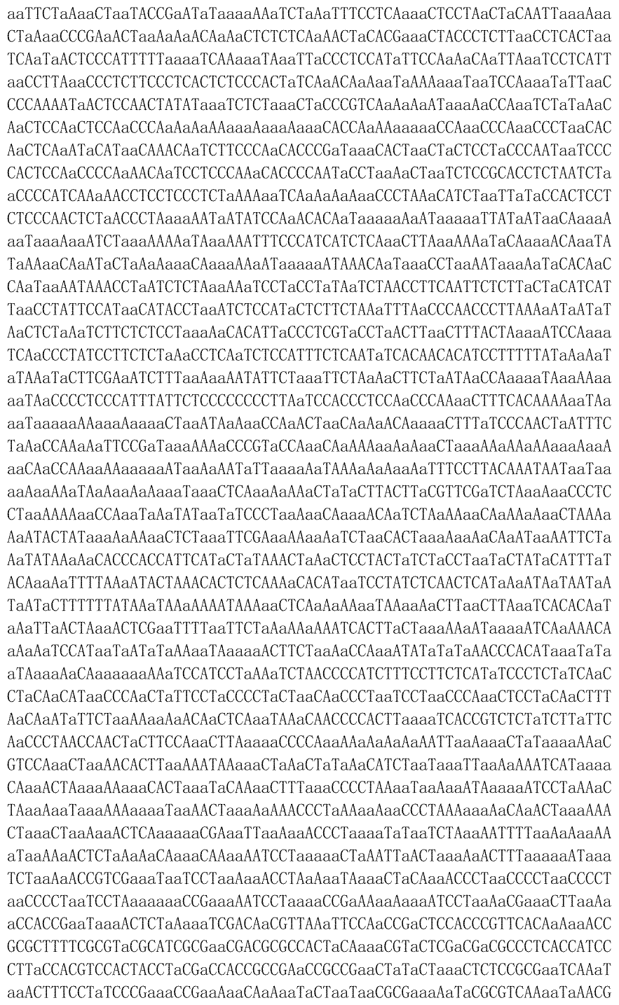

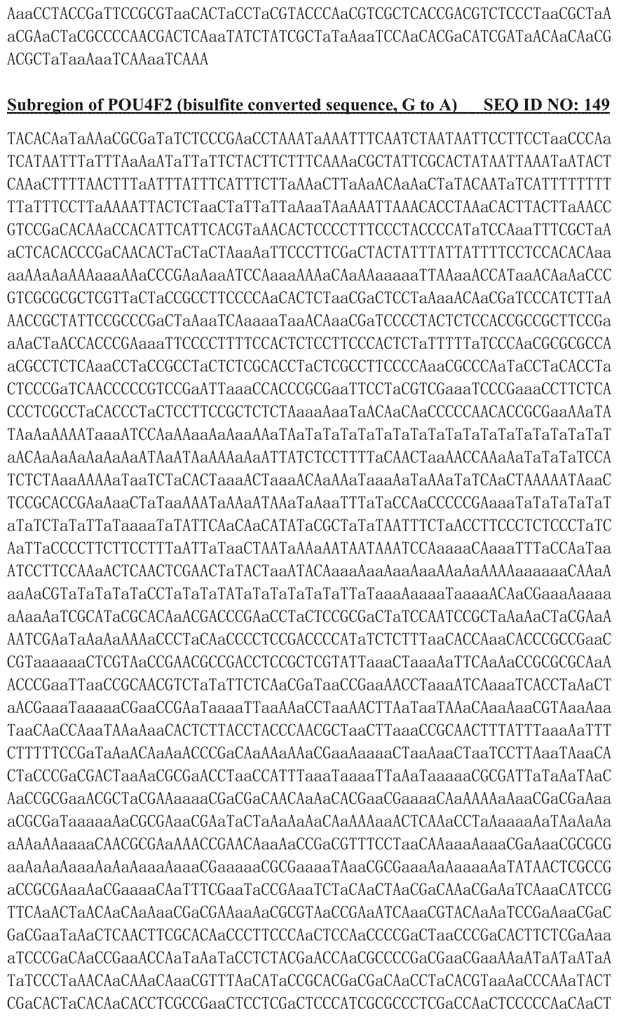

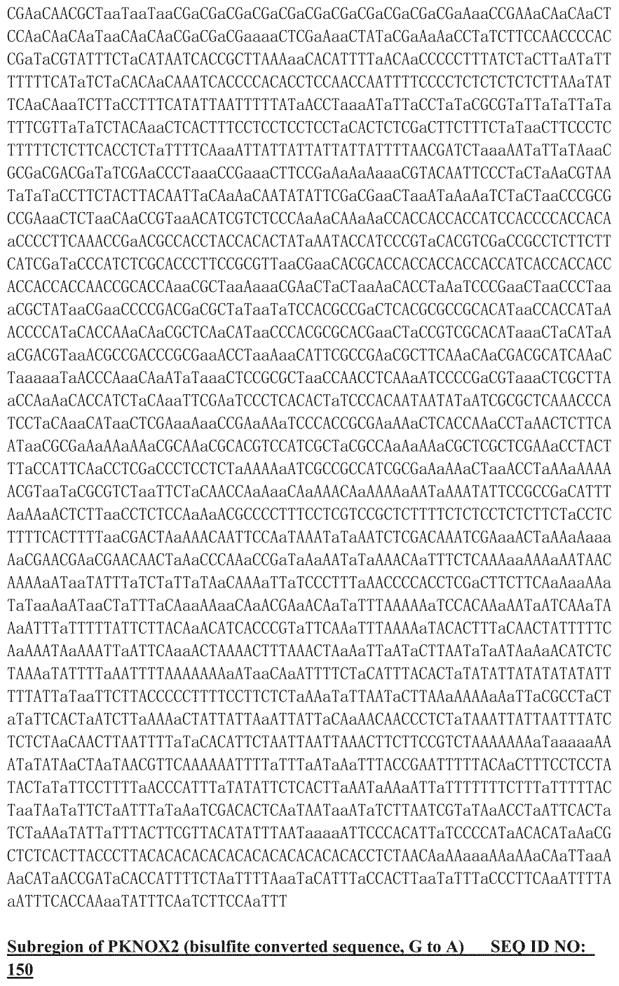

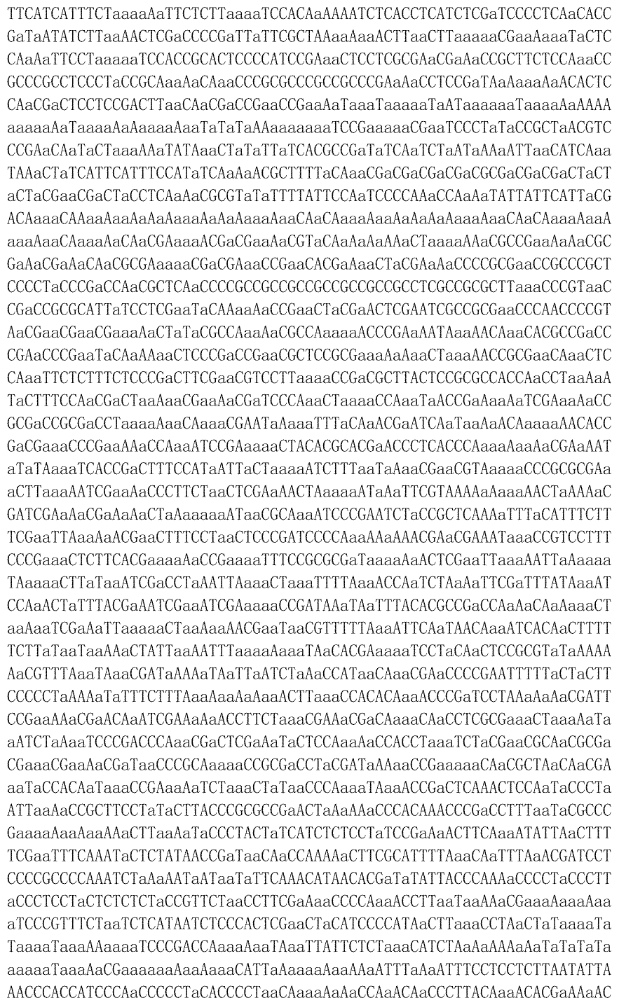

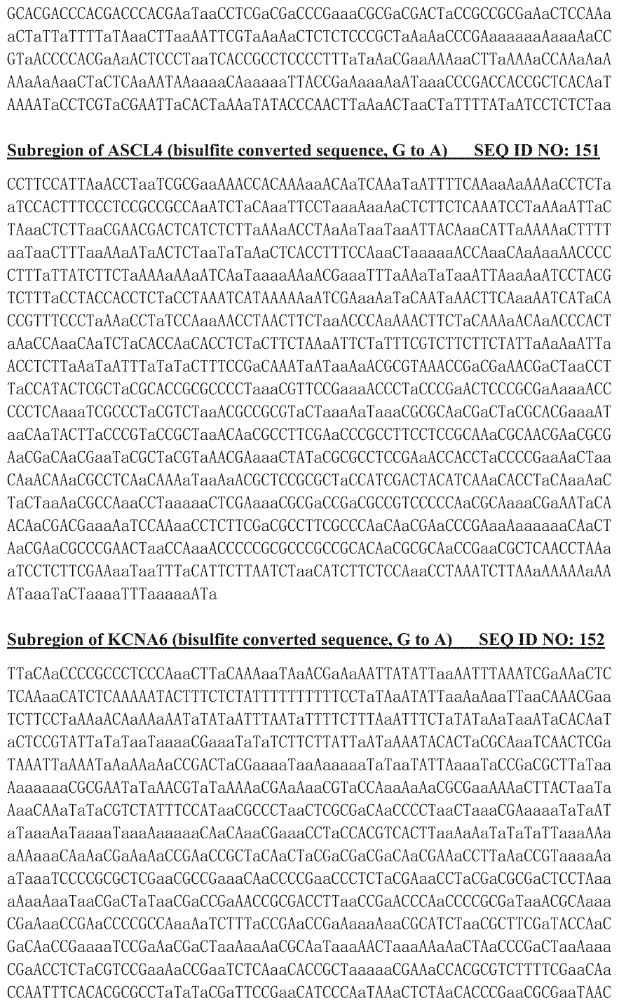

The Ct value of each tested target marker was quantified to identify its presence or absence of the methylated copies in CRC samples. Alternatively, delta Ct value of each tested target marker to the internal control ACTB can be calculated to represent the relative methylation level. Importantly, all tested markers had classification power to separate CRC from controls with an AUC ranging from 0.8 to 0.9 (as shown in FIG. 4). Different algorithms, such as Linear Discriminant Analysis, SVM, Random forest, Linear Regression, Logistic regression etc. have been used to build a classifier of early cancer detection. Different combinations of markers have been used to achieve the optimized performance. The ROC curve for one of the combinations (SALL1, BCAT1, and Septin9) was shown in FIG. 5. The ROC curves for the other combinations were similar to FIG. 5, and were not shown here.

### Example 4: LOD Comparison Between Pre-Amplification Method and Direct qPCR Method

To compare the LOD of Pre-Amplification Method and Direct qPCR Method, the inventors tested 13 target markers (i.e. VAV3, NDRG4, Septin9, BCAT1, IKZF1, BCAN, POU4F2, SALL1, PKNOX2, SDC2, ASCL4, TMEFF2 and INTERGENIC REGION 1) both with Pre-Amplification Method and Direct qPCR Method. The Direct qPCR Method was performed the same as the Pre-Amplification Method, except that there is not a pre-amplification step. In each method, the 13 target markers were pre-amplified/amplified simultaneously, but the quantification was performed separately for each target marker. The LOD comparisons between the Pre-Amplification Method and Direct qPCR Method for target marker VAV3 were shown below. The LOD comparisons between the Pre-Amplification Method and Direct qPCR Method for the other 12 target markers were carried out similarly, and were not shown here.

Briefly, CRC tissue DNA was spiked into blood cell DNA with 0.5% and 0.2% ratio, 40 ng DNA was bisulfite-treated (MethylCode™ Bisulfite Conversion Kit), wherein half converted DNA was used for pre-amplification and then qPCR (i.e. the Pre-Amplification Method), and the other half converted DNA was used for qPCR directly (i.e. the Direct qPCR Method). Final primer concentration in the pre-amplification step was 50 nM. The 25 μL PCR mix was composed of 10 μL of the converted DNA, 2.5 μL of premixed solution containing the primers set forth above; and 12.5 μL of PCR reagent mix (Luna® Universal Probe qPCR Master Mix (NEB)). The PCR program was 3 mins at 95° C., followed by 8 cycles of 30 seconds at 95° C. and 60 seconds at 56° C. The achieved product after the pre-amplification step was diluted in 10 folds and used for qPCR. The qPCR mix was composed of 10 μL template DNA, 2.5 μL primers/probe pool and 12.5 μL of LUNA master mix. The qPCR program was 5 mins at 95° C., followed by 50 cycles of 15 seconds at 95° C. and 40 seconds at 56° C. (during which fluorescence was detected), run on ABI 7500 Real-Time PCR System. 4 replicates were done in parallel. The results were shown Table 5 below.

TABLE 5
Comparison of the results between the Pre-
Amplification Method and Direct qPCR Method

| | Pre-Amplification | | | |
|---|---|---|---|---|
| CRC DNA | Method | | Direct qPCR Method | |
| percentage | Ct value | Mean ± SD | Ct value | Mean ± SD |
| 0.50% | 25.08 | 24.91 ± 0.36 | 35.39 | 36.31 ± 1.11 |
| | 24.66 | | 37.07 | |
| | 25.32 | | 35.33 | |
| | 24.56 | | 37.45 | |
| 0.20% | 26.54 | 25.75 ± 0.58 | 39.58 | NA |
| | 25.67 | | Undetermined | |
| | 25.65 | | 43.37 | |
| | 25.12 | | Undetermined | |

As shown in Table 5, compared with the Direct qPCR Method, the Pre-Amplification Method showed improved LOD (0.50% vs. 0.20% CRC DNA percentage), stability, and higher detect sensitivity. The Pre-Amplification Method for the other 12 target markers (i.e. NDRG4, Septin9, BCAT1, IKZF1, BCAN, POU4F2, SALL1, PKNOX2, SDC2, ASCL4, TMEFF2 and INTERGENIC REGION 1) shown better or not worse results than Direct qPCR Method, and the results were not shown here.

### Example 5: Quantification of Methylated Target Markers Using Cell-Free DNA, and Comparison to No Pre-Amplification Method

To validate the clinical performance of methylated markers to CRC plasma samples, we tested 5 markers (Septin9, BCAT1, IKZF1, BCAN, VAV3) in 32 clinically diagnosed CRC plasma samples and 29 plasma control samples negative in colonoscopy using both Pre-Amplification Method and No Pre-Amplification Method. The No Pre-Amplification Method was performed similar to the Pre-Amplification Method, except that the pre-amplification step and dilution step are absent. Among the 32 clinically diagnosed CRC plasma samples, 2 samples were from subjects diagnosed as in CRC Stage I, 9 samples were from subjects diagnosed as in CRC Stage II, 13 samples were from subjects diagnosed as in CRC Stage III, and 5 samples were from subjects diagnosed as in CRC Stage IV, 3 samples were stage unknown.

The experiments included the following steps:

1. Cell-free DNA (cfDNA) samples were obtained from 3-5 ml plasma samples by using QIAamp Circulating Nucleic Acid Kit (Qiagen).

2. If the DNA was less than 40 ng, cfDNA was divided to two parts and used as the input for bisulfite conversion with a bisulfite reagent (MethylCode™ Bisulfite Conversion Kit) to obtain converted cfDNA in two parallel reactions, one with 10 μL elution for pre-amplification method, the other one with 20 μL elution. If DNA more than 40 ng, both 20 ng cfDNA was used for two reactions, and the elution procedure was the same as above.

3. For the Pre-Amplification Method, the converted cfDNA samples in one reaction (10 μL elution) were pre-amplified. Briefly, the converted cfDNA samples obtained from step 2 above were pre-amplified by PCR reactions in the presence of methylation-specific primer pairs (see Table 2), specific for Septin9, BCAT1, IKZF1, BCAN, VAV3. In the PCR reaction system, each of the primers was at a final concentration of 200 nM. The pre-amplification program, dilution and qPCR assays were the same as Example 3.

4. For the No Pre-Amplification Method, the converted cfDNA samples in the other reaction (20 μL elution) were used for qPCR assays in two different wells, each well with 10 μL converted DNA. The qPCR mix and program were the same as Pre-Amplification Method.

5. Non-CpG ACTB region was used as internal control for each reaction well (see Table 2). The detection probes for different markers were labeled with different fluorescence. In the PCR reaction system, each of the primers was at a final concentration of 500 nM, and each of the detection probes was at a final concentration of 200 nM.

Results

The Ct value was set as 50 for a sample without amplification signal. A reference Ct value was set for each tested marker, respectively. If the Ct value of any one of the tested markers is identical or lower than its corresponding reference Ct value, then the sample would be classified as a positive sample.

Table 6 below shows the comparison results by using 5 target markers (Septin9, BCAT1, IKZF1, BCAN and VAV3) in the Pre-Amplification Method and No Pre-Amplification Method. As shown in Table 6, the Pre-Amplification Method showed ultra high sensitivity (96.9%) for CRC and high specificity (93.1%) for population negative in colonoscopy, the sensitivity and specificity for No Pre-Amplification Method were 84.4% and 93.1%, respectively. The sensitivity ofNo Pre-Amplification Method was also much higher than Septin9 Alone Method.

TABLE 6
Comparison of the results between the Pre-Amplification
Method and No Pre-Amplification Method.

| | Pre-Amplification | | No Pre-Amplification | |
|---|---|---|---|---|
| | Method | | Method | |
| | | Number of | | Number of |
| Clinical | | Positive | | Positive |
| Classification | Accuracy | Samples | Accuracy | Samples |
| Colorectal | 96.9% | 31 | 84.4% | 27 |
| Cancer | | | | |
| Negative in | 93.1% | 2 | 93.1% | 2 |
| colonoscopy | | | | |

To validate the clinical performance of methylated markers to CRC plasma samples, we test more markers, including any combination of the markers selected from the group consisting of Septin9, BCAT1, IKZF1, BCAN, VAV3, NDRG4, and IRF4 in clinically diagnosed CRC plasma samples and plasma control samples negative in colonoscopy using both Pre-Amplification Method and No Pre-Amplification Method as described above. For example, any one of following combinations is tested: (1) Septin9, (2) Septin9, BCAT1; (3) Septin9 and IKZF1; (4) Septin9 and NDRG4; (5) Septin9 and BCAN; (6) Septin9 and VAV3; (7) Septin9 and IRF4; (8) BCAT1 and IKZF1; (9) BCAT1 and NDRG4; (10) BCAT1 and BCAN; (11) BCAT1 and VAV3; (12) BCAT1 and IRF4; (13) IKZF1 and NDRG4; (14) IKZF1 and BCAN; (15) IKZF1 and VAV3; (16) IKZF1 and IRF4; (17) NDRG4 and BCAN; (18) NDRG4 and VAV3; (19) NDRG4 and IRF4; (20) BCAN and VAV3; (21) BCAN and IRF4; (22) VAV3 and IRF4; (23) Septin9, BCAT1, and IKZF1; (24) BCAT1, IKZF1, and NDRG4; (25) IKZF1, NDRG4, and BCAN; (26) NDRG4, BCAN, and VAV3; (27) BCAN, VAV3, and IRF4; (28) Septin9, BCAT1, and NDRG4; (29) Septin9, BCAT1, and BCAN; (30) Septin9, BCAT1, and VAV3; (31) Septin9, BCAT1, and IRF4; (32) BCAT1, IKZF1, and BCAN; (33) BCAT1, IKZF1, and VAV3; (34) BCAT1, IKZF1, and IRF4.

### Example 6: CRC Detection by Quantification of CRC Methylated Target Markers (Septin9, BCAT1, IKZF1, VAV3 and IRF4) with Cell-free DNA

To assess the clinical performance of more marker combinations of, we tested 5 markers (Septin9, BCAT1, IKZF1, VAV3 and IRF4) in 286 clinically diagnosed CRC plasma samples and 112 plasma control samples negative in colonoscopy using the methods disclosed herein (also referred to as Pre-Amplification Method). Among the 286 clinically diagnosed CRC plasma samples, 48 samples were from subjects diagnosed as in CRC Stage I, 113 samples were from subjects diagnosed as in CRC Stage II, 107 samples were from subjects diagnosed as in CRC Stage III, and 18 samples were from subjects diagnosed as in CRC Stage IV.

The experimental method was similar to Example 3.

Results

The Ct value was set as 50 for a sample without amplification signal. A reference Ct value was set for each tested marker, respectively. If the Ct value of any one of the tested markers is identical or lower than its corresponding reference Ct value, then the sample would be classified as a positive sample.

As shown in Table 7, the Pre-Amplification Method (quantification of CRC methylated markers Septin9, BCAT1, IKZF1, VAV3 and IRF4) showed ultra high sensitivity (84.3%) for CRC and high specificity (90.3%) for population negative in colonoscopy.

TABLE 7
Comparison of the results between the Pre-Amplification
Method (methylated markers of Septin9, BCAT1,
IKZF1, VAV3 and IRF4) and colonoscopy

| Results of | | Number of | Number of | Total |
|---|---|---|---|---|
| Colonoscopy | Accuracy | Positive Samples | Negative Samples | Number |
| Colorectal | 84.3% | 241 | 45 | 286 |
| Cancer | | | | |
| Negative in | 90.3% | 11 | 101 | 112 |
| colonoscopy | | | | |

As shown in Table 8, in the Pre-Amplification Method (quantification of CRC methylated markers Septin9, BCAT1, IKZF1, VAV3 and IRF4), the sensitivity of CRC Stage I, Stage II, Stage III, and Stage IV was 62.5%, 85.8%, 88.8%, and 100%, respectively.

TABLE 8
The sensitivity in CRC detection of Pre-Amplification Method
when quantification of Septin9, BCAT1, IKZF1, VAV3 and IRF4.

| | | Number of | Number of | |
|---|---|---|---|---|
| Clinical | | Positive | Negative | Total |
| Classification | Accuracy | Samples | Samples | Number |
| Stage I | 62.5% | 30 | 18 | 48 |
| Stage II | 85.8% | 97 | 16 | 113 |
| Stage III | 88.8% | 95 | 12 | 107 |
| Stage IV | 100.0% | 18 | 0 | 18 |

To assess the clinical performance of more marker combinations of, we test more markers, including any combination of the markers selected from the group consisting of Septin9, BCAT1, IKZF1, BCAN, VAV3, NDRG4, and IRF4 in clinically diagnosed CRC plasma samples and plasma control samples negative in colonoscopy using the methods disclosed above. For example, any one of following combinations is tested: (1) Septin9, (2) Septin9, BCAT1; (3) Septin9 and IKZF1; (4) Septin9 and NDRG4; (5) Septin9 and BCAN; (6) Septin9 and VAV3; (7) Septin9 and IRF4; (8) BCAT1 and IKZF1; (9) BCAT1 and NDRG4; (10) BCAT1 and BCAN; (11) BCAT1 and VAV3; (12) BCAT1 and IRF4; (13) IKZF1 and NDRG4; (14) IKZF1 and BCAN; (15) IKZF1 and VAV3; (16) IKZF1 and IRF4; (17) NDRG4 and BCAN; (18) NDRG4 and VAV3; (19) NDRG4 and IRF4; (20) BCAN and VAV3; (21) BCAN and IRF4; (22) VAV3 and IRF4; (23) Septin9, BCAT1, and IKZF1; (24) BCAT1, IKZF1, and NDRG4; (25) IKZF1, NDRG4, and BCAN; (26) NDRG4, BCAN, and VAV3; (27) BCAN, VAV3, and IRF4; (28) Septin9, BCAT1, and NDRG4; (29) Septin9, BCAT1, and BCAN; (30) Septin9, BCAT1, and VAV3; (31) Septin9, BCAT1, and IRF4; (32) BCAT1, IKZF1, and BCAN; (33) BCAT1, IKZF1, and VAV3; (34) BCAT1, IKZF1, and IRF4.

1. A method of diagnosing colorectal neoplasm, screening for the onset or risk to the onset of colorectal neoplasm or assessing the development or prognosis of colorectal neoplasm in a subject, said method comprises the following steps:
(I). treating a DNA obtained from a biological sample with a reagent capable of distinguishing between an unmethylated site and a methylated site in the DNA, thereby obtaining a treated DNA;
(II). quantifying individual methylation level of a set of target markers within the treated DNA of step (I), wherein the target markers are selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(III). comparing the methylation level of at least one target marker of the set of target markers quantified at step (II) respectively with a corresponding reference level, wherein an identical or higher methylation level of one or more of the target markers relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

2. A method of diagnosing colorectal neoplasm, screening for the onset or risk to the onset of colorectal neoplasm or assessing the development or prognosis of colorectal neoplasm in a subject, said method comprises the following steps:
(I). treating a DNA obtained from a biological sample with a reagent capable of distinguishing between an unmethylated site and a methylated site in the DNA, thereby obtaining a treated DNA;
(II). quantifying individual methylation level of a set of target markers within the treated DNA of step (I), wherein at least two target markers are selected from the group consisting of Septin9, BCAT1, IKZF1, BCAN, PKNOX2, VAV3, NDRG4 and IRF4, and at least two target markers are selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP, and
(III). comparing the methylation level of at least one target marker of the set of target markers quantified at step (II) respectively with a corresponding reference level, wherein an identical or higher methylation level of one or more of the target markers relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

3. (canceled)

4. The method of claim 1, wherein the step (II) comprises:
(i) pre-amplifying at least a portion of at least one target marker of a set of target markers within the treated DNA obtained from step (I) with a pre-amplification primer pool, and the set of target markers are selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
(ii) quantifying individual methylation level of the set of target markers within achieved DNA from the said sub-step (i).

5. (canceled)

6. A method of diagnosing colorectal neoplasm, screening for the onset or risk to the onset of colorectal neoplasm or assessing the development or prognosis of colorectal neoplasm in a subject, said method comprises the following steps:
(a). obtaining a biological sample containing DNA from the subject;
(b). treating the DNA in the biological sample obtained from step (a) with a reagent capable of distinguishing between an unmethylated site and a methylated site in the DNA, thereby obtaining a treated DNA;
(c). pre-amplifying at least a portion of at least one target marker within the treated DNA obtained from step (b) with a pre-amplification primer pool, wherein at least a portion of at least one of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(d). if step (c) is present, then quantifying individually methylation level of the at least one target marker based on achieved DNA from step (c); if step (c) is absent, then quantifying individually methylation level of at least one target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
comparing the methylation level of at least one target marker from step (d) respectively with a corresponding reference level, wherein an identical or higher methylation level of one or more of the target marker(s) relative to its corresponding reference level indicates that the subject has colorectal neoplasm, or is at the onset or at a risk to the onset of colorectal neoplasm, or develops or with an increased probability of developing colorectal neoplasm, or has poor prognosis or at a risk to poor prognosis of colorectal neoplasm.

7. A method of monitoring treatment response in a subject who is receiving treatment of colorectal neoplasm, comprising the following steps:
(e). obtaining a biological sample containing DNA from the subject;
(f). treating the DNA in the biological sample obtained from step (a) with a reagent capable of distinguishing between an unmethylated site and a methylated site in the DNA, thereby obtaining a treated DNA;
(g). pre-amplifying at least a portion of at least one target marker within the treated DNA obtained from step (b) with a pre-amplification primer pool, wherein at least a portion of at least one of the target marker(s) is pre-amplified, and the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; wherein step (c) is present or absent;
(h). if step (c) is present, then quantifying individually methylation level of the at least one target marker based on achieved DNA from step (c); if step (c) is absent, then quantifying individually methylation level of at least one target marker within the treated DNA obtained from step (b), wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5; and
comparing the methylation level of at least one target marker from step (d) respectively with a corresponding methylation level of one or more of the target marker(s) obtained from the same subject prior to the treatment which is quantified by repeating step (a), step (b), optionally step (c), and step (d) with respect to a biological sample containing DNA obtained from the subject prior to the treatment, wherein a lower methylation level of one or more of the target marker(s) relative to its corresponding methylation level prior to the treatment indicates that the subject is responsive to the treatment.

8. The method of claim 1, wherein the at least one target marker comprises multiple target markers, wherein the multiple target markers comprise at least two markers selected from the group consisting of Septin9, BCAT1, and IKZF1% preferably wherein the multiple target markers further comprise one or more additional markers selected from the group consisting of BCAN, PKNOX2, VAV3, NDRG4, and IRF4; and/or preferably wherein the multiple target markers further comprise one or more additional markers selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP.

9-11. (canceled)

12. The method claim 1, wherein the respective target marker comprises or is: a) the respective region defined by Hg19 coordinates as set forth below:

| Target Marker | Hg19 Coordinate |
|---|---|
| NDRG4 | chr16: 58496750-58547532 |
| BCAT1 | chr12: 24964295-25102393 |
| IKZF1 | chr7: 50343720-50472799 |
| Septin9 | chr17: 75276651-75496678 |
| SDC2 | chr8: 97505579-97624000 |
| VAV3 | chr1: 108113782-108507766 |
| IRF4 | chr6: 391739-411447 |
| TMEFF2 | chr2: 192813769-193060435 |
| SALL1 | chr16: 51169886-51185278 |
| BCAN | chr1: 156611182-156629324 |
| POU4F2 | chr4: 147560045-147563626 |
| PKNOX2 | chr11: 125034583-125303285 |
| ASCL4 | chr12: 108168162-108170421 |
| KCNA6 | chr12: 4918342-4960277 |
| SOX1 | chr13: 112721913-112726020 |
| HS3ST2 | chr16: 22825498-22927659 |
| FGF12 | chr3: 191857184-192485553 |
| KCTD8 | chr4: 44175926-44450824 |
| HMX1 | chr4: 8847802-8873543 |
| MARCH11 | chr5: 16067248-16180871 |
| CRHBP | chr5: 76248538-76276983 |
| NKX2-6 | chr8: 23559964-23564111 |
| SLC24A2 | chr9: 19507450-19786926 |
| INTERGENIC REGION 1 | chr6: 19679885-19693988 |
| INTERGENIC REGION 2 | chr10: 130082033-130087148 |
| INTERGENIC REGION 3 | chr10: 133107880-133113966 |
| INTERGENIC REGION 4 | chr7: 152620588-152624685 |
| INTERGENIC REGION 5 | chr8: 70945014-70949177, |

and 5 kb upstream of the respective start site and 5 kb downstream of the respective end site of each region described above, or b) a bisulfite converted counterpart of a), or c) a MSRE treated counterpart of a).

13. The method of claim 1, wherein the DNA comprises genomic DNA or cell-free DNA.

14. The method of claim 13, wherein the cell-free DNA comprises circulating tumor DNA.

15. The method of claim 13, wherein the target marker in the cell-free DNA is present in the biological sample in an amount no more than 1 ng, 0.8 ng, 0.6 ng, 0.4 ng, 0.2 ng, 0.1 ng, 0.08 ng or no more than 0.04 ng.

16-17. (canceled)

18. The method of claim 1, wherein the biological sample is selected from the group consisting of a tissue section, biopsy, a paraffin-embedded tissue, a body fluid, colonic effluent, a surgical resection sample, an isolated blood cell, a cell isolated from blood, and any combination thereof;
preferably wherein the body fluid is selected from the group consisting of whole blood, blood serum, blood plasma, urine, mucus, saliva, peritoneal fluid, pleural fluid, chest fluid, synovial fluid, cerebrospinal fluid, thoracentesis fluid, abdominal fluid, and any combination thereof.

19-21. (canceled)

22. The method of claim 1, wherein the reagent capable of distinguishing between an unmethylated site and a methylated site in the DNA selectively modifies at unmethylated cytosine residue(s) at the CpG site(s) to produce modified residue(s) but does not significantly modify methylated cytosine residue(s).

23-27. (canceled)

28. The method of claim 4, wherein the pre-amplification primer pool comprises at least one methylation-specific primer pair;
preferably wherein the at least one methylation-specific primer pair comprises a forward primer and a reverse primer each comprising an oligonucleotide sequence that hybridizes under stringent conditions, moderately stringent conditions, or highly stringent conditions to at least 9 consecutive nucleotides of one of the target marker(s), wherein the at least 9 consecutive nucleotides of one of the target marker(s) comprise at least one CpG site.

29-36. (canceled)

37. The method of claim 6, wherein if step (c) is present, then the quantifying of step (d) comprises amplifying the achieved DNA from step (c) using quantification primer pair(s) and a DNA polymerase, wherein the at least a portion of the achieved DNA is amplified; if step (c) is absent, then the quantifying of step (d) comprises amplifying the at least one target marker within the treated DNA obtained from step (b) using quantification primer pair(s) and a DNA polymerase; or
preferably wherein if step (c) is present, then the quantification primer pair(s) used in step (d) is (are) capable of hybridizing to at least 9 consecutive nucleotides of the achieved DNA from step (c) under stringent conditions, moderately stringent conditions, or highly stringent conditions; if step (c) is absent, then the quantification primer pair(s) used in step (d) is (are) capable of hybridizing to at least 9 consecutive nucleotides of the at least one target marker within the treated DNA obtained from step (b) under stringent conditions, moderately stringent conditions, or highly stringent conditions; or
preferably wherein if step (c) is present, then at least one of the quantification primer pair(s) used in step (d) is (are) identical to at least one of the methylation-specific primer pair(s) in the pre-amplification primer pool of step (c); or
preferably wherein if step (c) is present, then the quantification primer pair(s) used in step (d) is (are) designed to amplify at least a portion within the achieved DNA from step (c); if step (c) is absent, then the quantification primer pair(s) used in step (d) is (are) designed to amplify at least a portion within the at least one target marker within the treated DNA obtained from step (b).

38-44. (canceled)

45. The method of claim 1, wherein step (e) comprises comparing Ct value(s) of the target marker(s) of step (d) with a reference Ct value, wherein an identical or lower Ct value of at least one target marker relative to its corresponding reference Ct value indicates that the subject has colorectal neoplasm, is at the onset or at a risk to the onset of colorectal neoplasm, or develops or with an increased probability of developing colorectal neoplasm, or has poor prognosis or at a risk to poor prognosis of colorectal neoplasm; or a higher Ct value of at least one target marker relative to its corresponding Ct value prior to the treatment indicates that the subject who is receiving the treatment of colorectal neoplasm is responsive to the treatment.

46. (canceled)

47. The method of claim 6, wherein if step (c) is present, then the quantifying of step (d) comprises determining the methylation level based on presence or level of a plurality of CpG dinucleotides, TpG dinucleotides, or CpA dinucleotides in the achieved DNA from step (c); if step (c) is absent, then the quantifying of step (d) comprises determining the methylation level of at least one target marker based on presence or level of a plurality of CpG dinucleotides, TpG dinucleotides, or CpA dinucleotides in the at least one target marker within the treated DNA obtained from step (b); or
preferably wherein if step (c) is present, then the quantifying of step (d) comprises determining methylation level of cytosine residue(s) based on presence or level of one or more CpG dinucleotides in the achieved DNA from step (c): if step (c) is absent, then the quantifying of step (d) comprises determining methylation level of cytosine residue(s) based on presence or level of one or more CpG dinucleotides in the at least one target marker within the treated DNA obtained from step (b); or
preferably wherein if step (c) is present, then the quantifying of step (d) is performed by partitioning the achieved DNA from step (c) into a plurality of fractions; if step (c) is absent, then the quantifying of step (d) is performed by partitioning the at least one target marker within the treated DNA obtained from step (b) into a plurality of fractions.

48-50. (canceled)

51. The method of claim 1, wherein the colorectal neoplasm is a colorectal cancer, a large colorectal adenoma, and/or a sessile serrated polyp

52-53. (canceled)

54. A kit for diagnosing colorectal neoplasm, screening for the onset or risk to the onset of colorectal neoplasm or assessing the development or prognosis of colorectal neoplasm, comprising:
(i). a first reagent for treating a DNA, wherein the first reagent is capable of distinguishing between an unmethylated site and a methylated site in the DNA;
(j). optionally a first primer pool comprising at least one primer pair for pre-amplifying at least one target sequence in at least one target marker selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5, wherein the at least one primer pair is capable of hybridizing under stringent conditions, moderately stringent conditions, or highly stringent conditions to at least 9 consecutive nucleotides of the at least one target sequence treated by the first reagent, and wherein the target sequence comprises at least one CpG site; and
a second reagent, wherein if the first primer pool is present, then the second reagent is for quantifying methylation level of the at least one target marker pre-amplified by the first primer pool; if the first primer pool is absent, then the second reagent is for quantifying methylation level of at least one target marker within the DNA treated by the first reagent, wherein the at least one target marker comprises one or more markers selected from the group consisting of Septin9, BCAT1, IKZF1, BCAN, VAV3, IRF4, POU4F2, SALL1, PKNOX2, SDC2, ASCL4, TMEFF2, SLC24A2, NDRG4, NKX2-6, KCNA6, SOX1, HS3ST2, FGF12, KCTD8, HMX1, MARCH11, CRHBP, INTERGENIC REGION 1, INTERGENIC REGION 2, INTERGENIC REGION 3, INTERGENIC REGION 4, and INTERGENIC REGION 5.

55. (canceled)

56. The kit of claim 54, wherein if the first primer pool is present, then the second reagent comprises a second primer pool comprising multiple quantification primer pairs capable of hybridizing under stringent conditions, moderately stringent conditions, or highly stringent conditions to at least 9 consecutive nucleotides of the at least one target sequence pre-amplified by the first primer pool; if the first primer pool is absent, then the second reagent comprises a third primer pool comprising multiple quantification primer pairs capable of hybridizing under stringent conditions, moderately stringent conditions, or highly stringent conditions to at least 9 consecutive nucleotides of the at least one target sequence of the at least one target marker within the DNA treated by the first reagent.

57. The kit of claim 56, wherein at least one of the quantification primer pairs in the second primer pool is identical to at least one of the primer pairs in the first primer pool; or
preferably wherein if the first primer pool is present, then quantification primer pairs of the second primer pool are designed to amplify at least a portion within the at least one target sequence pre-amplified by the first primer pool; if the first primer pool is absent, then quantification primer pairs of the third primer pool are designed to amplify at least a portion within the at least one target sequence of the at least one target marker within the DNA treated by the first reagent.

58. (canceled)

59. The kit of claim 54, wherein the first, second, or third primer pool comprises at least one methylation-specific primer pair.

60-70. (canceled)

71. The kit of claim 54, wherein the multiple target markers further comprise one or more additional markers selected from the group consisting of BCAN, PKNOX2, VAV3, NDRG4, and IRF4; or
wherein the multiple target markers further comprise one or more additional markers selected from the group consisting of POU4F2, SALL1, SDC2, ASCL4, INTERGENIC REGION 1, TMEFF2, INTERGENIC REGION 4, NKX2-6, INTERGENIC REGION 5, SLC24A2, INTERGENIC REGION 2, INTERGENIC REGION 3, KCNA6, SOX1, HS3ST2, FGF12, KCTD8, HMX1, MARCH11, and CRHBP.

72. (canceled)

73. The kit of claim 54, wherein the respective target marker comprises or is: a) the respective region defined by Hg19 coordinates as set forth below:

| Target Marker | Hg19 Coordinate |
|---|---|
| NDRG4 | chr16: 58496750-58547532 |
| BCAT1 | chr12: 24964295-25102393 |
| IKZF1 | chr7: 50343720-50472799 |
| Septin9 | chr17: 75276651-75496678 |
| SDC2 | chr8: 97505579-97624000 |
| VAV3 | chr1: 108113782-108507766 |
| IRF4 | chr6: 391739-411447 |
| TMEFF2 | chr2: 192813769-193060435 |
| SALL1 | chr16: 51169886-51185278 |
| BCAN | chr1: 156611182-156629324 |
| POU4F2 | chr4: 147560045-147563626 |
| PKNOX2 | chr11: 125034583-125303285 |
| ASCL4 | chr12: 108168162-108170421 |
| KCNA6 | chr12: 4918342-4960277 |
| SOX1 | chr13: 112721913-112726020 |
| HS3ST2 | chr16: 22825498-22927659 |
| FGF12 | chr3: 191857184-192485553 |
| KCTD8 | chr4: 44175926-44450824 |
| HMX1 | chr4: 8847802-8873543 |
| MARCH11 | chr5: 16067248-16180871 |
| CRHBP | chr5: 76248538-76276983 |
| NKX2-6 | chr8: 23559964-23564111 |
| SLC24A2 | chr9: 19507450-19786926 |
| INTERGENIC REGION 1 | chr6: 19679885-19693988 |
| INTERGENIC REGION 2 | chr10: 130082033-130087148 |
| INTERGENIC REGION 3 | chr10: 133107880-133113966 |
| INTERGENIC REGION 4 | chr7: 152620588-152624685 |
| INTERGENIC REGION 5 | chr8: 70945014-70949177, |

and 5 kb upstream of the respective start site and 5 kb downstream of the respective end site of each region described above, or b) a bisulfite converted counterpart of a), or c) a MSRE treated counterpart of a).

74-78. (canceled)
